(19) United States
(12) Patent Application Publication
(10) Pub. No.: US 2025/0331415 A1
(43) Pub. Date: Oct. 23, 2025

(54) ORGANIC COMPOUND, AN ELECTRONIC ELEMENT USING SAME, AND ELECTRONIC APPARATUS

(71) Applicant: SHAANXI LIGHTE OPTOELECTRONICS MATERIAL CO., LTD., Xi'an, Shaanxi (CN)

(72) Inventors: Xianbin XU, Xi'an, Shaanxi (CN); Lei YANG, Xi'an, Shaanxi (CN)

(21) Appl. No.: 18/871,232

(22) PCT Filed: Sep. 27, 2023
(86) PCT No.: PCT/CN2023/122150

§ 371 Date: Dec. 3, 2024

(30) Foreign Application Priority Data
Apr. 11, 2023 (CN) 202310384311.1

(51) Int. Cl. H10K 85/60; C07D 307/91; H10K 50/11; H10K 50/15

(52) U.S. Cl. CPC H10K 85/636; C07D 307/91; H10K 85/633; H10K 50/11; H10K 50/15; H10K 85/615; H10K 85/626; H10K 85/6574

(57) ABSTRACT

Provided are an organic compound, an electronic element using same, and an electronic apparatus. The organic compound has a structure shown in a formula I. The organic compound is applied to an organic electroluminescent device, so that the performance of the device can be significantly improved.

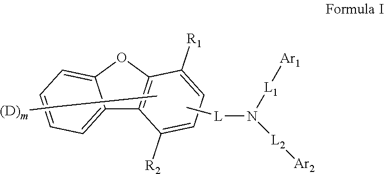

### CROSS-REFERENCE TO RELATED APPLICATIONS

The present disclosure claims the priority of Chinese patent application No. 2023103843111 filed on Apr. 11, 2023, which is incorporated herein by reference in its entirety as a part of this disclosure.

### FIELD OF THE INVENTION

The present disclosure relates to the technical field of organic electroluminescence, and specifically to an organic compound and an electronic element and an electronic apparatus using the same.

### BACKGROUND OF THE INVENTION

With the development of electronic technology and the progress of material science, the application range of electronic elements and devices used to realize electroluminescence or photoelectric conversion is more and more extensive. This type of electronic element and device usually comprises a cathode and an anode disposed opposite to each other, and a functional layer disposed between the cathode and the anode. The functional layer is composed of multiple organic or inorganic film layers, and generally includes an energy conversion layer, a hole transport layer located between the energy conversion layer and the anode, and an electron transport layer located between the energy conversion layer and the cathode.

Taking an organic electroluminescent device as an example, it generally includes an anode, a hole transport layer, an organic light-emitting layer, an electron transport layer, and a cathode stacked in sequence. When a voltage is applied to the anode and cathode, an electric field is generated between the two electrodes. Under the influence of the electric field, electrons on the cathode side move towards the electroluminescent layer, and holes on the anode side also move towards the luminescent layer. Electrons and holes combine in the electroluminescent layer to form excitons, which are in an excited state and release energy outward, thereby causing the electroluminescent layer to emit light externally.

In the prior art, materials that can be used in an organic electroluminescent device are disclosed in WO2016087017A1, KR1020110110508A, CN111094234A, etc. However, it is still necessary to continue developing new materials to further improve the performance of electronic elements.

### SUMMARY OF THE INVENTION

The objective of the present disclosure is to provide an organic compound and an electronic element and an electronic apparatus using the organic compound. The application of the organic compound in organic electroluminescent devices can improve the performance of the organic electroluminescent devices.

A first aspect of the present disclosure provides an organic compound having a structure shown in a Formula I:

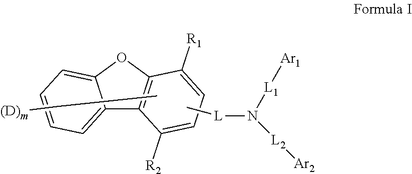

wherein R₁ and R₂ are the same or different, and are each independently a substituted or unsubstituted aryl having 6 to 12 carbon atoms;
the substituent(s) of R₁ and R₂ are the same or different, and are each independently selected from a deuterium, a halogen group, a cyano, or an alkyl having 1 to 5 carbon atoms;
L, L₁, and L₂ are the same or different, and are each independently selected from a single bond, a substituted or unsubstituted arylene having 6 to 30 carbon atoms, or a substituted or unsubstituted heteroarylene having 3 to 30 carbon atoms;
Ar₁ and Ar₂ are the same or different, and are each independently selected from a substituted or unsubstituted aryl having 6 to 30 carbon atoms, or a substituted or unsubstituted heteroaryl having 3 to 30 carbon atoms;
the substituent(s) of L, L₁, L₂, Ar₁ and Ar₂ are the same or different, and are each independently selected from a deuterium, a halogen group, a cyano, an alkyl having 1 to 10 carbon atoms, a deuteroalkyl having 1 to 10 carbon atoms, a trialkylsilyl having 3 to 12 carbon atoms, a haloalkyl having 1 to 10 carbon atoms, a cycloalkyl having 3 to 10 carbon atoms, an aryl having 6 to 20 carbon atoms, or a heteroaryl having 3 to 20 carbon atoms;
m represents the number of D, and m is selected from 0, 1, 2, 3, 4, or 5.

A second aspect of the present disclosure provides an electronic element, comprising an anode and a cathode disposed opposite to each other, and a functional layer disposed between the anode and the cathode; the functional layer contains the organic compound described in the first aspect of the present disclosure.

A third aspect of the present disclosure provides an electronic apparatus, comprising the electronic element described in the second aspect of the present disclosure.

The main structural feature of the compound of the present disclosure is the triple substitution on the same benzene ring in a dibenzofuranyl moiety, wherein the substituents at positions 1 and 4 of the dibenzofuranyl are selected from a simple aryl group, and the dibenzofuranyl is connected to an aromatic amine group at its position 2 or 3. On one hand, the substituents at positions 1 and 4 of the dibenzofuranyl can enhance the intermolecular interaction force, thus increasing the carrier mobility of the compound; on the other hand, the dibenzofuranyl is connected to an aromatic amine at its position 2 or 3, and to a simple aryl at its positions 1 and 4, and such a structure results in less distortion of the molecule compared to the structure of the aromatic amine in which the carbon atoms of the two sides are substituted by the aryl group at the same time, and therefore, the molecules stack more tightly, which endows the compound with a higher carrier mobility. Application of the compound of the present disclosure as a hole transport host material or a second hole transport material in a mixed host material can improve the carrier balance in a light-emitting layer, broaden the carrier recombination region, improve the exciton generation and utilization efficiency, and improve the light-emitting efficiency and lifetime of a device.

The other features and advantages of the present invention will be described in detail in the following detailed description of the embodiments.

### BRIEF DESCRIPTION OF THE DRAWINGS

The drawings are provided for further understanding of the present disclosure and constitute a part of the specification, and together with the following detailed description, are used to explain the present disclosure, but do not constitute a limitation of the present disclosure.

FIG. 1 is a schematic structural diagram of an organic electroluminescent device according to an embodiment of the present disclosure.

FIG. 2 is a schematic diagram of a first electronic apparatus according to an embodiment of the present disclosure.

FIG. 3 is a schematic structural diagram of a photoelectric conversion device according to an embodiment of the present disclosure.

FIG. 4 is a schematic diagram of a second electronic apparatus according to an embodiment of the present disclosure.

### NOTE OF REFERENCE SIGNS

100: Anode; 200: Cathode; 300: Functional layer; 310: Hole injection layer; 320: Hole transport layer; 321: First hole transport layer; 322: Second hole transport layer; 330: Organic light-emitting layer; 340: Electron transport layer; 350: Electron injection layer; 360: Photoelectric conversion layer; 400: First electronic apparatus; 500: Second electronic apparatus

### DETAILED DESCRIPTION OF THE EMBODIMENTS

Exemplary embodiments will now be described more comprehensively with reference to the accompanying drawings. The exemplary embodiments, however, can be implemented in a variety of forms and should not be interpreted as being limited to the examples set forth herein. On the contrary, these examples are provided to make the present disclosure more comprehensive and complete, and to convey the concepts of these exemplary embodiments fully to those of ordinary skill in the art. Features, structures, or characteristics described herein can be combined in one or more embodiments in any suitable manner. In the following description, various specific details are provided to give a full understanding of the embodiments of the present disclosure.

In a first aspect, the present disclosure provides an organic compound having a structure shown in a Formula I:

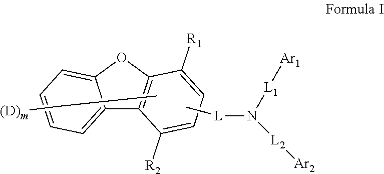

wherein R₁ and R₂ are the same or different, and are each independently a substituted or unsubstituted aryl having 6 to 12 carbon atoms;
the substituent(s) of R₁ and R₂ are the same or different, and are each independently selected from a deuterium, a halogen group, a cyano, or an alkyl having 1 to 5 carbon atoms;
L, L₁, and L₂ are the same or different, and are each independently a single bond, a substituted or unsubstituted arylene having 6 to 30 carbon atoms, or a substituted or unsubstituted heteroarylene having 3 to 30 carbon atoms;
Ar₁ and Ar₂ are the same or different, and are each independently selected from a substituted or unsubstituted aryl having 6 to 30 carbon atoms, or a substituted or unsubstituted heteroaryl having 3 to 30 carbon atoms;
the substituent(s) of L, L₁, L₂, Ar₁ and Ar₂ are the same or different, and are each independently selected from a deuterium, a halogen group, a cyano, an alkyl having 1 to 10 carbon atoms, a deuteroalkyl having 1 to 10 carbon atoms, a trialkylsilyl having 3 to 12 carbon atoms, a haloalkyl having 1 to 10 carbon atoms, a cycloalkyl having 3 to 10 carbon atoms, an aryl having 6 to 20 carbon atoms, or a heteroaryl having 3 to 20 carbon atoms;
m represents the number of D, and m is selected from 0, 1, 2, 3, 4, or 5.

In the present disclosure, D represents a deuterium.

In the present disclosure, the descriptive expression “be . . . each independently” may be used interchangeably with the descriptive expressions “be . . . respectively and independently”, and all these expressions should be interpreted in a broad sense. They can not only mean that, in different groups, specific options expressed by the same symbols are mutul non-influential, but also mean that in the same group, specific options expressed by the same symbols are mutul non-influential. For example,

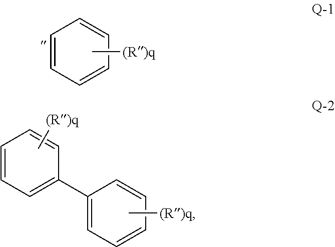

in which each q is independently 0, 1, 2, or 3, and each R″ is independently selected from a hydrogen, a deuterium, a fluorine, and a chlorine”, means that the Formula Q-1 represents that there are q substituents R″ on a benzene ring, and each R″ can be the same or different, with mutual non-influence between the options for each R″; Formula Q-2 represents that there are q substituents R″ on each benzene ring of biphenyl, and the number q of substituents R″ on the two benzene rings can be the same or different and each R″ can be the same or different, with mutual non-influence between the options for each R″.

In the present disclosure, such a term “substituted or unsubstituted” means that the functional group defined by the term may or may not have a substituent (hereinafter referred to as Rc for ease of description). For example, “a substituted or unsubstituted aryl” means an aryl having a substituent Rc or an unsubstituted aryl. Among them, the above substituent, i.e., Rc, may be, for example, a deuterium, a halogen group, a cyano, an alkyl, a trialkylsilyl, a haloalkyl, a cycloalkyl, an aryl, a heteroaryl, and the like.

In the present disclosure, the number of carbon atoms in a substituted or unsubstituted functional group refers to the number of all carbon atoms. For example, if L₁ is a substituted arylene having 12 carbon atoms, the number of all carbon atoms of the arylene and the substituents thereon is 12.

In the present disclosure, an aryl refers to any functional group or substituent derived from an aromatic carbon ring. An aryl may be a monocyclic aryl (e.g., phenyl) or a polycyclic aryl. In other words, an aryl may be a monocyclic aryl, a fused cycloaryl, two or more monocyclic aryls linked by carbon-carbon bond, a monocyclic aryl and a fused cycloaryl linked by carbon-carbon bond, or two or more fused cycloaryls linked by carbon-carbon bond. That is, unless otherwise specified, two or more aromatic groups linked by carbon-carbon bond may also be regarded as an aryl in the present disclosure. Among them, a fused cycloaryl may include, for example, a bicyclic fused aryl (e.g., naphthyl), a tricyclic fused aryl (e.g., phenanthryl, fluorenyl, anthryl), etc. For example, in the present disclosure, biphenyl, terphenyl and the like belong to an aryl. Examples of an aryl include, but are not limited to, a phenyl, a naphthyl, a fluorenyl, anthracenyl, a phenanthryl, a biphenyl, a terphenyl, a benzo[9,10]phenanthryl, a pyrenyl, a benzofluoranthryl, chrysenyl, spirobifluorenyl, etc. In the present disclosure, “an arylene” involved refers to a divalent group formed by further removing one hydrogen atom from an aryl.

In the present disclosure, a substituted aryl may mean that one or more than two hydrogen atoms in an aryl group are replaced by a group such as a deuterium atom, a halogen group, a cyano, an aryl, a heteroaryl, a trialkylsilyl, an alkyl, a cycloalkyl, a haloalkyl, a deuteroalkyl, etc. Specific examples of an aryl substituted with a heteroaryl include, but are not limited to a phenyl substituted with a dibenzofuranyl, a phenyl substituted with a dibenzothienyl, a phenyl substituted with a pyridyl, etc. It should be understood that the number of carbon atoms in a substituted aryl refers to the total number of all carbon atoms of an aryl and the substituents on the aryl. For example, a substituted aryl having 18 carbon atoms, refers to the number of all carbon atoms of the aryl and the substituents thereof is 18.

In the present disclosure, “a heteroaryl” refers to a monovalent aromatic ring containing at least one heteroatom or a derivative thereof. The heteroatom may be one or more of B, O, N, P, Si, Se, and S. A heteroaryl may be a monocyclic heteroaryl or a polycyclic heteroaryl. In other words, a heteroaryl may be a single aromatic ring system, or multiple aromatic ring systems linked by carbon-carbon bond, with any of the aromatic ring systems being an aromatic monocyclic ring or an aromatic fused ring. For example, a heteroaryl may include, a thienyl, a furyl, a pyrrolyl, an imidazolyl, a thiazolyl, an oxazolyl, an oxadiazolyl, a triazolyl, a pyridyl, a dipyridyl, a pyrimidinyl, a triazinyl, an acridinyl, a pyridazinyl, a pyrazinyl, a quinolyl, a quinazolinyl, a quinoxalinyl, a phenoxazinyl, a phthalazinyl, a pyridopyrimidinyl, a pyridopyrazinyl, a pyrazinopyrazinyl, an isoquinolyl, an indolyl, a carbazolyl, a benzoxazolyl, a benzimidazolyl, a benzothiazolyl, a benzocarbazolyl, a benzothienyl, a dibenzothienyl, a thienothienyl, a benzofuranyl, a phenanthrolinyl, an isoxazolyl, a thiadiazolyl, a phenothiazinyl, a silafluorenyl, a dibenzofuranyl, a N-phenylcarbazolyl, a N-pyridylcarbazolyl, a N-methylcarbazolyl, etc, but not limited to thereto. In the present disclosure, “a heteroarylene” involved refers to a divalent group formed by further removing one hydrogen atom from a heteroaryl.

In the present disclosure, a substituted aryl may mean that one or more than two hydrogen atoms in the aryl are replaced by a group such as a deuterium, a halogen group, a cyano, an aryl, a heteroaryl, a trialkylsilyl, an alkyl, a cycloalkyl, a haloalkyl, a deuteroalkyl, etc. Specific examples of a heteroaryl substituted with an aryl include, but are not limited to a dibenzofuranyl substituted with a phenyl, a dibenzothienyl substituted with a phenyl, a pyridyl substituted with a phenyl, etc. It should be understood that the number of carbon atoms in a substituted heteroaryl is the total number of all carbon atoms of the heteroaryl group and substituents on the heteroaryl group.

In the present disclosure, the number of the carbon atoms of an aryl as a substituent may be 6 to 20. For example, the number of carbon atoms may be 6, 7, 8, 9, 10, 11, 12, 13, 14, 15, 16, 17, 18, 19, or 20. The specific examples of an aryl as as substituent include, but are not limited to a phenyl, a biphenyl, a naphthyl, an anthracenyl, and a chrysenyl.

In the present disclosure, the number of the carbon atoms of a heteroaryl as a substituent may be 3 to 20. For example, the number of carbon atoms may be 3, 4, 5, 6, 7, 8, 9, 10, 11, 12, 13, 14, 15, 16, 17, 18, 19, or 20. The specific examples of a heteroaryl as as substituent include, but are not limited to a pyridyl, a pyrimidinyl, a carbazolyl, a dibenzofuranyl, a dibenzothienyl, a quinolyl, a quinazolinyl, a quinoxalinyl, and an isoquinolyl.

In the present disclosure, the number of carbon atoms in an alkyl having 1 to 10 carbon atoms can be, for example, 1, 2, 3, 4, 5, 6, 7, 8, 9, or 10. Specific examples of an alkyl include, but are not limited to a methyl, an ethyl, a n-propyl, an isopropyl, a n-butyl, an isobutyl, a tert-butyl, a n-pentyl, an isopentyl, a neopentyl, a n-hexyl, a n-heptyl, a n-octyl, a 2-ethylhexyl, a nonyl, a decyl, a 3,7-dimethyloctyl, etc.

In the present disclosure, a halogen group may be a fluorine, a chlorine, a bromine, or an iodine.

In the present disclosure, specific examples of a trialkylsilyl include, but are not limited to, a trimethylsilyl, a triethylsilyl, etc.

In the present disclosure, specific examples of a haloalkyl group include, but are not limited to, a trifluoromethyl.

In the present disclosure, specific examples of a deuteroalkyl include, but are not limited to, a trideuteromethyl.

In the present disclosure, the number of a cycloalkyl group having 3 to 10 carbon atoms may be, for example, 3, 4, 5, 6, 7, 8, or 10. Specific examples of a cycloalkyl include, but are not limited to, a cyclopentane, a cyclohexane, and an adamantane.

In the present disclosure, a non-positioned bond refers to a single bond “ [symbol] ” extending from the ring system, which represents that one end of the connection bond can connect to any position in the ring system through which the bond passes, and the other end connects to the rest of the compound molecule. For example, as shown in Formula (f) below, the naphthyl represented by Formula (f) is connected to other positions of the molecule through two non-positioned bonds passing through the two rings, which indicates any of possible connection forms shown in Formulae (f-1) to (f-10):

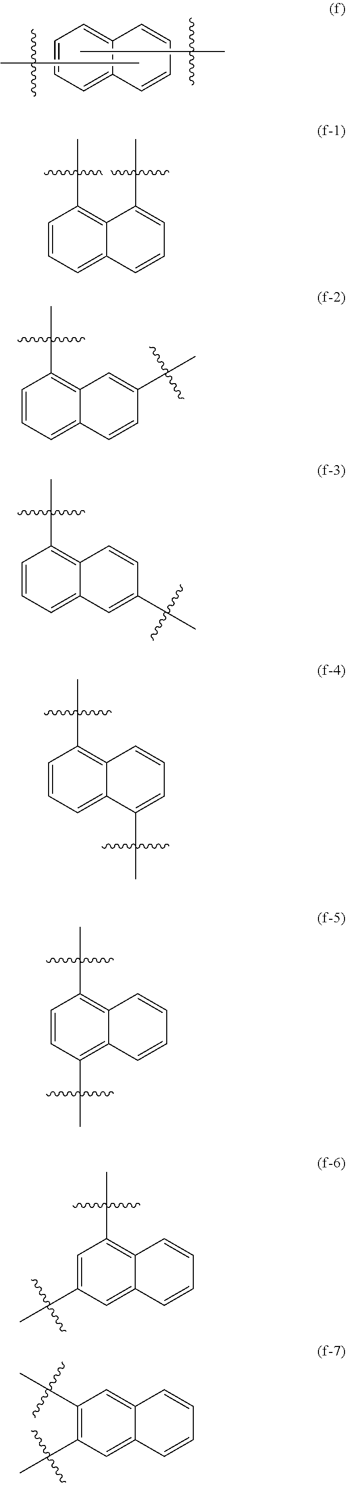

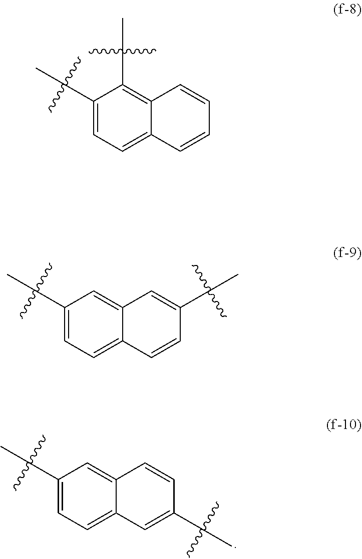 [SMILES: *c1ccc2ccccc2c1* ; *c1ccc2ccc(*)cc2c1 ; *c1ccc2cc(*)ccc2c1]

As another example, as shown in Formula (X′) below, the dibenzofuranyl group represented by Formula (X′) is connected to other positions of the molecule via a non-positioned connecting bond extending from the middle of a side benzene ring, which indicates any of possible connection forms shown in Formulae (X′-1) to (X′-4):

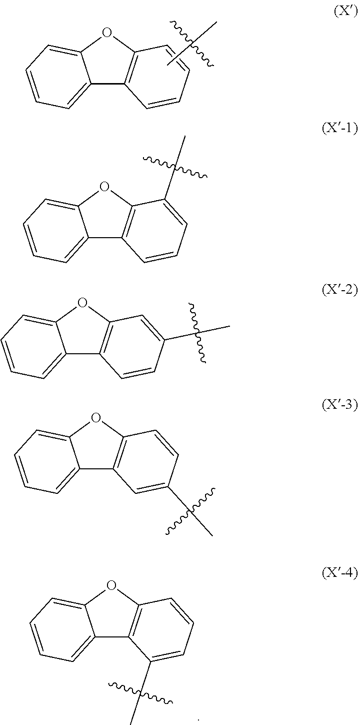

In some embodiments of the present disclosure, the organic compound is selected from the structures shown in a Formula I-I or a Formula I-II:

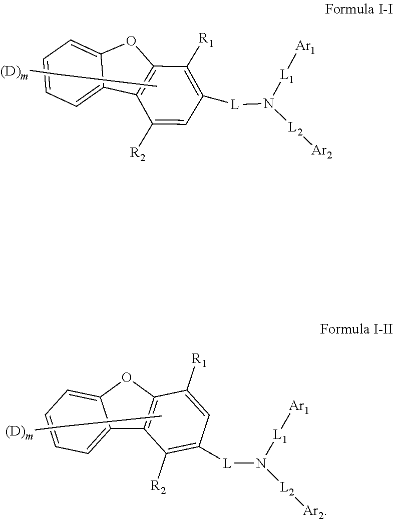

In some embodiments of the present disclosure, R₁ and R₂ are each independently a substituted or unsubstituted phenyl, a substituted or unsubstituted naphthyl, or an unsubstituted biphenyl or a deuterium-substituted biphenyl.

Optionally, substituent(s) of R₁ and R₂ are each independently selected from a deuterium, a fluorine, a cyano, a methyl, an ethyl, an isopropyl, or a tert-butyl.

Optionally, R₁ and R₂ are each independently selected from the group consisting of the following groups:

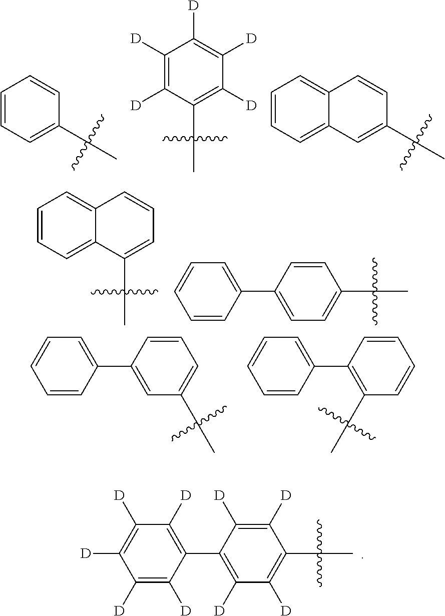

In some embodiments of the present disclosure, L, L₁ and L₂ are each independently selected from a single bond, or a substituted or unsubstituted arylene having 6 to 18 carbon atoms. For example, L, L₁ and L₂ are each independently selected from a single bond, or a substituted or unsubstituted arylene having 6, 7, 8, 9, 10, 11, 12, 13, 14, 15, 16, 17, or 18 carbon atoms.

Optionally, the substituent(s) of L, L₁ and L₂ are each independently selected from a deuterium, a fluorine, a cyano, a trifluoromethyl, a trimethylsilyl, an alkyl having 1 to 5 carbon atoms, or a phenyl.

In some embodiments of the present disclosure, L, L₁ and L₂ are each independently selected from a single bond, a substituted or unsubstituted phenylene, a substituted or unsubstituted naphthylene, a substituted or unsubstituted biphenylene, or a substituted or unsubstituted fluorenylene.

Optionally, the substituent(s) of L, L₁ and L₂ are each independently selected from a deuterium, a fluorine, a cyano, a trifluoromethyl, a trimethylsilyl, a methyl, an ethyl, an isopropyl, a tert-butyl, or a phenyl.

Optionally, L is selected from a single bond, or the group consisting of the following groups:

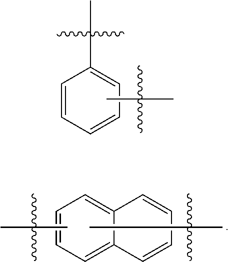

Optionally, L is selected from a single bond, or the group consisting of the following groups:

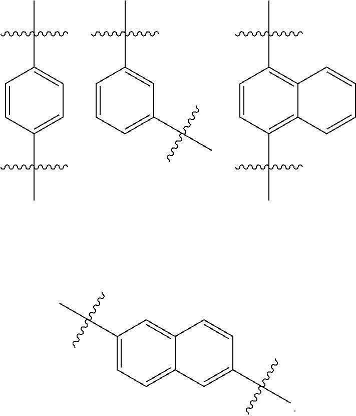

Optionally, L₁ and L₂ are each independently selected from a single bond, or the group consisting of the following groups:

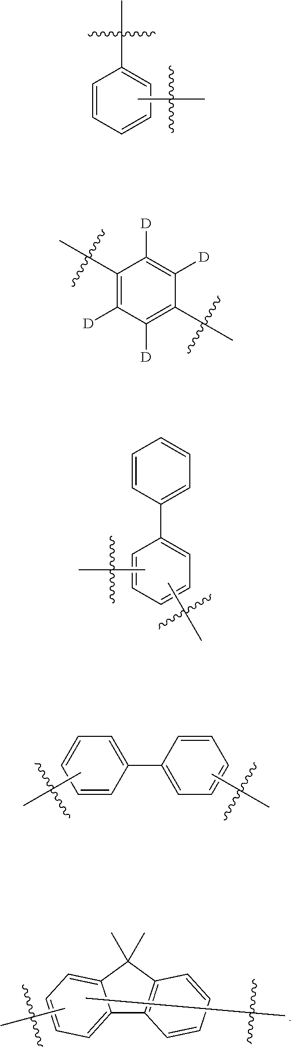

Optionally, L₁ and L₂ are each independently selected from a single bond, or the group consisting of the following groups:

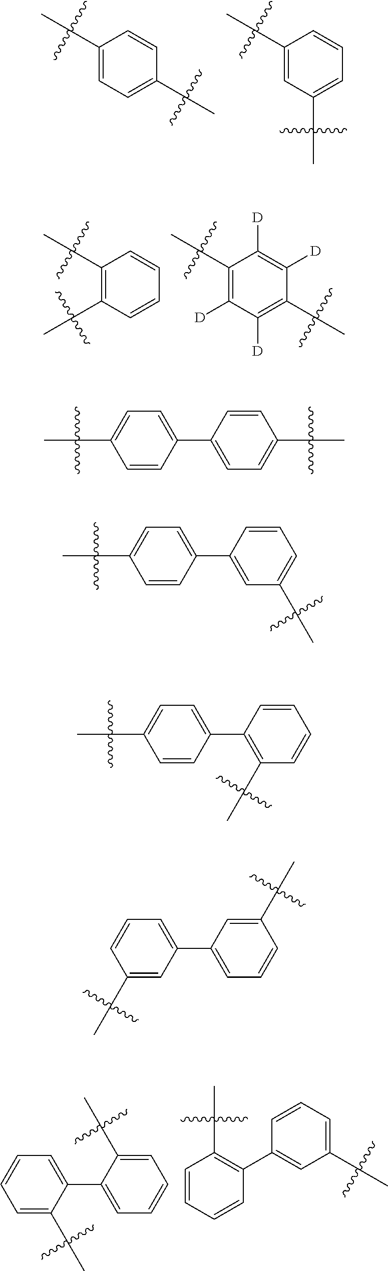

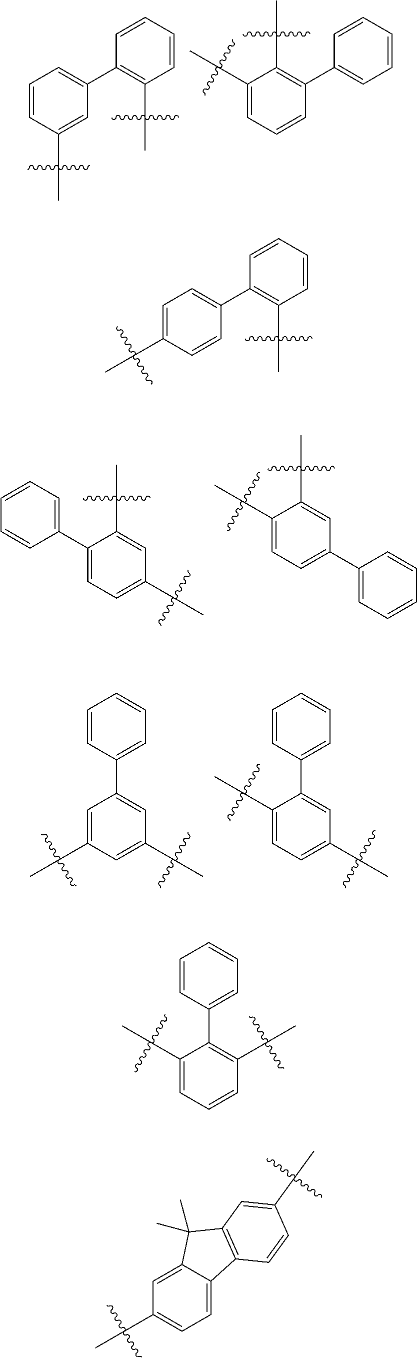

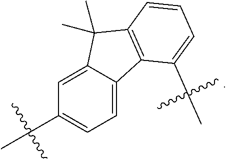 [SMILES: *c1ccc2c(c1)C(C)(C)c1cccc(*)c1-2]

In some embodiments of the present disclosure, Ar₁ and Ar₂ are each independently selected from a substituted or unsubstituted aryl having 6 to 21 carbon atoms. For example, Ar₁ and Ar₂ are each independently selected from a substituted or unsubstituted aryl having 6, 7, 8, 9, 10, 11, 12, 13, 14, 15, 16, 17, 18, 19, 20, or 21 carbon atoms.

Optionally, the substituent(s) of Ar₁ and Ar₂ are each independently selected from a deuterium, a fluorine, a cyano, a trimethylsilyl, an alkyl having 1 to 5 carbon atoms, a haloalkyl having 1 to 5 carbon atoms, a deuteroalkyl having 1 to 5 carbon atoms, or an aryl having 6 to 12 carbon atoms.

Optionally, Ar₁ and Ar₂ are each independently selected from a substituted or unsubstituted phenyl, a substituted or unsubstituted naphthyl, a substituted or unsubstituted biphenyl, a substituted or unsubstituted terphenyl, or a substituted or unsubstituted fluorenyl.

Optionally, the substituent(s) of Ar₁ and Ar₂ are each independently selected from a deuterium, a fluoro, a cyano, a trimethylsilyl, a trifluoromethyl, a methyl, an ethyl, an isopropyl, a tert-butyl, a trideuteromethyl, or a phenyl.

Optionally, Ar₁ and Ar₂ are each independently selected from the group consisting of the following groups:

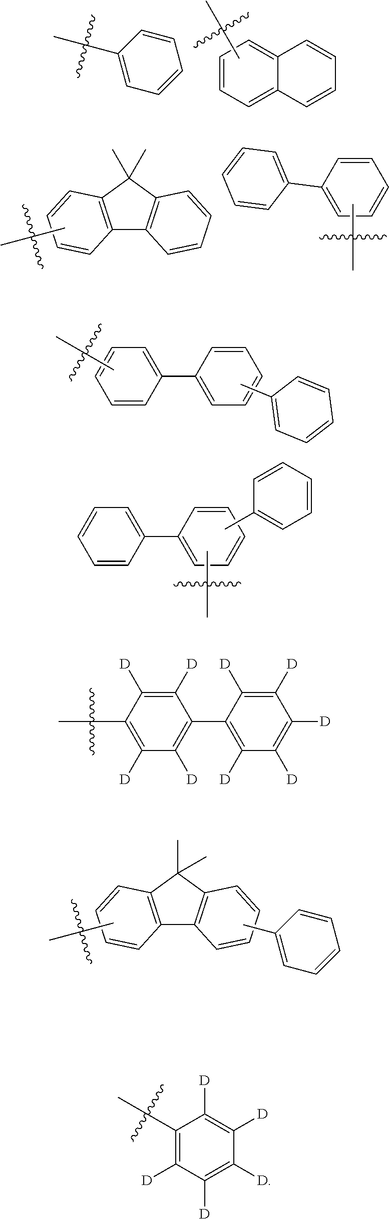

Further optionally, Ar₁ and Ar₂ are each independently selected from the group consisting of the following groups:

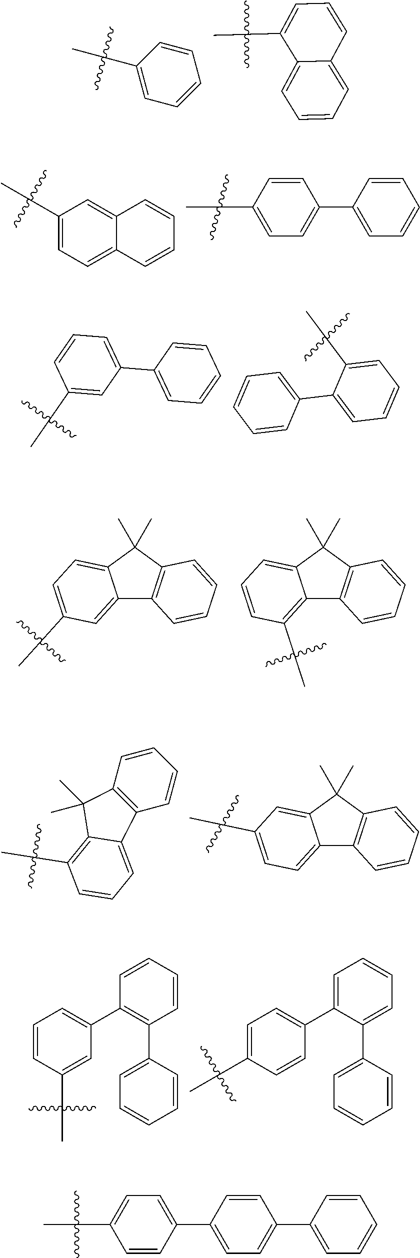

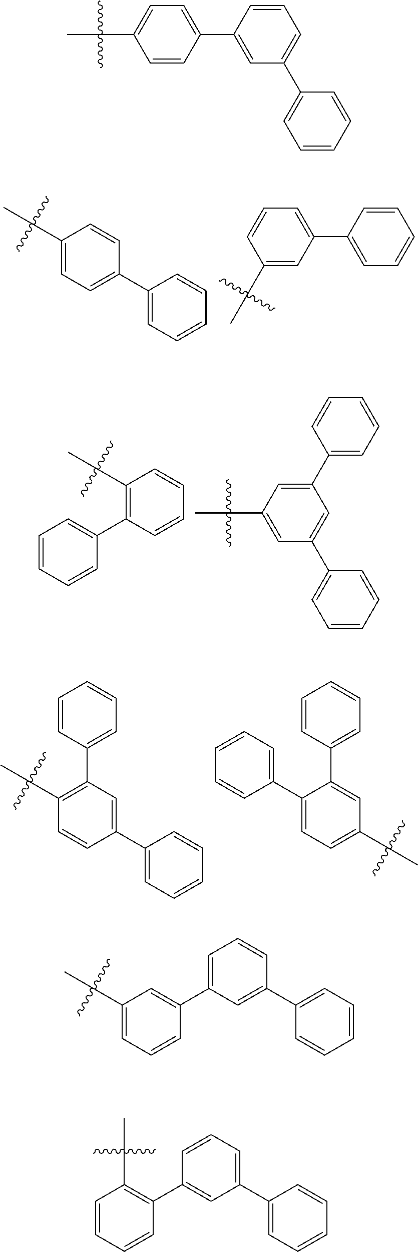

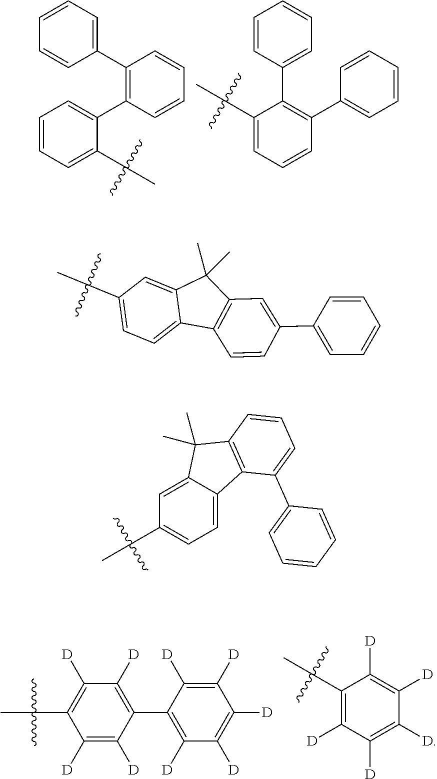

Optionally,

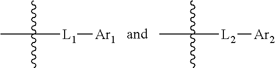

are each independently selected from the group consisting of the following groups:

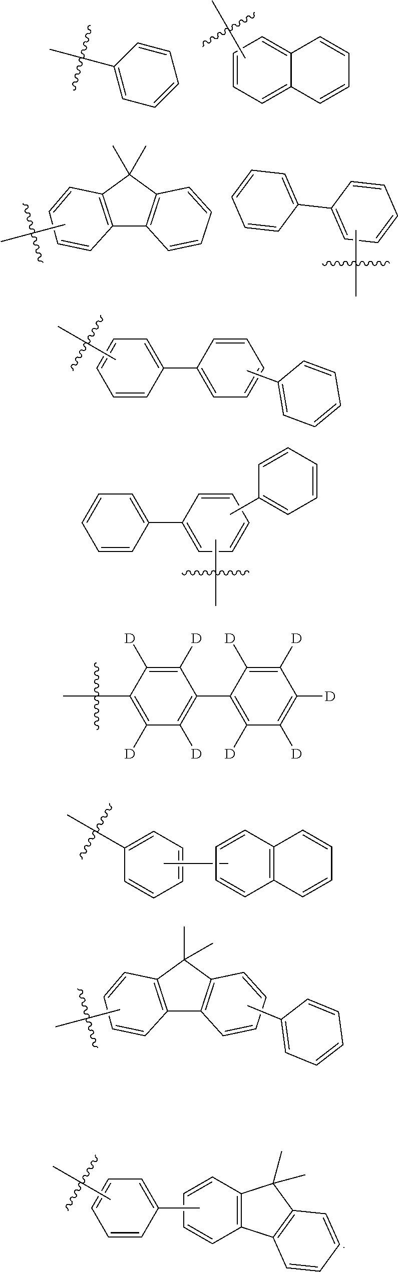

Optionally,

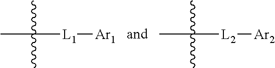

are each independently selected from the group consisting of the following groups:

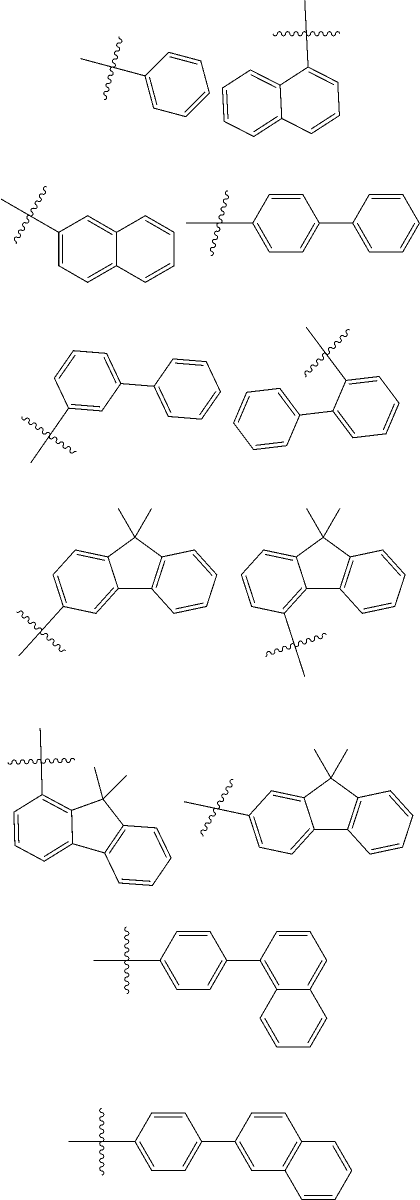

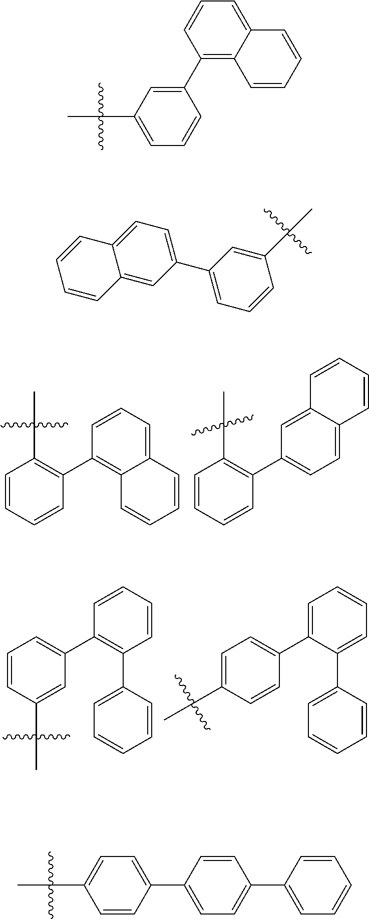

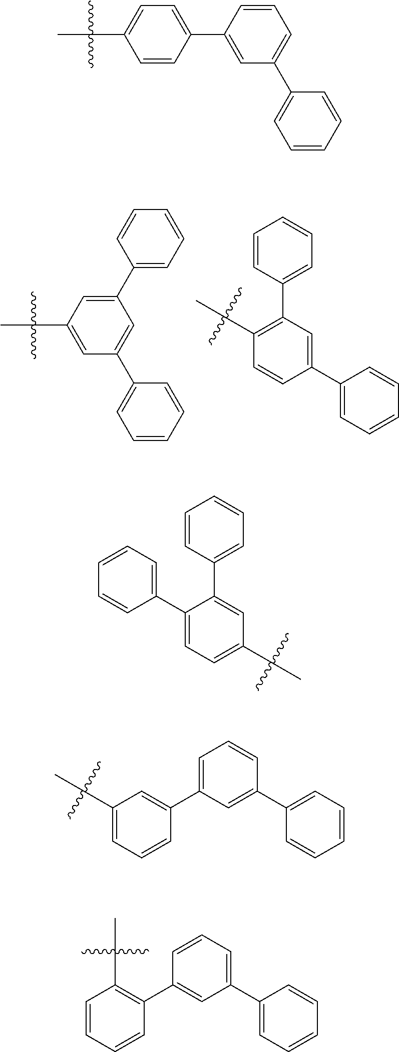 [SMILES: *c1ccc(-c2cccc(-c3ccccc3)c2)cc1 ; *c1cc(-c2ccccc2)cc(-c2ccccc2)c1 ; *c1ccc(-c2ccccc2)cc1-c1ccccc1 ; *c1ccc(-c2ccccc2)c(-c2ccccc2)c1 ; *c1cccc(-c2cccc(-c3ccccc3)c2)c1 ; *c1ccccc1-c1cccc(-c2ccccc2)c1]

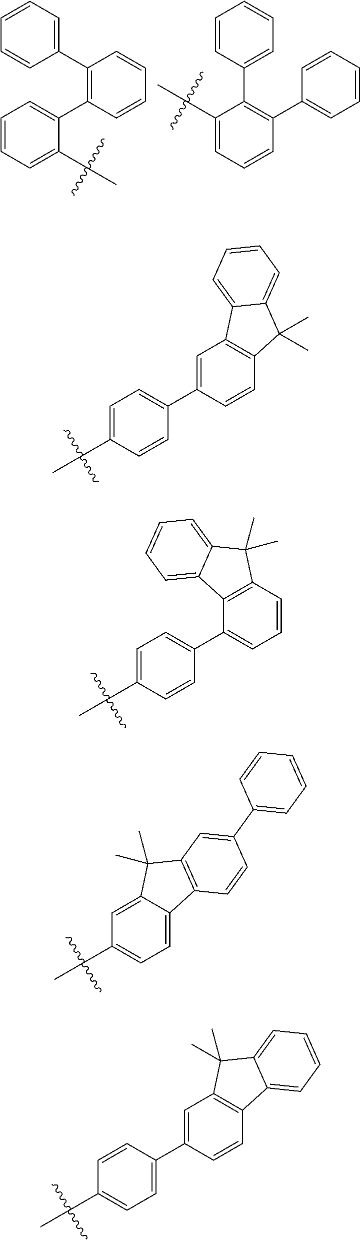

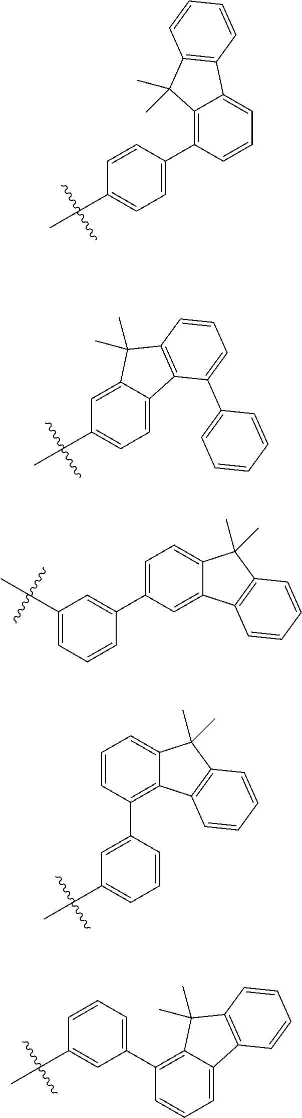 [SMILES: *c1ccc(-c2cccc3c2C(C)(C)c2ccccc2-3)cc1 ; *c1ccc2c(c1)C(C)(C)c1cccc(-c3ccccc3)c1-2 ; *c1cccc(-c2ccc3c(c2)-c2ccccc2C3(C)C)c1 ; *c1cccc(-c2cccc3c2-c2ccccc2C3(C)C)c1 ; *c1cccc(-c2cccc3c2C(C)(C)c2ccccc2-3)c1]

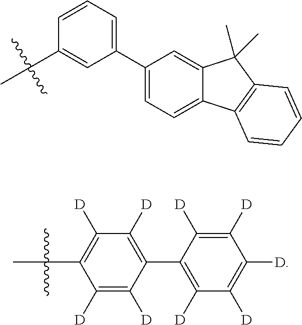

Specifically, the organic compound is selected from the group consisting of the following compounds:

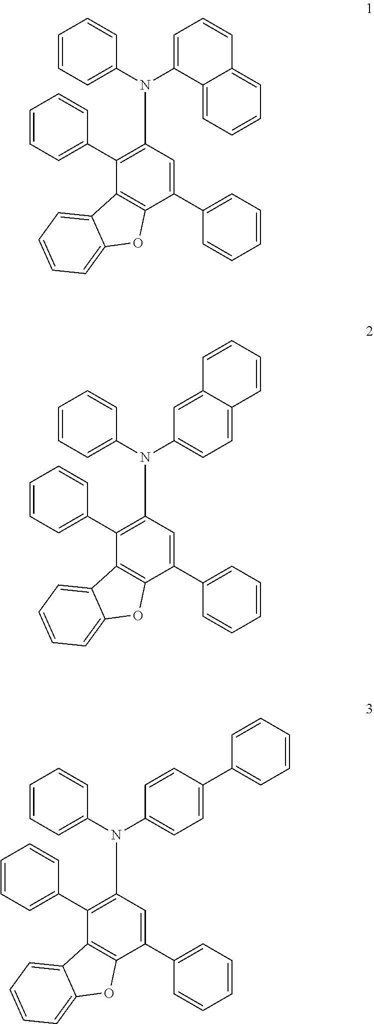 [SMILES: c1ccc(-c2cc(N(c3ccccc3)c3cccc4ccccc34)c(-c3ccccc3)c3c2oc2ccccc23)cc1 ; c1ccc(-c2cc(N(c3ccccc3)c3ccc4ccccc4c3)c(-c3ccccc3)c3c2oc2ccccc23)cc1 ; c1ccc(-c2ccc(N(c3ccccc3)c3cc(-c4ccccc4)c4oc5ccccc5c4c3-c3ccccc3)cc2)cc1]

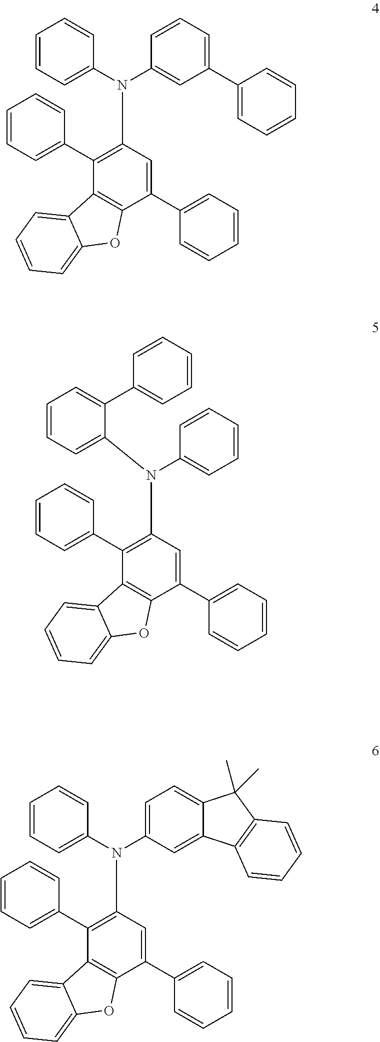 [SMILES: c1ccc(-c2cccc(N(c3ccccc3)c3cc(-c4ccccc4)c4oc5ccccc5c4c3-c3ccccc3)c2)cc1 ; c1ccc(-c2ccccc2N(c2ccccc2)c2cc(-c3ccccc3)c3oc4ccccc4c3c2-c2ccccc2)cc1 ; CC1(C)c2ccccc2-c2cc(N(c3ccccc3)c3cc(-c4ccccc4)c4oc5ccccc5c4c3-c3ccccc3)ccc21]

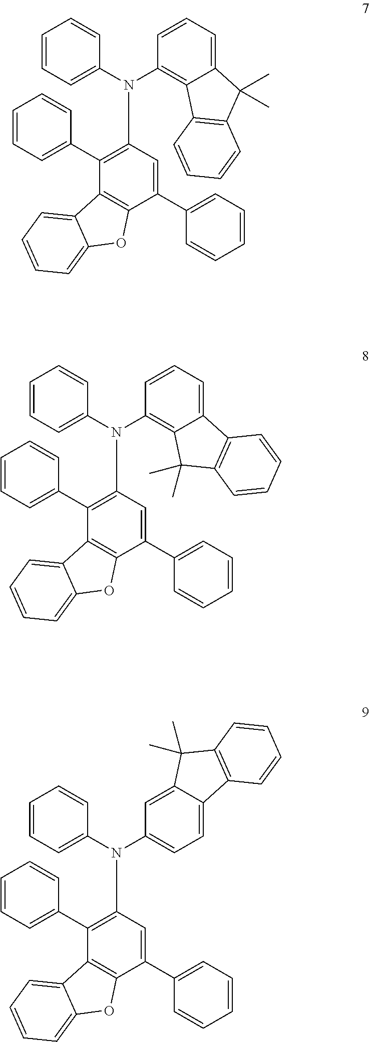 [SMILES: CC1(C)c2ccccc2-c2c(N(c3ccccc3)c3cc(-c4ccccc4)c4oc5ccccc5c4c3-c3ccccc3)cccc21 ; CC1(C)c2ccccc2-c2cccc(N(c3ccccc3)c3cc(-c4ccccc4)c4oc5ccccc5c4c3-c3ccccc3)c21 ; CC1(C)c2ccccc2-c2ccc(N(c3ccccc3)c3cc(-c4ccccc4)c4oc5ccccc5c4c3-c3ccccc3)cc21]

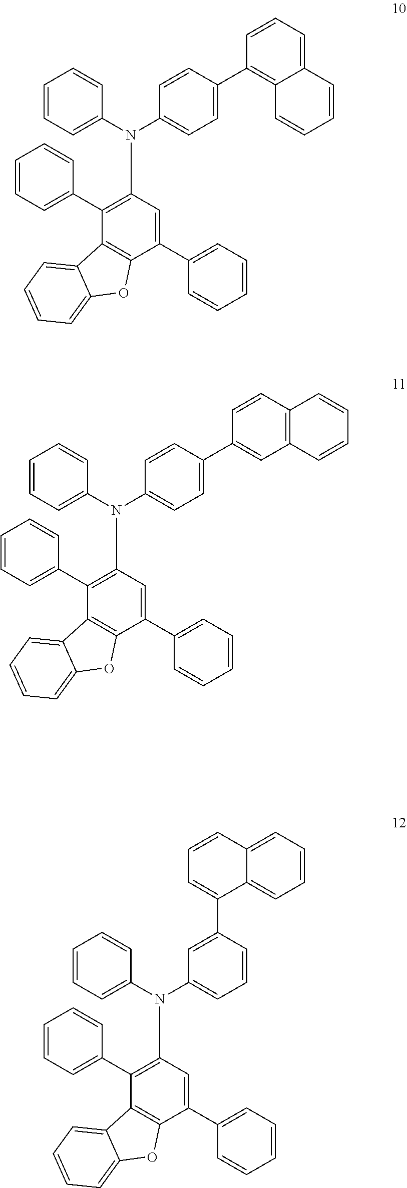 [SMILES: c1ccc(-c2cc(N(c3ccccc3)c3ccc(-c4cccc5ccccc45)cc3)c(-c3ccccc3)c3c2oc2ccccc23)cc1 ; c1ccc(-c2cc(N(c3ccccc3)c3ccc(-c4ccc5ccccc5c4)cc3)c(-c3ccccc3)c3c2oc2ccccc23)cc1 ; c1ccc(-c2cc(N(c3ccccc3)c3cccc(-c4cccc5ccccc45)c3)c(-c3ccccc3)c3c2oc2ccccc23)cc1]

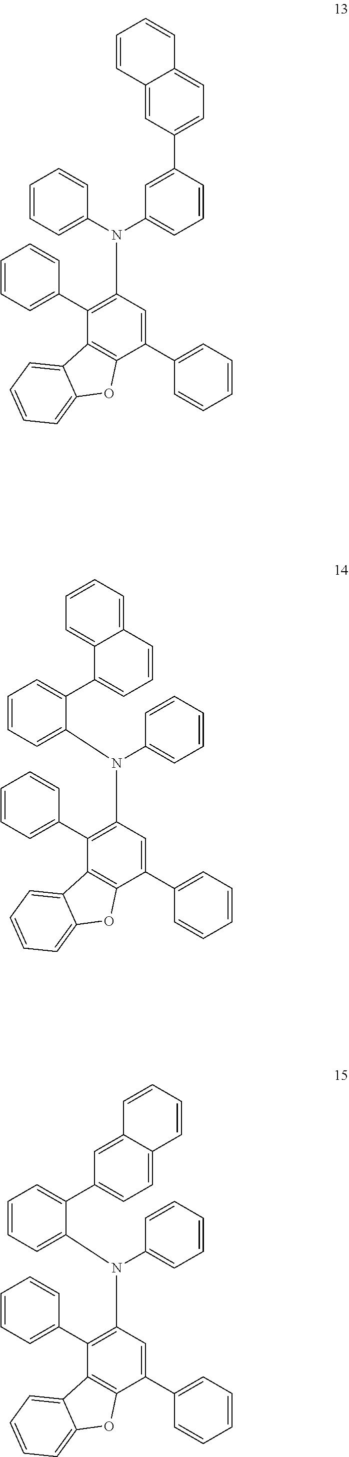 [SMILES: c1ccc(-c2cc(N(c3ccccc3)c3cccc(-c4ccc5ccccc5c4)c3)c(-c3ccccc3)c3c2oc2ccccc23)cc1 ; c1ccc(-c2cc(N(c3ccccc3)c3ccccc3-c3cccc4ccccc34)c(-c3ccccc3)c3c2oc2ccccc23)cc1 ; c1ccc(-c2cc(N(c3ccccc3)c3ccccc3-c3ccc4ccccc4c3)c(-c3ccccc3)c3c2oc2ccccc23)cc1]

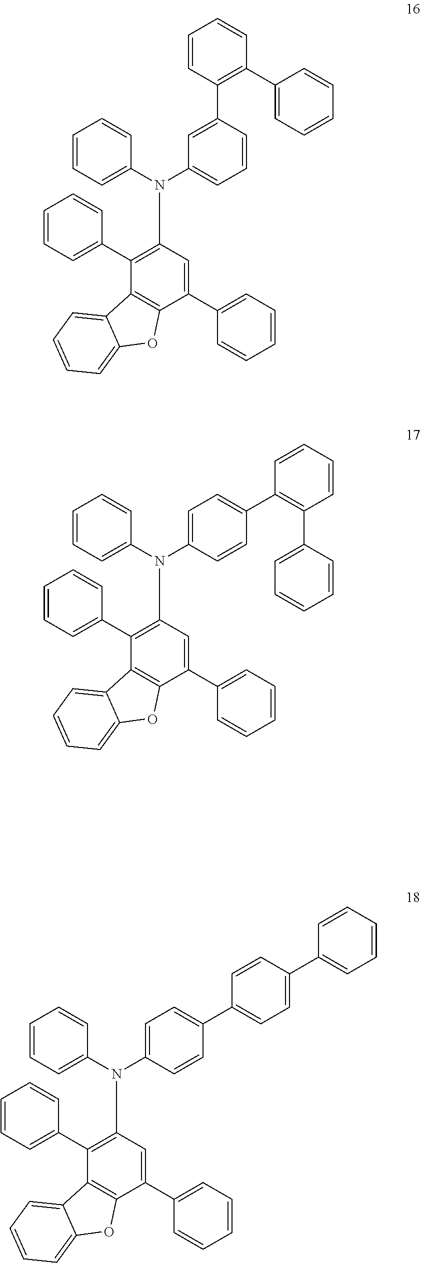 [SMILES: c1ccc(-c2ccccc2-c2cccc(N(c3ccccc3)c3cc(-c4ccccc4)c4oc5ccccc5c4c3-c3ccccc3)c2)cc1 ; c1ccc(-c2ccccc2-c2ccc(N(c3ccccc3)c3cc(-c4ccccc4)c4oc5ccccc5c4c3-c3ccccc3)cc2)cc1 ; c1ccc(-c2ccc(-c3ccc(N(c4ccccc4)c4cc(-c5ccccc5)c5oc6ccccc6c5c4-c4ccccc4)cc3)cc2)cc1]

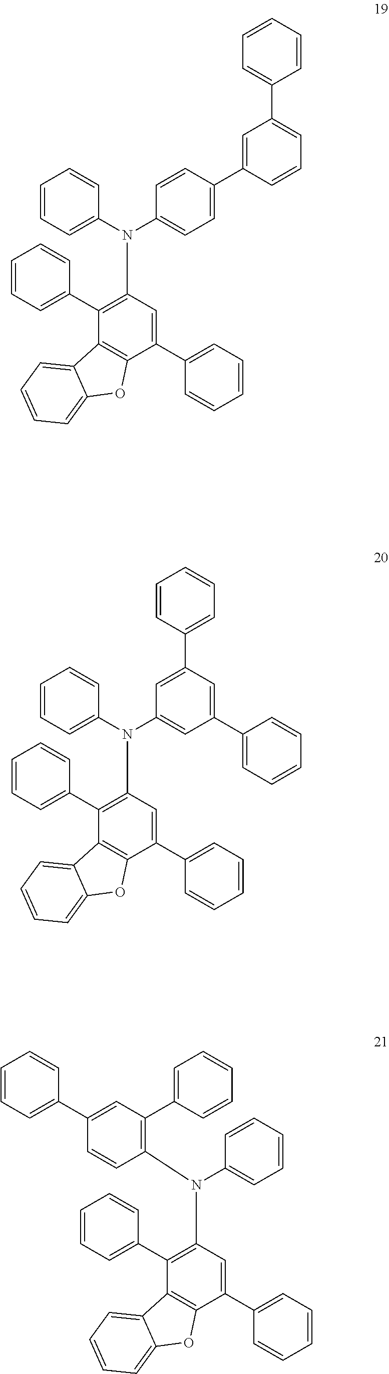 [SMILES: c1ccc(-c2cccc(-c3ccc(N(c4ccccc4)c4cc(-c5ccccc5)c5oc6ccccc6c5c4-c4ccccc4)cc3)c2)cc1 ; c1ccc(-c2cc(-c3ccccc3)cc(N(c3ccccc3)c3cc(-c4ccccc4)c4oc5ccccc5c4c3-c3ccccc3)c2)cc1 ; c1ccc(-c2ccc(N(c3ccccc3)c3cc(-c4ccccc4)c4oc5ccccc5c4c3-c3ccccc3)c(-c3ccccc3)c2)cc1]

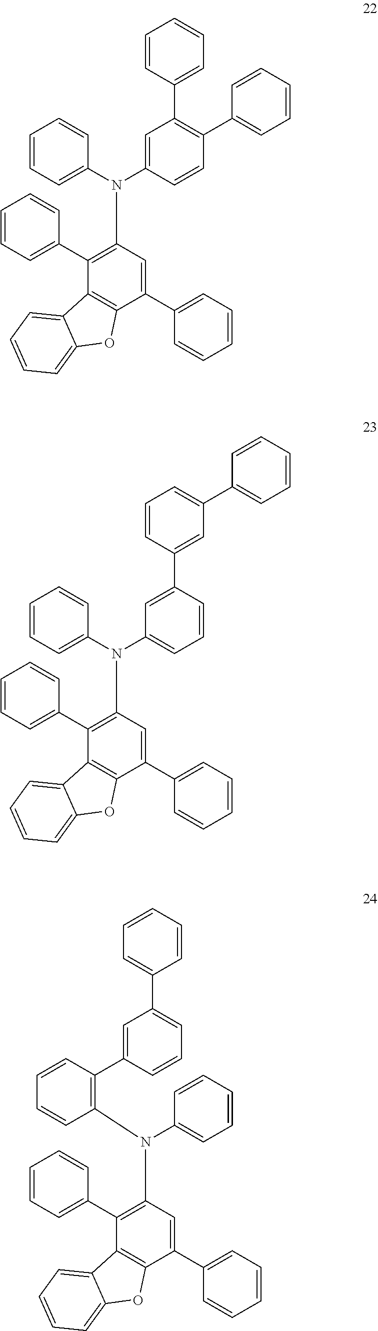 [SMILES: c1ccc(-c2ccc(N(c3ccccc3)c3cc(-c4ccccc4)c4oc5ccccc5c4c3-c3ccccc3)cc2-c2ccccc2)cc1 ; c1ccc(-c2cccc(-c3cccc(N(c4ccccc4)c4cc(-c5ccccc5)c5oc6ccccc6c5c4-c4ccccc4)c3)c2)cc1 ; c1ccc(-c2cccc(-c3ccccc3N(c3ccccc3)c3cc(-c4ccccc4)c4oc5ccccc5c4c3-c3ccccc3)c2)cc1]

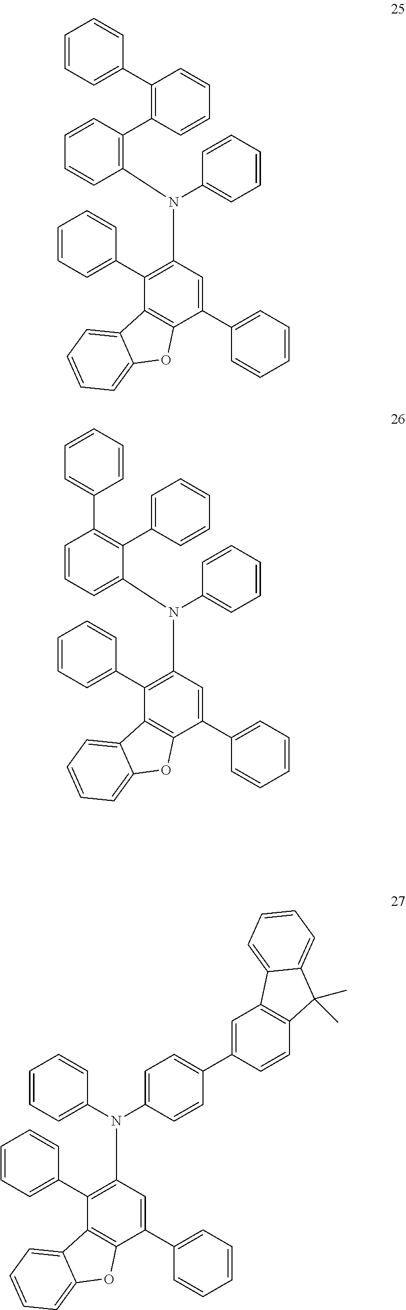

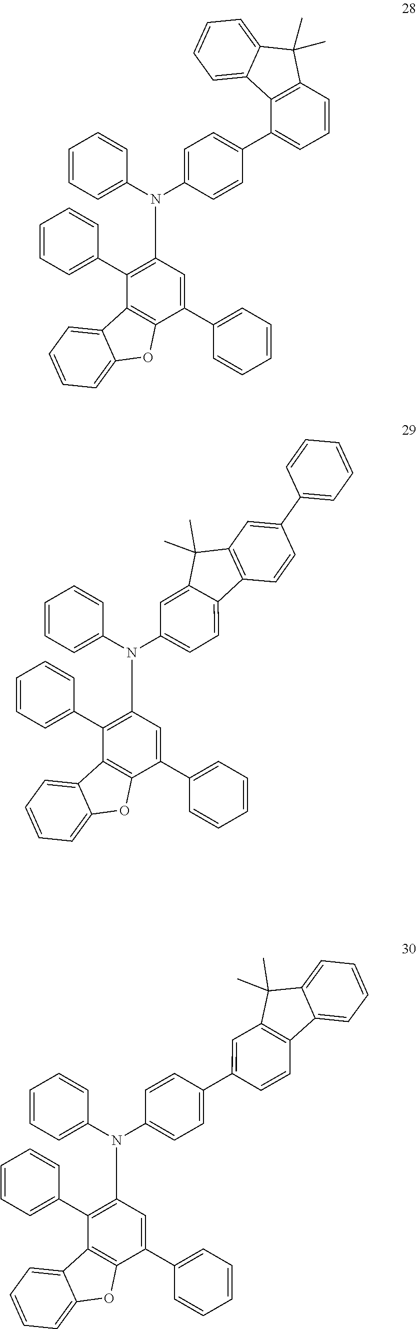 [SMILES: CC1(C)c2ccccc2-c2c(-c3ccc(N(c4ccccc4)c4cc(-c5ccccc5)c5oc6ccccc6c5c4-c4ccccc4)cc3)cccc21 ; CC1(C)c2cc(-c3ccccc3)ccc2-c2ccc(N(c3ccccc3)c3cc(-c4ccccc4)c4oc5ccccc5c4c3-c3ccccc3)cc21 ; CC1(C)c2ccccc2-c2ccc(-c3ccc(N(c4ccccc4)c4cc(-c5ccccc5)c5oc6ccccc6c5c4-c4ccccc4)cc3)cc21]

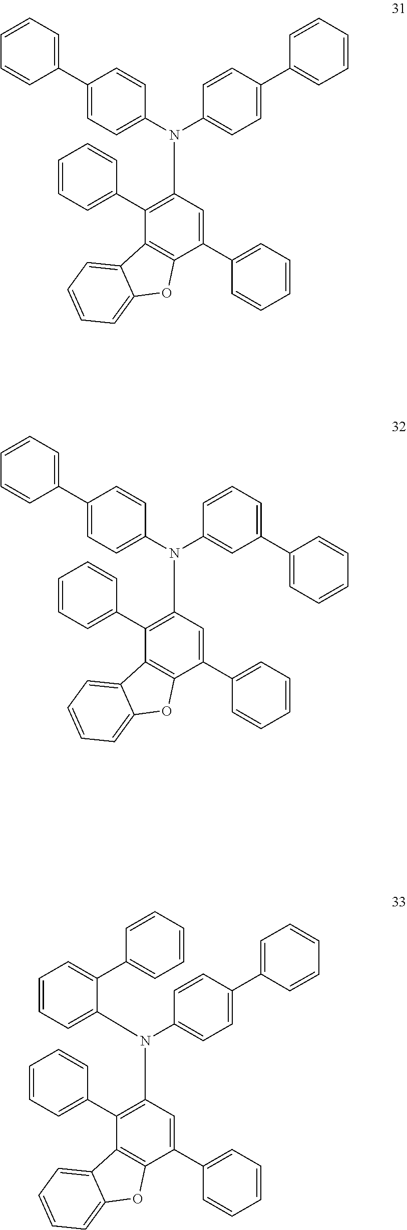 [SMILES: c1ccc(-c2ccc(N(c3ccc(-c4ccccc4)cc3)c3cc(-c4ccccc4)c4oc5ccccc5c4c3-c3ccccc3)cc2)cc1 ; c1ccc(-c2ccc(N(c3cccc(-c4ccccc4)c3)c3cc(-c4ccccc4)c4oc5ccccc5c4c3-c3ccccc3)cc2)cc1 ; c1ccc(-c2ccc(N(c3ccccc3-c3ccccc3)c3cc(-c4ccccc4)c4oc5ccccc5c4c3-c3ccccc3)cc2)cc1]

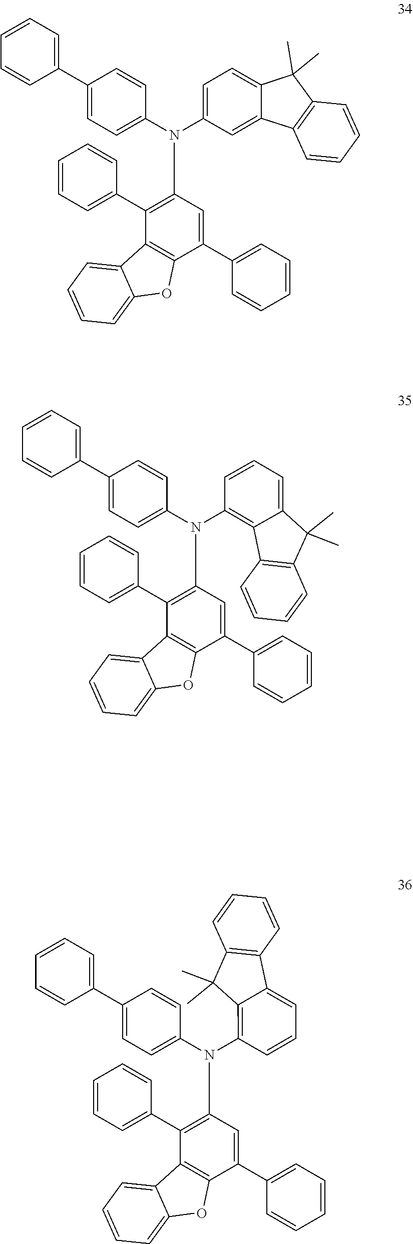 [SMILES: CC1(C)c2ccccc2-c2cc(N(c3ccc(-c4ccccc4)cc3)c3cc(-c4ccccc4)c4oc5ccccc5c4c3-c3ccccc3)ccc21 ; CC1(C)c2ccccc2-c2c(N(c3ccc(-c4ccccc4)cc3)c3cc(-c4ccccc4)c4oc5ccccc5c4c3-c3ccccc3)cccc21 ; CC1(C)c2ccccc2-c2cccc(N(c3ccc(-c4ccccc4)cc3)c3cc(-c4ccccc4)c4oc5ccccc5c4c3-c3ccccc3)c21]

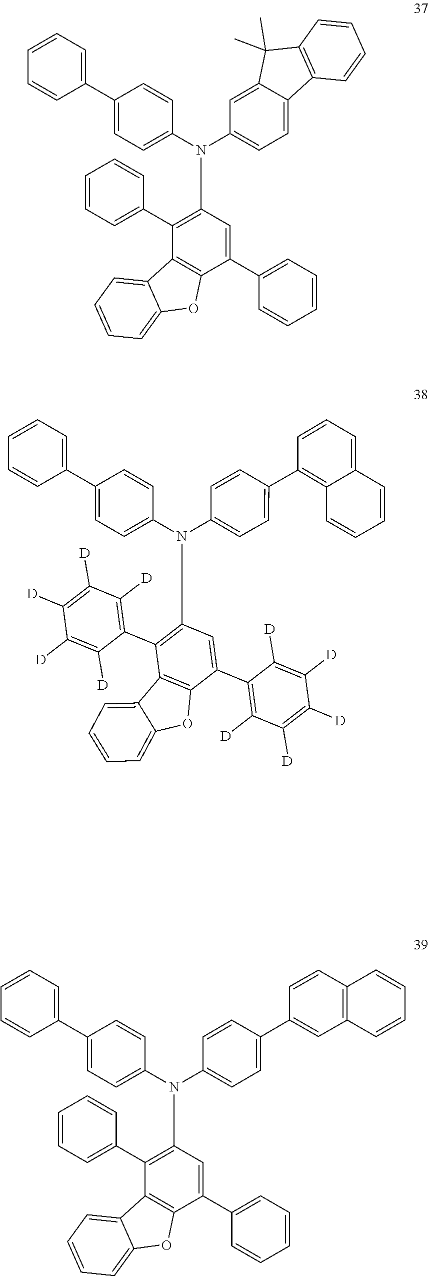 [SMILES: CC1(C)c2ccccc2-c2ccc(N(c3ccc(-c4ccccc4)cc3)c3cc(-c4ccccc4)c4oc5ccccc5c4c3-c3ccccc3)cc21 ; [2H]c1c([2H])c([2H])c(-c2cc(N(c3ccc(-c4ccccc4)cc3)c3ccc(-c4cccc5ccccc45)cc3)c(-c3c([2H])c([2H])c([2H])c([2H])c3[2H])c3c2oc2ccccc23)c([2H])c1[2H] ; c1ccc(-c2ccc(N(c3ccc(-c4ccc5ccccc5c4)cc3)c3cc(-c4ccccc4)c4oc5ccccc5c4c3-c3ccccc3)cc2)cc1]

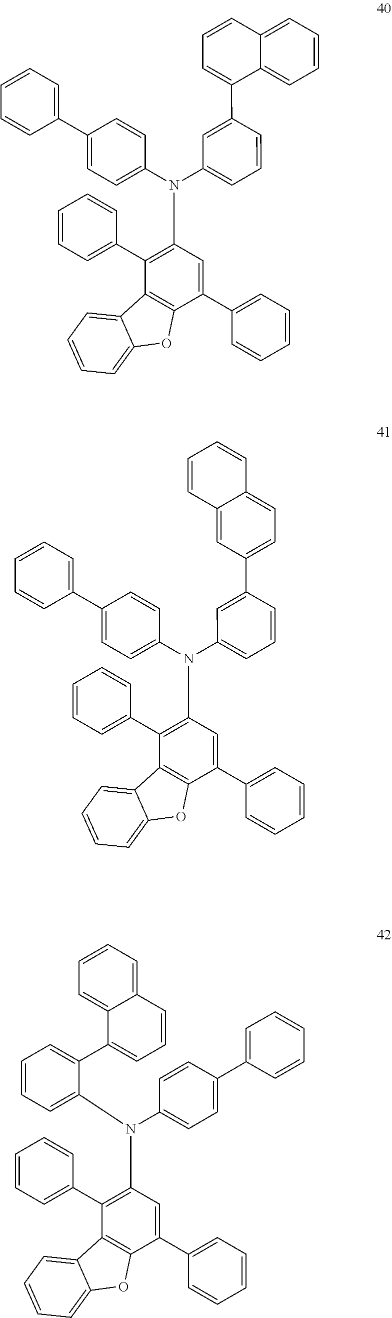 [SMILES: c1ccc(-c2ccc(N(c3cccc(-c4cccc5ccccc45)c3)c3cc(-c4ccccc4)c4oc5ccccc5c4c3-c3ccccc3)cc2)cc1 ; c1ccc(-c2ccc(N(c3cccc(-c4ccc5ccccc5c4)c3)c3cc(-c4ccccc4)c4oc5ccccc5c4c3-c3ccccc3)cc2)cc1 ; c1ccc(-c2ccc(N(c3ccccc3-c3cccc4ccccc34)c3cc(-c4ccccc4)c4oc5ccccc5c4c3-c3ccccc3)cc2)cc1]

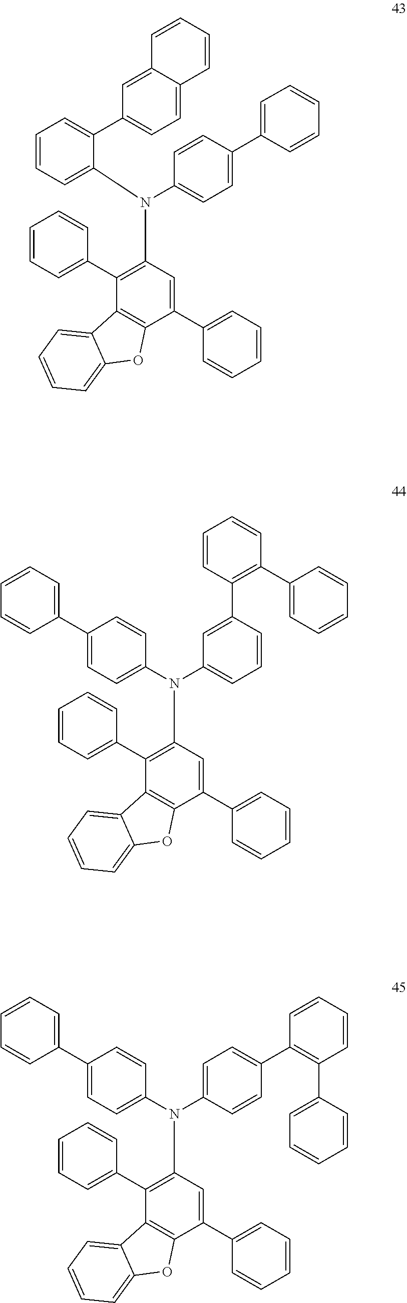 [SMILES: c1ccc(-c2ccc(N(c3ccccc3-c3ccc4ccccc4c3)c3cc(-c4ccccc4)c4oc5ccccc5c4c3-c3ccccc3)cc2)cc1 ; c1ccc(-c2ccc(N(c3cccc(-c4ccccc4-c4ccccc4)c3)c3cc(-c4ccccc4)c4oc5ccccc5c4c3-c3ccccc3)cc2)cc1 ; c1ccc(-c2ccc(N(c3ccc(-c4ccccc4-c4ccccc4)cc3)c3cc(-c4ccccc4)c4oc5ccccc5c4c3-c3ccccc3)cc2)cc1]

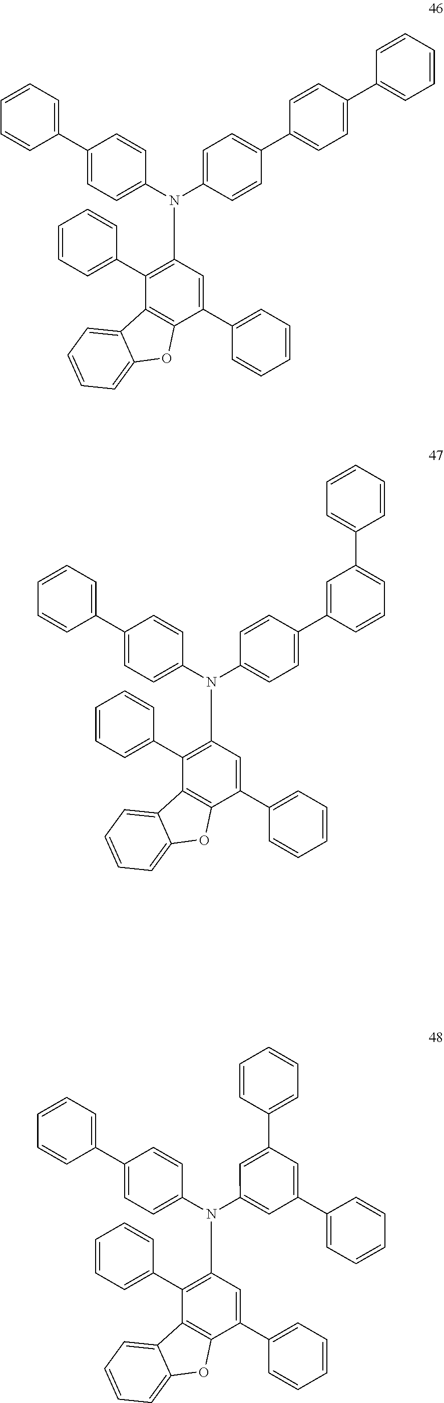 [SMILES: c1ccc(-c2ccc(-c3ccc(N(c4ccc(-c5ccccc5)cc4)c4cc(-c5ccccc5)c5oc6ccccc6c5c4-c4ccccc4)cc3)cc2)cc1 ; c1ccc(-c2ccc(N(c3ccc(-c4cccc(-c5ccccc5)c4)cc3)c3cc(-c4ccccc4)c4oc5ccccc5c4c3-c3ccccc3)cc2)cc1 ; c1ccc(-c2ccc(N(c3cc(-c4ccccc4)cc(-c4ccccc4)c3)c3cc(-c4ccccc4)c4oc5ccccc5c4c3-c3ccccc3)cc2)cc1]

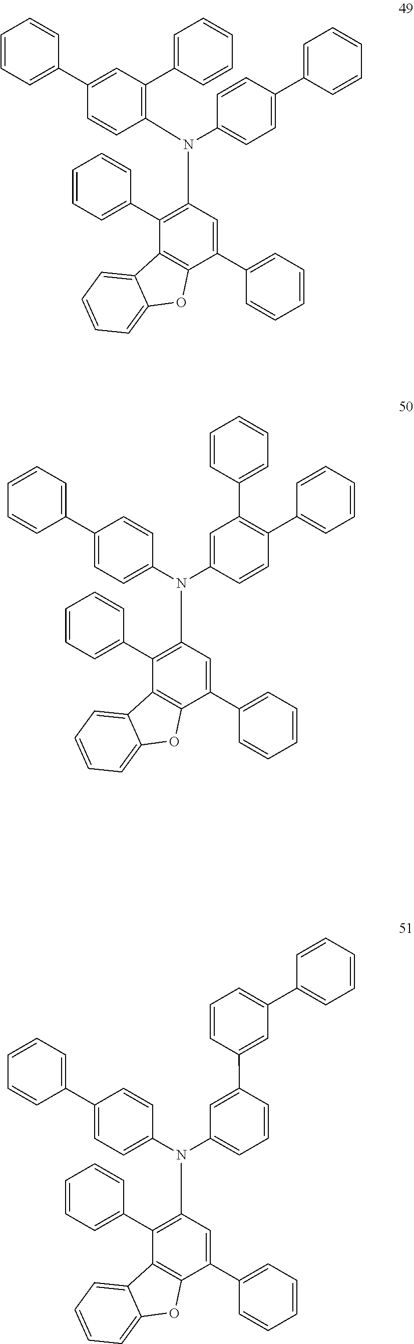 [SMILES: c1ccc(-c2ccc(N(c3ccc(-c4ccccc4)cc3-c3ccccc3)c3cc(-c4ccccc4)c4oc5ccccc5c4c3-c3ccccc3)cc2)cc1 ; c1ccc(-c2ccc(N(c3ccc(-c4ccccc4)c(-c4ccccc4)c3)c3cc(-c4ccccc4)c4oc5ccccc5c4c3-c3ccccc3)cc2)cc1 ; c1ccc(-c2ccc(N(c3cccc(-c4cccc(-c5ccccc5)c4)c3)c3cc(-c4ccccc4)c4oc5ccccc5c4c3-c3ccccc3)cc2)cc1]

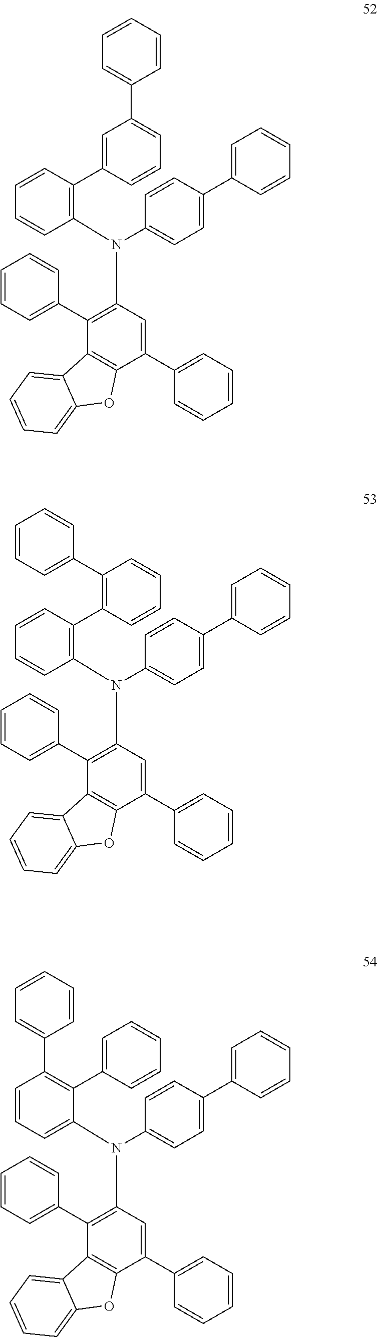 [SMILES: c1ccc(-c2ccc(N(c3ccccc3-c3cccc(-c4ccccc4)c3)c3cc(-c4ccccc4)c4oc5ccccc5c4c3-c3ccccc3)cc2)cc1 ; c1ccc(-c2ccc(N(c3ccccc3-c3ccccc3-c3ccccc3)c3cc(-c4ccccc4)c4oc5ccccc5c4c3-c3ccccc3)cc2)cc1 ; c1ccc(-c2ccc(N(c3cccc(-c4ccccc4)c3-c3ccccc3)c3cc(-c4ccccc4)c4oc5ccccc5c4c3-c3ccccc3)cc2)cc1]

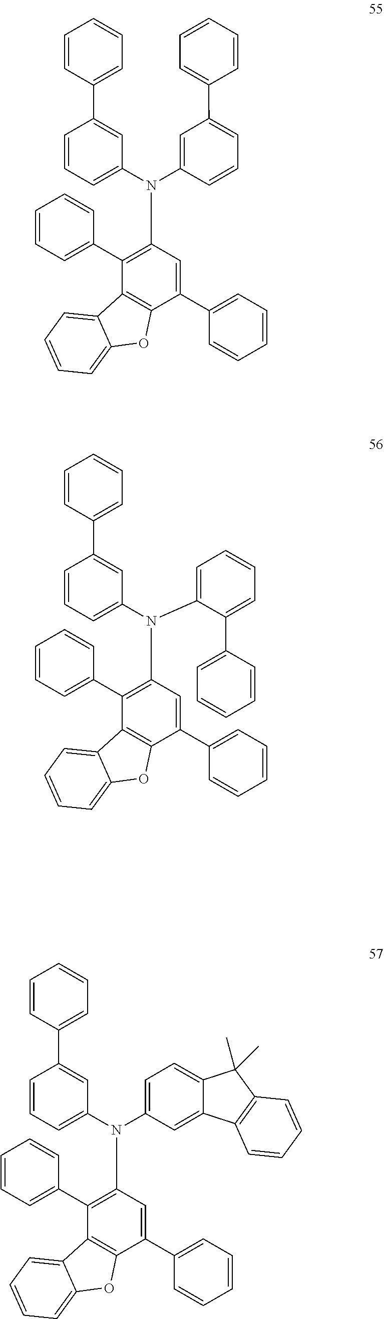 [SMILES: c1ccc(-c2cccc(N(c3cccc(-c4ccccc4)c3)c3cc(-c4ccccc4)c4oc5ccccc5c4c3-c3ccccc3)c2)cc1 ; c1ccc(-c2cccc(N(c3ccccc3-c3ccccc3)c3cc(-c4ccccc4)c4oc5ccccc5c4c3-c3ccccc3)c2)cc1 ; CC1(C)c2ccccc2-c2cc(N(c3cccc(-c4ccccc4)c3)c3cc(-c4ccccc4)c4oc5ccccc5c4c3-c3ccccc3)ccc21]

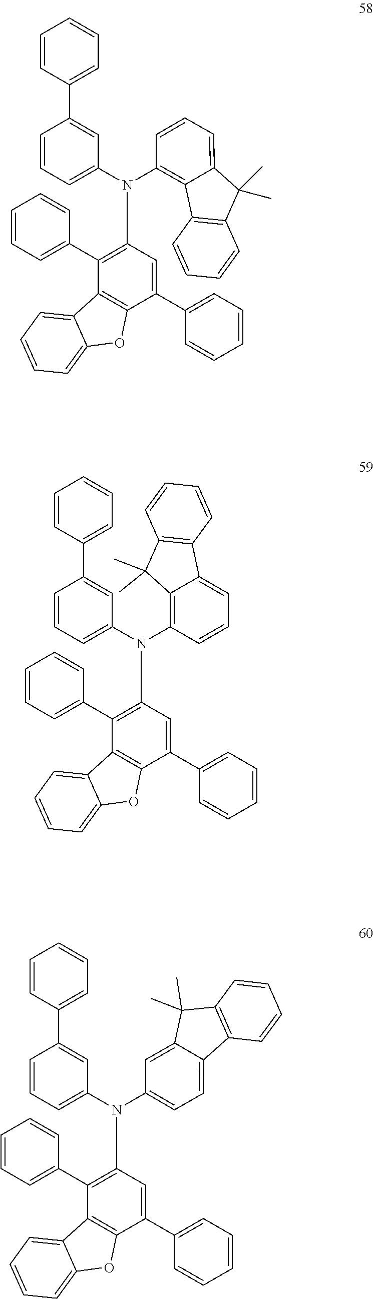 [SMILES: CC1(C)c2ccccc2-c2c(N(c3cccc(-c4ccccc4)c3)c3cc(-c4ccccc4)c4oc5ccccc5c4c3-c3ccccc3)cccc21 ; CC1(C)c2ccccc2-c2cccc(N(c3cccc(-c4ccccc4)c3)c3cc(-c4ccccc4)c4oc5ccccc5c4c3-c3ccccc3)c21 ; CC1(C)c2ccccc2-c2ccc(N(c3cccc(-c4ccccc4)c3)c3cc(-c4ccccc4)c4oc5ccccc5c4c3-c3ccccc3)cc21]

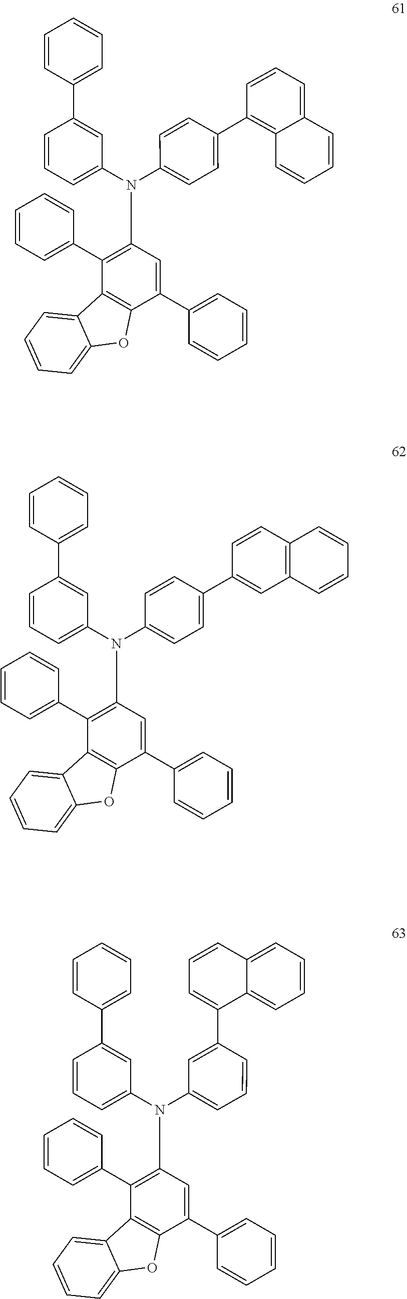 [SMILES: c1ccc(-c2cccc(N(c3ccc(-c4cccc5ccccc45)cc3)c3cc(-c4ccccc4)c4oc5ccccc5c4c3-c3ccccc3)c2)cc1 ; c1ccc(-c2cccc(N(c3ccc(-c4ccc5ccccc5c4)cc3)c3cc(-c4ccccc4)c4oc5ccccc5c4c3-c3ccccc3)c2)cc1 ; c1ccc(-c2cccc(N(c3cccc(-c4cccc5ccccc45)c3)c3cc(-c4ccccc4)c4oc5ccccc5c4c3-c3ccccc3)c2)cc1]

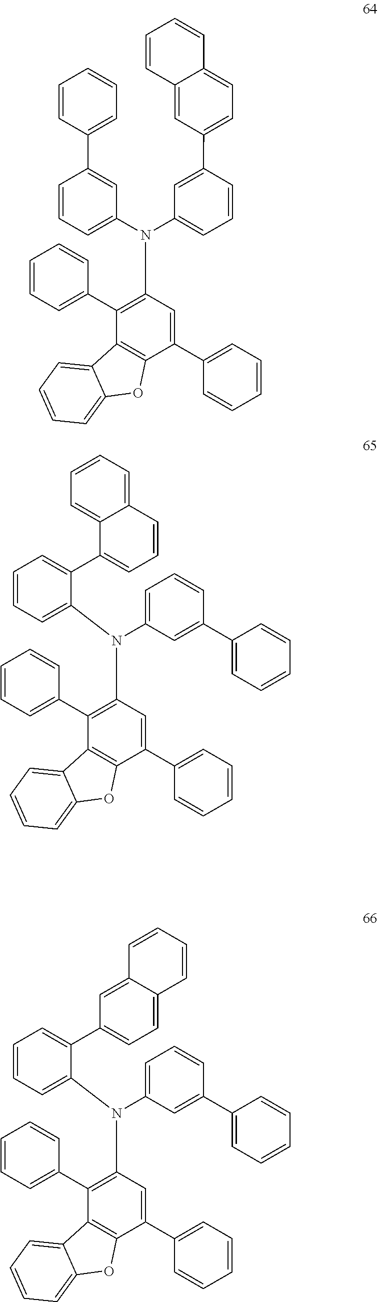

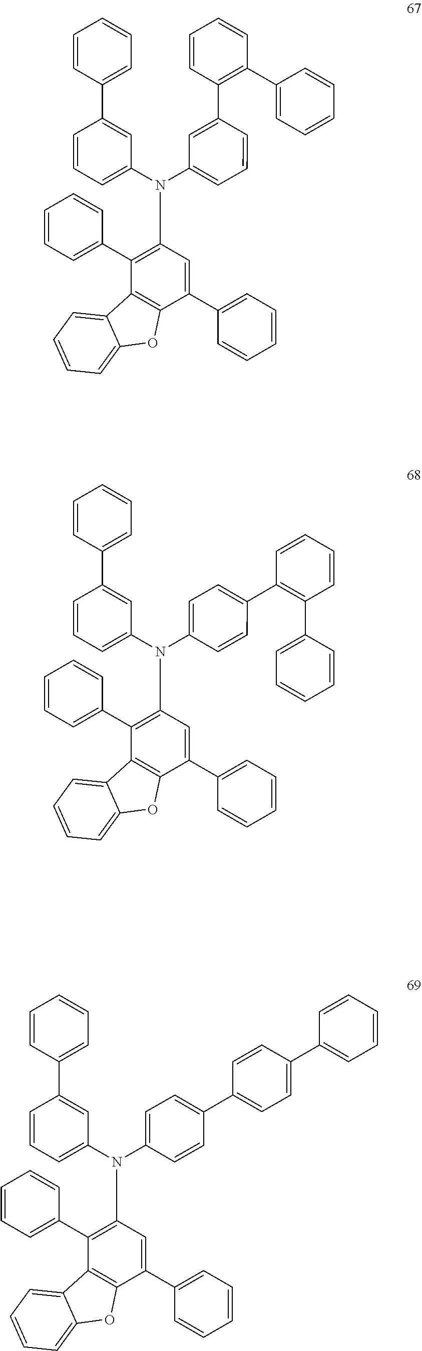 [SMILES: c1ccc(-c2cccc(N(c3cccc(-c4ccccc4-c4ccccc4)c3)c3cc(-c4ccccc4)c4oc5ccccc5c4c3-c3ccccc3)c2)cc1 ; c1ccc(-c2cccc(N(c3ccc(-c4ccccc4-c4ccccc4)cc3)c3cc(-c4ccccc4)c4oc5ccccc5c4c3-c3ccccc3)c2)cc1 ; c1ccc(-c2ccc(-c3ccc(N(c4cccc(-c5ccccc5)c4)c4cc(-c5ccccc5)c5oc6ccccc6c5c4-c4ccccc4)cc3)cc2)cc1]

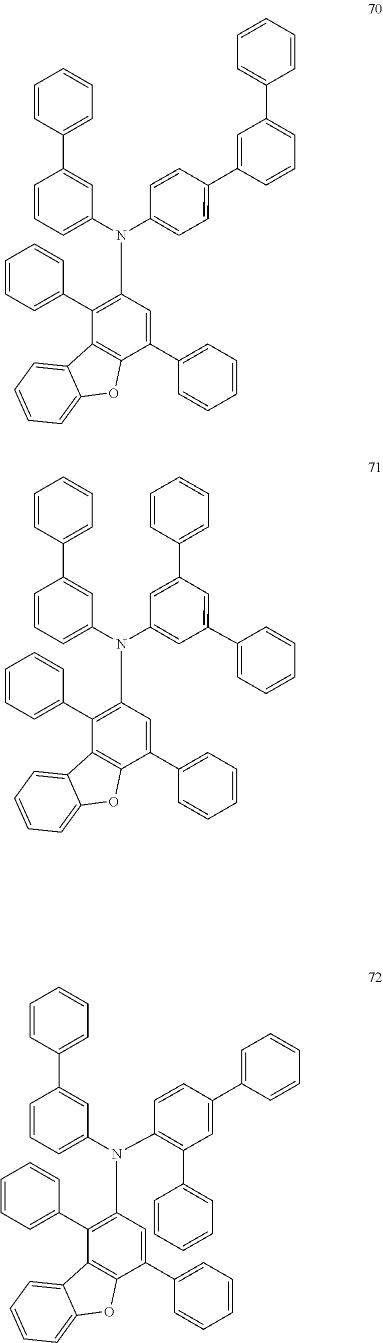 [SMILES: c1ccc(-c2cccc(-c3ccc(N(c4cccc(-c5ccccc5)c4)c4cc(-c5ccccc5)c5oc6ccccc6c5c4-c4ccccc4)cc3)c2)cc1 ; c1ccc(-c2cccc(N(c3cc(-c4ccccc4)cc(-c4ccccc4)c3)c3cc(-c4ccccc4)c4oc5ccccc5c4c3-c3ccccc3)c2)cc1 ; c1ccc(-c2cccc(N(c3ccc(-c4ccccc4)cc3-c3ccccc3)c3cc(-c4ccccc4)c4oc5ccccc5c4c3-c3ccccc3)c2)cc1]

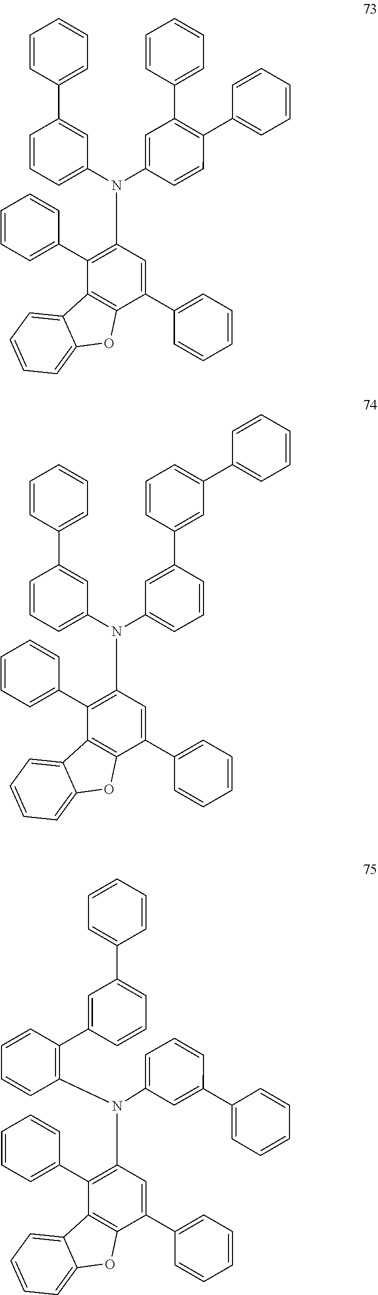 [SMILES: c1ccc(-c2cccc(N(c3ccc(-c4ccccc4)c(-c4ccccc4)c3)c3cc(-c4ccccc4)c4oc5ccccc5c4c3-c3ccccc3)c2)cc1 ; c1ccc(-c2cccc(-c3cccc(N(c4cccc(-c5ccccc5)c4)c4cc(-c5ccccc5)c5oc6ccccc6c5c4-c4ccccc4)c3)c2)cc1 ; c1ccc(-c2cccc(-c3ccccc3N(c3cccc(-c4ccccc4)c3)c3cc(-c4ccccc4)c4oc5ccccc5c4c3-c3ccccc3)c2)cc1]

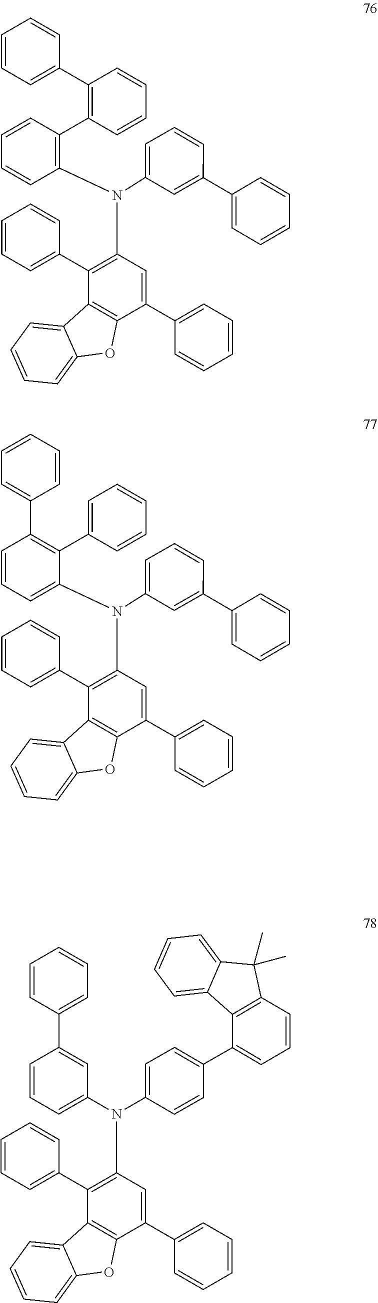 [SMILES: c1ccc(-c2cccc(N(c3ccccc3-c3ccccc3-c3ccccc3)c3cc(-c4ccccc4)c4oc5ccccc5c4c3-c3ccccc3)c2)cc1 ; c1ccc(-c2cccc(N(c3cccc(-c4ccccc4)c3-c3ccccc3)c3cc(-c4ccccc4)c4oc5ccccc5c4c3-c3ccccc3)c2)cc1 ; CC1(C)c2ccccc2-c2c(-c3ccc(N(c4cccc(-c5ccccc5)c4)c4cc(-c5ccccc5)c5oc6ccccc6c5c4-c4ccccc4)cc3)cccc21]

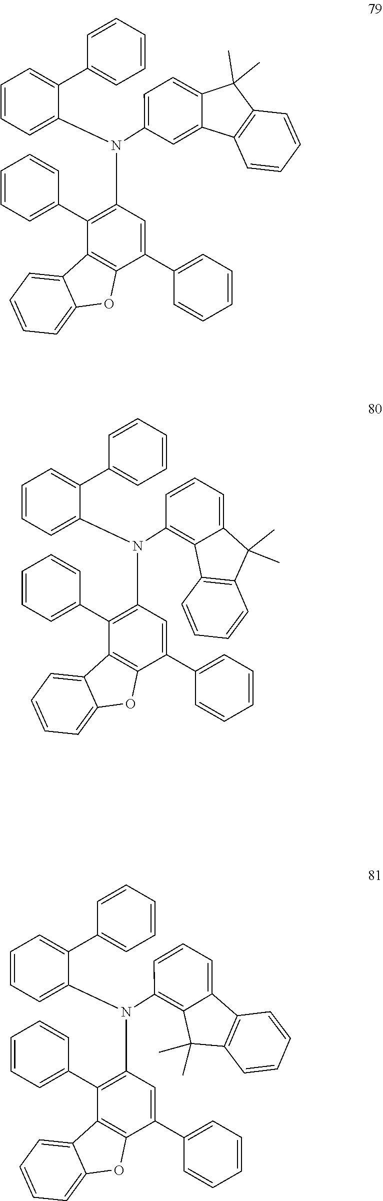 [SMILES: CC1(C)c2ccccc2-c2cc(N(c3ccccc3-c3ccccc3)c3cc(-c4ccccc4)c4oc5ccccc5c4c3-c3ccccc3)ccc21 ; CC1(C)c2ccccc2-c2c(N(c3ccccc3-c3ccccc3)c3cc(-c4ccccc4)c4oc5ccccc5c4c3-c3ccccc3)cccc21 ; CC1(C)c2ccccc2-c2cccc(N(c3ccccc3-c3ccccc3)c3cc(-c4ccccc4)c4oc5ccccc5c4c3-c3ccccc3)c21]

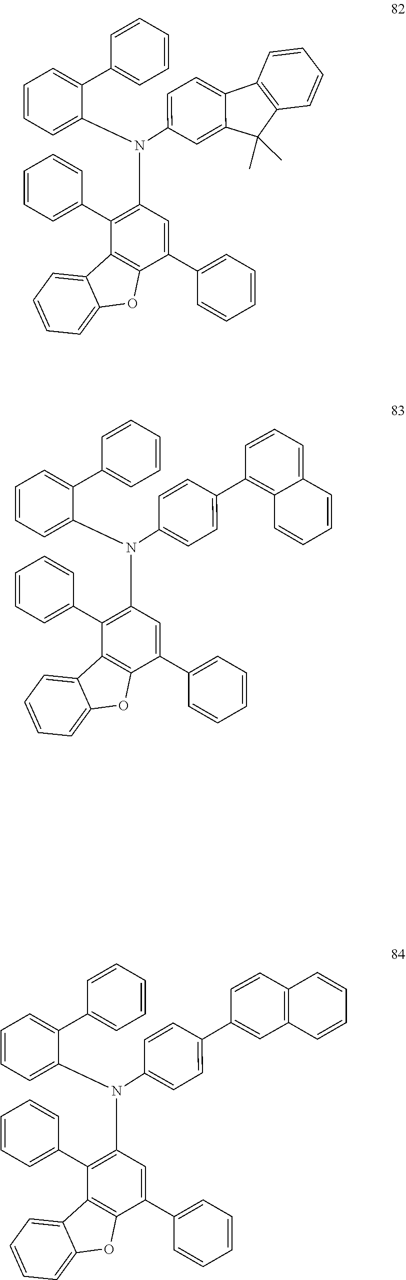 [SMILES: CC1(C)c2ccccc2-c2ccc(N(c3ccccc3-c3ccccc3)c3cc(-c4ccccc4)c4oc5ccccc5c4c3-c3ccccc3)cc21 ; c1ccc(-c2ccccc2N(c2ccc(-c3cccc4ccccc34)cc2)c2cc(-c3ccccc3)c3oc4ccccc4c3c2-c2ccccc2)cc1 ; c1ccc(-c2ccccc2N(c2ccc(-c3ccc4ccccc4c3)cc2)c2cc(-c3ccccc3)c3oc4ccccc4c3c2-c2ccccc2)cc1]

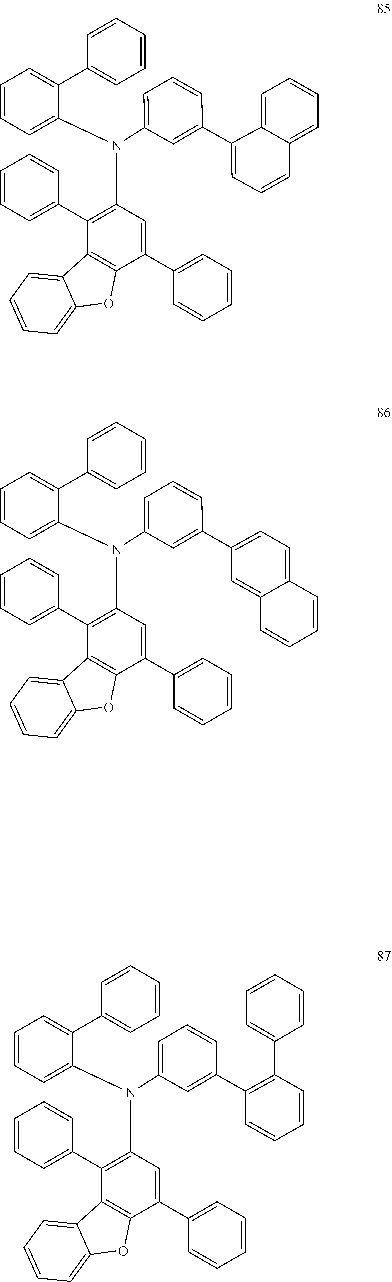 [SMILES: c1ccc(-c2ccccc2N(c2cccc(-c3cccc4ccccc34)c2)c2cc(-c3ccccc3)c3oc4ccccc4c3c2-c2ccccc2)cc1 ; c1ccc(-c2ccccc2N(c2cccc(-c3ccc4ccccc4c3)c2)c2cc(-c3ccccc3)c3oc4ccccc4c3c2-c2ccccc2)cc1 ; c1ccc(-c2ccccc2-c2cccc(N(c3ccccc3-c3ccccc3)c3cc(-c4ccccc4)c4oc5ccccc5c4c3-c3ccccc3)c2)cc1]

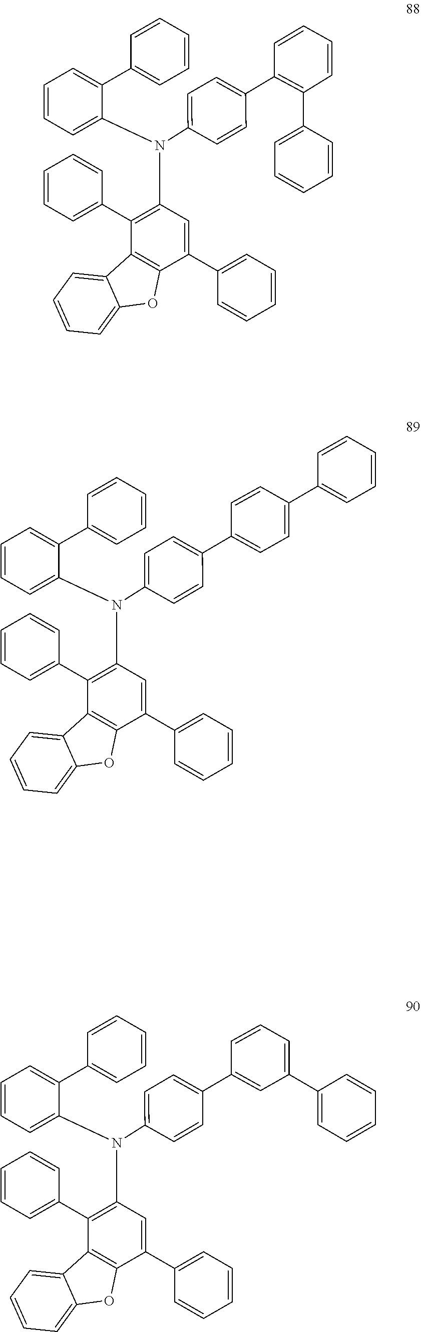 [SMILES: c1ccc(-c2ccccc2-c2ccc(N(c3ccccc3-c3ccccc3)c3cc(-c4ccccc4)c4oc5ccccc5c4c3-c3ccccc3)cc2)cc1 ; c1ccc(-c2ccc(-c3ccc(N(c4ccccc4-c4ccccc4)c4cc(-c5ccccc5)c5oc6ccccc6c5c4-c4ccccc4)cc3)cc2)cc1 ; c1ccc(-c2cccc(-c3ccc(N(c4ccccc4-c4ccccc4)c4cc(-c5ccccc5)c5oc6ccccc6c5c4-c4ccccc4)cc3)c2)cc1]

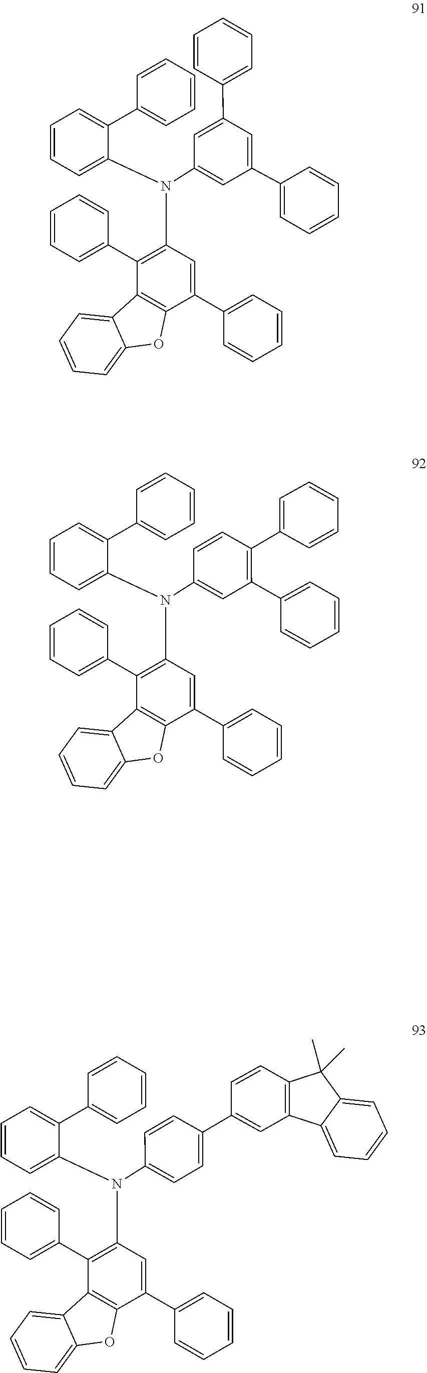 [SMILES: c1ccc(-c2cc(-c3ccccc3)cc(N(c3ccccc3-c3ccccc3)c3cc(-c4ccccc4)c4oc5ccccc5c4c3-c3ccccc3)c2)cc1 ; c1ccc(-c2ccc(N(c3ccccc3-c3ccccc3)c3cc(-c4ccccc4)c4oc5ccccc5c4c3-c3ccccc3)cc2-c2ccccc2)cc1 ; CC1(C)c2ccccc2-c2cc(-c3ccc(N(c4ccccc4-c4ccccc4)c4cc(-c5ccccc5)c5oc6ccccc6c5c4-c4ccccc4)cc3)ccc21]

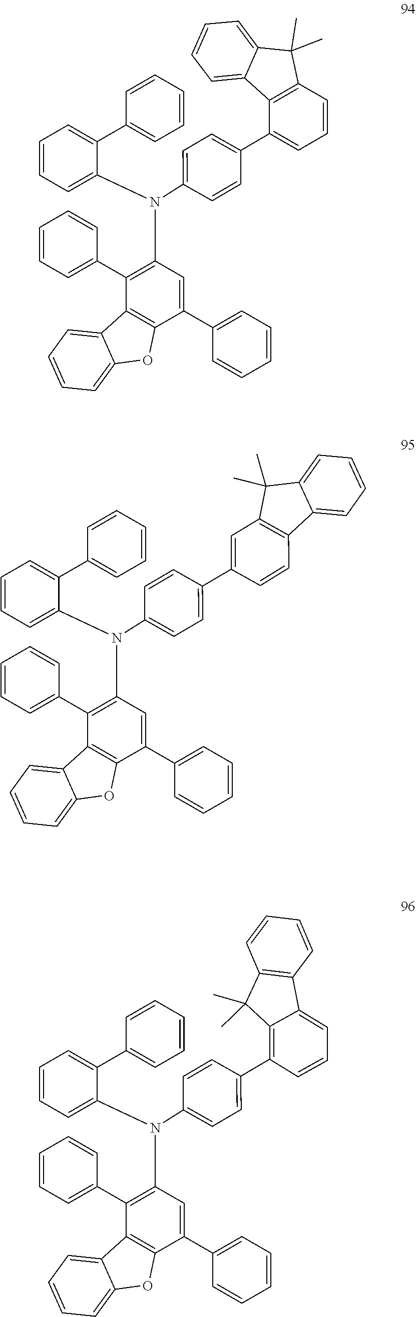 [SMILES: CC1(C)c2ccccc2-c2c(-c3ccc(N(c4ccccc4-c4ccccc4)c4cc(-c5ccccc5)c5oc6ccccc6c5c4-c4ccccc4)cc3)cccc21 ; CC1(C)c2ccccc2-c2ccc(-c3ccc(N(c4ccccc4-c4ccccc4)c4cc(-c5ccccc5)c5oc6ccccc6c5c4-c4ccccc4)cc3)cc21 ; CC1(C)c2ccccc2-c2cccc(-c3ccc(N(c4ccccc4-c4ccccc4)c4cc(-c5ccccc5)c5oc6ccccc6c5c4-c4ccccc4)cc3)c21]

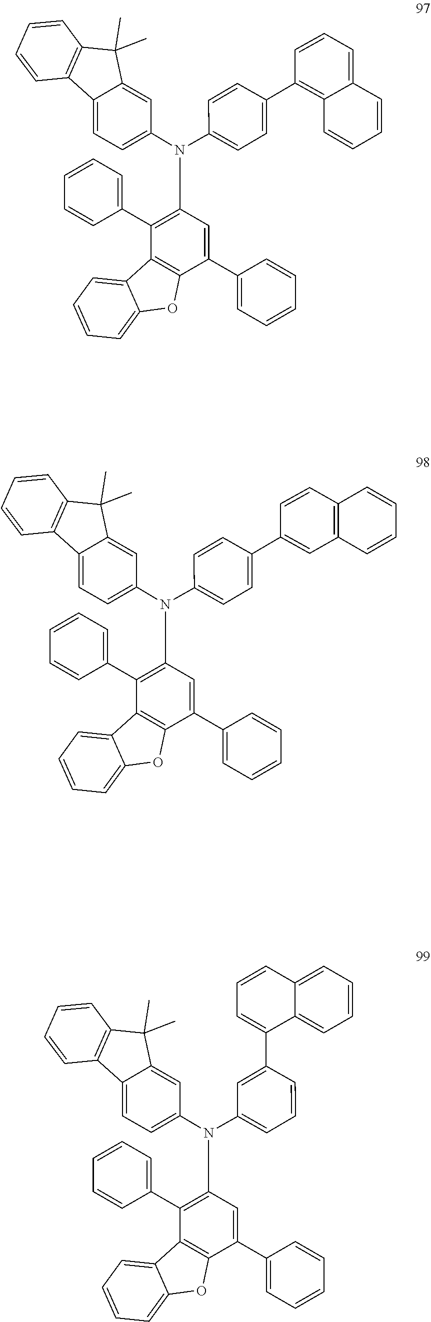 [SMILES: CC1(C)c2ccccc2-c2ccc(N(c3ccc(-c4cccc5ccccc45)cc3)c3cc(-c4ccccc4)c4oc5ccccc5c4c3-c3ccccc3)cc21 ; CC1(C)c2ccccc2-c2ccc(N(c3ccc(-c4ccc5ccccc5c4)cc3)c3cc(-c4ccccc4)c4oc5ccccc5c4c3-c3ccccc3)cc21 ; CC1(C)c2ccccc2-c2ccc(N(c3cccc(-c4cccc5ccccc45)c3)c3cc(-c4ccccc4)c4oc5ccccc5c4c3-c3ccccc3)cc21]

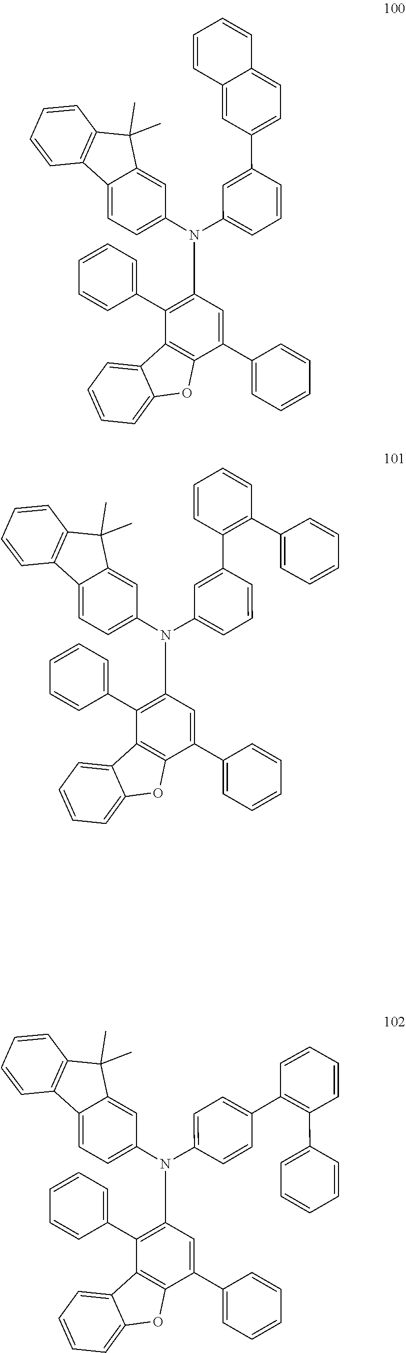 [SMILES: CC1(C)c2ccccc2-c2ccc(N(c3cccc(-c4ccc5ccccc5c4)c3)c3cc(-c4ccccc4)c4oc5ccccc5c4c3-c3ccccc3)cc21 ; CC1(C)c2ccccc2-c2ccc(N(c3cccc(-c4ccccc4-c4ccccc4)c3)c3cc(-c4ccccc4)c4oc5ccccc5c4c3-c3ccccc3)cc21 ; CC1(C)c2ccccc2-c2ccc(N(c3ccc(-c4ccccc4-c4ccccc4)cc3)c3cc(-c4ccccc4)c4oc5ccccc5c4c3-c3ccccc3)cc21]

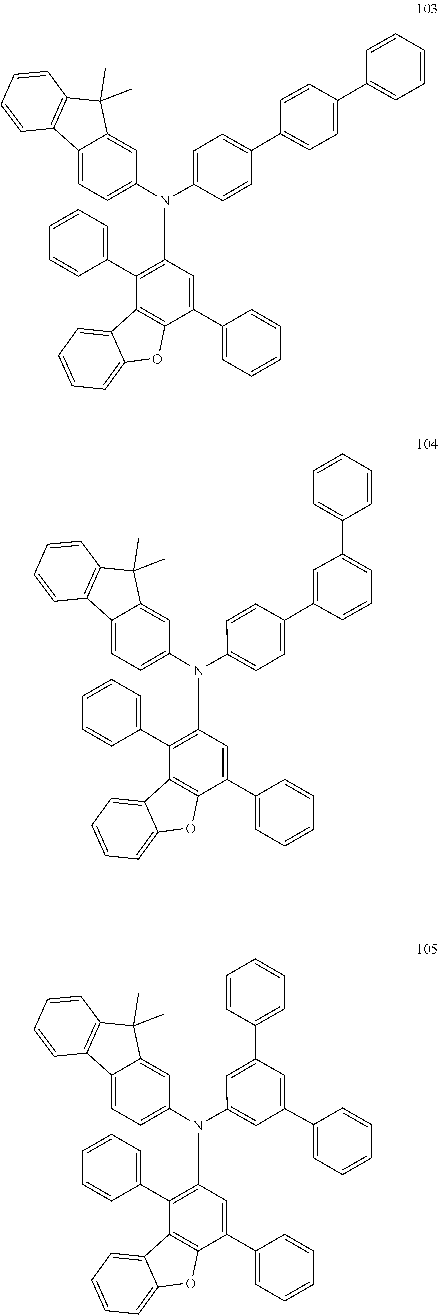 [SMILES: CC1(C)c2ccccc2-c2ccc(N(c3ccc(-c4ccc(-c5ccccc5)cc4)cc3)c3cc(-c4ccccc4)c4oc5ccccc5c4c3-c3ccccc3)cc21 ; CC1(C)c2ccccc2-c2ccc(N(c3ccc(-c4cccc(-c5ccccc5)c4)cc3)c3cc(-c4ccccc4)c4oc5ccccc5c4c3-c3ccccc3)cc21 ; CC1(C)c2ccccc2-c2ccc(N(c3cc(-c4ccccc4)cc(-c4ccccc4)c3)c3cc(-c4ccccc4)c4oc5ccccc5c4c3-c3ccccc3)cc21]

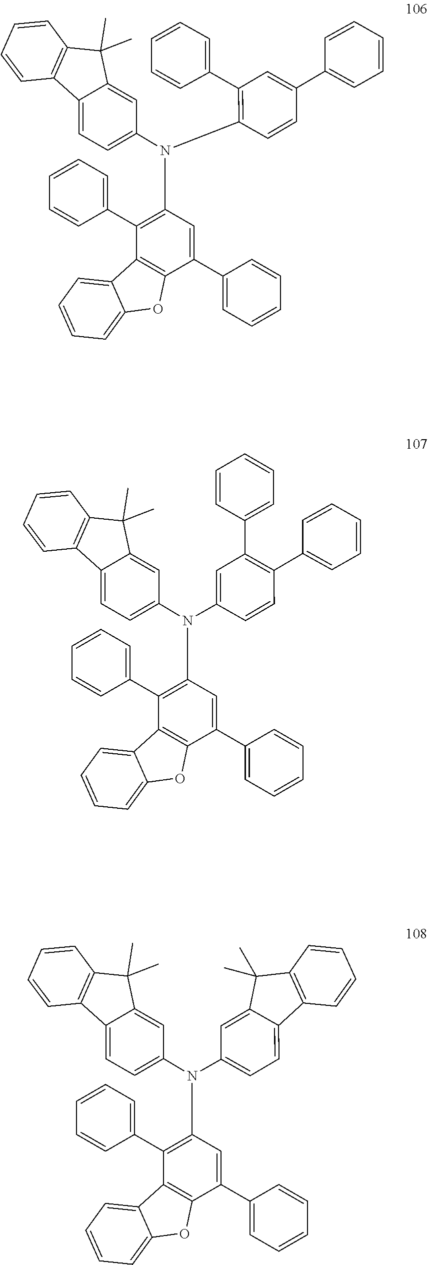 [SMILES: CC1(C)c2ccccc2-c2ccc(N(c3ccc(-c4ccccc4)cc3-c3ccccc3)c3cc(-c4ccccc4)c4oc5ccccc5c4c3-c3ccccc3)cc21 ; CC1(C)c2ccccc2-c2ccc(N(c3ccc(-c4ccccc4)c(-c4ccccc4)c3)c3cc(-c4ccccc4)c4oc5ccccc5c4c3-c3ccccc3)cc21 ; CC1(C)c2ccccc2-c2ccc(N(c3ccc4c(c3)C(C)(C)c3ccccc3-4)c3cc(-c4ccccc4)c4oc5ccccc5c4c3-c3ccccc3)cc21]

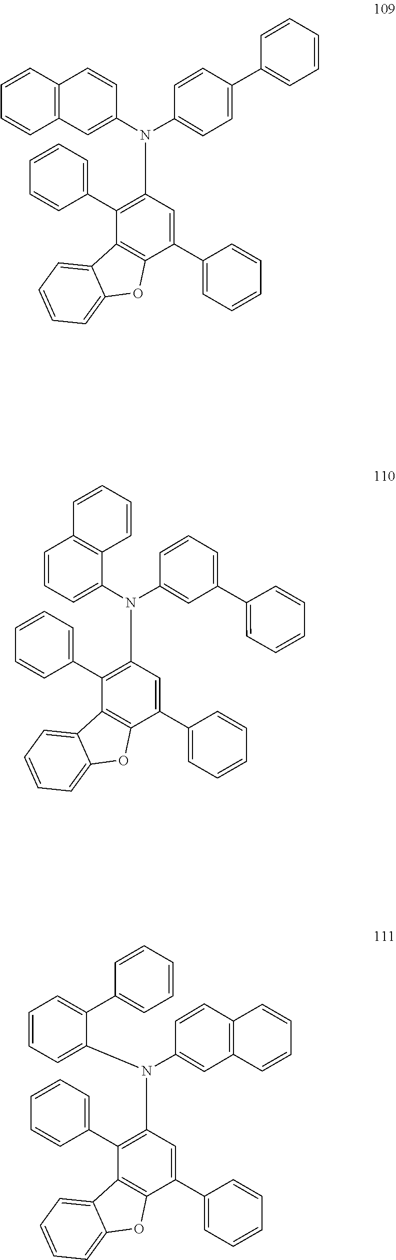 [SMILES: c1ccc(-c2ccc(N(c3ccc4ccccc4c3)c3cc(-c4ccccc4)c4oc5ccccc5c4c3-c3ccccc3)cc2)cc1 ; c1ccc(-c2cccc(N(c3cc(-c4ccccc4)c4oc5ccccc5c4c3-c3ccccc3)c3cccc4ccccc34)c2)cc1 ; c1ccc(-c2ccccc2N(c2ccc3ccccc3c2)c2cc(-c3ccccc3)c3oc4ccccc4c3c2-c2ccccc2)cc1]

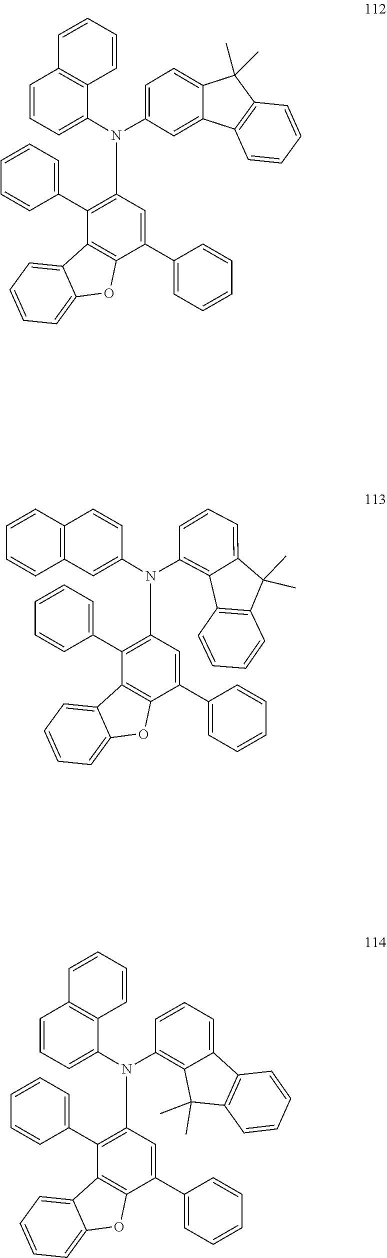 [SMILES: CC1(C)c2ccccc2-c2cc(N(c3cc(-c4ccccc4)c4oc5ccccc5c4c3-c3ccccc3)c3cccc4ccccc34)ccc21 ; CC1(C)c2ccccc2-c2c(N(c3ccc4ccccc4c3)c3cc(-c4ccccc4)c4oc5ccccc5c4c3-c3ccccc3)cccc21 ; CC1(C)c2ccccc2-c2cccc(N(c3cc(-c4ccccc4)c4oc5ccccc5c4c3-c3ccccc3)c3cccc4ccccc34)c21]

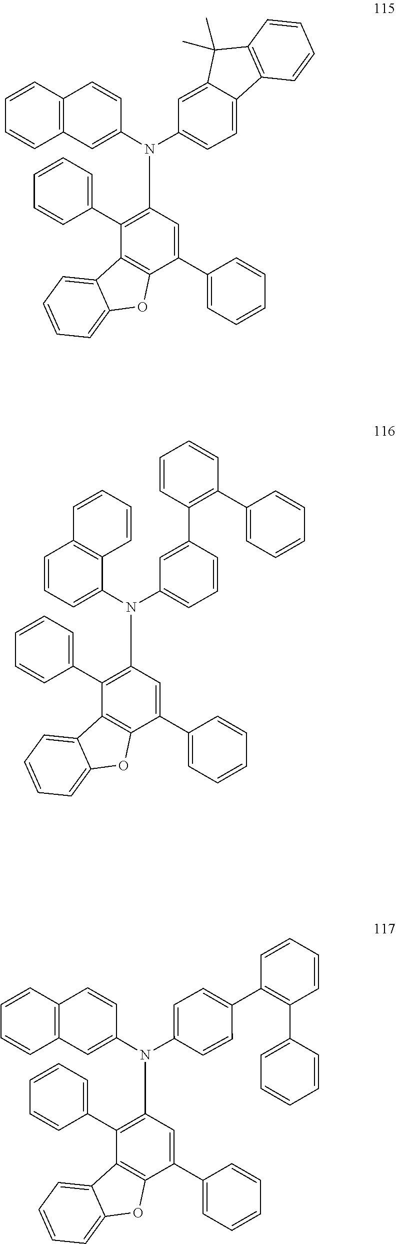 [SMILES: CC1(C)c2ccccc2-c2ccc(N(c3ccc4ccccc4c3)c3cc(-c4ccccc4)c4oc5ccccc5c4c3-c3ccccc3)cc21 ; c1ccc(-c2ccccc2-c2cccc(N(c3cc(-c4ccccc4)c4oc5ccccc5c4c3-c3ccccc3)c3cccc4ccccc34)c2)cc1 ; c1ccc(-c2ccccc2-c2ccc(N(c3ccc4ccccc4c3)c3cc(-c4ccccc4)c4oc5ccccc5c4c3-c3ccccc3)cc2)cc1]

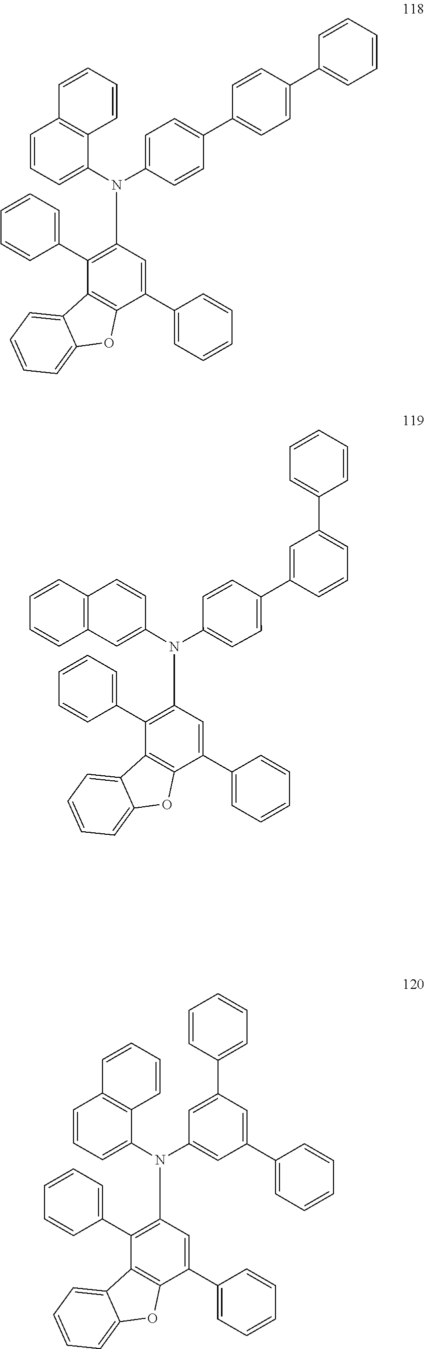 [SMILES: c1ccc(-c2ccc(-c3ccc(N(c4cc(-c5ccccc5)c5oc6ccccc6c5c4-c4ccccc4)c4cccc5ccccc45)cc3)cc2)cc1 ; c1ccc(-c2cccc(-c3ccc(N(c4ccc5ccccc5c4)c4cc(-c5ccccc5)c5oc6ccccc6c5c4-c4ccccc4)cc3)c2)cc1 ; c1ccc(-c2cc(-c3ccccc3)cc(N(c3cc(-c4ccccc4)c4oc5ccccc5c4c3-c3ccccc3)c3cccc4ccccc34)c2)cc1]

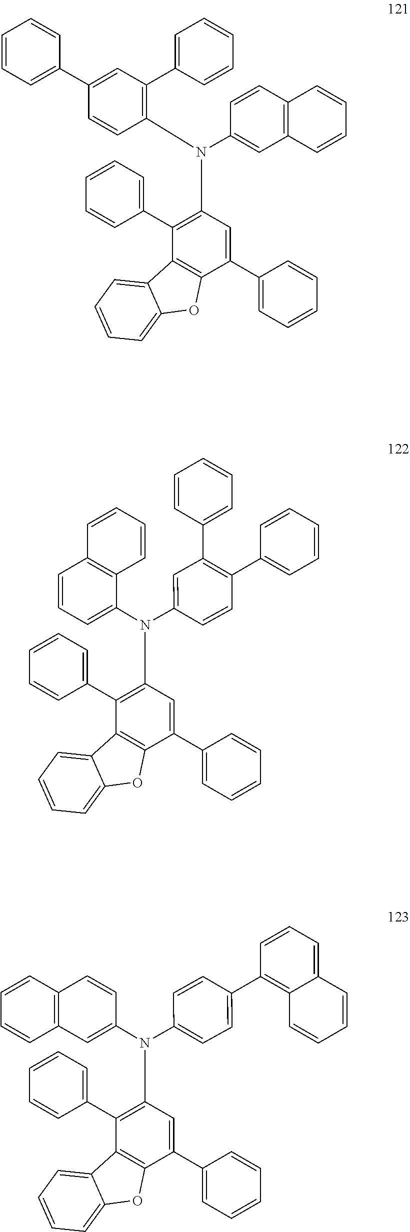 [SMILES: c1ccc(-c2ccc(N(c3ccc4ccccc4c3)c3cc(-c4ccccc4)c4oc5ccccc5c4c3-c3ccccc3)c(-c3ccccc3)c2)cc1 ; c1ccc(-c2ccc(N(c3cc(-c4ccccc4)c4oc5ccccc5c4c3-c3ccccc3)c3cccc4ccccc34)cc2-c2ccccc2)cc1 ; c1ccc(-c2cc(N(c3ccc(-c4cccc5ccccc45)cc3)c3ccc4ccccc4c3)c(-c3ccccc3)c3c2oc2ccccc23)cc1]

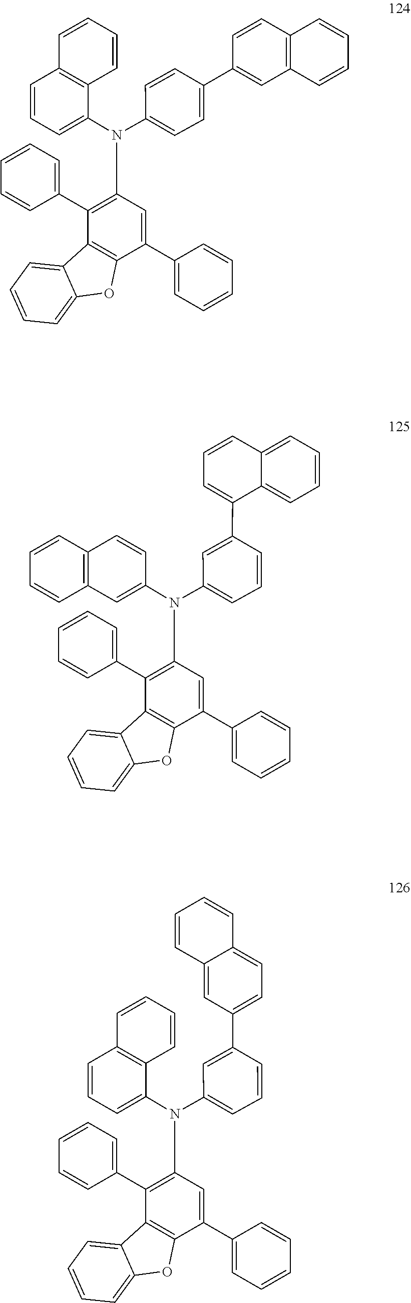 [SMILES: c1ccc(-c2cc(N(c3ccc(-c4ccc5ccccc5c4)cc3)c3cccc4ccccc34)c(-c3ccccc3)c3c2oc2ccccc23)cc1 ; c1ccc(-c2cc(N(c3cccc(-c4cccc5ccccc45)c3)c3ccc4ccccc4c3)c(-c3ccccc3)c3c2oc2ccccc23)cc1 ; c1ccc(-c2cc(N(c3cccc(-c4ccc5ccccc5c4)c3)c3cccc4ccccc34)c(-c3ccccc3)c3c2oc2ccccc23)cc1]

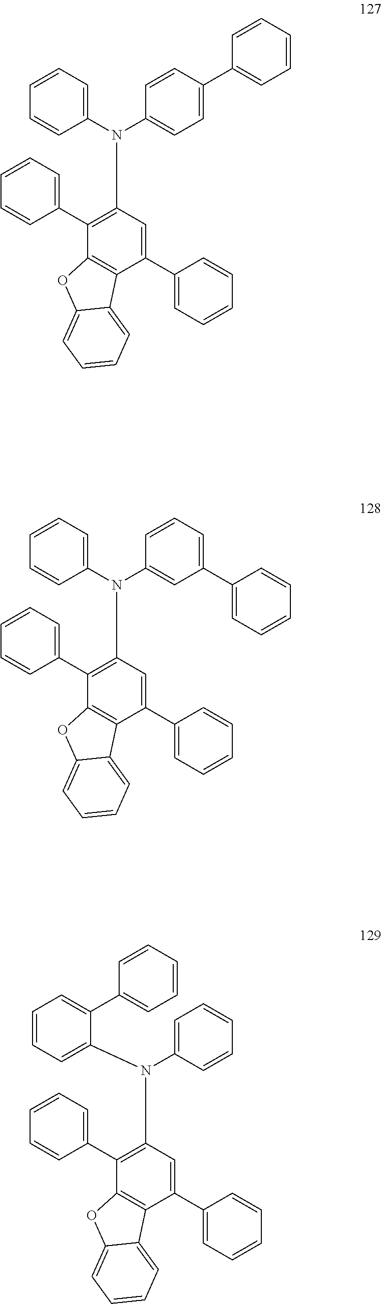 [SMILES: c1ccc(-c2ccc(N(c3ccccc3)c3cc(-c4ccccc4)c4c(oc5ccccc54)c3-c3ccccc3)cc2)cc1 ; c1ccc(-c2cccc(N(c3ccccc3)c3cc(-c4ccccc4)c4c(oc5ccccc54)c3-c3ccccc3)c2)cc1 ; c1ccc(-c2ccccc2N(c2ccccc2)c2cc(-c3ccccc3)c3c(oc4ccccc43)c2-c2ccccc2)cc1]

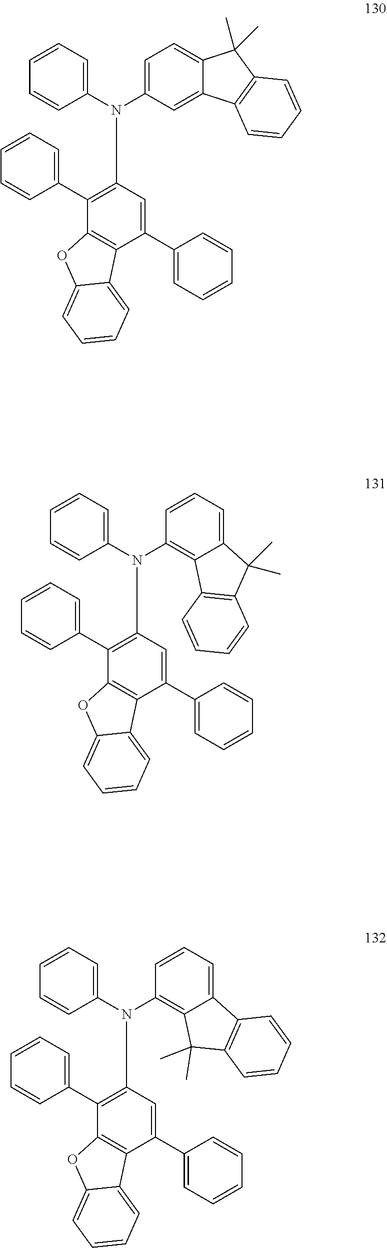 [SMILES: CC1(C)c2ccccc2-c2cc(N(c3ccccc3)c3cc(-c4ccccc4)c4c(oc5ccccc54)c3-c3ccccc3)ccc21 ; CC1(C)c2ccccc2-c2c(N(c3ccccc3)c3cc(-c4ccccc4)c4c(oc5ccccc54)c3-c3ccccc3)cccc21 ; CC1(C)c2ccccc2-c2cccc(N(c3ccccc3)c3cc(-c4ccccc4)c4c(oc5ccccc54)c3-c3ccccc3)c21]

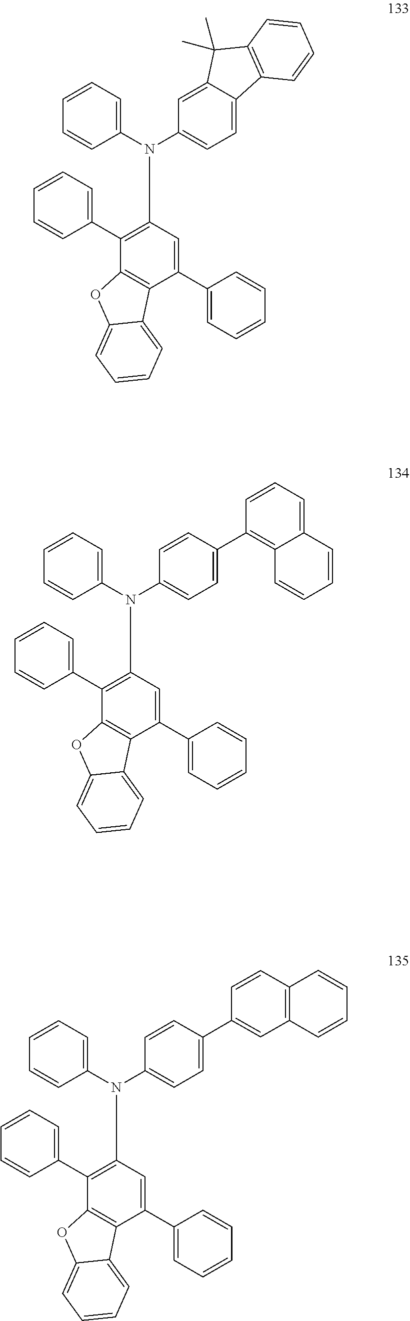 [SMILES: CC1(C)c2ccccc2-c2ccc(N(c3ccccc3)c3cc(-c4ccccc4)c4c(oc5ccccc54)c3-c3ccccc3)cc21 ; c1ccc(-c2c(N(c3ccccc3)c3ccc(-c4cccc5ccccc45)cc3)cc(-c3ccccc3)c3c2oc2ccccc23)cc1 ; c1ccc(-c2c(N(c3ccccc3)c3ccc(-c4ccc5ccccc5c4)cc3)cc(-c3ccccc3)c3c2oc2ccccc23)cc1]

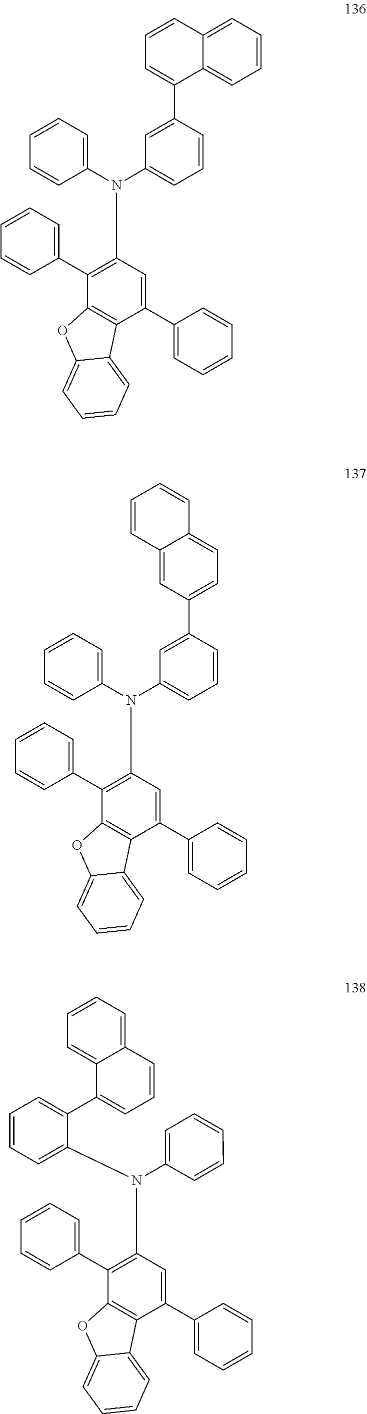 [SMILES: c1ccc(-c2c(N(c3ccccc3)c3cccc(-c4cccc5ccccc45)c3)cc(-c3ccccc3)c3c2oc2ccccc23)cc1 ; c1ccc(-c2c(N(c3ccccc3)c3cccc(-c4ccc5ccccc5c4)c3)cc(-c3ccccc3)c3c2oc2ccccc23)cc1 ; c1ccc(-c2c(N(c3ccccc3)c3ccccc3-c3cccc4ccccc34)cc(-c3ccccc3)c3c2oc2ccccc23)cc1]

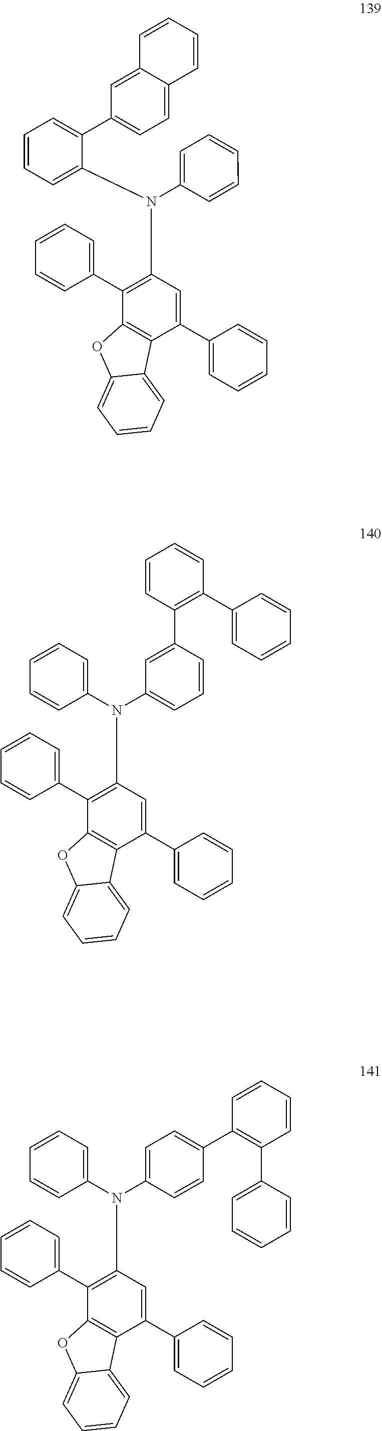 [SMILES: c1ccc(-c2c(N(c3ccccc3)c3ccccc3-c3ccc4ccccc4c3)cc(-c3ccccc3)c3c2oc2ccccc23)cc1 ; c1ccc(-c2ccccc2-c2cccc(N(c3ccccc3)c3cc(-c4ccccc4)c4c(oc5ccccc54)c3-c3ccccc3)c2)cc1 ; c1ccc(-c2ccccc2-c2ccc(N(c3ccccc3)c3cc(-c4ccccc4)c4c(oc5ccccc54)c3-c3ccccc3)cc2)cc1]

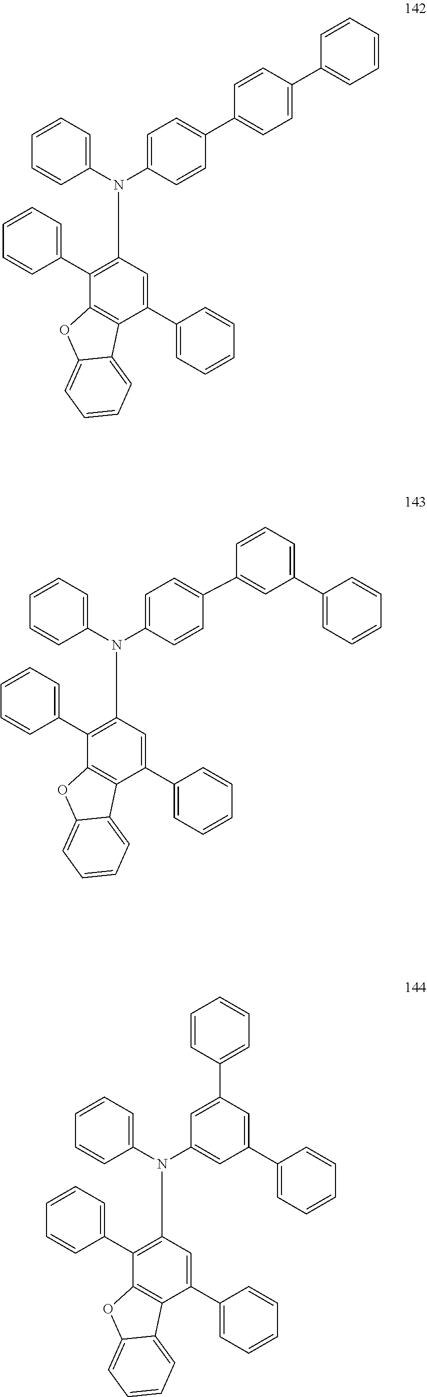 [SMILES: c1ccc(-c2ccc(-c3ccc(N(c4ccccc4)c4cc(-c5ccccc5)c5c(oc6ccccc65)c4-c4ccccc4)cc3)cc2)cc1 ; c1ccc(-c2cccc(-c3ccc(N(c4ccccc4)c4cc(-c5ccccc5)c5c(oc6ccccc65)c4-c4ccccc4)cc3)c2)cc1 ; c1ccc(-c2cc(-c3ccccc3)cc(N(c3ccccc3)c3cc(-c4ccccc4)c4c(oc5ccccc54)c3-c3ccccc3)c2)cc1]

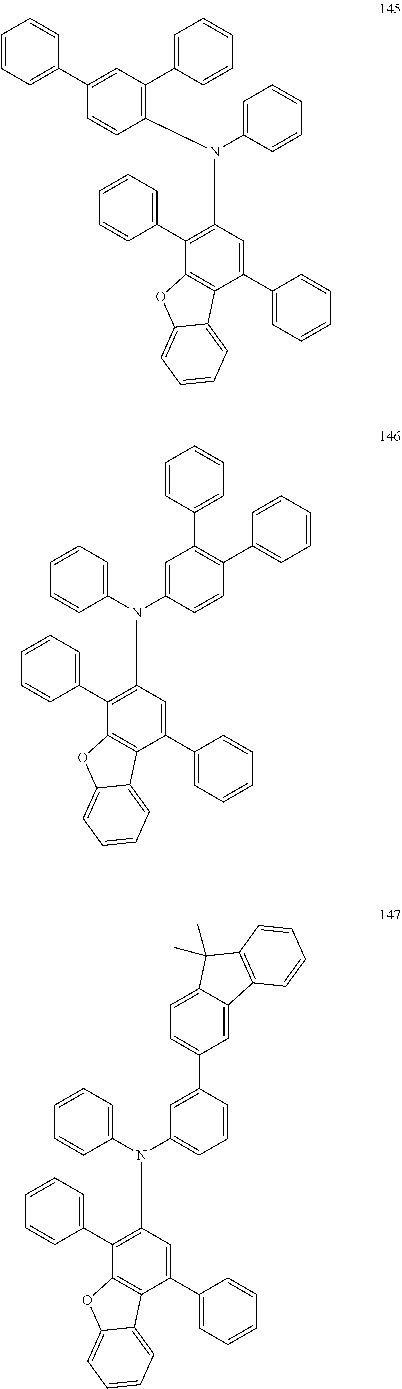 [SMILES: c1ccc(-c2ccc(N(c3ccccc3)c3cc(-c4ccccc4)c4c(oc5ccccc54)c3-c3ccccc3)c(-c3ccccc3)c2)cc1 ; c1ccc(-c2ccc(N(c3ccccc3)c3cc(-c4ccccc4)c4c(oc5ccccc54)c3-c3ccccc3)cc2-c2ccccc2)cc1 ; CC1(C)c2ccccc2-c2cc(-c3cccc(N(c4ccccc4)c4cc(-c5ccccc5)c5c(oc6ccccc65)c4-c4ccccc4)c3)ccc21]

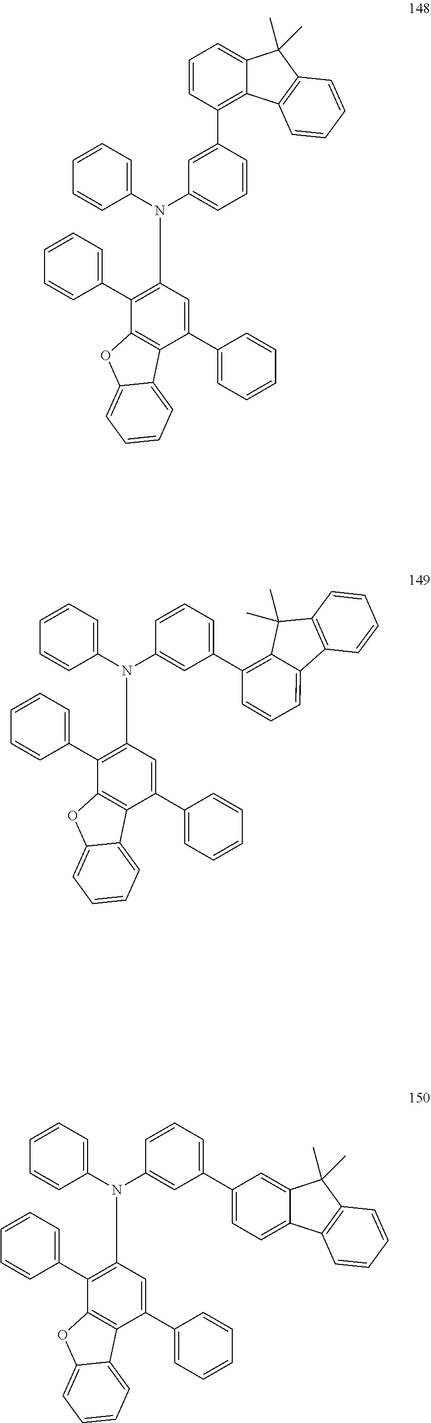 [SMILES: CC1(C)c2ccccc2-c2c(-c3cccc(N(c4ccccc4)c4cc(-c5ccccc5)c5c(oc6ccccc65)c4-c4ccccc4)c3)cccc21 ; CC1(C)c2ccccc2-c2cccc(-c3cccc(N(c4ccccc4)c4cc(-c5ccccc5)c5c(oc6ccccc65)c4-c4ccccc4)c3)c21 ; CC1(C)c2ccccc2-c2ccc(-c3cccc(N(c4ccccc4)c4cc(-c5ccccc5)c5c(oc6ccccc65)c4-c4ccccc4)c3)cc21]

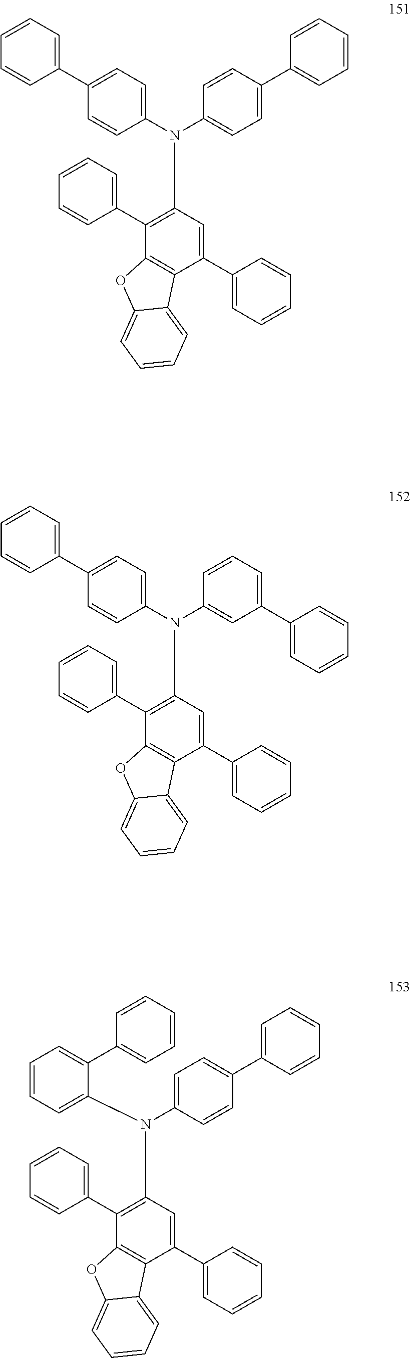 [SMILES: c1ccc(-c2ccc(N(c3ccc(-c4ccccc4)cc3)c3cc(-c4ccccc4)c4c(oc5ccccc54)c3-c3ccccc3)cc2)cc1 ; c1ccc(-c2ccc(N(c3cccc(-c4ccccc4)c3)c3cc(-c4ccccc4)c4c(oc5ccccc54)c3-c3ccccc3)cc2)cc1 ; c1ccc(-c2ccc(N(c3ccccc3-c3ccccc3)c3cc(-c4ccccc4)c4c(oc5ccccc54)c3-c3ccccc3)cc2)cc1]

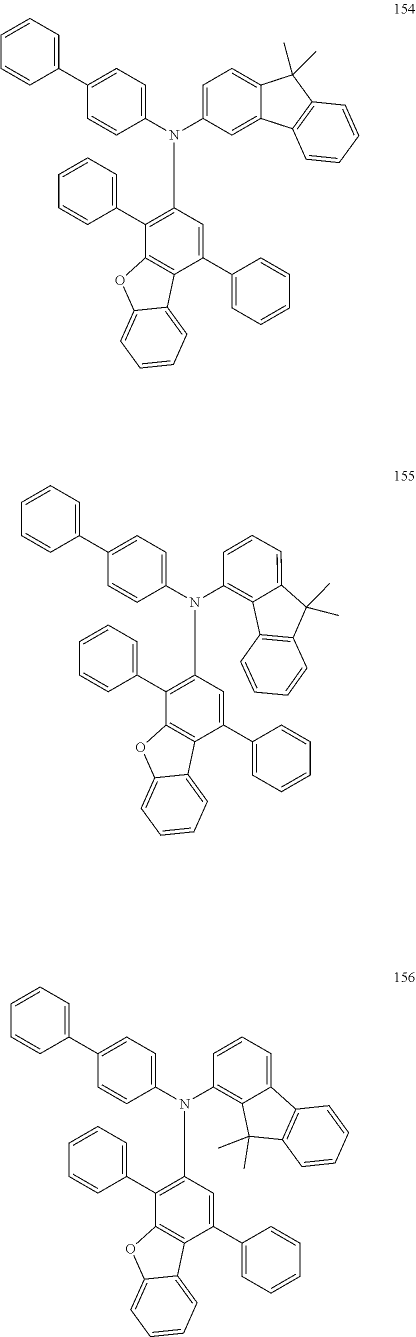 [SMILES: CC1(C)c2ccccc2-c2cc(N(c3ccc(-c4ccccc4)cc3)c3cc(-c4ccccc4)c4c(oc5ccccc54)c3-c3ccccc3)ccc21 ; CC1(C)c2ccccc2-c2c(N(c3ccc(-c4ccccc4)cc3)c3cc(-c4ccccc4)c4c(oc5ccccc54)c3-c3ccccc3)cccc21 ; CC1(C)c2ccccc2-c2cccc(N(c3ccc(-c4ccccc4)cc3)c3cc(-c4ccccc4)c4c(oc5ccccc54)c3-c3ccccc3)c21]

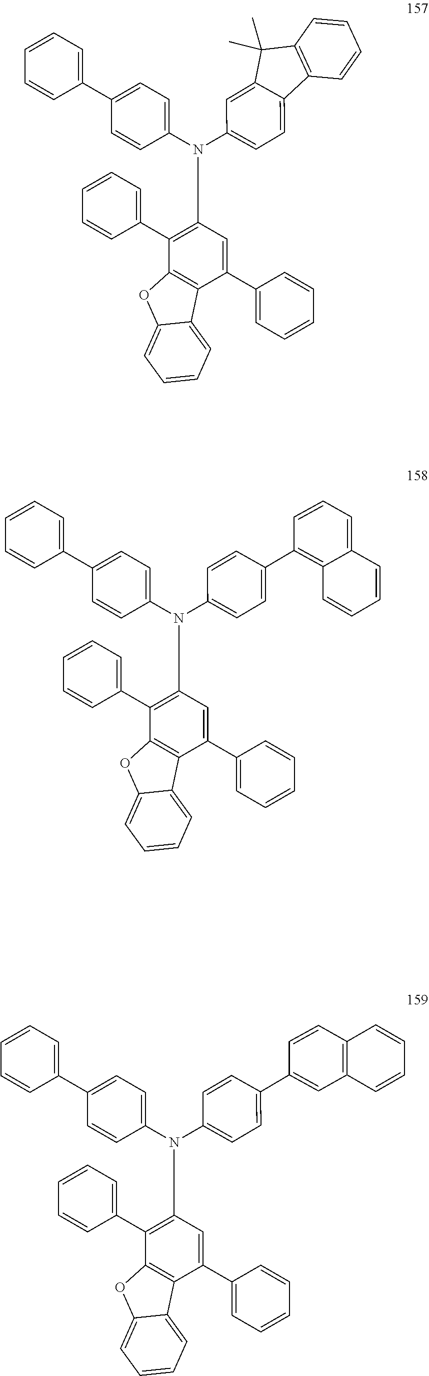 [SMILES: CC1(C)c2ccccc2-c2ccc(N(c3ccc(-c4ccccc4)cc3)c3cc(-c4ccccc4)c4c(oc5ccccc54)c3-c3ccccc3)cc21 ; c1ccc(-c2ccc(N(c3ccc(-c4cccc5ccccc45)cc3)c3cc(-c4ccccc4)c4c(oc5ccccc54)c3-c3ccccc3)cc2)cc1 ; c1ccc(-c2ccc(N(c3ccc(-c4ccc5ccccc5c4)cc3)c3cc(-c4ccccc4)c4c(oc5ccccc54)c3-c3ccccc3)cc2)cc1]

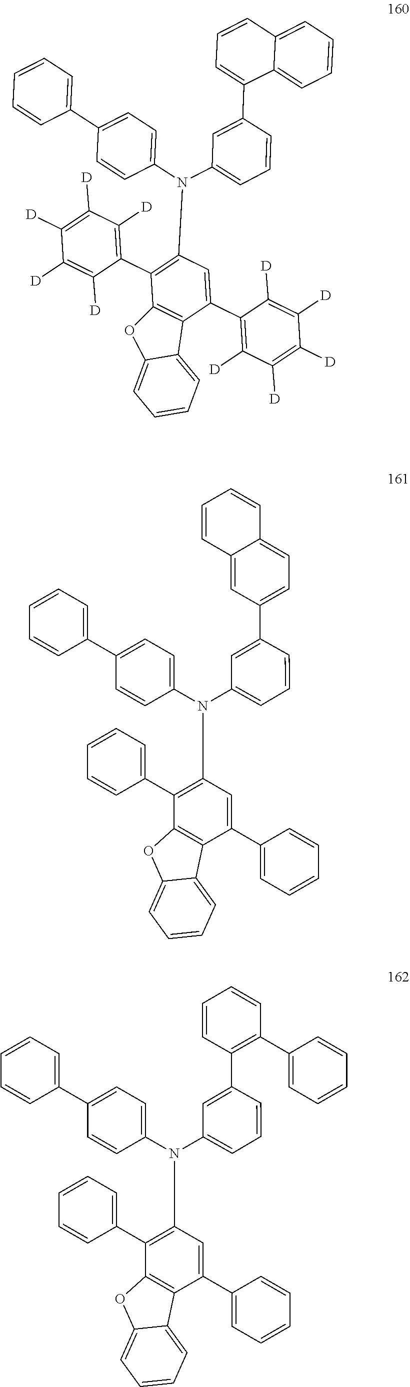 [SMILES: [2H]c1c([2H])c([2H])c(-c2c(N(c3ccc(-c4ccccc4)cc3)c3cccc(-c4cccc5ccccc45)c3)cc(-c3c([2H])c([2H])c([2H])c([2H])c3[2H])c3c2oc2ccccc23)c([2H])c1[2H] ; c1ccc(-c2ccc(N(c3cccc(-c4ccc5ccccc5c4)c3)c3cc(-c4ccccc4)c4c(oc5ccccc54)c3-c3ccccc3)cc2)cc1 ; c1ccc(-c2ccc(N(c3cccc(-c4ccccc4-c4ccccc4)c3)c3cc(-c4ccccc4)c4c(oc5ccccc54)c3-c3ccccc3)cc2)cc1]

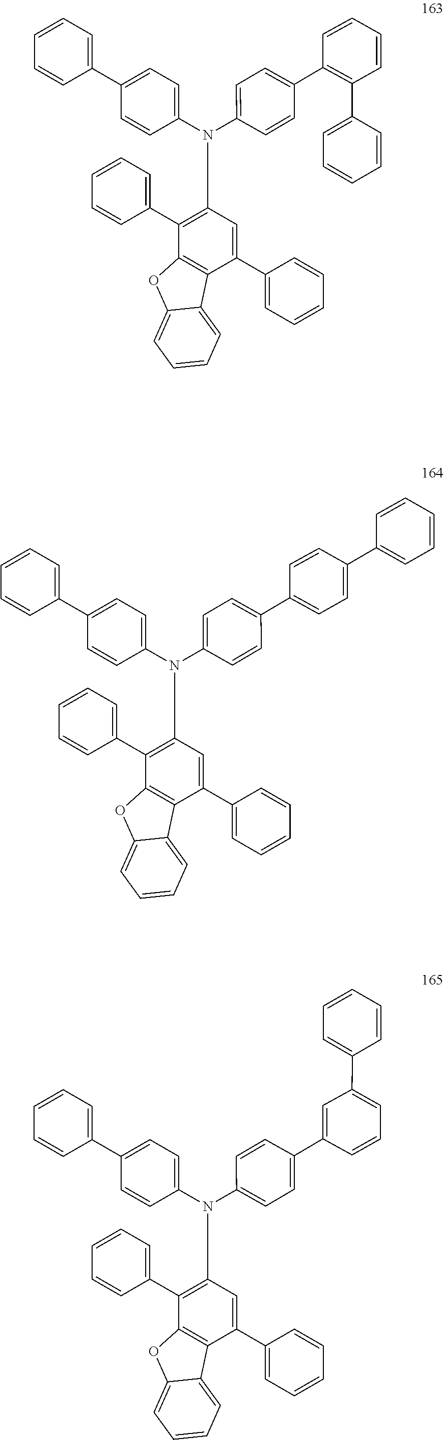 [SMILES: c1ccc(-c2ccc(N(c3ccc(-c4ccccc4-c4ccccc4)cc3)c3cc(-c4ccccc4)c4c(oc5ccccc54)c3-c3ccccc3)cc2)cc1 ; c1ccc(-c2ccc(-c3ccc(N(c4ccc(-c5ccccc5)cc4)c4cc(-c5ccccc5)c5c(oc6ccccc65)c4-c4ccccc4)cc3)cc2)cc1 ; c1ccc(-c2ccc(N(c3ccc(-c4cccc(-c5ccccc5)c4)cc3)c3cc(-c4ccccc4)c4c(oc5ccccc54)c3-c3ccccc3)cc2)cc1]

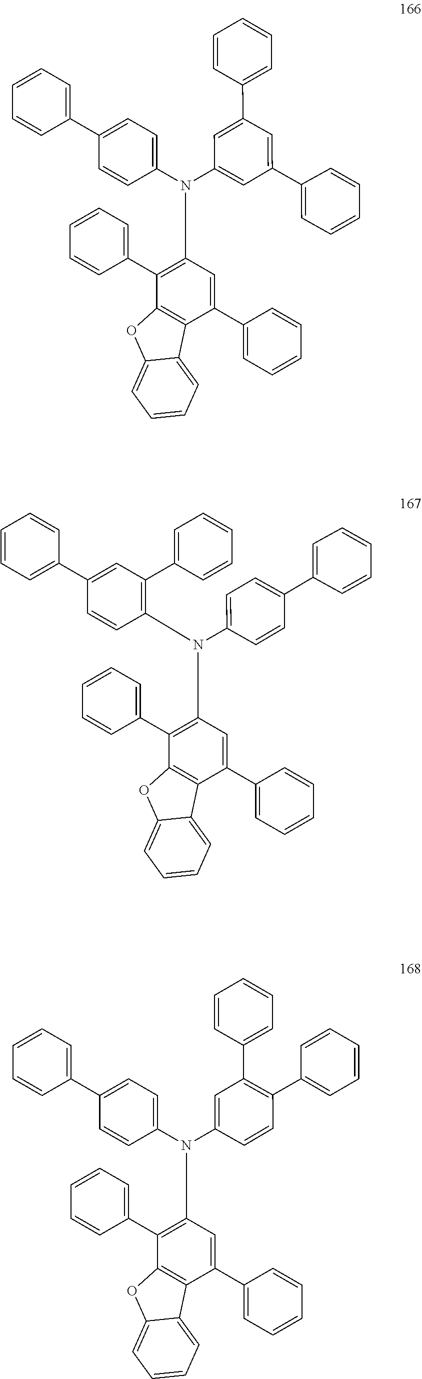 [SMILES: c1ccc(-c2ccc(N(c3cc(-c4ccccc4)cc(-c4ccccc4)c3)c3cc(-c4ccccc4)c4c(oc5ccccc54)c3-c3ccccc3)cc2)cc1 ; c1ccc(-c2ccc(N(c3ccc(-c4ccccc4)cc3-c3ccccc3)c3cc(-c4ccccc4)c4c(oc5ccccc54)c3-c3ccccc3)cc2)cc1 ; c1ccc(-c2ccc(N(c3ccc(-c4ccccc4)c(-c4ccccc4)c3)c3cc(-c4ccccc4)c4c(oc5ccccc54)c3-c3ccccc3)cc2)cc1]

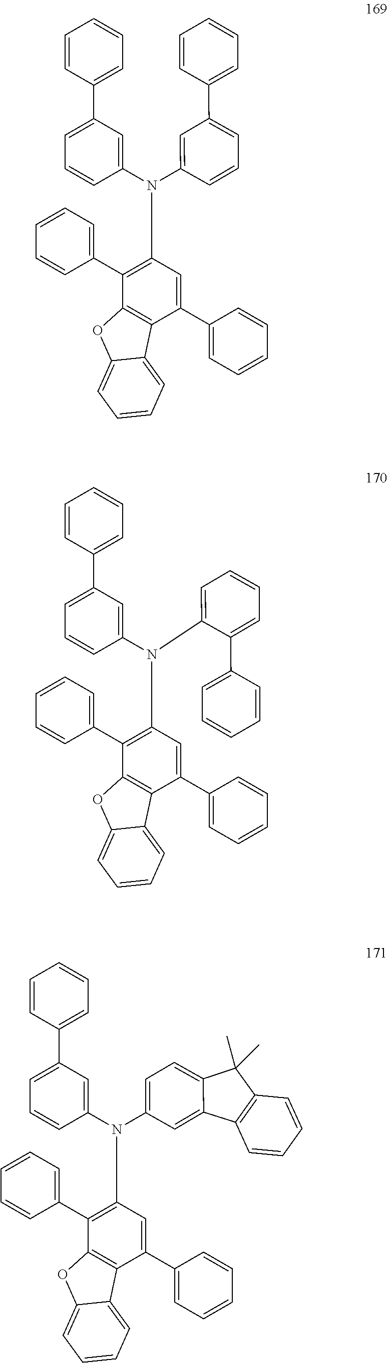 [SMILES: c1ccc(-c2cccc(N(c3cccc(-c4ccccc4)c3)c3cc(-c4ccccc4)c4c(oc5ccccc54)c3-c3ccccc3)c2)cc1 ; c1ccc(-c2cccc(N(c3ccccc3-c3ccccc3)c3cc(-c4ccccc4)c4c(oc5ccccc54)c3-c3ccccc3)c2)cc1 ; CC1(C)c2ccccc2-c2cc(N(c3cccc(-c4ccccc4)c3)c3cc(-c4ccccc4)c4c(oc5ccccc54)c3-c3ccccc3)ccc21]

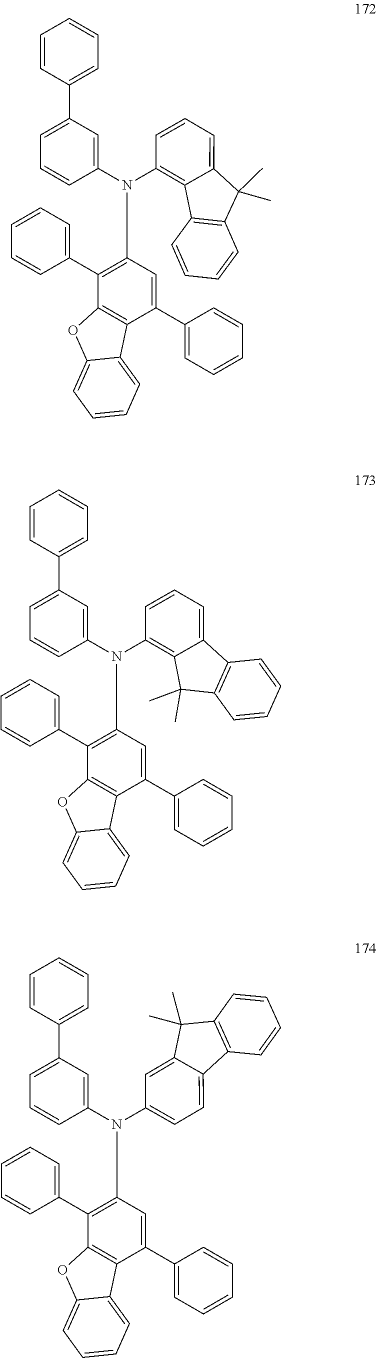 [SMILES: CC1(C)c2ccccc2-c2c(N(c3cccc(-c4ccccc4)c3)c3cc(-c4ccccc4)c4c(oc5ccccc54)c3-c3ccccc3)cccc21 ; CC1(C)c2ccccc2-c2cccc(N(c3cccc(-c4ccccc4)c3)c3cc(-c4ccccc4)c4c(oc5ccccc54)c3-c3ccccc3)c21 ; CC1(C)c2ccccc2-c2ccc(N(c3cccc(-c4ccccc4)c3)c3cc(-c4ccccc4)c4c(oc5ccccc54)c3-c3ccccc3)cc21]

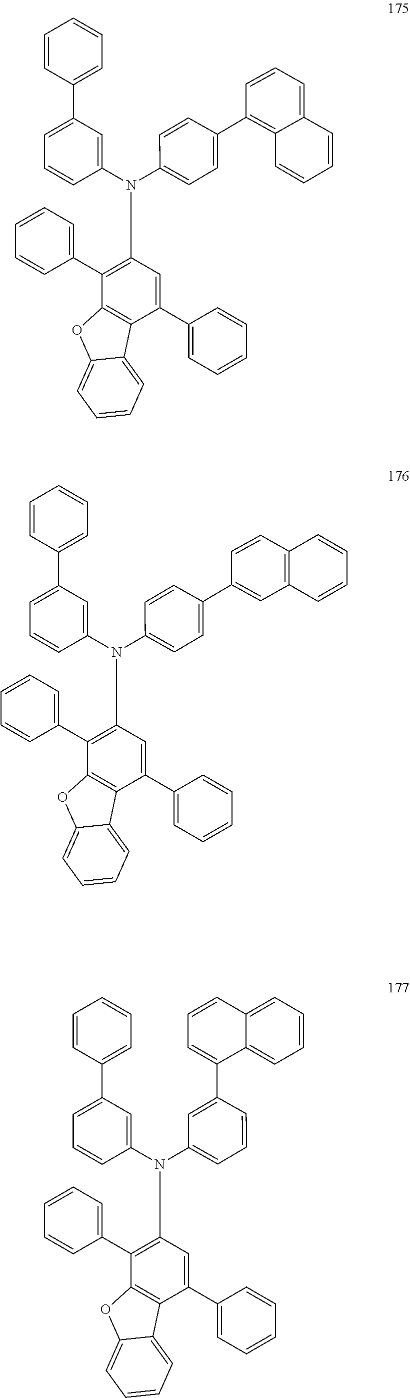 [SMILES: c1ccc(-c2cccc(N(c3ccc(-c4cccc5ccccc45)cc3)c3cc(-c4ccccc4)c4c(oc5ccccc54)c3-c3ccccc3)c2)cc1 ; c1ccc(-c2cccc(N(c3ccc(-c4ccc5ccccc5c4)cc3)c3cc(-c4ccccc4)c4c(oc5ccccc54)c3-c3ccccc3)c2)cc1 ; c1ccc(-c2cccc(N(c3cccc(-c4cccc5ccccc45)c3)c3cc(-c4ccccc4)c4c(oc5ccccc54)c3-c3ccccc3)c2)cc1]

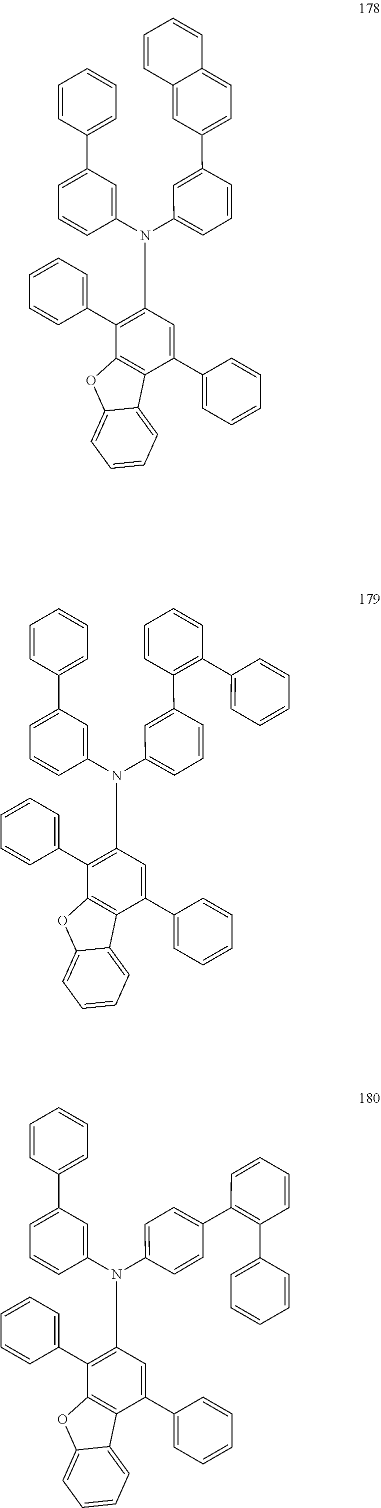 [SMILES: c1ccc(-c2cccc(N(c3cccc(-c4ccc5ccccc5c4)c3)c3cc(-c4ccccc4)c4c(oc5ccccc54)c3-c3ccccc3)c2)cc1 ; c1ccc(-c2cccc(N(c3cccc(-c4ccccc4-c4ccccc4)c3)c3cc(-c4ccccc4)c4c(oc5ccccc54)c3-c3ccccc3)c2)cc1 ; c1ccc(-c2cccc(N(c3ccc(-c4ccccc4-c4ccccc4)cc3)c3cc(-c4ccccc4)c4c(oc5ccccc54)c3-c3ccccc3)c2)cc1]

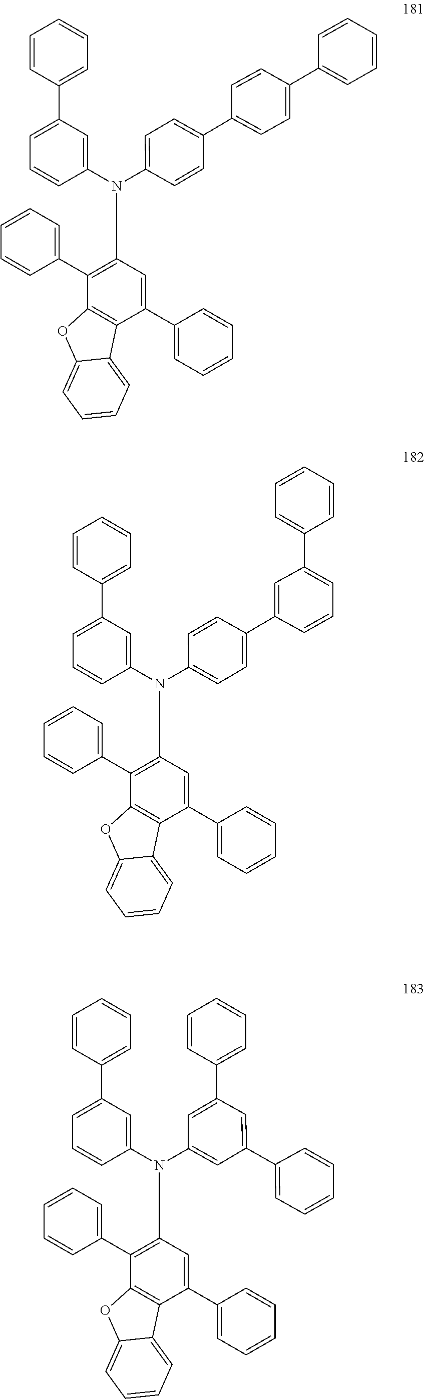 [SMILES: c1ccc(-c2ccc(-c3ccc(N(c4cccc(-c5ccccc5)c4)c4cc(-c5ccccc5)c5c(oc6ccccc65)c4-c4ccccc4)cc3)cc2)cc1 ; c1ccc(-c2cccc(-c3ccc(N(c4cccc(-c5ccccc5)c4)c4cc(-c5ccccc5)c5c(oc6ccccc65)c4-c4ccccc4)cc3)c2)cc1 ; c1ccc(-c2cccc(N(c3cc(-c4ccccc4)cc(-c4ccccc4)c3)c3cc(-c4ccccc4)c4c(oc5ccccc54)c3-c3ccccc3)c2)cc1]

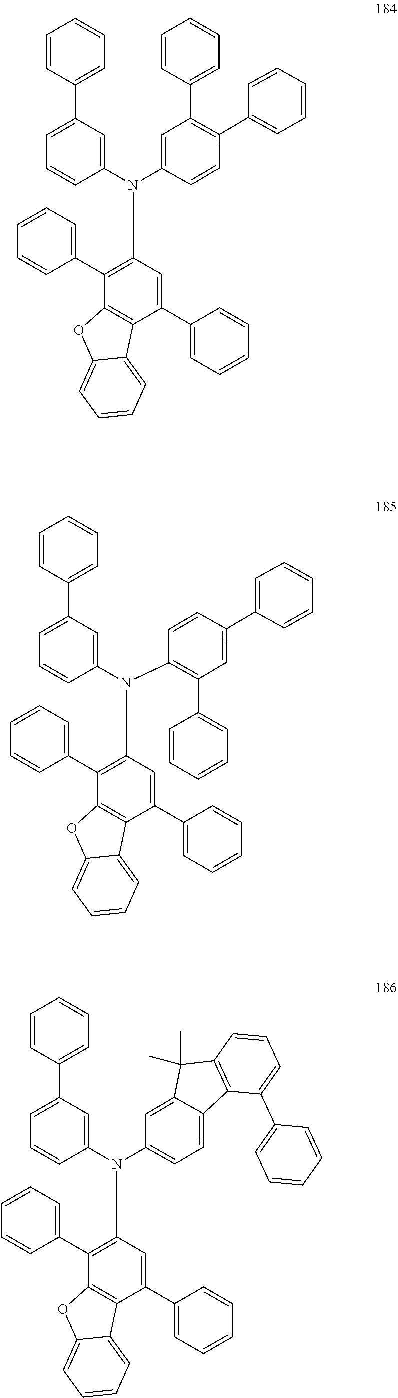 [SMILES: c1ccc(-c2cccc(N(c3ccc(-c4ccccc4)c(-c4ccccc4)c3)c3cc(-c4ccccc4)c4c(oc5ccccc54)c3-c3ccccc3)c2)cc1 ; c1ccc(-c2cccc(N(c3ccc(-c4ccccc4)cc3-c3ccccc3)c3cc(-c4ccccc4)c4c(oc5ccccc54)c3-c3ccccc3)c2)cc1 ; CC1(C)c2cc(N(c3cccc(-c4ccccc4)c3)c3cc(-c4ccccc4)c4c(oc5ccccc54)c3-c3ccccc3)ccc2-c2c(-c3ccccc3)cccc21]

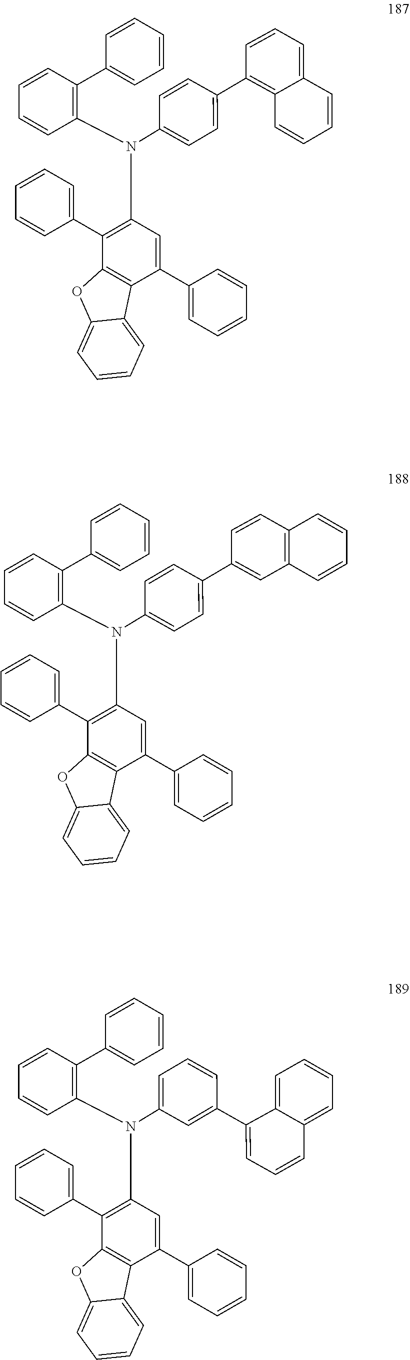 [SMILES: c1ccc(-c2ccccc2N(c2ccc(-c3cccc4ccccc34)cc2)c2cc(-c3ccccc3)c3c(oc4ccccc43)c2-c2ccccc2)cc1 ; c1ccc(-c2ccccc2N(c2ccc(-c3ccc4ccccc4c3)cc2)c2cc(-c3ccccc3)c3c(oc4ccccc43)c2-c2ccccc2)cc1 ; c1ccc(-c2ccccc2N(c2cccc(-c3cccc4ccccc34)c2)c2cc(-c3ccccc3)c3c(oc4ccccc43)c2-c2ccccc2)cc1]

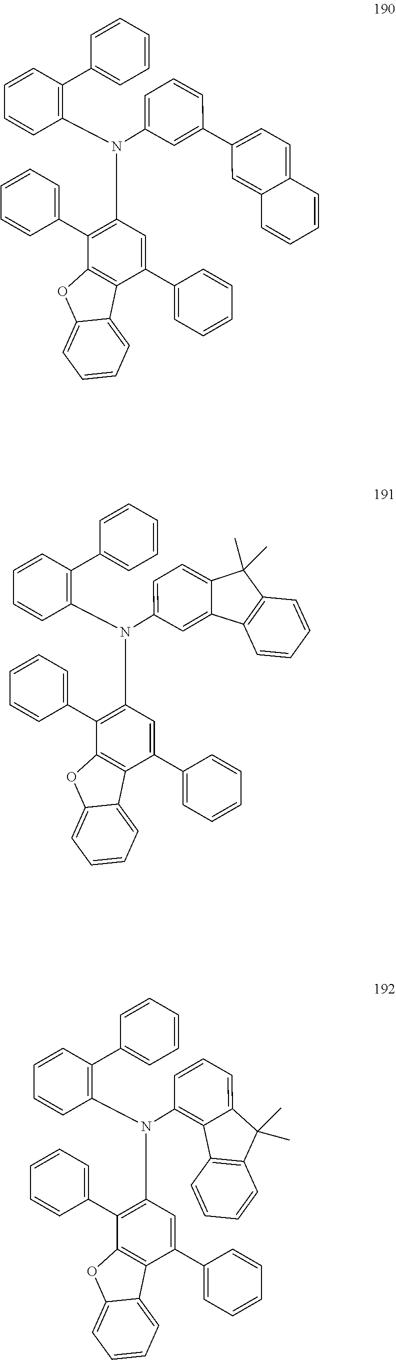 [SMILES: c1ccc(-c2ccccc2N(c2cccc(-c3ccc4ccccc4c3)c2)c2cc(-c3ccccc3)c3c(oc4ccccc43)c2-c2ccccc2)cc1 ; CC1(C)c2ccccc2-c2cc(N(c3ccccc3-c3ccccc3)c3cc(-c4ccccc4)c4c(oc5ccccc54)c3-c3ccccc3)ccc21 ; CC1(C)c2ccccc2-c2c(N(c3ccccc3-c3ccccc3)c3cc(-c4ccccc4)c4c(oc5ccccc54)c3-c3ccccc3)cccc21]

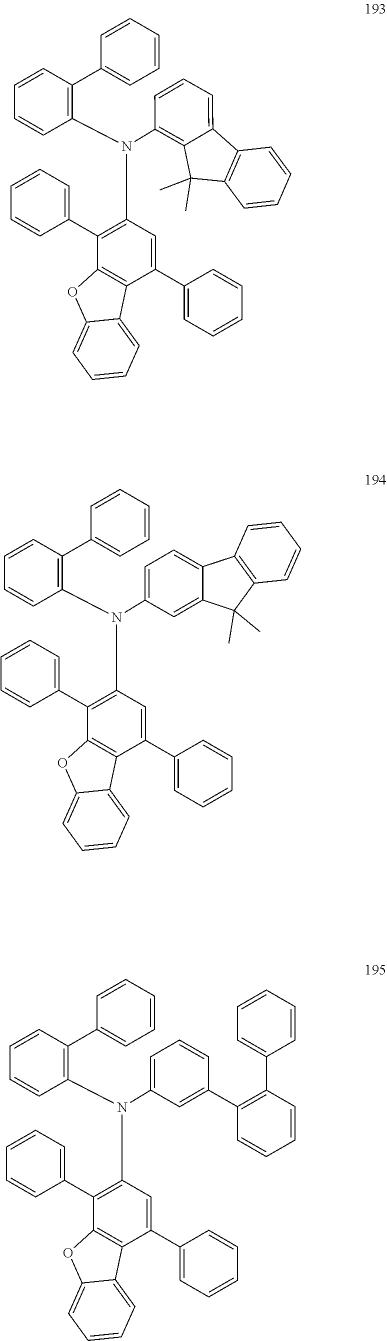 [SMILES: CC1(C)c2ccccc2-c2cccc(N(c3ccccc3-c3ccccc3)c3cc(-c4ccccc4)c4c(oc5ccccc54)c3-c3ccccc3)c21 ; CC1(C)c2ccccc2-c2ccc(N(c3ccccc3-c3ccccc3)c3cc(-c4ccccc4)c4c(oc5ccccc54)c3-c3ccccc3)cc21 ; c1ccc(-c2ccccc2-c2cccc(N(c3ccccc3-c3ccccc3)c3cc(-c4ccccc4)c4c(oc5ccccc54)c3-c3ccccc3)c2)cc1]

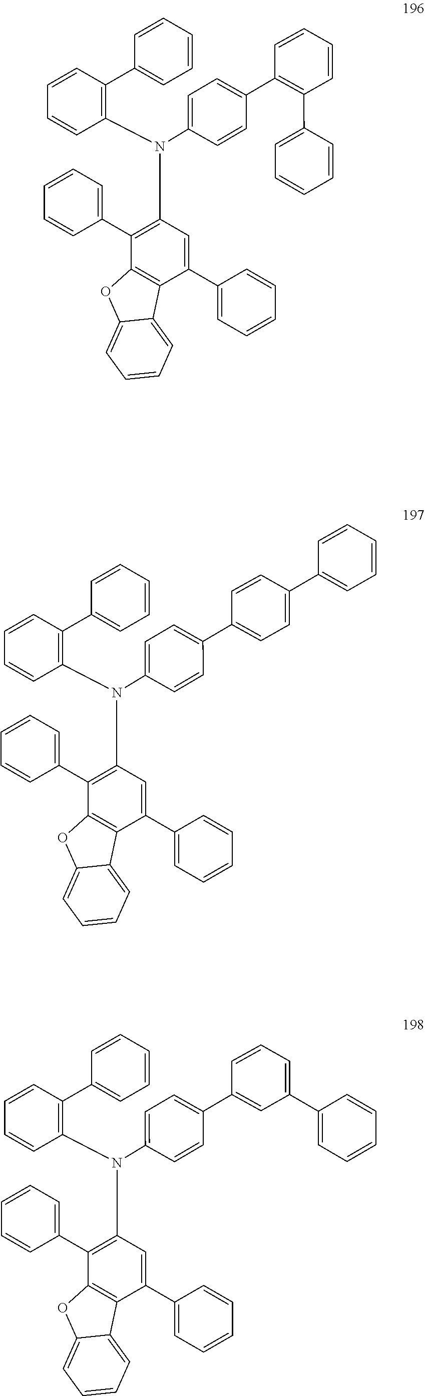 [SMILES: c1ccc(-c2ccccc2-c2ccc(N(c3ccccc3-c3ccccc3)c3cc(-c4ccccc4)c4c(oc5ccccc54)c3-c3ccccc3)cc2)cc1 ; c1ccc(-c2ccc(-c3ccc(N(c4ccccc4-c4ccccc4)c4cc(-c5ccccc5)c5c(oc6ccccc65)c4-c4ccccc4)cc3)cc2)cc1 ; c1ccc(-c2cccc(-c3ccc(N(c4ccccc4-c4ccccc4)c4cc(-c5ccccc5)c5c(oc6ccccc65)c4-c4ccccc4)cc3)c2)cc1]

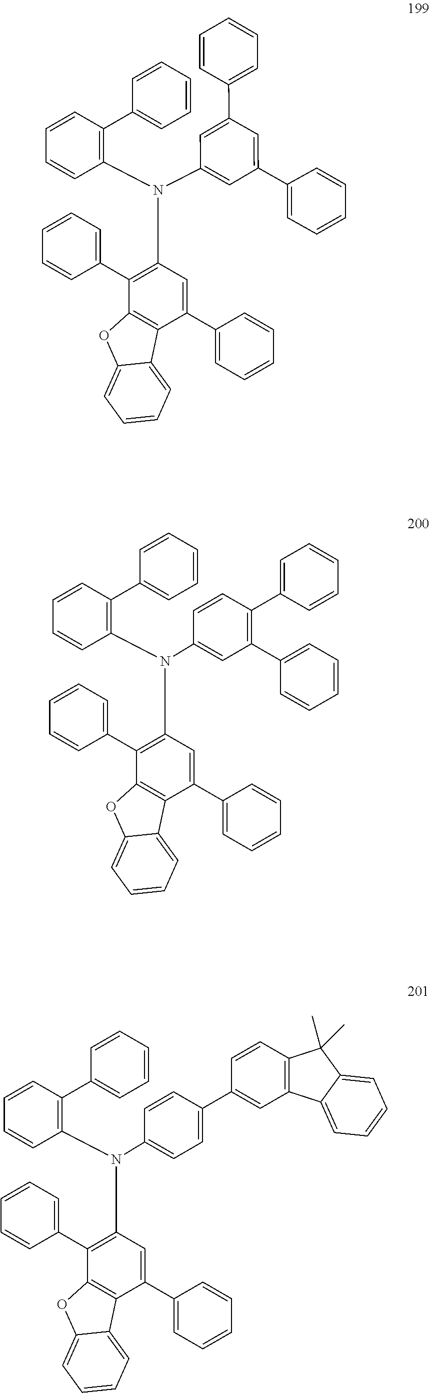 [SMILES: c1ccc(-c2cc(-c3ccccc3)cc(N(c3ccccc3-c3ccccc3)c3cc(-c4ccccc4)c4c(oc5ccccc54)c3-c3ccccc3)c2)cc1 ; c1ccc(-c2ccc(N(c3ccccc3-c3ccccc3)c3cc(-c4ccccc4)c4c(oc5ccccc54)c3-c3ccccc3)cc2-c2ccccc2)cc1 ; CC1(C)c2ccccc2-c2cc(-c3ccc(N(c4ccccc4-c4ccccc4)c4cc(-c5ccccc5)c5c(oc6ccccc65)c4-c4ccccc4)cc3)ccc21]

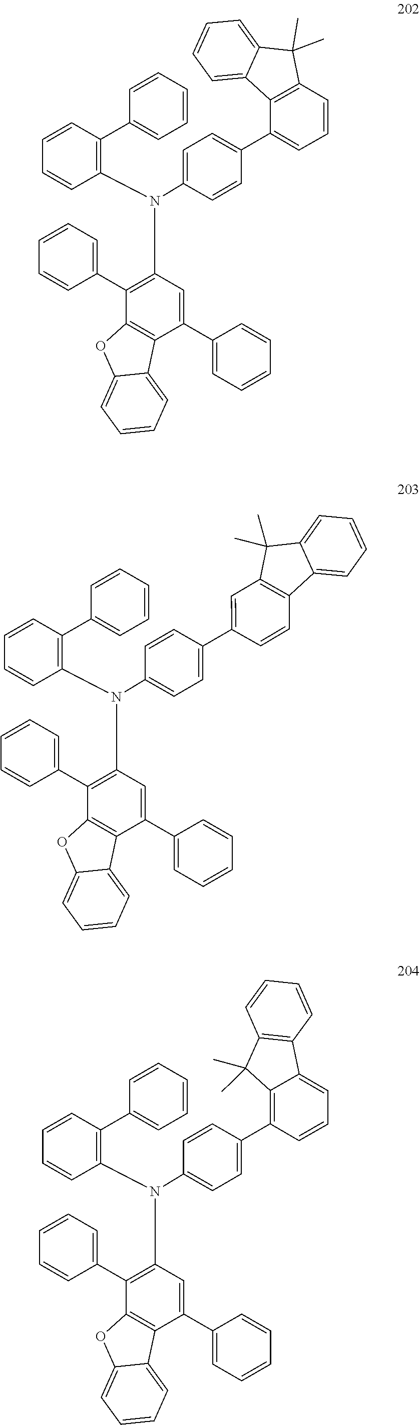 [SMILES: CC1(C)c2ccccc2-c2c(-c3ccc(N(c4ccccc4-c4ccccc4)c4cc(-c5ccccc5)c5c(oc6ccccc65)c4-c4ccccc4)cc3)cccc21 ; CC1(C)c2ccccc2-c2ccc(-c3ccc(N(c4ccccc4-c4ccccc4)c4cc(-c5ccccc5)c5c(oc6ccccc65)c4-c4ccccc4)cc3)cc21 ; CC1(C)c2ccccc2-c2cccc(-c3ccc(N(c4ccccc4-c4ccccc4)c4cc(-c5ccccc5)c5c(oc6ccccc65)c4-c4ccccc4)cc3)c21]

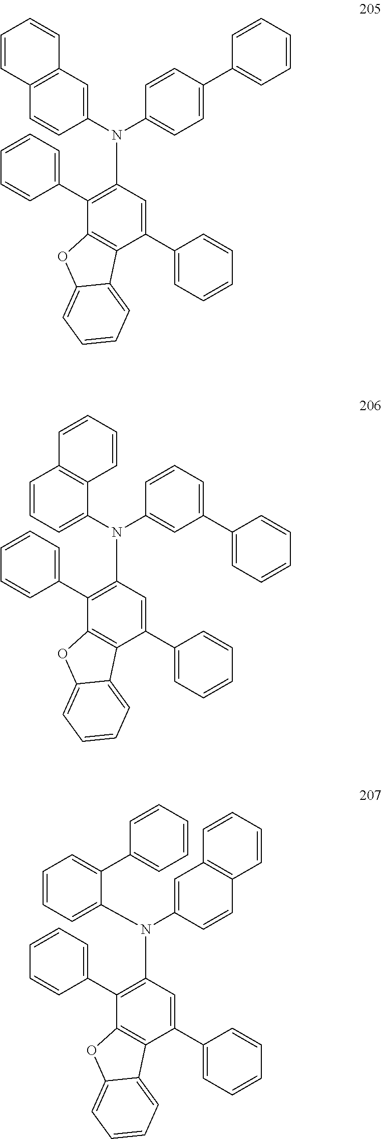 [SMILES: c1ccc(-c2ccc(N(c3ccc4ccccc4c3)c3cc(-c4ccccc4)c4c(oc5ccccc54)c3-c3ccccc3)cc2)cc1 ; c1ccc(-c2cccc(N(c3cc(-c4ccccc4)c4c(oc5ccccc54)c3-c3ccccc3)c3cccc4ccccc34)c2)cc1 ; c1ccc(-c2ccccc2N(c2ccc3ccccc3c2)c2cc(-c3ccccc3)c3c(oc4ccccc43)c2-c2ccccc2)cc1]

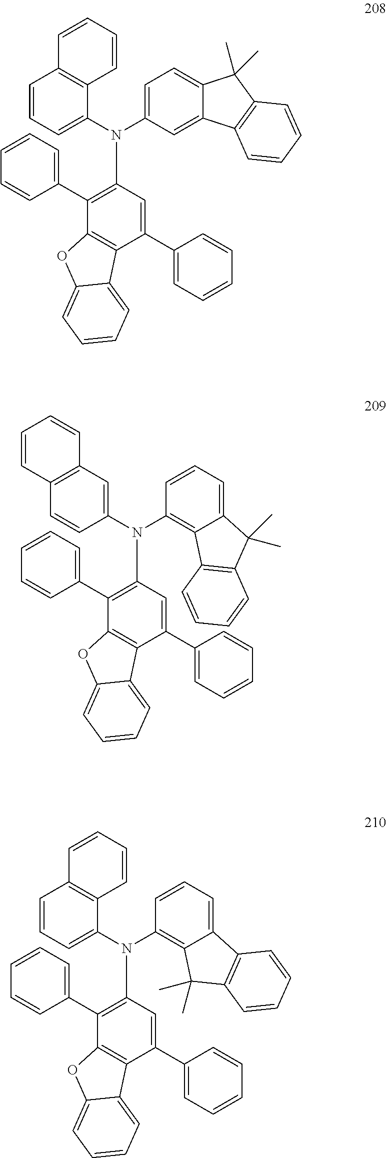 [SMILES: CC1(C)c2ccccc2-c2cc(N(c3cc(-c4ccccc4)c4c(oc5ccccc54)c3-c3ccccc3)c3cccc4ccccc34)ccc21 ; CC1(C)c2ccccc2-c2c(N(c3ccc4ccccc4c3)c3cc(-c4ccccc4)c4c(oc5ccccc54)c3-c3ccccc3)cccc21 ; CC1(C)c2ccccc2-c2cccc(N(c3cc(-c4ccccc4)c4c(oc5ccccc54)c3-c3ccccc3)c3cccc4ccccc34)c21]

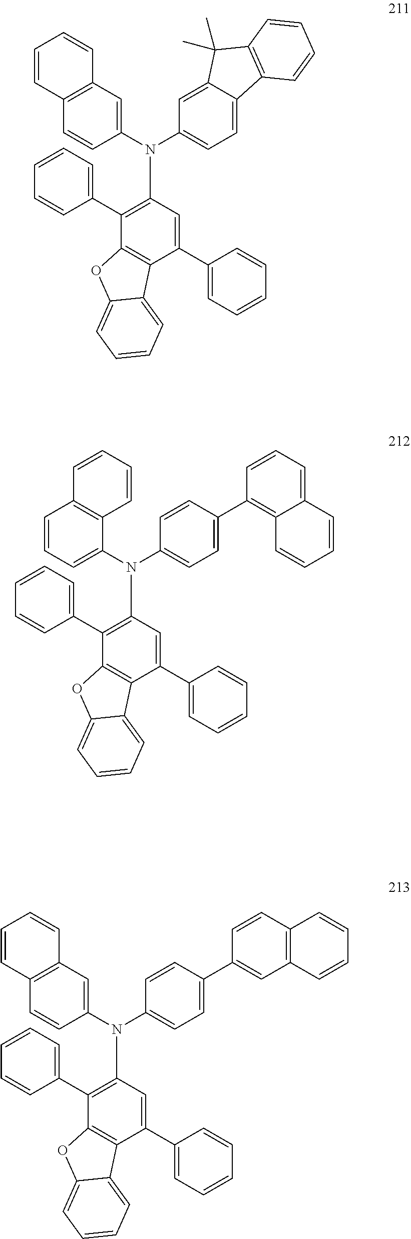 [SMILES: CC1(C)c2ccccc2-c2ccc(N(c3ccc4ccccc4c3)c3cc(-c4ccccc4)c4c(oc5ccccc54)c3-c3ccccc3)cc21 ; c1ccc(-c2c(N(c3ccc(-c4cccc5ccccc45)cc3)c3cccc4ccccc34)cc(-c3ccccc3)c3c2oc2ccccc23)cc1 ; c1ccc(-c2c(N(c3ccc(-c4ccc5ccccc5c4)cc3)c3ccc4ccccc4c3)cc(-c3ccccc3)c3c2oc2ccccc23)cc1]

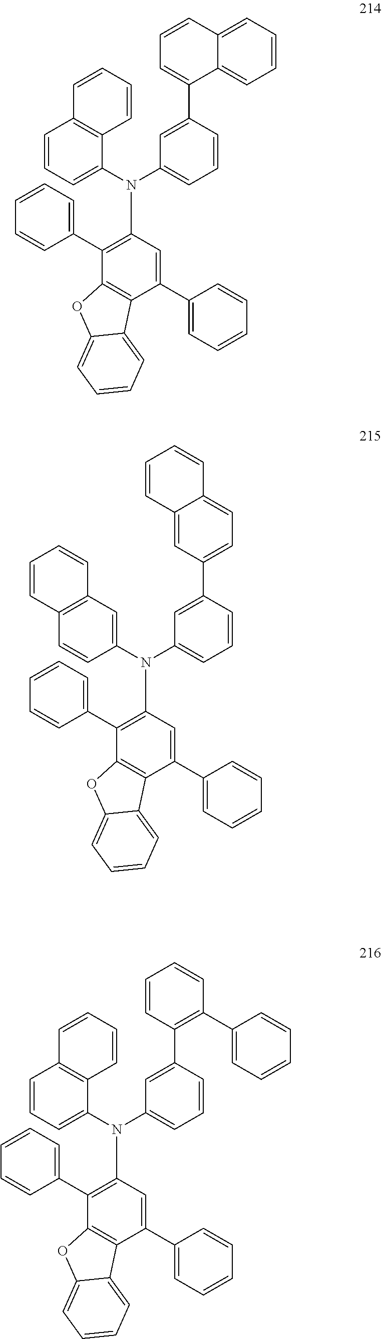 [SMILES: c1ccc(-c2c(N(c3cccc(-c4cccc5ccccc45)c3)c3cccc4ccccc34)cc(-c3ccccc3)c3c2oc2ccccc23)cc1 ; c1ccc(-c2c(N(c3cccc(-c4ccc5ccccc5c4)c3)c3ccc4ccccc4c3)cc(-c3ccccc3)c3c2oc2ccccc23)cc1 ; c1ccc(-c2ccccc2-c2cccc(N(c3cc(-c4ccccc4)c4c(oc5ccccc54)c3-c3ccccc3)c3cccc4ccccc34)c2)cc1]

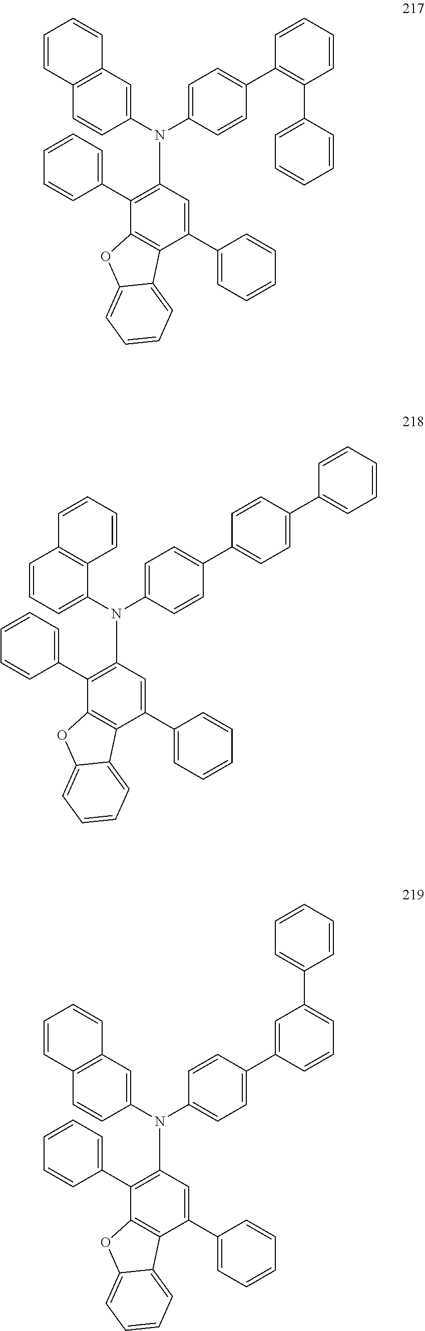 [SMILES: c1ccc(-c2ccccc2-c2ccc(N(c3ccc4ccccc4c3)c3cc(-c4ccccc4)c4c(oc5ccccc54)c3-c3ccccc3)cc2)cc1 ; c1ccc(-c2ccc(-c3ccc(N(c4cc(-c5ccccc5)c5c(oc6ccccc65)c4-c4ccccc4)c4cccc5ccccc45)cc3)cc2)cc1 ; c1ccc(-c2cccc(-c3ccc(N(c4ccc5ccccc5c4)c4cc(-c5ccccc5)c5c(oc6ccccc65)c4-c4ccccc4)cc3)c2)cc1]

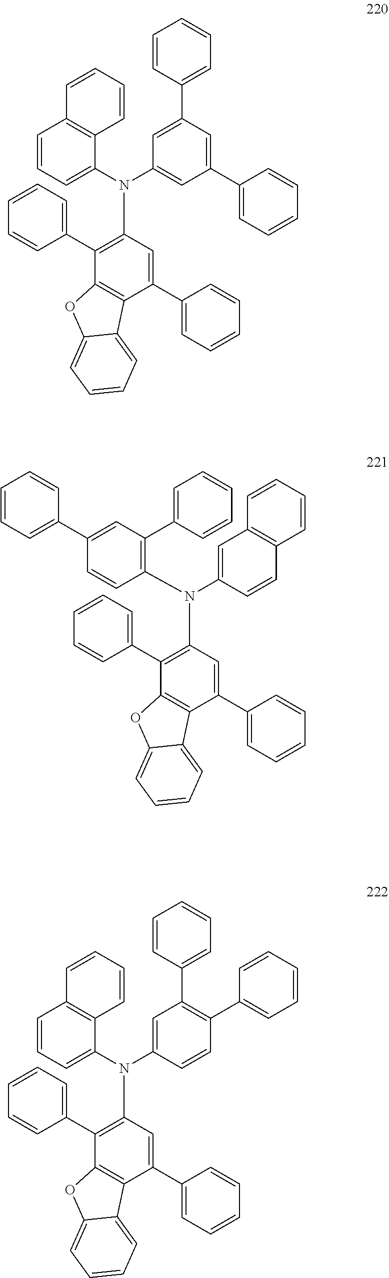 [SMILES: c1ccc(-c2cc(-c3ccccc3)cc(N(c3cc(-c4ccccc4)c4c(oc5ccccc54)c3-c3ccccc3)c3cccc4ccccc34)c2)cc1 ; c1ccc(-c2ccc(N(c3ccc4ccccc4c3)c3cc(-c4ccccc4)c4c(oc5ccccc54)c3-c3ccccc3)c(-c3ccccc3)c2)cc1 ; c1ccc(-c2ccc(N(c3cc(-c4ccccc4)c4c(oc5ccccc54)c3-c3ccccc3)c3cccc4ccccc34)cc2-c2ccccc2)cc1]

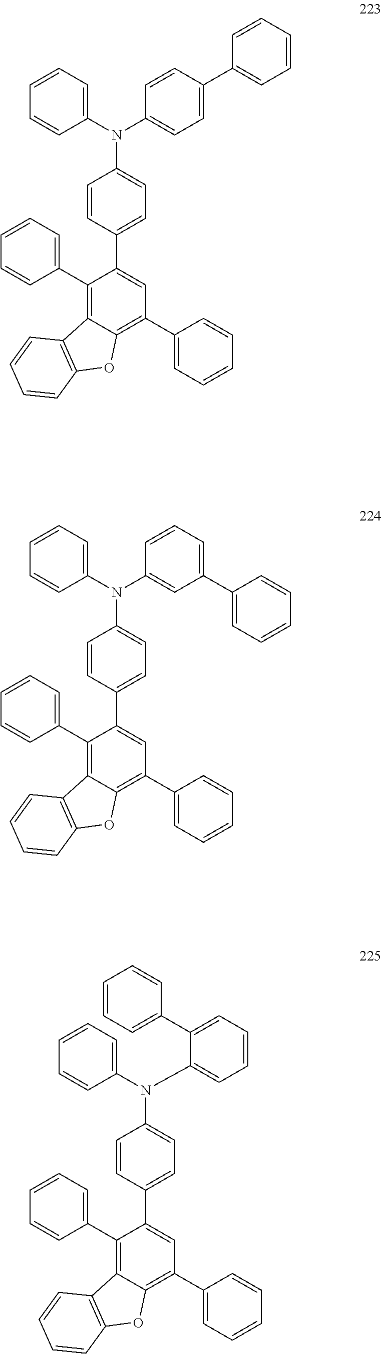 [SMILES: c1ccc(-c2ccc(N(c3ccccc3)c3ccc(-c4cc(-c5ccccc5)c5oc6ccccc6c5c4-c4ccccc4)cc3)cc2)cc1 ; c1ccc(-c2cccc(N(c3ccccc3)c3ccc(-c4cc(-c5ccccc5)c5oc6ccccc6c5c4-c4ccccc4)cc3)c2)cc1 ; c1ccc(-c2ccccc2N(c2ccccc2)c2ccc(-c3cc(-c4ccccc4)c4oc5ccccc5c4c3-c3ccccc3)cc2)cc1]

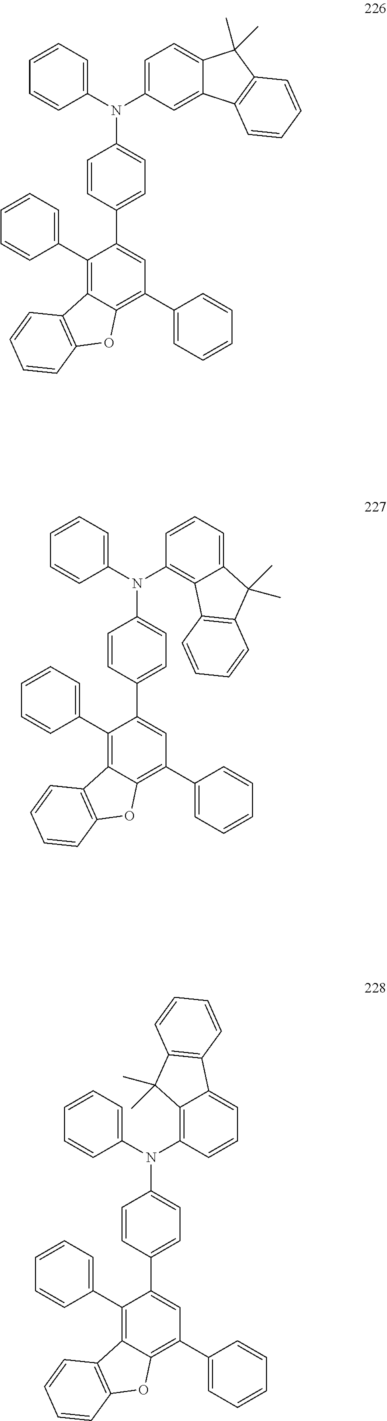 [SMILES: CC1(C)c2ccccc2-c2cc(N(c3ccccc3)c3ccc(-c4cc(-c5ccccc5)c5oc6ccccc6c5c4-c4ccccc4)cc3)ccc21 ; CC1(C)c2ccccc2-c2c(N(c3ccccc3)c3ccc(-c4cc(-c5ccccc5)c5oc6ccccc6c5c4-c4ccccc4)cc3)cccc21 ; CC1(C)c2ccccc2-c2cccc(N(c3ccccc3)c3ccc(-c4cc(-c5ccccc5)c5oc6ccccc6c5c4-c4ccccc4)cc3)c21]

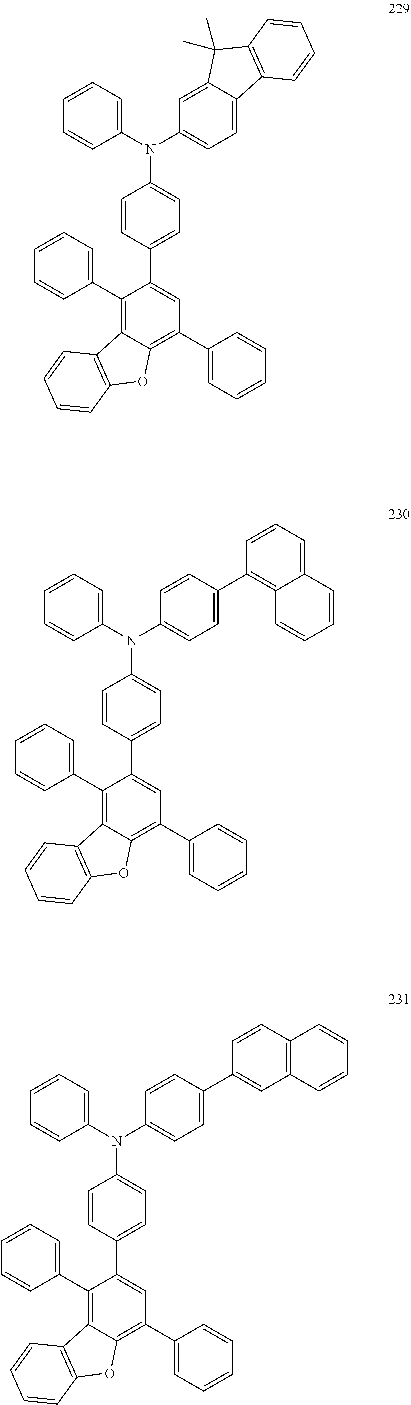 [SMILES: CC1(C)c2ccccc2-c2ccc(N(c3ccccc3)c3ccc(-c4cc(-c5ccccc5)c5oc6ccccc6c5c4-c4ccccc4)cc3)cc21 ; c1ccc(-c2cc(-c3ccc(N(c4ccccc4)c4ccc(-c5cccc6ccccc56)cc4)cc3)c(-c3ccccc3)c3c2oc2ccccc23)cc1 ; c1ccc(-c2cc(-c3ccc(N(c4ccccc4)c4ccc(-c5ccc6ccccc6c5)cc4)cc3)c(-c3ccccc3)c3c2oc2ccccc23)cc1]

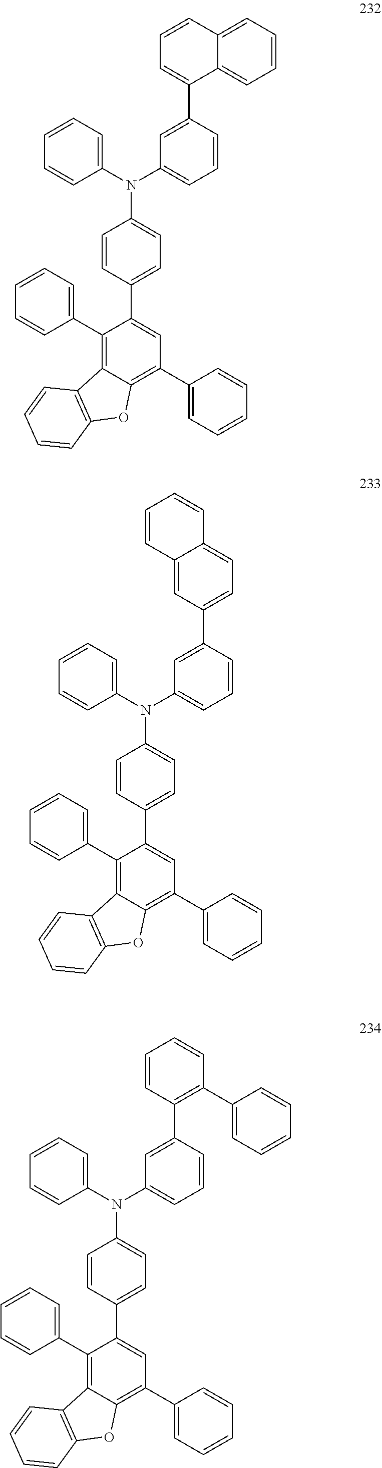 [SMILES: c1ccc(-c2cc(-c3ccc(N(c4ccccc4)c4cccc(-c5cccc6ccccc56)c4)cc3)c(-c3ccccc3)c3c2oc2ccccc23)cc1 ; c1ccc(-c2cc(-c3ccc(N(c4ccccc4)c4cccc(-c5ccc6ccccc6c5)c4)cc3)c(-c3ccccc3)c3c2oc2ccccc23)cc1 ; c1ccc(-c2ccccc2-c2cccc(N(c3ccccc3)c3ccc(-c4cc(-c5ccccc5)c5oc6ccccc6c5c4-c4ccccc4)cc3)c2)cc1]

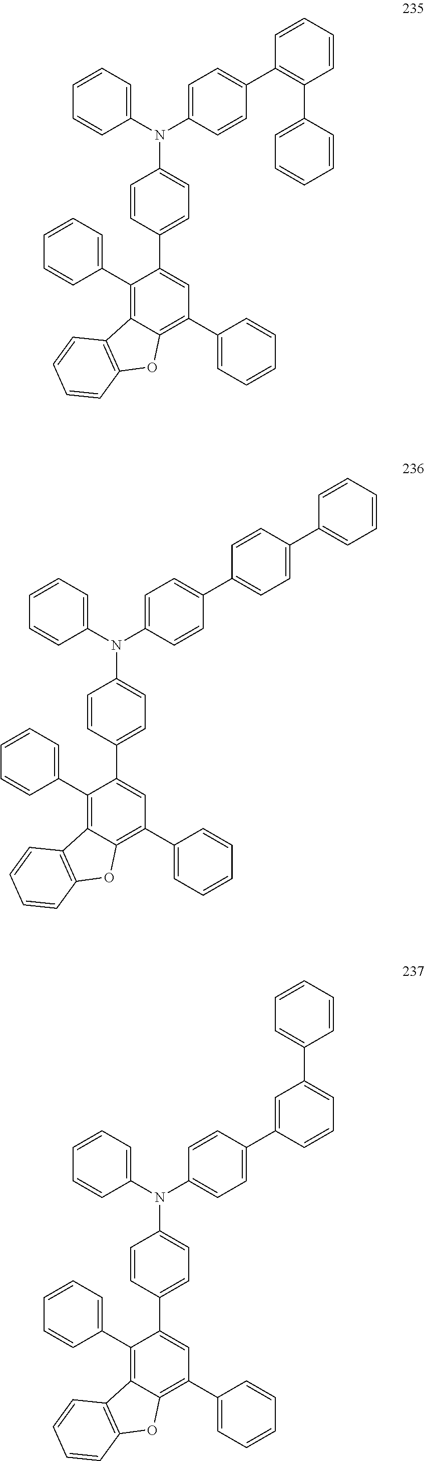 [SMILES: c1ccc(-c2ccccc2-c2ccc(N(c3ccccc3)c3ccc(-c4cc(-c5ccccc5)c5oc6ccccc6c5c4-c4ccccc4)cc3)cc2)cc1 ; c1ccc(-c2ccc(-c3ccc(N(c4ccccc4)c4ccc(-c5cc(-c6ccccc6)c6oc7ccccc7c6c5-c5ccccc5)cc4)cc3)cc2)cc1 ; c1ccc(-c2cccc(-c3ccc(N(c4ccccc4)c4ccc(-c5cc(-c6ccccc6)c6oc7ccccc7c6c5-c5ccccc5)cc4)cc3)c2)cc1]

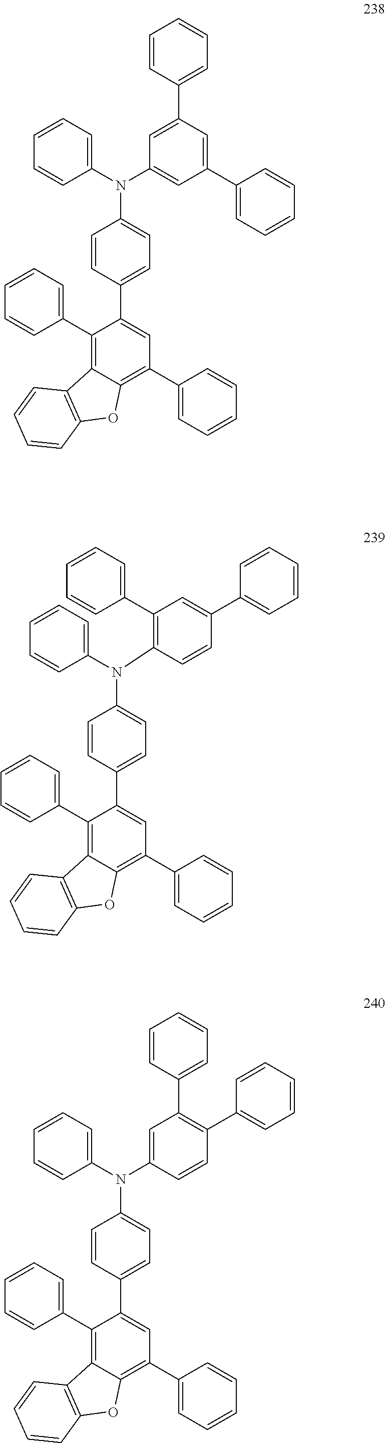 [SMILES: c1ccc(-c2cc(-c3ccccc3)cc(N(c3ccccc3)c3ccc(-c4cc(-c5ccccc5)c5oc6ccccc6c5c4-c4ccccc4)cc3)c2)cc1 ; c1ccc(-c2ccc(N(c3ccccc3)c3ccc(-c4cc(-c5ccccc5)c5oc6ccccc6c5c4-c4ccccc4)cc3)c(-c3ccccc3)c2)cc1 ; c1ccc(-c2ccc(N(c3ccccc3)c3ccc(-c4cc(-c5ccccc5)c5oc6ccccc6c5c4-c4ccccc4)cc3)cc2-c2ccccc2)cc1]

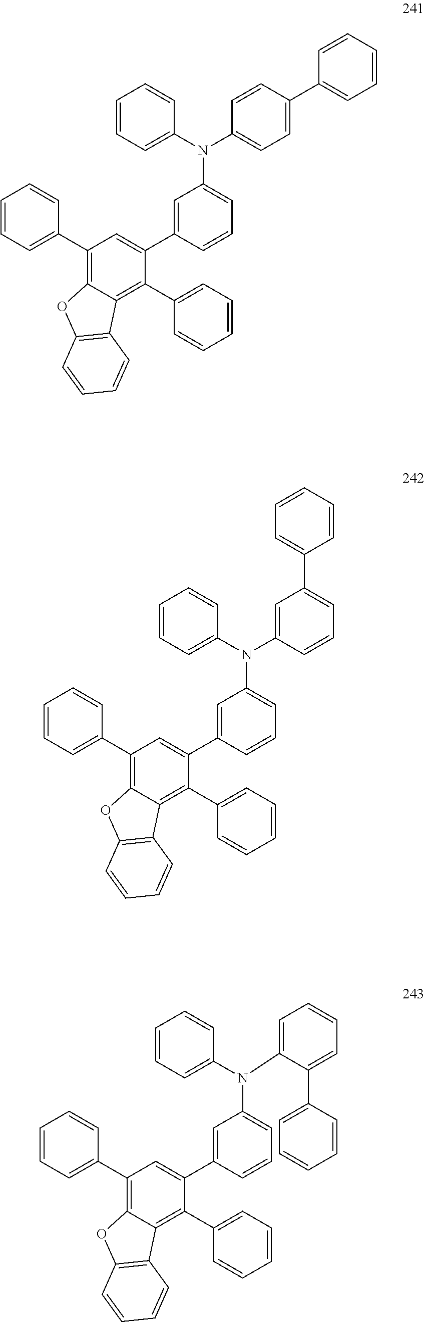 [SMILES: c1ccc(-c2ccc(N(c3ccccc3)c3cccc(-c4cc(-c5ccccc5)c5oc6ccccc6c5c4-c4ccccc4)c3)cc2)cc1 ; c1ccc(-c2cccc(N(c3ccccc3)c3cccc(-c4cc(-c5ccccc5)c5oc6ccccc6c5c4-c4ccccc4)c3)c2)cc1 ; c1ccc(-c2ccccc2N(c2ccccc2)c2cccc(-c3cc(-c4ccccc4)c4oc5ccccc5c4c3-c3ccccc3)c2)cc1]

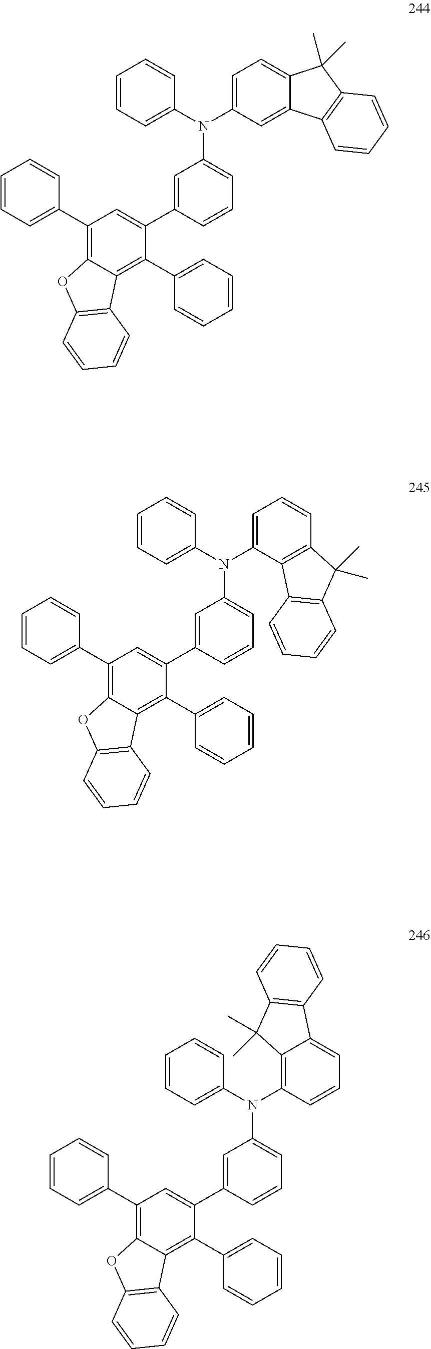 [SMILES: CC1(C)c2ccccc2-c2cc(N(c3ccccc3)c3cccc(-c4cc(-c5ccccc5)c5oc6ccccc6c5c4-c4ccccc4)c3)ccc21 ; CC1(C)c2ccccc2-c2c(N(c3ccccc3)c3cccc(-c4cc(-c5ccccc5)c5oc6ccccc6c5c4-c4ccccc4)c3)cccc21 ; CC1(C)c2ccccc2-c2cccc(N(c3ccccc3)c3cccc(-c4cc(-c5ccccc5)c5oc6ccccc6c5c4-c4ccccc4)c3)c21]

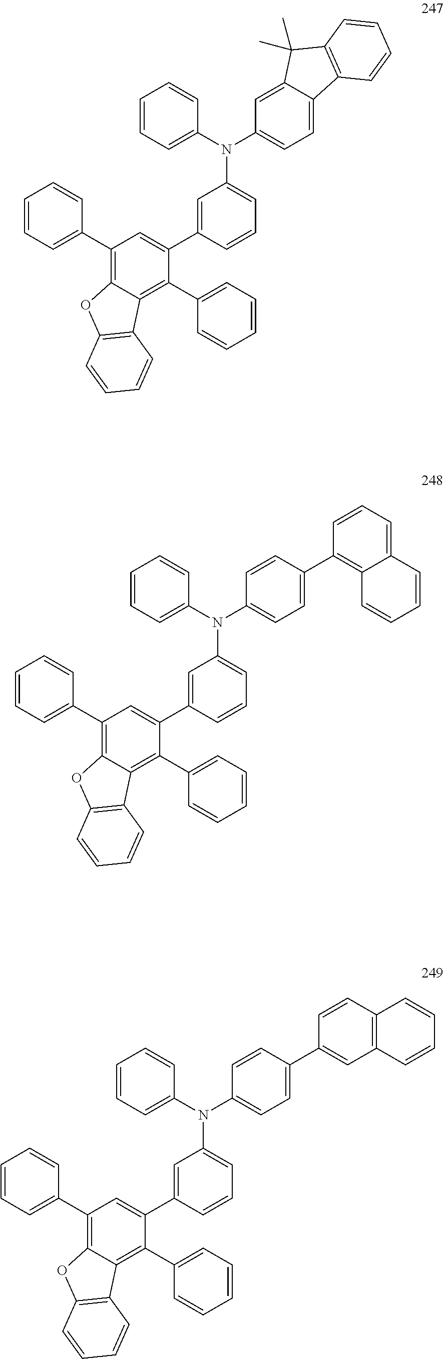 [SMILES: CC1(C)c2ccccc2-c2ccc(N(c3ccccc3)c3cccc(-c4cc(-c5ccccc5)c5oc6ccccc6c5c4-c4ccccc4)c3)cc21 ; c1ccc(-c2cc(-c3cccc(N(c4ccccc4)c4ccc(-c5cccc6ccccc56)cc4)c3)c(-c3ccccc3)c3c2oc2ccccc23)cc1 ; c1ccc(-c2cc(-c3cccc(N(c4ccccc4)c4ccc(-c5ccc6ccccc6c5)cc4)c3)c(-c3ccccc3)c3c2oc2ccccc23)cc1]

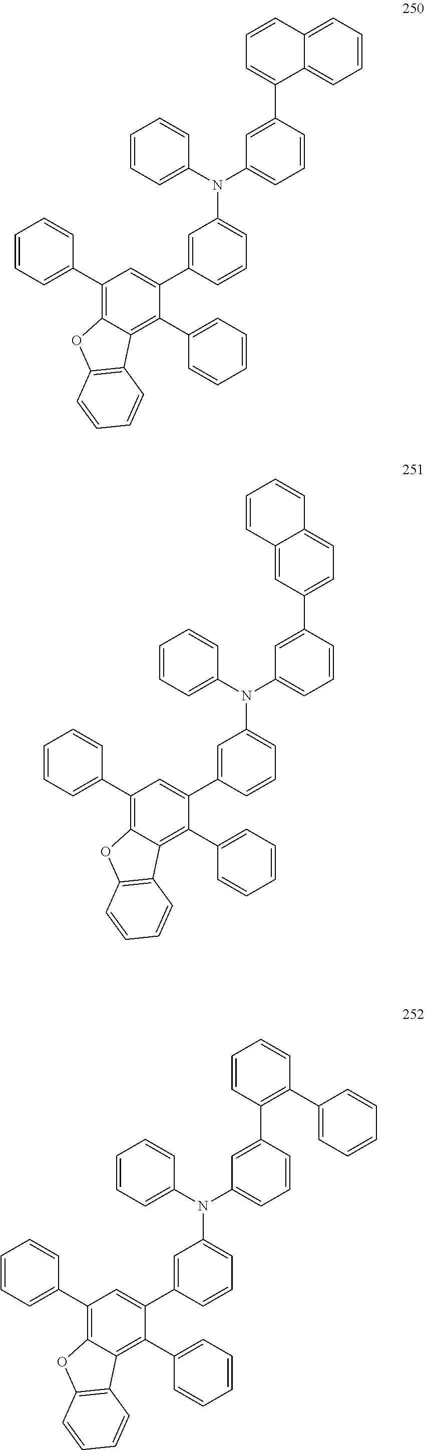 [SMILES: c1ccc(-c2cc(-c3cccc(N(c4ccccc4)c4cccc(-c5cccc6ccccc56)c4)c3)c(-c3ccccc3)c3c2oc2ccccc23)cc1 ; c1ccc(-c2cc(-c3cccc(N(c4ccccc4)c4cccc(-c5ccc6ccccc6c5)c4)c3)c(-c3ccccc3)c3c2oc2ccccc23)cc1 ; c1ccc(-c2ccccc2-c2cccc(N(c3ccccc3)c3cccc(-c4cc(-c5ccccc5)c5oc6ccccc6c5c4-c4ccccc4)c3)c2)cc1]

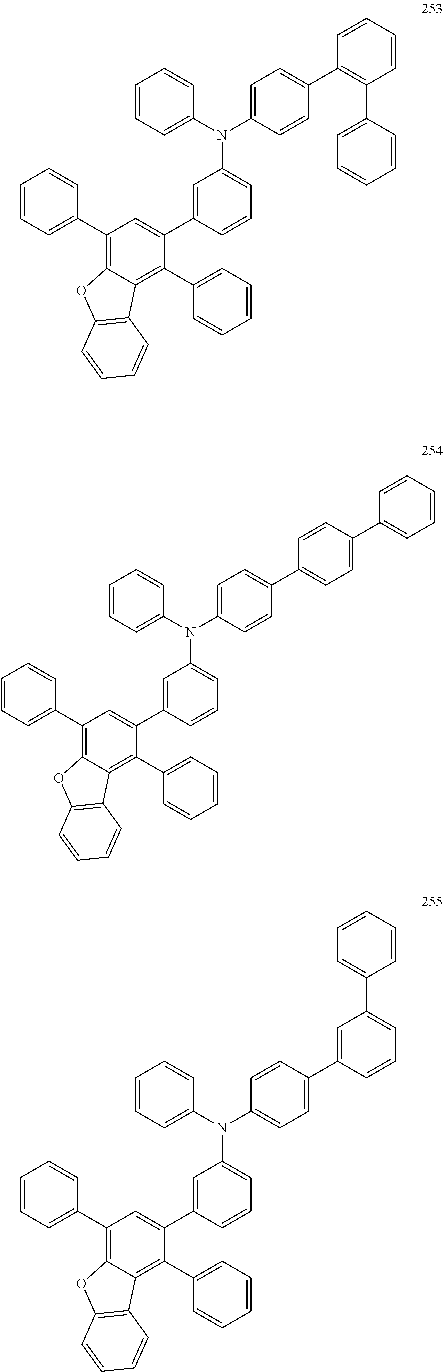 [SMILES: c1ccc(-c2ccccc2-c2ccc(N(c3ccccc3)c3cccc(-c4cc(-c5ccccc5)c5oc6ccccc6c5c4-c4ccccc4)c3)cc2)cc1 ; c1ccc(-c2ccc(-c3ccc(N(c4ccccc4)c4cccc(-c5cc(-c6ccccc6)c6oc7ccccc7c6c5-c5ccccc5)c4)cc3)cc2)cc1 ; c1ccc(-c2cccc(-c3ccc(N(c4ccccc4)c4cccc(-c5cc(-c6ccccc6)c6oc7ccccc7c6c5-c5ccccc5)c4)cc3)c2)cc1]

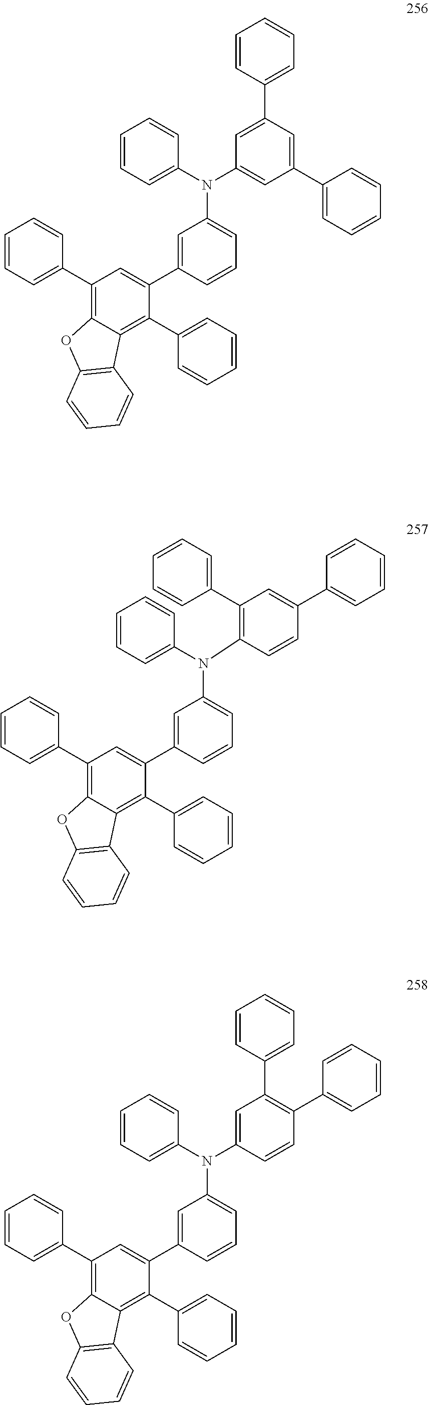 [SMILES: c1ccc(-c2cc(-c3ccccc3)cc(N(c3ccccc3)c3cccc(-c4cc(-c5ccccc5)c5oc6ccccc6c5c4-c4ccccc4)c3)c2)cc1 ; c1ccc(-c2ccc(N(c3ccccc3)c3cccc(-c4cc(-c5ccccc5)c5oc6ccccc6c5c4-c4ccccc4)c3)c(-c3ccccc3)c2)cc1 ; c1ccc(-c2ccc(N(c3ccccc3)c3cccc(-c4cc(-c5ccccc5)c5oc6ccccc6c5c4-c4ccccc4)c3)cc2-c2ccccc2)cc1]

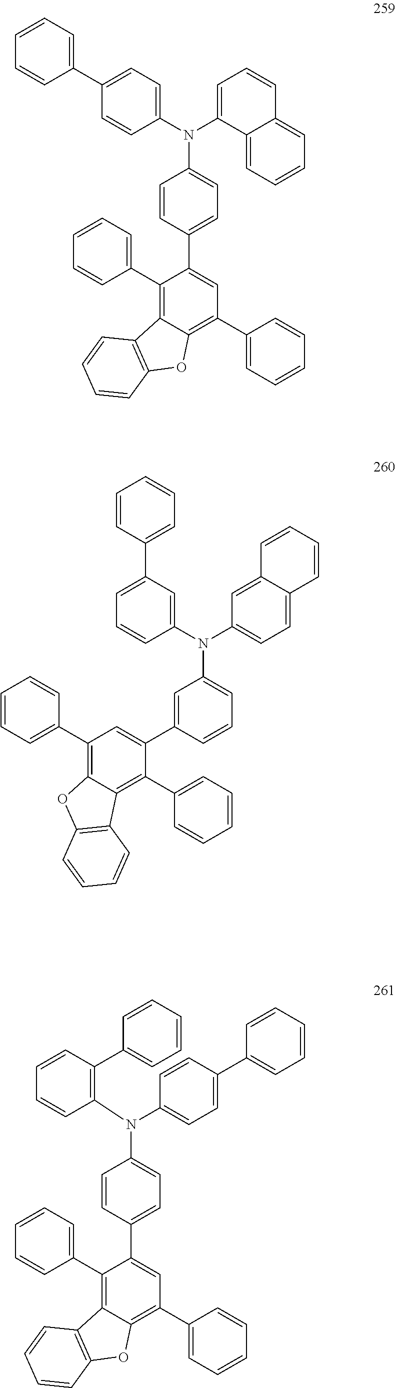 [SMILES: c1ccc(-c2ccc(N(c3ccc(-c4cc(-c5ccccc5)c5oc6ccccc6c5c4-c4ccccc4)cc3)c3cccc4ccccc34)cc2)cc1 ; c1ccc(-c2cccc(N(c3cccc(-c4cc(-c5ccccc5)c5oc6ccccc6c5c4-c4ccccc4)c3)c3ccc4ccccc4c3)c2)cc1 ; c1ccc(-c2ccc(N(c3ccc(-c4cc(-c5ccccc5)c5oc6ccccc6c5c4-c4ccccc4)cc3)c3ccccc3-c3ccccc3)cc2)cc1]

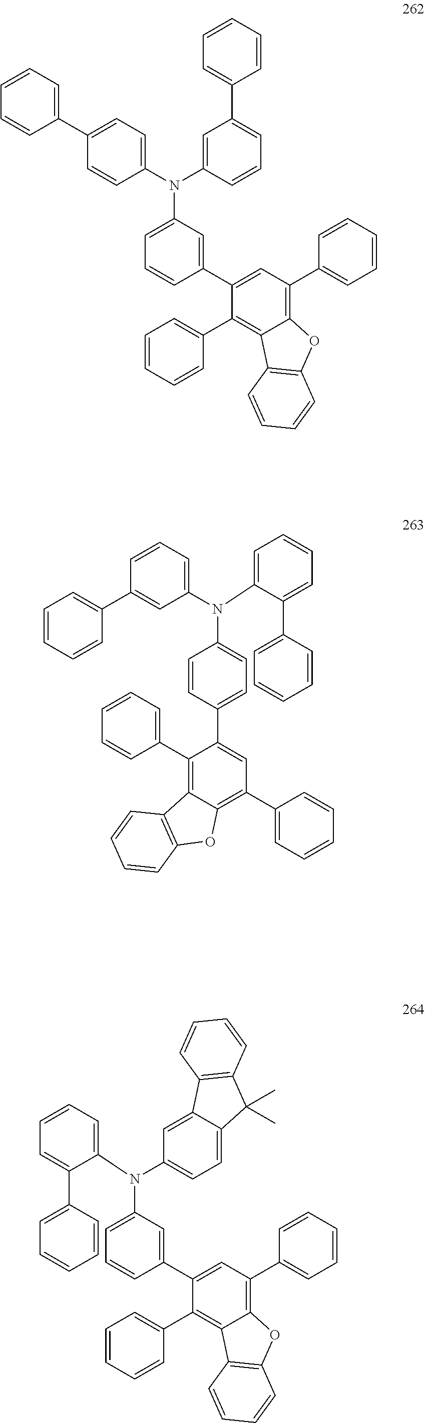 [SMILES: c1ccc(-c2ccc(N(c3cccc(-c4ccccc4)c3)c3cccc(-c4cc(-c5ccccc5)c5oc6ccccc6c5c4-c4ccccc4)c3)cc2)cc1 ; c1ccc(-c2cccc(N(c3ccc(-c4cc(-c5ccccc5)c5oc6ccccc6c5c4-c4ccccc4)cc3)c3ccccc3-c3ccccc3)c2)cc1 ; CC1(C)c2ccccc2-c2cc(N(c3cccc(-c4cc(-c5ccccc5)c5oc6ccccc6c5c4-c4ccccc4)c3)c3ccccc3-c3ccccc3)ccc21]

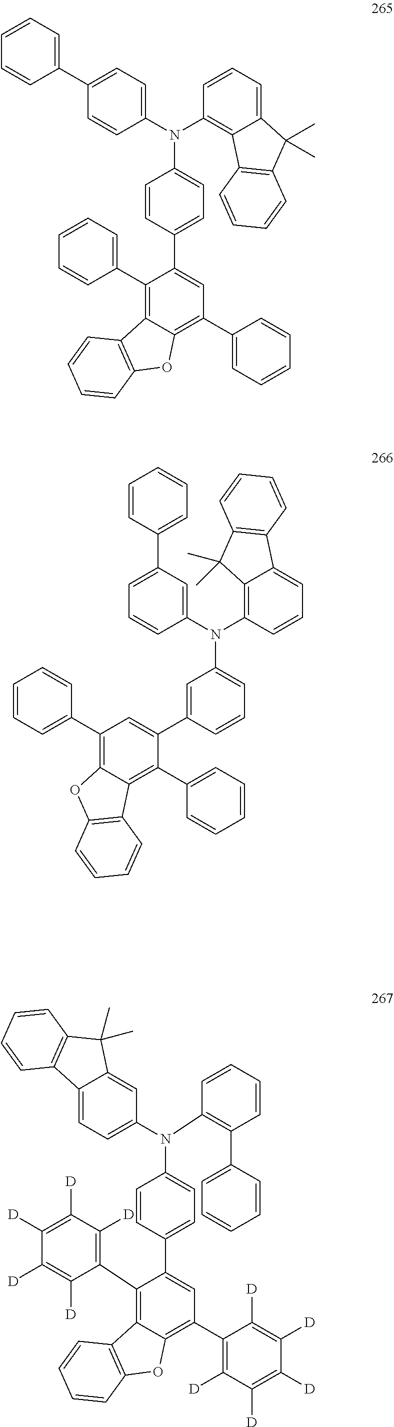 [SMILES: CC1(C)c2ccccc2-c2c(N(c3ccc(-c4ccccc4)cc3)c3ccc(-c4cc(-c5ccccc5)c5oc6ccccc6c5c4-c4ccccc4)cc3)cccc21 ; CC1(C)c2ccccc2-c2cccc(N(c3cccc(-c4ccccc4)c3)c3cccc(-c4cc(-c5ccccc5)c5oc6ccccc6c5c4-c4ccccc4)c3)c21 ; [2H]c1c([2H])c([2H])c(-c2cc(-c3ccc(N(c4ccc5c(c4)C(C)(C)c4ccccc4-5)c4ccccc4-c4ccccc4)cc3)c(-c3c([2H])c([2H])c([2H])c([2H])c3[2H])c3c2oc2ccccc23)c([2H])c1[2H]]

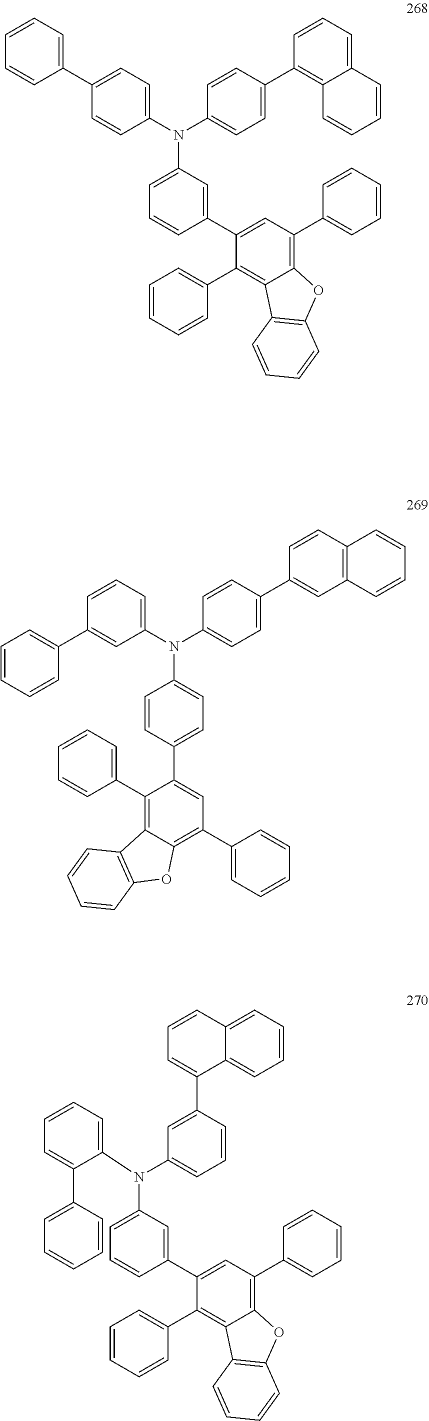 [SMILES: c1ccc(-c2ccc(N(c3ccc(-c4cccc5ccccc45)cc3)c3cccc(-c4cc(-c5ccccc5)c5oc6ccccc6c5c4-c4ccccc4)c3)cc2)cc1 ; c1ccc(-c2cccc(N(c3ccc(-c4ccc5ccccc5c4)cc3)c3ccc(-c4cc(-c5ccccc5)c5oc6ccccc6c5c4-c4ccccc4)cc3)c2)cc1 ; c1ccc(-c2ccccc2N(c2cccc(-c3cc(-c4ccccc4)c4oc5ccccc5c4c3-c3ccccc3)c2)c2cccc(-c3cccc4ccccc34)c2)cc1]

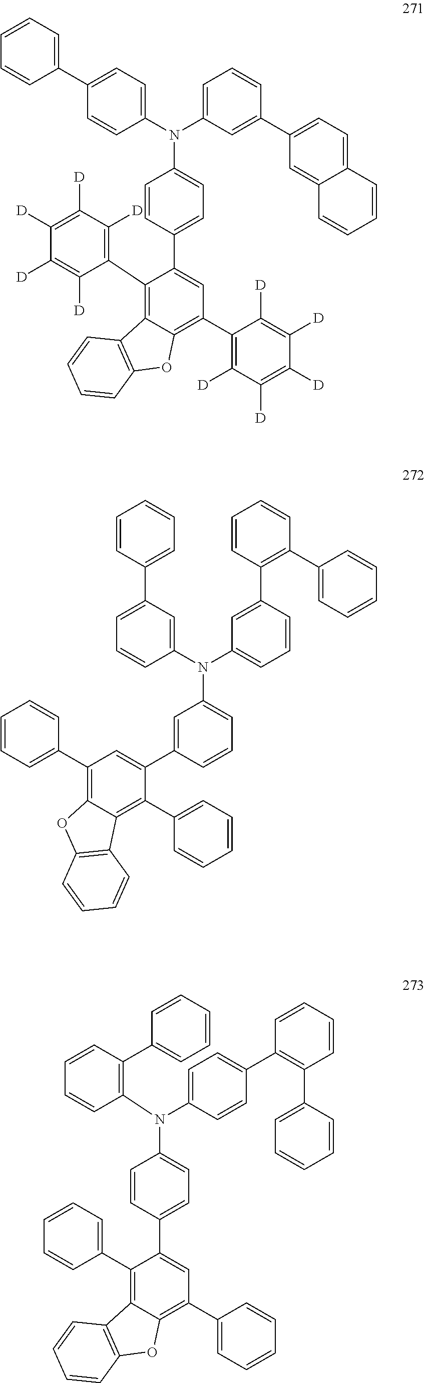 [SMILES: [2H]c1c([2H])c([2H])c(-c2cc(-c3ccc(N(c4ccc(-c5ccccc5)cc4)c4cccc(-c5ccc6ccccc6c5)c4)cc3)c(-c3c([2H])c([2H])c([2H])c([2H])c3[2H])c3c2oc2ccccc23)c([2H])c1[2H] ; c1ccc(-c2cccc(N(c3cccc(-c4ccccc4-c4ccccc4)c3)c3cccc(-c4cc(-c5ccccc5)c5oc6ccccc6c5c4-c4ccccc4)c3)c2)cc1 ; c1ccc(-c2ccccc2-c2ccc(N(c3ccc(-c4cc(-c5ccccc5)c5oc6ccccc6c5c4-c4ccccc4)cc3)c3ccccc3-c3ccccc3)cc2)cc1]

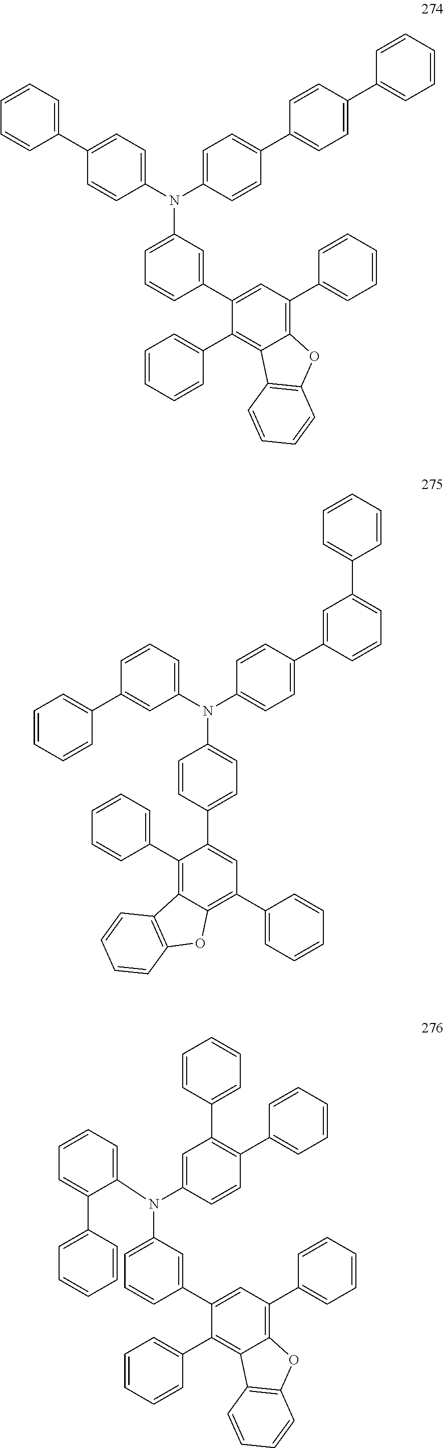 [SMILES: c1ccc(-c2ccc(-c3ccc(N(c4ccc(-c5ccccc5)cc4)c4cccc(-c5cc(-c6ccccc6)c6oc7ccccc7c6c5-c5ccccc5)c4)cc3)cc2)cc1 ; c1ccc(-c2cccc(-c3ccc(N(c4ccc(-c5cc(-c6ccccc6)c6oc7ccccc7c6c5-c5ccccc5)cc4)c4cccc(-c5ccccc5)c4)cc3)c2)cc1 ; c1ccc(-c2ccc(N(c3cccc(-c4cc(-c5ccccc5)c5oc6ccccc6c5c4-c4ccccc4)c3)c3ccccc3-c3ccccc3)cc2-c2ccccc2)cc1]

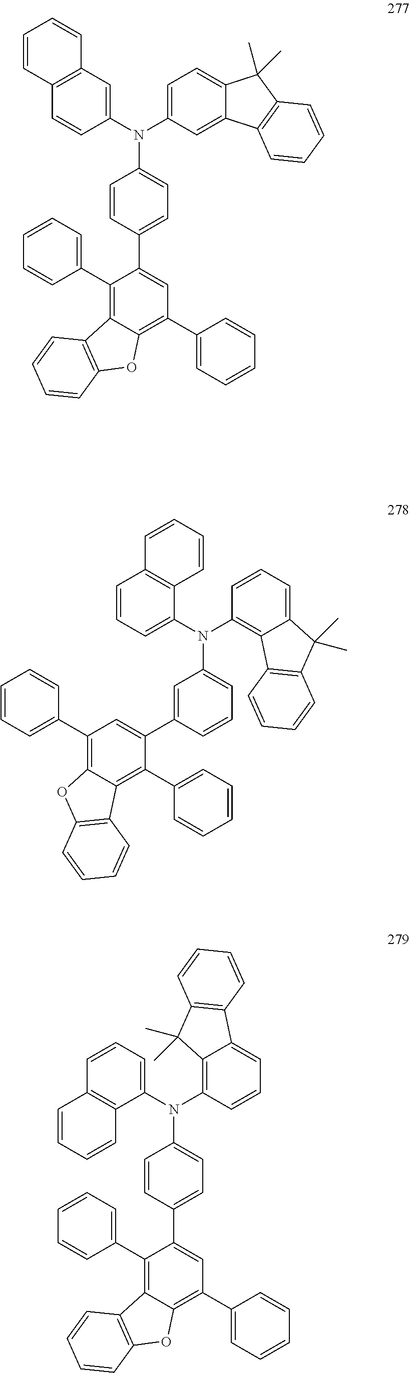 [SMILES: CC1(C)c2ccccc2-c2cc(N(c3ccc(-c4cc(-c5ccccc5)c5oc6ccccc6c5c4-c4ccccc4)cc3)c3ccc4ccccc4c3)ccc21 ; CC1(C)c2ccccc2-c2c(N(c3cccc(-c4cc(-c5ccccc5)c5oc6ccccc6c5c4-c4ccccc4)c3)c3cccc4ccccc34)cccc21 ; CC1(C)c2ccccc2-c2cccc(N(c3ccc(-c4cc(-c5ccccc5)c5oc6ccccc6c5c4-c4ccccc4)cc3)c3cccc4ccccc34)c21]

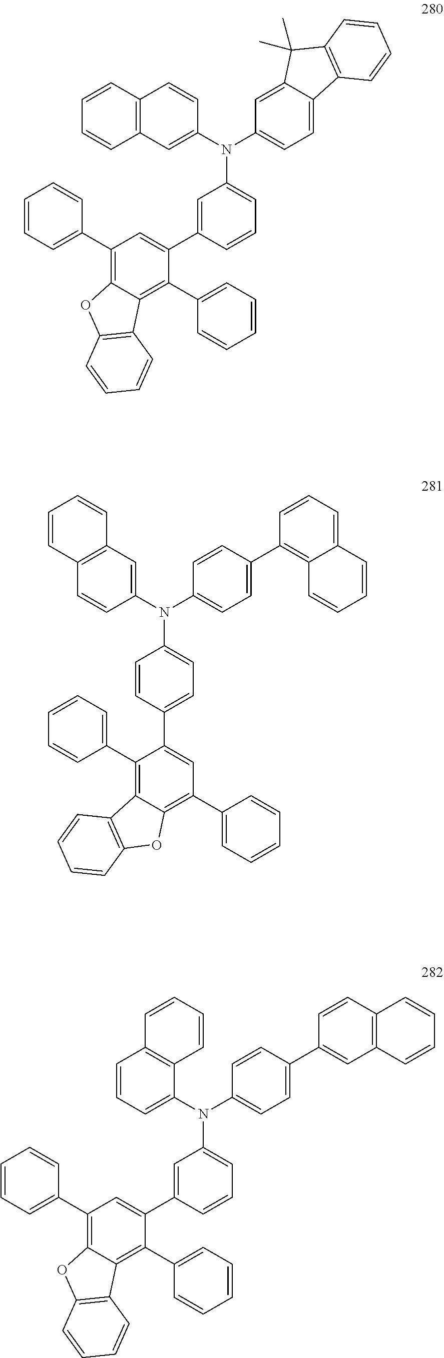 [SMILES: CC1(C)c2ccccc2-c2ccc(N(c3cccc(-c4cc(-c5ccccc5)c5oc6ccccc6c5c4-c4ccccc4)c3)c3ccc4ccccc4c3)cc21 ; c1ccc(-c2cc(-c3ccc(N(c4ccc(-c5cccc6ccccc56)cc4)c4ccc5ccccc5c4)cc3)c(-c3ccccc3)c3c2oc2ccccc23)cc1 ; c1ccc(-c2cc(-c3cccc(N(c4ccc(-c5ccc6ccccc6c5)cc4)c4cccc5ccccc45)c3)c(-c3ccccc3)c3c2oc2ccccc23)cc1]

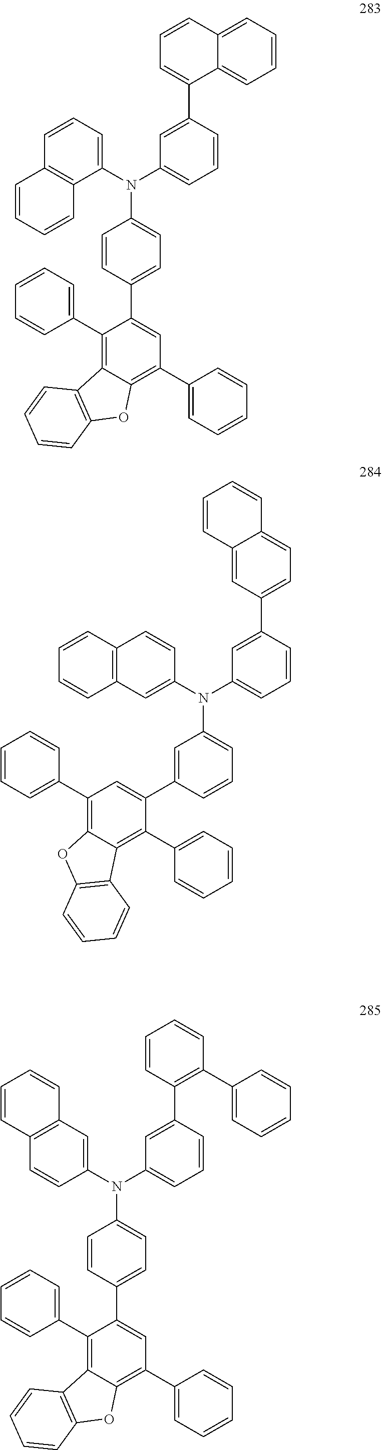 [SMILES: c1ccc(-c2cc(-c3ccc(N(c4cccc(-c5cccc6ccccc56)c4)c4cccc5ccccc45)cc3)c(-c3ccccc3)c3c2oc2ccccc23)cc1 ; c1ccc(-c2cc(-c3cccc(N(c4cccc(-c5ccc6ccccc6c5)c4)c4ccc5ccccc5c4)c3)c(-c3ccccc3)c3c2oc2ccccc23)cc1 ; c1ccc(-c2ccccc2-c2cccc(N(c3ccc(-c4cc(-c5ccccc5)c5oc6ccccc6c5c4-c4ccccc4)cc3)c3ccc4ccccc4c3)c2)cc1]

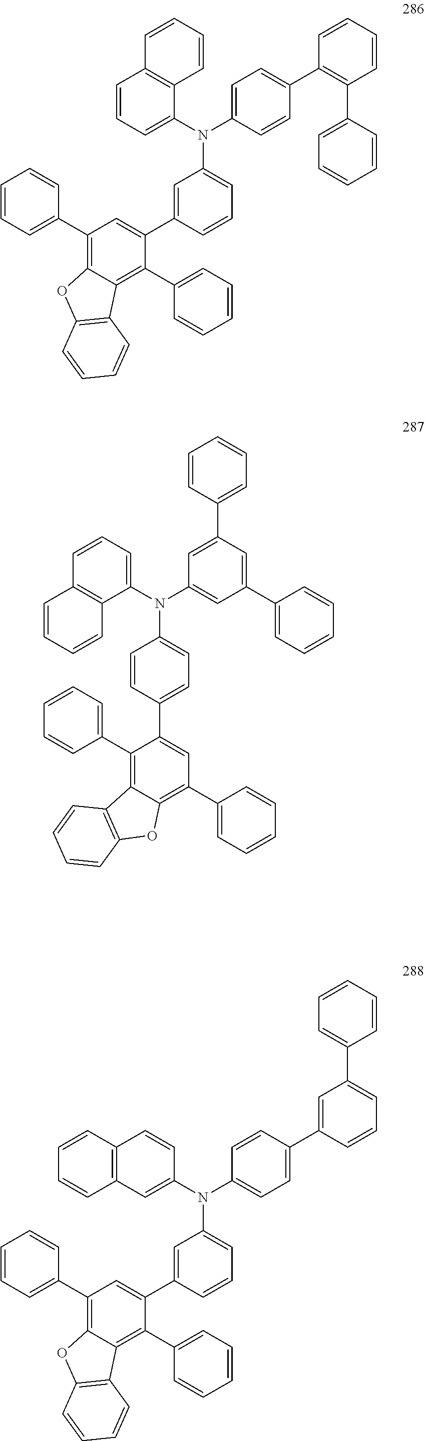 [SMILES: c1ccc(-c2ccccc2-c2ccc(N(c3cccc(-c4cc(-c5ccccc5)c5oc6ccccc6c5c4-c4ccccc4)c3)c3cccc4ccccc34)cc2)cc1 ; c1ccc(-c2cc(-c3ccccc3)cc(N(c3ccc(-c4cc(-c5ccccc5)c5oc6ccccc6c5c4-c4ccccc4)cc3)c3cccc4ccccc34)c2)cc1 ; c1ccc(-c2cccc(-c3ccc(N(c4cccc(-c5cc(-c6ccccc6)c6oc7ccccc7c6c5-c5ccccc5)c4)c4ccc5ccccc5c4)cc3)c2)cc1]

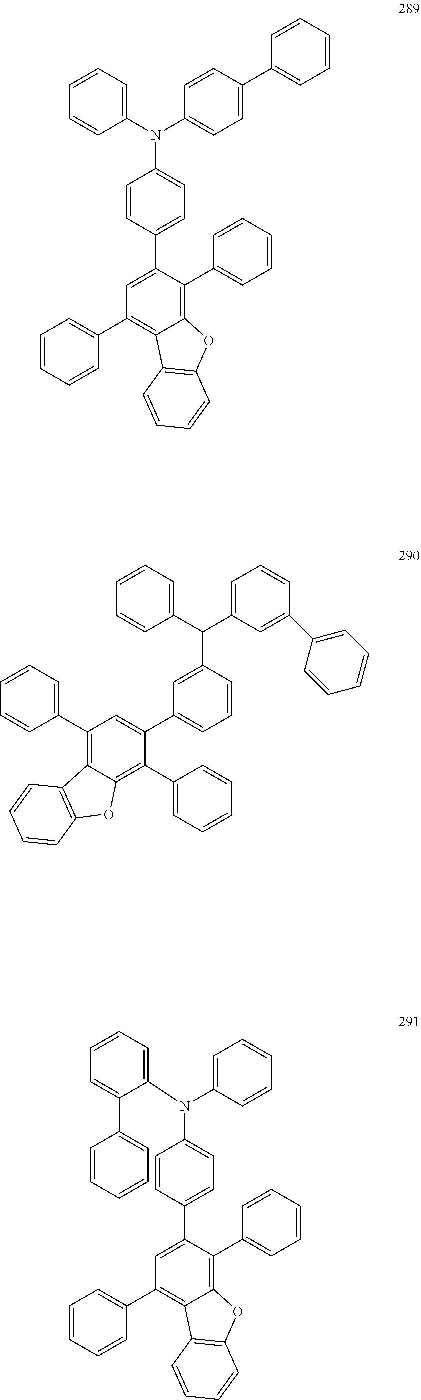 [SMILES: c1ccc(-c2ccc(N(c3ccccc3)c3ccc(-c4cc(-c5ccccc5)c5c(oc6ccccc65)c4-c4ccccc4)cc3)cc2)cc1 ; c1ccc(-c2cccc(C(c3ccccc3)c3cccc(-c4cc(-c5ccccc5)c5c(oc6ccccc65)c4-c4ccccc4)c3)c2)cc1 ; c1ccc(-c2ccccc2N(c2ccccc2)c2ccc(-c3cc(-c4ccccc4)c4c(oc5ccccc54)c3-c3ccccc3)cc2)cc1]

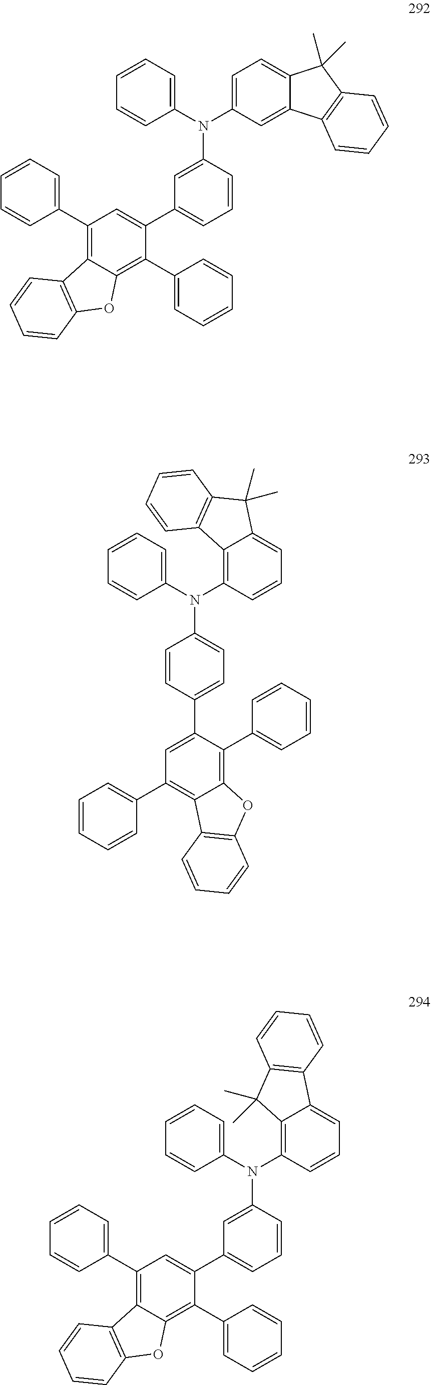 [SMILES: CC1(C)c2ccccc2-c2cc(N(c3ccccc3)c3cccc(-c4cc(-c5ccccc5)c5c(oc6ccccc65)c4-c4ccccc4)c3)ccc21 ; CC1(C)c2ccccc2-c2c(N(c3ccccc3)c3ccc(-c4cc(-c5ccccc5)c5c(oc6ccccc65)c4-c4ccccc4)cc3)cccc21 ; CC1(C)c2ccccc2-c2cccc(N(c3ccccc3)c3cccc(-c4cc(-c5ccccc5)c5c(oc6ccccc65)c4-c4ccccc4)c3)c21]

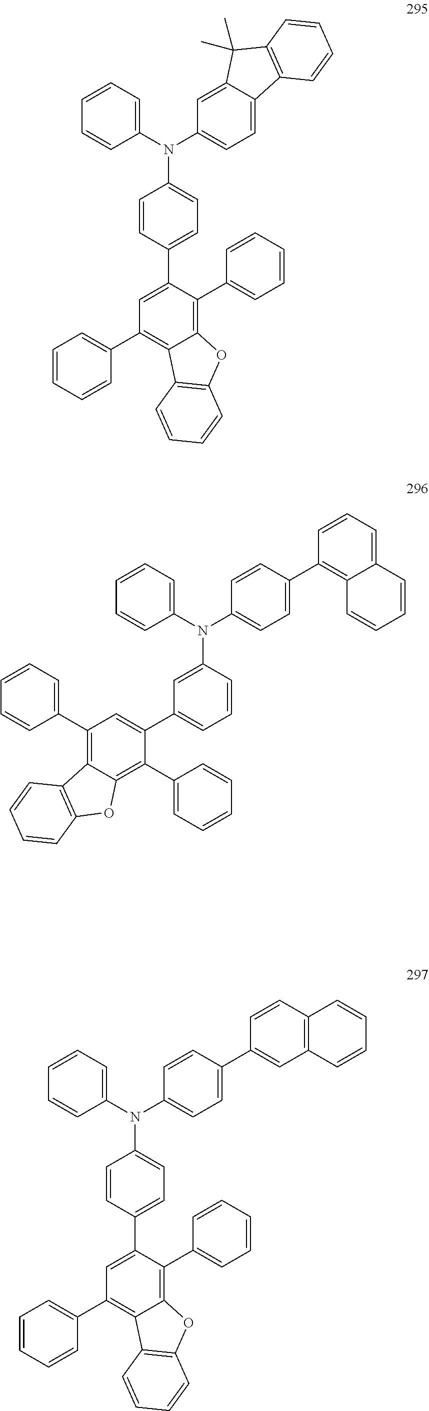 [SMILES: CC1(C)c2ccccc2-c2ccc(N(c3ccccc3)c3ccc(-c4cc(-c5ccccc5)c5c(oc6ccccc65)c4-c4ccccc4)cc3)cc21 ; c1ccc(-c2c(-c3cccc(N(c4ccccc4)c4ccc(-c5cccc6ccccc56)cc4)c3)cc(-c3ccccc3)c3c2oc2ccccc23)cc1 ; c1ccc(-c2c(-c3ccc(N(c4ccccc4)c4ccc(-c5ccc6ccccc6c5)cc4)cc3)cc(-c3ccccc3)c3c2oc2ccccc23)cc1]

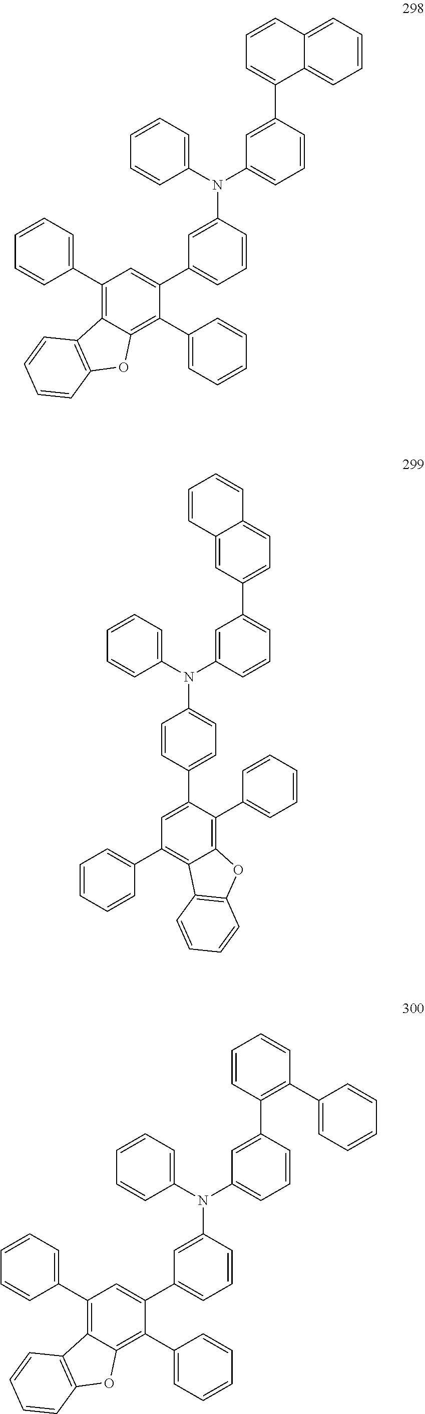 [SMILES: c1ccc(-c2c(-c3cccc(N(c4ccccc4)c4cccc(-c5cccc6ccccc56)c4)c3)cc(-c3ccccc3)c3c2oc2ccccc23)cc1 ; c1ccc(-c2c(-c3ccc(N(c4ccccc4)c4cccc(-c5ccc6ccccc6c5)c4)cc3)cc(-c3ccccc3)c3c2oc2ccccc23)cc1 ; c1ccc(-c2ccccc2-c2cccc(N(c3ccccc3)c3cccc(-c4cc(-c5ccccc5)c5c(oc6ccccc65)c4-c4ccccc4)c3)c2)cc1]

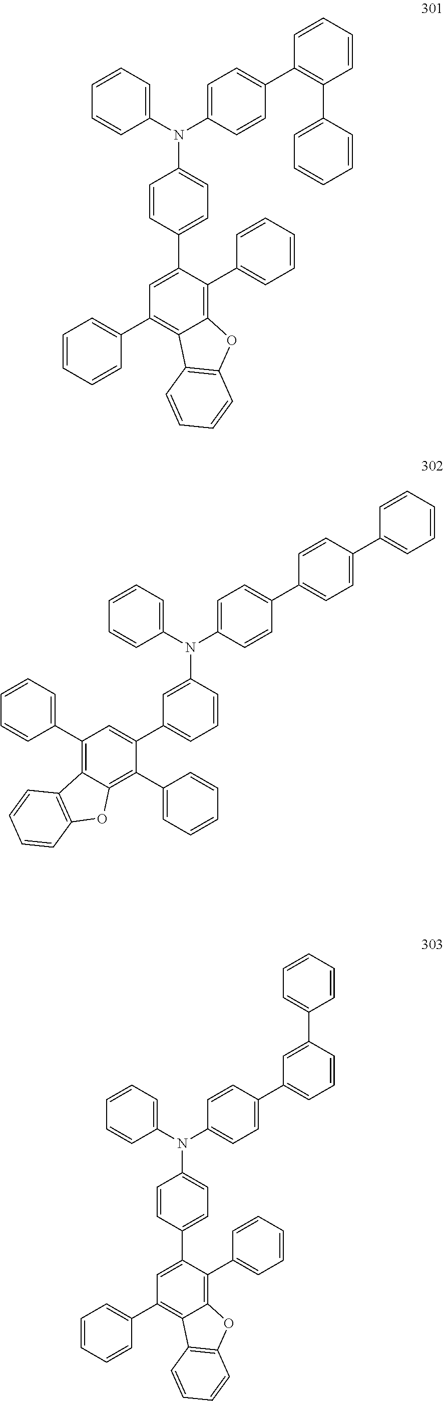 [SMILES: c1ccc(-c2ccccc2-c2ccc(N(c3ccccc3)c3ccc(-c4cc(-c5ccccc5)c5c(oc6ccccc65)c4-c4ccccc4)cc3)cc2)cc1 ; c1ccc(-c2ccc(-c3ccc(N(c4ccccc4)c4cccc(-c5cc(-c6ccccc6)c6c(oc7ccccc76)c5-c5ccccc5)c4)cc3)cc2)cc1 ; c1ccc(-c2cccc(-c3ccc(N(c4ccccc4)c4ccc(-c5cc(-c6ccccc6)c6c(oc7ccccc76)c5-c5ccccc5)cc4)cc3)c2)cc1]

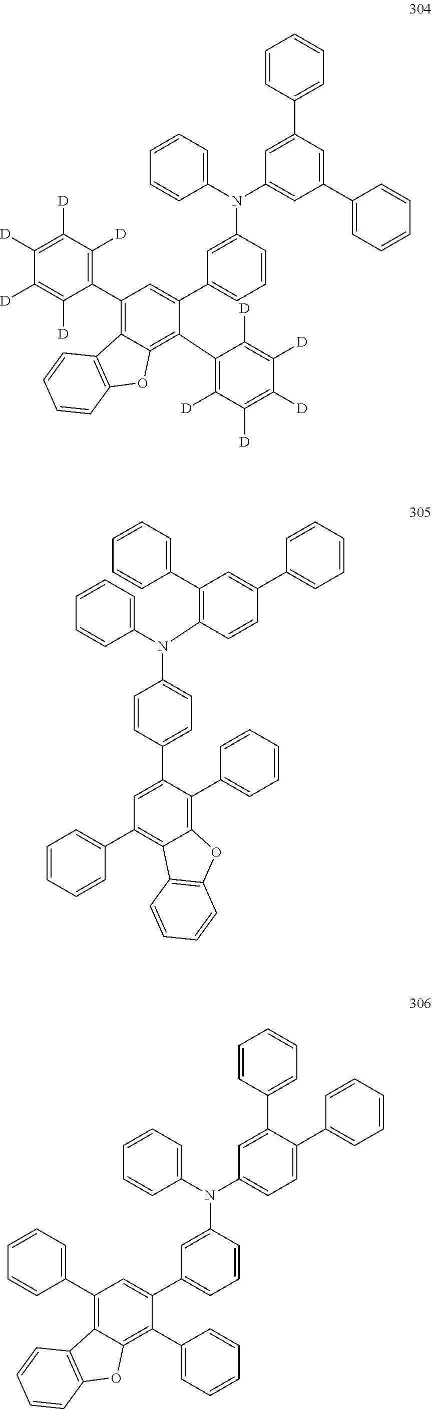 [SMILES: [2H]c1c([2H])c([2H])c(-c2c(-c3cccc(N(c4ccccc4)c4cc(-c5ccccc5)cc(-c5ccccc5)c4)c3)cc(-c3c([2H])c([2H])c([2H])c([2H])c3[2H])c3c2oc2ccccc23)c([2H])c1[2H] ; c1ccc(-c2ccc(N(c3ccccc3)c3ccc(-c4cc(-c5ccccc5)c5c(oc6ccccc65)c4-c4ccccc4)cc3)c(-c3ccccc3)c2)cc1 ; c1ccc(-c2ccc(N(c3ccccc3)c3cccc(-c4cc(-c5ccccc5)c5c(oc6ccccc65)c4-c4ccccc4)c3)cc2-c2ccccc2)cc1]

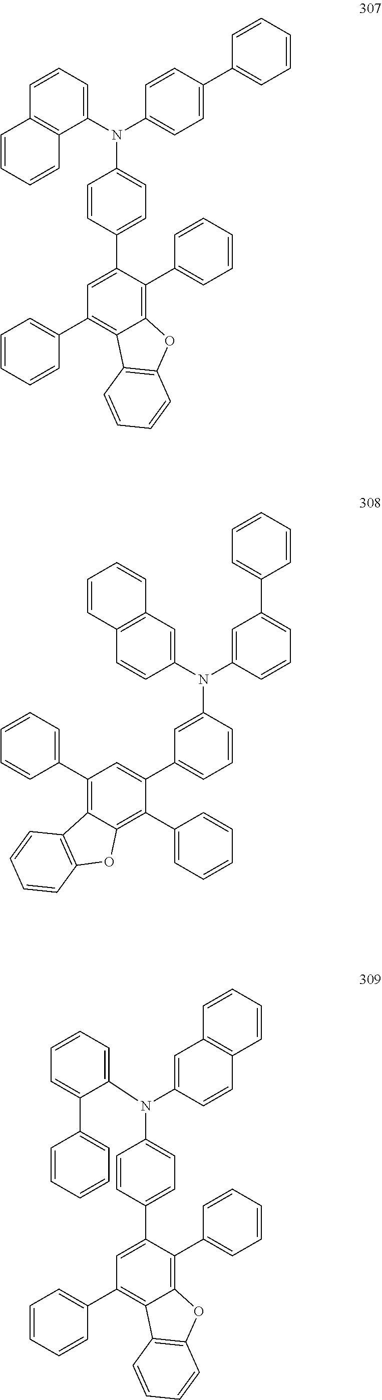 [SMILES: c1ccc(-c2ccc(N(c3ccc(-c4cc(-c5ccccc5)c5c(oc6ccccc65)c4-c4ccccc4)cc3)c3cccc4ccccc34)cc2)cc1 ; c1ccc(-c2cccc(N(c3cccc(-c4cc(-c5ccccc5)c5c(oc6ccccc65)c4-c4ccccc4)c3)c3ccc4ccccc4c3)c2)cc1 ; c1ccc(-c2ccccc2N(c2ccc(-c3cc(-c4ccccc4)c4c(oc5ccccc54)c3-c3ccccc3)cc2)c2ccc3ccccc3c2)cc1]

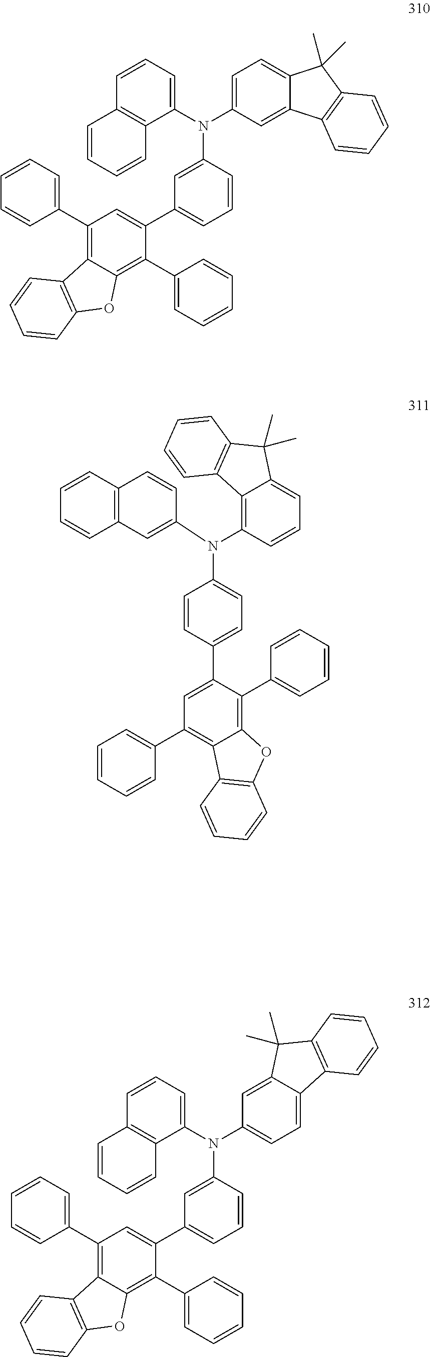 [SMILES: CC1(C)c2ccccc2-c2cc(N(c3cccc(-c4cc(-c5ccccc5)c5c(oc6ccccc65)c4-c4ccccc4)c3)c3cccc4ccccc34)ccc21 ; CC1(C)c2ccccc2-c2c(N(c3ccc(-c4cc(-c5ccccc5)c5c(oc6ccccc65)c4-c4ccccc4)cc3)c3ccc4ccccc4c3)cccc21 ; CC1(C)c2ccccc2-c2ccc(N(c3cccc(-c4cc(-c5ccccc5)c5c(oc6ccccc65)c4-c4ccccc4)c3)c3cccc4ccccc34)cc21]

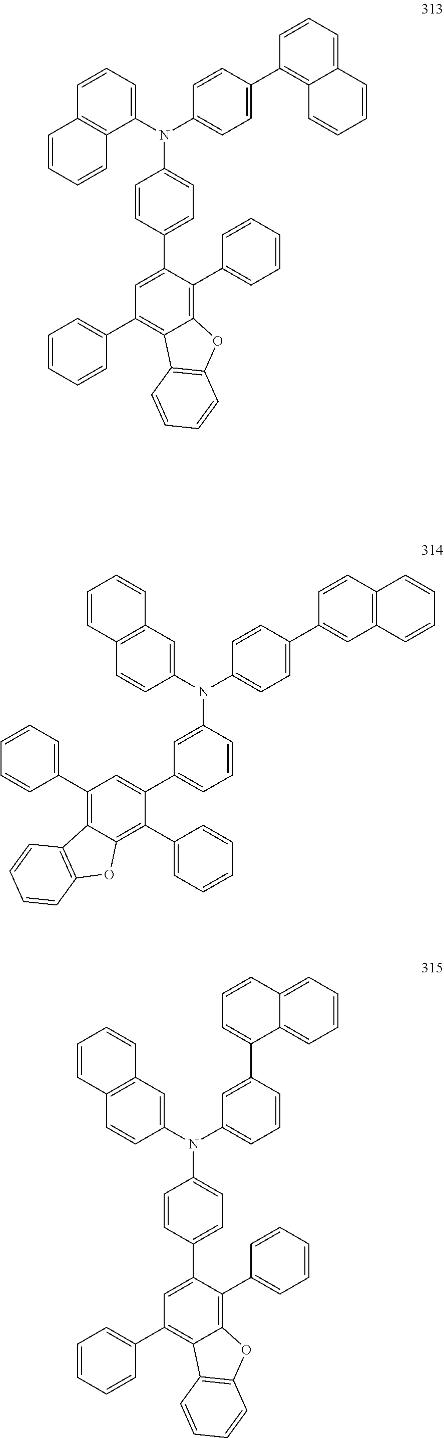 [SMILES: c1ccc(-c2c(-c3ccc(N(c4ccc(-c5cccc6ccccc56)cc4)c4cccc5ccccc45)cc3)cc(-c3ccccc3)c3c2oc2ccccc23)cc1 ; c1ccc(-c2c(-c3cccc(N(c4ccc(-c5ccc6ccccc6c5)cc4)c4ccc5ccccc5c4)c3)cc(-c3ccccc3)c3c2oc2ccccc23)cc1 ; c1ccc(-c2c(-c3ccc(N(c4cccc(-c5cccc6ccccc56)c4)c4ccc5ccccc5c4)cc3)cc(-c3ccccc3)c3c2oc2ccccc23)cc1]

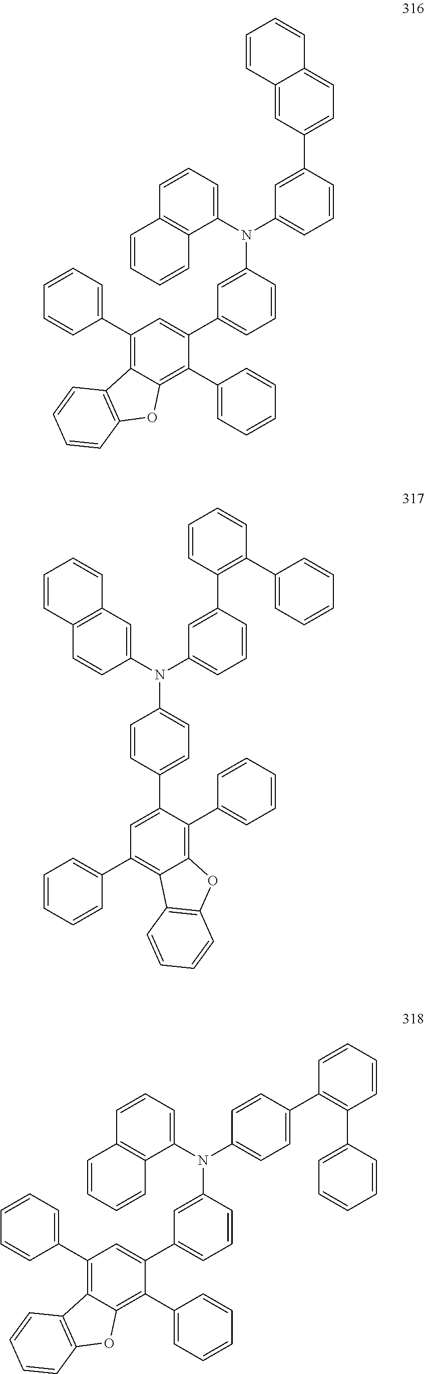 [SMILES: c1ccc(-c2c(-c3cccc(N(c4cccc(-c5ccc6ccccc6c5)c4)c4cccc5ccccc45)c3)cc(-c3ccccc3)c3c2oc2ccccc23)cc1 ; c1ccc(-c2ccccc2-c2cccc(N(c3ccc(-c4cc(-c5ccccc5)c5c(oc6ccccc65)c4-c4ccccc4)cc3)c3ccc4ccccc4c3)c2)cc1 ; c1ccc(-c2ccccc2-c2ccc(N(c3cccc(-c4cc(-c5ccccc5)c5c(oc6ccccc65)c4-c4ccccc4)c3)c3cccc4ccccc34)cc2)cc1]

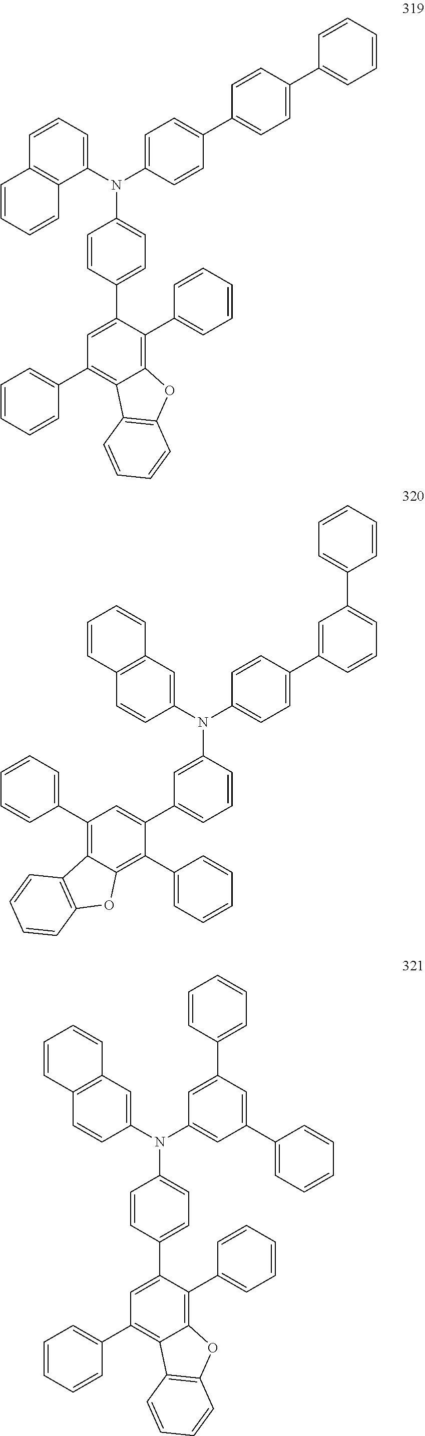 [SMILES: c1ccc(-c2ccc(-c3ccc(N(c4ccc(-c5cc(-c6ccccc6)c6c(oc7ccccc76)c5-c5ccccc5)cc4)c4cccc5ccccc45)cc3)cc2)cc1 ; c1ccc(-c2cccc(-c3ccc(N(c4cccc(-c5cc(-c6ccccc6)c6c(oc7ccccc76)c5-c5ccccc5)c4)c4ccc5ccccc5c4)cc3)c2)cc1 ; c1ccc(-c2cc(-c3ccccc3)cc(N(c3ccc(-c4cc(-c5ccccc5)c5c(oc6ccccc65)c4-c4ccccc4)cc3)c3ccc4ccccc4c3)c2)cc1]

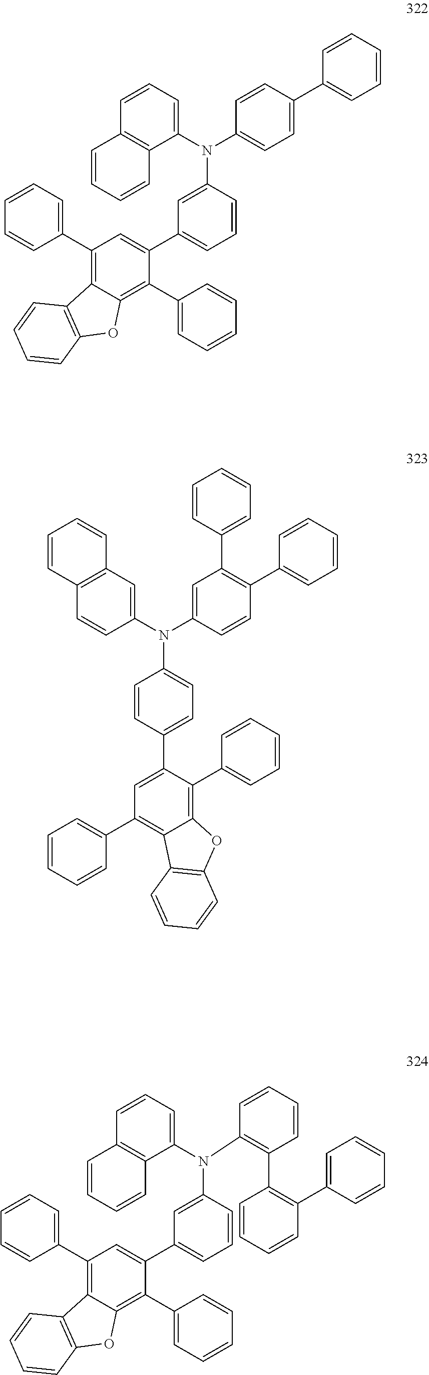 [SMILES: c1ccc(-c2ccc(N(c3cccc(-c4cc(-c5ccccc5)c5c(oc6ccccc65)c4-c4ccccc4)c3)c3cccc4ccccc34)cc2)cc1 ; c1ccc(-c2ccc(N(c3ccc(-c4cc(-c5ccccc5)c5c(oc6ccccc65)c4-c4ccccc4)cc3)c3ccc4ccccc4c3)cc2-c2ccccc2)cc1 ; c1ccc(-c2ccccc2-c2ccccc2N(c2cccc(-c3cc(-c4ccccc4)c4c(oc5ccccc54)c3-c3ccccc3)c2)c2cccc3ccccc23)cc1]

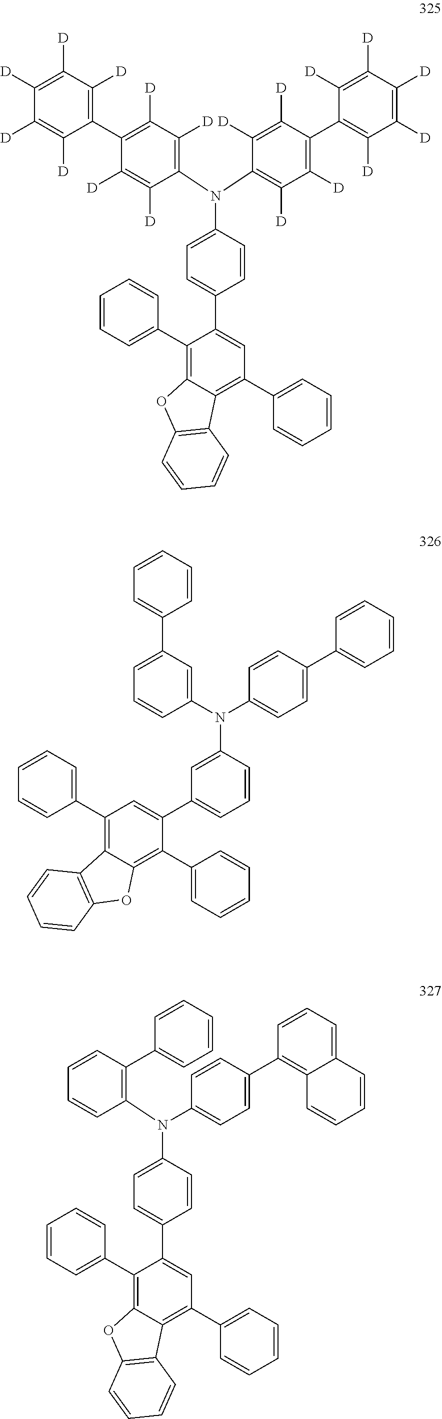 [SMILES: [2H]c1c([2H])c([2H])c(-c2c([2H])c([2H])c(N(c3ccc(-c4cc(-c5ccccc5)c5c(oc6ccccc65)c4-c4ccccc4)cc3)c3c([2H])c([2H])c(-c4c([2H])c([2H])c([2H])c([2H])c4[2H])c([2H])c3[2H])c([2H])c2[2H])c([2H])c1[2H] ; c1ccc(-c2ccc(N(c3cccc(-c4ccccc4)c3)c3cccc(-c4cc(-c5ccccc5)c5c(oc6ccccc65)c4-c4ccccc4)c3)cc2)cc1 ; c1ccc(-c2ccccc2N(c2ccc(-c3cc(-c4ccccc4)c4c(oc5ccccc54)c3-c3ccccc3)cc2)c2ccc(-c3cccc4ccccc34)cc2)cc1]

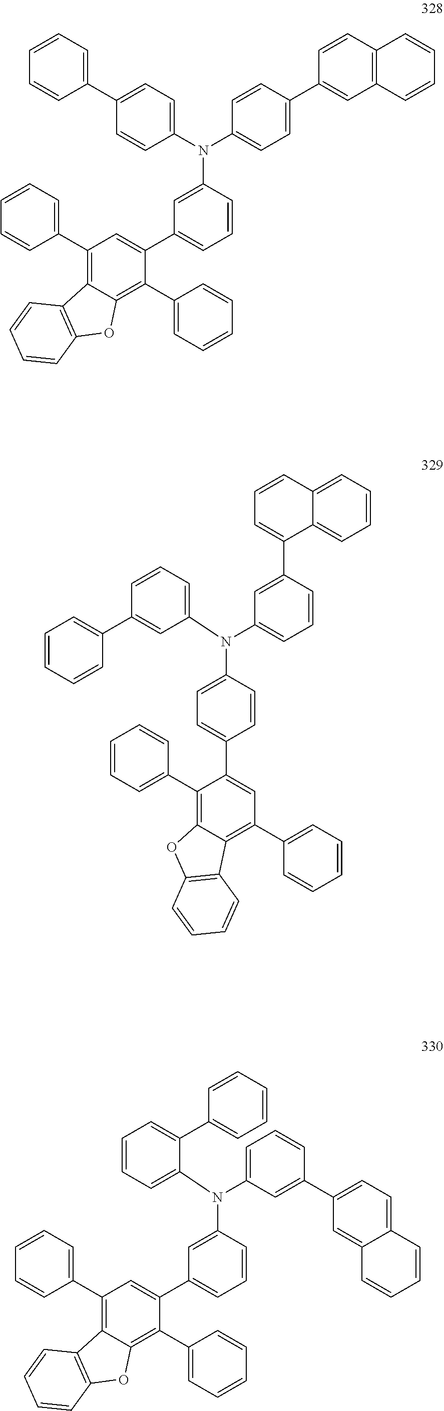 [SMILES: c1ccc(-c2ccc(N(c3ccc(-c4ccc5ccccc5c4)cc3)c3cccc(-c4cc(-c5ccccc5)c5c(oc6ccccc65)c4-c4ccccc4)c3)cc2)cc1 ; c1ccc(-c2cccc(N(c3ccc(-c4cc(-c5ccccc5)c5c(oc6ccccc65)c4-c4ccccc4)cc3)c3cccc(-c4cccc5ccccc45)c3)c2)cc1 ; c1ccc(-c2ccccc2N(c2cccc(-c3ccc4ccccc4c3)c2)c2cccc(-c3cc(-c4ccccc4)c4c(oc5ccccc54)c3-c3ccccc3)c2)cc1]

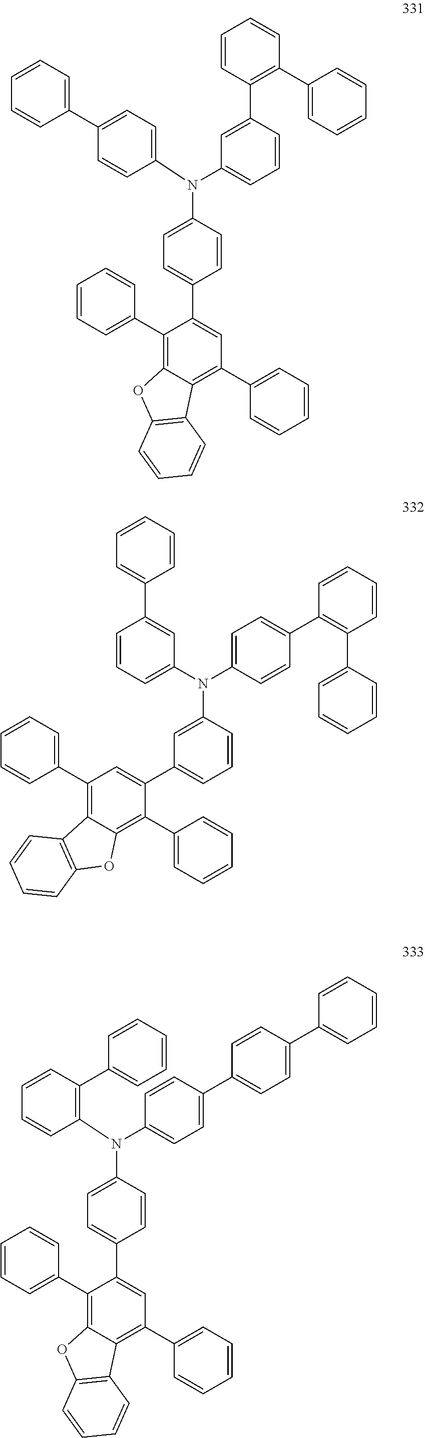

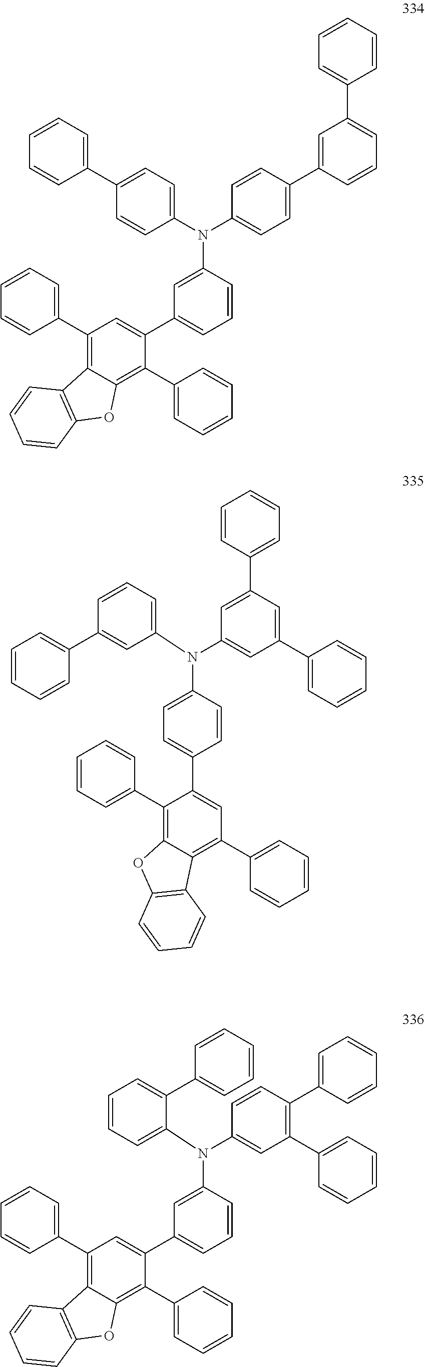 [SMILES: c1ccc(-c2ccc(N(c3ccc(-c4cccc(-c5ccccc5)c4)cc3)c3cccc(-c4cc(-c5ccccc5)c5c(oc6ccccc65)c4-c4ccccc4)c3)cc2)cc1 ; c1ccc(-c2cccc(N(c3ccc(-c4cc(-c5ccccc5)c5c(oc6ccccc65)c4-c4ccccc4)cc3)c3cc(-c4ccccc4)cc(-c4ccccc4)c3)c2)cc1 ; c1ccc(-c2ccc(N(c3cccc(-c4cc(-c5ccccc5)c5c(oc6ccccc65)c4-c4ccccc4)c3)c3ccccc3-c3ccccc3)cc2-c2ccccc2)cc1]

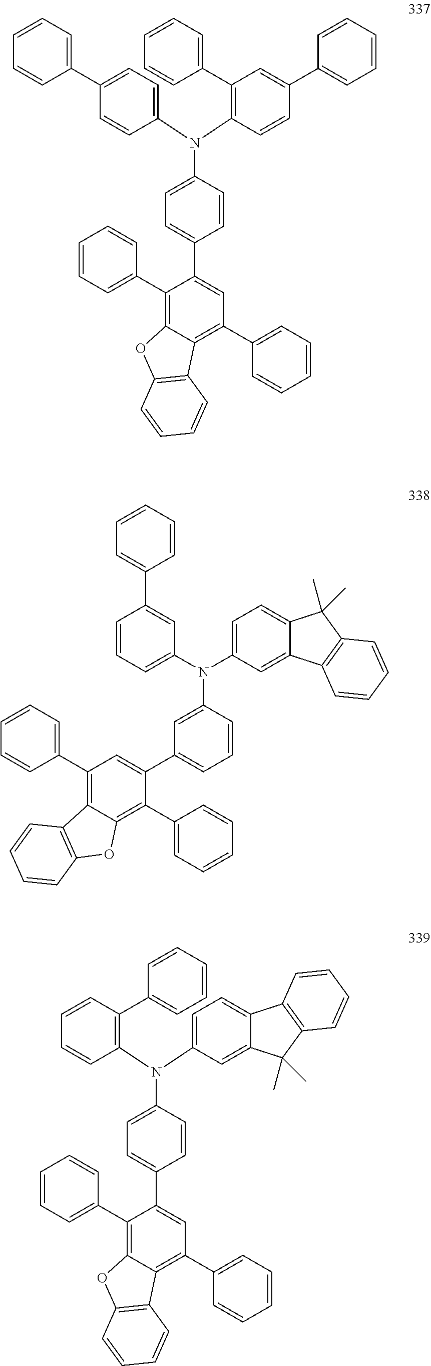 [SMILES: c1ccc(-c2ccc(N(c3ccc(-c4cc(-c5ccccc5)c5c(oc6ccccc65)c4-c4ccccc4)cc3)c3ccc(-c4ccccc4)cc3-c3ccccc3)cc2)cc1 ; CC1(C)c2ccccc2-c2cc(N(c3cccc(-c4ccccc4)c3)c3cccc(-c4cc(-c5ccccc5)c5c(oc6ccccc65)c4-c4ccccc4)c3)ccc21 ; CC1(C)c2ccccc2-c2ccc(N(c3ccc(-c4cc(-c5ccccc5)c5c(oc6ccccc65)c4-c4ccccc4)cc3)c3ccccc3-c3ccccc3)cc21]

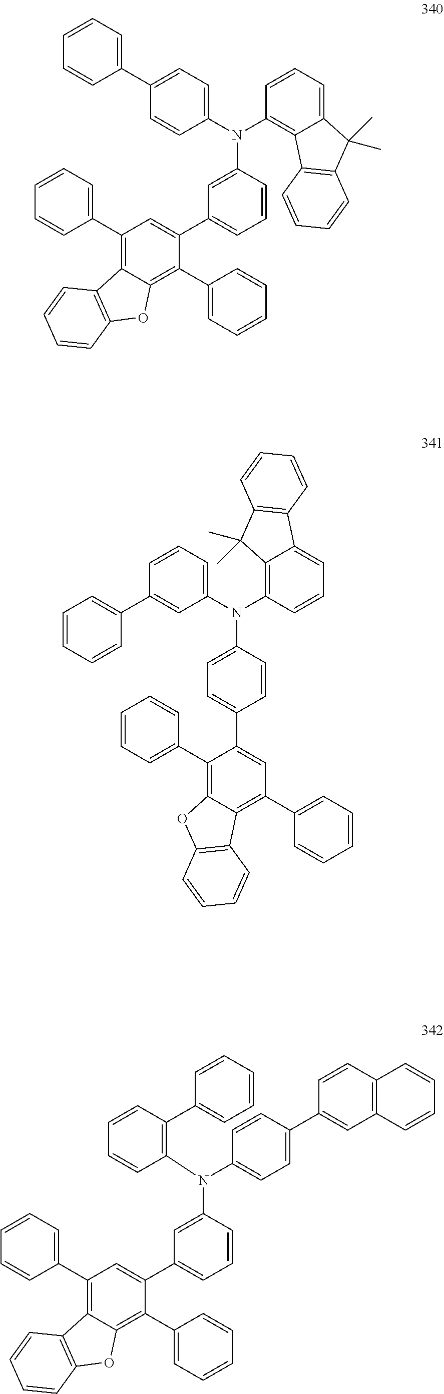 [SMILES: CC1(C)c2ccccc2-c2c(N(c3ccc(-c4ccccc4)cc3)c3cccc(-c4cc(-c5ccccc5)c5c(oc6ccccc65)c4-c4ccccc4)c3)cccc21 ; CC1(C)c2ccccc2-c2cccc(N(c3ccc(-c4cc(-c5ccccc5)c5c(oc6ccccc65)c4-c4ccccc4)cc3)c3cccc(-c4ccccc4)c3)c21 ; c1ccc(-c2ccccc2N(c2ccc(-c3ccc4ccccc4c3)cc2)c2cccc(-c3cc(-c4ccccc4)c4c(oc5ccccc54)c3-c3ccccc3)c2)cc1]

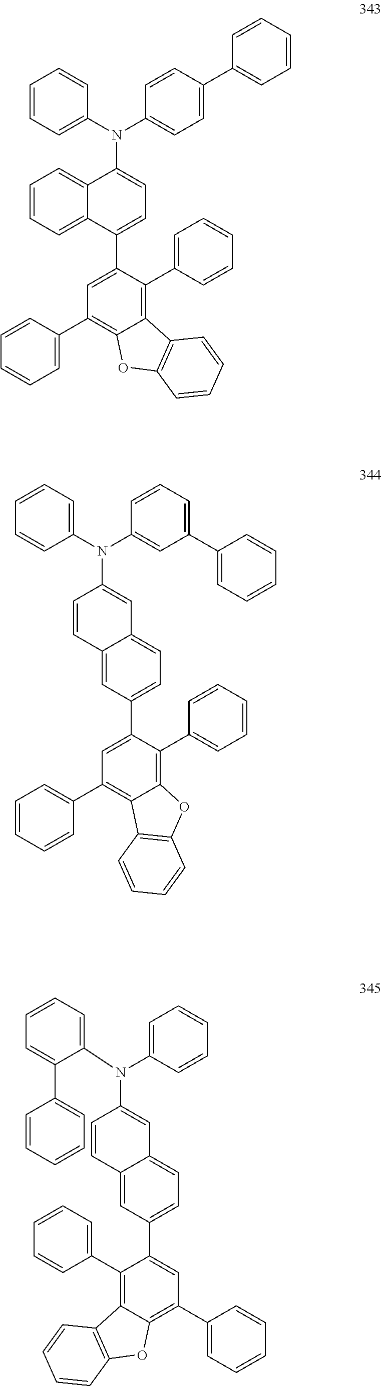 [SMILES: c1ccc(-c2ccc(N(c3ccccc3)c3ccc(-c4cc(-c5ccccc5)c5oc6ccccc6c5c4-c4ccccc4)c4ccccc34)cc2)cc1 ; c1ccc(-c2cccc(N(c3ccccc3)c3ccc4cc(-c5cc(-c6ccccc6)c6c(oc7ccccc76)c5-c5ccccc5)ccc4c3)c2)cc1 ; c1ccc(-c2ccccc2N(c2ccccc2)c2ccc3cc(-c4cc(-c5ccccc5)c5oc6ccccc6c5c4-c4ccccc4)ccc3c2)cc1]

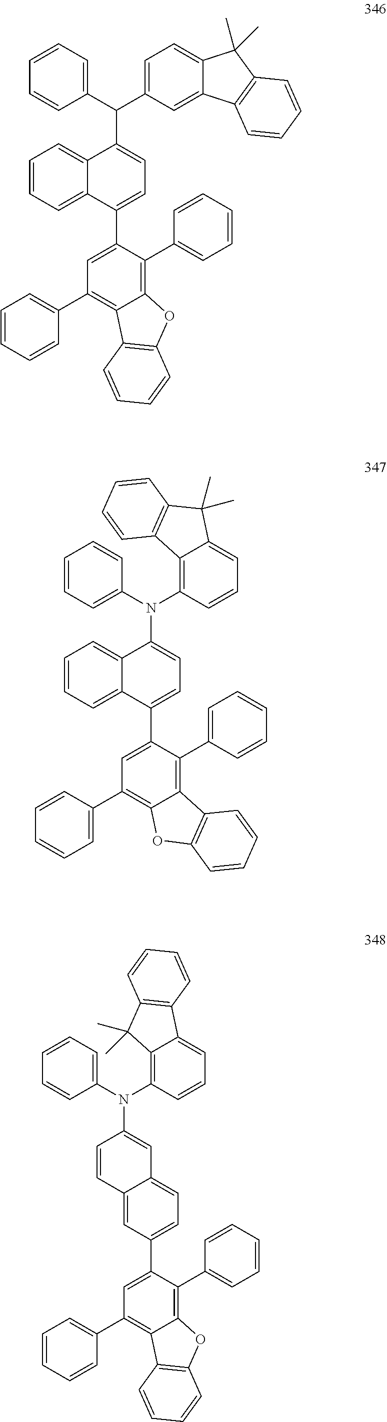 [SMILES: CC1(C)c2ccccc2-c2cc(C(c3ccccc3)c3ccc(-c4cc(-c5ccccc5)c5c(oc6ccccc65)c4-c4ccccc4)c4ccccc34)ccc21 ; CC1(C)c2ccccc2-c2c(N(c3ccccc3)c3ccc(-c4cc(-c5ccccc5)c5oc6ccccc6c5c4-c4ccccc4)c4ccccc34)cccc21 ; CC1(C)c2ccccc2-c2cccc(N(c3ccccc3)c3ccc4cc(-c5cc(-c6ccccc6)c6c(oc7ccccc76)c5-c5ccccc5)ccc4c3)c21]

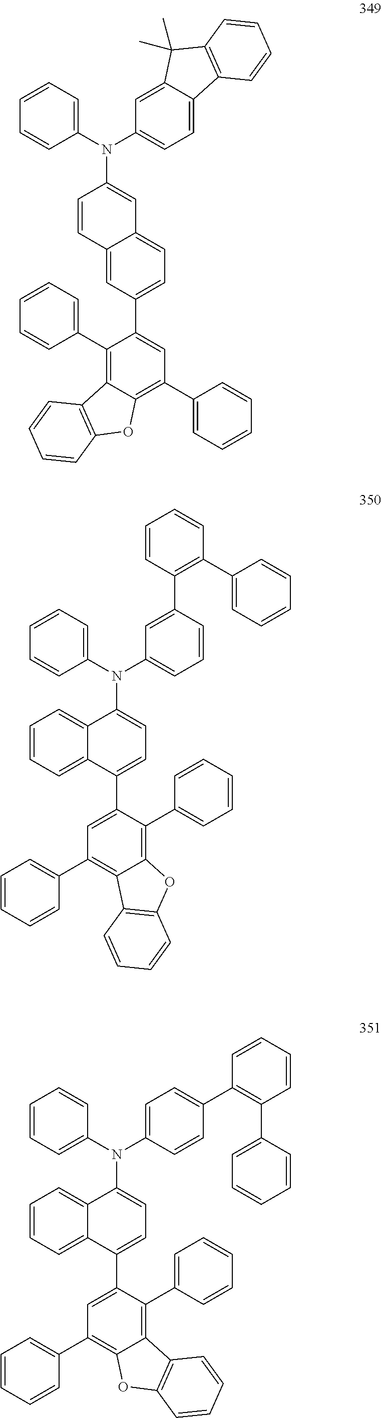 [SMILES: CC1(C)c2ccccc2-c2ccc(N(c3ccccc3)c3ccc4cc(-c5cc(-c6ccccc6)c6oc7ccccc7c6c5-c5ccccc5)ccc4c3)cc21 ; c1ccc(-c2ccccc2-c2cccc(N(c3ccccc3)c3ccc(-c4cc(-c5ccccc5)c5c(oc6ccccc65)c4-c4ccccc4)c4ccccc34)c2)cc1 ; c1ccc(-c2ccccc2-c2ccc(N(c3ccccc3)c3ccc(-c4cc(-c5ccccc5)c5oc6ccccc6c5c4-c4ccccc4)c4ccccc34)cc2)cc1]

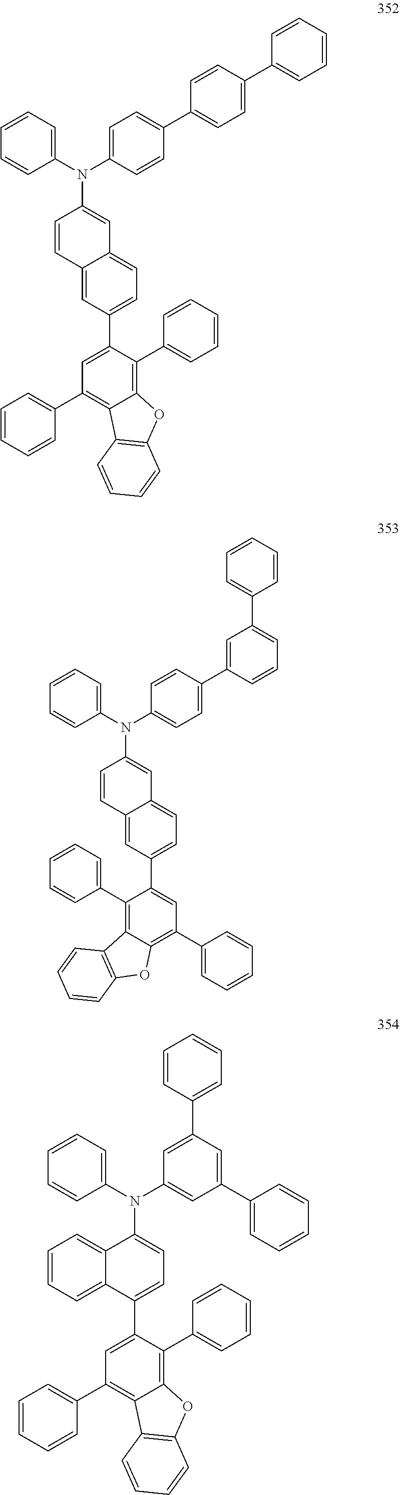 [SMILES: c1ccc(-c2ccc(-c3ccc(N(c4ccccc4)c4ccc5cc(-c6cc(-c7ccccc7)c7c(oc8ccccc87)c6-c6ccccc6)ccc5c4)cc3)cc2)cc1 ; c1ccc(-c2cccc(-c3ccc(N(c4ccccc4)c4ccc5cc(-c6cc(-c7ccccc7)c7oc8ccccc8c7c6-c6ccccc6)ccc5c4)cc3)c2)cc1 ; c1ccc(-c2cc(-c3ccccc3)cc(N(c3ccccc3)c3ccc(-c4cc(-c5ccccc5)c5c(oc6ccccc65)c4-c4ccccc4)c4ccccc34)c2)cc1]

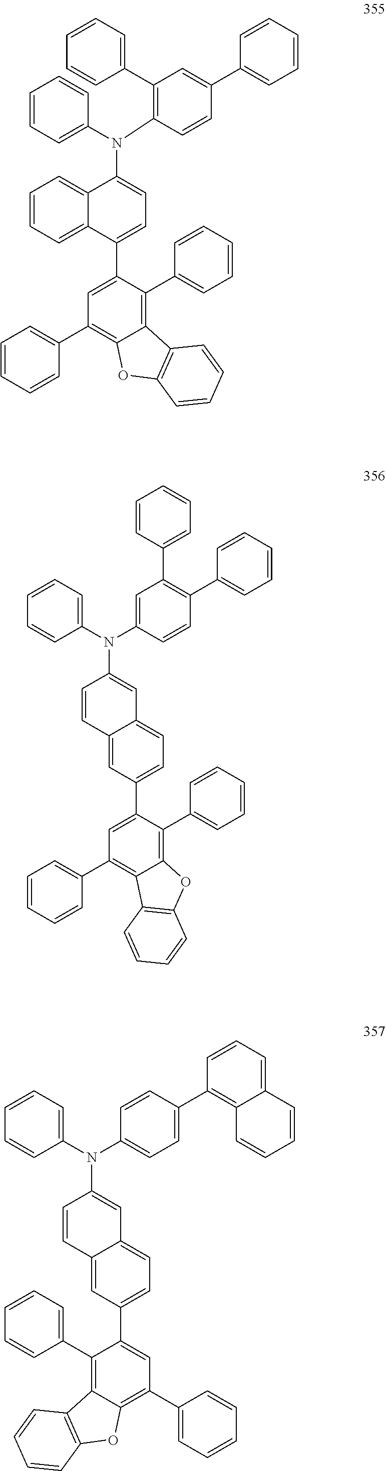 [SMILES: c1ccc(-c2ccc(N(c3ccccc3)c3ccc(-c4cc(-c5ccccc5)c5oc6ccccc6c5c4-c4ccccc4)c4ccccc34)c(-c3ccccc3)c2)cc1 ; c1ccc(-c2ccc(N(c3ccccc3)c3ccc4cc(-c5cc(-c6ccccc6)c6c(oc7ccccc76)c5-c5ccccc5)ccc4c3)cc2-c2ccccc2)cc1 ; c1ccc(-c2cc(-c3ccc4cc(N(c5ccccc5)c5ccc(-c6cccc7ccccc67)cc5)ccc4c3)c(-c3ccccc3)c3c2oc2ccccc23)cc1]

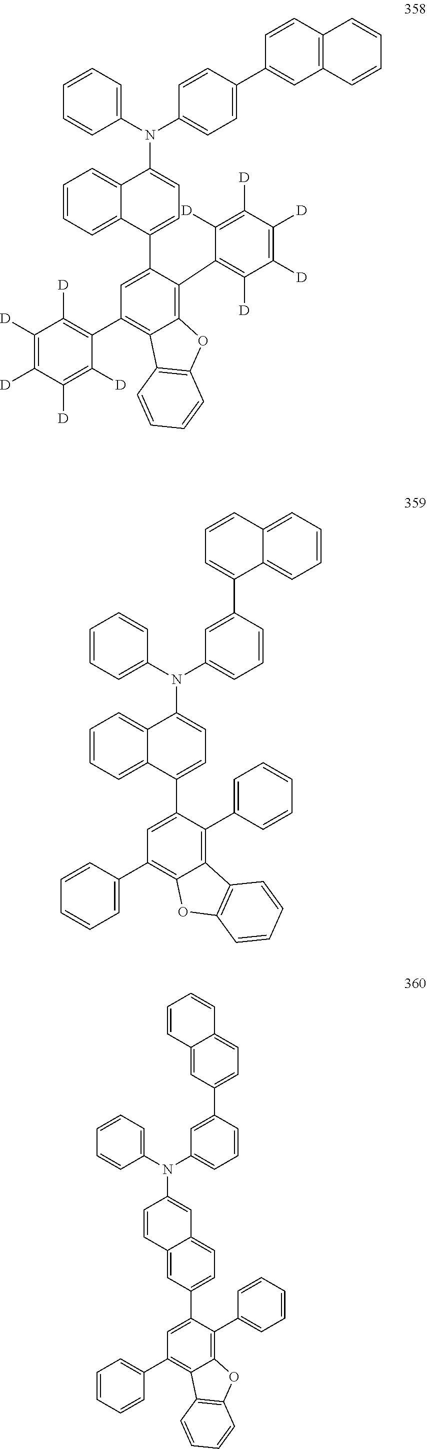 [SMILES: [2H]c1c([2H])c([2H])c(-c2c(-c3ccc(N(c4ccccc4)c4ccc(-c5ccc6ccccc6c5)cc4)c4ccccc34)cc(-c3c([2H])c([2H])c([2H])c([2H])c3[2H])c3c2oc2ccccc23)c([2H])c1[2H] ; c1ccc(-c2cc(-c3ccc(N(c4ccccc4)c4cccc(-c5cccc6ccccc56)c4)c4ccccc34)c(-c3ccccc3)c3c2oc2ccccc23)cc1 ; c1ccc(-c2c(-c3ccc4cc(N(c5ccccc5)c5cccc(-c6ccc7ccccc7c6)c5)ccc4c3)cc(-c3ccccc3)c3c2oc2ccccc23)cc1]

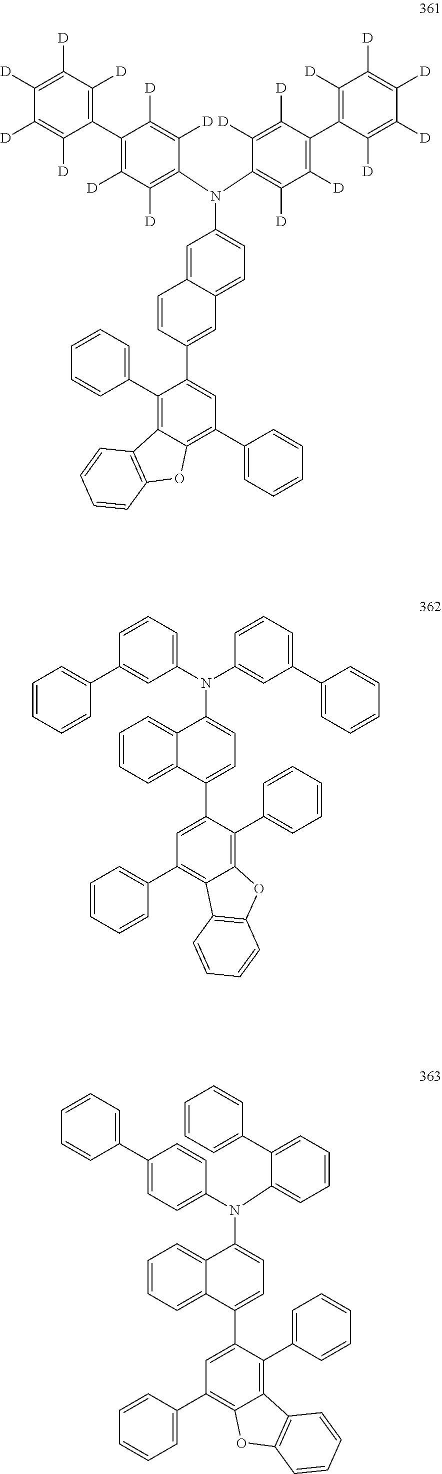 [SMILES: [2H]c1c([2H])c([2H])c(-c2c([2H])c([2H])c(N(c3ccc4cc(-c5cc(-c6ccccc6)c6oc7ccccc7c6c5-c5ccccc5)ccc4c3)c3c([2H])c([2H])c(-c4c([2H])c([2H])c([2H])c([2H])c4[2H])c([2H])c3[2H])c([2H])c2[2H])c([2H])c1[2H] ; c1ccc(-c2cccc(N(c3cccc(-c4ccccc4)c3)c3ccc(-c4cc(-c5ccccc5)c5c(oc6ccccc65)c4-c4ccccc4)c4ccccc34)c2)cc1 ; c1ccc(-c2ccc(N(c3ccccc3-c3ccccc3)c3ccc(-c4cc(-c5ccccc5)c5oc6ccccc6c5c4-c4ccccc4)c4ccccc34)cc2)cc1]

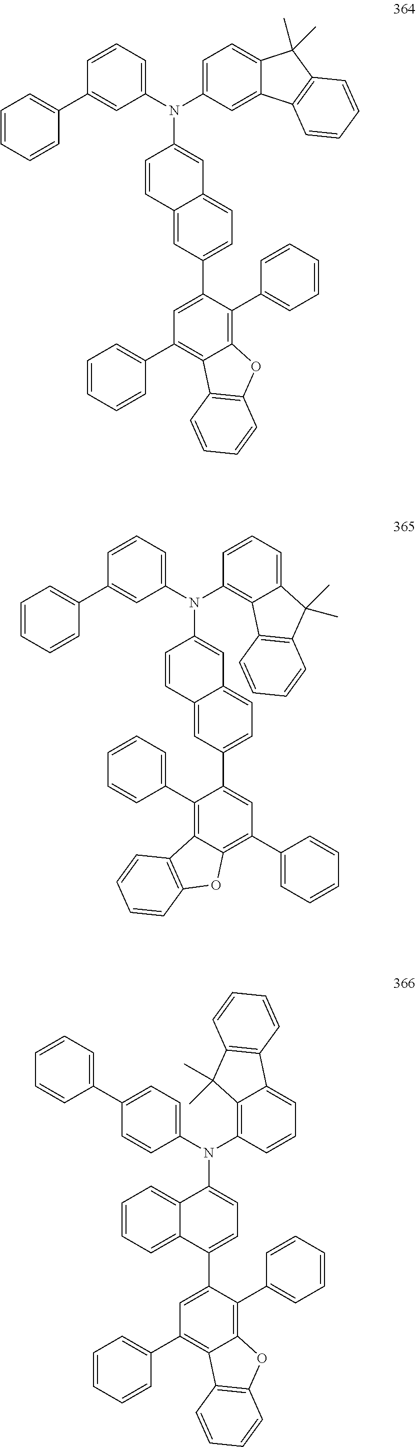 [SMILES: CC1(C)c2ccccc2-c2cc(N(c3cccc(-c4ccccc4)c3)c3ccc4cc(-c5cc(-c6ccccc6)c6c(oc7ccccc76)c5-c5ccccc5)ccc4c3)ccc21 ; CC1(C)c2ccccc2-c2c(N(c3cccc(-c4ccccc4)c3)c3ccc4cc(-c5cc(-c6ccccc6)c6oc7ccccc7c6c5-c5ccccc5)ccc4c3)cccc21 ; CC1(C)c2ccccc2-c2cccc(N(c3ccc(-c4ccccc4)cc3)c3ccc(-c4cc(-c5ccccc5)c5c(oc6ccccc65)c4-c4ccccc4)c4ccccc34)c21]

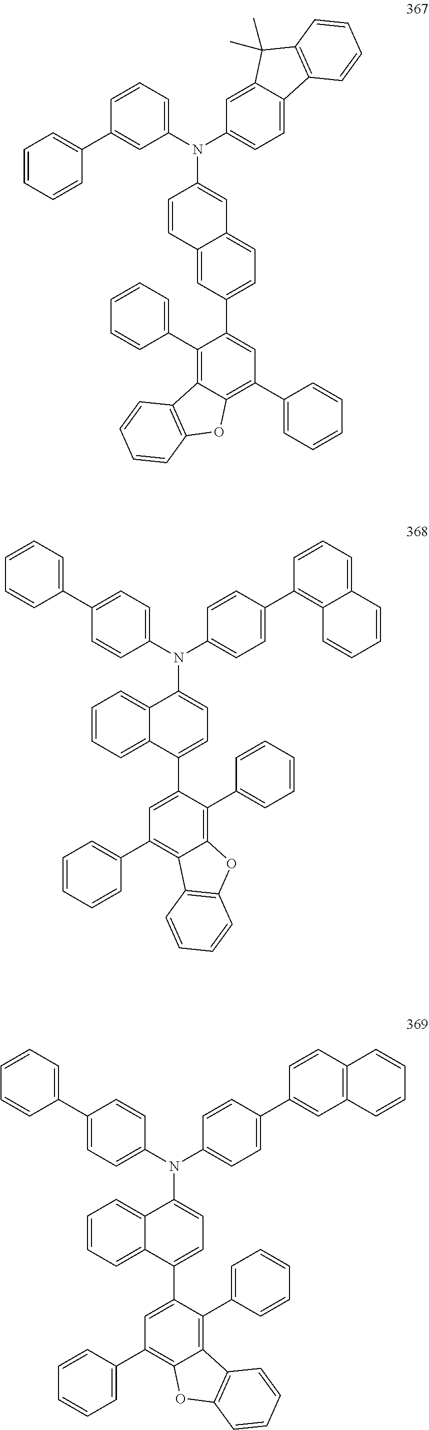 [SMILES: CC1(C)c2ccccc2-c2ccc(N(c3cccc(-c4ccccc4)c3)c3ccc4cc(-c5cc(-c6ccccc6)c6oc7ccccc7c6c5-c5ccccc5)ccc4c3)cc21 ; c1ccc(-c2ccc(N(c3ccc(-c4cccc5ccccc45)cc3)c3ccc(-c4cc(-c5ccccc5)c5c(oc6ccccc65)c4-c4ccccc4)c4ccccc34)cc2)cc1 ; c1ccc(-c2ccc(N(c3ccc(-c4ccc5ccccc5c4)cc3)c3ccc(-c4cc(-c5ccccc5)c5oc6ccccc6c5c4-c4ccccc4)c4ccccc34)cc2)cc1]

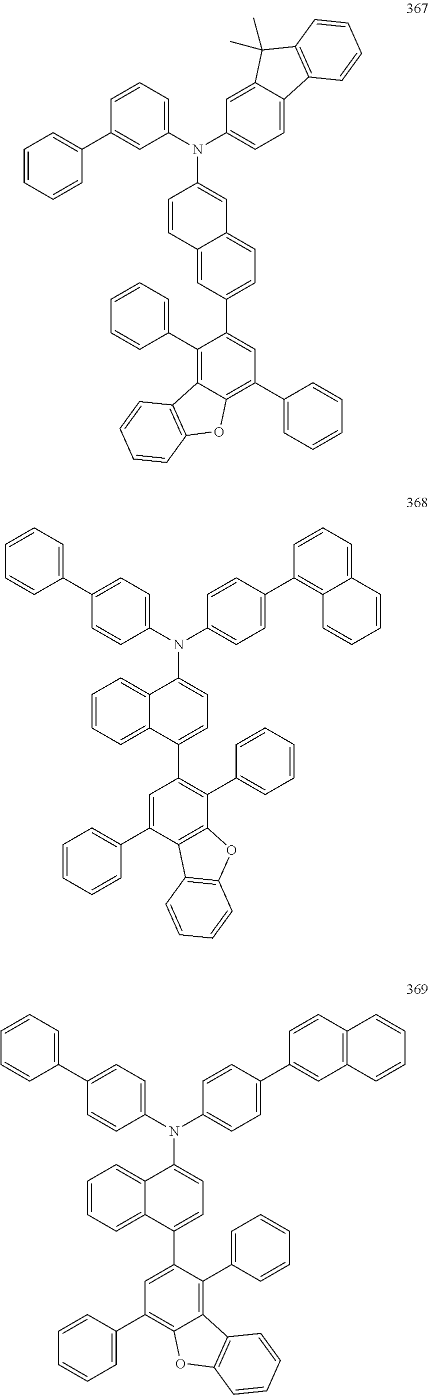 [SMILES: CC1(C)c2ccccc2-c2ccc(N(c3cccc(-c4ccccc4)c3)c3ccc4cc(-c5cc(-c6ccccc6)c6oc7ccccc7c6c5-c5ccccc5)ccc4c3)cc21 ; c1ccc(-c2ccc(N(c3ccc(-c4cccc5ccccc45)cc3)c3ccc(-c4cc(-c5ccccc5)c5c(oc6ccccc65)c4-c4ccccc4)c4ccccc34)cc2)cc1 ; c1ccc(-c2ccc(N(c3ccc(-c4ccc5ccccc5c4)cc3)c3ccc(-c4cc(-c5ccccc5)c5oc6ccccc6c5c4-c4ccccc4)c4ccccc34)cc2)cc1]

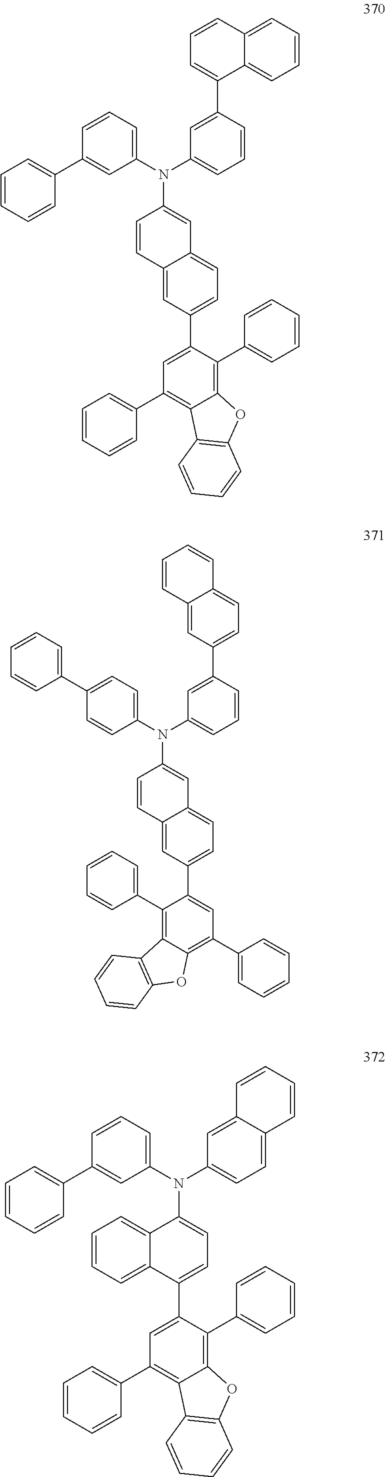 [SMILES: c1ccc(-c2cccc(N(c3cccc(-c4cccc5ccccc45)c3)c3ccc4cc(-c5cc(-c6ccccc6)c6c(oc7ccccc76)c5-c5ccccc5)ccc4c3)c2)cc1 ; c1ccc(-c2ccc(N(c3cccc(-c4ccc5ccccc5c4)c3)c3ccc4cc(-c5cc(-c6ccccc6)c6oc7ccccc7c6c5-c5ccccc5)ccc4c3)cc2)cc1 ; c1ccc(-c2cccc(N(c3ccc4ccccc4c3)c3ccc(-c4cc(-c5ccccc5)c5c(oc6ccccc65)c4-c4ccccc4)c4ccccc34)c2)cc1]

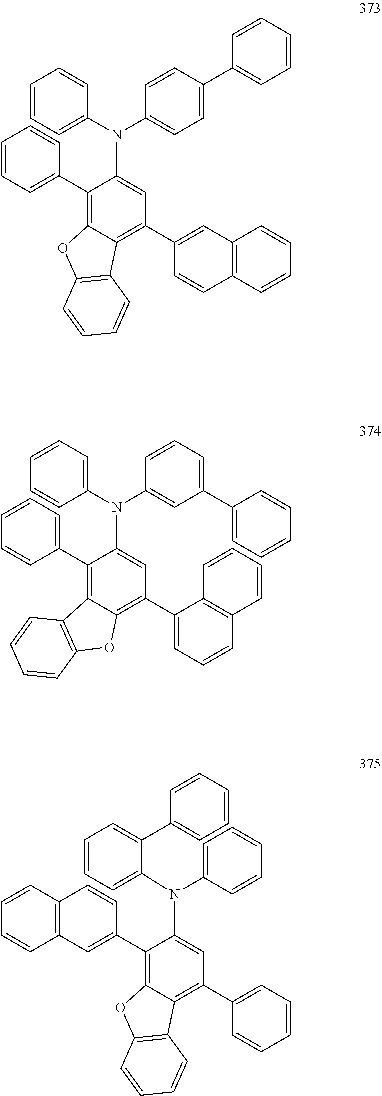 [SMILES: c1ccc(-c2ccc(N(c3ccccc3)c3cc(-c4ccc5ccccc5c4)c4c(oc5ccccc54)c3-c3ccccc3)cc2)cc1 ; c1ccc(-c2cccc(N(c3ccccc3)c3cc(-c4cccc5ccccc45)c4oc5ccccc5c4c3-c3ccccc3)c2)cc1 ; c1ccc(-c2ccccc2N(c2ccccc2)c2cc(-c3ccccc3)c3c(oc4ccccc43)c2-c2ccc3ccccc3c2)cc1]

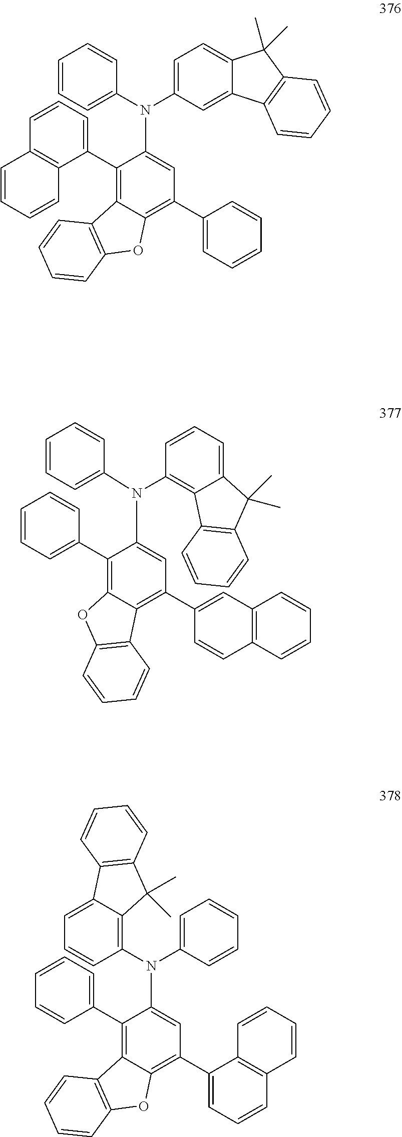 [SMILES: CC1(C)c2ccccc2-c2cc(N(c3ccccc3)c3cc(-c4ccccc4)c4oc5ccccc5c4c3-c3cccc4ccccc34)ccc21 ; CC1(C)c2ccccc2-c2c(N(c3ccccc3)c3cc(-c4ccc5ccccc5c4)c4c(oc5ccccc54)c3-c3ccccc3)cccc21 ; CC1(C)c2ccccc2-c2cccc(N(c3ccccc3)c3cc(-c4cccc5ccccc45)c4oc5ccccc5c4c3-c3ccccc3)c21]

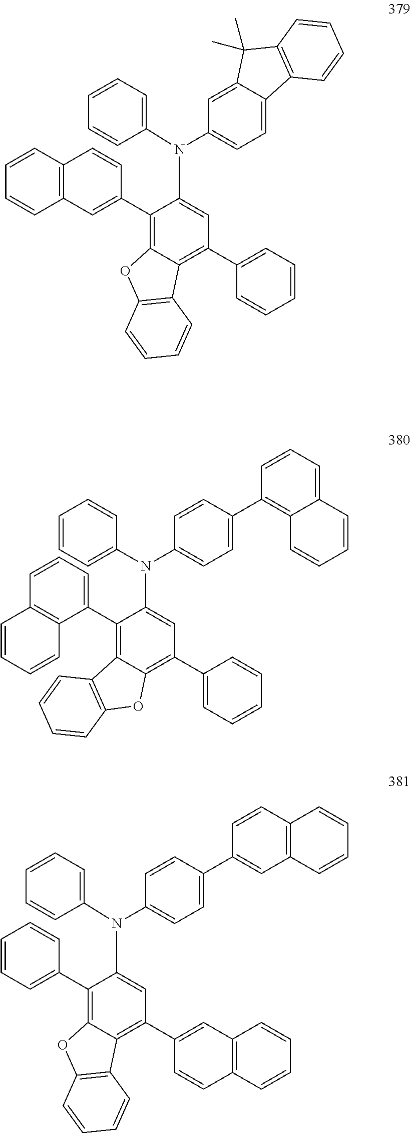 [SMILES: CC1(C)c2ccccc2-c2ccc(N(c3ccccc3)c3cc(-c4ccccc4)c4c(oc5ccccc54)c3-c3ccc4ccccc4c3)cc21 ; c1ccc(-c2cc(N(c3ccccc3)c3ccc(-c4cccc5ccccc45)cc3)c(-c3cccc4ccccc34)c3c2oc2ccccc23)cc1 ; c1ccc(-c2c(N(c3ccccc3)c3ccc(-c4ccc5ccccc5c4)cc3)cc(-c3ccc4ccccc4c3)c3c2oc2ccccc23)cc1]

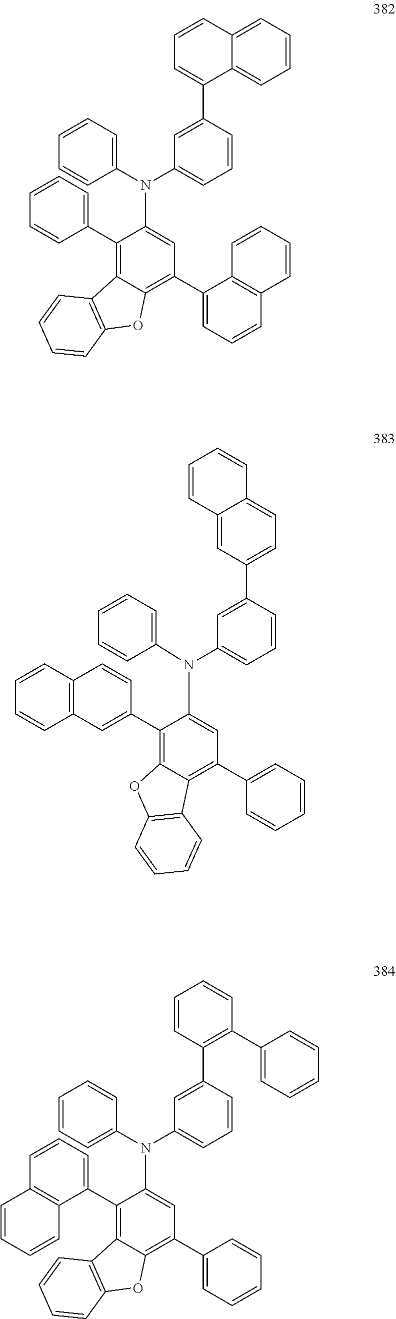 [SMILES: c1ccc(-c2c(N(c3ccccc3)c3cccc(-c4cccc5ccccc45)c3)cc(-c3cccc4ccccc34)c3oc4ccccc4c23)cc1 ; c1ccc(-c2cc(N(c3ccccc3)c3cccc(-c4ccc5ccccc5c4)c3)c(-c3ccc4ccccc4c3)c3oc4ccccc4c23)cc1 ; c1ccc(-c2ccccc2-c2cccc(N(c3ccccc3)c3cc(-c4ccccc4)c4oc5ccccc5c4c3-c3cccc4ccccc34)c2)cc1]

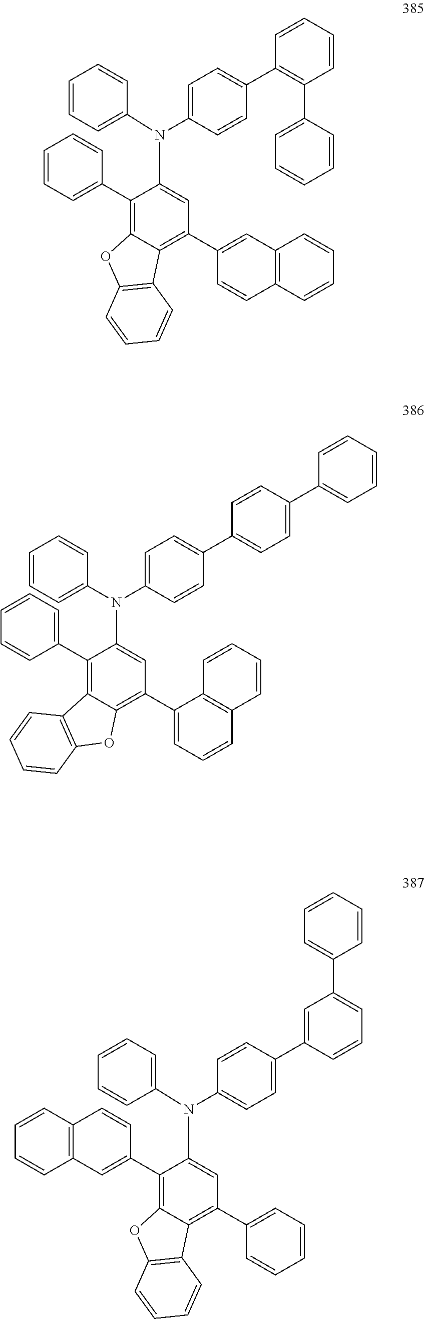 [SMILES: c1ccc(-c2ccccc2-c2ccc(N(c3ccccc3)c3cc(-c4ccc5ccccc5c4)c4c(oc5ccccc54)c3-c3ccccc3)cc2)cc1 ; c1ccc(-c2ccc(-c3ccc(N(c4ccccc4)c4cc(-c5cccc6ccccc56)c5oc6ccccc6c5c4-c4ccccc4)cc3)cc2)cc1 ; c1ccc(-c2cccc(-c3ccc(N(c4ccccc4)c4cc(-c5ccccc5)c5c(oc6ccccc65)c4-c4ccc5ccccc5c4)cc3)c2)cc1]

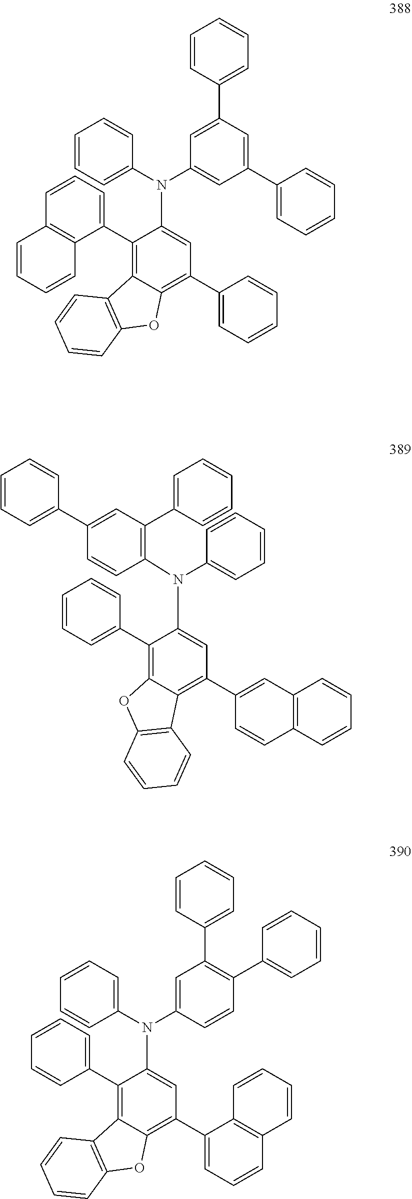 [SMILES: c1ccc(-c2cc(-c3ccccc3)cc(N(c3ccccc3)c3cc(-c4ccccc4)c4oc5ccccc5c4c3-c3cccc4ccccc34)c2)cc1 ; c1ccc(-c2ccc(N(c3ccccc3)c3cc(-c4ccc5ccccc5c4)c4c(oc5ccccc54)c3-c3ccccc3)c(-c3ccccc3)c2)cc1 ; c1ccc(-c2ccc(N(c3ccccc3)c3cc(-c4cccc5ccccc45)c4oc5ccccc5c4c3-c3ccccc3)cc2-c2ccccc2)cc1]

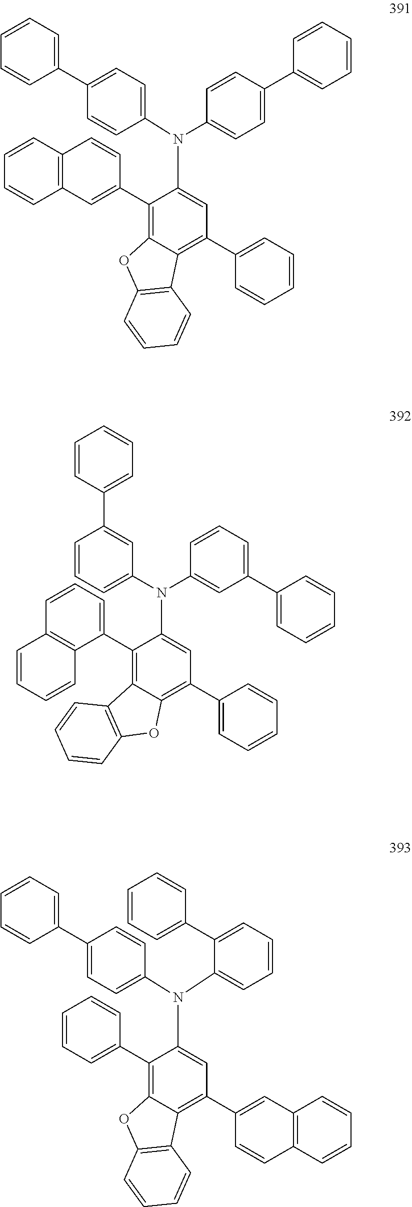 [SMILES: c1ccc(-c2ccc(N(c3ccc(-c4ccccc4)cc3)c3cc(-c4ccccc4)c4c(oc5ccccc54)c3-c3ccc4ccccc4c3)cc2)cc1 ; c1ccc(-c2cccc(N(c3cccc(-c4ccccc4)c3)c3cc(-c4ccccc4)c4oc5ccccc5c4c3-c3cccc4ccccc34)c2)cc1 ; c1ccc(-c2ccc(N(c3ccccc3-c3ccccc3)c3cc(-c4ccc5ccccc5c4)c4c(oc5ccccc54)c3-c3ccccc3)cc2)cc1]

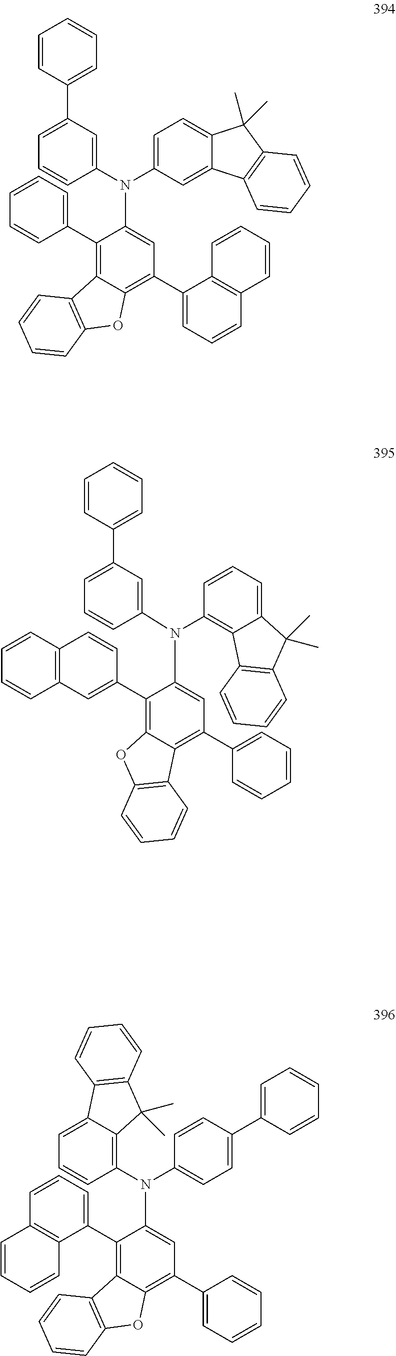 [SMILES: CC1(C)c2ccccc2-c2cc(N(c3cccc(-c4ccccc4)c3)c3cc(-c4cccc5ccccc45)c4oc5ccccc5c4c3-c3ccccc3)ccc21 ; CC1(C)c2ccccc2-c2c(N(c3cccc(-c4ccccc4)c3)c3cc(-c4ccccc4)c4c(oc5ccccc54)c3-c3ccc4ccccc4c3)cccc21 ; CC1(C)c2ccccc2-c2cccc(N(c3ccc(-c4ccccc4)cc3)c3cc(-c4ccccc4)c4oc5ccccc5c4c3-c3cccc4ccccc34)c21]

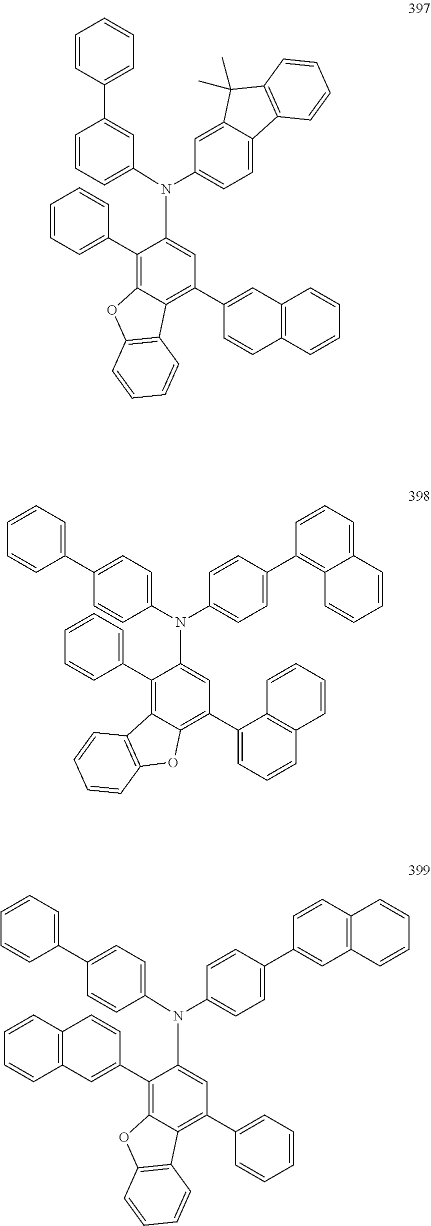 [SMILES: CC1(C)c2ccccc2-c2ccc(N(c3cccc(-c4ccccc4)c3)c3cc(-c4ccc5ccccc5c4)c4c(oc5ccccc54)c3-c3ccccc3)cc21 ; c1ccc(-c2ccc(N(c3ccc(-c4cccc5ccccc45)cc3)c3cc(-c4cccc5ccccc45)c4oc5ccccc5c4c3-c3ccccc3)cc2)cc1 ; c1ccc(-c2ccc(N(c3ccc(-c4ccc5ccccc5c4)cc3)c3cc(-c4ccccc4)c4c(oc5ccccc54)c3-c3ccc4ccccc4c3)cc2)cc1]

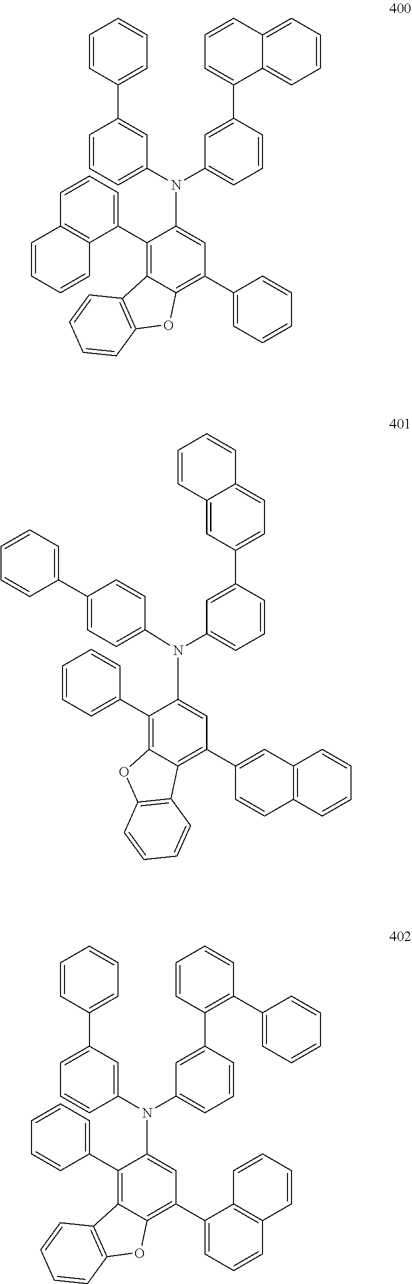 [SMILES: c1ccc(-c2cccc(N(c3cccc(-c4cccc5ccccc45)c3)c3cc(-c4ccccc4)c4oc5ccccc5c4c3-c3cccc4ccccc34)c2)cc1 ; c1ccc(-c2ccc(N(c3cccc(-c4ccc5ccccc5c4)c3)c3cc(-c4ccc5ccccc5c4)c4c(oc5ccccc54)c3-c3ccccc3)cc2)cc1 ; c1ccc(-c2cccc(N(c3cccc(-c4ccccc4-c4ccccc4)c3)c3cc(-c4cccc5ccccc45)c4oc5ccccc5c4c3-c3ccccc3)c2)cc1]

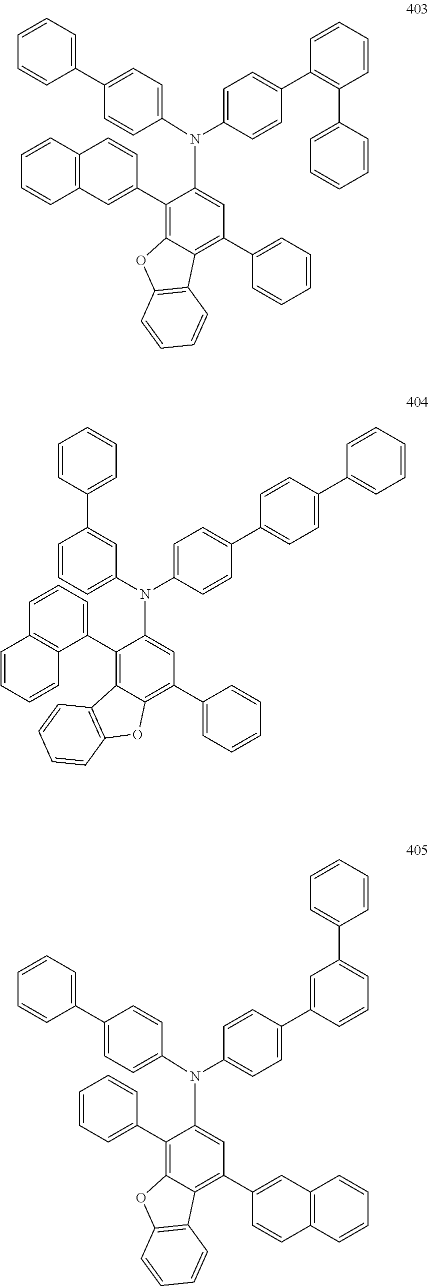 [SMILES: c1ccc(-c2ccc(N(c3ccc(-c4ccccc4-c4ccccc4)cc3)c3cc(-c4ccccc4)c4c(oc5ccccc54)c3-c3ccc4ccccc4c3)cc2)cc1 ; c1ccc(-c2ccc(-c3ccc(N(c4cccc(-c5ccccc5)c4)c4cc(-c5ccccc5)c5oc6ccccc6c5c4-c4cccc5ccccc45)cc3)cc2)cc1 ; c1ccc(-c2ccc(N(c3ccc(-c4cccc(-c5ccccc5)c4)cc3)c3cc(-c4ccc5ccccc5c4)c4c(oc5ccccc54)c3-c3ccccc3)cc2)cc1]

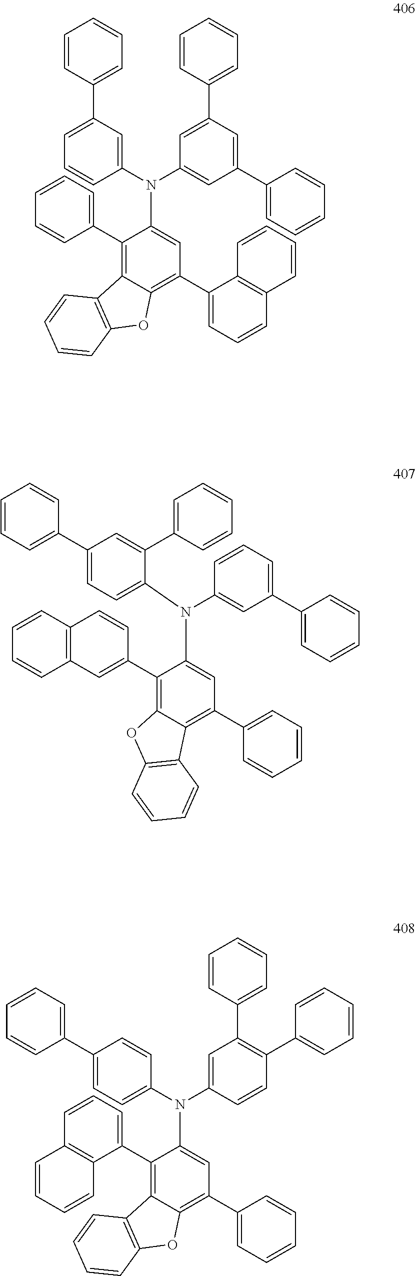 [SMILES: c1ccc(-c2cccc(N(c3cc(-c4ccccc4)cc(-c4ccccc4)c3)c3cc(-c4cccc5ccccc45)c4oc5ccccc5c4c3-c3ccccc3)c2)cc1 ; c1ccc(-c2cccc(N(c3ccc(-c4ccccc4)cc3-c3ccccc3)c3cc(-c4ccccc4)c4c(oc5ccccc54)c3-c3ccc4ccccc4c3)c2)cc1 ; c1ccc(-c2ccc(N(c3ccc(-c4ccccc4)c(-c4ccccc4)c3)c3cc(-c4ccccc4)c4oc5ccccc5c4c3-c3cccc4ccccc34)cc2)cc1]

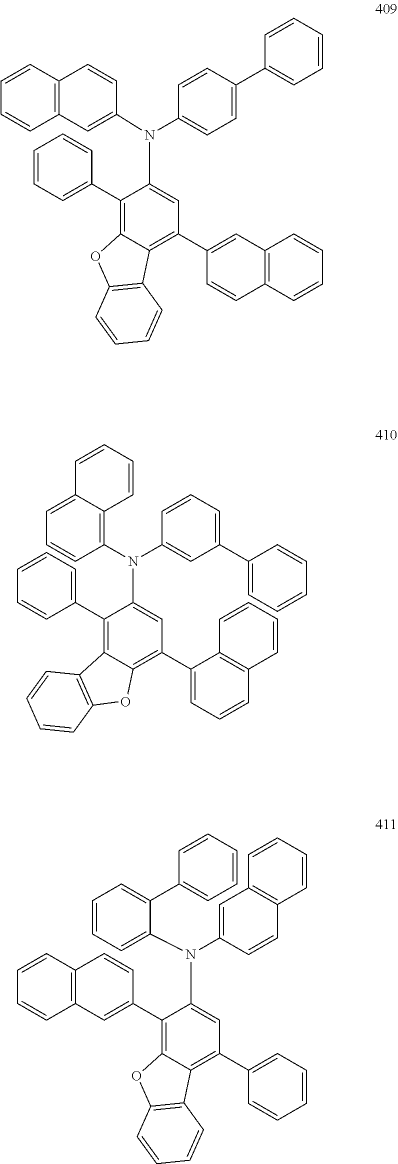 [SMILES: c1ccc(-c2ccc(N(c3ccc4ccccc4c3)c3cc(-c4ccc5ccccc5c4)c4c(oc5ccccc54)c3-c3ccccc3)cc2)cc1 ; c1ccc(-c2cccc(N(c3cc(-c4cccc5ccccc45)c4oc5ccccc5c4c3-c3ccccc3)c3cccc4ccccc34)c2)cc1 ; c1ccc(-c2ccccc2N(c2ccc3ccccc3c2)c2cc(-c3ccccc3)c3c(oc4ccccc43)c2-c2ccc3ccccc3c2)cc1]

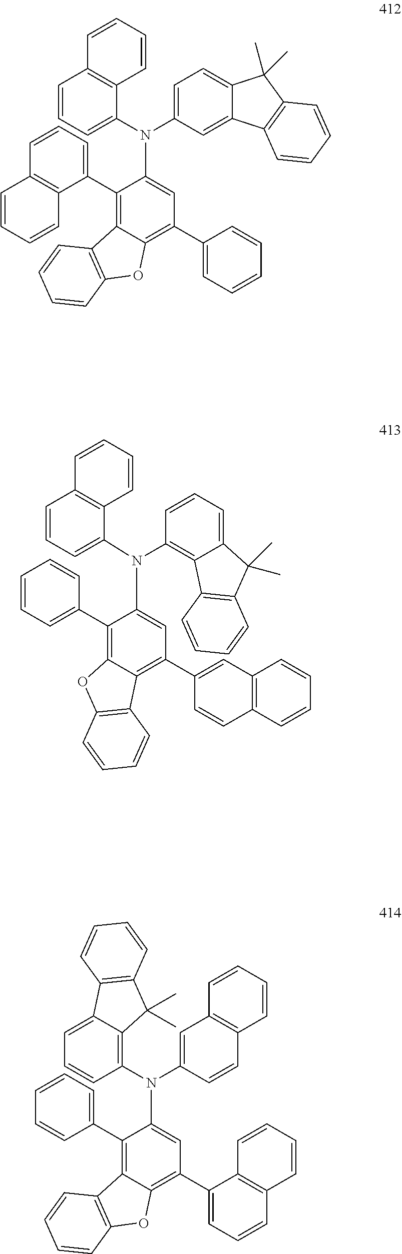 [SMILES: CC1(C)c2ccccc2-c2cc(N(c3cc(-c4ccccc4)c4oc5ccccc5c4c3-c3cccc4ccccc34)c3cccc4ccccc34)ccc21 ; CC1(C)c2ccccc2-c2c(N(c3cc(-c4ccc5ccccc5c4)c4c(oc5ccccc54)c3-c3ccccc3)c3cccc4ccccc34)cccc21 ; CC1(C)c2ccccc2-c2cccc(N(c3ccc4ccccc4c3)c3cc(-c4cccc5ccccc45)c4oc5ccccc5c4c3-c3ccccc3)c21]

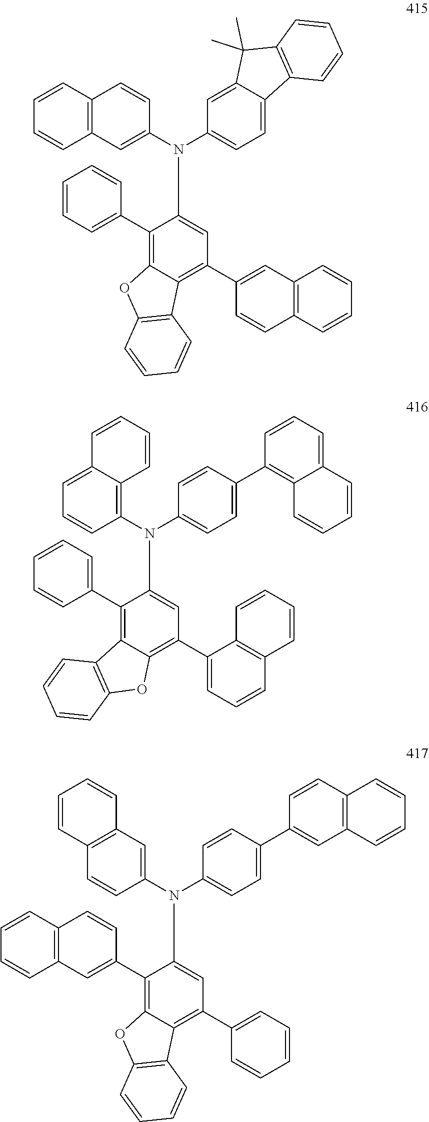 [SMILES: CC1(C)c2ccccc2-c2ccc(N(c3ccc4ccccc4c3)c3cc(-c4ccc5ccccc5c4)c4c(oc5ccccc54)c3-c3ccccc3)cc21 ; c1ccc(-c2c(N(c3ccc(-c4cccc5ccccc45)cc3)c3cccc4ccccc34)cc(-c3cccc4ccccc34)c3oc4ccccc4c23)cc1 ; c1ccc(-c2cc(N(c3ccc(-c4ccc5ccccc5c4)cc3)c3ccc4ccccc4c3)c(-c3ccc4ccccc4c3)c3oc4ccccc4c23)cc1]

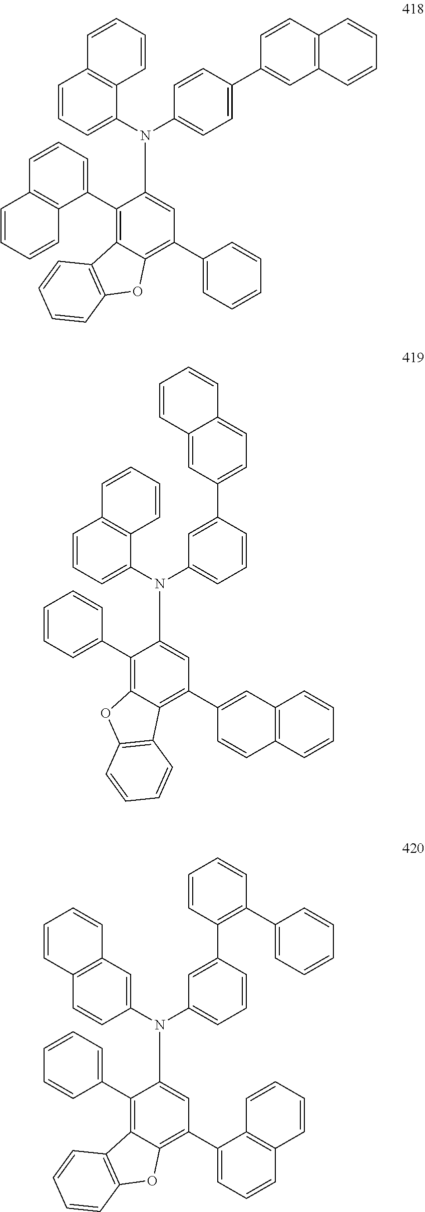 [SMILES: c1ccc(-c2cc(N(c3ccc(-c4ccc5ccccc5c4)cc3)c3cccc4ccccc34)c(-c3cccc4ccccc34)c3c2oc2ccccc23)cc1 ; c1ccc(-c2c(N(c3cccc(-c4ccc5ccccc5c4)c3)c3cccc4ccccc34)cc(-c3ccc4ccccc4c3)c3c2oc2ccccc23)cc1 ; c1ccc(-c2ccccc2-c2cccc(N(c3ccc4ccccc4c3)c3cc(-c4cccc5ccccc45)c4oc5ccccc5c4c3-c3ccccc3)c2)cc1]

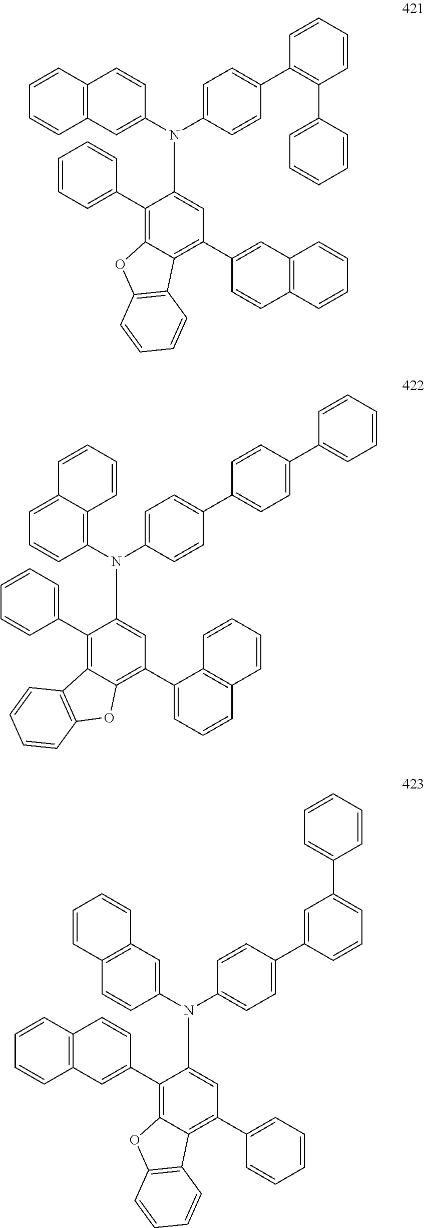 [SMILES: c1ccc(-c2ccccc2-c2ccc(N(c3ccc4ccccc4c3)c3cc(-c4ccc5ccccc5c4)c4c(oc5ccccc54)c3-c3ccccc3)cc2)cc1 ; c1ccc(-c2ccc(-c3ccc(N(c4cc(-c5cccc6ccccc56)c5oc6ccccc6c5c4-c4ccccc4)c4cccc5ccccc45)cc3)cc2)cc1 ; c1ccc(-c2cccc(-c3ccc(N(c4ccc5ccccc5c4)c4cc(-c5ccccc5)c5c(oc6ccccc65)c4-c4ccc5ccccc5c4)cc3)c2)cc1]

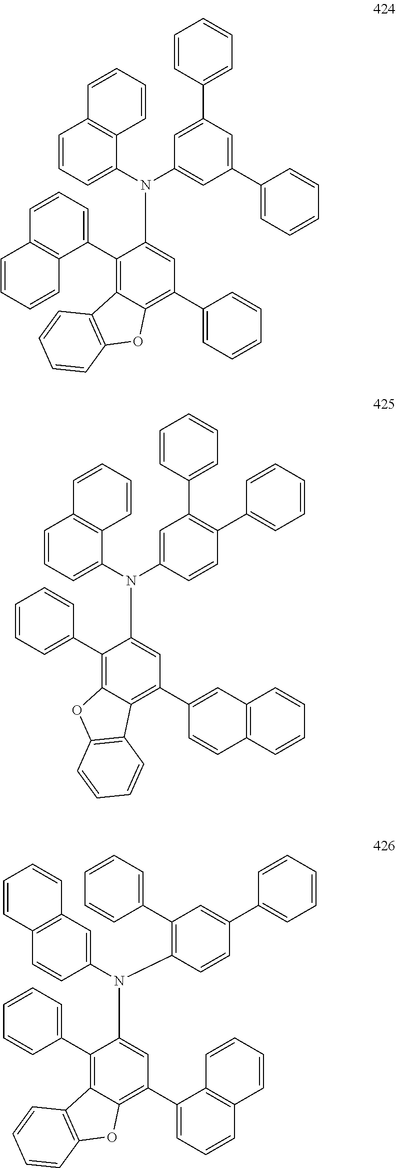 [SMILES: c1ccc(-c2cc(-c3ccccc3)cc(N(c3cc(-c4ccccc4)c4oc5ccccc5c4c3-c3cccc4ccccc34)c3cccc4ccccc34)c2)cc1 ; c1ccc(-c2ccc(N(c3cc(-c4ccc5ccccc5c4)c4c(oc5ccccc54)c3-c3ccccc3)c3cccc4ccccc34)cc2-c2ccccc2)cc1 ; c1ccc(-c2ccc(N(c3ccc4ccccc4c3)c3cc(-c4cccc5ccccc45)c4oc5ccccc5c4c3-c3ccccc3)c(-c3ccccc3)c2)cc1]

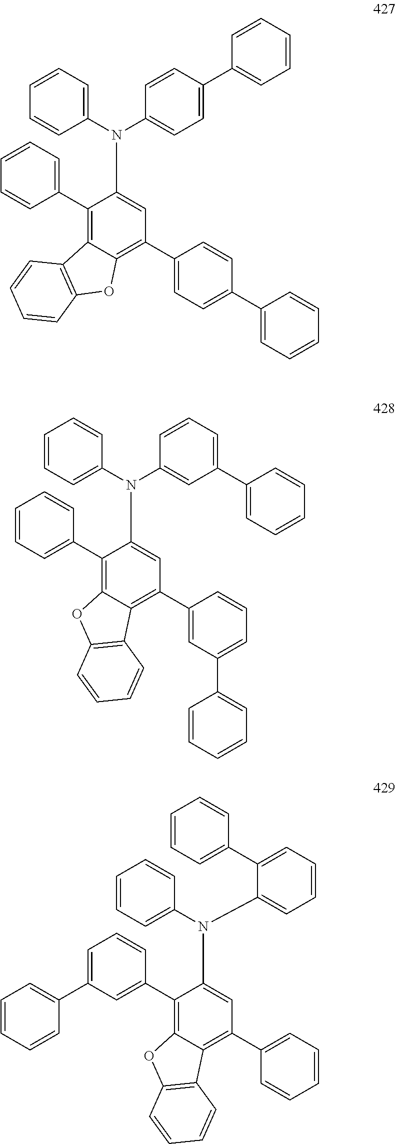

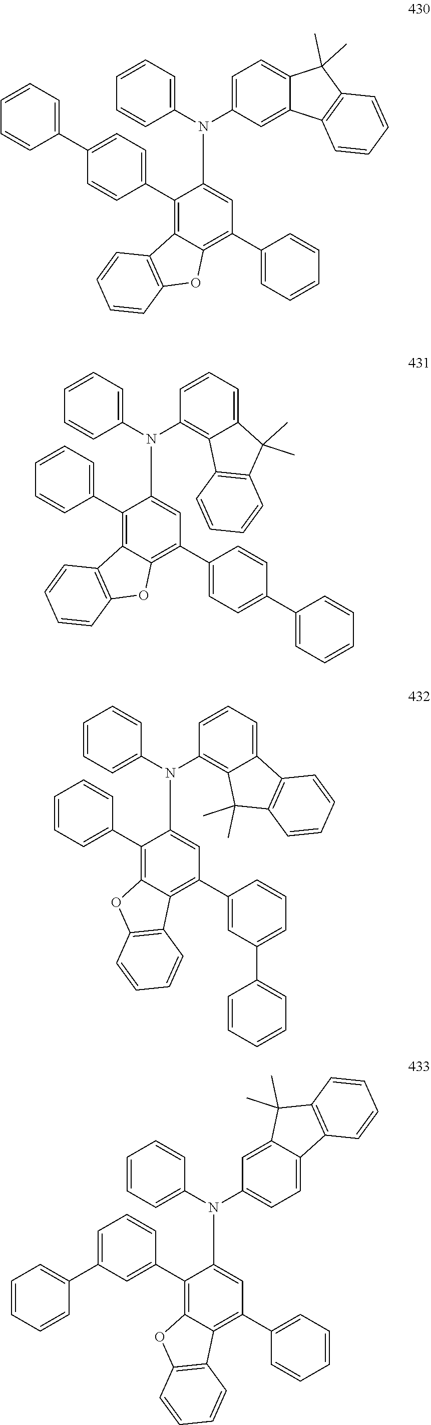

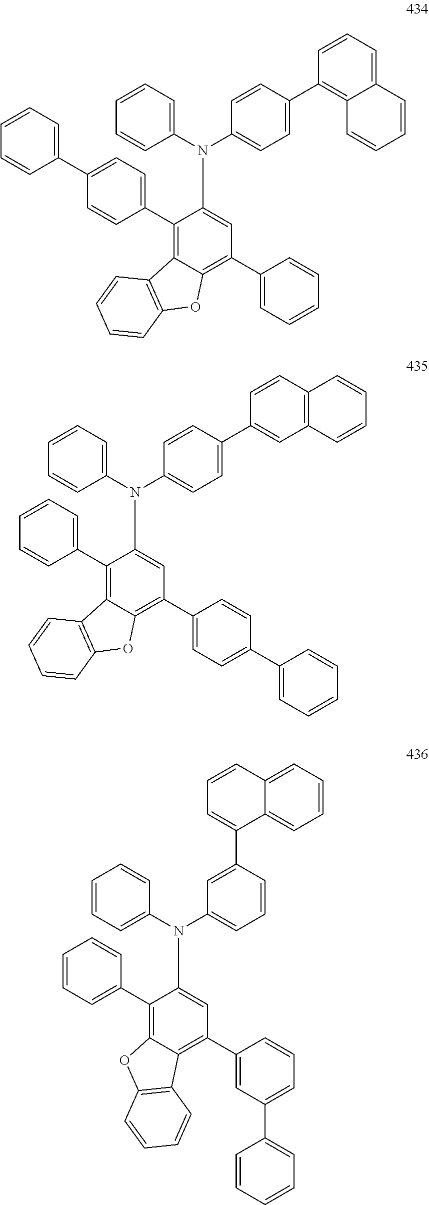 [SMILES: c1ccc(-c2ccc(-c3c(N(c4ccccc4)c4ccc(-c5cccc6ccccc56)cc4)cc(-c4ccccc4)c4oc5ccccc5c34)cc2)cc1 ; c1ccc(-c2ccc(-c3cc(N(c4ccccc4)c4ccc(-c5ccc6ccccc6c5)cc4)c(-c4ccccc4)c4c3oc3ccccc34)cc2)cc1 ; c1ccc(-c2cccc(-c3cc(N(c4ccccc4)c4cccc(-c5cccc6ccccc56)c4)c(-c4ccccc4)c4oc5ccccc5c34)c2)cc1]

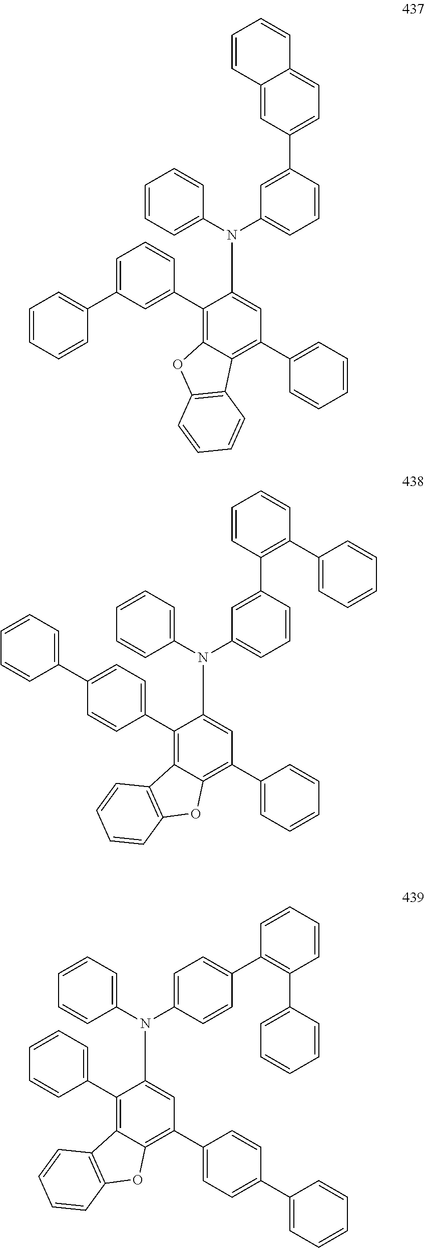 [SMILES: c1ccc(-c2cccc(-c3c(N(c4ccccc4)c4cccc(-c5ccc6ccccc6c5)c4)cc(-c4ccccc4)c4c3oc3ccccc34)c2)cc1 ; c1ccc(-c2ccc(-c3c(N(c4ccccc4)c4cccc(-c5ccccc5-c5ccccc5)c4)cc(-c4ccccc4)c4oc5ccccc5c34)cc2)cc1 ; c1ccc(-c2ccc(-c3cc(N(c4ccccc4)c4ccc(-c5ccccc5-c5ccccc5)cc4)c(-c4ccccc4)c4c3oc3ccccc34)cc2)cc1]

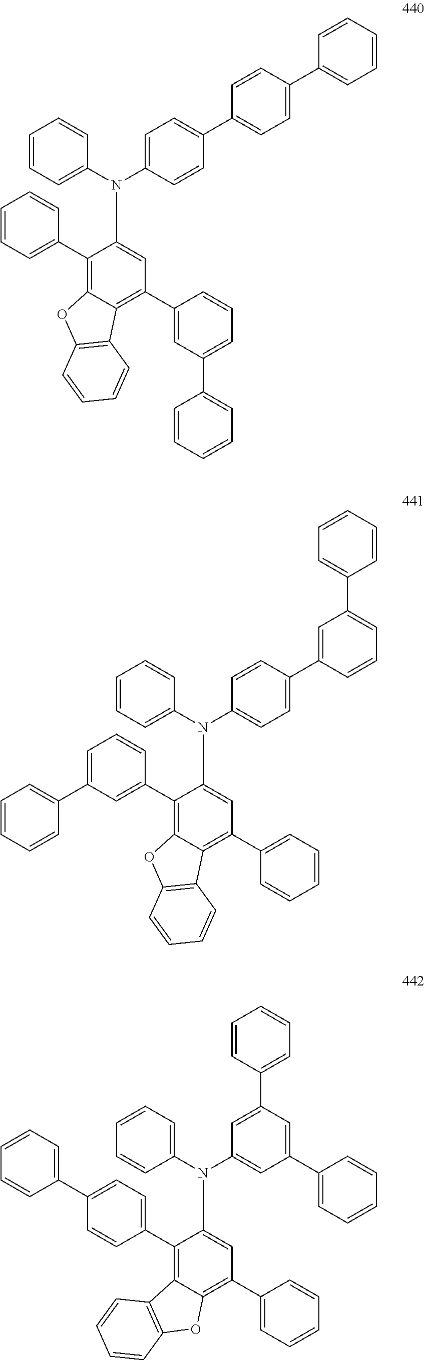 [SMILES: c1ccc(-c2ccc(-c3ccc(N(c4ccccc4)c4cc(-c5cccc(-c6ccccc6)c5)c5c(oc6ccccc65)c4-c4ccccc4)cc3)cc2)cc1 ; c1ccc(-c2cccc(-c3ccc(N(c4ccccc4)c4cc(-c5ccccc5)c5c(oc6ccccc65)c4-c4cccc(-c5ccccc5)c4)cc3)c2)cc1 ; c1ccc(-c2ccc(-c3c(N(c4ccccc4)c4cc(-c5ccccc5)cc(-c5ccccc5)c4)cc(-c4ccccc4)c4oc5ccccc5c34)cc2)cc1]

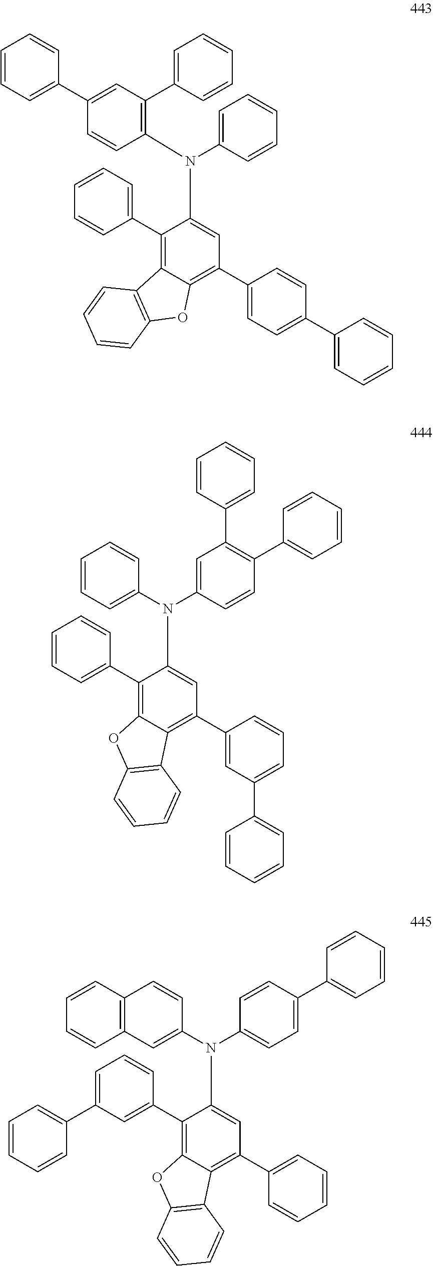 [SMILES: c1ccc(-c2ccc(-c3cc(N(c4ccccc4)c4ccc(-c5ccccc5)cc4-c4ccccc4)c(-c4ccccc4)c4c3oc3ccccc34)cc2)cc1 ; c1ccc(-c2cccc(-c3cc(N(c4ccccc4)c4ccc(-c5ccccc5)c(-c5ccccc5)c4)c(-c4ccccc4)c4oc5ccccc5c34)c2)cc1 ; c1ccc(-c2ccc(N(c3ccc4ccccc4c3)c3cc(-c4ccccc4)c4c(oc5ccccc54)c3-c3cccc(-c4ccccc4)c3)cc2)cc1]

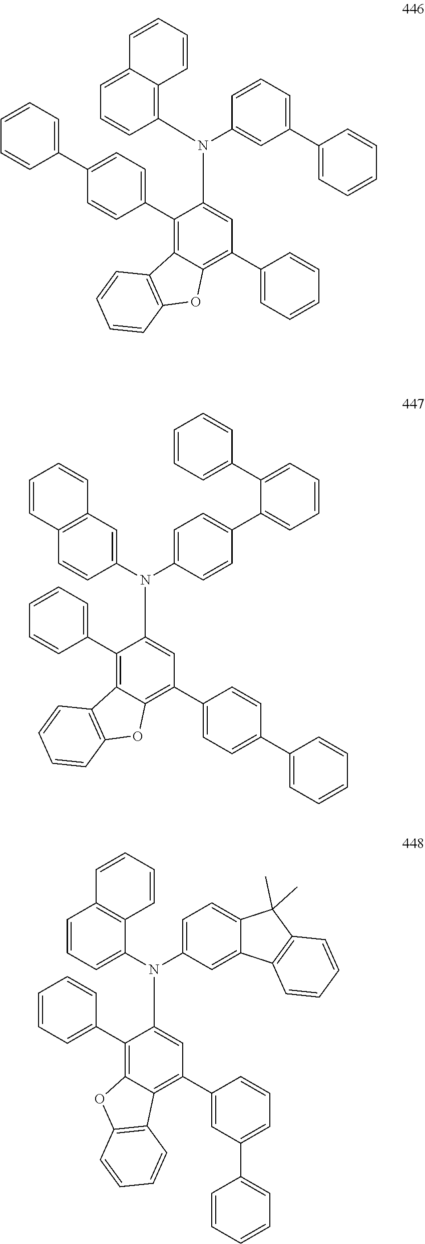 [SMILES: c1ccc(-c2ccc(-c3c(N(c4cccc(-c5ccccc5)c4)c4cccc5ccccc45)cc(-c4ccccc4)c4oc5ccccc5c34)cc2)cc1 ; c1ccc(-c2ccc(-c3cc(N(c4ccc(-c5ccccc5-c5ccccc5)cc4)c4ccc5ccccc5c4)c(-c4ccccc4)c4c3oc3ccccc34)cc2)cc1 ; CC1(C)c2ccccc2-c2cc(N(c3cc(-c4cccc(-c5ccccc5)c4)c4c(oc5ccccc54)c3-c3ccccc3)c3cccc4ccccc34)ccc21]

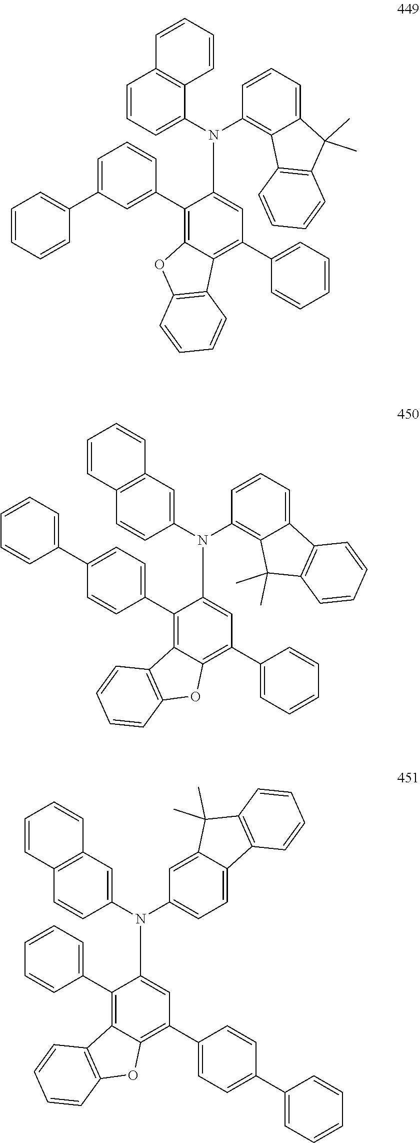 [SMILES: CC1(C)c2ccccc2-c2c(N(c3cc(-c4ccccc4)c4c(oc5ccccc54)c3-c3cccc(-c4ccccc4)c3)c3cccc4ccccc34)cccc21 ; CC1(C)c2ccccc2-c2cccc(N(c3ccc4ccccc4c3)c3cc(-c4ccccc4)c4oc5ccccc5c4c3-c3ccc(-c4ccccc4)cc3)c21 ; CC1(C)c2ccccc2-c2ccc(N(c3ccc4ccccc4c3)c3cc(-c4ccc(-c5ccccc5)cc4)c4oc5ccccc5c4c3-c3ccccc3)cc21]

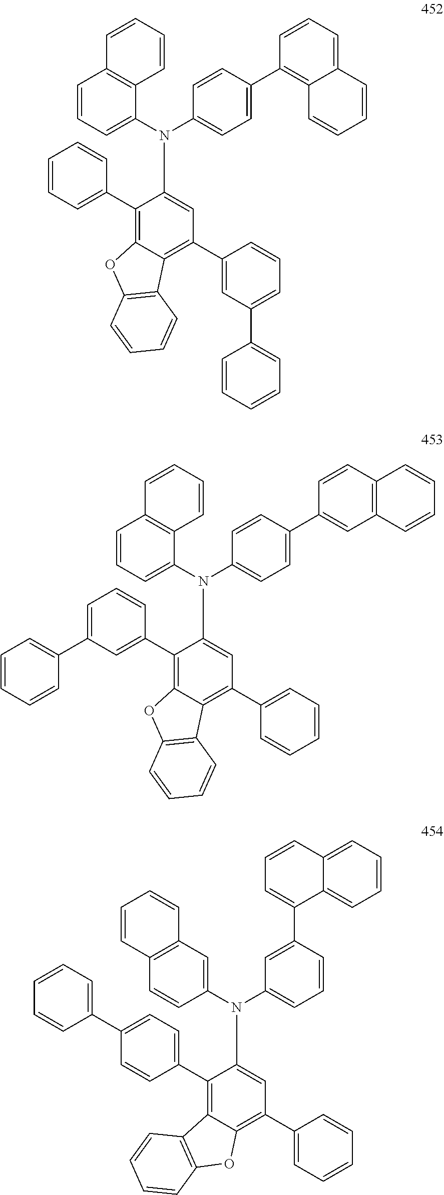 [SMILES: c1ccc(-c2cccc(-c3cc(N(c4ccc(-c5cccc6ccccc56)cc4)c4cccc5ccccc45)c(-c4ccccc4)c4oc5ccccc5c34)c2)cc1 ; c1ccc(-c2cccc(-c3c(N(c4ccc(-c5ccc6ccccc6c5)cc4)c4cccc5ccccc45)cc(-c4ccccc4)c4c3oc3ccccc34)c2)cc1 ; c1ccc(-c2ccc(-c3c(N(c4cccc(-c5cccc6ccccc56)c4)c4ccc5ccccc5c4)cc(-c4ccccc4)c4oc5ccccc5c34)cc2)cc1]

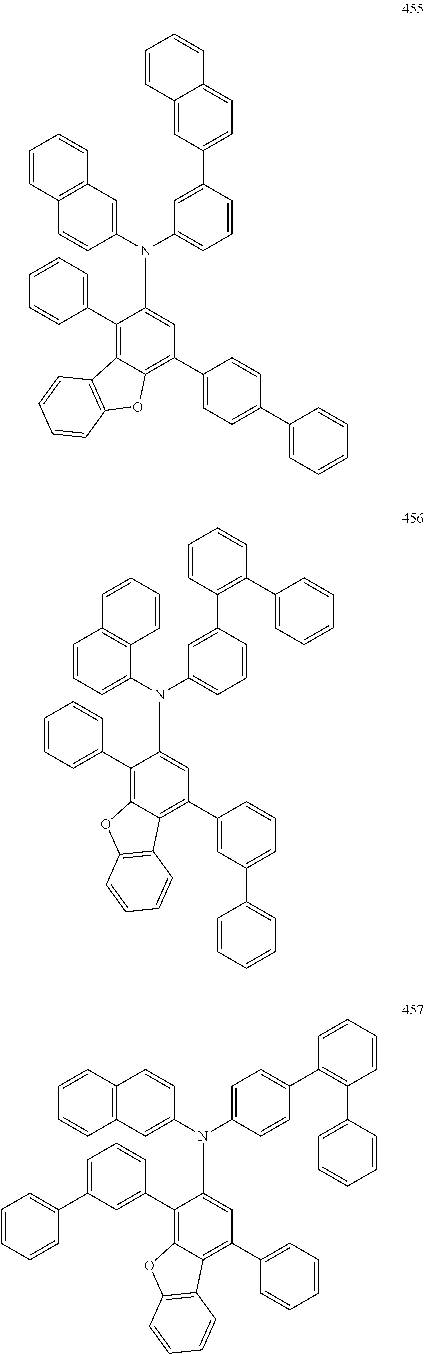 [SMILES: c1ccc(-c2ccc(-c3cc(N(c4cccc(-c5ccc6ccccc6c5)c4)c4ccc5ccccc5c4)c(-c4ccccc4)c4c3oc3ccccc34)cc2)cc1 ; c1ccc(-c2cccc(-c3cc(N(c4cccc(-c5ccccc5-c5ccccc5)c4)c4cccc5ccccc45)c(-c4ccccc4)c4oc5ccccc5c34)c2)cc1 ; c1ccc(-c2cccc(-c3c(N(c4ccc(-c5ccccc5-c5ccccc5)cc4)c4ccc5ccccc5c4)cc(-c4ccccc4)c4c3oc3ccccc34)c2)cc1]

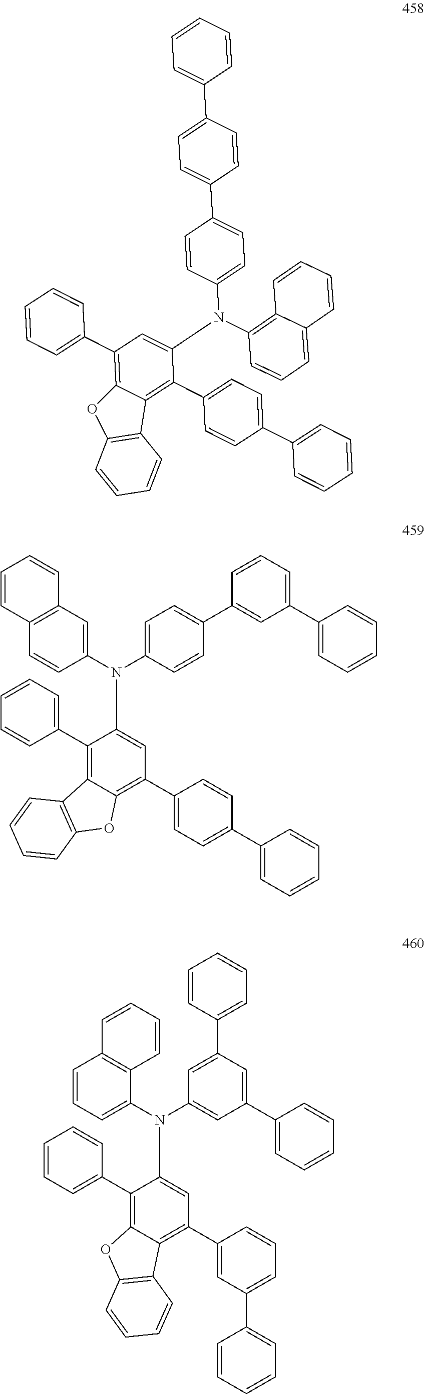 [SMILES: c1ccc(-c2ccc(-c3ccc(N(c4cc(-c5ccccc5)c5oc6ccccc6c5c4-c4ccc(-c5ccccc5)cc4)c4cccc5ccccc45)cc3)cc2)cc1 ; c1ccc(-c2ccc(-c3cc(N(c4ccc(-c5cccc(-c6ccccc6)c5)cc4)c4ccc5ccccc5c4)c(-c4ccccc4)c4c3oc3ccccc34)cc2)cc1 ; c1ccc(-c2cccc(-c3cc(N(c4cc(-c5ccccc5)cc(-c5ccccc5)c4)c4cccc5ccccc45)c(-c4ccccc4)c4oc5ccccc5c34)c2)cc1]

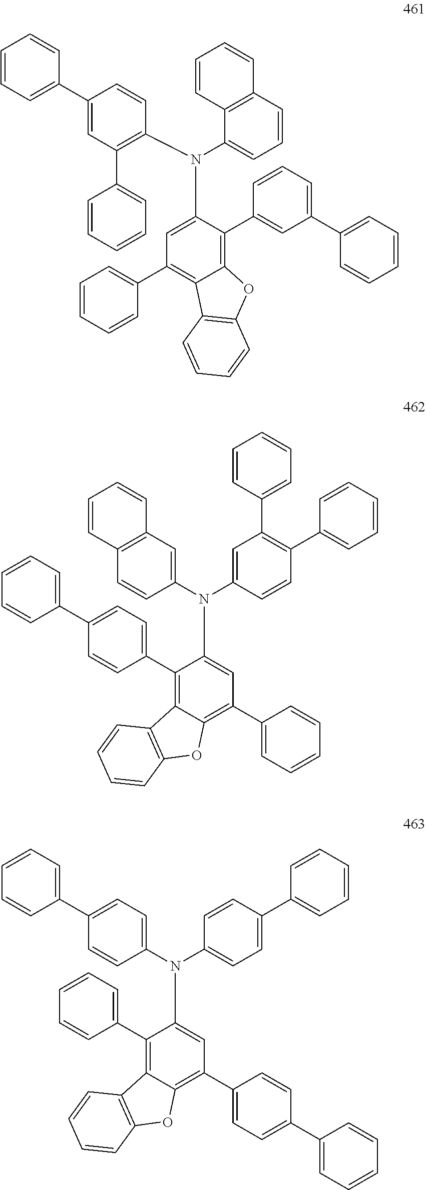 [SMILES: c1ccc(-c2cccc(-c3c(N(c4ccc(-c5ccccc5)cc4-c4ccccc4)c4cccc5ccccc45)cc(-c4ccccc4)c4c3oc3ccccc34)c2)cc1 ; c1ccc(-c2ccc(-c3c(N(c4ccc(-c5ccccc5)c(-c5ccccc5)c4)c4ccc5ccccc5c4)cc(-c4ccccc4)c4oc5ccccc5c34)cc2)cc1 ; c1ccc(-c2ccc(-c3cc(N(c4ccc(-c5ccccc5)cc4)c4ccc(-c5ccccc5)cc4)c(-c4ccccc4)c4c3oc3ccccc34)cc2)cc1]

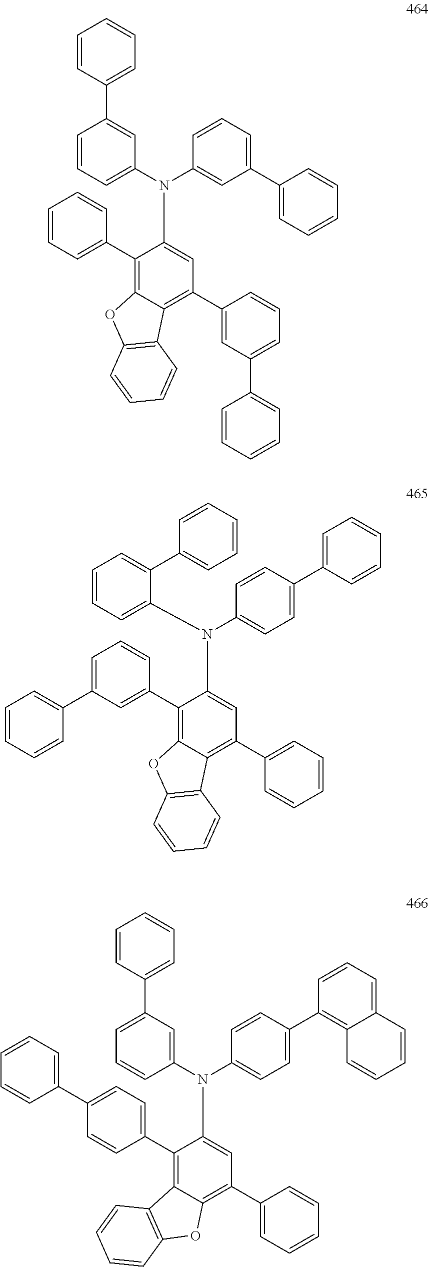 [SMILES: c1ccc(-c2cccc(-c3cc(N(c4cccc(-c5ccccc5)c4)c4cccc(-c5ccccc5)c4)c(-c4ccccc4)c4oc5ccccc5c34)c2)cc1 ; c1ccc(-c2ccc(N(c3ccccc3-c3ccccc3)c3cc(-c4ccccc4)c4c(oc5ccccc54)c3-c3cccc(-c4ccccc4)c3)cc2)cc1 ; c1ccc(-c2ccc(-c3c(N(c4ccc(-c5cccc6ccccc56)cc4)c4cccc(-c5ccccc5)c4)cc(-c4ccccc4)c4oc5ccccc5c34)cc2)cc1]

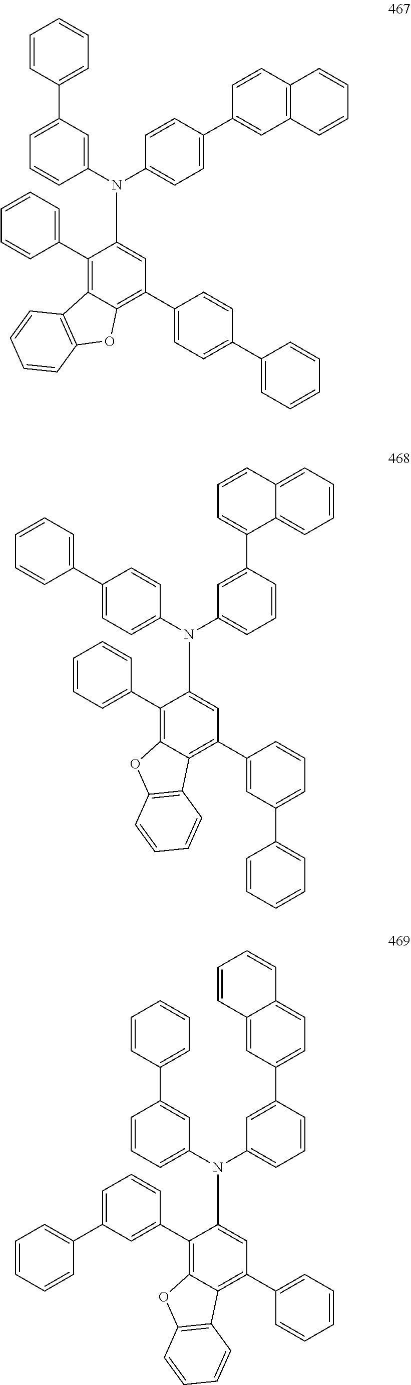 [SMILES: c1ccc(-c2ccc(-c3cc(N(c4ccc(-c5ccc6ccccc6c5)cc4)c4cccc(-c5ccccc5)c4)c(-c4ccccc4)c4c3oc3ccccc34)cc2)cc1 ; c1ccc(-c2ccc(N(c3cccc(-c4cccc5ccccc45)c3)c3cc(-c4cccc(-c5ccccc5)c4)c4c(oc5ccccc54)c3-c3ccccc3)cc2)cc1 ; c1ccc(-c2cccc(-c3c(N(c4cccc(-c5ccccc5)c4)c4cccc(-c5ccc6ccccc6c5)c4)cc(-c4ccccc4)c4c3oc3ccccc34)c2)cc1]

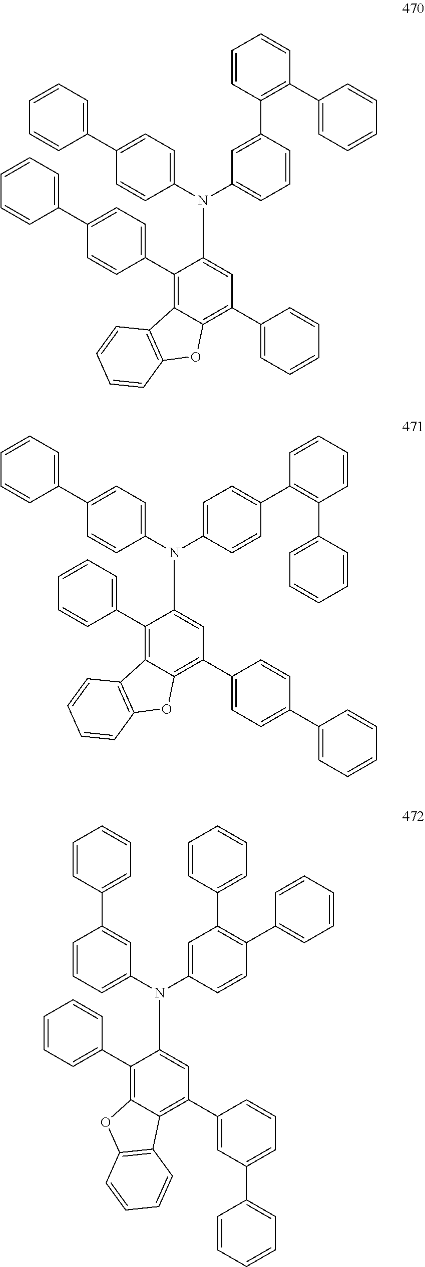 [SMILES: c1ccc(-c2ccc(-c3c(N(c4ccc(-c5ccccc5)cc4)c4cccc(-c5ccccc5-c5ccccc5)c4)cc(-c4ccccc4)c4oc5ccccc5c34)cc2)cc1 ; c1ccc(-c2ccc(-c3cc(N(c4ccc(-c5ccccc5)cc4)c4ccc(-c5ccccc5-c5ccccc5)cc4)c(-c4ccccc4)c4c3oc3ccccc34)cc2)cc1 ; c1ccc(-c2cccc(-c3cc(N(c4cccc(-c5ccccc5)c4)c4ccc(-c5ccccc5)c(-c5ccccc5)c4)c(-c4ccccc4)c4oc5ccccc5c34)c2)cc1]

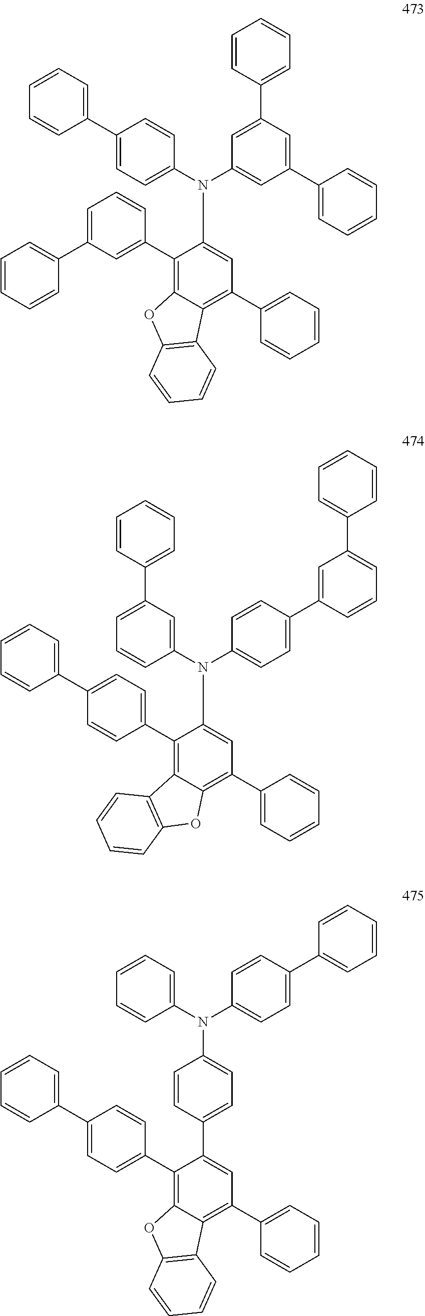 [SMILES: c1ccc(-c2ccc(N(c3cc(-c4ccccc4)cc(-c4ccccc4)c3)c3cc(-c4ccccc4)c4c(oc5ccccc54)c3-c3cccc(-c4ccccc4)c3)cc2)cc1 ; c1ccc(-c2ccc(-c3c(N(c4ccc(-c5cccc(-c6ccccc6)c5)cc4)c4cccc(-c5ccccc5)c4)cc(-c4ccccc4)c4oc5ccccc5c34)cc2)cc1 ; c1ccc(-c2ccc(-c3c(-c4ccc(N(c5ccccc5)c5ccc(-c6ccccc6)cc5)cc4)cc(-c4ccccc4)c4c3oc3ccccc34)cc2)cc1]

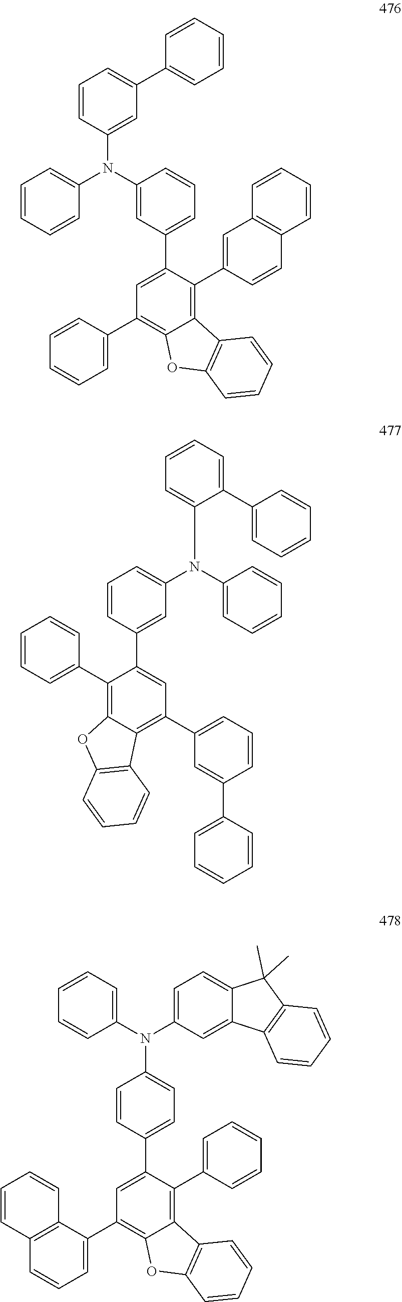 [SMILES: c1ccc(-c2cccc(N(c3ccccc3)c3cccc(-c4cc(-c5ccccc5)c5oc6ccccc6c5c4-c4ccc5ccccc5c4)c3)c2)cc1 ; c1ccc(-c2cccc(-c3cc(-c4cccc(N(c5ccccc5)c5ccccc5-c5ccccc5)c4)c(-c4ccccc4)c4oc5ccccc5c34)c2)cc1 ; CC1(C)c2ccccc2-c2cc(N(c3ccccc3)c3ccc(-c4cc(-c5cccc6ccccc56)c5oc6ccccc6c5c4-c4ccccc4)cc3)ccc21]

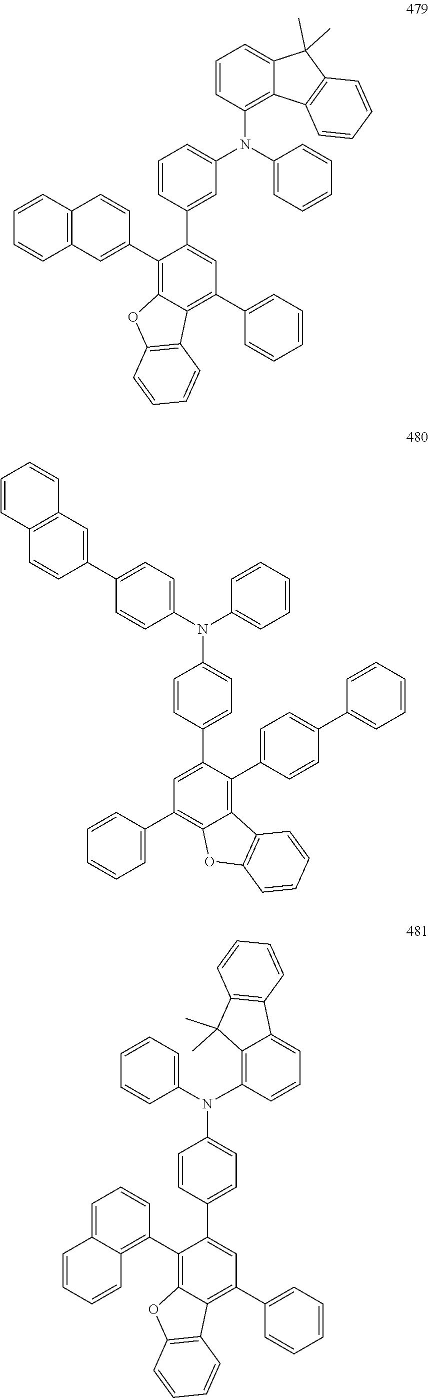 [SMILES: CC1(C)c2ccccc2-c2c(N(c3ccccc3)c3cccc(-c4cc(-c5ccccc5)c5c(oc6ccccc65)c4-c4ccc5ccccc5c4)c3)cccc21 ; c1ccc(-c2ccc(-c3c(-c4ccc(N(c5ccccc5)c5ccc(-c6ccc7ccccc7c6)cc5)cc4)cc(-c4ccccc4)c4oc5ccccc5c34)cc2)cc1 ; CC1(C)c2ccccc2-c2cccc(N(c3ccccc3)c3ccc(-c4cc(-c5ccccc5)c5c(oc6ccccc65)c4-c4cccc5ccccc45)cc3)c21]

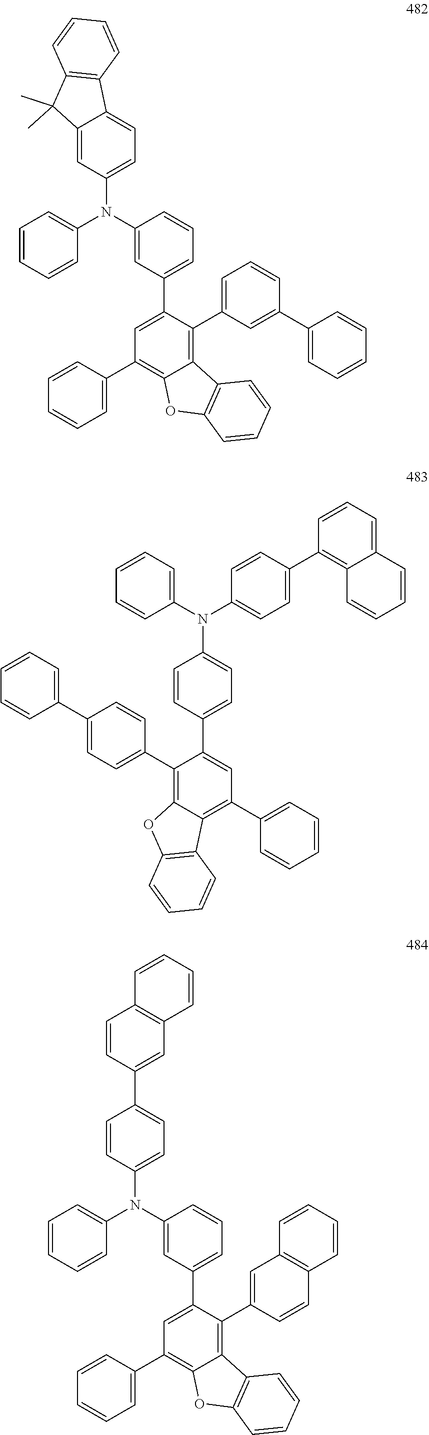

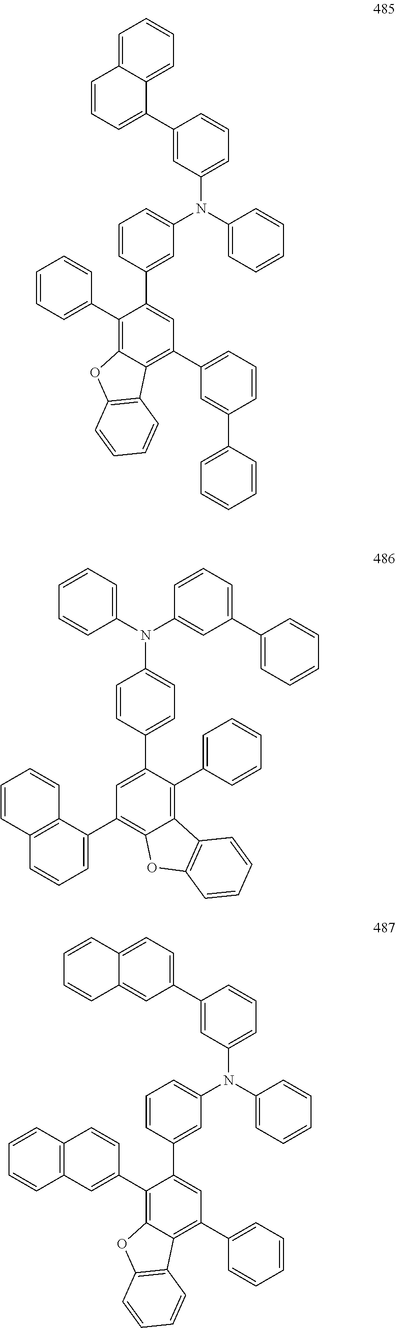 [SMILES: c1ccc(-c2cccc(-c3cc(-c4cccc(N(c5ccccc5)c5cccc(-c6cccc7ccccc67)c5)c4)c(-c4ccccc4)c4oc5ccccc5c34)c2)cc1 ; c1ccc(-c2cccc(N(c3ccccc3)c3ccc(-c4cc(-c5cccc6ccccc56)c5oc6ccccc6c5c4-c4ccccc4)cc3)c2)cc1 ; c1ccc(-c2cc(-c3cccc(N(c4ccccc4)c4cccc(-c5ccc6ccccc6c5)c4)c3)c(-c3ccc4ccccc4c3)c3oc4ccccc4c23)cc1]

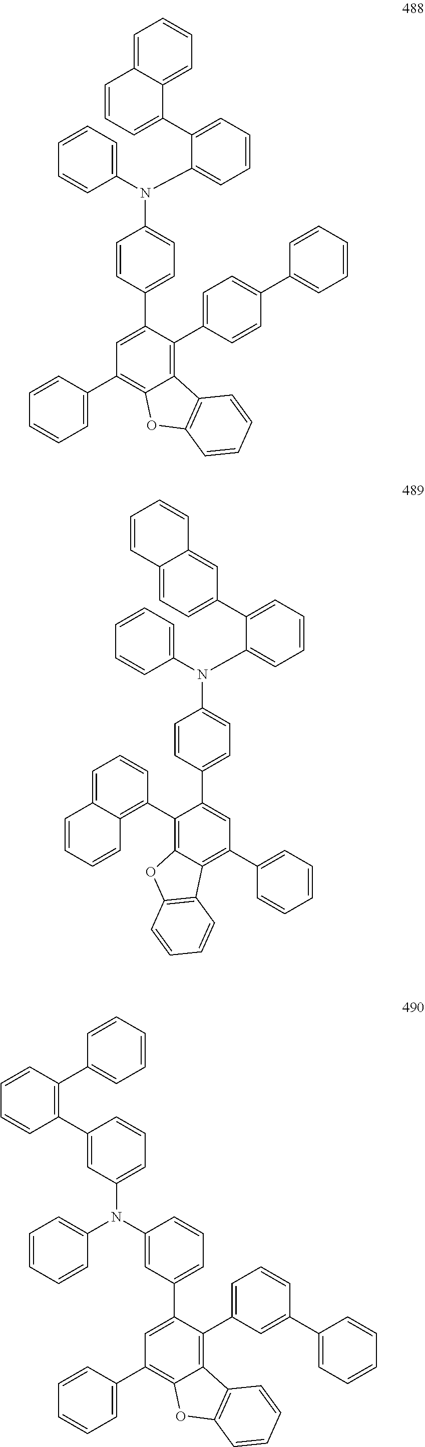 [SMILES: c1ccc(-c2ccc(-c3c(-c4ccc(N(c5ccccc5)c5ccccc5-c5cccc6ccccc56)cc4)cc(-c4ccccc4)c4oc5ccccc5c34)cc2)cc1 ; c1ccc(-c2cc(-c3ccc(N(c4ccccc4)c4ccccc4-c4ccc5ccccc5c4)cc3)c(-c3cccc4ccccc34)c3oc4ccccc4c23)cc1 ; c1ccc(-c2cccc(-c3c(-c4cccc(N(c5ccccc5)c5cccc(-c6ccccc6-c6ccccc6)c5)c4)cc(-c4ccccc4)c4oc5ccccc5c34)c2)cc1]

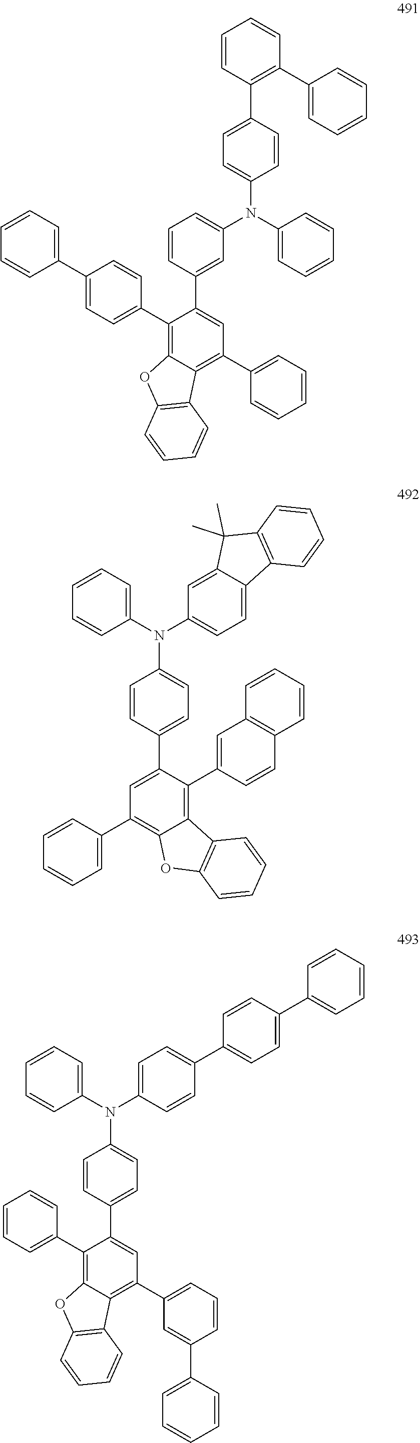 [SMILES: c1ccc(-c2ccc(-c3c(-c4cccc(N(c5ccccc5)c5ccc(-c6ccccc6-c6ccccc6)cc5)c4)cc(-c4ccccc4)c4c3oc3ccccc34)cc2)cc1 ; CC1(C)c2ccccc2-c2ccc(N(c3ccccc3)c3ccc(-c4cc(-c5ccccc5)c5oc6ccccc6c5c4-c4ccc5ccccc5c4)cc3)cc21 ; c1ccc(-c2ccc(-c3ccc(N(c4ccccc4)c4ccc(-c5cc(-c6cccc(-c7ccccc7)c6)c6c(oc7ccccc76)c5-c5ccccc5)cc4)cc3)cc2)cc1]

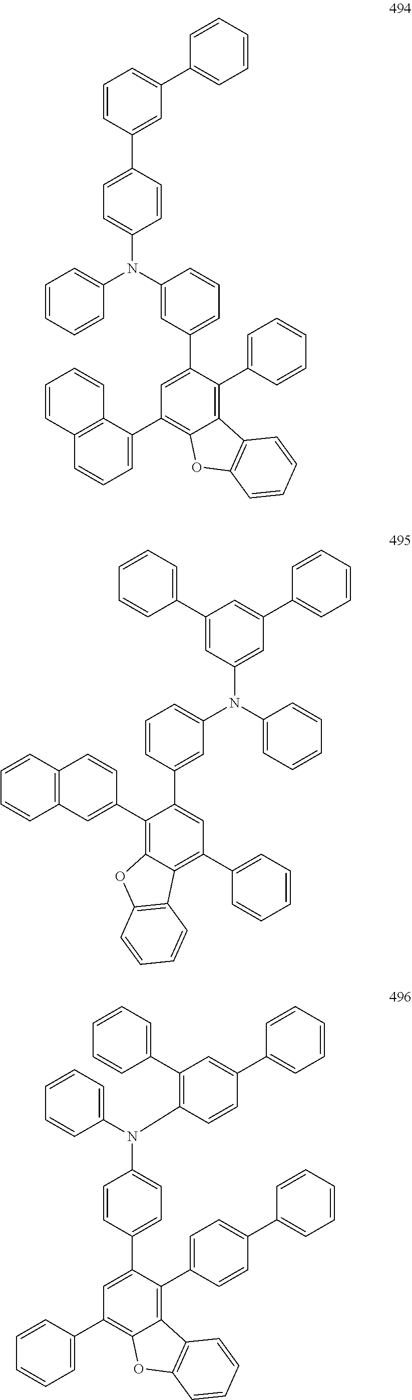

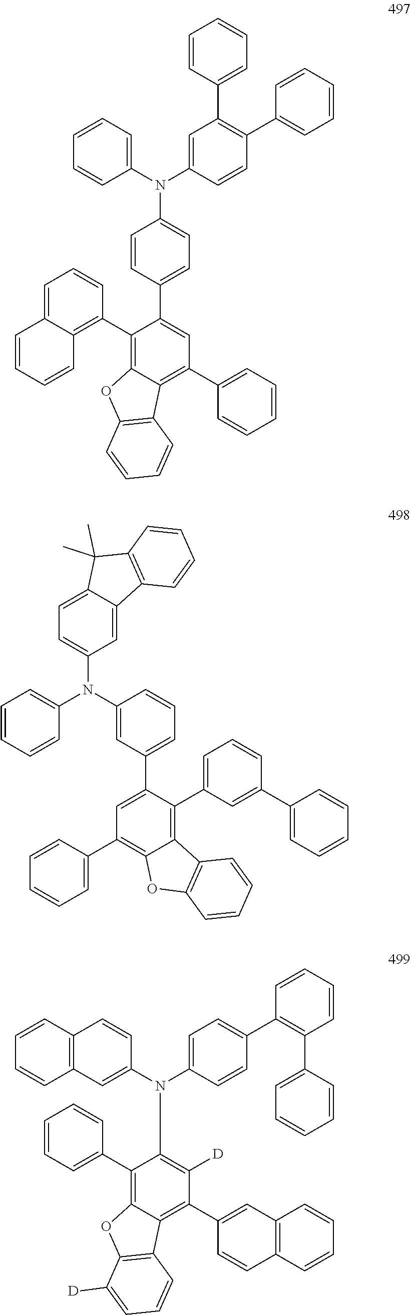 [SMILES: c1ccc(-c2ccc(N(c3ccccc3)c3ccc(-c4cc(-c5ccccc5)c5c(oc6ccccc65)c4-c4cccc5ccccc45)cc3)cc2-c2ccccc2)cc1 ; CC1(C)c2ccccc2-c2cc(N(c3ccccc3)c3cccc(-c4cc(-c5ccccc5)c5oc6ccccc6c5c4-c4cccc(-c5ccccc5)c4)c3)ccc21 ; [2H]c1c(N(c2ccc(-c3ccccc3-c3ccccc3)cc2)c2ccc3ccccc3c2)c(-c2ccccc2)c2oc3c([2H])cccc3c2c1-c1ccc2ccccc2c1]

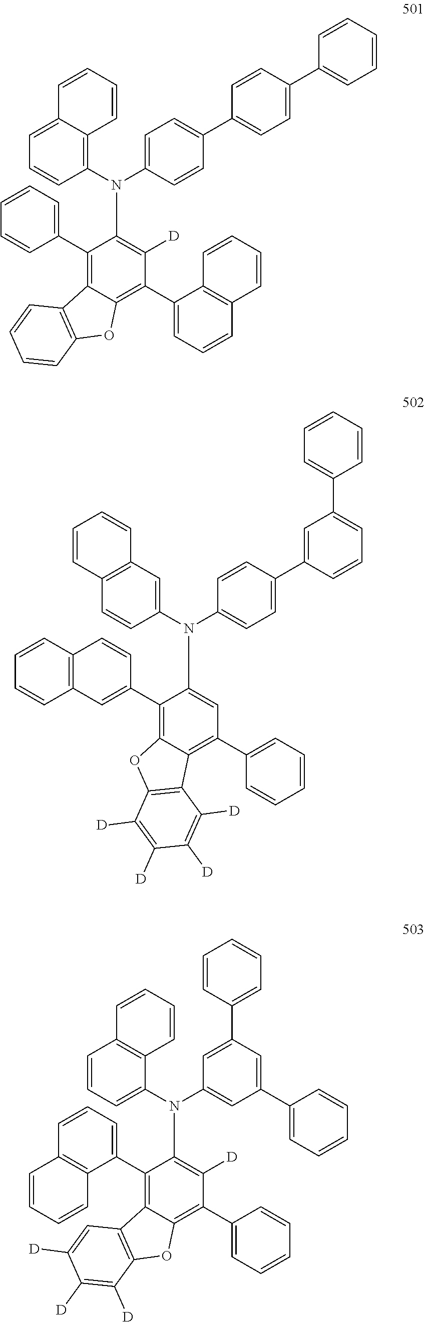 [SMILES: [2H]c1c(N(c2ccc(-c3ccc(-c4ccccc4)cc3)cc2)c2cccc3ccccc23)c(-c2ccccc2)c2c(oc3ccccc32)c1-c1cccc2ccccc12 ; [2H]c1c([2H])c([2H])c2c(oc3c(-c4ccc5ccccc5c4)c(N(c4ccc(-c5cccc(-c6ccccc6)c5)cc4)c4ccc5ccccc5c4)cc(-c4ccccc4)c32)c1[2H] ; [2H]c1cc2c(oc3c(-c4ccccc4)c([2H])c(N(c4cc(-c5ccccc5)cc(-c5ccccc5)c4)c4cccc5ccccc45)c(-c4cccc5ccccc45)c32)c([2H])c1[2H]]

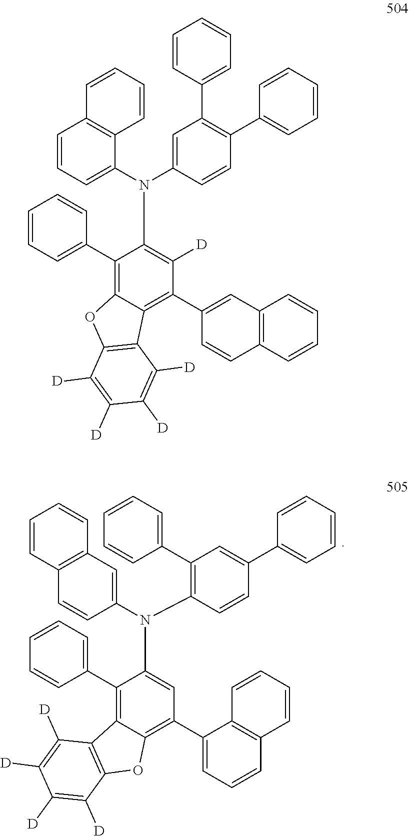 [SMILES: [2H]c1c([2H])c([2H])c2c(oc3c(-c4ccccc4)c(N(c4ccc(-c5ccccc5)c(-c5ccccc5)c4)c4cccc5ccccc45)c([2H])c(-c4ccc5ccccc5c4)c32)c1[2H] ; [2H]c1c([2H])c([2H])c2c(oc3c(-c4cccc5ccccc45)cc(N(c4ccc5ccccc5c4)c4ccc(-c5ccccc5)cc4-c4ccccc4)c(-c4ccccc4)c32)c1[2H]]

In a second aspect, the present disclosure provides an electronic element, comprising an anode and a cathode disposed opposite to each other, and a functional layer disposed between the anode and the cathode; the functional layer contains the organic compound of the present disclosure.

Optionally, the functional layer comprises an organic light-emitting layer and a hole transport layer, wherein the organic light-emitting layer comprises the organic compound and/or the hole transport layer comprises the organic compound.

Optionally, the electronic element is an organic electroluminescent device or a photoelectric conversion device.

Optionally, the electronic element is a red organic electroluminescent device.

Further optionally, the hole transport layer comprises a first hole transport layer and a second hole transport layer. The first hole transport layer is closer to the anode compared to the second hole transport layer, wherein the second hole transport layer comprises the organic compound of the present disclosure.

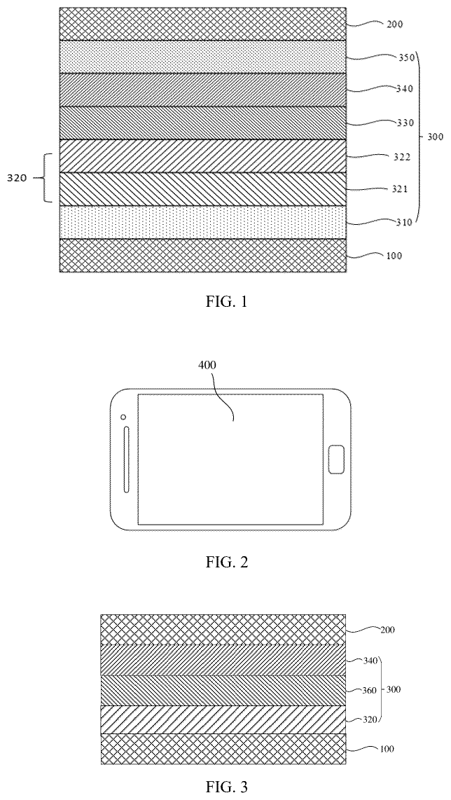

In an embodiment, the electronic element is an organic electroluminescent device. As shown in FIG. 1, the organic electroluminescent device may comprise an anode 100, a first hole transport layer 321, a second hole transport layer 322, an organic light-emitting layer 330, an electron transport layer 340, and a cathode 200 that are stacked. Among them, the first hole transport layer 321 and the second hole transport layer 322 constitute a hole transport layer 320.

Optionally, the anode 100 comprises an anode material as follows, which is preferably a high work function material contributing to injection of holes into the functional layer. Specific examples of the anode material include: metals such as nickel, platinum, vanadium, chromium, copper, zinc and gold, or alloys thereof; metal oxides such as zinc oxide, indium oxide, indium tin oxide (ITO), and indium zinc oxide (IZO); combinations of metals and oxides, such as ZnO:Al or SnO₂:Sb; and conductive polymers such as poly(3-methylthiophene), poly[3,4-(ethylene-1,2-dioxy)thiophene] (PEDT), polypyrrole, and polyaniline, but are not limited thereto. Preferably, a transparent electrode comprising indium tin oxide (ITO) as the anode is included.

Optionally, the hole transport layer includes one or more hole transport materials. The hole transport materials may be selected from carbazole polymers, carbazole-connected triarylamine compounds, and other types of compounds. It is not particularly defined in the present disclosure. For example, the materials of the first hole transport layer are selected from the group consisting of the following compounds:

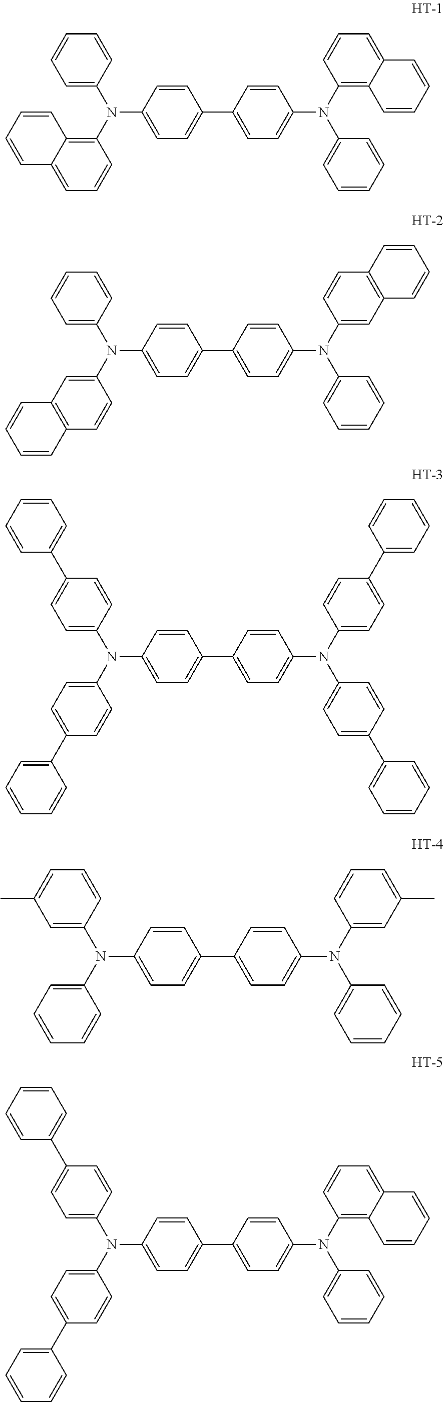

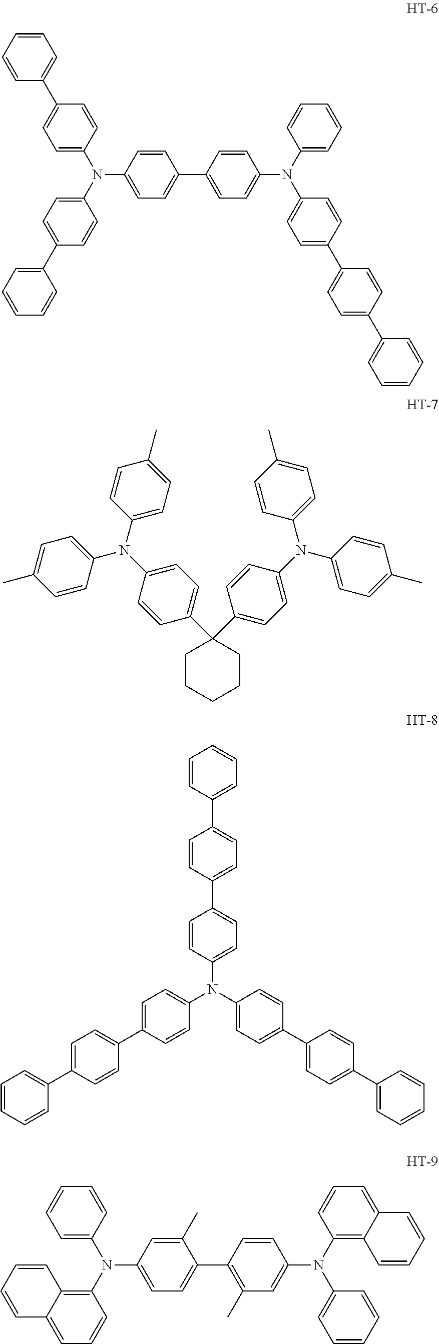

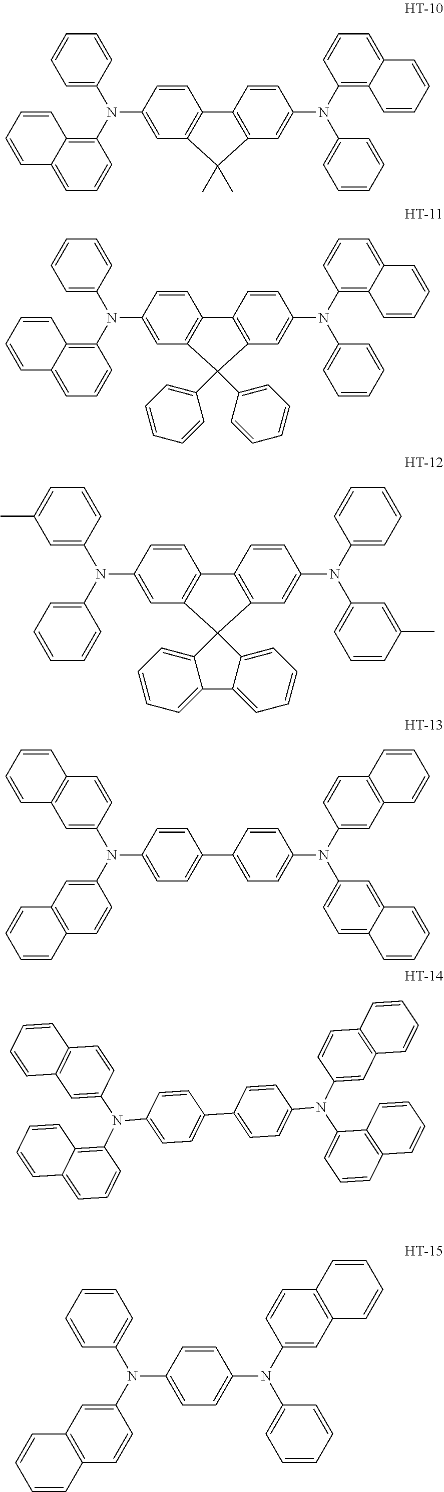

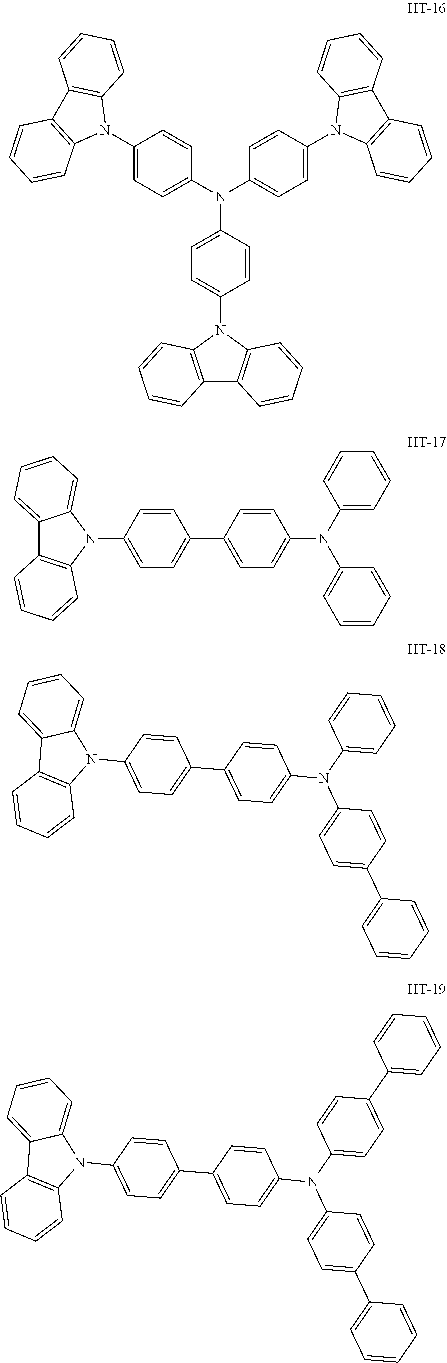 [SMILES: c1ccc2c(c1)c1ccccc1n2-c1ccc(N(c2ccc(-n3c4ccccc4c4ccccc43)cc2)c2ccc(-n3c4ccccc4c4ccccc43)cc2)cc1 ; c1ccc(N(c2ccccc2)c2ccc(-c3ccc(-n4c5ccccc5c5ccccc54)cc3)cc2)cc1 ; c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

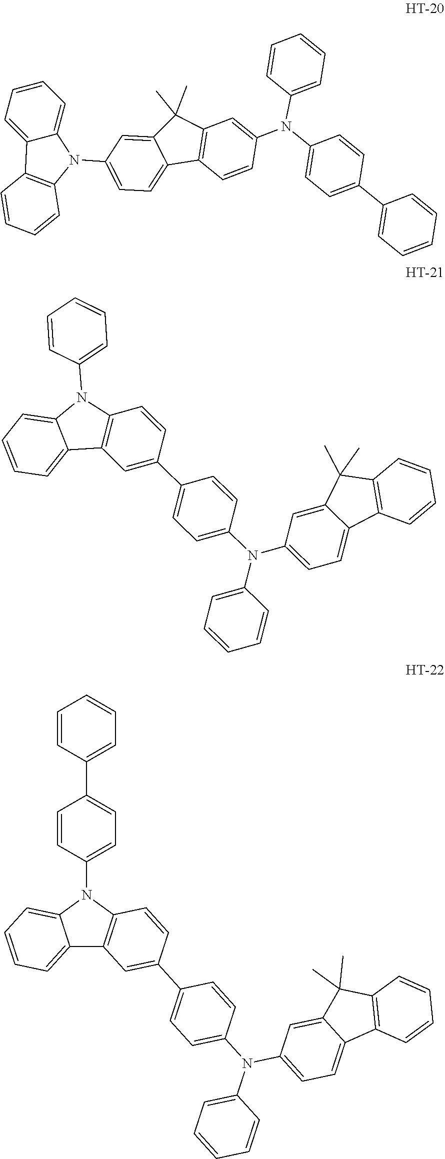 [SMILES: CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

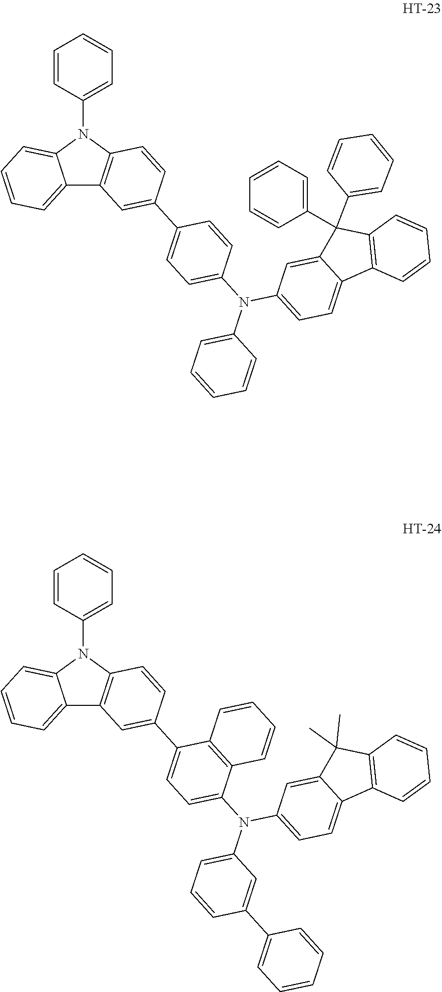 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21]

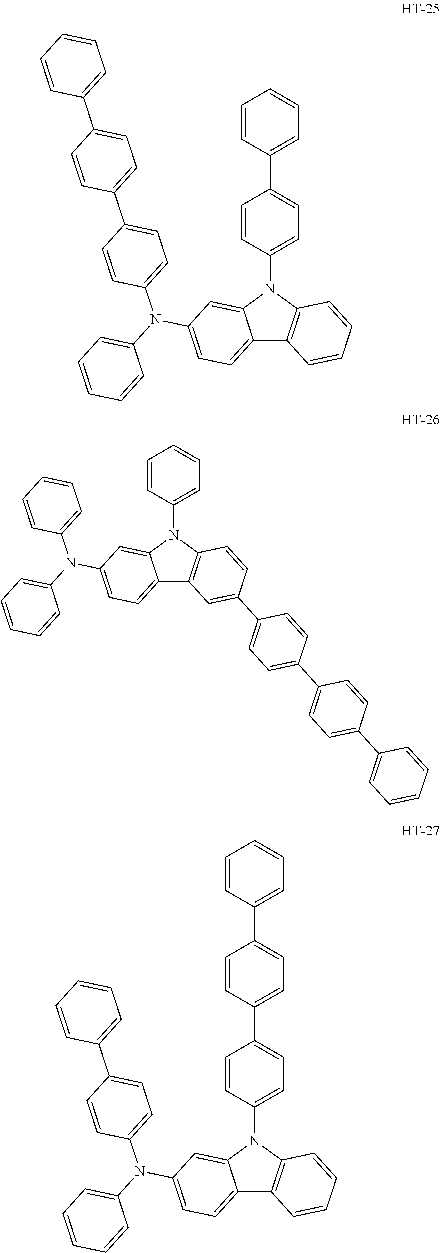 [SMILES: c1ccc(-c2ccc(-c3ccc(N(c4ccccc4)c4ccc5c6ccccc6n(-c6ccc(-c7ccccc7)cc6)c5c4)cc3)cc2)cc1 ; c1ccc(-c2ccc(-c3ccc(-c4ccc5c(c4)c4ccc(N(c6ccccc6)c6ccccc6)cc4n5-c4ccccc4)cc3)cc2)cc1 ; c1ccc(-c2ccc(-c3ccc(-n4c5ccccc5c5ccc(N(c6ccccc6)c6ccc(-c7ccccc7)cc6)cc54)cc3)cc2)cc1]

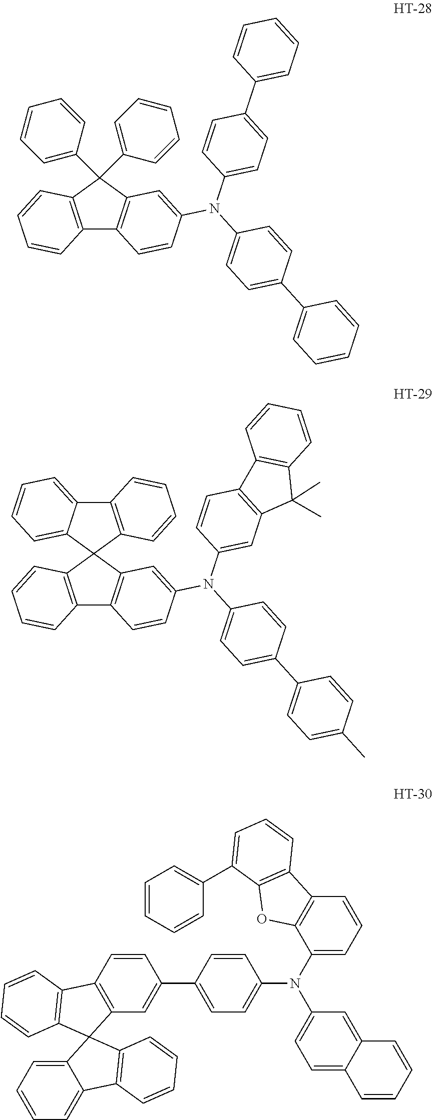 [SMILES: c1ccc(-c2ccc(N(c3ccc(-c4ccccc4)cc3)c3ccc4c(c3)C(c3ccccc3)(c3ccccc3)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

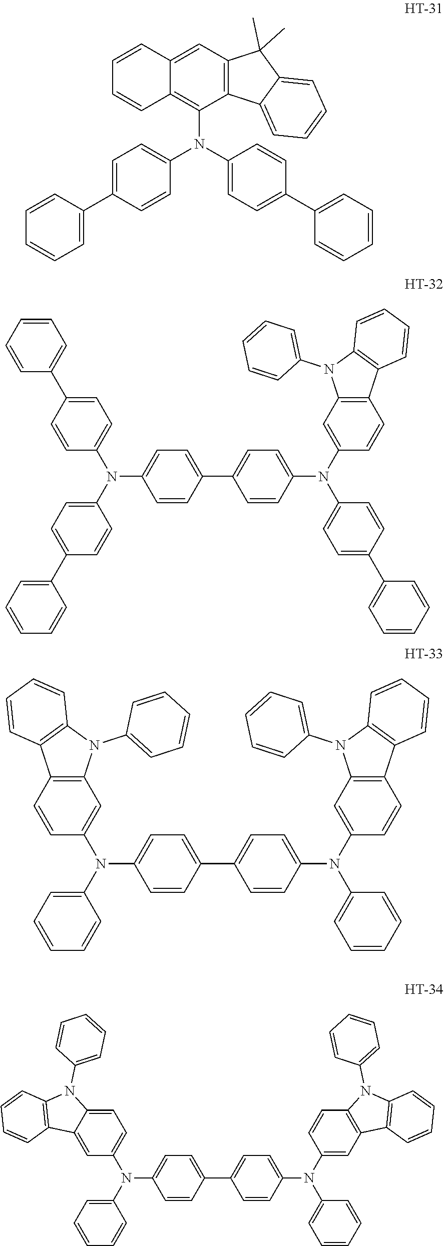 [SMILES: CC1(C)c2ccccc2-c2c1cc1ccccc1c2N(c1ccc(-c2ccccc2)cc1)c1ccc(-c2ccccc2)cc1 ; c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(N(c2ccc(-c3ccc(N(c4ccccc4)c4ccc5c6ccccc6n(-c6ccccc6)c5c4)cc3)cc2)c2ccc3c4ccccc4n(-c4ccccc4)c3c2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

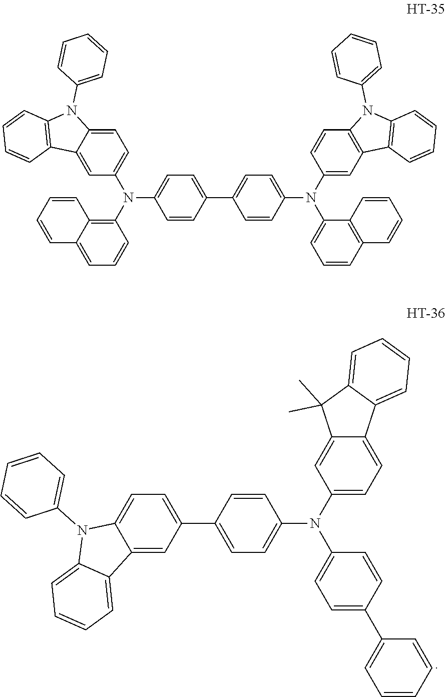 [SMILES: c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21]

In a specific embodiment, the first hole transport layer 321 is HT-36; and the second hole transport layer 322 is HT-31.

In another specific embodiment, the first hole transport layer 321 is HT-36; and the second hold transport layer 322 is the compound of the present disclosure.

Optionally, the organic light-emitting layer 330 may be composed of a single luminescent material or may comprise a host material and a dopant material. Optionally, the organic light-emitting layer 330 is composed of a host material and a dopant material. The holes injected into the organic light-emitting layer 330 and the electrons injected into the organic light-emitting layer 330 can recombine in the organic light-emitting layer 330 to form excitons. The excitons transmit energy to the host material, and the host material transmits the energy to the dopant material, thereby enabling the dopant material to emit light.

The host material of the organic light-emitting layer 330 may be a metal chelating compound, a stilbene-based derivative, an aromatic amine derivative, a dibenzofuran derivative, or other types of material. It is not particularly defined in the present disclosure. The host material may be a single host material, or a mixed host material. In an embodiment of the present disclosure, the host material of the organic light-emitting layer 330 is the compound of the present disclosure and RH-N. In an another embodiment of the present disclosure, the host material of the organic light-emitting layer 330 is RH-P and RH-N.

The dopant material of the organic light-emitting layer 330 can be selected based on existing technology, and for example, may be selected from an iridium (III) organometallic complex, a platinum (II) organometallic complex, a ruthenium (II) complex, etc. The specific examples of the dopant material include but are not limited to,

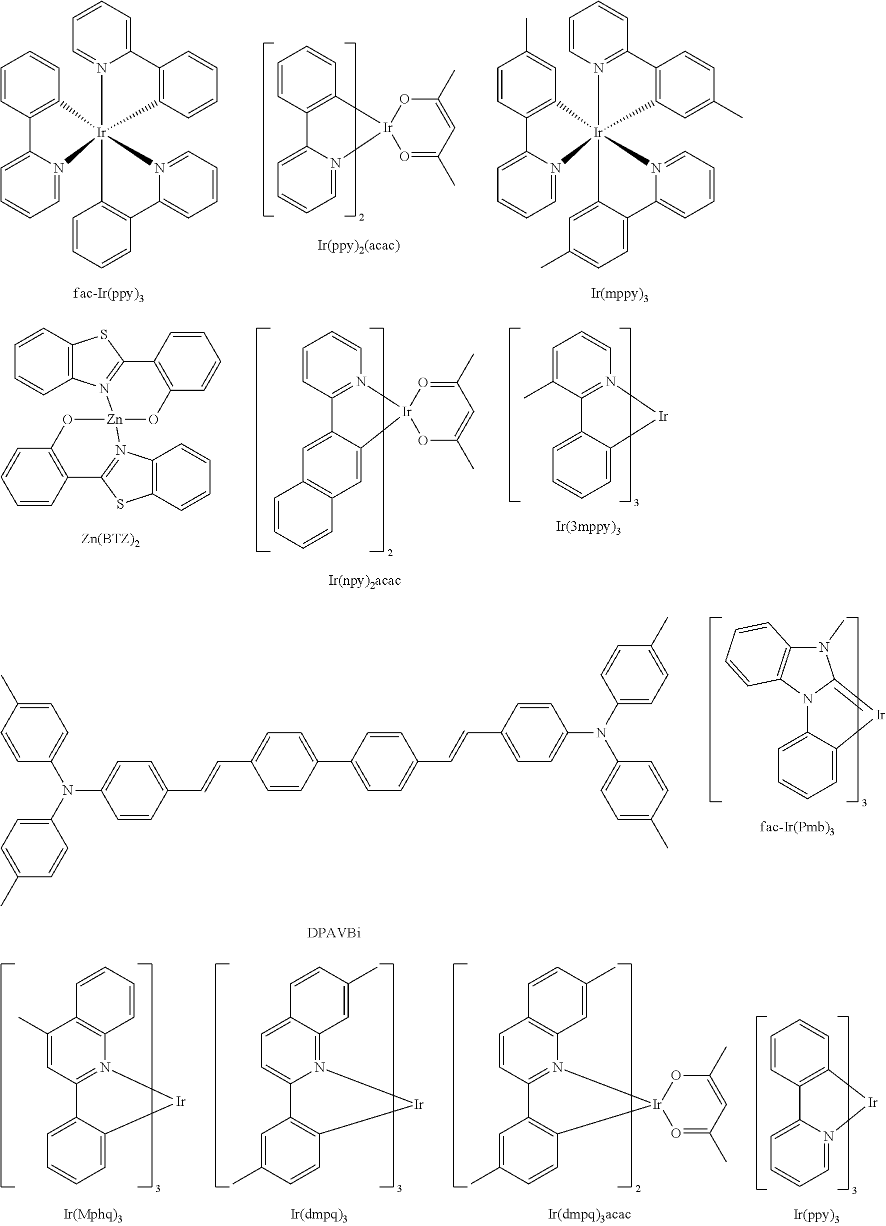

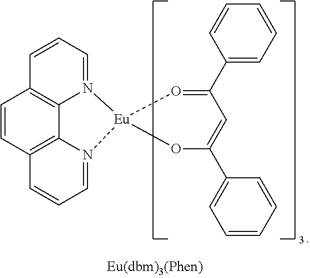

In an embodiment of the present disclosure, the dopant material of the organic light-emitting layer 330 is RD

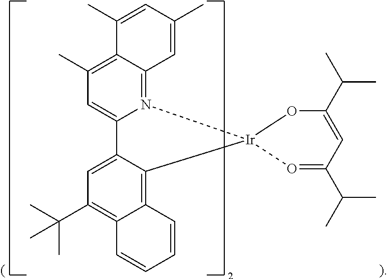

Optionally, the electron transport layer 340 may be a single-layer structure or a multilayer structure and may comprise one or more electron transport materials. The electron transport material typically includes a metal complex or a nitrogen-containing heterocyclic derivative, in which the metal complex material can be selected from LiQ, Alq₃, etc; the nitrogen-containing heterocyclic derivative can be an aromatic ring with a nitrogen-containing 5- or 6-membered ring skeleton, a fused aromatic ring compound with a nitrogen-containing 5- or 6-membered ring skeleton, etc. Specific examples include but are not limited to 1,10-phenanthroline compounds such as Bphen, NBphen, ET-21, BimiBphen, etc, or anthracene compounds containing heteroazoaryl, triazine compounds, or pyrimidine compounds as shown below. In an embodiment of the present disclosure, the electron transport layer 340 is composed of ET-21 and LiQ.

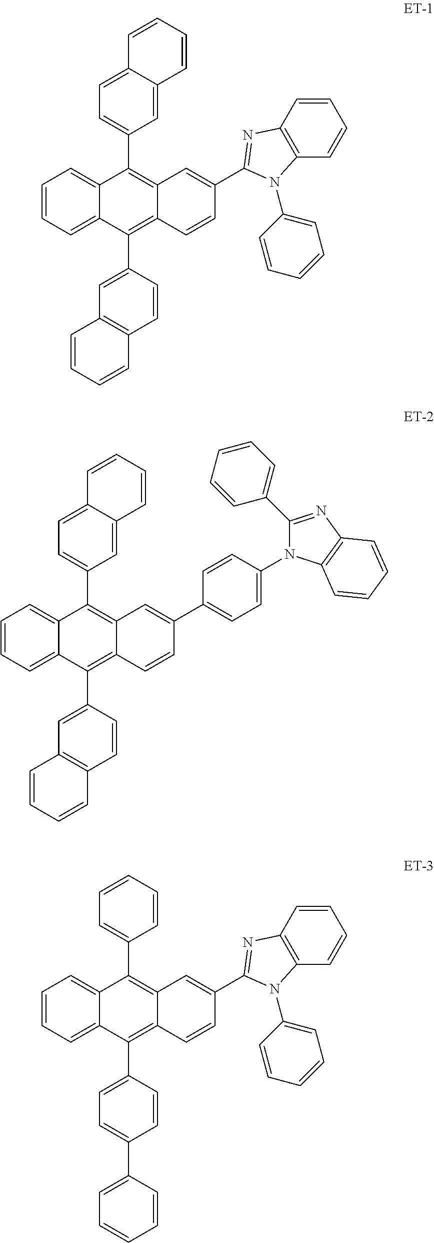 [SMILES: c1ccc(-n2c(-c3ccc4c(-c5ccc6ccccc6c5)c5ccccc5c(-c5ccc6ccccc6c5)c4c3)nc3ccccc32)cc1 ; c1ccc(-c2nc3ccccc3n2-c2ccc(-c3ccc4c(-c5ccc6ccccc6c5)c5ccccc5c(-c5ccc6ccccc6c5)c4c3)cc2)cc1 ; c1ccc(-c2ccc(-c3c4ccccc4c(-c4ccccc4)c4cc(-c5nc6ccccc6n5-c5ccccc5)ccc34)cc2)cc1]

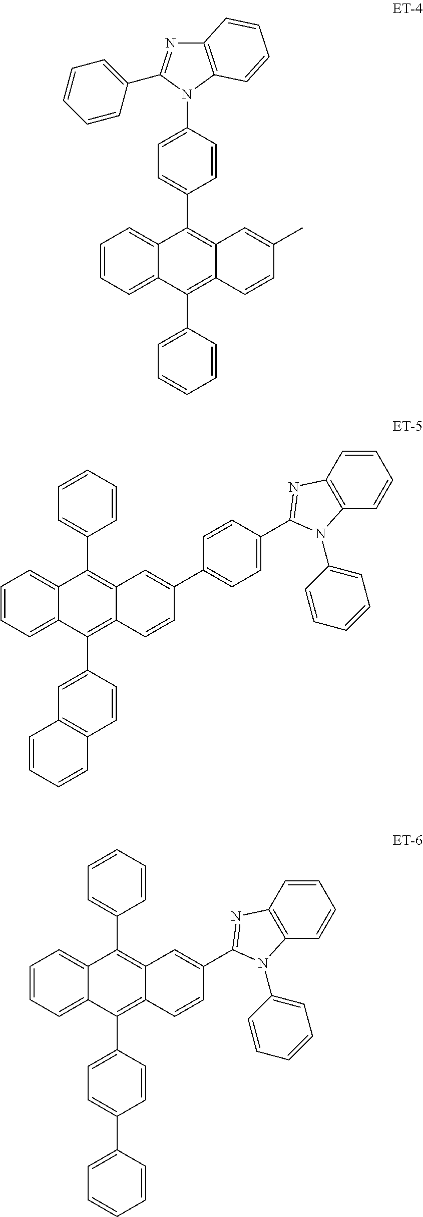 [SMILES: Cc1ccc2c(-c3ccccc3)c3ccccc3c(-c3ccc(-n4c(-c5ccccc5)nc5ccccc54)cc3)c2c1 ; c1ccc(-c2c3ccccc3c(-c3ccc4ccccc4c3)c3ccc(-c4ccc(-c5nc6ccccc6n5-c5ccccc5)cc4)cc23)cc1 ; c1ccc(-c2ccc(-c3c4ccccc4c(-c4ccccc4)c4cc(-c5nc6ccccc6n5-c5ccccc5)ccc34)cc2)cc1]

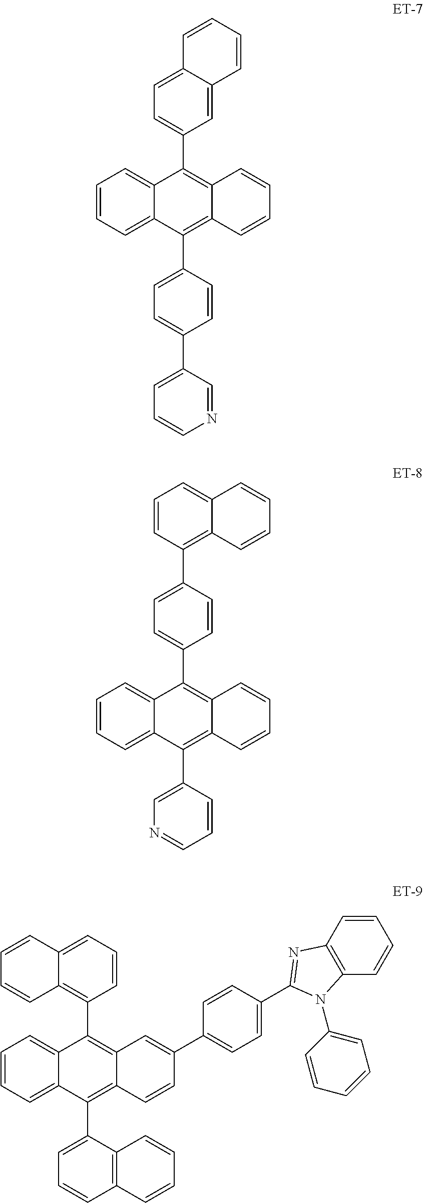 [SMILES: c1cncc(-c2ccc(-c3c4ccccc4c(-c4ccc5ccccc5c4)c4ccccc34)cc2)c1 ; c1cncc(-c2c3ccccc3c(-c3ccc(-c4cccc5ccccc45)cc3)c3ccccc23)c1 ; c1ccc(-n2c(-c3ccc(-c4ccc5c(-c6cccc7ccccc67)c6ccccc6c(-c6cccc7ccccc67)c5c4)cc3)nc3ccccc32)cc1]

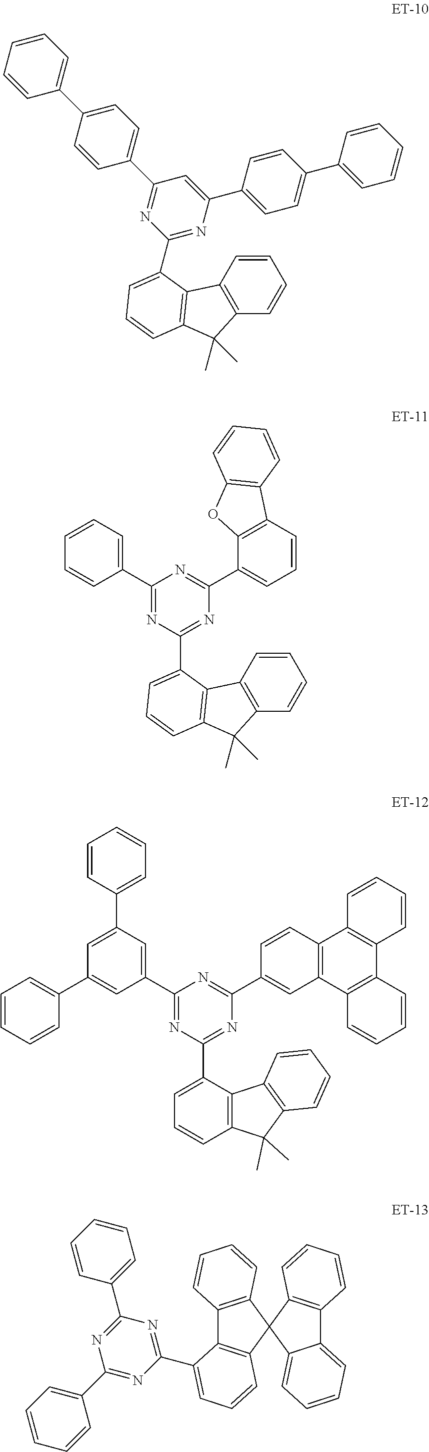 [SMILES: CC1(C)c2ccccc2-c2c(-c3nc(-c4ccc(-c5ccccc5)cc4)cc(-c4ccc(-c5ccccc5)cc4)n3)cccc21 ; CC1(C)c2ccccc2-c2c(-c3nc(-c4ccccc4)nc(-c4cccc5c4oc4ccccc45)n3)cccc21 ; CC1(C)c2ccccc2-c2c(-c3nc(-c4cc(-c5ccccc5)cc(-c5ccccc5)c4)nc(-c4ccc5c6ccccc6c6ccccc6c5c4)n3)cccc21 ; c1ccc(-c2nc(-c3ccccc3)nc(-c3cccc4c3-c3ccccc3C43c4ccccc4-c4ccccc43)n2)cc1]

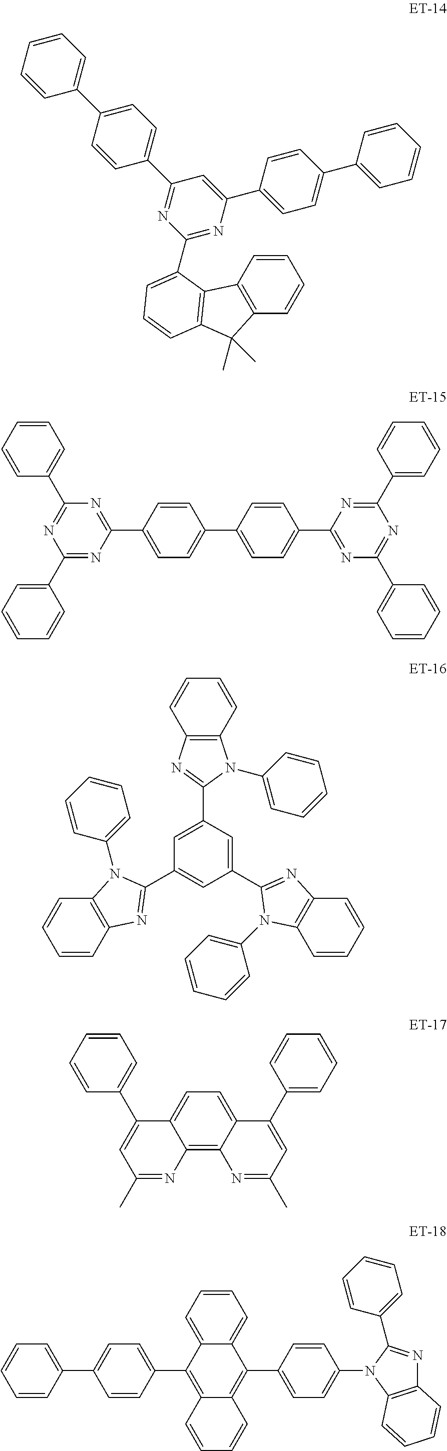 [SMILES: CC1(C)c2ccccc2-c2c(-c3nc(-c4ccc(-c5ccccc5)cc4)cc(-c4ccc(-c5ccccc5)cc4)n3)cccc21 ; c1ccc(-c2nc(-c3ccccc3)nc(-c3ccc(-c4ccc(-c5nc(-c6ccccc6)nc(-c6ccccc6)n5)cc4)cc3)n2)cc1 ; c1ccc(-n2c(-c3cc(-c4nc5ccccc5n4-c4ccccc4)cc(-c4nc5ccccc5n4-c4ccccc4)c3)nc3ccccc32)cc1 ; Cc1cc(-c2ccccc2)c2ccc3c(-c4ccccc4)cc(C)nc3c2n1 ; c1ccc(-c2ccc(-c3c4ccccc4c(-c4ccc(-n5c(-c6ccccc6)nc6ccccc65)cc4)c4ccccc34)cc2)cc1]

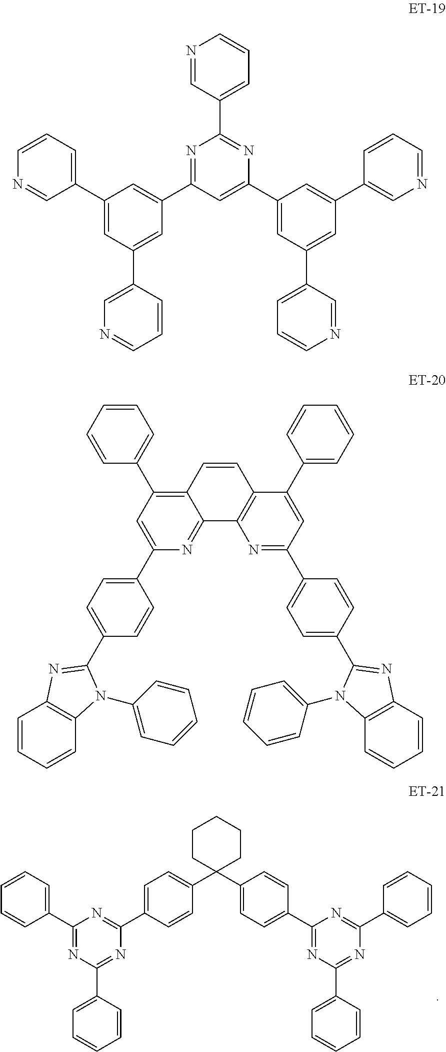 [SMILES: c1cncc(-c2cc(-c3cccnc3)cc(-c3cc(-c4cc(-c5cccnc5)cc(-c5cccnc5)c4)nc(-c4cccnc4)n3)c2)c1 ; c1ccc(-c2cc(-c3ccc(-c4nc5ccccc5n4-c4ccccc4)cc3)nc3c2ccc2c(-c4ccccc4)cc(-c4ccc(-c5nc6ccccc6n5-c5ccccc5)cc4)nc23)cc1 ; c1ccc(-c2nc(-c3ccccc3)nc(-c3ccc(C4(c5ccc(-c6nc(-c7ccccc7)nc(-c7ccccc7)n6)cc5)CCCCC4)cc3)n2)cc1]

In an embodiment, the cathode 200 may comprise a cathode material, which is a low work function material contributing to injection of electrons into the functional layer. Specific examples of the cathode material include, but are not limited to, metals such as magnesium, calcium, sodium, potassium, titanium, indium, yttrium, lithium, gadolinium, aluminum, silver, tin, lead, and alloys thereof; or multilayer materials such as LiF/Al, Liq/Al, LiO₂/Al, LiF/Ca, LiF/Al, and BaF₂/Ca. Preferably, a metal electrode comprising magnesium and silver as the cathode is included.

Optionally, as shown in FIG. 1, a hole injection layer 310 may be further provided between the anode 100 and the first hole transport layer 321 to enhance the ability to inject holes into the first hole transport layer 321. The hole injection layer 310 may choose to use a benzidine derivative, a starburst arylamine-based compound, a phthalocyanine derivative or other materials. It is not particularly defined in the present disclosure. For example, the compound contained in the hole injection layer 310 is selected from the group consisting of the following compounds:

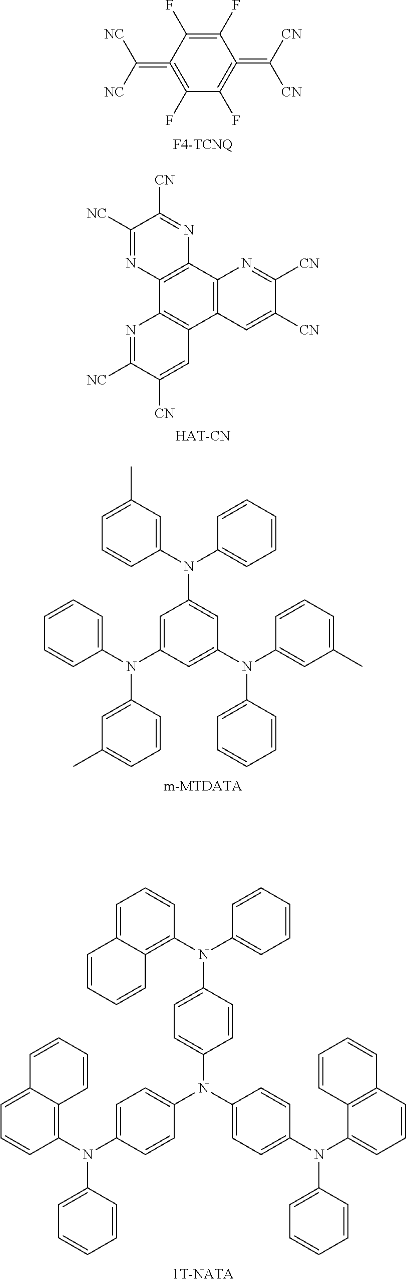

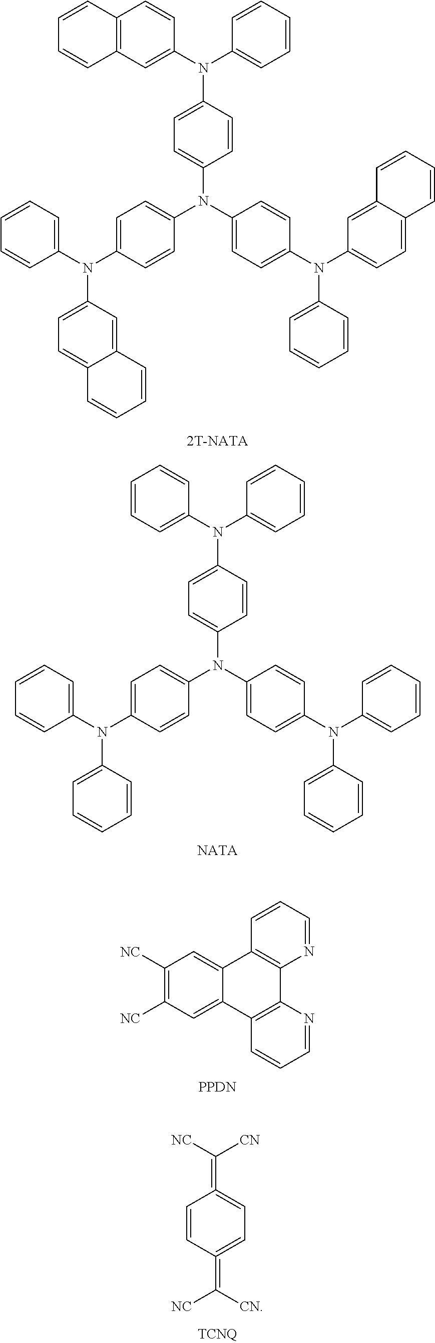

In a specific embodiment of the present disclosure, the hole injection layer 310 is PD and HT-36.

Optionally, as shown in FIG. 1, an electron injection layer 350 is further provided between the cathode 200 and the electron transport layer 340 to enhance the ability to inject electrons into the electron transport layer 340. The electron injection layer 350 may comprise an inorganic material such as an alkali metal sulfide, an alkali metal halide, or may comprise a complex of an alkali metal and an organic compound. For example, the electron injection layer 350 comprises Yb.

According to another embodiment, the electronic element is a photoelectric conversion device. As shown in FIG. 3, the photoelectric conversion device may include an anode 100 and a cathode 200 disposed opposite to each other, and a functional layer 300 disposed between the anode 100 and the cathode 200; the functional layer 300 comprises the organic compounds provided in the present disclosure.

According to a specific embodiment, as shown in FIG. 3, the photoelectric conversion device includes an anode 100, a hole transport layer 320, a photoelectric conversion layer 360, an electron transport layer 340, and a cathode 200 stacked in sequence. Optionally, the hole transport layer 320 comprises the organic compounds of the present disclosure.

Optionally, the photoelectric conversion device may be a solar cell, especially an organic thin film solar cell. For example, in an embodiment of the present disclosure, the solar cell comprises an anode, a hole transport layer, a photoelectric conversion layer, an electron transport layer, and a cathode sequentially stacked, wherein the hole transport layer comprises the organic compound of the present disclosure.

In a third aspect, the present disclosure provides an electronic apparatus, comprising the electronic element provided in the second aspect.

According to an embodiment, as shown in FIG. 2, the electronic apparatus is a first electronic apparatus 400 comprising the above-described organic electroluminescent device. The first electronic apparatus 400 may be, for example, a display apparatus, a lighting apparatus, an optical communication apparatus, or other type of electronic apparatus, examples of which may include, but are not limited to, computer screens, mobile phone screens, televisions, electronic paper, emergency lamps, optical modules, etc.

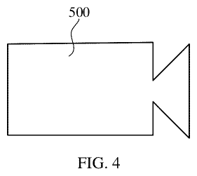

According to another embodiment, as shown in FIG. 4, the electronic apparatus is a second electronic apparatus 500 comprising the above-described organic electroluminescent device. The second electronic apparatus 500 may be, for example, a solar power generation equipment, a photodetector, a fingerprint recognition equipment, an optical module, a CCD camera, or other types of electronic apparatus.

The synthesis method of the organic compound in the present disclosure will be demonstrated in detail with the following synthesis examples, but the present disclosure is not limited in any way by this.

The compounds of the synthetic methods not mentioned in the present disclosure are all raw material products commercially available.

### SYNTHESIS EXAMPLE

### 1. Synthesis of Sub-a1

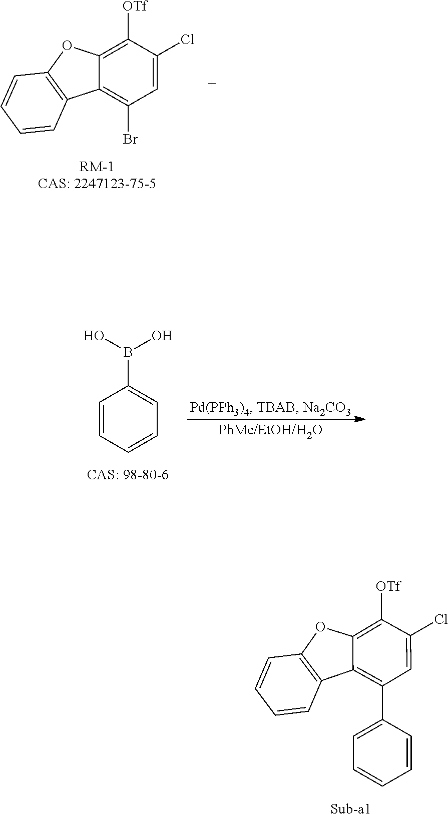 [SMILES: O=S(=O)(Oc1c(Cl)cc(Br)c2c1oc1ccccc12)C(F)(F)F ; OB(O)c1ccccc1 ; O=S(=O)(Oc1c(Cl)cc(-c2ccccc2)c2c1oc1ccccc12)C(F)(F)F]

Under a nitrogen atmosphere, to a 1000 mL three-necked flask was sequentially added RM-1 (21.48 g, 50 mmol), phenylboronic acid (6.70 g, 55 mmol), tetrabutylammonium bromide (1.61 g, 5 mmol), tetrakis(triphenylphosphine) palladium (0.58 g, 0.5 mmol), anhydrous sodium carbonate (10.60 g, 100 mmol), toluene (220 mL), anhydrous ethanol (45 mL), and deionized water (45 mL). Stirring and heating were initiated, and the reaction was heated to reflux for 24 hours of reaction. After the system was cooled down to room temperature, the reaction mixture was extracted with dichloromethane (100 mL×3 times), and the organic phases were combined and dried on anhydrous magnesium sulfate, followed by filtration and then removal of the solvent via distillation under reduced pressure, to obtain a crude product. The crude product was purified by silica gel column chromatography using dichloromethane/n-hexane as the mobile phase, to obtain Sub-a1 as a white solid (15.80 g, yield 74%).

The intermediates Sub-ax listed in Table 1 below were synthesized using the same method as Sub-a1, except that Reactant A was used instead of RM-1 and Reactant B was used instead of phenylboronic acid. The main raw materials used, the synthesized intermediates, and their yields are shown in Table 1.

TABLE 1

| Reactant A | Reactant B | Sub-ax | Yield (%) |
|---|---|---|---|
| 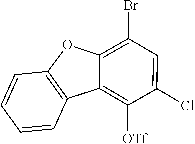 [SMILES: O=S(=O)(Oc1c(Cl)cc(Br)c2oc3ccccc3c12)C(F)(F)F] CAS: 2247123-61-9 | 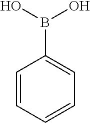 CAS: 98-80-6 | 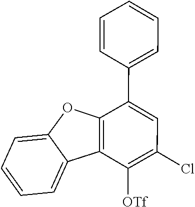 [SMILES: O=S(=O)(Oc1c(Cl)cc(-c2ccccc2)c2oc3ccccc3c12)C(F)(F)F] Sub-a2 | 77 |
| 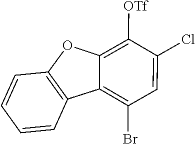 [SMILES: O=S(=O)(Oc1c(Cl)cc(Br)c2c1oc1ccccc12)C(F)(F)F] CAS: 2247123-75-5 | 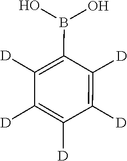 [SMILES: [2H]c1c([2H])c([2H])c(B(O)O)c([2H])c1[2H]] CAS: 215527-70-1 | 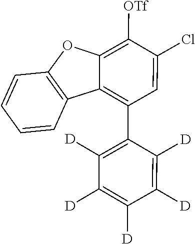 [SMILES: [2H]c1c([2H])c([2H])c(-c2cc(Cl)c(OS(=O)(=O)C(F)(F)F)c3oc4ccccc4c23)c([2H])c1[2H]] Sub-a3 | 68 |
| 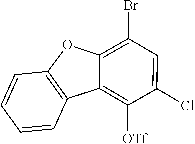 [SMILES: O=S(=O)(Oc1c(Cl)cc(Br)c2oc3ccccc3c12)C(F)(F)F] CAS: 2247123-61-9 | 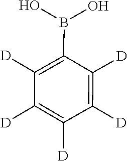 [SMILES: [2H]c1c([2H])c([2H])c(B(O)O)c([2H])c1[2H]] CAS: 215527-70-1 | 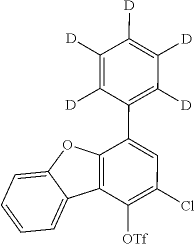 [SMILES: [2H]c1c([2H])c([2H])c(-c2cc(Cl)c(OS(=O)(=O)C(F)(F)F)c3c2oc2ccccc23)c([2H])c1[2H]] Sub-a4 | 72 |

### 2. Synthesis of Sub-b1

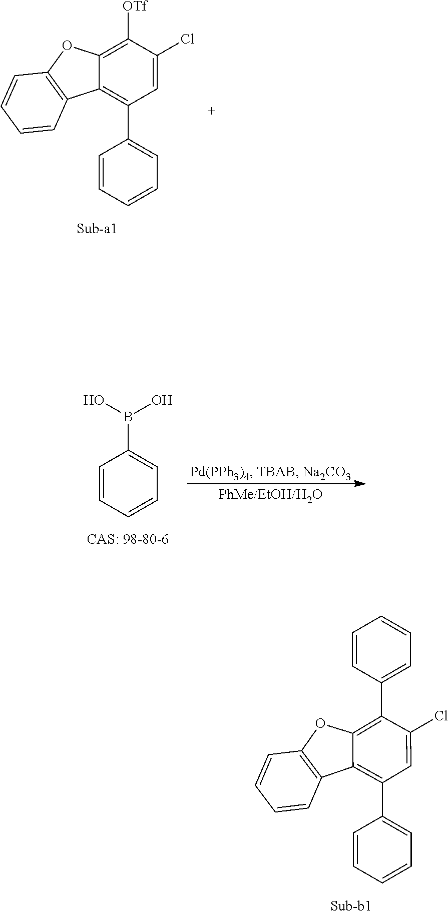 [SMILES: O=S(=O)(Oc1c(Cl)cc(-c2ccccc2)c2c1oc1ccccc12)C(F)(F)F ; OB(O)c1ccccc1 ; Clc1cc(-c2ccccc2)c2c(oc3ccccc32)c1-c1ccccc1]

Under a nitrogen atmosphere, to a 500 mL three-necked flask was sequentially added Sub-a1 (21.34 g, 50 mmol), phenylboronic acid (6.70 g, 55 mmol), tetrakis(triphenylphosphine) palladium (0.58 g, 0.5 mmol), anhydrous potassium carbonate (13.82 g, 100 mmol), toluene (220 mL), tetrahydrofuran (45 mL), and deionized water (45 mL). Stirring and heating were initiated, and the reaction was heated to reflux for 8 hours of reaction. After the system was cooled down to room temperature, the reaction mixture was extracted with dichloromethane (100 mL×3 times), and the organic phases were combined and dried on anhydrous magnesium sulfate, followed by filtration and then removal of the solvent via distillation under reduced pressure, to obtain a crude product. The crude product was purified by silica gel column chromatography using dichloromethane/n-hexane as the mobile phase, to obtain Sub-b1 as a white solid (11.0 g, yield 62%).

The intermediates listed in Table 2 were synthesized using the same method as Sub-b1, except that Reactant C was used instead of Sub-a1 and Reactant D was used instead of phenylboronic acid. The main raw materials used, the synthesized intermediates, and their yields are shown in Table 2.

TABLE 2

| Reactant C | Reactant D | Structure of Sub-bx | Yield (%) |
|---|---|---|---|
| 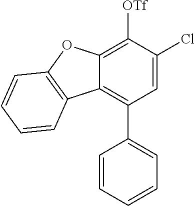 [SMILES: O=S(=O)(Oc1c(Cl)cc(-c2ccccc2)c2c1oc1ccccc12)C(F)(F)F] Sub-a1 | 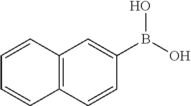 [SMILES: OB(O)c1ccc2ccccc2c1] CAS: 32316-92-0 | 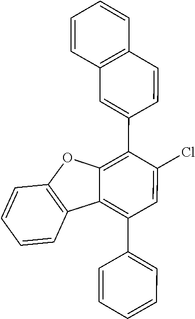 [SMILES: Clc1cc(-c2ccccc2)c2c(oc3ccccc32)c1-c1ccc2ccccc2c1] Sub-b4 | 60 |
| 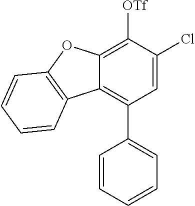 [SMILES: O=S(=O)(Oc1c(Cl)cc(-c2ccccc2)c2c1oc1ccccc12)C(F)(F)F] Sub-a1 | 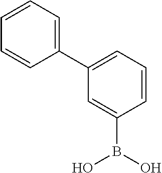 [SMILES: OB(O)c1cccc(-c2ccccc2)c1] CAS: 5122-95-2 | 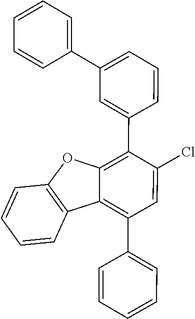 [SMILES: Clc1cc(-c2ccccc2)c2c(oc3ccccc32)c1-c1cccc(-c2ccccc2)c1] Sub-b5 | 57 |
| 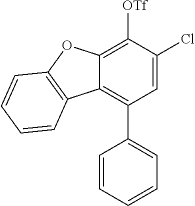 [SMILES: O=S(=O)(Oc1c(Cl)cc(-c2ccccc2)c2c1oc1ccccc12)C(F)(F)F] Sub-a1 | 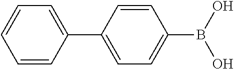 [SMILES: OB(O)c1ccc(-c2ccccc2)cc1] CAS: 5122-94-1 | 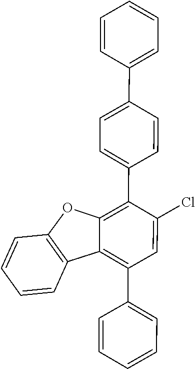 [SMILES: Clc1cc(-c2ccccc2)c2c(oc3ccccc32)c1-c1ccc(-c2ccccc2)cc1] Sub-b7 | 65 |
| 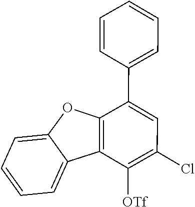 [SMILES: O=S(=O)(Oc1c(Cl)cc(-c2ccccc2)c2oc3ccccc3c12)C(F)(F)F] Sub-a2 | 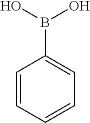 CAS: 98-80-6 | 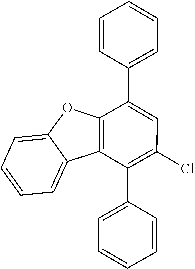 [SMILES: Clc1cc(-c2ccccc2)c2oc3ccccc3c2c1-c1ccccc1] Sub-b2 | 56 |
| 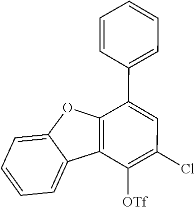 [SMILES: O=S(=O)(Oc1c(Cl)cc(-c2ccccc2)c2oc3ccccc3c12)C(F)(F)F] Sub-a2 | 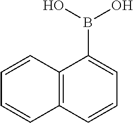 [SMILES: OB(O)c1cccc2ccccc12] CAS: 13922-41-3 | 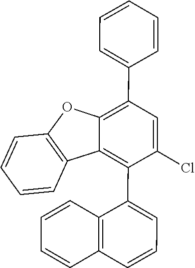 [SMILES: Clc1cc(-c2ccccc2)c2oc3ccccc3c2c1-c1cccc2ccccc12] Sub-b3 | 53 |
| 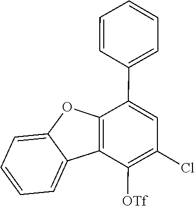 [SMILES: O=S(=O)(Oc1c(Cl)cc(-c2ccccc2)c2oc3ccccc3c12)C(F)(F)F] Sub-a2 | 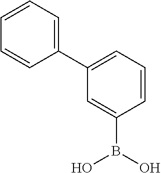 [SMILES: OB(O)c1cccc(-c2ccccc2)c1] CAS: 5122-95-2 | 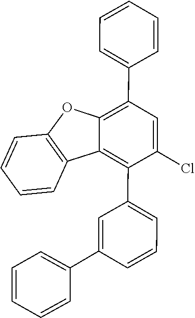 [SMILES: Clc1cc(-c2ccccc2)c2oc3ccccc3c2c1-c1cccc(-c2ccccc2)c1] Sub-b6 | 63 |
| 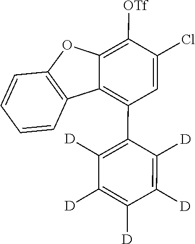 [SMILES: [2H]c1c([2H])c([2H])c(-c2cc(Cl)c(OS(=O)(=O)C(F)(F)F)c3oc4ccccc4c23)c([2H])c1[2H]] | 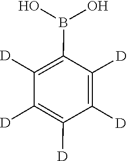 [SMILES: [2H]c1c([2H])c([2H])c(B(O)O)c([2H])c1[2H]] CAS: 215527-70-1 | 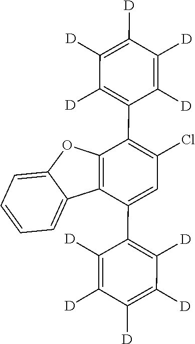 [SMILES: [2H]c1c([2H])c([2H])c(-c2c(Cl)cc(-c3c([2H])c([2H])c([2H])c([2H])c3[2H])c3c2oc2ccccc23)c([2H])c1[2H]] Sub-b8 | 60 |
| 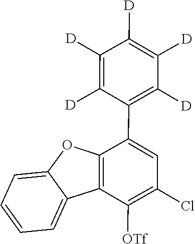 [SMILES: [2H]c1c([2H])c([2H])c(-c2cc(Cl)c(OS(=O)(=O)C(F)(F)F)c3c2oc2ccccc23)c([2H])c1[2H]] | 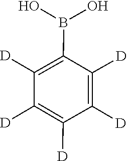 [SMILES: [2H]c1c([2H])c([2H])c(B(O)O)c([2H])c1[2H]] CAS: 215527-70-1 | 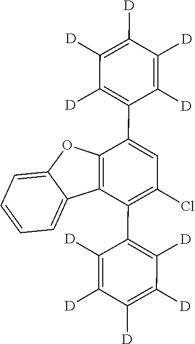 [SMILES: [2H]c1c([2H])c([2H])c(-c2cc(Cl)c(-c3c([2H])c([2H])c([2H])c([2H])c3[2H])c3c2oc2ccccc23)c([2H])c1[2H]] Sub-b9 | 62 |

### 3. Synthesis of Sub-c1

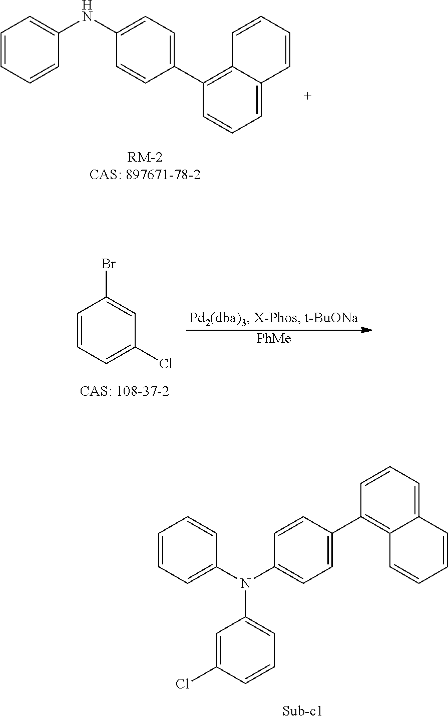 [SMILES: c1ccc(Nc2ccc(-c3cccc4ccccc34)cc2)cc1 ; Clc1cccc(Br)c1 ; "PhMe" ; Clc1cccc(N(c2ccccc2)c2ccc(-c3cccc4ccccc34)cc2)c1]

Under a nitrogen atmosphere, to a 500 mL three necked flash was sequentially added RM-2 (14.77 g, 50 mmol), m-chlorobromobenzene (9.57 g, 50 mmol), tris(dibenzylidene acetone) dipalladium (0.916 g, 1 mmol), 2-dicyclohexylphosphino-2′,4′,6′-triisopropylbiphenyl (0.95 g, 2 mmol), sodium tert-butoxide (9.61 g, 100 mmol), and toluene (150 mL). The mixture was heated to reflux and react overnight with stirring. After the system was cooled down to room temperature, the reaction mixture was extracted with dichloromethane (100 mL×3 times), and the organic phases were combined and dried on anhydrous sodium sulfate, followed by filtration and then removal of the solvent via distillation under reduced pressure, to obtain a crude product. The crude product was purified by silica gel column chromatography using n-hexane/dichloromethane as the mobile phase, to obtain Sub-c1 (15.62 g, yield 77%) as a grayish white solid.

The intermediates listed in Table 3 below were synthesized using the same method as Sub-c1, except that Reactant E was used instead of RM-2 and Reactant F was used instead of m-chlorobromobenzene. The main raw materials used, the synthesized intermediates, and their yields are shown in Table 3.

TABLE 3

| Reactant E | Reactant F | Sub-c structure | Yield (%) |
|---|---|---|---|
| 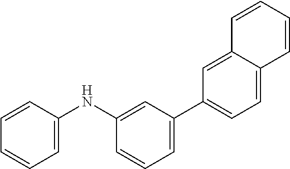 [SMILES: c1ccc(Nc2cccc(-c3ccc4ccccc4c3)c2)cc1] CAS: 1788082-52-9 | 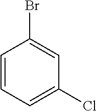 CAS: 108-37-2 | 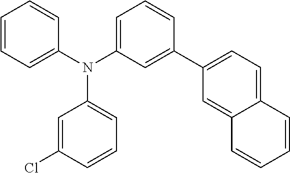 [SMILES: Clc1cccc(N(c2ccccc2)c2cccc(-c3ccc4ccccc4c3)c2)c1] Sub-c2 | 74 |
| 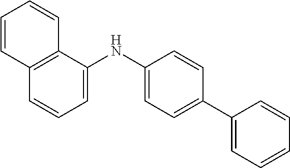 [SMILES: c1ccc(-c2ccc(Nc3cccc4ccccc34)cc2)cc1] CAS: 446242-37-1 | 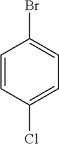 CAS: 106-39-8 | 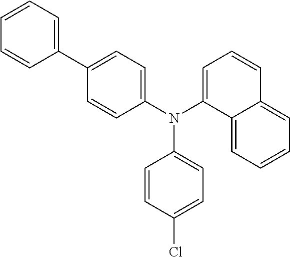 [SMILES: Clc1ccc(N(c2ccc(-c3ccccc3)cc2)c2cccc3ccccc23)cc1] Sub-c3 | 71 |
| 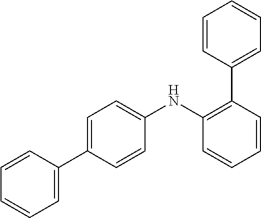 [SMILES: c1ccc(-c2ccc(Nc3ccccc3-c3ccccc3)cc2)cc1] CAS: 1372775-52-4 | 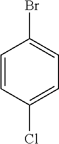 CAS: 106-39-8 | 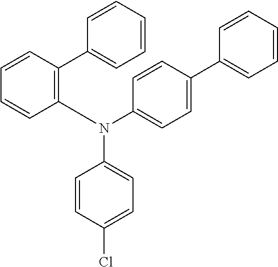 [SMILES: Clc1ccc(N(c2ccc(-c3ccccc3)cc2)c2ccccc2-c2ccccc2)cc1] Sub-c4 | 69 |
| 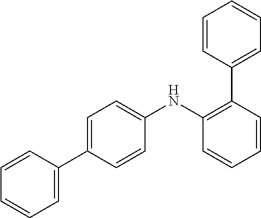 [SMILES: c1ccc(-c2ccc(Nc3ccccc3-c3ccccc3)cc2)cc1] CAS: 1372775-52-4 | 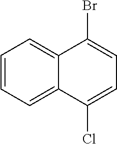 [SMILES: Clc1ccc(Br)c2ccccc12] CAS: 53220-82-9 | 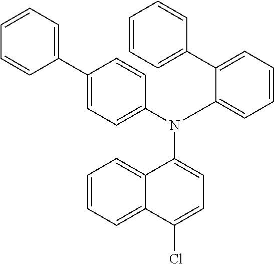 [SMILES: Clc1ccc(N(c2ccc(-c3ccccc3)cc2)c2ccccc2-c2ccccc2)c2ccccc12] Sub-c17 | 61 |
| 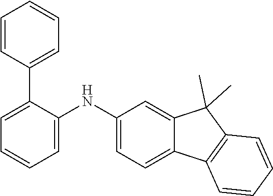 [SMILES: CC1(C)c2ccccc2-c2ccc(Nc3ccccc3-c3ccccc3)cc21] CAS: 1198395-24-2 | 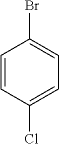 CAS: 106-39-8 | 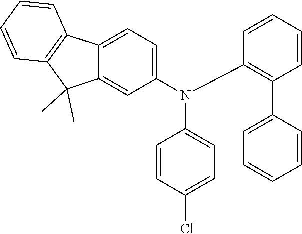 [SMILES: CC1(C)c2ccccc2-c2ccc(N(c3ccc(Cl)cc3)c3ccccc3-c3ccccc3)cc21] Sub-c5 | 67 |
| 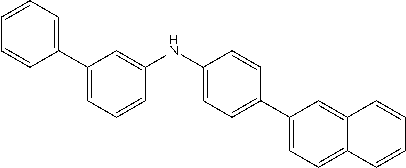 [SMILES: c1ccc(-c2cccc(Nc3ccc(-c4ccc5ccccc5c4)cc3)c2)cc1] CAS: 1446448-97-0 | 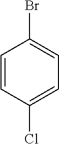 CAS: 106-39-8 | 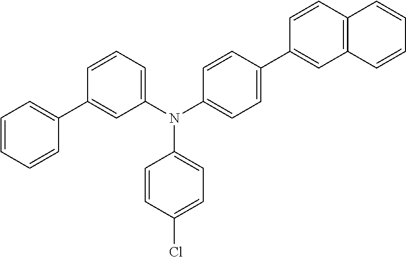 [SMILES: Clc1ccc(N(c2ccc(-c3ccc4ccccc4c3)cc2)c2cccc(-c3ccccc3)c2)cc1] Sub-c6 | 73 |
| 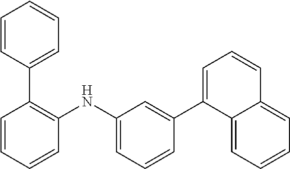 [SMILES: c1ccc(-c2ccccc2Nc2cccc(-c3cccc4ccccc34)c2)cc1] CAS: 2722640-39-1 | 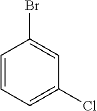 CAS: 108-37-2 | 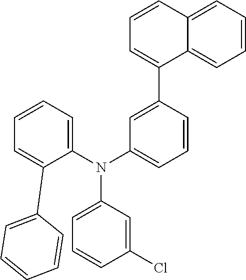 [SMILES: Clc1cccc(N(c2cccc(-c3cccc4ccccc34)c2)c2ccccc2-c2ccccc2)c1] Sub-c7 | 64 |
| 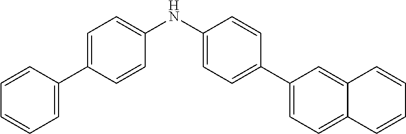 [SMILES: c1ccc(-c2ccc(Nc3ccc(-c4ccc5ccccc5c4)cc3)cc2)cc1] CAS: 897921-60-7 | 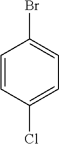 CAS: 106-39-8 | 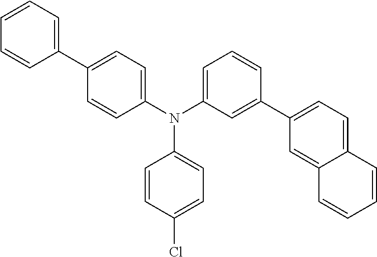 [SMILES: Clc1ccc(N(c2ccc(-c3ccccc3)cc2)c2cccc(-c3ccc4ccccc4c3)c2)cc1] Sub-c8 | 71 |
| 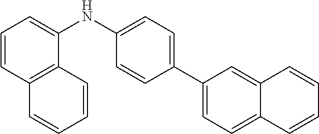 [SMILES: c1ccc2cc(-c3ccc(Nc4cccc5ccccc45)cc3)ccc2c1] CAS: 1400923-26-3 | 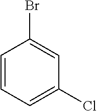 CAS: 108-37-2 | 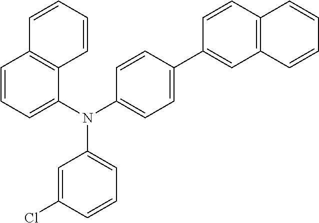 [SMILES: Clc1cccc(N(c2ccc(-c3ccc4ccccc4c3)cc2)c2cccc3ccccc23)c1] Sub-c9 | 74 |
| 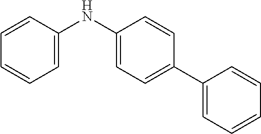 [SMILES: c1ccc(Nc2ccc(-c3ccccc3)cc2)cc1] CAS: 32228-99-2 | 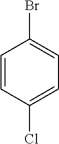 CAS: 106-39-8 | 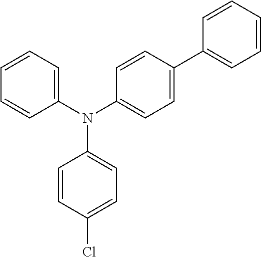 [SMILES: Clc1ccc(N(c2ccccc2)c2ccc(-c3ccccc3)cc2)cc1] Sub-c10 | 78 |
| 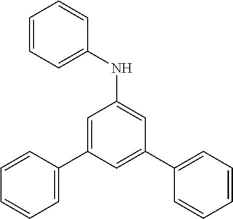 [SMILES: c1ccc(Nc2cc(-c3ccccc3)cc(-c3ccccc3)c2)cc1] CAS: 1065663-41-3 | 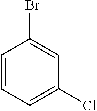 CAS: 108-37-2 | 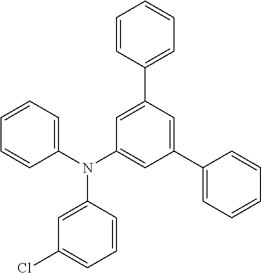 [SMILES: Clc1cccc(N(c2ccccc2)c2cc(-c3ccccc3)cc(-c3ccccc3)c2)c1] Sub-c11 | 74 |
| 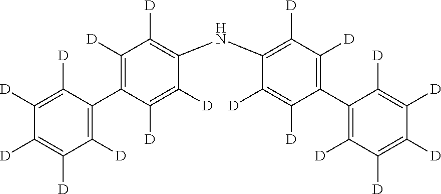 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] CAS: 1879963-55-9 | 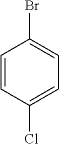 CAS: 106-39-8 | 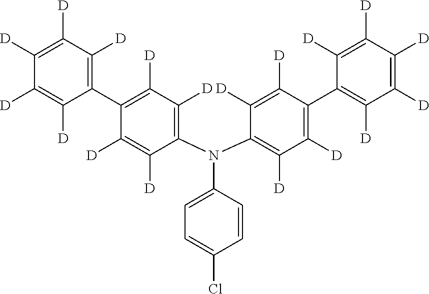 [SMILES: [2H]c1c([2H])c([2H])c(-c2c([2H])c([2H])c(N(c3ccc(Cl)cc3)c3c([2H])c([2H])c(-c4c([2H])c([2H])c([2H])c([2H])c4[2H])c([2H])c3[2H])c([2H])c2[2H])c([2H])c1[2H]] Sub-c12 | 71 |
| 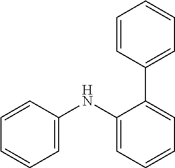 [SMILES: c1ccc(Nc2ccccc2-c2ccccc2)cc1] CAS: 35887-50-4 | 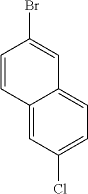 [SMILES: Clc1ccc2cc(Br)ccc2c1] CAS: 870822-84-7 | 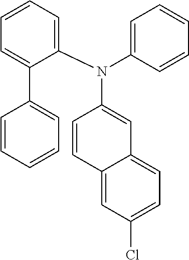 [SMILES: Clc1ccc2cc(N(c3ccccc3)c3ccccc3-c3ccccc3)ccc2c1] Sub-c13 | 70 |
| 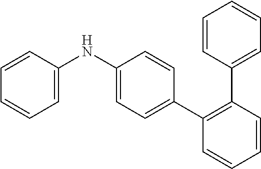 [SMILES: c1ccc(Nc2ccc(-c3ccccc3-c3ccccc3)cc2)cc1] CAS: 1222634-02-7 | 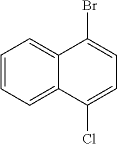 [SMILES: Clc1ccc(Br)c2ccccc12] CAS: 53220-82-9 | 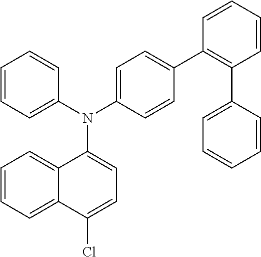 [SMILES: Clc1ccc(N(c2ccccc2)c2ccc(-c3ccccc3-c3ccccc3)cc2)c2ccccc12] Sub-c14 | 73 |
| 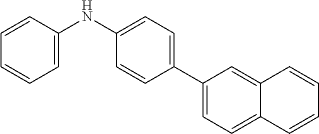 [SMILES: c1ccc(Nc2ccc(-c3ccc4ccccc4c3)cc2)cc1] CAS: 897671-79-3 | 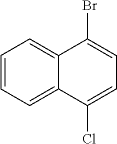 [SMILES: Clc1ccc(Br)c2ccccc12] CAS: 53220-82-9 | 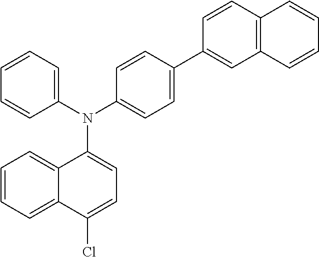 [SMILES: Clc1ccc(N(c2ccccc2)c2ccc(-c3ccc4ccccc4c3)cc2)c2ccccc12] Sub-c15 | 70 |
| 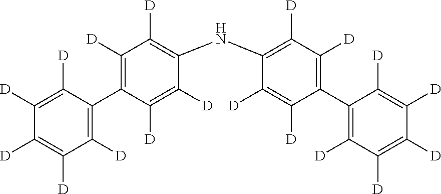 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] CAS: 1879963-55-9 | 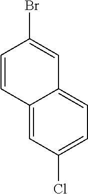 [SMILES: Clc1ccc2cc(Br)ccc2c1] CAS: 870822-84-7 | 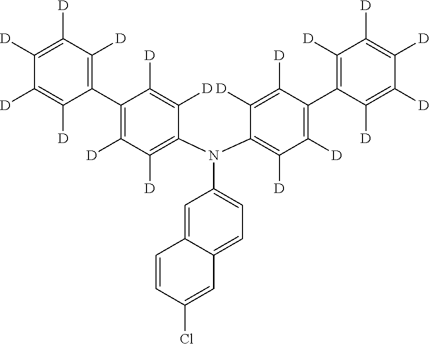 [SMILES: [2H]c1c([2H])c([2H])c(-c2c([2H])c([2H])c(N(c3ccc4cc(Cl)ccc4c3)c3c([2H])c([2H])c(-c4c([2H])c([2H])c([2H])c([2H])c4[2H])c([2H])c3[2H])c([2H])c2[2H])c([2H])c1[2H]] Sub-c16 | 76 |
| 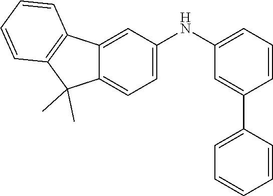 [SMILES: CC1(C)c2ccccc2-c2cc(Nc3cccc(-c4ccccc4)c3)ccc21] CAS: 1613331-97-7 | 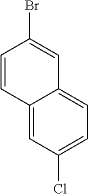 [SMILES: Clc1ccc2cc(Br)ccc2c1] CAS: 870822-84-7 | 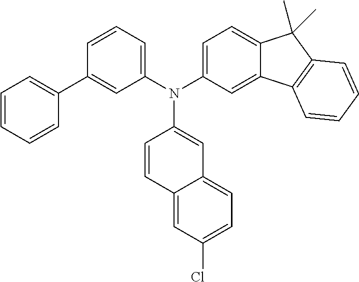 [SMILES: CC1(C)c2ccccc2-c2cc(N(c3cccc(-c4ccccc4)c3)c3ccc4cc(Cl)ccc4c3)ccc21] Sub-c18 | 75 |
| 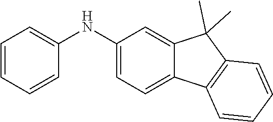 [SMILES: CC1(C)c2ccccc2-c2ccc(Nc3ccccc3)cc21] CAS: 355832-04-1 | 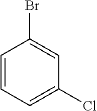 CAS: 108-37-2 | 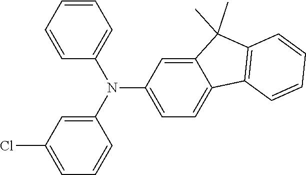 [SMILES: CC1(C)c2ccccc2-c2ccc(N(c3ccccc3)c3cccc(Cl)c3)cc21] Sub-c19 | 72 |
| 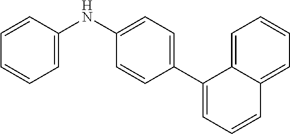 [SMILES: c1ccc(Nc2ccc(-c3cccc4ccccc34)cc2)cc1] CAS: 897671-78-2 | 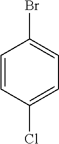 CAS: 106-39-8 | 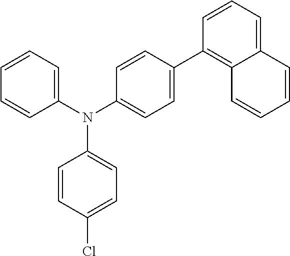 [SMILES: Clc1ccc(N(c2ccccc2)c2ccc(-c3cccc4ccccc34)cc2)cc1] Sub-c20 | 72 |

### 4. Synthesis of Sub-d1

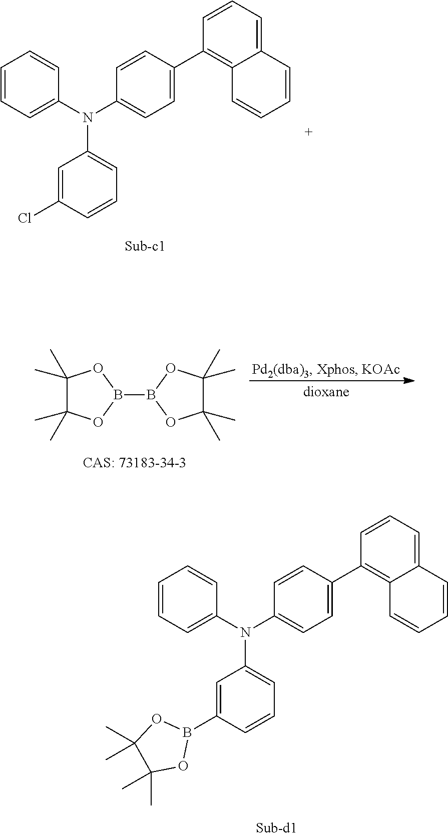

Under a nitrogen atmosphere, to a 500 mL three-necked flask was added Sub-c1 (20.30 g, 50 mmol), biboronic acid pinacol ester (14.0 g, 55 mmol), potassium acetate (10.8 g, 110 mmol) and 1,4-dioxane (160 mL). Stirring and heating were initiated, and tris (dibenzylidene acetone) dipalladium (0.46 g, 0.50 mmol) and (2-dicyclohexylphosphino-2′,4′,6′-triisopropylbiphenyl) (0.48 g, 1.0 mmol) were quickly added when the resulting mixture was heated to 40° C. The resulting mixture was continued to be heated to reflux and react overnight with stirring. After the reaction solution was cooled to room temperature, 200 mL water was added, with sufficiently stirring for 30 minutes. The resulting solution was suction filtered under reduced pressure, and the filter cake was washed with deionized water until neutral, and then rinsed with 100 mL anhydrous ethanol, to obtain a gray solid. The crude product was beaten with n-heptane once, and then is dissolved in 200 mL toluene, followed by passing through a silica gel column to remove the catalyst, to obtain Sub-d1 (15.67 g, yield 63%) as a white solid after concentration.

The intermediates listed in Table 4 below were synthesized using the same method as Sub-d1, except that Reactant G was used instead of Sub-d1. The main raw materials used, the synthesized intermediates, and their yields are shown in Table 4.

TABLE 4

| Reactant G | Sub-d structure | Yield (%) |
|---|---|---|
| 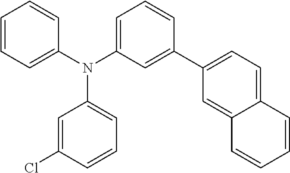 [SMILES: Clc1cccc(N(c2ccccc2)c2cccc(-c3ccc4ccccc4c3)c2)c1] Sub-c2 | 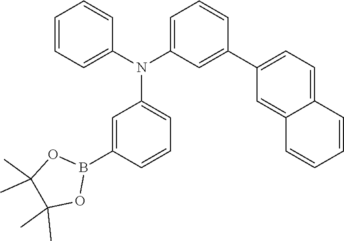 [SMILES: CC1(C)OB(c2cccc(N(c3ccccc3)c3cccc(-c4ccc5ccccc5c4)c3)c2)OC1(C)C] Sub-d2 | 55 |
| 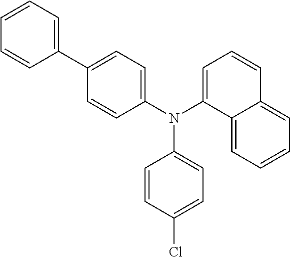 [SMILES: Clc1ccc(N(c2ccc(-c3ccccc3)cc2)c2cccc3ccccc23)cc1] Sub-c3 | 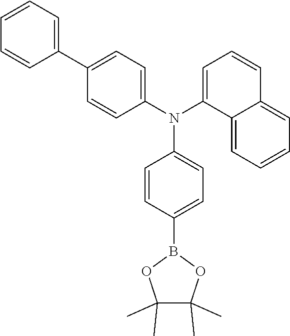 [SMILES: CC1(C)OB(c2ccc(N(c3ccc(-c4ccccc4)cc3)c3cccc4ccccc34)cc2)OC1(C)C] Sub-d3 | 58 |
| 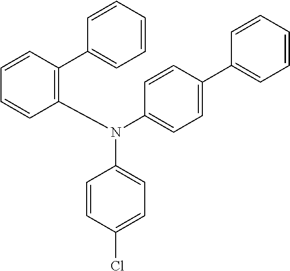 [SMILES: Clc1ccc(N(c2ccc(-c3ccccc3)cc2)c2ccccc2-c2ccccc2)cc1] Sub-c4 | 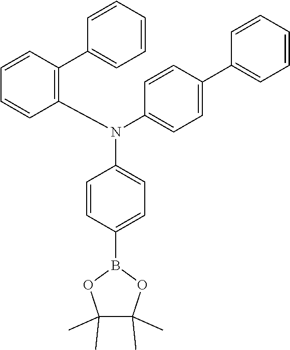 [SMILES: CC1(C)OB(c2ccc(N(c3ccc(-c4ccccc4)cc3)c3ccccc3-c3ccccc3)cc2)OC1(C)C] Sub-d4 | 56 |
| 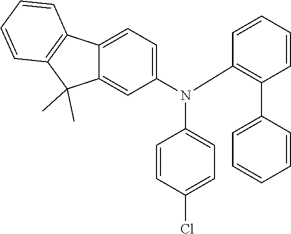 [SMILES: CC1(C)c2ccccc2-c2ccc(N(c3ccc(Cl)cc3)c3ccccc3-c3ccccc3)cc21] Sub-c5 | 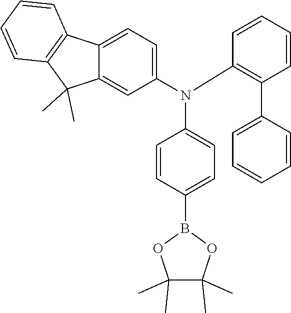 [SMILES: CC1(C)c2ccccc2-c2ccc(N(c3ccc(B4OC(C)(C)C(C)(C)O4)cc3)c3ccccc3-c3ccccc3)cc21] Sub-d5 | 64 |
| 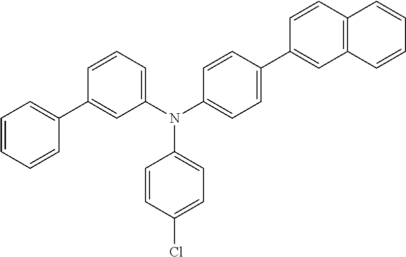 [SMILES: Clc1ccc(N(c2ccc(-c3ccc4ccccc4c3)cc2)c2cccc(-c3ccccc3)c2)cc1] Sub-c6 | 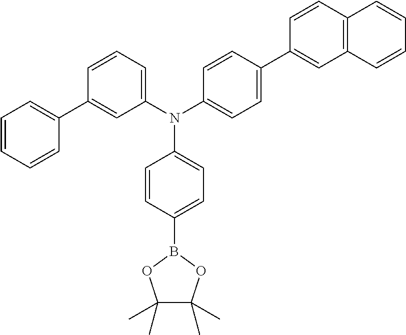 [SMILES: CC1(C)OB(c2ccc(N(c3ccc(-c4ccc5ccccc5c4)cc3)c3cccc(-c4ccccc4)c3)cc2)OC1(C)C] Sub-d6 | 59 |
| 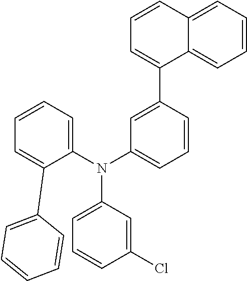 [SMILES: Clc1cccc(N(c2cccc(-c3cccc4ccccc34)c2)c2ccccc2-c2ccccc2)c1] Sub-c7 | 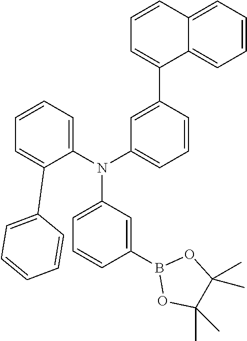 [SMILES: CC1(C)OB(c2cccc(N(c3cccc(-c4cccc5ccccc45)c3)c3ccccc3-c3ccccc3)c2)OC1(C)C] Sub-d7 | 63 |
| 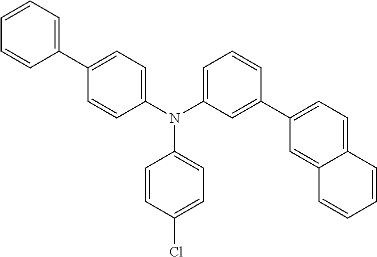 [SMILES: Clc1ccc(N(c2ccc(-c3ccccc3)cc2)c2cccc(-c3ccc4ccccc4c3)c2)cc1] Sub-c8 | 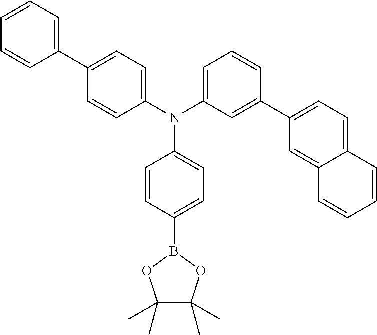 [SMILES: CC1(C)OB(c2ccc(N(c3ccc(-c4ccccc4)cc3)c3cccc(-c4ccc5ccccc5c4)c3)cc2)OC1(C)C] Sub-d8 | 64 |
| 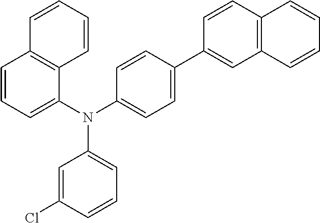 [SMILES: Clc1cccc(N(c2ccc(-c3ccc4ccccc4c3)cc2)c2cccc3ccccc23)c1] Sub-c9 | 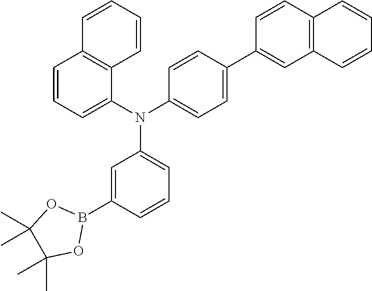 [SMILES: CC1(C)OB(c2cccc(N(c3ccc(-c4ccc5ccccc5c4)cc3)c3cccc4ccccc34)c2)OC1(C)C] Sub-d9 | 56 |
| 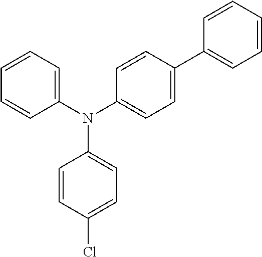 [SMILES: Clc1ccc(N(c2ccccc2)c2ccc(-c3ccccc3)cc2)cc1] Sub-c10 | 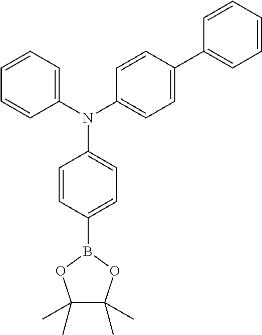 [SMILES: CC1(C)OB(c2ccc(N(c3ccccc3)c3ccc(-c4ccccc4)cc3)cc2)OC1(C)C] Sub-d10 | 63 |
| 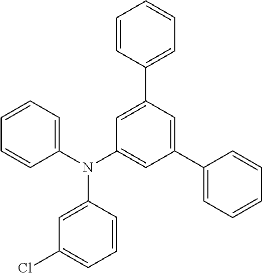 [SMILES: Clc1cccc(N(c2ccccc2)c2cc(-c3ccccc3)cc(-c3ccccc3)c2)c1] Sub-c11 | 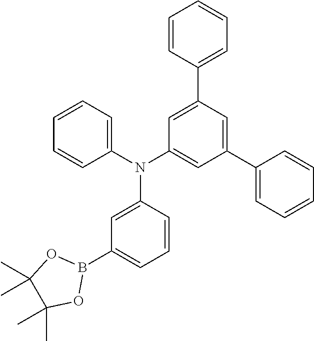 [SMILES: CC1(C)OB(c2cccc(N(c3ccccc3)c3cc(-c4ccccc4)cc(-c4ccccc4)c3)c2)OC1(C)C] Sub-d11 | 63 |
| 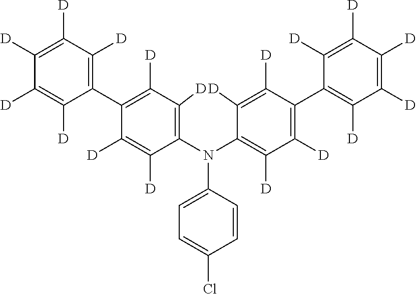 [SMILES: [2H]c1c([2H])c([2H])c(-c2c([2H])c([2H])c(N(c3ccc(Cl)cc3)c3c([2H])c([2H])c(-c4c([2H])c([2H])c([2H])c([2H])c4[2H])c([2H])c3[2H])c([2H])c2[2H])c([2H])c1[2H]] Sub-c12 | 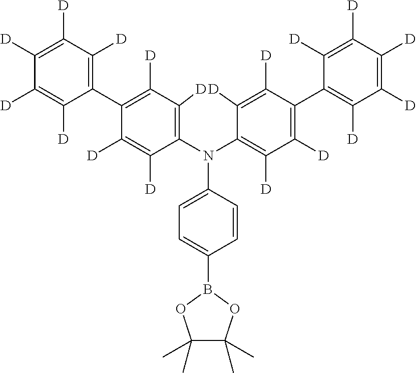 [SMILES: [2H]c1c([2H])c([2H])c(-c2c([2H])c([2H])c(N(c3ccc(B4OC(C)(C)C(C)(C)O4)cc3)c3c([2H])c([2H])c(-c4c([2H])c([2H])c([2H])c([2H])c4[2H])c([2H])c3[2H])c([2H])c2[2H])c([2H])c1[2H]] Sub-d12 | 65 |
| 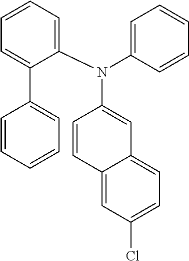 [SMILES: Clc1ccc2cc(N(c3ccccc3)c3ccccc3-c3ccccc3)ccc2c1] Sub-c13 | 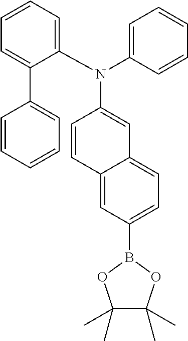 [SMILES: CC1(C)OB(c2ccc3cc(N(c4ccccc4)c4ccccc4-c4ccccc4)ccc3c2)OC1(C)C] Sub-d13 | 63 |
| 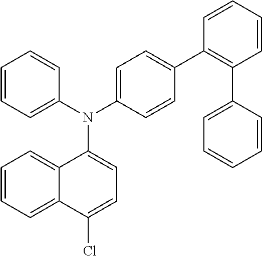 [SMILES: Clc1ccc(N(c2ccccc2)c2ccc(-c3ccccc3-c3ccccc3)cc2)c2ccccc12] Sub-c14 | 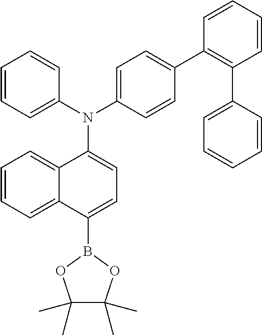 [SMILES: CC1(C)OB(c2ccc(N(c3ccccc3)c3ccc(-c4ccccc4-c4ccccc4)cc3)c3ccccc23)OC1(C)C] Sub-d14 | 56 |
| 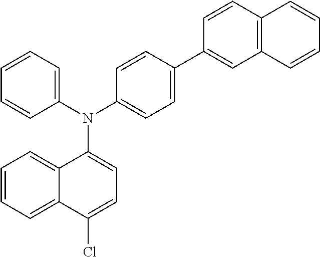 [SMILES: Clc1ccc(N(c2ccccc2)c2ccc(-c3ccc4ccccc4c3)cc2)c2ccccc12] Sub-c15 | 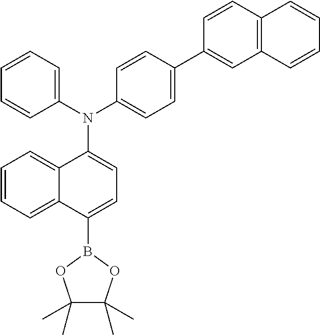 [SMILES: CC1(C)OB(c2ccc(N(c3ccccc3)c3ccc(-c4ccc5ccccc5c4)cc3)c3ccccc23)OC1(C)C] Sub-d15 | 65 |
| 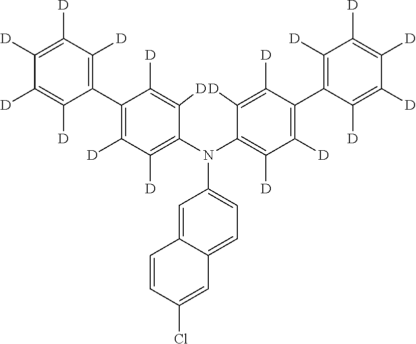 [SMILES: [2H]c1c([2H])c([2H])c(-c2c([2H])c([2H])c(N(c3ccc4cc(Cl)ccc4c3)c3c([2H])c([2H])c(-c4c([2H])c([2H])c([2H])c([2H])c4[2H])c([2H])c3[2H])c([2H])c2[2H])c([2H])c1[2H]] Sub-c16 | 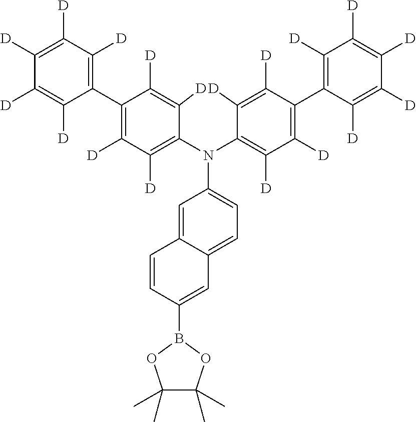 [SMILES: [2H]c1c([2H])c([2H])c(-c2c([2H])c([2H])c(N(c3ccc4cc(B5OC(C)(C)C(C)(C)O5)ccc4c3)c3c([2H])c([2H])c(-c4c([2H])c([2H])c([2H])c([2H])c4[2H])c([2H])c3[2H])c([2H])c2[2H])c([2H])c1[2H]] Sub-d16 | 62 |
| 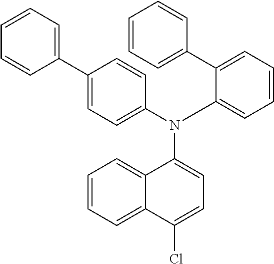 [SMILES: Clc1ccc(N(c2ccc(-c3ccccc3)cc2)c2ccccc2-c2ccccc2)c2ccccc12] Sub-c17 | 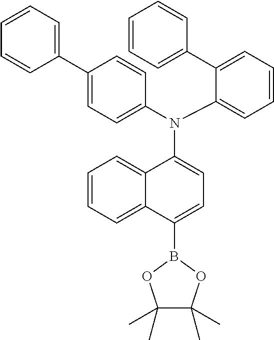 [SMILES: CC1(C)OB(c2ccc(N(c3ccc(-c4ccccc4)cc3)c3ccccc3-c3ccccc3)c3ccccc23)OC1(C)C] Sub-d17 | 62 |
| 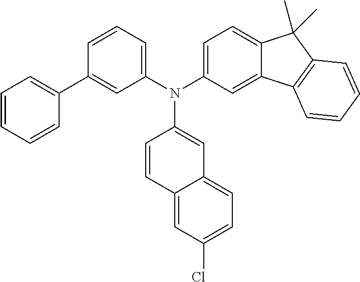 [SMILES: CC1(C)c2ccccc2-c2cc(N(c3cccc(-c4ccccc4)c3)c3ccc4cc(Cl)ccc4c3)ccc21] Sub-c18 | 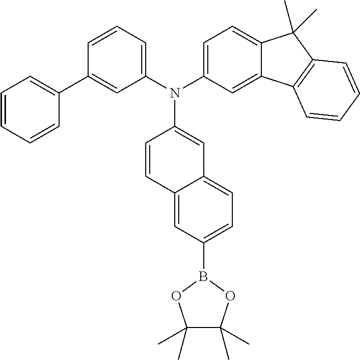 [SMILES: CC1(C)c2ccccc2-c2cc(N(c3cccc(-c4ccccc4)c3)c3ccc4cc(B5OC(C)(C)C(C)(C)O5)ccc4c3)ccc21] Sub-d18 | 60 |
| 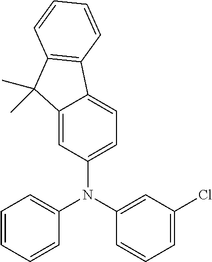 [SMILES: CC1(C)c2ccccc2-c2ccc(N(c3ccccc3)c3cccc(Cl)c3)cc21] Sub-c19 | 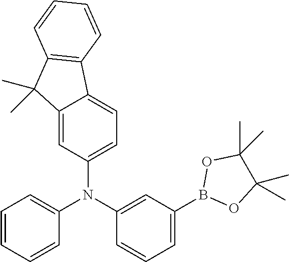 [SMILES: CC1(C)c2ccccc2-c2ccc(N(c3ccccc3)c3cccc(B4OC(C)(C)C(C)(C)O4)c3)cc21] Sub-d19 | 55 |
| 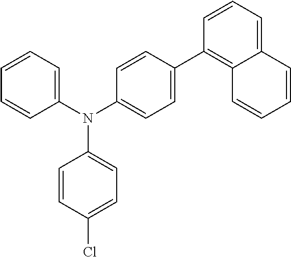 [SMILES: Clc1ccc(N(c2ccccc2)c2ccc(-c3cccc4ccccc34)cc2)cc1] Sub-c20 | 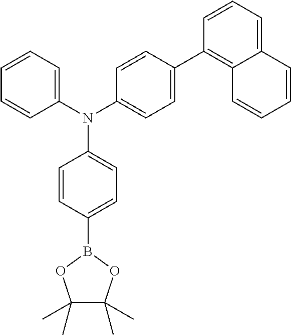 [SMILES: CC1(C)OB(c2ccc(N(c3ccccc3)c3ccc(-c4cccc5ccccc45)cc3)cc2)OC1(C)C] Sub-d20 | 65 |

### 5. Synthesis of Compound 11

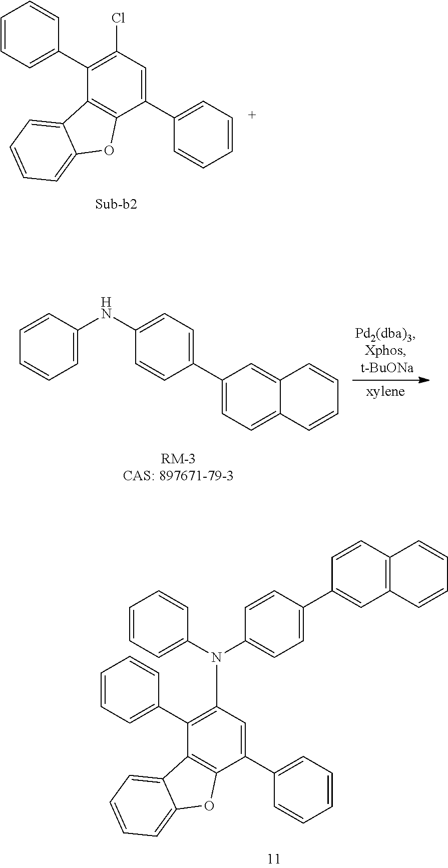 [SMILES: Clc1cc(-c2ccccc2)c2oc3ccccc3c2c1-c1ccccc1 ; c1ccc(Nc2ccc(-c3ccc4ccccc4c3)cc2)cc1 ; c1ccc(-c2cc(N(c3ccccc3)c3ccc(-c4ccc5ccccc5c4)cc3)c(-c3ccccc3)c3c2oc2ccccc23)cc1]

Under a nitrogen atmosphere, to a 250 mL three-necked flask was sequentially added Sub-b2 (8.87 g, 25 mmol), RM-3 (8.12 g, 27.5 mmol), tris(dibenzylidene acetone) dipalladium (0.46 g, 0.5 mmol), 2-dicyclohexylphosphino-2′,4′,6′-triisopropylbiphenyl (0.48 g, 1 mmol), sodium tert-butoxide (9.61 g, 50 mmol) and xylene (100 mL). The resulting mixture was heated to reflux and react overnight with stirring. After the reaction solution was cooled to room temperature, it was extracted with dichloromethane (100 mL×3 times), and the organic phases were combined and dried on anhydrous magnesium sulfate, followed by filtration and then removal of the solvent via distillation under reduced pressure, to obtain a crude product. The crude product was purified by silica gel column chromatography using dichloromethane/n-hexane as the mobile phase, to obtain Compound 11 as a white solid (8.28 g, yield 54%), Mass Spectra (m/z)=614.24 [M+H]⁺.

The compounds listed in Table 5 below were synthesized using the same method as Compound 11, except that Reactant H was used instead of Sub-b2 and Reactant J was used instead of RM-3. The main raw materials used, the synthesized intermediates, and their Mass Spectra and yields are shown in Table 5.

TABLE 5

| | | | Mass | |
|---|---|---|---|---|
| | | | Spectra | |
| | | | (m/z)/ | Yield |
| Reactant H | Reactant J | Compound | [M + H]⁺ | (%) |
| 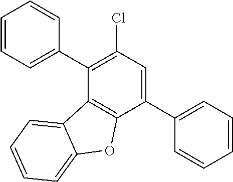 [SMILES: Clc1cc(-c2ccccc2)c2oc3ccccc3c2c1-c1ccccc1] Sub-b2 | 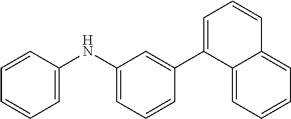 [SMILES: c1ccc(Nc2cccc(-c3cccc4ccccc34)c2)cc1] CAS: 1222634-07-2 | 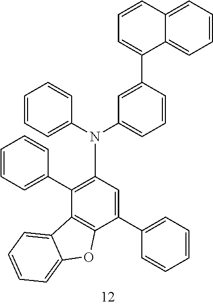 [SMILES: c1ccc(-c2cc(N(c3ccccc3)c3cccc(-c4cccc5ccccc45)c3)c(-c3ccccc3)c3c2oc2ccccc23)cc1] | 614.24 | 54 |
| 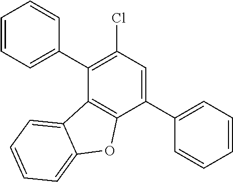 [SMILES: Clc1cc(-c2ccccc2)c2oc3ccccc3c2c1-c1ccccc1] Sub-b2 | 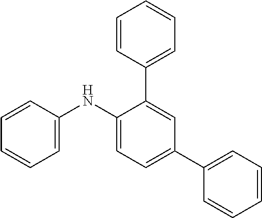 [SMILES: c1ccc(Nc2ccc(-c3ccccc3)cc2-c2ccccc2)cc1] CAS: 1417334-01-0 | 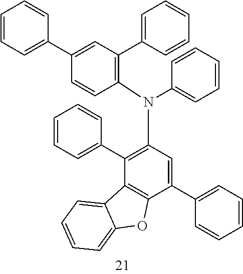 [SMILES: c1ccc(-c2ccc(N(c3ccccc3)c3cc(-c4ccccc4)c4oc5ccccc5c4c3-c3ccccc3)c(-c3ccccc3)c2)cc1] | 640.26 | 47 |
| 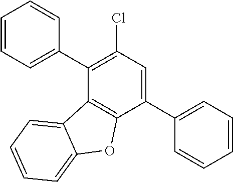 [SMILES: Clc1cc(-c2ccccc2)c2oc3ccccc3c2c1-c1ccccc1] Sub-b2 | 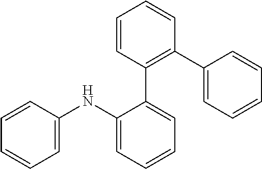 [SMILES: c1ccc(Nc2ccccc2-c2ccccc2-c2ccccc2)cc1] CAS: 1776936-11-8 | 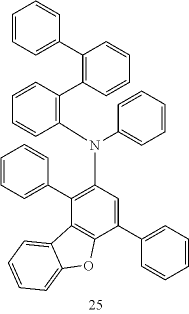 [SMILES: c1ccc(-c2ccccc2-c2ccccc2N(c2ccccc2)c2cc(-c3ccccc3)c3oc4ccccc4c3c2-c2ccccc2)cc1] | 640.26 | 47 |
| 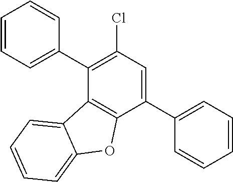 [SMILES: Clc1cc(-c2ccccc2)c2oc3ccccc3c2c1-c1ccccc1] Sub-b2 | 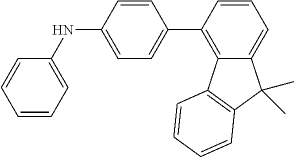 [SMILES: CC1(C)c2ccccc2-c2c(-c3ccc(Nc4ccccc4)cc3)cccc21] CAS: 2640372-62-7 | 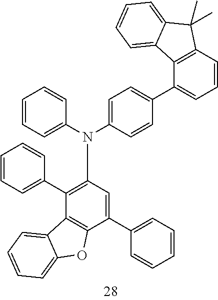 | 680.30 | 52 |
| 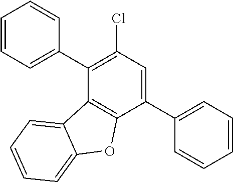 [SMILES: Clc1cc(-c2ccccc2)c2oc3ccccc3c2c1-c1ccccc1] Sub-b2 | 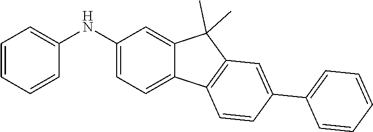 [SMILES: CC1(C)c2cc(Nc3ccccc3)ccc2-c2ccc(-c3ccccc3)cc21] CAS: 1222633-97-7 | 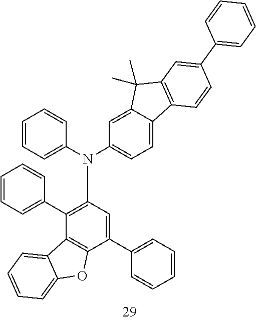 [SMILES: CC1(C)c2cc(-c3ccccc3)ccc2-c2ccc(N(c3ccccc3)c3cc(-c4ccccc4)c4oc5ccccc5c4c3-c3ccccc3)cc21] | 680.30 | 47 |
| 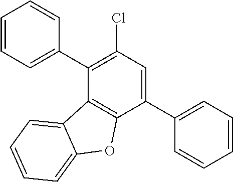 [SMILES: Clc1cc(-c2ccccc2)c2oc3ccccc3c2c1-c1ccccc1] Sub-b2 | 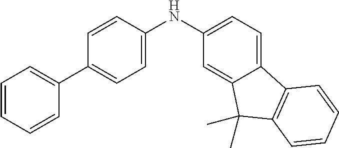 [SMILES: CC1(C)c2ccccc2-c2ccc(Nc3ccc(-c4ccccc4)cc3)cc21] CAS: 897671-69-1 | 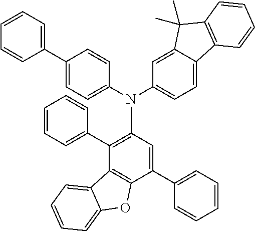 [SMILES: CC1(C)c2ccccc2-c2ccc(N(c3ccc(-c4ccccc4)cc3)c3cc(-c4ccccc4)c4oc5ccccc5c4c3-c3ccccc3)cc21] 37 | 680.30 | 52 |
| 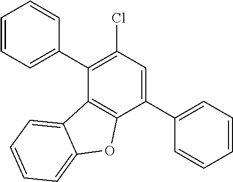 [SMILES: Clc1cc(-c2ccccc2)c2oc3ccccc3c2c1-c1ccccc1] Sub-b2 | 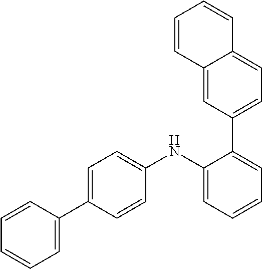 [SMILES: c1ccc(-c2ccc(Nc3ccccc3-c3ccc4ccccc4c3)cc2)cc1] CAS: 2784692-81-3 | 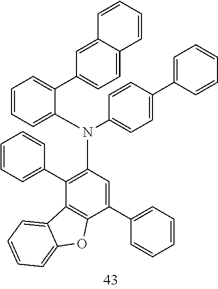 [SMILES: c1ccc(-c2ccc(N(c3ccccc3-c3ccc4ccccc4c3)c3cc(-c4ccccc4)c4oc5ccccc5c4c3-c3ccccc3)cc2)cc1] | 690.27 | 51 |
| 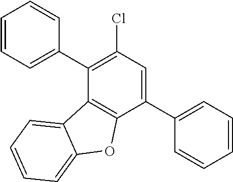 [SMILES: Clc1cc(-c2ccccc2)c2oc3ccccc3c2c1-c1ccccc1] Sub-b2 | 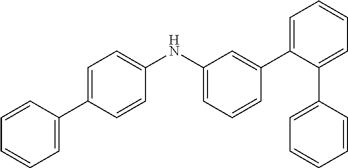 [SMILES: c1ccc(-c2ccc(Nc3cccc(-c4ccccc4-c4ccccc4)c3)cc2)cc1] CAS: 2762148-19-4 | 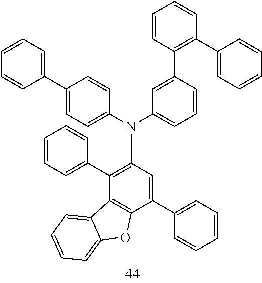 [SMILES: c1ccc(-c2ccc(N(c3cccc(-c4ccccc4-c4ccccc4)c3)c3cc(-c4ccccc4)c4oc5ccccc5c4c3-c3ccccc3)cc2)cc1] | 716.29 | 54 |
| 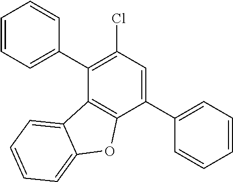 [SMILES: Clc1cc(-c2ccccc2)c2oc3ccccc3c2c1-c1ccccc1] Sub-b2 | 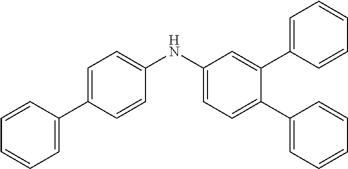 [SMILES: c1ccc(-c2ccc(Nc3ccc(-c4ccccc4)c(-c4ccccc4)c3)cc2)cc1] CAS: 1372775-58-0 | 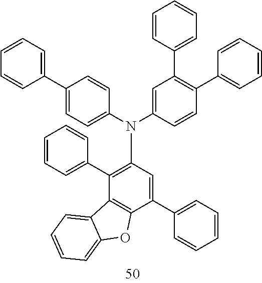 [SMILES: c1ccc(-c2ccc(N(c3ccc(-c4ccccc4)c(-c4ccccc4)c3)c3cc(-c4ccccc4)c4oc5ccccc5c4c3-c3ccccc3)cc2)cc1] | 716.29 | 50 |
| 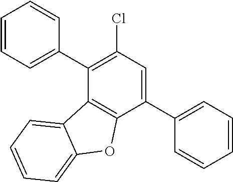 [SMILES: Clc1cc(-c2ccccc2)c2oc3ccccc3c2c1-c1ccccc1] Sub-b2 | 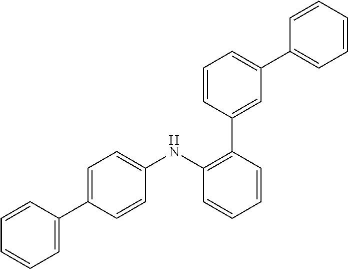 [SMILES: c1ccc(-c2ccc(Nc3ccccc3-c3cccc(-c4ccccc4)c3)cc2)cc1] CAS: 2843596-45-0 | 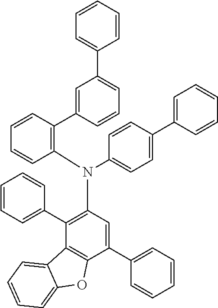 [SMILES: c1ccc(-c2ccc(N(c3ccccc3-c3cccc(-c4ccccc4)c3)c3cc(-c4ccccc4)c4oc5ccccc5c4c3-c3ccccc3)cc2)cc1] 52 | 716.29 | 48 |
| 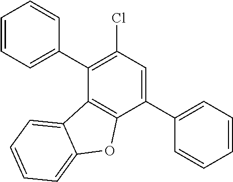 [SMILES: Clc1cc(-c2ccccc2)c2oc3ccccc3c2c1-c1ccccc1] Sub-b2 | 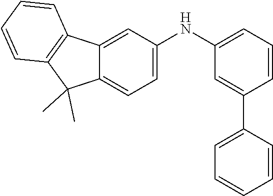 [SMILES: CC1(C)c2ccccc2-c2cc(Nc3cccc(-c4ccccc4)c3)ccc21] CAS: 1613331-97-7 | 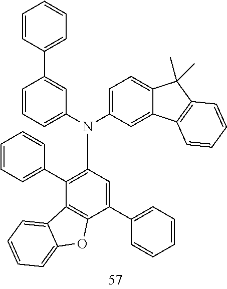 [SMILES: CC1(C)c2ccccc2-c2cc(N(c3cccc(-c4ccccc4)c3)c3cc(-c4ccccc4)c4oc5ccccc5c4c3-c3ccccc3)ccc21] | 680.30 | 46 |
| 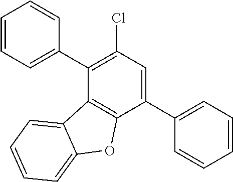 [SMILES: Clc1cc(-c2ccccc2)c2oc3ccccc3c2c1-c1ccccc1] Sub-b2 | 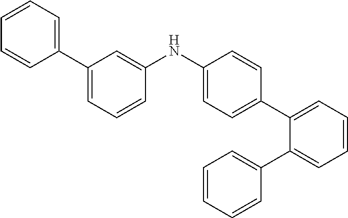 [SMILES: c1ccc(-c2cccc(Nc3ccc(-c4ccccc4-c4ccccc4)cc3)c2)cc1] CAS: 2754302-18-4 | 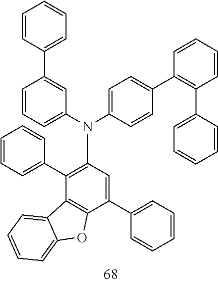 [SMILES: c1ccc(-c2cccc(N(c3ccc(-c4ccccc4-c4ccccc4)cc3)c3cc(-c4ccccc4)c4oc5ccccc5c4c3-c3ccccc3)c2)cc1] | 716.29 | 48 |
| 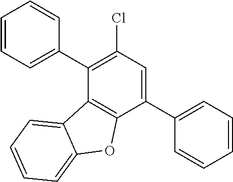 [SMILES: Clc1cc(-c2ccccc2)c2oc3ccccc3c2c1-c1ccccc1] Sub-b2 | 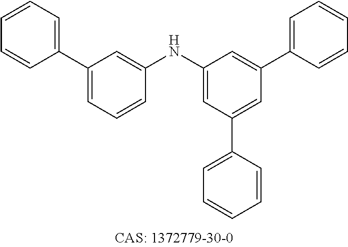 [SMILES: c1ccc(-c2cccc(Nc3cc(-c4ccccc4)cc(-c4ccccc4)c3)c2)cc1] | 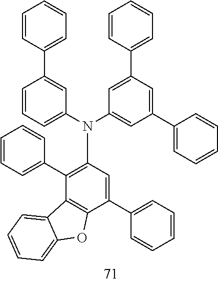 [SMILES: c1ccc(-c2cccc(N(c3cc(-c4ccccc4)cc(-c4ccccc4)c3)c3cc(-c4ccccc4)c4oc5ccccc5c4c3-c3ccccc3)c2)cc1] | 716.29 | 51 |
| 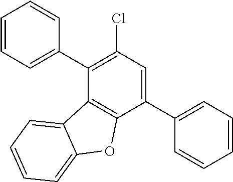 [SMILES: Clc1cc(-c2ccccc2)c2oc3ccccc3c2c1-c1ccccc1] Sub-b2 | 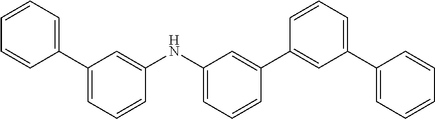 [SMILES: c1ccc(-c2cccc(Nc3cccc(-c4cccc(-c5ccccc5)c4)c3)c2)cc1] CAS: 2707395-17-1 | 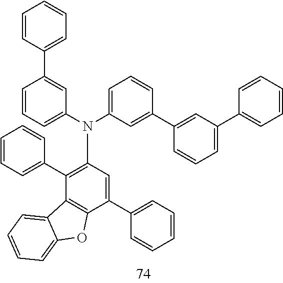 [SMILES: c1ccc(-c2cccc(-c3cccc(N(c4cccc(-c5ccccc5)c4)c4cc(-c5ccccc5)c5oc6ccccc6c5c4-c4ccccc4)c3)c2)cc1] | 716.29 | 54 |
| 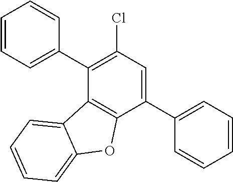 [SMILES: Clc1cc(-c2ccccc2)c2oc3ccccc3c2c1-c1ccccc1] Sub-b2 | 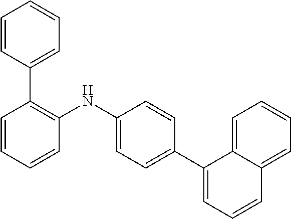 [SMILES: c1ccc(-c2ccccc2Nc2ccc(-c3cccc4ccccc34)cc2)cc1] CAS: 1848987-46-1 | 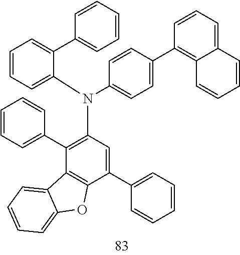 [SMILES: c1ccc(-c2ccccc2N(c2ccc(-c3cccc4ccccc34)cc2)c2cc(-c3ccccc3)c3oc4ccccc4c3c2-c2ccccc2)cc1] | 690.27 | 49 |
| 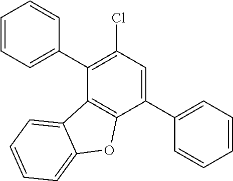 [SMILES: Clc1cc(-c2ccccc2)c2oc3ccccc3c2c1-c1ccccc1] Sub-b2 | 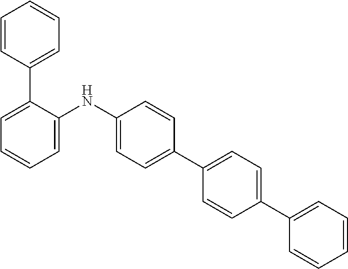 [SMILES: c1ccc(-c2ccc(-c3ccc(Nc4ccccc4-c4ccccc4)cc3)cc2)cc1] CAS: 1401351-59-4 | 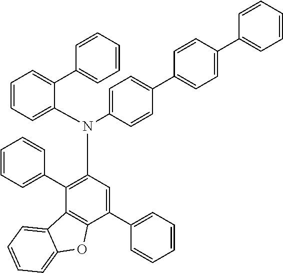 [SMILES: c1ccc(-c2ccc(-c3ccc(N(c4ccccc4-c4ccccc4)c4cc(-c5ccccc5)c5oc6ccccc6c5c4-c4ccccc4)cc3)cc2)cc1] 89 | 716.29 | 54 |
| 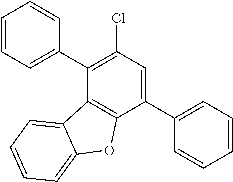 [SMILES: Clc1cc(-c2ccccc2)c2oc3ccccc3c2c1-c1ccccc1] Sub-b2 | 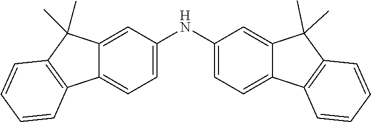 [SMILES: CC1(C)c2ccccc2-c2ccc(Nc3ccc4c(c3)C(C)(C)c3ccccc3-4)cc21] CAS: 500717-23-7 | 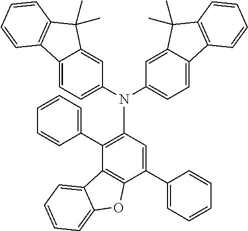 [SMILES: CC1(C)c2ccccc2-c2ccc(N(c3ccc4c(c3)C(C)(C)c3ccccc3-4)c3cc(-c4ccccc4)c4oc5ccccc5c4c3-c3ccccc3)cc21] 108 | 720.32 | 55 |
| 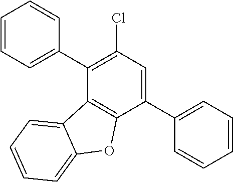 [SMILES: Clc1cc(-c2ccccc2)c2oc3ccccc3c2c1-c1ccccc1] Sub-b2 | 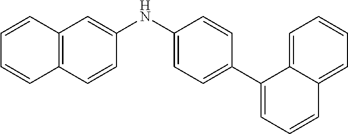 [SMILES: c1ccc2cc(Nc3ccc(-c4cccc5ccccc45)cc3)ccc2c1] CAS: 897921-58-3 | 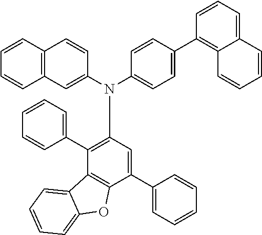 123 | 664.26 | 55 |
| 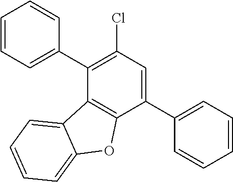 [SMILES: Clc1cc(-c2ccccc2)c2oc3ccccc3c2c1-c1ccccc1] Sub-b2 | 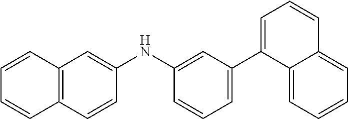 [SMILES: c1cc(Nc2ccc3ccccc3c2)cc(-c2cccc3ccccc23)c1] CAS: 2634130-32-6 | 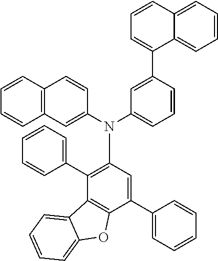 [SMILES: c1ccc(-c2cc(N(c3cccc(-c4cccc5ccccc45)c3)c3ccc4ccccc4c3)c(-c3ccccc3)c3c2oc2ccccc23)cc1] 125 | 664.26 | 48 |
| 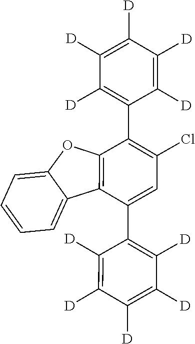 [SMILES: [2H]c1c([2H])c([2H])c(-c2c(Cl)cc(-c3c([2H])c([2H])c([2H])c([2H])c3[2H])c3c2oc2ccccc23)c([2H])c1[2H]] Sub-b8 | 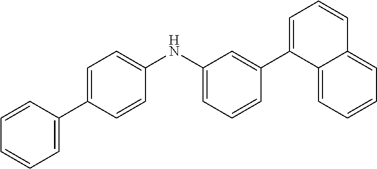 [SMILES: c1ccc(-c2ccc(Nc3cccc(-c4cccc5ccccc45)c3)cc2)cc1] CAS: 2634130-29-1 | 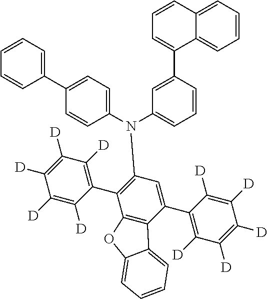 [SMILES: [2H]c1c([2H])c([2H])c(-c2c(N(c3ccc(-c4ccccc4)cc3)c3cccc(-c4cccc5ccccc45)c3)cc(-c3c([2H])c([2H])c([2H])c([2H])c3[2H])c3c2oc2ccccc23)c([2H])c1[2H]] 160 | 700.34 | 47 |
| 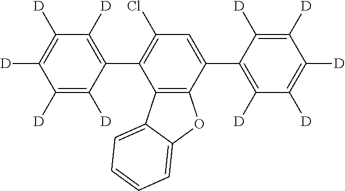 [SMILES: [2H]c1c([2H])c([2H])c(-c2cc(Cl)c(-c3c([2H])c([2H])c([2H])c([2H])c3[2H])c3c2oc2ccccc23)c([2H])c1[2H]] Sub-b9 | 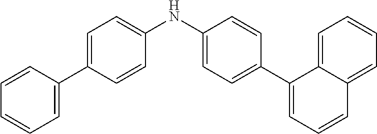 [SMILES: c1ccc(-c2ccc(Nc3ccc(-c4cccc5ccccc45)cc3)cc2)cc1] CAS: 897921-59-4 | 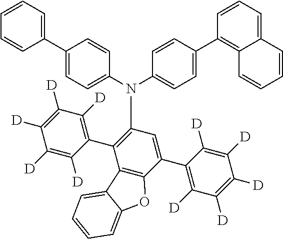 [SMILES: [2H]c1c([2H])c([2H])c(-c2cc(N(c3ccc(-c4ccccc4)cc3)c3ccc(-c4cccc5ccccc45)cc3)c(-c3c([2H])c([2H])c([2H])c([2H])c3[2H])c3c2oc2ccccc23)c([2H])c1[2H]] 38 | 700.34 | 50 |
| 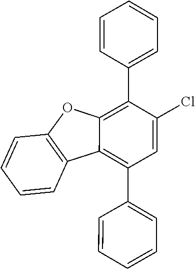 [SMILES: Clc1cc(-c2ccccc2)c2c(oc3ccccc32)c1-c1ccccc1] Sub-b1 | 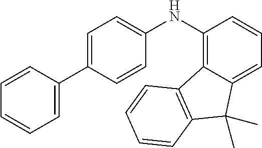 [SMILES: CC1(C)c2ccccc2-c2c(Nc3ccc(-c4ccccc4)cc3)cccc21] CAS: 1421789-16-3 | 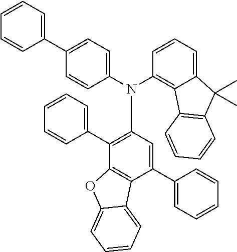 [SMILES: CC1(C)c2ccccc2-c2c(N(c3ccc(-c4ccccc4)cc3)c3cc(-c4ccccc4)c4c(oc5ccccc54)c3-c3ccccc3)cccc21] 155 | 680.29 | 53 |
| 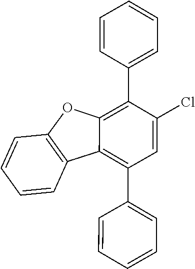 [SMILES: Clc1cc(-c2ccccc2)c2c(oc3ccccc32)c1-c1ccccc1] Sub-b1 | 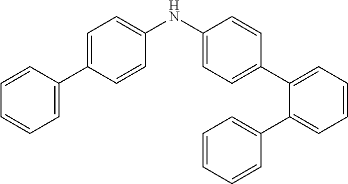 [SMILES: c1ccc(-c2ccc(Nc3ccc(-c4ccccc4-c4ccccc4)cc3)cc2)cc1] CAS: 1547491-61-1 | 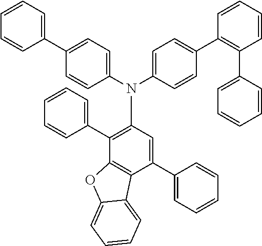 [SMILES: c1ccc(-c2ccc(N(c3ccc(-c4ccccc4-c4ccccc4)cc3)c3cc(-c4ccccc4)c4c(oc5ccccc54)c3-c3ccccc3)cc2)cc1] 163 | 716.29 | 45 |
| 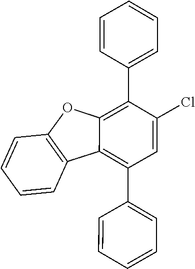 [SMILES: Clc1cc(-c2ccccc2)c2c(oc3ccccc32)c1-c1ccccc1] Sub-b1 | 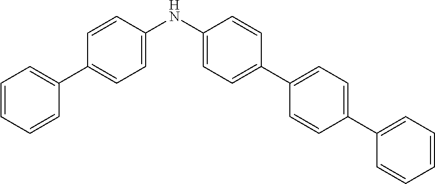 [SMILES: c1ccc(-c2ccc(Nc3ccc(-c4ccc(-c5ccccc5)cc4)cc3)cc2)cc1] CAS: 897921-63-0 | 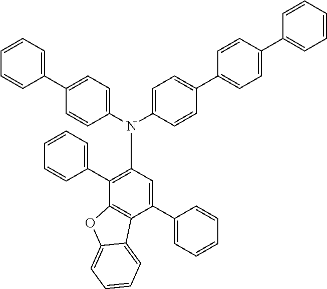 [SMILES: c1ccc(-c2ccc(-c3ccc(N(c4ccc(-c5ccccc5)cc4)c4cc(-c5ccccc5)c5c(oc6ccccc65)c4-c4ccccc4)cc3)cc2)cc1] 164 | 716.29 | 48 |
| 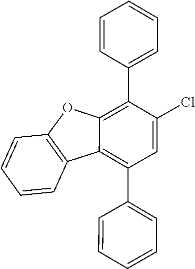 [SMILES: Clc1cc(-c2ccccc2)c2c(oc3ccccc32)c1-c1ccccc1] Sub-b1 | 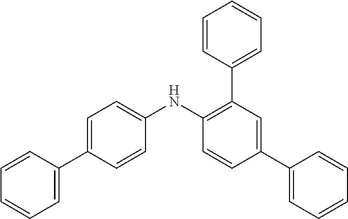 [SMILES: c1ccc(-c2ccc(Nc3ccc(-c4ccccc4)cc3-c3ccccc3)cc2)cc1] CAS: 1547491-85-9 | 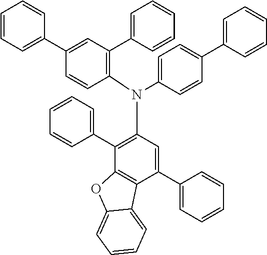 [SMILES: c1ccc(-c2ccc(N(c3ccc(-c4ccccc4)cc3-c3ccccc3)c3cc(-c4ccccc4)c4c(oc5ccccc54)c3-c3ccccc3)cc2)cc1] 167 | 716.29 | 43 |
| 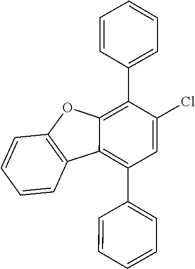 [SMILES: Clc1cc(-c2ccccc2)c2c(oc3ccccc32)c1-c1ccccc1] Sub-b1 | 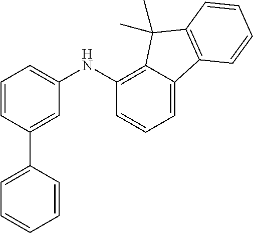 [SMILES: CC1(C)c2ccccc2-c2cccc(Nc3cccc(-c4ccccc4)c3)c21] CAS: 1644054-09-0 | 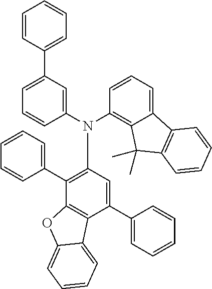 [SMILES: CC1(C)c2ccccc2-c2cccc(N(c3cccc(-c4ccccc4)c3)c3cc(-c4ccccc4)c4c(oc5ccccc54)c3-c3ccccc3)c21] 173 | 680.29 | 44 |
| 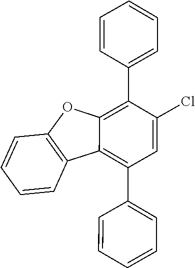 [SMILES: Clc1cc(-c2ccccc2)c2c(oc3ccccc32)c1-c1ccccc1] Sub-b1 | 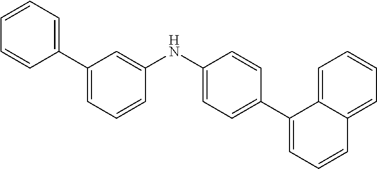 [SMILES: c1ccc(-c2cccc(Nc3ccc(-c4cccc5ccccc45)cc3)c2)cc1] CAS: 1446448-96-9 | 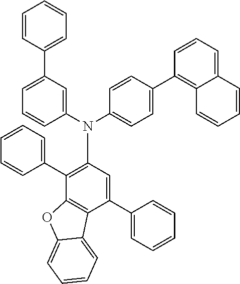 [SMILES: c1ccc(-c2cccc(N(c3ccc(-c4cccc5ccccc45)cc3)c3cc(-c4ccccc4)c4c(oc5ccccc54)c3-c3ccccc3)c2)cc1] 175 | 690.28 | 52 |
| 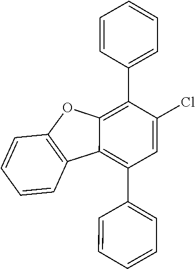 [SMILES: Clc1cc(-c2ccccc2)c2c(oc3ccccc32)c1-c1ccccc1] Sub-b1 | 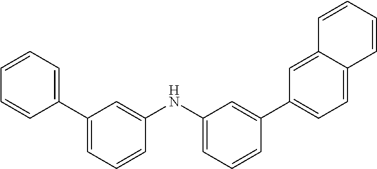 [SMILES: c1ccc(-c2cccc(Nc3cccc(-c4ccc5ccccc5c4)c3)c2)cc1] CAS: 1848987-47-2 | 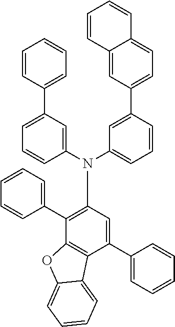 [SMILES: c1ccc(-c2cccc(N(c3cccc(-c4ccc5ccccc5c4)c3)c3cc(-c4ccccc4)c4c(oc5ccccc54)c3-c3ccccc3)c2)cc1] 178 | 690.28 | 50 |
| 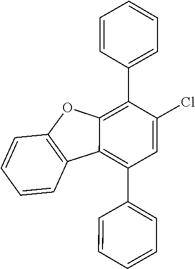 [SMILES: Clc1cc(-c2ccccc2)c2c(oc3ccccc32)c1-c1ccccc1] Sub-b1 | 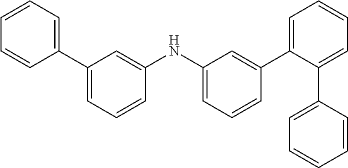 [SMILES: c1ccc(-c2cccc(Nc3cccc(-c4ccccc4-c4ccccc4)c3)c2)cc1] CAS: 2609809-30-3 | 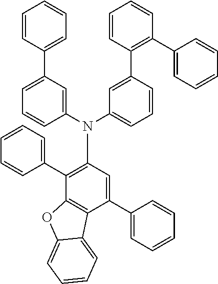 [SMILES: c1ccc(-c2cccc(N(c3cccc(-c4ccccc4-c4ccccc4)c3)c3cc(-c4ccccc4)c4c(oc5ccccc54)c3-c3ccccc3)c2)cc1] 179 | 716.29 | 46 |
| 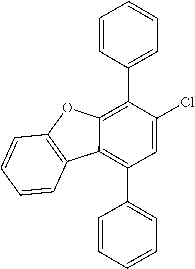 [SMILES: Clc1cc(-c2ccccc2)c2c(oc3ccccc32)c1-c1ccccc1] Sub-b1 | 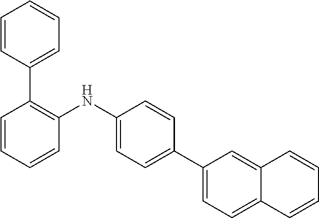 [SMILES: c1ccc(-c2ccccc2Nc2ccc(-c3ccc4ccccc4c3)cc2)cc1] CAS: 2135820-18-5 | 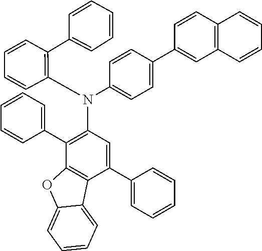 [SMILES: c1ccc(-c2ccccc2N(c2ccc(-c3ccc4ccccc4c3)cc2)c2cc(-c3ccccc3)c3c(oc4ccccc43)c2-c2ccccc2)cc1] 188 | 690.28 | 49 |
| 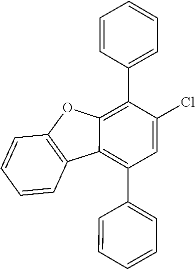 [SMILES: Clc1cc(-c2ccccc2)c2c(oc3ccccc32)c1-c1ccccc1] Sub-b1 | 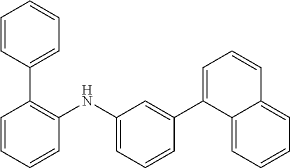 [SMILES: c1ccc(-c2ccccc2Nc2cccc(-c3cccc4ccccc34)c2)cc1] CAS: 2722640-39-1 | 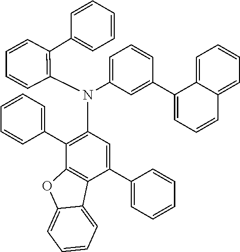 [SMILES: c1ccc(-c2ccccc2N(c2cccc(-c3cccc4ccccc34)c2)c2cc(-c3ccccc3)c3c(oc4ccccc43)c2-c2ccccc2)cc1] 189 | 690.28 | 52 |
| 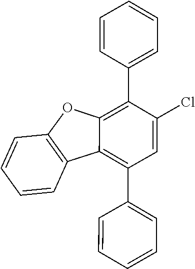 [SMILES: Clc1cc(-c2ccccc2)c2c(oc3ccccc32)c1-c1ccccc1] Sub-b1 | 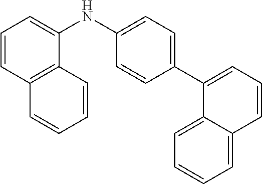 [SMILES: c1ccc2c(Nc3ccc(-c4cccc5ccccc45)cc3)cccc2c1] CAS: 936916-07-3 | 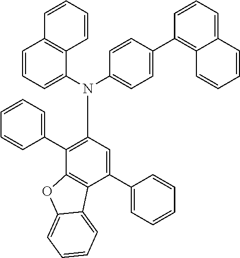 212 | 664.26 | 49 |
| 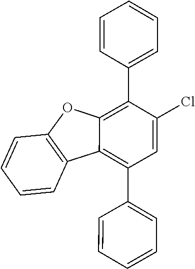 [SMILES: Clc1cc(-c2ccccc2)c2c(oc3ccccc32)c1-c1ccccc1] Sub-b1 | 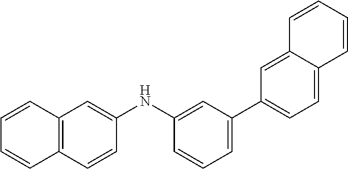 [SMILES: c1cc(Nc2ccc3ccccc3c2)cc(-c2ccc3ccccc3c2)c1] CAS: 1572044-47-3 | 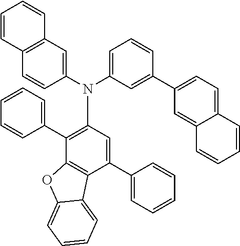 [SMILES: c1ccc(-c2c(N(c3cccc(-c4ccc5ccccc5c4)c3)c3ccc4ccccc4c3)cc(-c3ccccc3)c3c2oc2ccccc23)cc1] 215 | 664.26 | 54 |
| 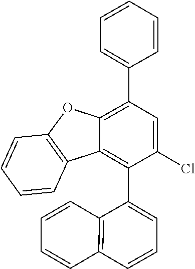 [SMILES: Clc1cc(-c2ccccc2)c2oc3ccccc3c2c1-c1cccc2ccccc12] Sub-b3 | 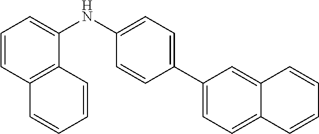 [SMILES: c1ccc2cc(-c3ccc(Nc4cccc5ccccc45)cc3)ccc2c1] CAS: 1400923-26-3 | 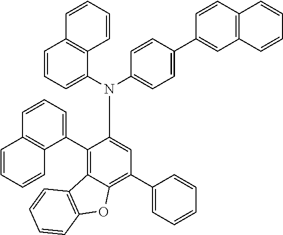 418 | 714.28 | 49 |
| 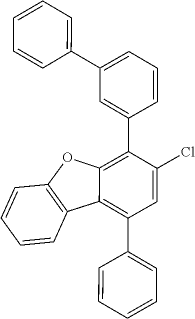 [SMILES: Clc1cc(-c2ccccc2)c2c(oc3ccccc32)c1-c1cccc(-c2ccccc2)c1] Sub-b5 | 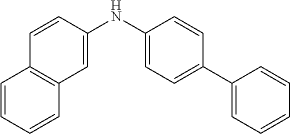 [SMILES: c1ccc(-c2ccc(Nc3ccc4ccccc4c3)cc2)cc1] CAS: 6336-92-1 | 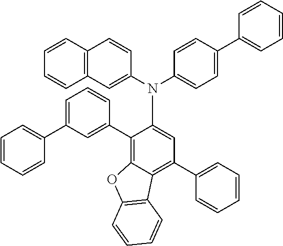 [SMILES: c1ccc(-c2ccc(N(c3ccc4ccccc4c3)c3cc(-c4ccccc4)c4c(oc5ccccc54)c3-c3cccc(-c4ccccc4)c3)cc2)cc1] 445 | 690.28 | 52 |

### 6. Synthesis of Compound 248

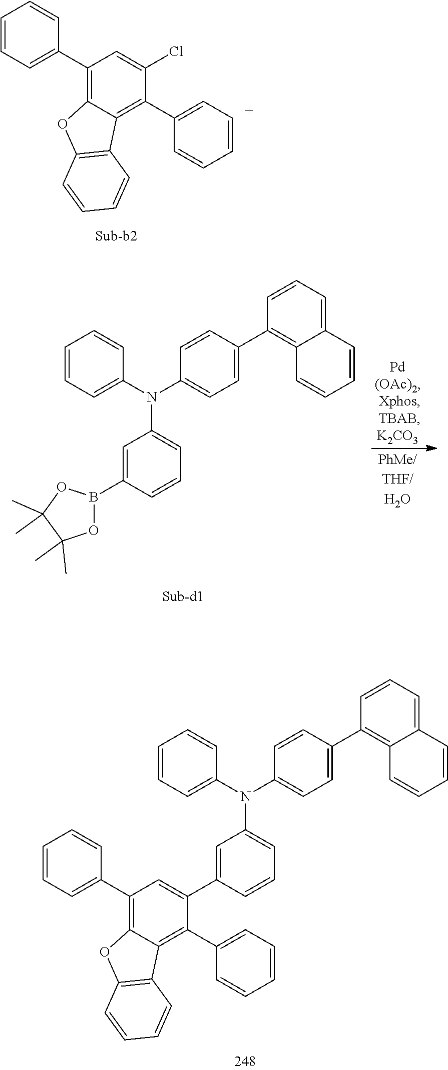 [SMILES: Clc1cc(-c2ccccc2)c2oc3ccccc3c2c1-c1ccccc1 ; CC1(C)OB(c2cccc(N(c3ccccc3)c3ccc(-c4cccc5ccccc45)cc3)c2)OC1(C)C ; c1ccc(-c2cc(-c3cccc(N(c4ccccc4)c4ccc(-c5cccc6ccccc56)cc4)c3)c(-c3ccccc3)c3c2oc2ccccc23)cc1]

Under a nitrogen atmosphere, to a 250 mL three-necked flask was sequentially added Sub-b2 (8.87 g, 25 mmol), Sub-d1 (13.68 g, 27.5 mmol), palladium acetate (0.083 g, 0.5 mmol), 2-dicyclohexylphosphino-2′,4′,6′-triisopropylbiphenyl (0.48 g, 1 mmol), tetrabutylammonium bromide (0.81 g, 2.5 mmol), potassium carbonate (6.91 g, 50 mmol), toluene (100 mL), tetrahydrofuran (25 mL), and deionized water (25 mL). The resulting mixture was heated to reflux and react overnight with stirring. After the reaction solution was cooled to room temperature, it was extracted with dichloromethane (100 mL×3 times), and the organic phases were combined and dried on anhydrous magnesium sulfate, followed by filtration and then removal of the solvent via distillation under reduced pressure, to obtain a crude product. The crude product was purified by silica gel column chromatography using dichloromethane/n-hexane as the mobile phase, to obtain a white solid (13.45 g, yield 78%), Mass Spectra (m/z)=690.28 [M+H]⁺.

The compounds listed in Table 6 below were synthesized using the same method as Compound 248, except that Reactant K was used instead of Sub-b2 and Reactant L was used instead of RM-3. The main raw materials used, the synthesized intermediates, and their Mass Spectra yields are shown in Table 6.

TABLE 6

| | | | Mass | |
|---|---|---|---|---|
| | | | Spectra | |
| | | | (m/z)/ | Yield |
| Reactant K | Reactant L | Compound | [M + H]+ | (%) |
| 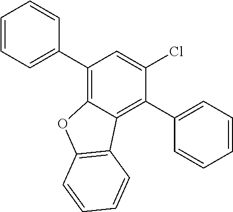 [SMILES: Clc1cc(-c2ccccc2)c2oc3ccccc3c2c1-c1ccccc1] Sub-b2 | 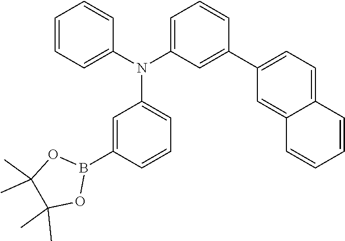 [SMILES: CC1(C)OB(c2cccc(N(c3ccccc3)c3cccc(-c4ccc5ccccc5c4)c3)c2)OC1(C)C] Sub-d2 | 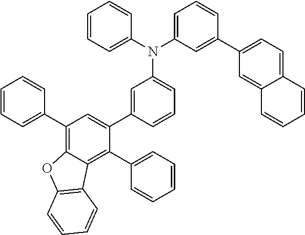 [SMILES: c1ccc(-c2cc(-c3cccc(N(c4ccccc4)c4cccc(-c5ccc6ccccc6c5)c4)c3)c(-c3ccccc3)c3c2oc2ccccc23)cc1] 251 | 690.28 | 71 |
| 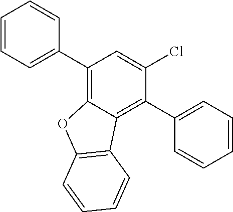 [SMILES: Clc1cc(-c2ccccc2)c2oc3ccccc3c2c1-c1ccccc1] Sub-b2 | 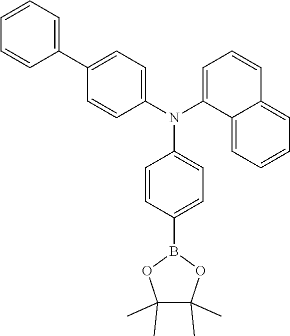 [SMILES: CC1(C)OB(c2ccc(N(c3ccc(-c4ccccc4)cc3)c3cccc4ccccc34)cc2)OC1(C)C] Sub-d3 | 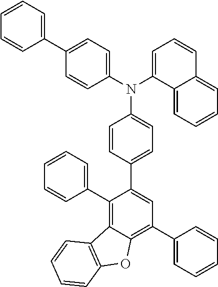 [SMILES: c1ccc(-c2ccc(N(c3ccc(-c4cc(-c5ccccc5)c5oc6ccccc6c5c4-c4ccccc4)cc3)c3cccc4ccccc34)cc2)cc1] 259 | 690.28 | 72 |
| 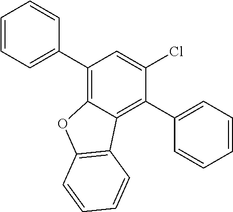 [SMILES: Clc1cc(-c2ccccc2)c2oc3ccccc3c2c1-c1ccccc1] Sub-b2 | 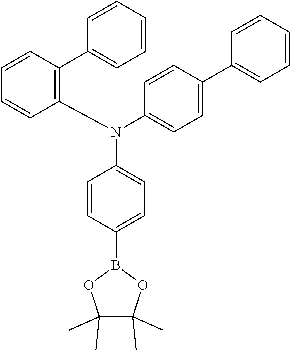 [SMILES: CC1(C)OB(c2ccc(N(c3ccc(-c4ccccc4)cc3)c3ccccc3-c3ccccc3)cc2)OC1(C)C] Sub-d4 | 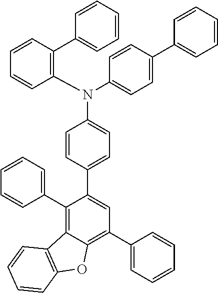 [SMILES: c1ccc(-c2ccc(N(c3ccc(-c4cc(-c5ccccc5)c5oc6ccccc6c5c4-c4ccccc4)cc3)c3ccccc3-c3ccccc3)cc2)cc1] 261 | 716.30 | 67 |
| 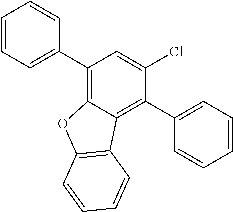 [SMILES: Clc1cc(-c2ccccc2)c2oc3ccccc3c2c1-c1ccccc1] Sub-b2 | 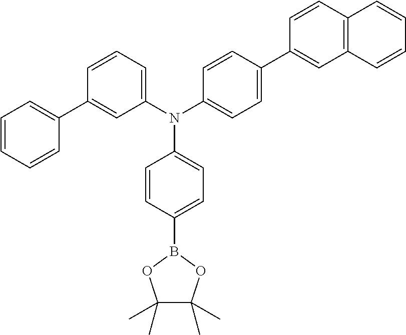 [SMILES: CC1(C)OB(c2ccc(N(c3ccc(-c4ccc5ccccc5c4)cc3)c3cccc(-c4ccccc4)c3)cc2)OC1(C)C] Sub-d6 | 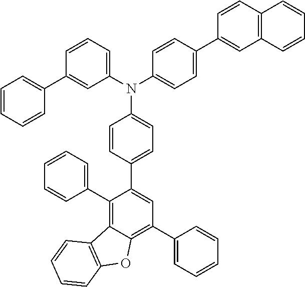 [SMILES: c1ccc(-c2cccc(N(c3ccc(-c4ccc5ccccc5c4)cc3)c3ccc(-c4cc(-c5ccccc5)c5oc6ccccc6c5c4-c4ccccc4)cc3)c2)cc1] 269 | 766.31 | 72 |
| 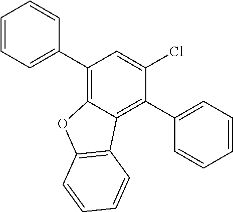 [SMILES: Clc1cc(-c2ccccc2)c2oc3ccccc3c2c1-c1ccccc1] Sub-b2 | 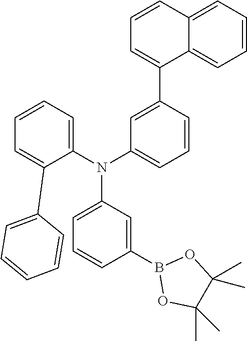 [SMILES: CC1(C)OB(c2cccc(N(c3cccc(-c4cccc5ccccc45)c3)c3ccccc3-c3ccccc3)c2)OC1(C)C] Sub-d7 | 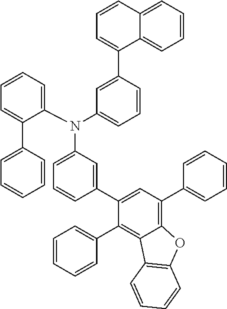 [SMILES: c1ccc(-c2ccccc2N(c2cccc(-c3cc(-c4ccccc4)c4oc5ccccc5c4c3-c3ccccc3)c2)c2cccc(-c3cccc4ccccc34)c2)cc1] 270 | 766.31 | 63 |
| 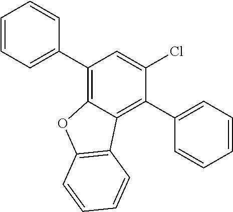 [SMILES: Clc1cc(-c2ccccc2)c2oc3ccccc3c2c1-c1ccccc1] Sub-b2 | 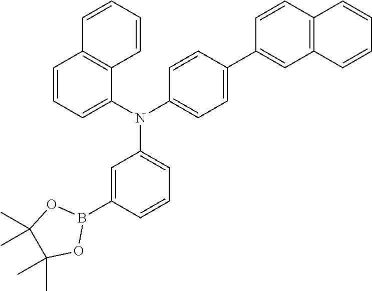 [SMILES: CC1(C)OB(c2cccc(N(c3ccc(-c4ccc5ccccc5c4)cc3)c3cccc4ccccc34)c2)OC1(C)C] Sub-d9 | 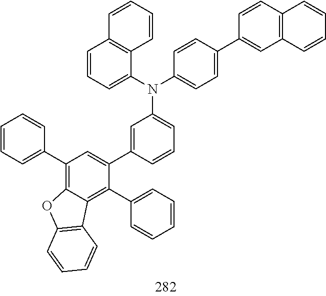 [SMILES: c1ccc(-c2cc(-c3cccc(N(c4ccc(-c5ccc6ccccc6c5)cc4)c4cccc5ccccc45)c3)c(-c3ccccc3)c3c2oc2ccccc23)cc1] | 740.29 | 73 |
| 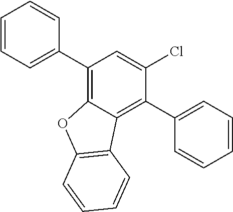 [SMILES: Clc1cc(-c2ccccc2)c2oc3ccccc3c2c1-c1ccccc1] Sub-b2 | 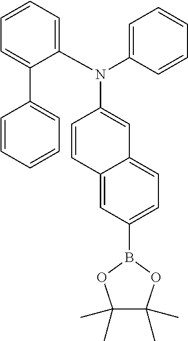 [SMILES: CC1(C)OB(c2ccc3cc(N(c4ccccc4)c4ccccc4-c4ccccc4)ccc3c2)OC1(C)C] Sub-d13 | 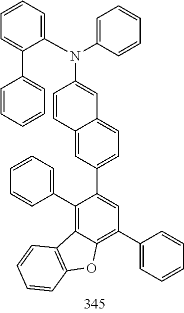 [SMILES: c1ccc(-c2ccccc2N(c2ccccc2)c2ccc3cc(-c4cc(-c5ccccc5)c5oc6ccccc6c5c4-c4ccccc4)ccc3c2)cc1] | 690.28 | 71 |
| 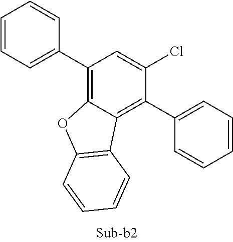 [SMILES: Clc1cc(-c2ccccc2)c2oc3ccccc3c2c1-c1ccccc1] Sub-b2 | 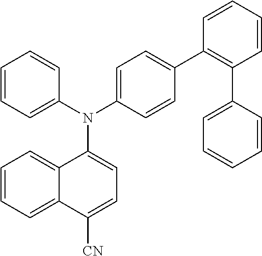 [SMILES: N#Cc1ccc(N(c2ccccc2)c2ccc(-c3ccccc3-c3ccccc3)cc2)c2ccccc12] Sub-d14 | 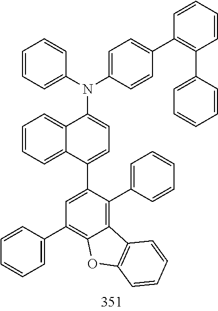 [SMILES: c1ccc(-c2ccccc2-c2ccc(N(c3ccccc3)c3ccc(-c4cc(-c5ccccc5)c5oc6ccccc6c5c4-c4ccccc4)c4ccccc34)cc2)cc1] | 766.31 | 68 |
| 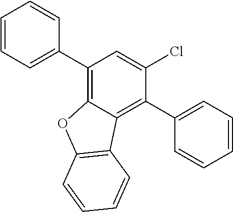 [SMILES: Clc1cc(-c2ccccc2)c2oc3ccccc3c2c1-c1ccccc1] Sub-b2 | 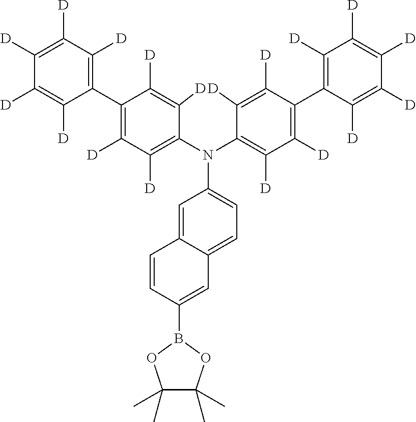 [SMILES: [2H]c1c([2H])c([2H])c(-c2c([2H])c([2H])c(N(c3ccc4cc(B5OC(C)(C)C(C)(C)O5)ccc4c3)c3c([2H])c([2H])c(-c4c([2H])c([2H])c([2H])c([2H])c4[2H])c([2H])c3[2H])c([2H])c2[2H])c([2H])c1[2H]] Sub-d16 | 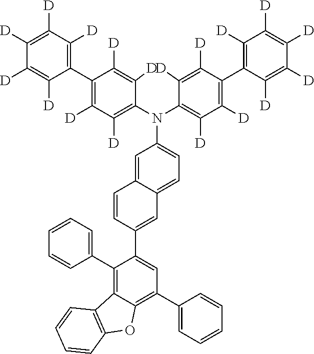 [SMILES: [2H]c1c([2H])c([2H])c(-c2c([2H])c([2H])c(N(c3ccc4cc(-c5cc(-c6ccccc6)c6oc7ccccc7c6c5-c5ccccc5)ccc4c3)c3c([2H])c([2H])c(-c4c([2H])c([2H])c([2H])c([2H])c4[2H])c([2H])c3[2H])c([2H])c2[2H])c([2H])c1[2H]] 361 | 784.42 | 66 |
| 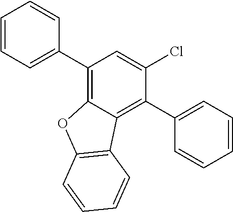 [SMILES: Clc1cc(-c2ccccc2)c2oc3ccccc3c2c1-c1ccccc1] Sub-b2 | 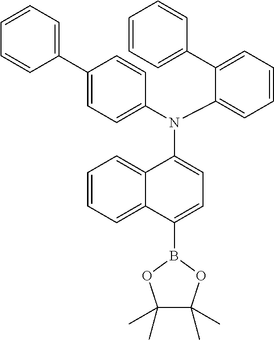 [SMILES: CC1(C)OB(c2ccc(N(c3ccc(-c4ccccc4)cc3)c3ccccc3-c3ccccc3)c3ccccc23)OC1(C)C] Sub-d17 | 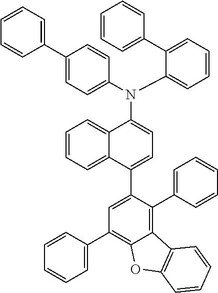 [SMILES: c1ccc(-c2ccc(N(c3ccccc3-c3ccccc3)c3ccc(-c4cc(-c5ccccc5)c5oc6ccccc6c5c4-c4ccccc4)c4ccccc34)cc2)cc1] 363 | 766.31 | 72 |
| 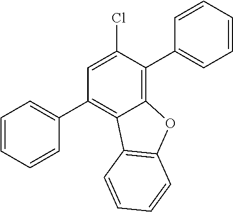 [SMILES: Clc1cc(-c2ccccc2)c2c(oc3ccccc32)c1-c1ccccc1] Sub-b1 | 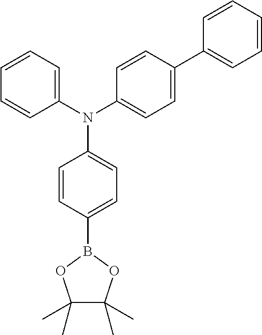 [SMILES: CC1(C)OB(c2ccc(N(c3ccccc3)c3ccc(-c4ccccc4)cc3)cc2)OC1(C)C] Sub-d10 | 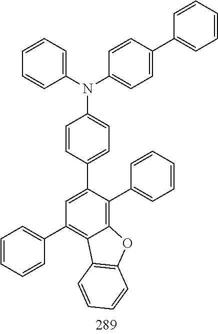 [SMILES: c1ccc(-c2ccc(N(c3ccccc3)c3ccc(-c4cc(-c5ccccc5)c5c(oc6ccccc65)c4-c4ccccc4)cc3)cc2)cc1] | 640.26 | 74 |
| 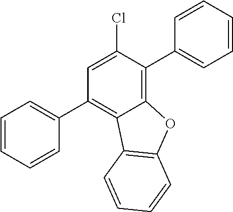 [SMILES: Clc1cc(-c2ccccc2)c2c(oc3ccccc32)c1-c1ccccc1] Sub-b1 | 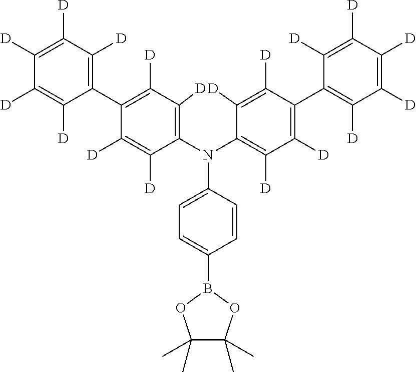 [SMILES: [2H]c1c([2H])c([2H])c(-c2c([2H])c([2H])c(N(c3ccc(B4OC(C)(C)C(C)(C)O4)cc3)c3c([2H])c([2H])c(-c4c([2H])c([2H])c([2H])c([2H])c4[2H])c([2H])c3[2H])c([2H])c2[2H])c([2H])c1[2H]] Sub-d12 | 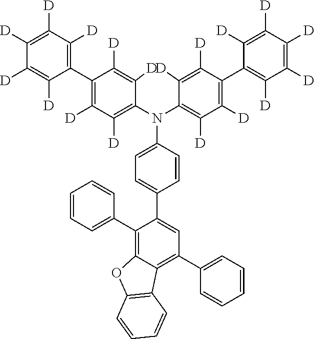 [SMILES: [2H]c1c([2H])c([2H])c(-c2c([2H])c([2H])c(N(c3ccc(-c4cc(-c5ccccc5)c5c(oc6ccccc65)c4-c4ccccc4)cc3)c3c([2H])c([2H])c(-c4c([2H])c([2H])c([2H])c([2H])c4[2H])c([2H])c3[2H])c([2H])c2[2H])c([2H])c1[2H]] 328 | 734.40 | 73 |
| 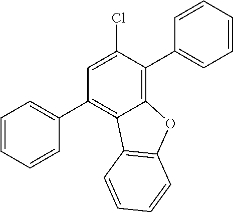 [SMILES: Clc1cc(-c2ccccc2)c2c(oc3ccccc32)c1-c1ccccc1] Sub-b1 | 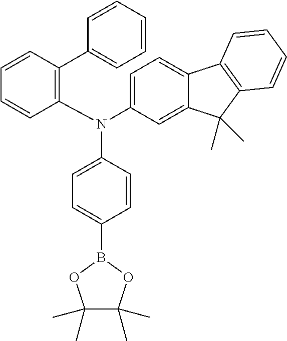 [SMILES: CC1(C)c2ccccc2-c2ccc(N(c3ccc(B4OC(C)(C)C(C)(C)O4)cc3)c3ccccc3-c3ccccc3)cc21] Sub-d5 | 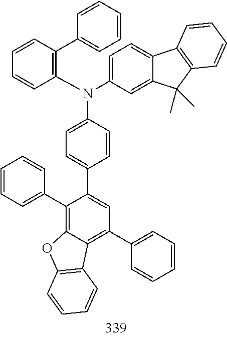 [SMILES: CC1(C)c2ccccc2-c2ccc(N(c3ccc(-c4cc(-c5ccccc5)c5c(oc6ccccc65)c4-c4ccccc4)cc3)c3ccccc3-c3ccccc3)cc21] | 756.32 | 70 |
| 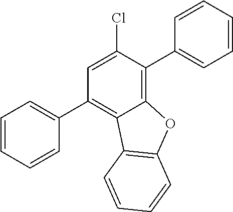 [SMILES: Clc1cc(-c2ccccc2)c2c(oc3ccccc32)c1-c1ccccc1] Sub-b1 | 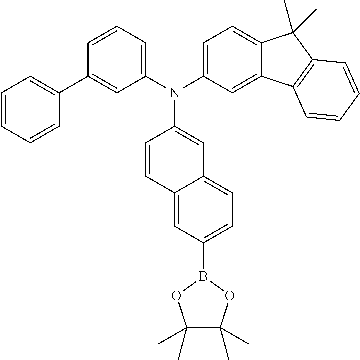 [SMILES: CC1(C)c2ccccc2-c2cc(N(c3cccc(-c4ccccc4)c3)c3ccc4cc(B5OC(C)(C)C(C)(C)O5)ccc4c3)ccc21] Sub-d18 | 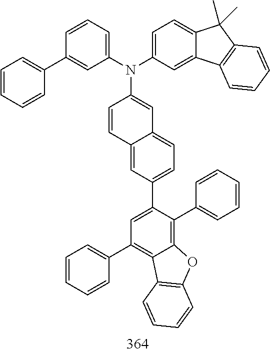 [SMILES: CC1(C)c2ccccc2-c2cc(N(c3cccc(-c4ccccc4)c3)c3ccc4cc(-c5cc(-c6ccccc6)c6c(oc7ccccc76)c5-c5ccccc5)ccc4c3)ccc21] | 806.34 | 75 |
| 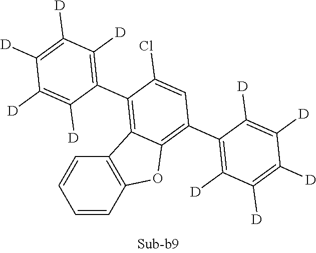 [SMILES: [2H]c1c([2H])c([2H])c(-c2cc(Cl)c(-c3c([2H])c([2H])c([2H])c([2H])c3[2H])c3c2oc2ccccc23)c([2H])c1[2H]] | 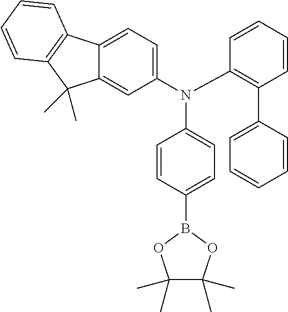 [SMILES: CC1(C)c2ccccc2-c2ccc(N(c3ccc(B4OC(C)(C)C(C)(C)O4)cc3)c3ccccc3-c3ccccc3)cc21] Sub-d5 | 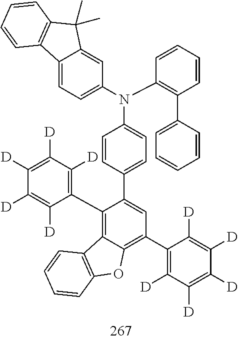 [SMILES: [2H]c1c([2H])c([2H])c(-c2cc(-c3ccc(N(c4ccc5c(c4)C(C)(C)c4ccccc4-5)c4ccccc4-c4ccccc4)cc3)c(-c3c([2H])c([2H])c([2H])c([2H])c3[2H])c3c2oc2ccccc23)c([2H])c1[2H]] | 766.38 | 66 |
| 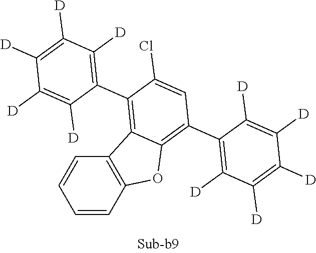 [SMILES: [2H]c1c([2H])c([2H])c(-c2cc(Cl)c(-c3c([2H])c([2H])c([2H])c([2H])c3[2H])c3c2oc2ccccc23)c([2H])c1[2H]] | 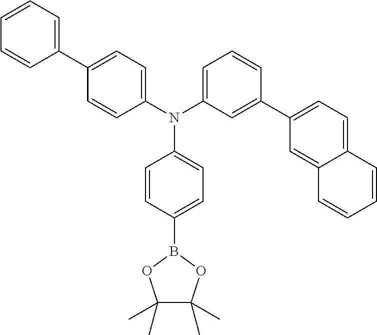 [SMILES: CC1(C)OB(c2ccc(N(c3ccc(-c4ccccc4)cc3)c3cccc(-c4ccc5ccccc5c4)c3)cc2)OC1(C)C] Sub-d8 | 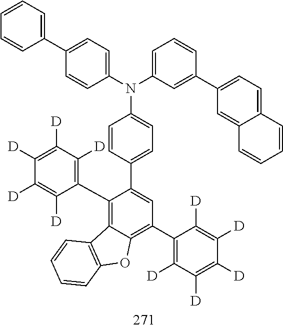 [SMILES: [2H]c1c([2H])c([2H])c(-c2cc(-c3ccc(N(c4ccc(-c5ccccc5)cc4)c4cccc(-c5ccc6ccccc6c5)c4)cc3)c(-c3c([2H])c([2H])c([2H])c([2H])c3[2H])c3c2oc2ccccc23)c([2H])c1[2H]] | 776.37 | 67 |
| 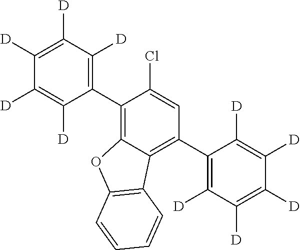 [SMILES: [2H]c1c([2H])c([2H])c(-c2c(Cl)cc(-c3c([2H])c([2H])c([2H])c([2H])c3[2H])c3c2oc2ccccc23)c([2H])c1[2H]] Sub-b8 | 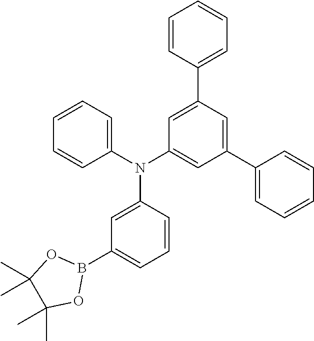 [SMILES: CC1(C)OB(c2cccc(N(c3ccccc3)c3cc(-c4ccccc4)cc(-c4ccccc4)c3)c2)OC1(C)C] Sub-d11 | 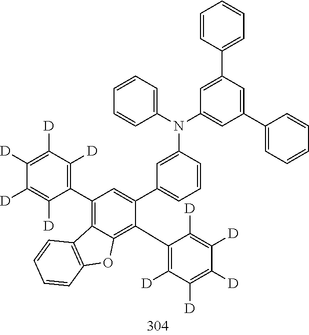 [SMILES: [2H]c1c([2H])c([2H])c(-c2c(-c3cccc(N(c4ccccc4)c4cc(-c5ccccc5)cc(-c5ccccc5)c4)c3)cc(-c3c([2H])c([2H])c([2H])c([2H])c3[2H])c3c2oc2ccccc23)c([2H])c1[2H]] | 726.35 | 65 |
| 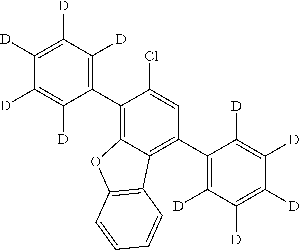 [SMILES: [2H]c1c([2H])c([2H])c(-c2c(Cl)cc(-c3c([2H])c([2H])c([2H])c([2H])c3[2H])c3c2oc2ccccc23)c([2H])c1[2H]] Sub-b8 | 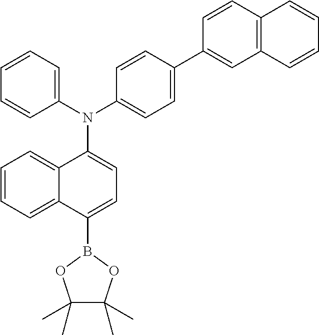 [SMILES: CC1(C)OB(c2ccc(N(c3ccccc3)c3ccc(-c4ccc5ccccc5c4)cc3)c3ccccc23)OC1(C)C] Sub-d15 | 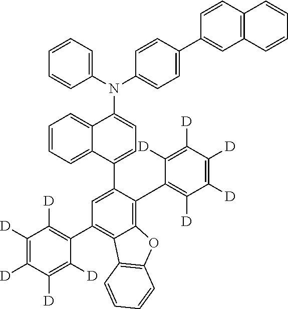 [SMILES: [2H]c1c([2H])c([2H])c(-c2c(-c3ccc(N(c4ccccc4)c4ccc(-c5ccc6ccccc6c5)cc4)c4ccccc34)cc(-c3c([2H])c([2H])c([2H])c([2H])c3[2H])c3c2oc2ccccc23)c([2H])c1[2H]] 358 | 750.35 | 70 |
| 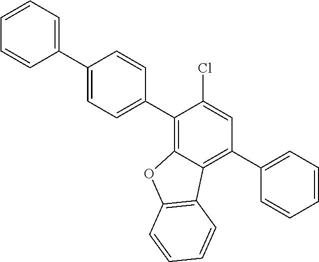 [SMILES: Clc1cc(-c2ccccc2)c2c(oc3ccccc32)c1-c1ccc(-c2ccccc2)cc1] Sub-b7 | 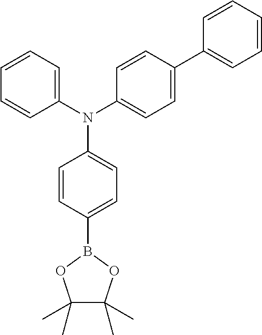 [SMILES: CC1(C)OB(c2ccc(N(c3ccccc3)c3ccc(-c4ccccc4)cc3)cc2)OC1(C)C] Sub-d10 | 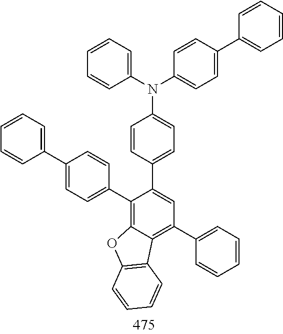 [SMILES: c1ccc(-c2ccc(-c3c(-c4ccc(N(c5ccccc5)c5ccc(-c6ccccc6)cc5)cc4)cc(-c4ccccc4)c4c3oc3ccccc34)cc2)cc1] | 716.29 | 71 |
| 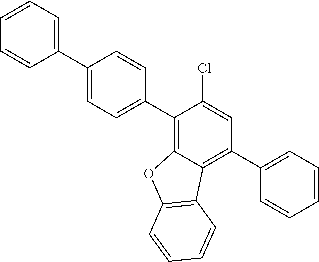 [SMILES: Clc1cc(-c2ccccc2)c2c(oc3ccccc32)c1-c1ccc(-c2ccccc2)cc1] Sub-b7 | 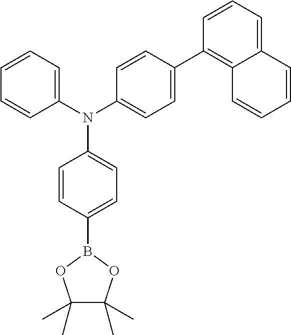 [SMILES: CC1(C)OB(c2ccc(N(c3ccccc3)c3ccc(-c4cccc5ccccc45)cc3)cc2)OC1(C)C] Sub-d20 | 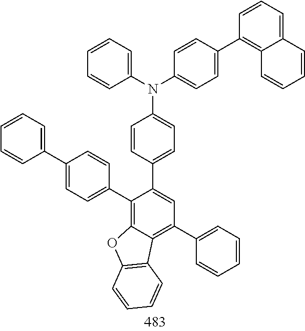 [SMILES: c1ccc(-c2ccc(-c3c(-c4ccc(N(c5ccccc5)c5ccc(-c6cccc7ccccc67)cc5)cc4)cc(-c4ccccc4)c4c3oc3ccccc34)cc2)cc1] | 766.30 | 65 |
| 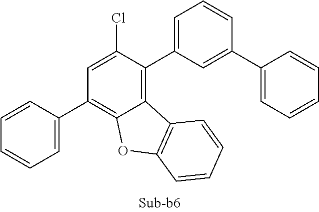 [SMILES: Clc1cc(-c2ccccc2)c2oc3ccccc3c2c1-c1cccc(-c2ccccc2)c1] | 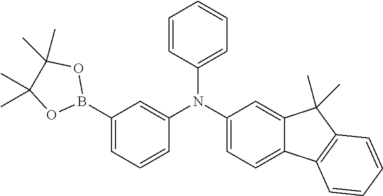 [SMILES: CC1(C)c2ccccc2-c2ccc(N(c3ccccc3)c3cccc(B4OC(C)(C)C(C)(C)O4)c3)cc21] Sub-d19 | 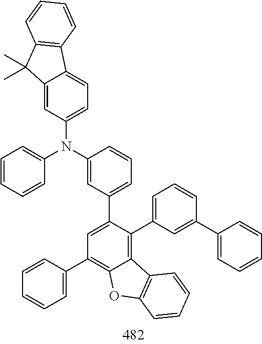 [SMILES: CC1(C)c2ccccc2-c2ccc(N(c3ccccc3)c3cccc(-c4cc(-c5ccccc5)c5oc6ccccc6c5c4-c4cccc(-c5ccccc5)c4)c3)cc21] | 756.32 | 67 |
| 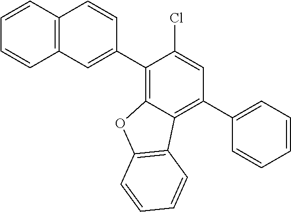 [SMILES: Clc1cc(-c2ccccc2)c2c(oc3ccccc32)c1-c1ccc2ccccc2c1] Sub-b4 | 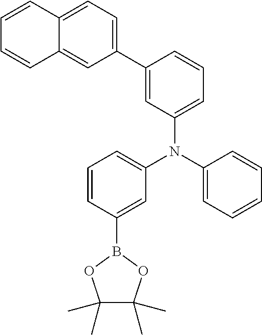 [SMILES: CC1(C)OB(c2cccc(N(c3ccccc3)c3cccc(-c4ccc5ccccc5c4)c3)c2)OC1(C)C] Sub-d2 | 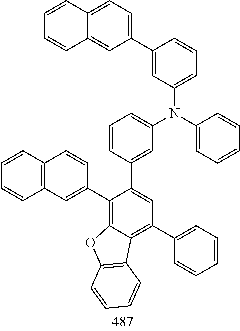 [SMILES: c1ccc(-c2cc(-c3cccc(N(c4ccccc4)c4cccc(-c5ccc6ccccc6c5)c4)c3)c(-c3ccc4ccccc4c3)c3oc4ccccc4c23)cc1] | 740.29 | 72 |

NMR data of Compound 251: ¹H-NMR (400 MHZ, Methylene-Chloride-D2) δ ppm 7.92 (d, 1H), 7.87-7.75 (m, 6H), 7.73-7.62 (m, 6H), 7.59-7.40 (m, 6H), 7.34 (t, 1H), 7.30-7.22 (m, 6H), 7.17 (d, 1H), 7.10 (t, 1H), 7.03 (s, 1H), 6.96 (s, 1H), 6.65 (t, 1H), 6.62-6.58 (m, 4H).

### Example 1: Red Organic Electroluminescent Device

The anode was prepared through the following process: An ITO/Ag/ITO substrate with thicknesses of 100 Å, 1000 Å, and 100 Å in order was cut into a size of 40 mm (length)×40 mm (width)×0.7 mm (height), and prepared into a test substrate having anode and insulation layer patterns with photolithography process. Ultraviolet ozone and O₂:N₂ plasma were used to perform surface treatment to increase the work function of the anode, and the surface of ITO substrates was cleaned using organic solvents to remove impurities and oil stains.

PD and HT-36 were co-vapor deposited on the test substrate (anode) at a vapor deposition rate ratio of 2%:98% to form a hole injection layer with a thickness of 100 Å.

The compound HT-36 was vacuum vapor deposited on the hole injection layer to form a first hole transport layer with a thickness of 1065 Å.

The compound HT-31 was vacuum vapor deposited on the first hole transport layer to form a second hole transport layer with a thickness of 890 Å.

The compound 11: RH-N: RD were co-vapor deposited on the second hole transport layer at a vapor deposition rate ratio of 49%:49%:2% to form an organic light-emitting layer with a thickness of 400 Å.

Compound ET-21 and LiQ were co-vapor deposited on the organic light-emitting layer at a vapor deposition rate ratio of 1:1 to form an electron transport layer (ETL) with a thickness of 350 Å. Yb was vapor deposited on the electron transport layer to form an electron injection layer (EIL) with a thickness of 10 Å. Then, magnesium (Mg) and silver (Ag) were mixed at a vapor deposition rate of 1:9, and vacuum vapor deposited on the electron injection layer to form a cathode with a thickness of 130 Å.

In addition, Compound CP-1 was vacuum vapor deposited on the above cathode to form an organic cappling layer with a thickness of 800 Å, thus completing the fabrication of the red organic electroluminescent device.

### Examples 2 to 30

Organic electroluminescent devices were fabricated by the same method as used in Example 1, except that Compound X listed in Table 5 below was used instead of the Compound 11 in Example 1 when forming the light-emitting layer.

### Comparative Examples 1 to 4

Organic electroluminescent devices were fabricated by the same method as used in Example 1, except that Compound A, Compound B, Compound C, and Compound D were used respectively instead of the Compound 11 in Example 1 when forming the light-emitting layer.

Among them, the compounds used to fabricate the devices of each Example and Comparative Example are listed in Table 7 below.

TABLE 7 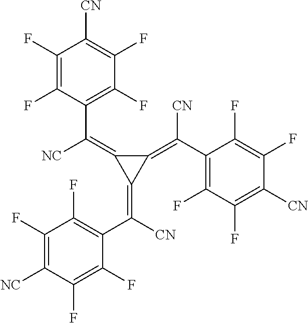 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]
PD 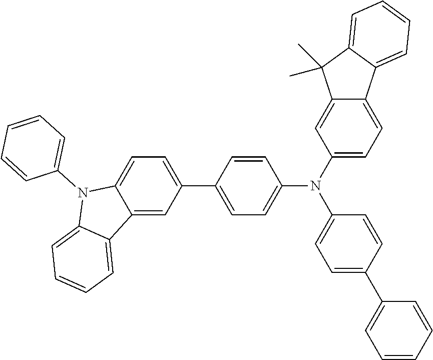 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]
HT-36 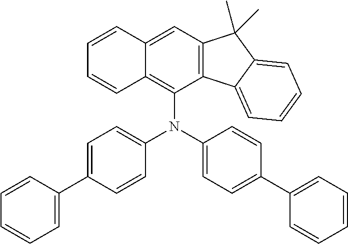 [SMILES: CC1(C)c2ccccc2-c2c1cc1ccccc1c2N(c1ccc(-c2ccccc2)cc1)c1ccc(-c2ccccc2)cc1]
HT-31 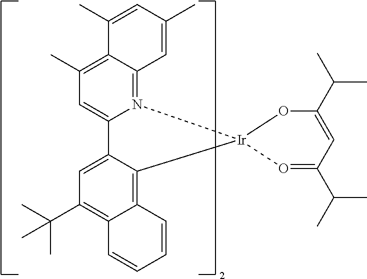
RD 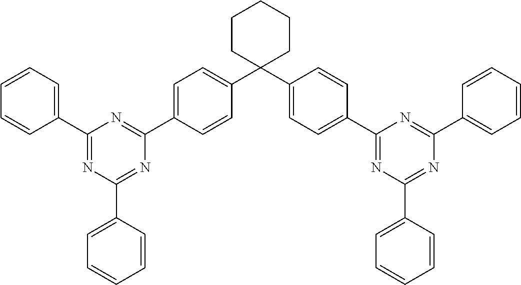 [SMILES: c1ccc(-c2nc(-c3ccccc3)nc(-c3ccc(C4(c5ccc(-c6nc(-c7ccccc7)nc(-c7ccccc7)n6)cc5)CCCCC4)cc3)n2)cc1]
ET-21 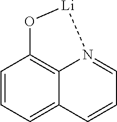
LiQ 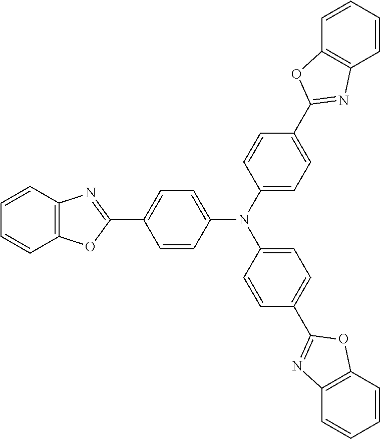 [SMILES: c1ccc2oc(-c3ccc(N(c4ccc(-c5nc6ccccc6o5)cc4)c4ccc(-c5nc6ccccc6o5)cc4)cc3)nc2c1]
CP-1 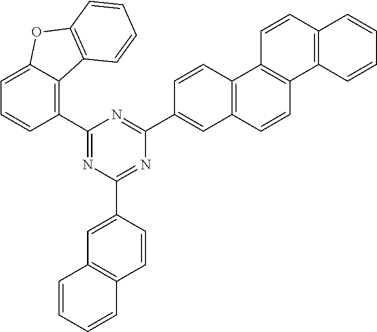 [SMILES: c1ccc2cc(-c3nc(-c4ccc5c(ccc6c7ccccc7ccc56)c4)nc(-c4cccc5oc6ccccc6c45)n3)ccc2c1]
RH-N 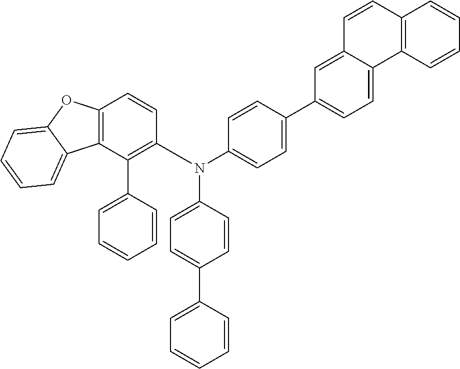 [SMILES: c1ccc(-c2ccc(N(c3ccc(-c4ccc5c(ccc6ccccc65)c4)cc3)c3ccc4oc5ccccc5c4c3-c3ccccc3)cc2)cc1]
Compound A 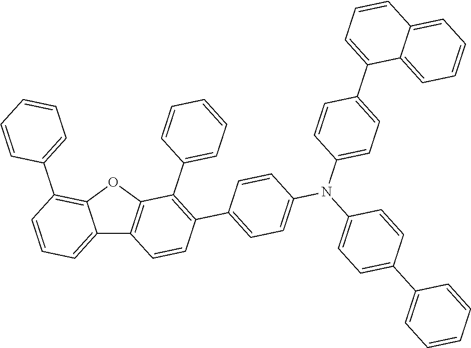 [SMILES: c1ccc(-c2ccc(N(c3ccc(-c4ccc5c(oc6c(-c7ccccc7)cccc65)c4-c4ccccc4)cc3)c3ccc(-c4cccc5ccccc45)cc3)cc2)cc1]
Compound B 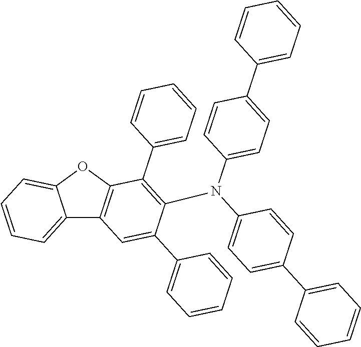 [SMILES: c1ccc(-c2ccc(N(c3ccc(-c4ccccc4)cc3)c3c(-c4ccccc4)cc4c(oc5ccccc54)c3-c3ccccc3)cc2)cc1]
Compound C 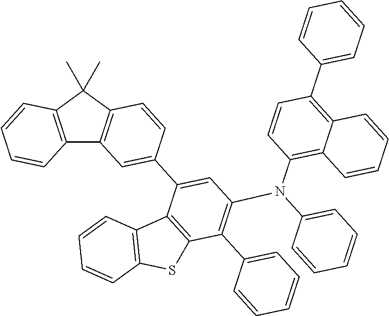 [SMILES: CC1(C)c2ccccc2-c2cc(-c3cc(N(c4ccccc4)c4ccc(-c5ccccc5)c5ccccc45)c(-c4ccccc4)c4sc5ccccc5c34)ccc21]
Compound D

Performance tests were conducted on the red organic electroluminescent devices fabricated in Examples 1 to 30 and Comparative Examples 1 to 4. IVL performance (driving voltage, current efficiency, color coordinates) was tested at 10 mA/cm², and T95 device lifetime was tested at 20 mA/cm². The results are shown in Table 8.

TABLE 8

| | Light-emitting | | | | |
|---|---|---|---|---|---|
| | layer | Driving | Current | Color | T95 |
| Example | Compound X: | Voltage | Efficiency | Coordinates | Lifetime |
| No. | RH-N:RD | (V) | (Cd/A) | CIEx, CIEy | (hrs) |
| Example 1 | Compound 11 | 3.49 | 56.31 | 0.680, 0.320 | 398 |
| Example 2 | Compound 12 | 3.57 | 55.73 | 0.680, 0.320 | 402 |
| Example 3 | Compound 38 | 3.38 | 56.17 | 0.680, 0.320 | 399 |
| Example 4 | Compound 43 | 3.50 | 56.25 | 0.680, 0.320 | 395 |
| Example 5 | Compound 83 | 3.59 | 55.06 | 0.680, 0.320 | 397 |
| Example 6 | Compound 123 | 3.55 | 55.81 | 0.680, 0.320 | 392 |
| Example 7 | Compound 125 | 3.41 | 56.53 | 0.680, 0.320 | 394 |
| Example 8 | Compound 160 | 3.44 | 55.11 | 0.680, 0.320 | 393 |
| Example 9 | Compound 175 | 3.54 | 55.28 | 0.680, 0.320 | 403 |
| Example 10 | Compound 178 | 3.60 | 56.55 | 0.680, 0.320 | 401 |
| Example 11 | Compound 188 | 3.56 | 55.74 | 0.680, 0.320 | 396 |
| Example 12 | Compound 189 | 3.43 | 55.18 | 0.680, 0.320 | 391 |
| Example 13 | Compound 212 | 3.65 | 55.62 | 0.680, 0.320 | 405 |
| Example 14 | Compound 215 | 3.52 | 55.01 | 0.680, 0.320 | 400 |
| Example 15 | Compound 248 | 3.58 | 56.68 | 0.680, 0.320 | 441 |
| Example 16 | Compound 251 | 3.46 | 55.43 | 0.680, 0.320 | 444 |
| Example 17 | Compound 269 | 3.48 | 56.07 | 0.680, 0.320 | 451 |
| Example 18 | Compound 270 | 3.63 | 56.73 | 0.680, 0.320 | 438 |
| Example 19 | Compound 271 | 3.45 | 56.64 | 0.680, 0.320 | 445 |
| Example 20 | Compound 282 | 3.36 | 55.13 | 0.680, 0.320 | 452 |
| Example 21 | Compound 345 | 3.65 | 55.37 | 0.680, 0.320 | 447 |
| Example 22 | Compound 351 | 3.51 | 55.77 | 0.680, 0.320 | 446 |
| Example 23 | Compound 358 | 3.39 | 55.93 | 0.680, 0.320 | 443 |
| Example 24 | Compound 361 | 3.37 | 55.91 | 0.680, 0.320 | 448 |
| Example 25 | Compound 363 | 3.35 | 55.83 | 0.680, 0.320 | 449 |
| Example 26 | Compound 364 | 3.64 | 56.18 | 0.680, 0.320 | 450 |
| Example 27 | Compound 418 | 3.40 | 56.35 | 0.680, 0.320 | 404 |
| Example 28 | Compound 445 | 3.39 | 55.98 | 0.680, 0.320 | 390 |
| Example 29 | Compound 483 | 3.35 | 56.23 | 0.680, 0.320 | 440 |
| Example 30 | Compound 487 | 3.62 | 55.76 | 0.680, 0.320 | 442 |
| Comparative | Compound A | 3.53 | 47.64 | 0.680, 0.320 | 332 |
| Example 1 | | | | | |
| Comparative | Compound B | 3.42 | 48.17 | 0.680, 0.320 | 346 |
| Example 2 | | | | | |
| Comparative | Compound C | 3.47 | 46.38 | 0.680, 0.320 | 327 |
| Example 3 | | | | | |
| Comparative | Compound D | 3.61 | 45.93 | 0.680, 0.320 | 315 |
| Example 4 | | | | | |

According to the results in Table 8, it can be seen that, Examples 1 to 30 using the organic compounds of the present invention as the hole transport type material in the mixed host material of red organic electroluminescent devices showed an increase in current efficiency of at least 14.2% and an increase in lifetime of at least 12.7% compared to Comparative Examples 1 to 4 corresponding to the devices using the known compounds.

### Example 31: Red Organic Electroluminescent Device

The anode was prepared through the following process: An ITO/Ag/ITO substrate with thicknesses of 100 Å, 1000 Å, and 100 Å in order was cut into a size of 40 mm (length)×40 mm (width)×0.7 mm (height), and prepared into a test substrate having anode and insulation layer patterns with photolithography process. Ultraviolet ozone and O₂:N₂ plasma were used to perform surface treatment to increase the work function of the anode, and the surface of ITO substrates was cleaned using organic solvents to remove impurities and oil stains.

PD and HT-36 were co-vapor deposited on the test substrate (anode) at a vapor deposition rate ratio of 2%:98% to form a hole injection layer with a thickness of 100 Å.

The compound HT-36 was vacuum vapor deposited on the hole injection layer to form a first hole transport layer with a thickness of 1065 Å.

The compound 21 was vacuum vapor deposited on the first hole transport layer to form a second hole transport layer with a thickness of 890 Å.

RH-P: RH-N: RD were co-vapor deposited on the second hole transport layer at a vapor deposition rate ratio of 49%:49%:2% to form an organic light-emitting layer with a thickness of 400 Å.

Compound ET-21 and LiQ were co-vapor deposited on the organic light-emitting layer at a vapor deposition rate ratio of 1:1 to form an electron transport layer (ETL) with a thickness of 350 Å. Yb was vapor deposited on the electron transport layer to form an electron injection layer with a thickness of 10 Å. Then, magnesium (Mg) and silver (Ag) were mixed at a vapor deposition rate of 1:9, and vacuum vapor deposited on the electron injection layer to form a cathode with a thickness of 130 Å.

In addition, Compound CP-1 was vacuum vapor deposited on the above cathode to form an organic capping layer with a thickness of 800 Å, thus completing the fabrication of the red organic electroluminescent device.

### Examples 32 to 59

Organic electroluminescent devices were fabricated by the same method as used in Example 31, except that Compound listed in table 10 below was used instead of the Compound 21 in Example 31 when forming the second hole transport layer.

### Comparative Examples 5 to 8

Organic electroluminescent devices were fabricated by the same method as used in Example 31, except that Compound E, Compound F, Compound G, and Compound H were used respectively instead of the Compound 21 in Example 31 when forming the second hole transport layer.

Among them, the compounds used to fabricate the devices of each Example and Comparative Example are listed in Table 9 below.

TABLE 9 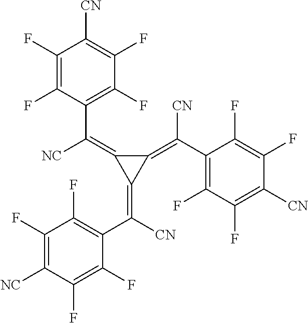 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]
PD 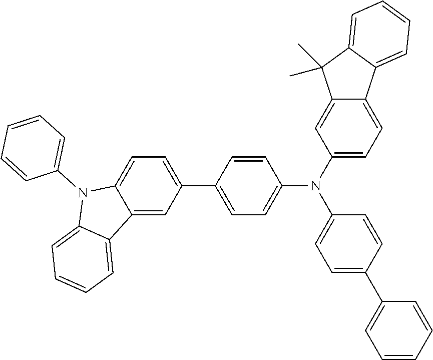 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]
HT-36 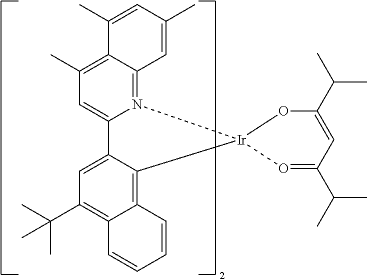
RD 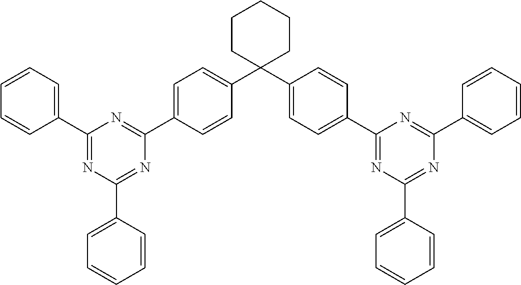 [SMILES: c1ccc(-c2nc(-c3ccccc3)nc(-c3ccc(C4(c5ccc(-c6nc(-c7ccccc7)nc(-c7ccccc7)n6)cc5)CCCCC4)cc3)n2)cc1]
ET-21 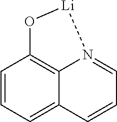
LiQ 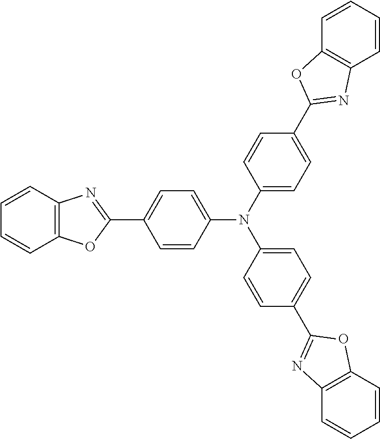 [SMILES: c1ccc2oc(-c3ccc(N(c4ccc(-c5nc6ccccc6o5)cc4)c4ccc(-c5nc6ccccc6o5)cc4)cc3)nc2c1]
CP-1 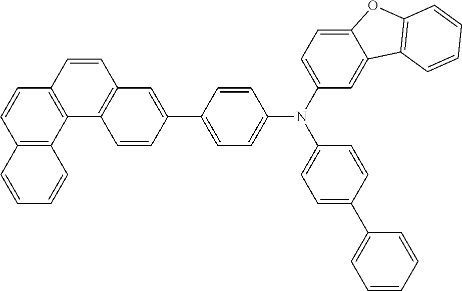 [SMILES: c1ccc(-c2ccc(N(c3ccc(-c4ccc5c(ccc6ccc7ccccc7c65)c4)cc3)c3ccc4oc5ccccc5c4c3)cc2)cc1]
RH-P 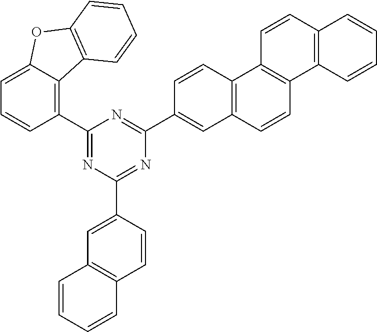 [SMILES: c1ccc2cc(-c3nc(-c4ccc5c(ccc6c7ccccc7ccc56)c4)nc(-c4cccc5oc6ccccc6c45)n3)ccc2c1]
RH-N 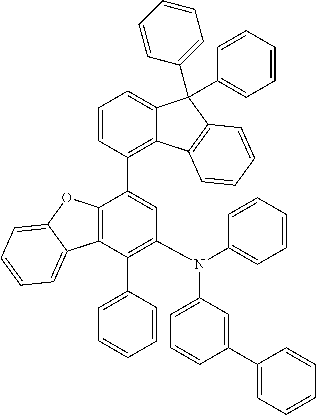 [SMILES: c1ccc(-c2cccc(N(c3ccccc3)c3cc(-c4cccc5c4-c4ccccc4C5(c4ccccc4)c4ccccc4)c4oc5ccccc5c4c3-c3ccccc3)c2)cc1]
Compound E 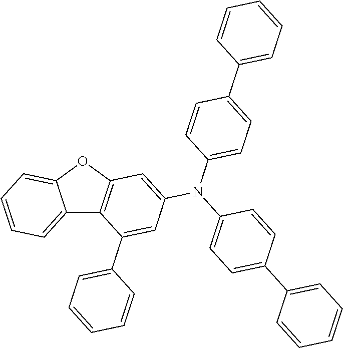 [SMILES: c1ccc(-c2ccc(N(c3ccc(-c4ccccc4)cc3)c3cc(-c4ccccc4)c4c(c3)oc3ccccc34)cc2)cc1]
Compound F 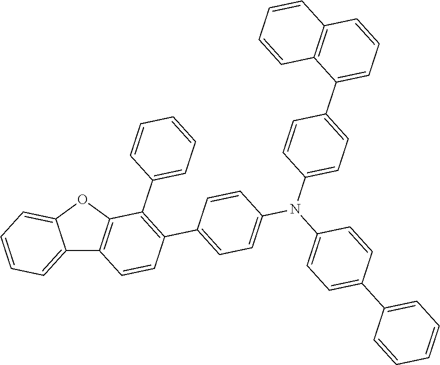 [SMILES: c1ccc(-c2ccc(N(c3ccc(-c4ccc5c(oc6ccccc65)c4-c4ccccc4)cc3)c3ccc(-c4cccc5ccccc45)cc3)cc2)cc1]
Compound G 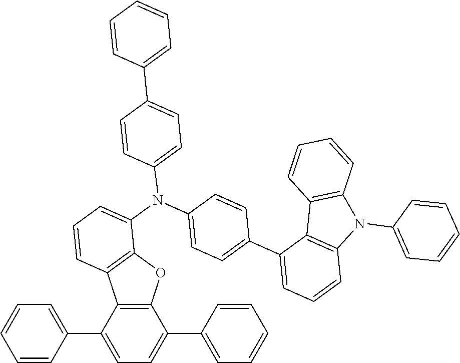 [SMILES: c1ccc(-c2ccc(N(c3ccc(-c4cccc5c4c4ccccc4n5-c4ccccc4)cc3)c3cccc4c3oc3c(-c5ccccc5)ccc(-c5ccccc5)c34)cc2)cc1]
Compound H

Performance tests were conducted on the red organic electroluminescent devices fabricated in Examples 31 to 59 and Comparative Examples 5 to 8. IVL performance (driving voltage, current efficiency, color coordinates) was tested at 10 mA/cm², and T95 device lifetime was tested at 20 mA/cm². The results are shown in Table 10.

TABLE 10

| | Second Hole | Driving | Current | Color | T95 |
|---|---|---|---|---|---|
| Example | Transport | Voltage | Efficiency | Coordinates | Lifetime |
| No. | Layer; | (V) | (Cd/A) | CIEx, CIEy | (hrs) |
| Example 31 | Compound 21 | 3.43 | 56.17 | 0.680, 0.320 | 443 |
| Example 32 | Compound 25 | 3.41 | 56.23 | 0.680, 0.320 | 442 |
| Example 33 | Compound 28 | 3.45 | 55.92 | 0.680, 0.320 | 437 |
| Example 34 | Compound 29 | 3.36 | 55.63 | 0.680, 0.320 | 439 |
| Example 35 | Compound 37 | 3.44 | 56.07 | 0.680, 0.320 | 447 |
| Example 36 | Compound 44 | 3.42 | 56.38 | 0.680, 0.320 | 435 |
| Example 37 | Compound 50 | 3.43 | 56.42 | 0.680, 0.320 | 438 |
| Example 38 | Compound 52 | 3.38 | 56.57 | 0.680, 0.320 | 444 |
| Example 39 | Compound 57 | 3.47 | 55.78 | 0.680, 0.320 | 440 |
| Example 40 | Compound 68 | 3.42 | 56.14 | 0.680, 0.320 | 442 |
| Example 41 | Compound 71 | 3.41 | 55.73 | 0.680, 0.320 | 438 |
| Example 42 | Compound 74 | 3.36 | 56.22 | 0.680, 0.320 | 443 |
| Example 43 | Compound 89 | 3.46 | 55.89 | 0.680, 0.320 | 447 |
| Example 44 | Compound 108 | 3.37 | 55.61 | 0.680, 0.320 | 436 |
| Example 45 | Compound 155 | 3.46 | 55.66 | 0.680, 0.320 | 441 |
| Example 46 | Compound 163 | 3.40 | 55.83 | 0.680, 0.320 | 445 |
| Example 47 | Compound 164 | 3.37 | 56.49 | 0.680, 0.320 | 448 |
| Example 48 | Compound 167 | 3.40 | 56.01 | 0.680, 0.320 | 439 |
| Example 49 | Compound 173 | 3.38 | 56.37 | 0.680, 0.320 | 435 |
| Example 50 | Compound 179 | 3.39 | 55.97 | 0.680, 0.320 | 440 |
| Example 51 | Compound 259 | 3.21 | 59.73 | 0.680, 0.320 | 449 |
| Example 52 | Compound 261 | 3.24 | 59.21 | 0.680, 0.320 | 445 |
| Example 53 | Compound 267 | 3.25 | 59.55 | 0.680, 0.320 | 450 |
| Example 54 | Compound 289 | 3.22 | 58.94 | 0.680, 0.320 | 437 |
| Example 55 | Compound 304 | 3.23 | 59.46 | 0.680, 0.320 | 448 |
| Example 56 | Compound 325 | 3.24 | 59.67 | 0.680, 0.320 | 446 |
| Example 57 | Compound 339 | 3.23 | 59.83 | 0.680, 0.320 | 444 |
| Example 58 | Compound 475 | 3.25 | 59.32 | 0.680, 0.320 | 441 |
| Example 59 | Compound 482 | 3.22 | 59.08 | 0.680, 0.320 | 446 |
| Comparative | Compound E | 3.48 | 49.15 | 0.680, 0.320 | 381 |
| Example 5 | | | | | |
| Comparative | Compound F | 3.44 | 47.73 | 0.680, 0.320 | 373 |
| Example 6 | | | | | |
| Comparative | Compound G | 3.45 | 50.64 | 0.680, 0.320 | 392 |
| Example 7 | | | | | |
| Comparative | Compound H | 3.39 | 45.56 | 0.680, 0.320 | 367 |
| Example 8 | | | | | |

According to the results in Table 10, it can be seen that, Examples 31 to 59 using the organic compounds of the present invention as the second hole transport material in the red organic electroluminescent devices showed an increase in current efficiency of at least 10% and an increase in lifetime of at least 11% compared to Comparative Examples 5 to 8 corresponding to the devices using the known compounds.

The preferred embodiments of the present disclosure are described in detail above in conjunction with the accompanying drawings. However, the present disclosure is not limited to the specific details of the above embodiments. Within the scope of the technical concept of the present disclosure, various simple modifications can be made to the technical solutions of the present disclosure, and all these simple modifications fall within the protection scope of the present disclosure.

Furthermore, it should be noted that the specific technical features described in the above embodiments can be combined in any suitable way without contradiction. To avoid unnecessary repetition, the present disclosure will not separately explain various possible combination methods.

1. An organic compound, having a structure shown in a Formula I: 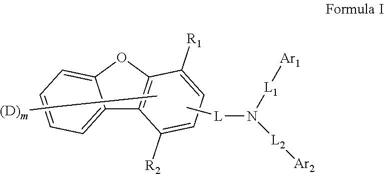
wherein, R₁ and R₂ are the same or different, and are each independently a substituted or unsubstituted aryl having 6 to 12 carbon atoms;
the substituent(s) of R₁ and R₂ are the same or different, and are each independently selected from a deuterium, a halogen group, a cyano, or an alkyl having 1 to 5 carbon atoms;
L, L₁, and L₂ are the same or different, and are each independently selected from a single bond, a substituted or unsubstituted arylene having 6 to 30 carbon atoms, or a substituted or unsubstituted heteroarylene having 3 to 30 carbon atoms;
Ar₁ and Ar₂ are the same or different, and are each independently selected from a substituted or unsubstituted aryl having 6 to 30 carbon atoms, or a substituted or unsubstituted heteroaryl having 3 to 30 carbon atoms;
the substituent(s) of L, L₁, L₂, Ar₁ and Ar₂ are the same or different, and are each independently selected from a deuterium, a halogen group, a cyano, an alkyl having 1 to 10 carbon atoms, a deuteroalkyl having 1 to 10 carbon atoms, a trialkylsilyl having 3 to 12 carbon atoms, a haloalkyl having 1 to 10 carbon atoms, a cycloalkyl having 3 to 10 carbon atoms, an aryl having 6 to 20 carbon atoms, or a heteroaryl having 3 to 20 carbon atoms;
m represents the number of D, and m is selected from 0, 1, 2, 3, 4, or 5.

2. The organic compound according to claim 1, wherein the organic compound is selected from the structure represented by a Formula I-I or a Formula I-II:

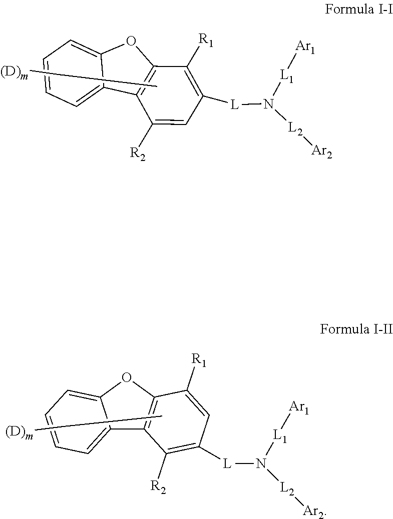

3. The organic compound according to claim 1, wherein Ar₁ and Ar₂ are each independently selected from a substituted or unsubstituted aryl having 6 to 21 carbon atoms; the substituent(s) of Ar₁ and Ar₂ are each independently selected from a deuterium, a fluoro, a cyano, a trimethylsilyl, an alkyl having 1 to 5 carbon atoms, a haloalkyl having 1 to 5 carbon atoms, a deuteroalkyl having 1 to 5 carbon atoms, or an aryl having 6 to 12 carbon atoms;
preferably, Ar₁ and Ar₂ are each independently selected from a substituted or unsubstituted phenyl, a substituted or unsubstituted naphthyl, a substituted or unsubstituted biphenyl, a substituted or unsubstituted terphenyl, or a substituted or unsubstituted fluorenyl;
optionally, the substituent(s) of Ar₁ and Ar₂ are each independently selected from a deuterium, a fluoro, a cyano, a trimethylsilyl, a trifluoromethyl, a methyl, an ethyl, an isopropyl, a tert-butyl, a trideuteromethyl, or a phenyl.

4. The organic compound according to claim 1, wherein R₁ and R₂ are each independently selected from a substituted or unsubstituted phenyl, a substituted or unsubstituted naphthyl, an unsubstituted biphenyl, or a deuterium-substituted biphenyl;
optionally, the substituent(s) of R₁ and R₂ are each independently selected from a deuterium, a fluoro, a cyano, a methyl, an ethyl, an isopropyl, or a tert-butyl.

5. The organic compound according to claim 1, wherein L, L₁ and L₂ are each independently selected from a single bond, a substituted or unsubstituted phenylene, a substituted or unsubstituted naphthylene, a substituted or unsubstituted biphenylene, or a substituted or unsubstituted fluorenylene;
optionally, the substituent(s) of L, L₁ and L₂ are each independently selected from a deuterium, a fluoro, a cyano, a trifluoromethyl, a trimethylsilyl, a methyl, a ethyl, an isopropyl, a tert-butyl, or a phenyl.

6. The organic compound according to claim 1, wherein L is selected from a single bond or the following groups: 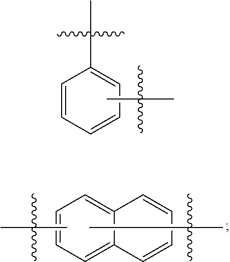
optionally, L₁ and L₂ are each independently selected from a single bond, or the following groups:

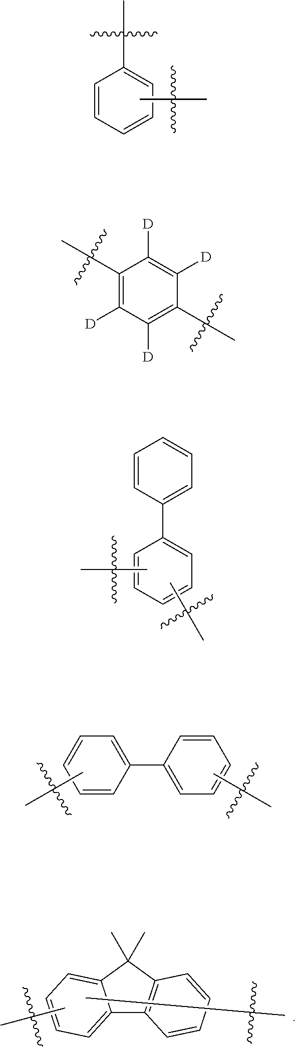

7. The organic compound according to claim 1, wherein Ar₁ and Ar₂ are each independently selected from the group consisting of the following groups:

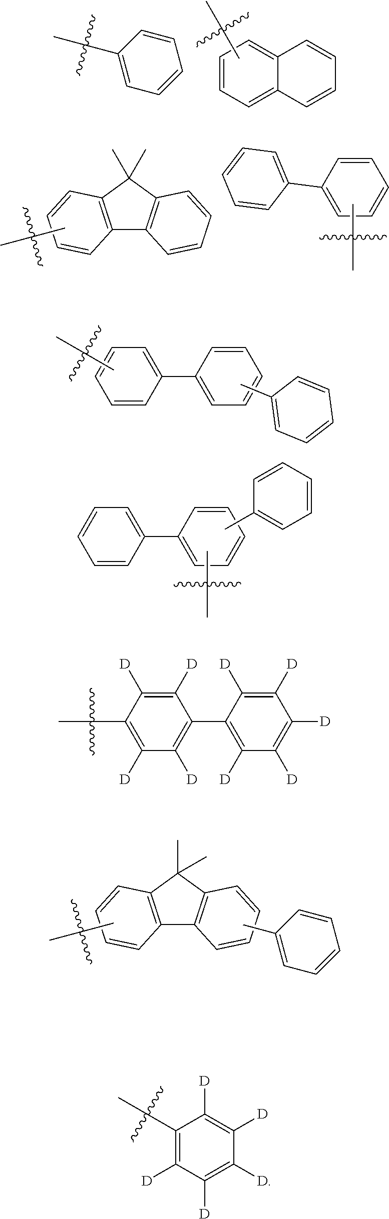

8. The organic compound according to claim 1, wherein 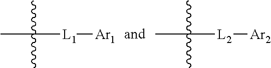
are each independently selected from the group consisting of the following groups:

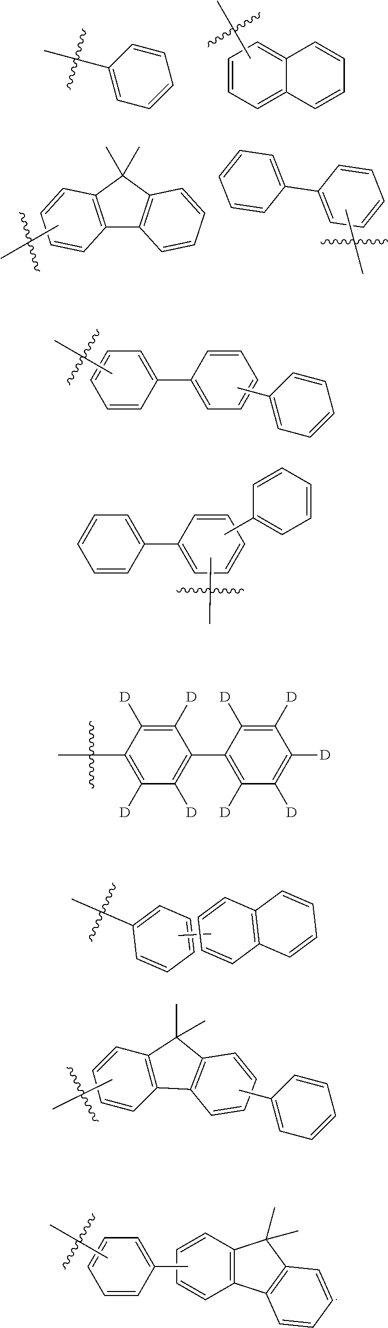

9. The organic compound according to claim 1, wherein R₁ and R₂ are each independently selected from the group consisting of the following groups:

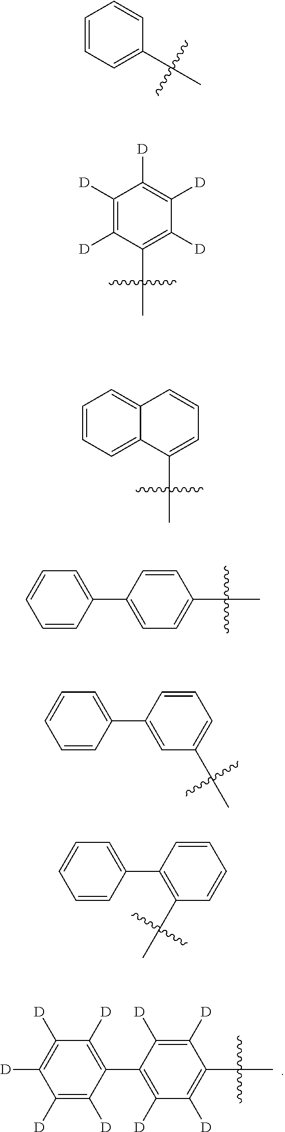

10. The organic compound according to claim 1, wherein the organic compound is selected from the group consisting of the following groups:

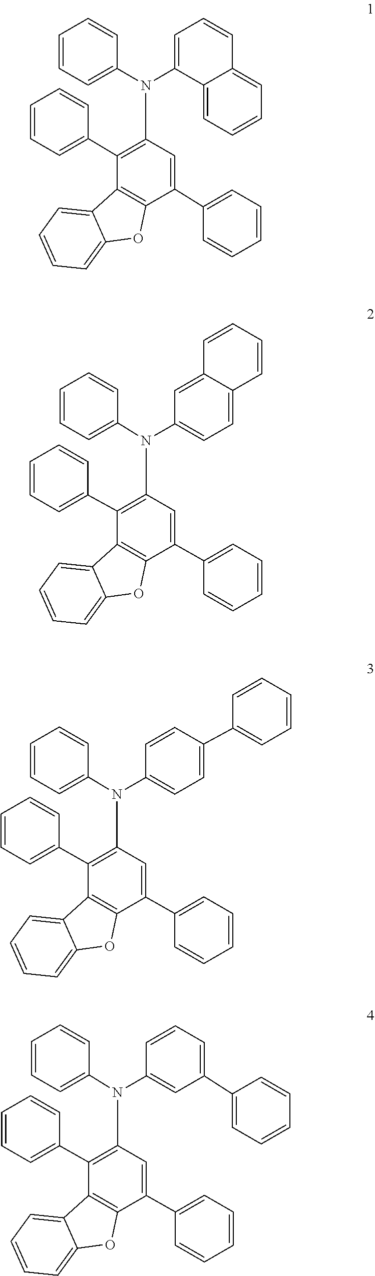 [SMILES: c1ccc(-c2cc(N(c3ccccc3)c3cccc4ccccc34)c(-c3ccccc3)c3c2oc2ccccc23)cc1 ; c1ccc(-c2cc(N(c3ccccc3)c3ccc4ccccc4c3)c(-c3ccccc3)c3c2oc2ccccc23)cc1 ; c1ccc(-c2ccc(N(c3ccccc3)c3cc(-c4ccccc4)c4oc5ccccc5c4c3-c3ccccc3)cc2)cc1 ; c1ccc(-c2cccc(N(c3ccccc3)c3cc(-c4ccccc4)c4oc5ccccc5c4c3-c3ccccc3)c2)cc1]

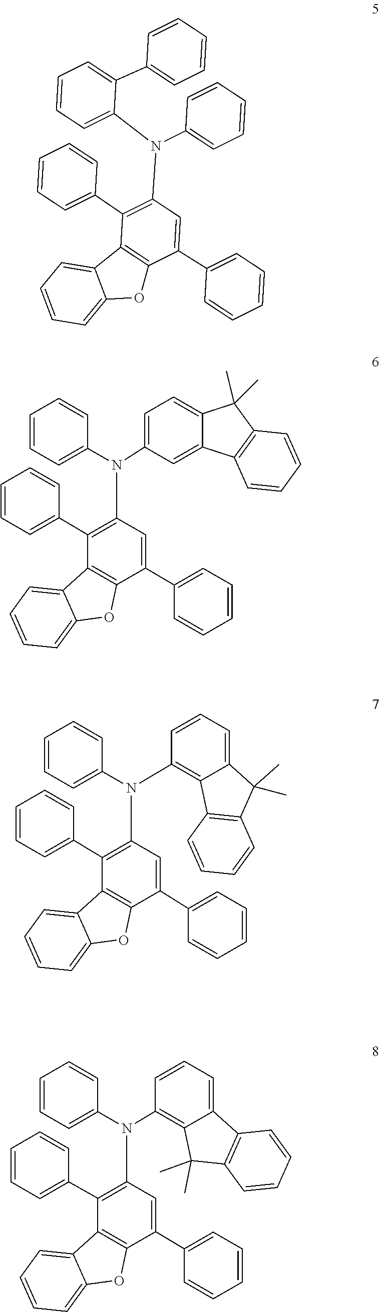 [SMILES: c1ccc(-c2ccccc2N(c2ccccc2)c2cc(-c3ccccc3)c3oc4ccccc4c3c2-c2ccccc2)cc1 ; CC1(C)c2ccccc2-c2cc(N(c3ccccc3)c3cc(-c4ccccc4)c4oc5ccccc5c4c3-c3ccccc3)ccc21 ; CC1(C)c2ccccc2-c2c(N(c3ccccc3)c3cc(-c4ccccc4)c4oc5ccccc5c4c3-c3ccccc3)cccc21 ; CC1(C)c2ccccc2-c2cccc(N(c3ccccc3)c3cc(-c4ccccc4)c4oc5ccccc5c4c3-c3ccccc3)c21]

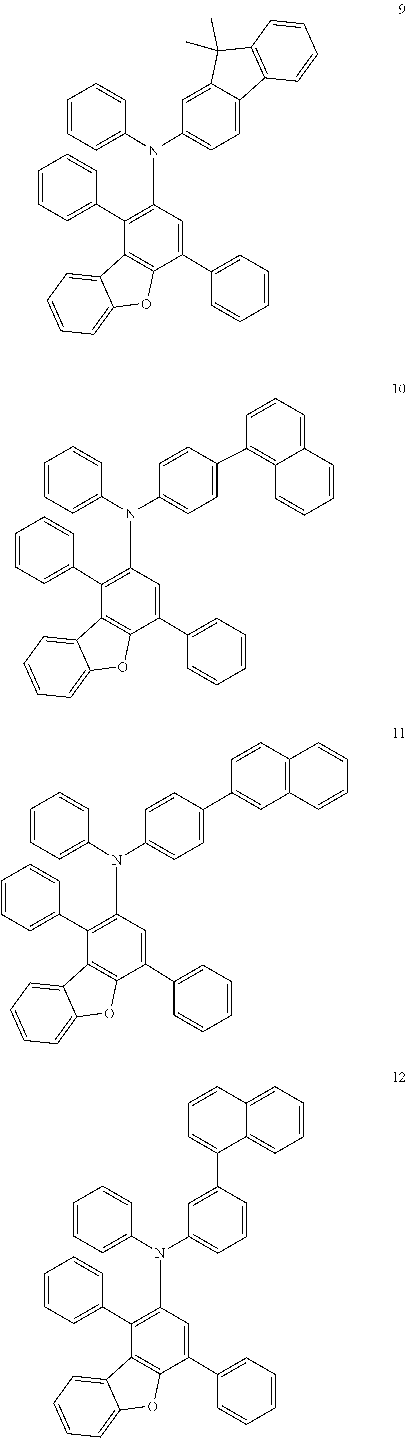 [SMILES: CC1(C)c2ccccc2-c2ccc(N(c3ccccc3)c3cc(-c4ccccc4)c4oc5ccccc5c4c3-c3ccccc3)cc21 ; c1ccc(-c2cc(N(c3ccccc3)c3ccc(-c4cccc5ccccc45)cc3)c(-c3ccccc3)c3c2oc2ccccc23)cc1 ; c1ccc(-c2cc(N(c3ccccc3)c3ccc(-c4ccc5ccccc5c4)cc3)c(-c3ccccc3)c3c2oc2ccccc23)cc1 ; c1ccc(-c2cc(N(c3ccccc3)c3cccc(-c4cccc5ccccc45)c3)c(-c3ccccc3)c3c2oc2ccccc23)cc1]

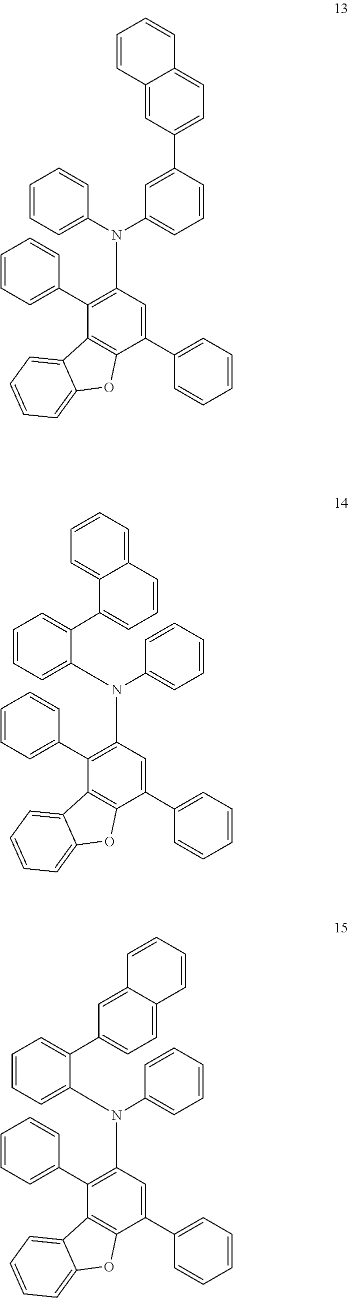 [SMILES: c1ccc(-c2cc(N(c3ccccc3)c3cccc(-c4ccc5ccccc5c4)c3)c(-c3ccccc3)c3c2oc2ccccc23)cc1 ; c1ccc(-c2cc(N(c3ccccc3)c3ccccc3-c3cccc4ccccc34)c(-c3ccccc3)c3c2oc2ccccc23)cc1 ; c1ccc(-c2cc(N(c3ccccc3)c3ccccc3-c3ccc4ccccc4c3)c(-c3ccccc3)c3c2oc2ccccc23)cc1]

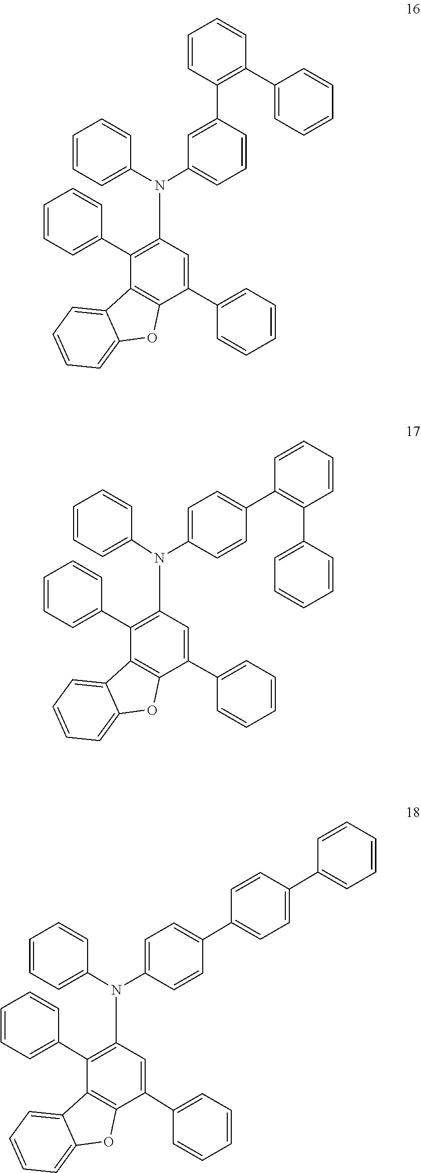 [SMILES: c1ccc(-c2ccccc2-c2cccc(N(c3ccccc3)c3cc(-c4ccccc4)c4oc5ccccc5c4c3-c3ccccc3)c2)cc1 ; c1ccc(-c2ccccc2-c2ccc(N(c3ccccc3)c3cc(-c4ccccc4)c4oc5ccccc5c4c3-c3ccccc3)cc2)cc1 ; c1ccc(-c2ccc(-c3ccc(N(c4ccccc4)c4cc(-c5ccccc5)c5oc6ccccc6c5c4-c4ccccc4)cc3)cc2)cc1]

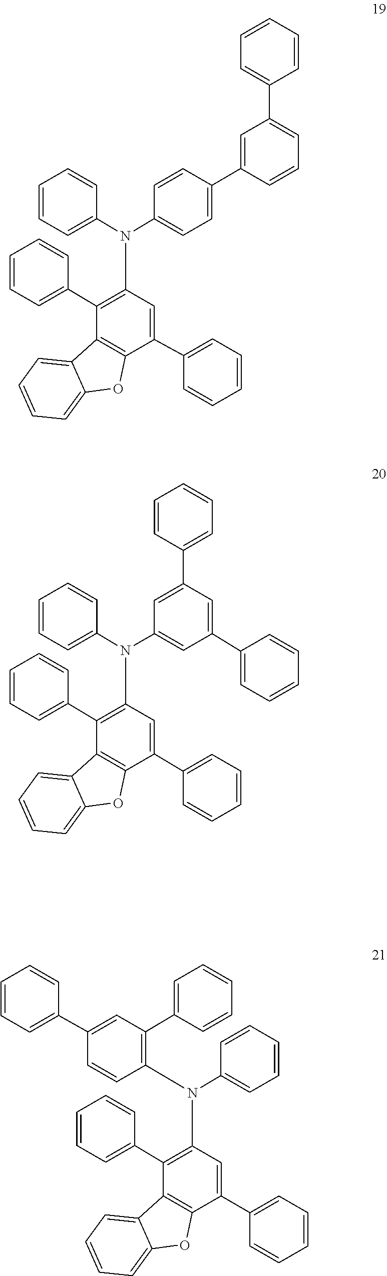 [SMILES: c1ccc(-c2cccc(-c3ccc(N(c4ccccc4)c4cc(-c5ccccc5)c5oc6ccccc6c5c4-c4ccccc4)cc3)c2)cc1 ; c1ccc(-c2cc(-c3ccccc3)cc(N(c3ccccc3)c3cc(-c4ccccc4)c4oc5ccccc5c4c3-c3ccccc3)c2)cc1 ; c1ccc(-c2ccc(N(c3ccccc3)c3cc(-c4ccccc4)c4oc5ccccc5c4c3-c3ccccc3)c(-c3ccccc3)c2)cc1]

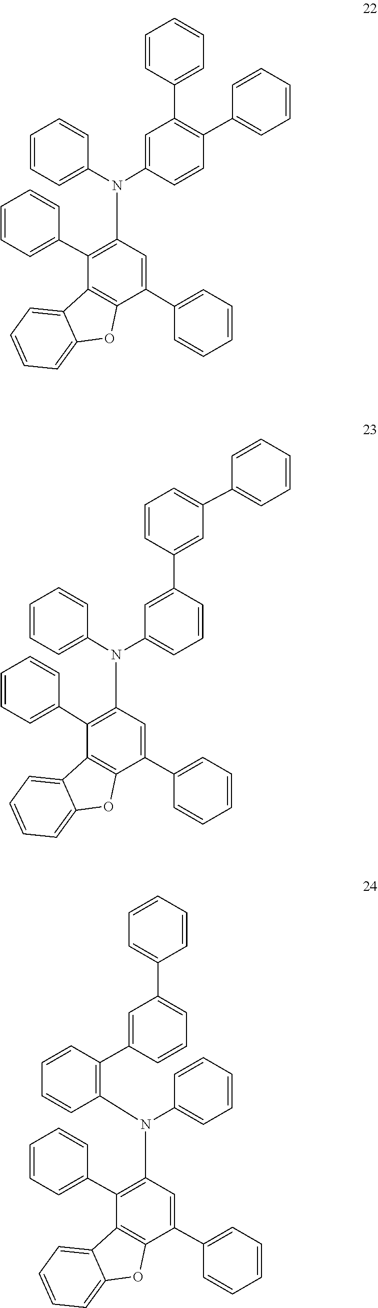 [SMILES: c1ccc(-c2ccc(N(c3ccccc3)c3cc(-c4ccccc4)c4oc5ccccc5c4c3-c3ccccc3)cc2-c2ccccc2)cc1 ; c1ccc(-c2cccc(-c3cccc(N(c4ccccc4)c4cc(-c5ccccc5)c5oc6ccccc6c5c4-c4ccccc4)c3)c2)cc1 ; c1ccc(-c2cccc(-c3ccccc3N(c3ccccc3)c3cc(-c4ccccc4)c4oc5ccccc5c4c3-c3ccccc3)c2)cc1]

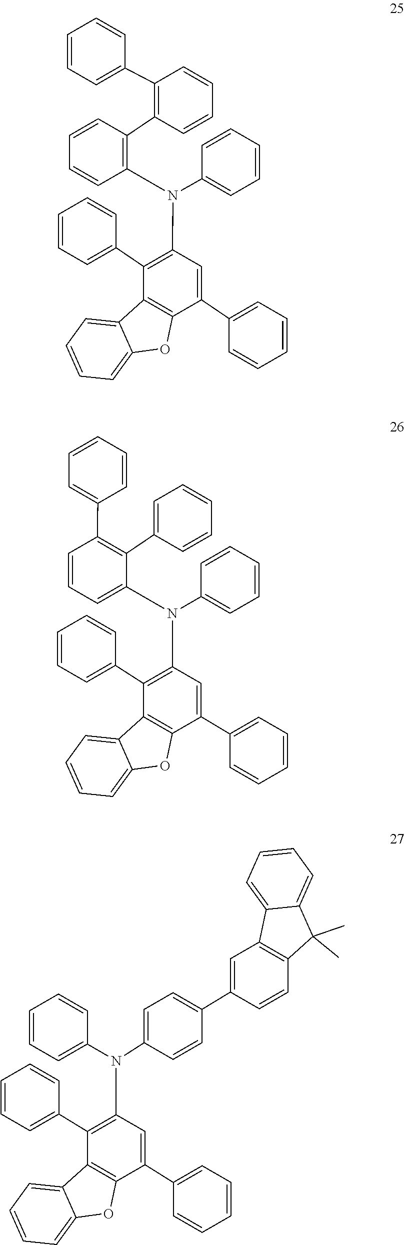 [SMILES: c1ccc(-c2ccccc2-c2ccccc2N(c2ccccc2)c2cc(-c3ccccc3)c3oc4ccccc4c3c2-c2ccccc2)cc1 ; c1ccc(-c2cccc(N(c3ccccc3)c3cc(-c4ccccc4)c4oc5ccccc5c4c3-c3ccccc3)c2-c2ccccc2)cc1 ; CC1(C)c2ccccc2-c2cc(-c3ccc(N(c4ccccc4)c4cc(-c5ccccc5)c5oc6ccccc6c5c4-c4ccccc4)cc3)ccc21]

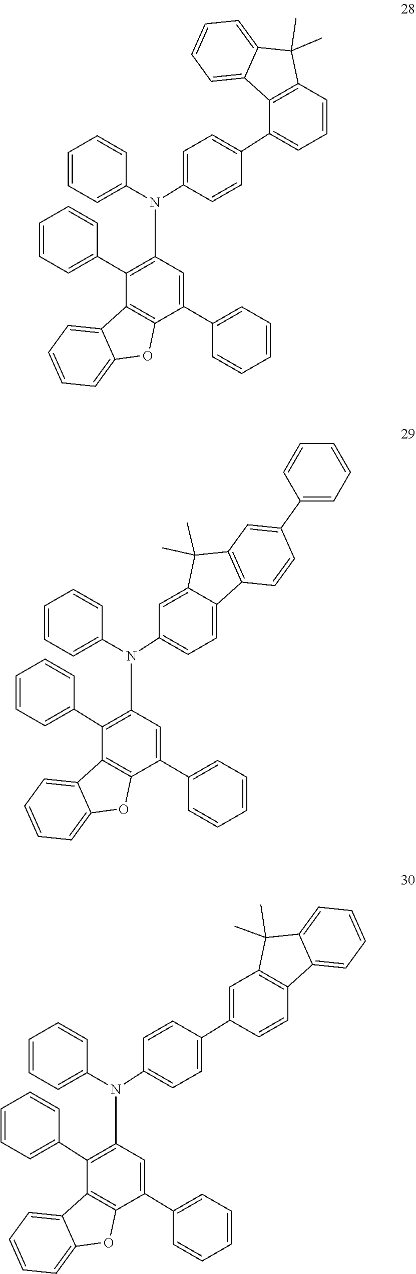 [SMILES: CC1(C)c2ccccc2-c2c(-c3ccc(N(c4ccccc4)c4cc(-c5ccccc5)c5oc6ccccc6c5c4-c4ccccc4)cc3)cccc21 ; CC1(C)c2cc(-c3ccccc3)ccc2-c2ccc(N(c3ccccc3)c3cc(-c4ccccc4)c4oc5ccccc5c4c3-c3ccccc3)cc21 ; CC1(C)c2ccccc2-c2ccc(-c3ccc(N(c4ccccc4)c4cc(-c5ccccc5)c5oc6ccccc6c5c4-c4ccccc4)cc3)cc21]

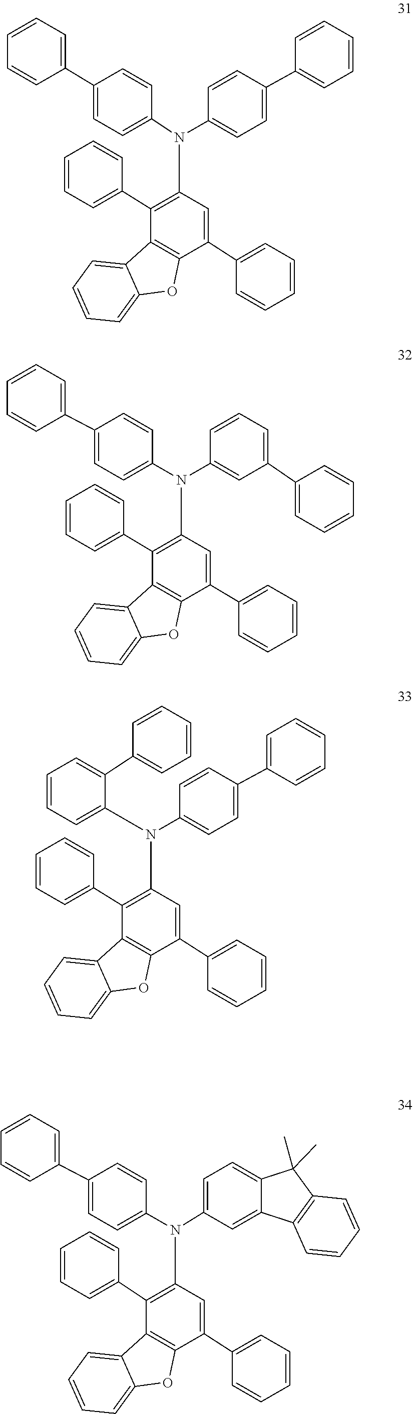 [SMILES: c1ccc(-c2ccc(N(c3ccc(-c4ccccc4)cc3)c3cc(-c4ccccc4)c4oc5ccccc5c4c3-c3ccccc3)cc2)cc1 ; c1ccc(-c2ccc(N(c3cccc(-c4ccccc4)c3)c3cc(-c4ccccc4)c4oc5ccccc5c4c3-c3ccccc3)cc2)cc1 ; c1ccc(-c2ccc(N(c3ccccc3-c3ccccc3)c3cc(-c4ccccc4)c4oc5ccccc5c4c3-c3ccccc3)cc2)cc1 ; CC1(C)c2ccccc2-c2cc(N(c3ccc(-c4ccccc4)cc3)c3cc(-c4ccccc4)c4oc5ccccc5c4c3-c3ccccc3)ccc21]

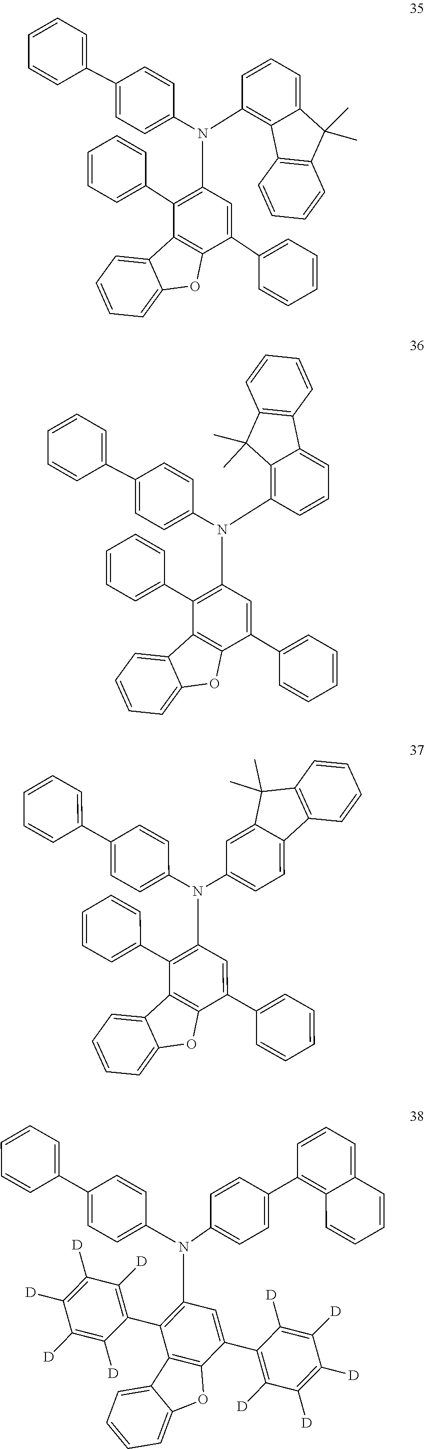 [SMILES: CC1(C)c2ccccc2-c2c(N(c3ccc(-c4ccccc4)cc3)c3cc(-c4ccccc4)c4oc5ccccc5c4c3-c3ccccc3)cccc21 ; CC1(C)c2ccccc2-c2cccc(N(c3ccc(-c4ccccc4)cc3)c3cc(-c4ccccc4)c4oc5ccccc5c4c3-c3ccccc3)c21 ; CC1(C)c2ccccc2-c2ccc(N(c3ccc(-c4ccccc4)cc3)c3cc(-c4ccccc4)c4oc5ccccc5c4c3-c3ccccc3)cc21 ; [2H]c1c([2H])c([2H])c(-c2cc(N(c3ccc(-c4ccccc4)cc3)c3ccc(-c4cccc5ccccc45)cc3)c(-c3c([2H])c([2H])c([2H])c([2H])c3[2H])c3c2oc2ccccc23)c([2H])c1[2H]]

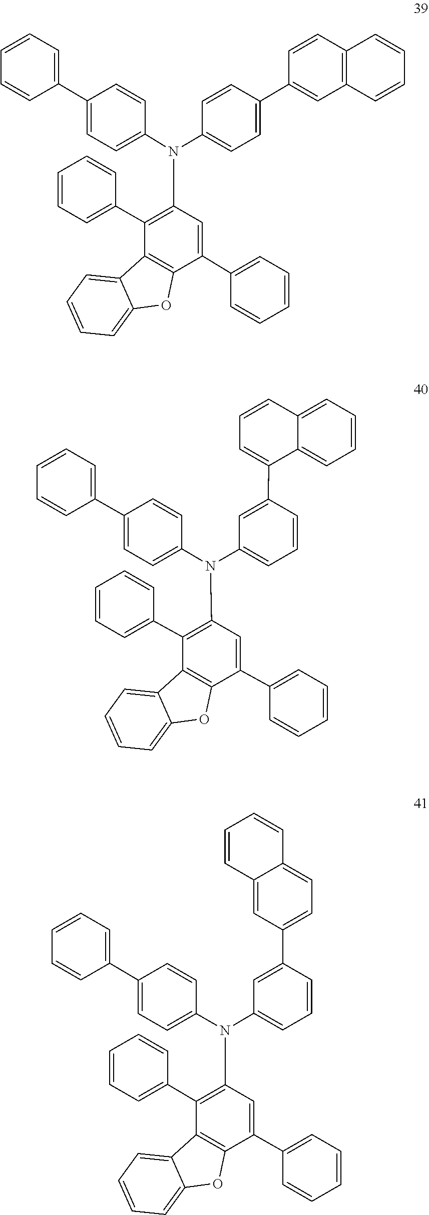 [SMILES: c1ccc(-c2ccc(N(c3ccc(-c4ccc5ccccc5c4)cc3)c3cc(-c4ccccc4)c4oc5ccccc5c4c3-c3ccccc3)cc2)cc1 ; c1ccc(-c2ccc(N(c3cccc(-c4cccc5ccccc45)c3)c3cc(-c4ccccc4)c4oc5ccccc5c4c3-c3ccccc3)cc2)cc1 ; c1ccc(-c2ccc(N(c3cccc(-c4ccc5ccccc5c4)c3)c3cc(-c4ccccc4)c4oc5ccccc5c4c3-c3ccccc3)cc2)cc1]

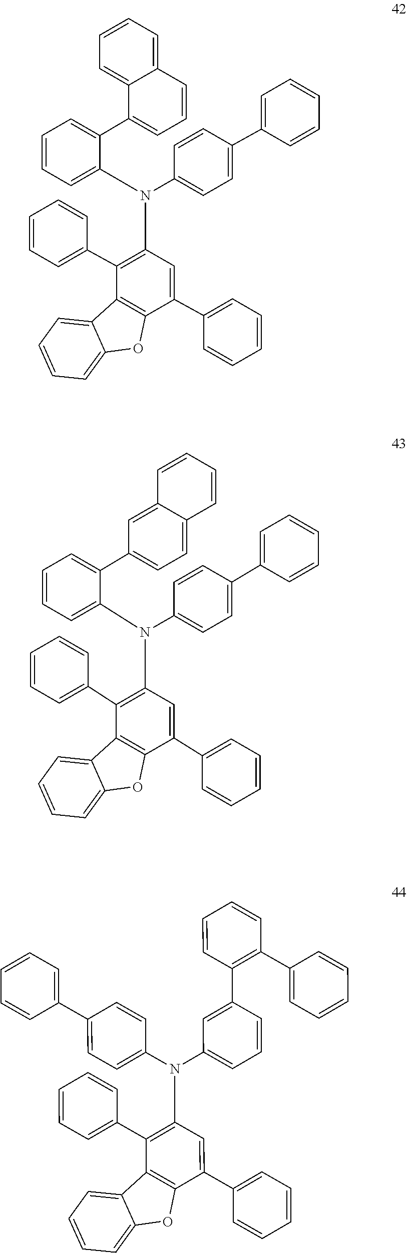 [SMILES: c1ccc(-c2ccc(N(c3ccccc3-c3cccc4ccccc34)c3cc(-c4ccccc4)c4oc5ccccc5c4c3-c3ccccc3)cc2)cc1 ; c1ccc(-c2ccc(N(c3ccccc3-c3ccc4ccccc4c3)c3cc(-c4ccccc4)c4oc5ccccc5c4c3-c3ccccc3)cc2)cc1 ; c1ccc(-c2ccc(N(c3cccc(-c4ccccc4-c4ccccc4)c3)c3cc(-c4ccccc4)c4oc5ccccc5c4c3-c3ccccc3)cc2)cc1]

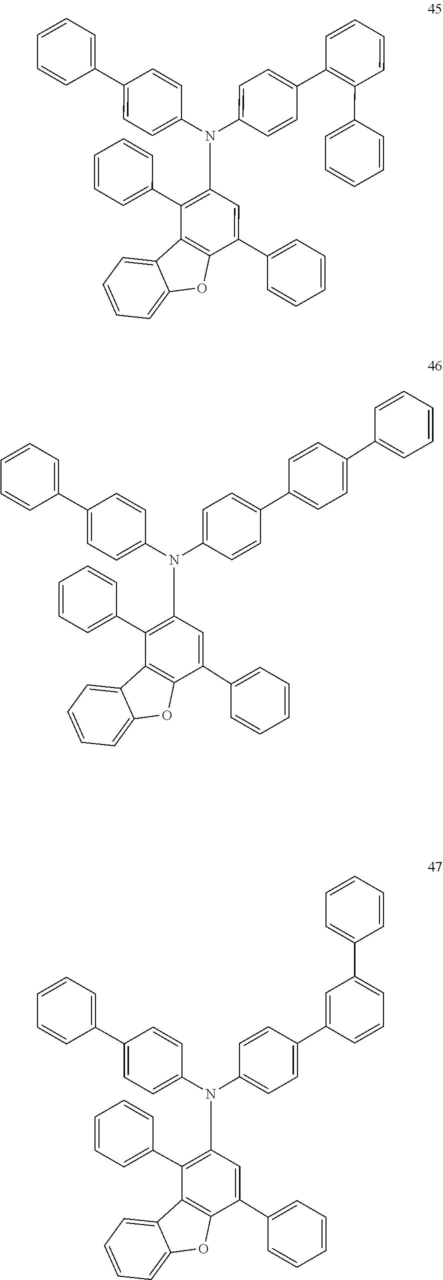 [SMILES: c1ccc(-c2ccc(N(c3ccc(-c4ccccc4-c4ccccc4)cc3)c3cc(-c4ccccc4)c4oc5ccccc5c4c3-c3ccccc3)cc2)cc1 ; c1ccc(-c2ccc(-c3ccc(N(c4ccc(-c5ccccc5)cc4)c4cc(-c5ccccc5)c5oc6ccccc6c5c4-c4ccccc4)cc3)cc2)cc1 ; c1ccc(-c2ccc(N(c3ccc(-c4cccc(-c5ccccc5)c4)cc3)c3cc(-c4ccccc4)c4oc5ccccc5c4c3-c3ccccc3)cc2)cc1]

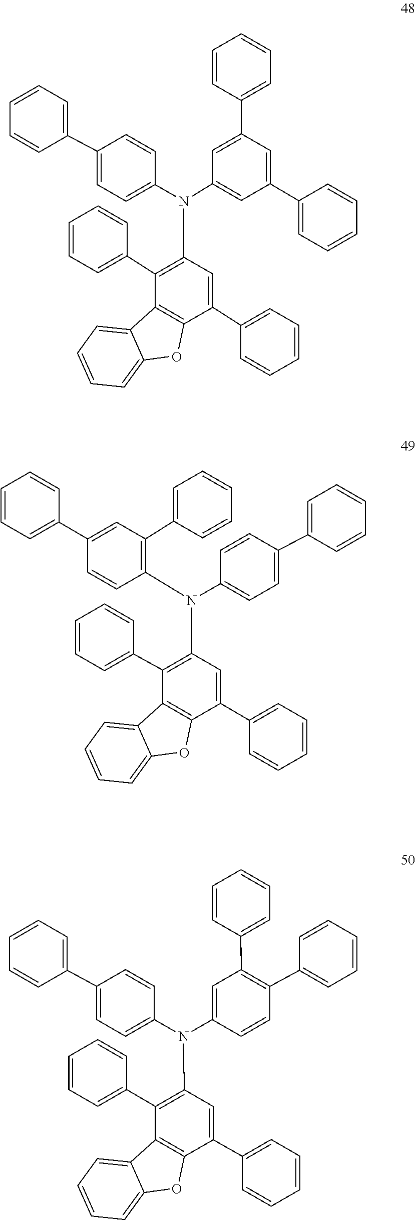 [SMILES: c1ccc(-c2ccc(N(c3cc(-c4ccccc4)cc(-c4ccccc4)c3)c3cc(-c4ccccc4)c4oc5ccccc5c4c3-c3ccccc3)cc2)cc1 ; c1ccc(-c2ccc(N(c3ccc(-c4ccccc4)cc3-c3ccccc3)c3cc(-c4ccccc4)c4oc5ccccc5c4c3-c3ccccc3)cc2)cc1 ; c1ccc(-c2ccc(N(c3ccc(-c4ccccc4)c(-c4ccccc4)c3)c3cc(-c4ccccc4)c4oc5ccccc5c4c3-c3ccccc3)cc2)cc1]

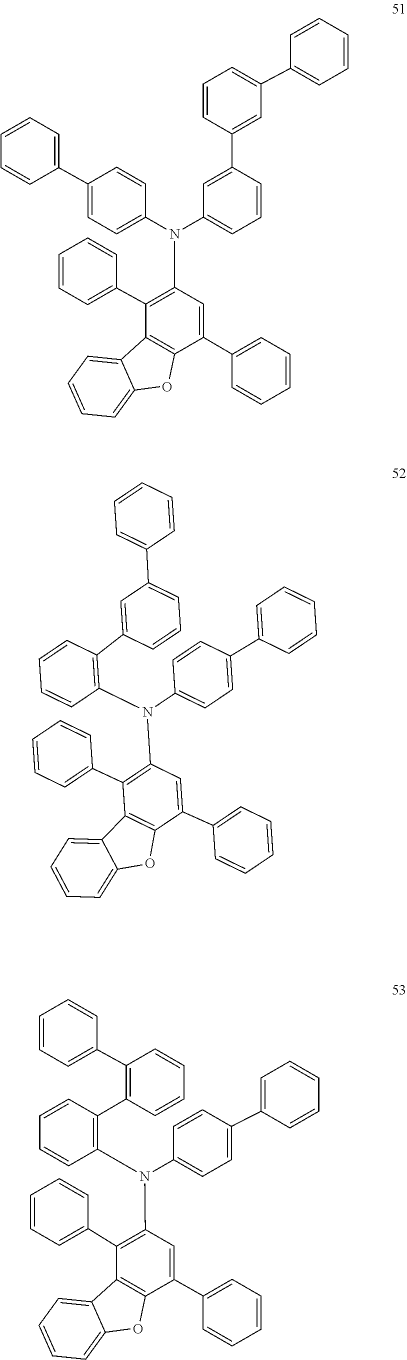 [SMILES: c1ccc(-c2ccc(N(c3cccc(-c4cccc(-c5ccccc5)c4)c3)c3cc(-c4ccccc4)c4oc5ccccc5c4c3-c3ccccc3)cc2)cc1 ; c1ccc(-c2ccc(N(c3ccccc3-c3cccc(-c4ccccc4)c3)c3cc(-c4ccccc4)c4oc5ccccc5c4c3-c3ccccc3)cc2)cc1 ; c1ccc(-c2ccc(N(c3ccccc3-c3ccccc3-c3ccccc3)c3cc(-c4ccccc4)c4oc5ccccc5c4c3-c3ccccc3)cc2)cc1]

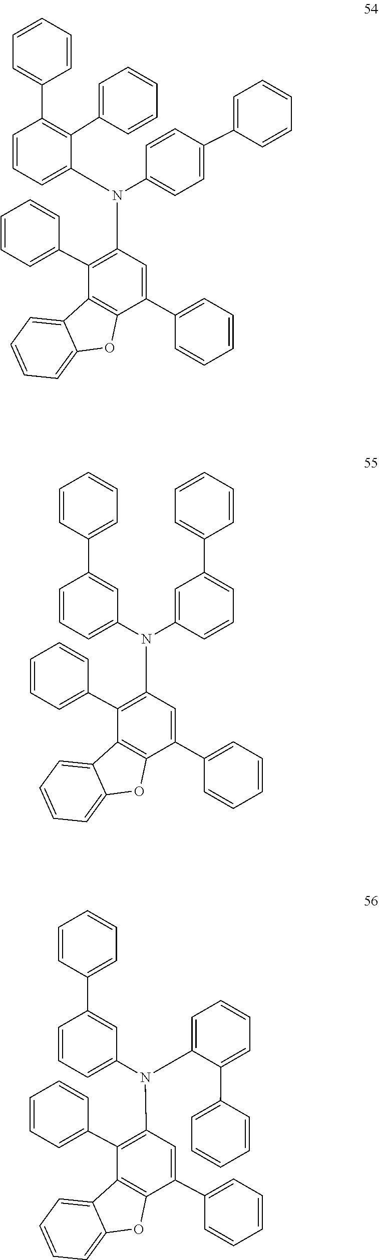 [SMILES: c1ccc(-c2ccc(N(c3cccc(-c4ccccc4)c3-c3ccccc3)c3cc(-c4ccccc4)c4oc5ccccc5c4c3-c3ccccc3)cc2)cc1 ; c1ccc(-c2cccc(N(c3cccc(-c4ccccc4)c3)c3cc(-c4ccccc4)c4oc5ccccc5c4c3-c3ccccc3)c2)cc1 ; c1ccc(-c2cccc(N(c3ccccc3-c3ccccc3)c3cc(-c4ccccc4)c4oc5ccccc5c4c3-c3ccccc3)c2)cc1]

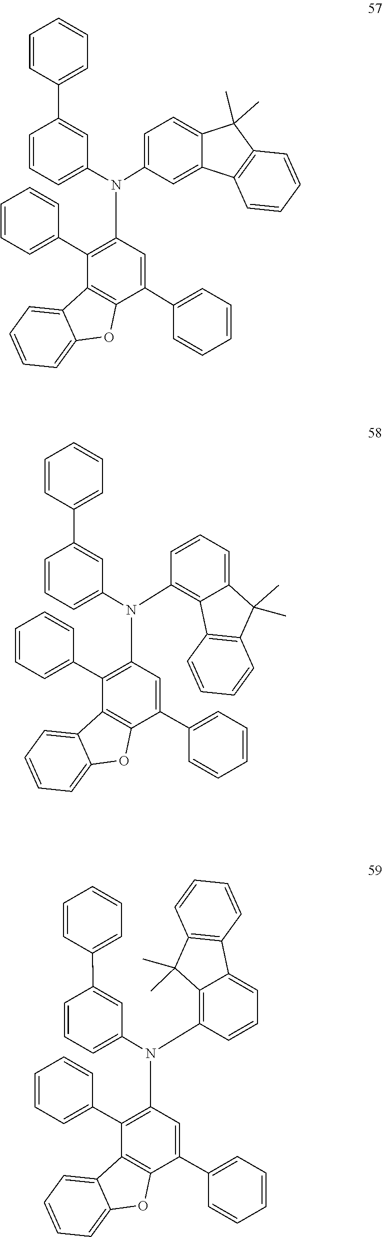 [SMILES: CC1(C)c2ccccc2-c2cc(N(c3cccc(-c4ccccc4)c3)c3cc(-c4ccccc4)c4oc5ccccc5c4c3-c3ccccc3)ccc21 ; CC1(C)c2ccccc2-c2c(N(c3cccc(-c4ccccc4)c3)c3cc(-c4ccccc4)c4oc5ccccc5c4c3-c3ccccc3)cccc21 ; CC1(C)c2ccccc2-c2cccc(N(c3cccc(-c4ccccc4)c3)c3cc(-c4ccccc4)c4oc5ccccc5c4c3-c3ccccc3)c21]

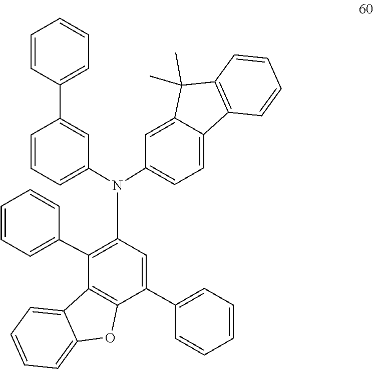 [SMILES: CC1(C)c2ccccc2-c2ccc(N(c3cccc(-c4ccccc4)c3)c3cc(-c4ccccc4)c4oc5ccccc5c4c3-c3ccccc3)cc21]

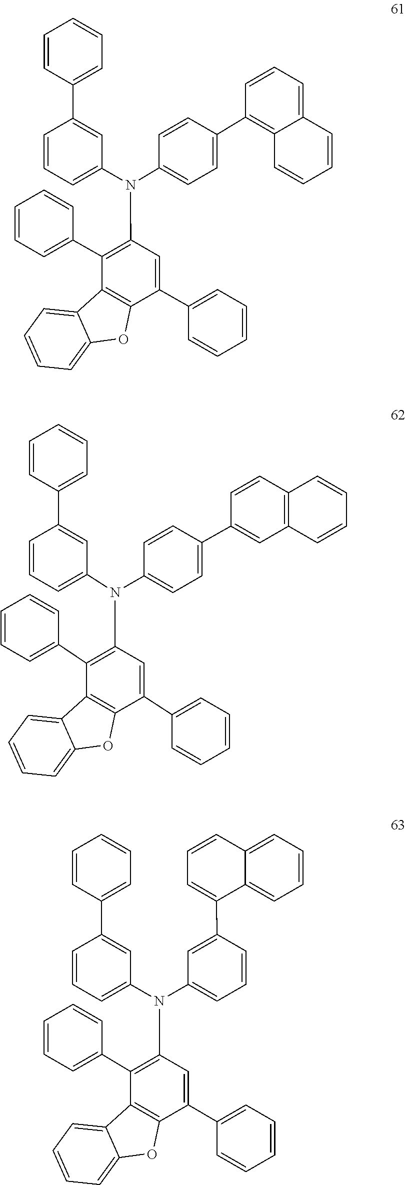 [SMILES: c1ccc(-c2cccc(N(c3ccc(-c4cccc5ccccc45)cc3)c3cc(-c4ccccc4)c4oc5ccccc5c4c3-c3ccccc3)c2)cc1 ; c1ccc(-c2cccc(N(c3ccc(-c4ccc5ccccc5c4)cc3)c3cc(-c4ccccc4)c4oc5ccccc5c4c3-c3ccccc3)c2)cc1 ; c1ccc(-c2cccc(N(c3cccc(-c4cccc5ccccc45)c3)c3cc(-c4ccccc4)c4oc5ccccc5c4c3-c3ccccc3)c2)cc1]

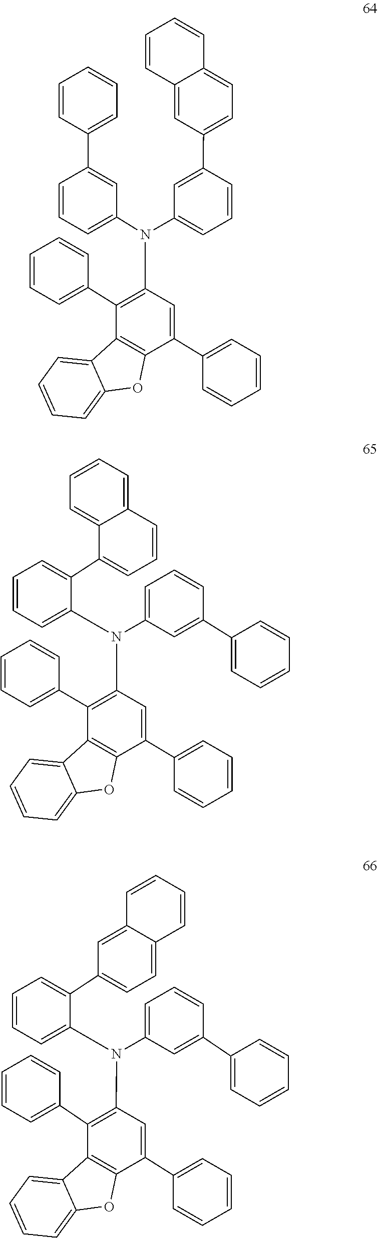

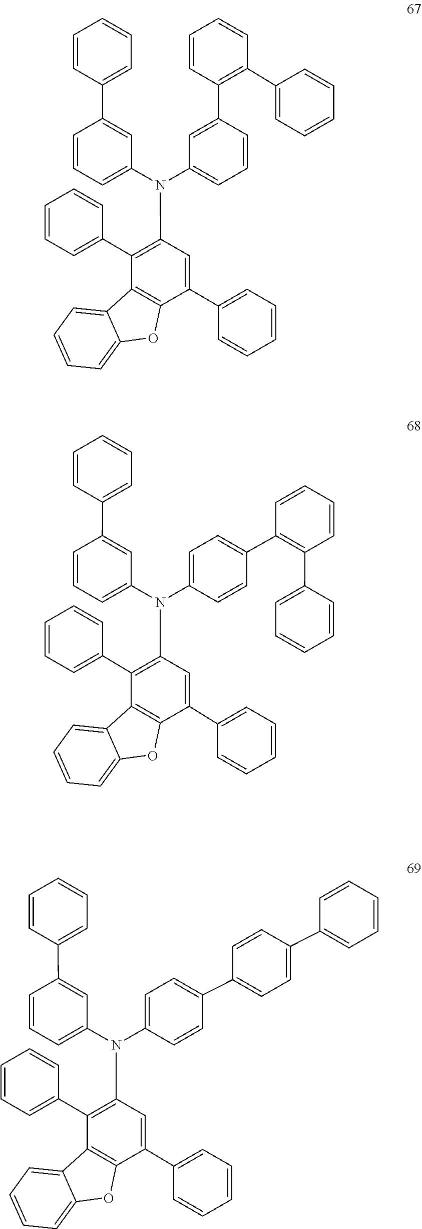 [SMILES: c1ccc(-c2cccc(N(c3cccc(-c4ccccc4-c4ccccc4)c3)c3cc(-c4ccccc4)c4oc5ccccc5c4c3-c3ccccc3)c2)cc1 ; c1ccc(-c2cccc(N(c3ccc(-c4ccccc4-c4ccccc4)cc3)c3cc(-c4ccccc4)c4oc5ccccc5c4c3-c3ccccc3)c2)cc1 ; c1ccc(-c2ccc(-c3ccc(N(c4cccc(-c5ccccc5)c4)c4cc(-c5ccccc5)c5oc6ccccc6c5c4-c4ccccc4)cc3)cc2)cc1]

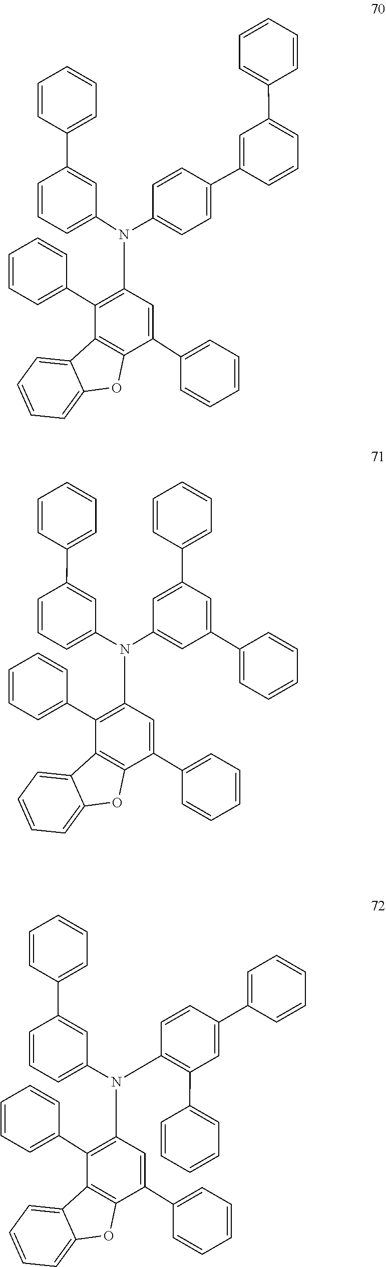 [SMILES: c1ccc(-c2cccc(-c3ccc(N(c4cccc(-c5ccccc5)c4)c4cc(-c5ccccc5)c5oc6ccccc6c5c4-c4ccccc4)cc3)c2)cc1 ; c1ccc(-c2cccc(N(c3cc(-c4ccccc4)cc(-c4ccccc4)c3)c3cc(-c4ccccc4)c4oc5ccccc5c4c3-c3ccccc3)c2)cc1 ; c1ccc(-c2cccc(N(c3ccc(-c4ccccc4)cc3-c3ccccc3)c3cc(-c4ccccc4)c4oc5ccccc5c4c3-c3ccccc3)c2)cc1]

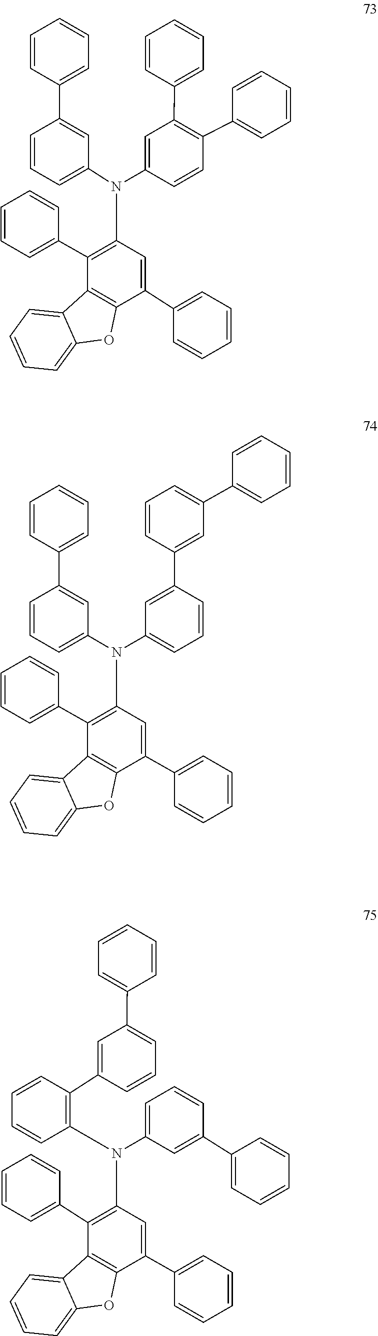 [SMILES: c1ccc(-c2cccc(N(c3ccc(-c4ccccc4)c(-c4ccccc4)c3)c3cc(-c4ccccc4)c4oc5ccccc5c4c3-c3ccccc3)c2)cc1 ; c1ccc(-c2cccc(-c3cccc(N(c4cccc(-c5ccccc5)c4)c4cc(-c5ccccc5)c5oc6ccccc6c5c4-c4ccccc4)c3)c2)cc1 ; c1ccc(-c2cccc(-c3ccccc3N(c3cccc(-c4ccccc4)c3)c3cc(-c4ccccc4)c4oc5ccccc5c4c3-c3ccccc3)c2)cc1]

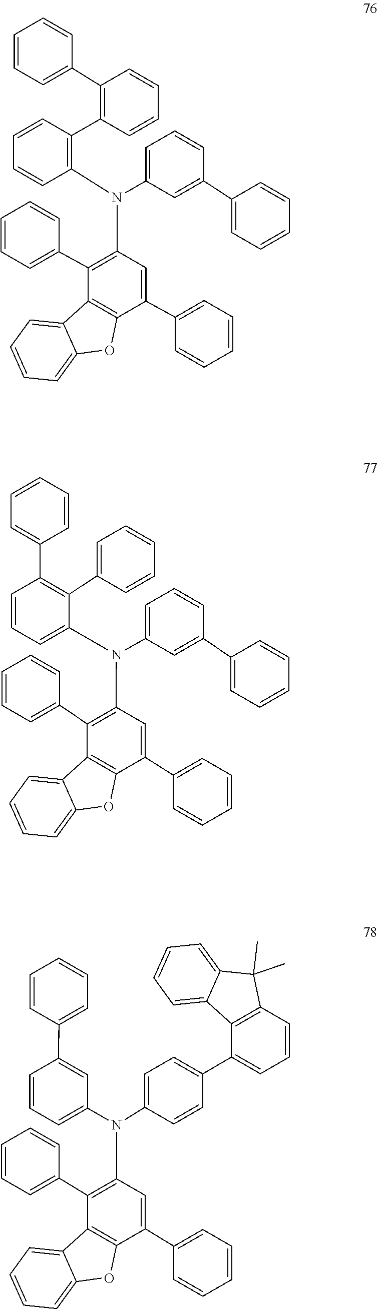 [SMILES: c1ccc(-c2cccc(N(c3ccccc3-c3ccccc3-c3ccccc3)c3cc(-c4ccccc4)c4oc5ccccc5c4c3-c3ccccc3)c2)cc1 ; c1ccc(-c2cccc(N(c3cccc(-c4ccccc4)c3-c3ccccc3)c3cc(-c4ccccc4)c4oc5ccccc5c4c3-c3ccccc3)c2)cc1 ; CC1(C)c2ccccc2-c2c(-c3ccc(N(c4cccc(-c5ccccc5)c4)c4cc(-c5ccccc5)c5oc6ccccc6c5c4-c4ccccc4)cc3)cccc21]

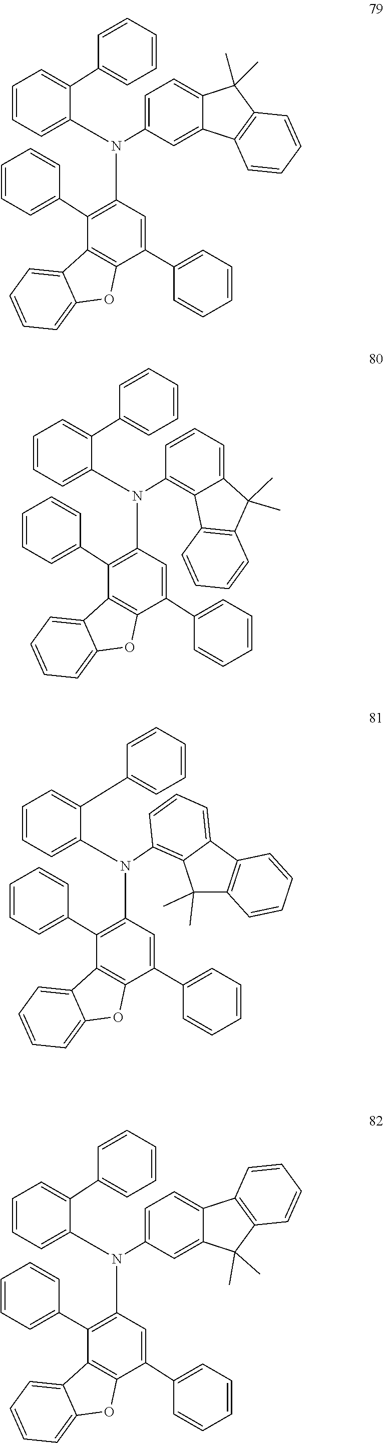 [SMILES: CC1(C)c2ccccc2-c2cc(N(c3ccccc3-c3ccccc3)c3cc(-c4ccccc4)c4oc5ccccc5c4c3-c3ccccc3)ccc21 ; CC1(C)c2ccccc2-c2c(N(c3ccccc3-c3ccccc3)c3cc(-c4ccccc4)c4oc5ccccc5c4c3-c3ccccc3)cccc21 ; CC1(C)c2ccccc2-c2cccc(N(c3ccccc3-c3ccccc3)c3cc(-c4ccccc4)c4oc5ccccc5c4c3-c3ccccc3)c21 ; CC1(C)c2ccccc2-c2ccc(N(c3ccccc3-c3ccccc3)c3cc(-c4ccccc4)c4oc5ccccc5c4c3-c3ccccc3)cc21]

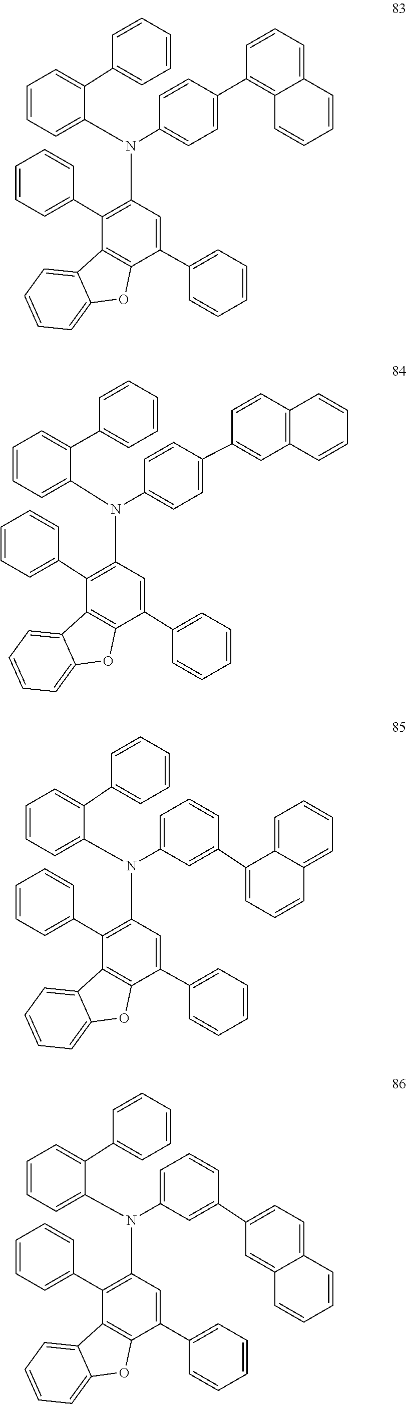 [SMILES: c1ccc(-c2ccccc2N(c2ccc(-c3cccc4ccccc34)cc2)c2cc(-c3ccccc3)c3oc4ccccc4c3c2-c2ccccc2)cc1 ; c1ccc(-c2ccccc2N(c2ccc(-c3ccc4ccccc4c3)cc2)c2cc(-c3ccccc3)c3oc4ccccc4c3c2-c2ccccc2)cc1 ; c1ccc(-c2ccccc2N(c2cccc(-c3cccc4ccccc34)c2)c2cc(-c3ccccc3)c3oc4ccccc4c3c2-c2ccccc2)cc1 ; c1ccc(-c2ccccc2N(c2cccc(-c3ccc4ccccc4c3)c2)c2cc(-c3ccccc3)c3oc4ccccc4c3c2-c2ccccc2)cc1]

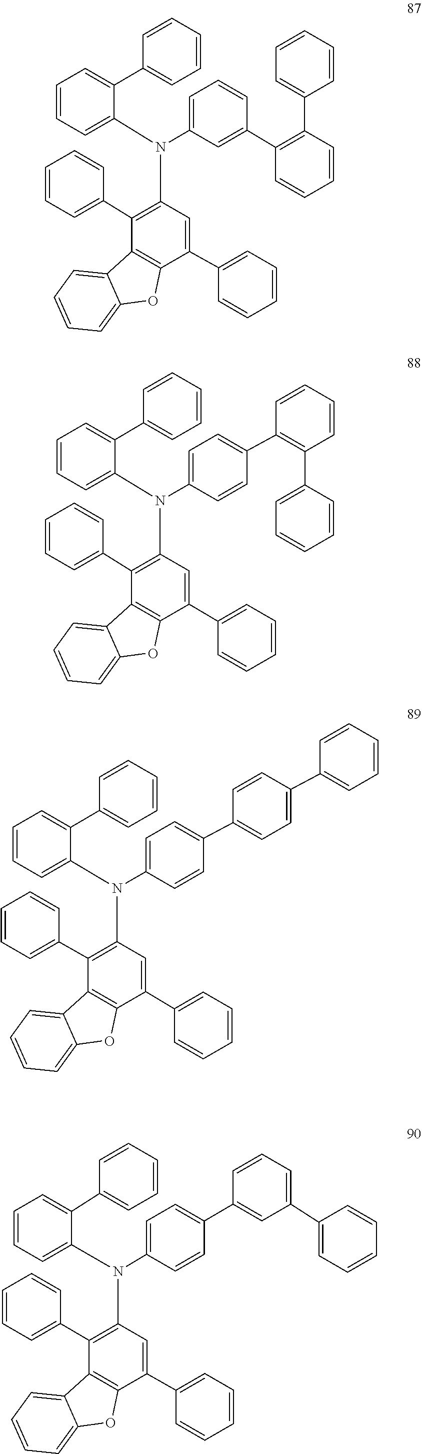 [SMILES: c1ccc(-c2ccccc2-c2cccc(N(c3ccccc3-c3ccccc3)c3cc(-c4ccccc4)c4oc5ccccc5c4c3-c3ccccc3)c2)cc1 ; c1ccc(-c2ccccc2-c2ccc(N(c3ccccc3-c3ccccc3)c3cc(-c4ccccc4)c4oc5ccccc5c4c3-c3ccccc3)cc2)cc1 ; c1ccc(-c2ccc(-c3ccc(N(c4ccccc4-c4ccccc4)c4cc(-c5ccccc5)c5oc6ccccc6c5c4-c4ccccc4)cc3)cc2)cc1 ; c1ccc(-c2cccc(-c3ccc(N(c4ccccc4-c4ccccc4)c4cc(-c5ccccc5)c5oc6ccccc6c5c4-c4ccccc4)cc3)c2)cc1]

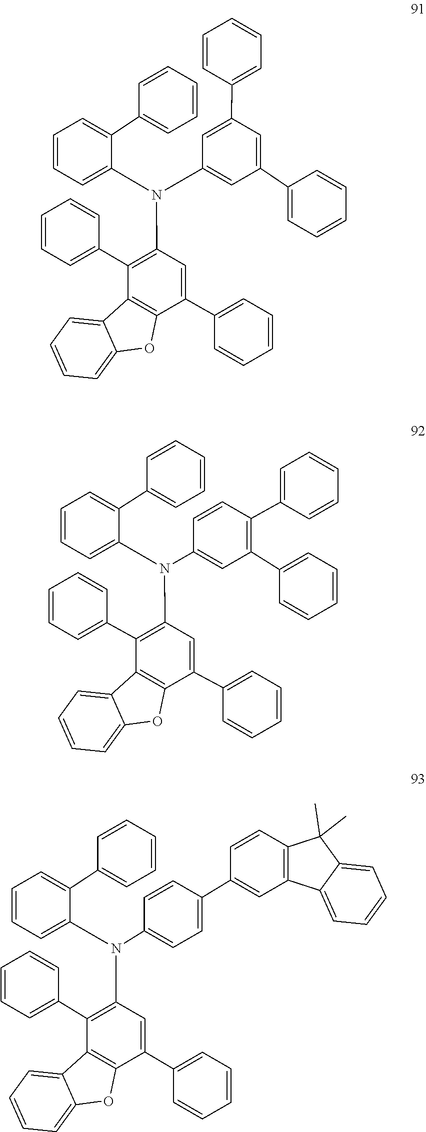 [SMILES: c1ccc(-c2cc(-c3ccccc3)cc(N(c3ccccc3-c3ccccc3)c3cc(-c4ccccc4)c4oc5ccccc5c4c3-c3ccccc3)c2)cc1 ; c1ccc(-c2ccc(N(c3ccccc3-c3ccccc3)c3cc(-c4ccccc4)c4oc5ccccc5c4c3-c3ccccc3)cc2-c2ccccc2)cc1 ; CC1(C)c2ccccc2-c2cc(-c3ccc(N(c4ccccc4-c4ccccc4)c4cc(-c5ccccc5)c5oc6ccccc6c5c4-c4ccccc4)cc3)ccc21]

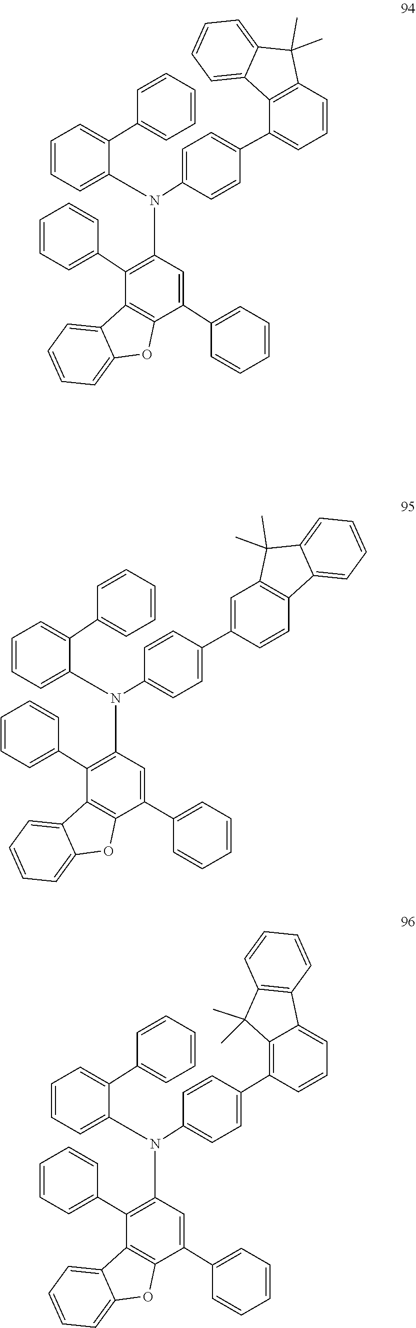 [SMILES: CC1(C)c2ccccc2-c2c(-c3ccc(N(c4ccccc4-c4ccccc4)c4cc(-c5ccccc5)c5oc6ccccc6c5c4-c4ccccc4)cc3)cccc21 ; CC1(C)c2ccccc2-c2ccc(-c3ccc(N(c4ccccc4-c4ccccc4)c4cc(-c5ccccc5)c5oc6ccccc6c5c4-c4ccccc4)cc3)cc21 ; CC1(C)c2ccccc2-c2cccc(-c3ccc(N(c4ccccc4-c4ccccc4)c4cc(-c5ccccc5)c5oc6ccccc6c5c4-c4ccccc4)cc3)c21]

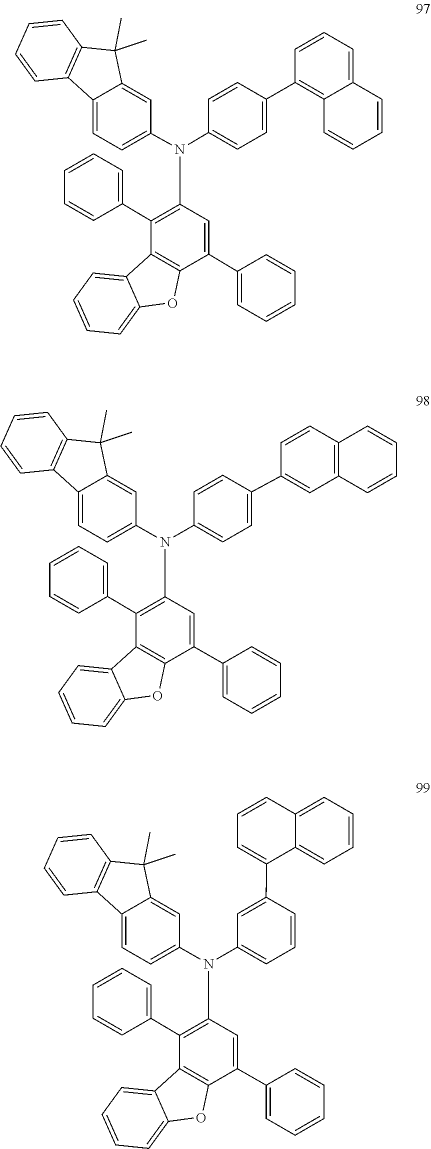 [SMILES: CC1(C)c2ccccc2-c2ccc(N(c3ccc(-c4cccc5ccccc45)cc3)c3cc(-c4ccccc4)c4oc5ccccc5c4c3-c3ccccc3)cc21 ; CC1(C)c2ccccc2-c2ccc(N(c3ccc(-c4ccc5ccccc5c4)cc3)c3cc(-c4ccccc4)c4oc5ccccc5c4c3-c3ccccc3)cc21 ; CC1(C)c2ccccc2-c2ccc(N(c3cccc(-c4cccc5ccccc45)c3)c3cc(-c4ccccc4)c4oc5ccccc5c4c3-c3ccccc3)cc21]

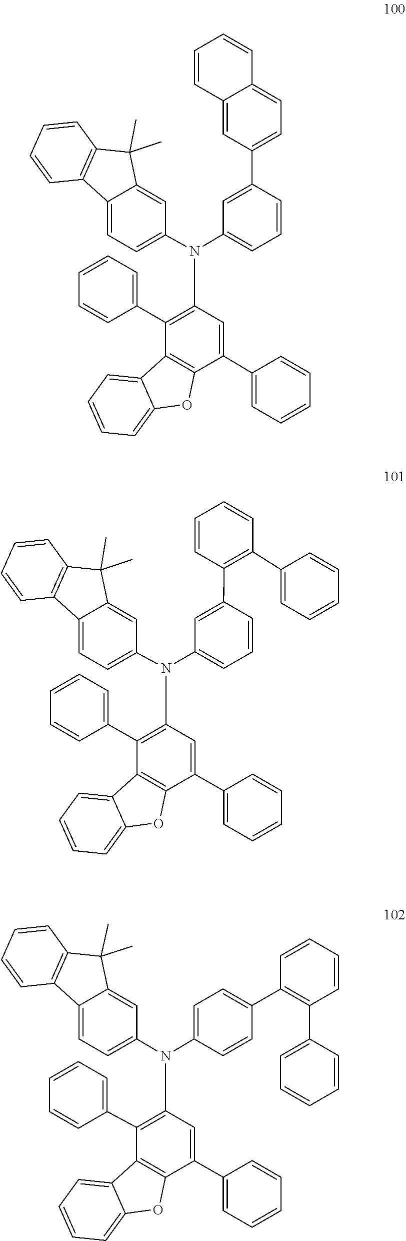 [SMILES: CC1(C)c2ccccc2-c2ccc(N(c3cccc(-c4ccc5ccccc5c4)c3)c3cc(-c4ccccc4)c4oc5ccccc5c4c3-c3ccccc3)cc21 ; CC1(C)c2ccccc2-c2ccc(N(c3cccc(-c4ccccc4-c4ccccc4)c3)c3cc(-c4ccccc4)c4oc5ccccc5c4c3-c3ccccc3)cc21 ; CC1(C)c2ccccc2-c2ccc(N(c3ccc(-c4ccccc4-c4ccccc4)cc3)c3cc(-c4ccccc4)c4oc5ccccc5c4c3-c3ccccc3)cc21]

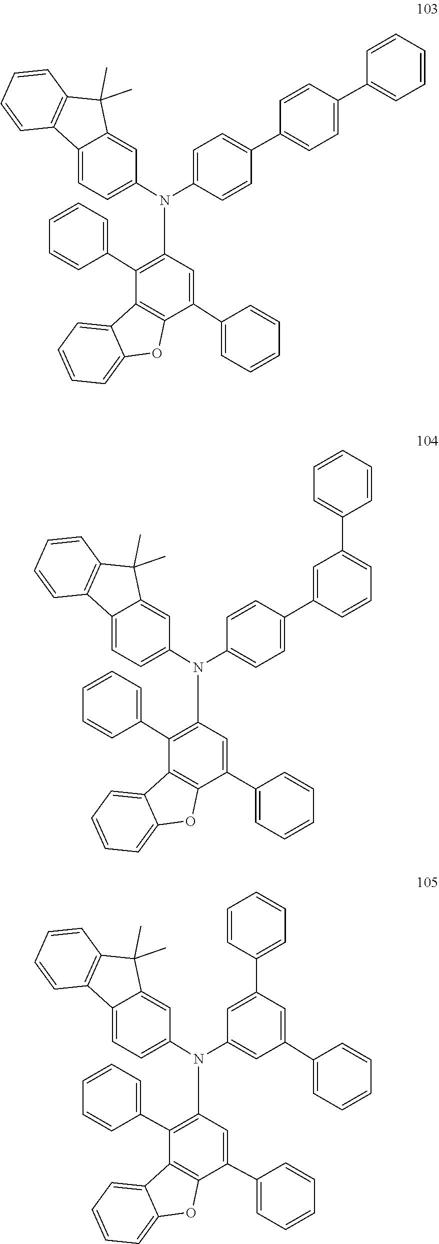 [SMILES: CC1(C)c2ccccc2-c2ccc(N(c3ccc(-c4ccc(-c5ccccc5)cc4)cc3)c3cc(-c4ccccc4)c4oc5ccccc5c4c3-c3ccccc3)cc21 ; CC1(C)c2ccccc2-c2ccc(N(c3ccc(-c4cccc(-c5ccccc5)c4)cc3)c3cc(-c4ccccc4)c4oc5ccccc5c4c3-c3ccccc3)cc21 ; CC1(C)c2ccccc2-c2ccc(N(c3cc(-c4ccccc4)cc(-c4ccccc4)c3)c3cc(-c4ccccc4)c4oc5ccccc5c4c3-c3ccccc3)cc21]

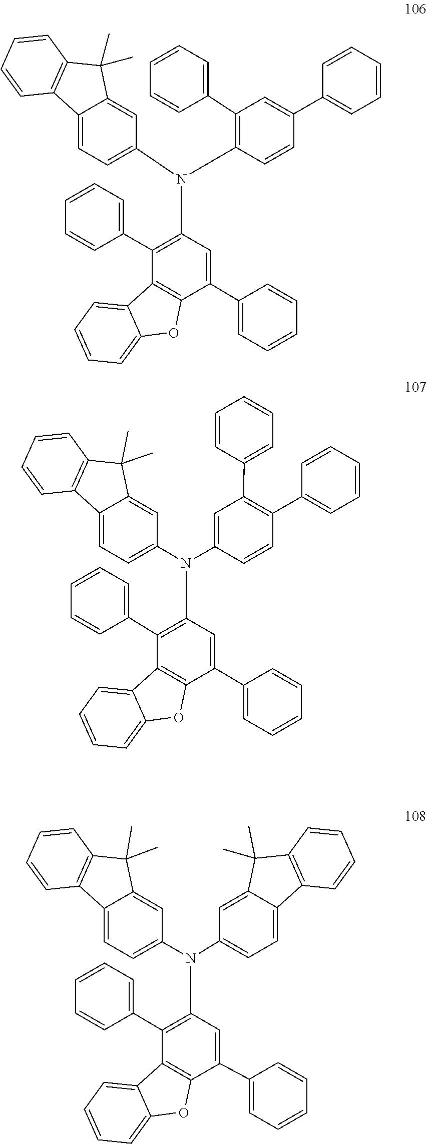 [SMILES: CC1(C)c2ccccc2-c2ccc(N(c3ccc(-c4ccccc4)cc3-c3ccccc3)c3cc(-c4ccccc4)c4oc5ccccc5c4c3-c3ccccc3)cc21 ; CC1(C)c2ccccc2-c2ccc(N(c3ccc(-c4ccccc4)c(-c4ccccc4)c3)c3cc(-c4ccccc4)c4oc5ccccc5c4c3-c3ccccc3)cc21 ; CC1(C)c2ccccc2-c2ccc(N(c3ccc4c(c3)C(C)(C)c3ccccc3-4)c3cc(-c4ccccc4)c4oc5ccccc5c4c3-c3ccccc3)cc21]

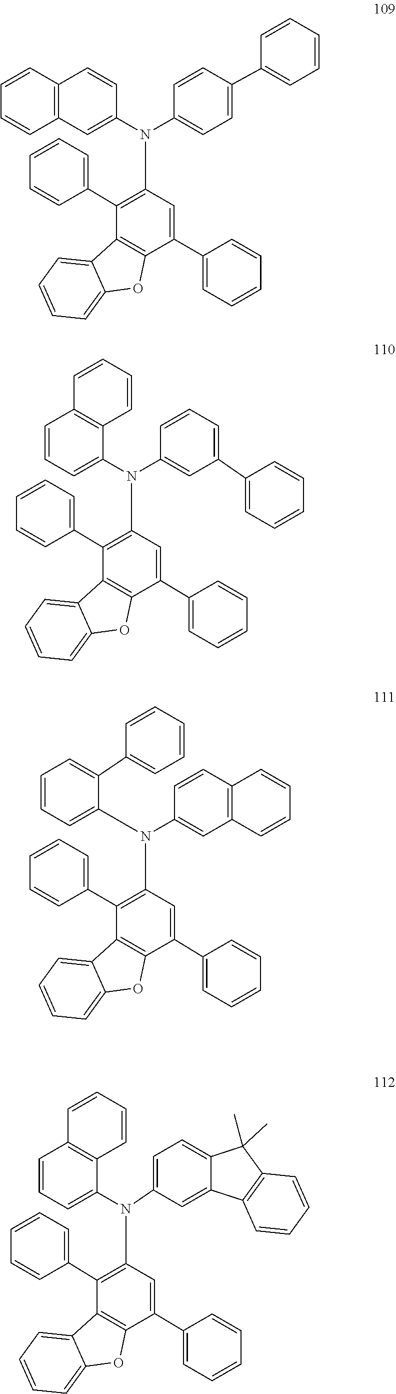

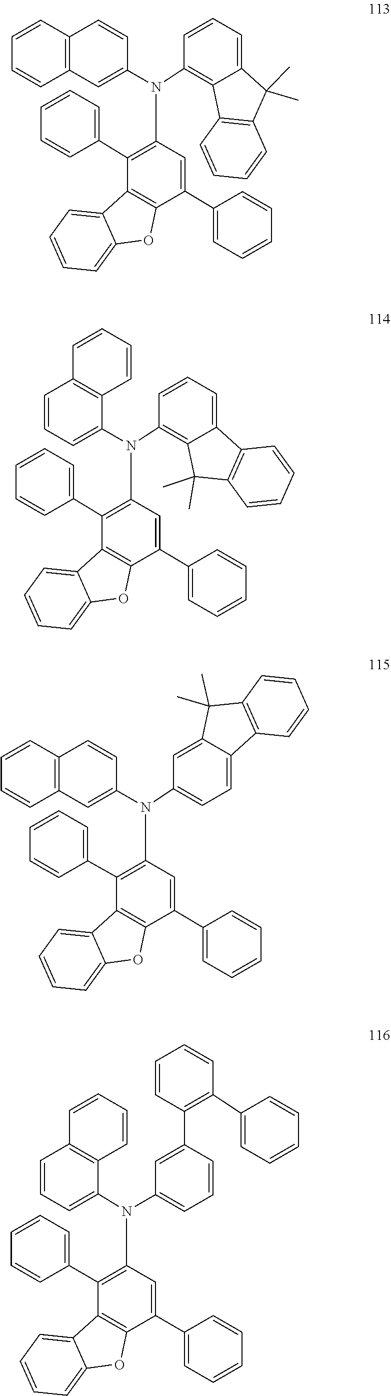 [SMILES: CC1(C)c2ccccc2-c2c(N(c3ccc4ccccc4c3)c3cc(-c4ccccc4)c4oc5ccccc5c4c3-c3ccccc3)cccc21 ; CC1(C)c2ccccc2-c2cccc(N(c3cc(-c4ccccc4)c4oc5ccccc5c4c3-c3ccccc3)c3cccc4ccccc34)c21 ; CC1(C)c2ccccc2-c2ccc(N(c3ccc4ccccc4c3)c3cc(-c4ccccc4)c4oc5ccccc5c4c3-c3ccccc3)cc21 ; c1ccc(-c2ccccc2-c2cccc(N(c3cc(-c4ccccc4)c4oc5ccccc5c4c3-c3ccccc3)c3cccc4ccccc34)c2)cc1]

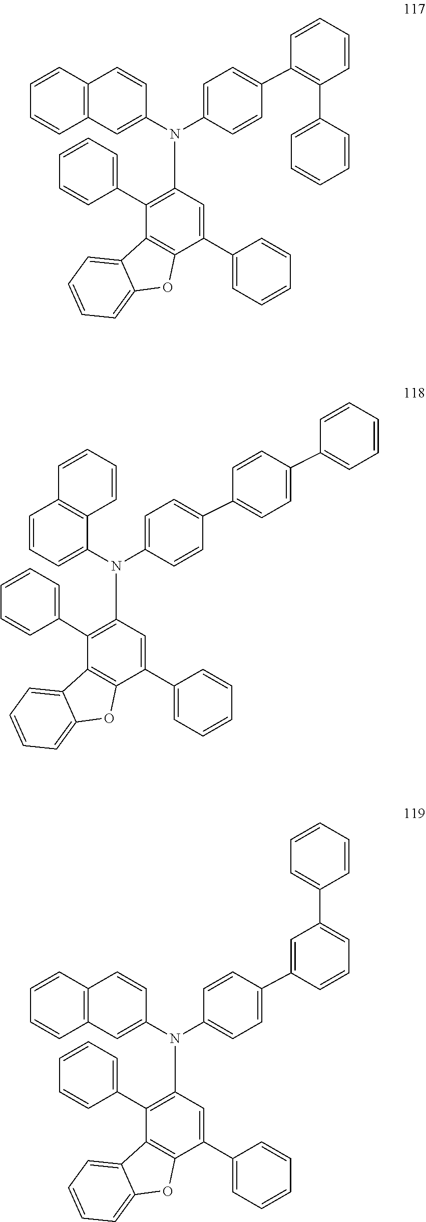 [SMILES: c1ccc(-c2ccccc2-c2ccc(N(c3ccc4ccccc4c3)c3cc(-c4ccccc4)c4oc5ccccc5c4c3-c3ccccc3)cc2)cc1 ; c1ccc(-c2ccc(-c3ccc(N(c4cc(-c5ccccc5)c5oc6ccccc6c5c4-c4ccccc4)c4cccc5ccccc45)cc3)cc2)cc1 ; c1ccc(-c2cccc(-c3ccc(N(c4ccc5ccccc5c4)c4cc(-c5ccccc5)c5oc6ccccc6c5c4-c4ccccc4)cc3)c2)cc1]

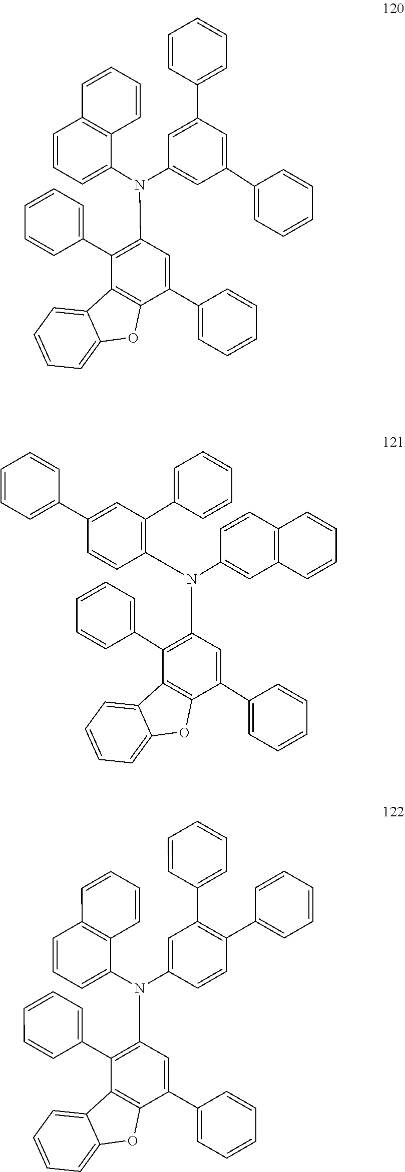 [SMILES: c1ccc(-c2cc(-c3ccccc3)cc(N(c3cc(-c4ccccc4)c4oc5ccccc5c4c3-c3ccccc3)c3cccc4ccccc34)c2)cc1 ; c1ccc(-c2ccc(N(c3ccc4ccccc4c3)c3cc(-c4ccccc4)c4oc5ccccc5c4c3-c3ccccc3)c(-c3ccccc3)c2)cc1 ; c1ccc(-c2ccc(N(c3cc(-c4ccccc4)c4oc5ccccc5c4c3-c3ccccc3)c3cccc4ccccc34)cc2-c2ccccc2)cc1]

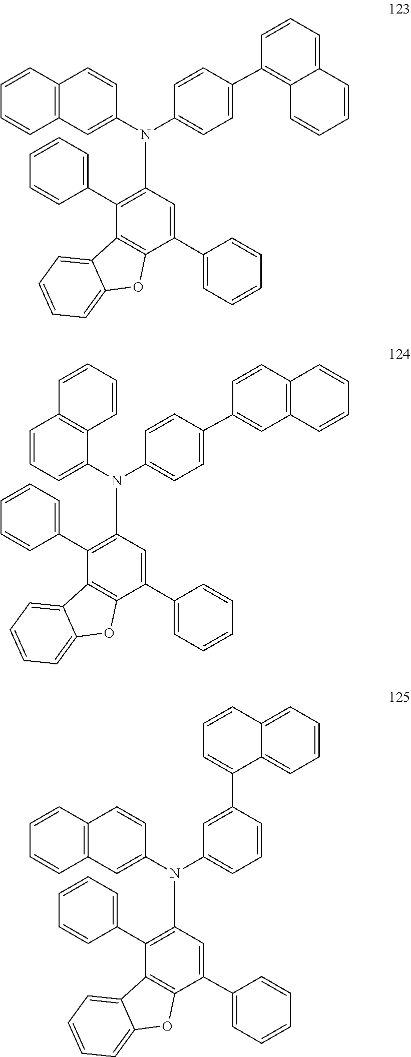

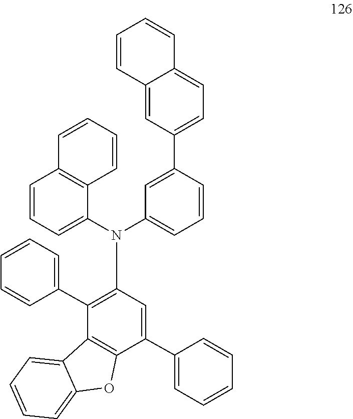 [SMILES: c1ccc(-c2cc(N(c3cccc(-c4ccc5ccccc5c4)c3)c3cccc4ccccc34)c(-c3ccccc3)c3c2oc2ccccc23)cc1]

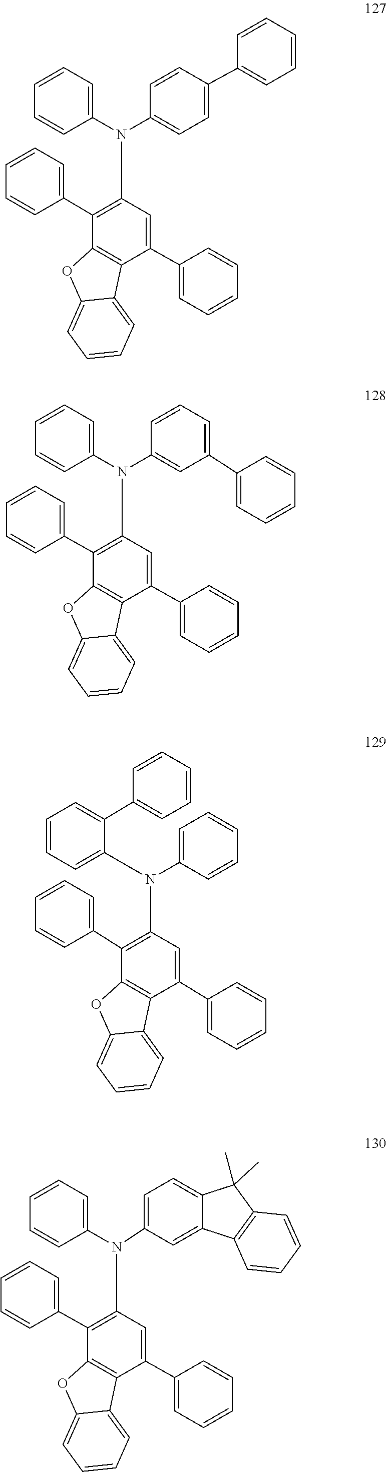 [SMILES: c1ccc(-c2ccc(N(c3ccccc3)c3cc(-c4ccccc4)c4c(oc5ccccc54)c3-c3ccccc3)cc2)cc1 ; c1ccc(-c2cccc(N(c3ccccc3)c3cc(-c4ccccc4)c4c(oc5ccccc54)c3-c3ccccc3)c2)cc1 ; c1ccc(-c2ccccc2N(c2ccccc2)c2cc(-c3ccccc3)c3c(oc4ccccc43)c2-c2ccccc2)cc1 ; CC1(C)c2ccccc2-c2cc(N(c3ccccc3)c3cc(-c4ccccc4)c4c(oc5ccccc54)c3-c3ccccc3)ccc21]

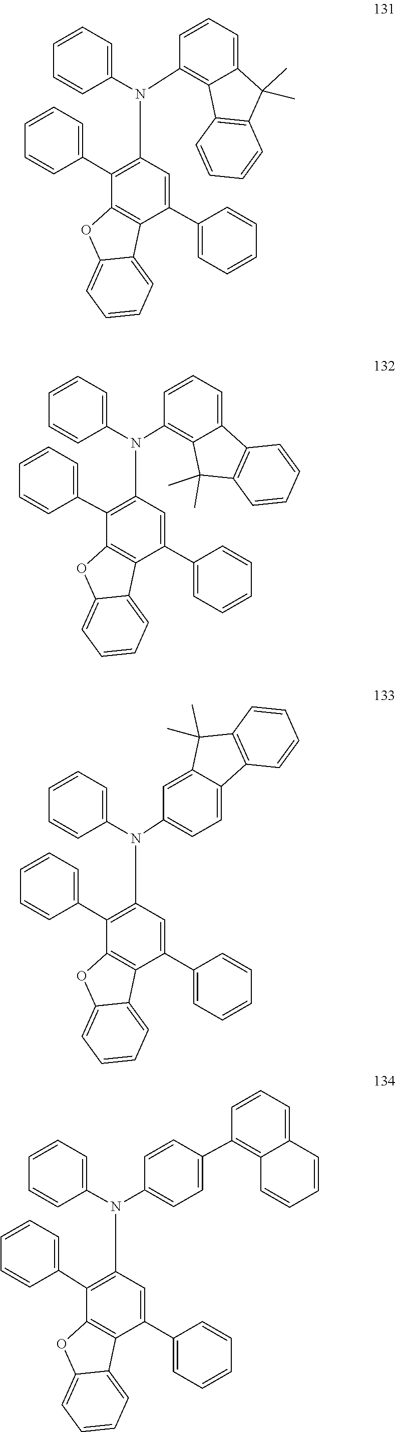 [SMILES: CC1(C)c2ccccc2-c2c(N(c3ccccc3)c3cc(-c4ccccc4)c4c(oc5ccccc54)c3-c3ccccc3)cccc21 ; CC1(C)c2ccccc2-c2cccc(N(c3ccccc3)c3cc(-c4ccccc4)c4c(oc5ccccc54)c3-c3ccccc3)c21 ; CC1(C)c2ccccc2-c2ccc(N(c3ccccc3)c3cc(-c4ccccc4)c4c(oc5ccccc54)c3-c3ccccc3)cc21 ; c1ccc(-c2c(N(c3ccccc3)c3ccc(-c4cccc5ccccc45)cc3)cc(-c3ccccc3)c3c2oc2ccccc23)cc1]

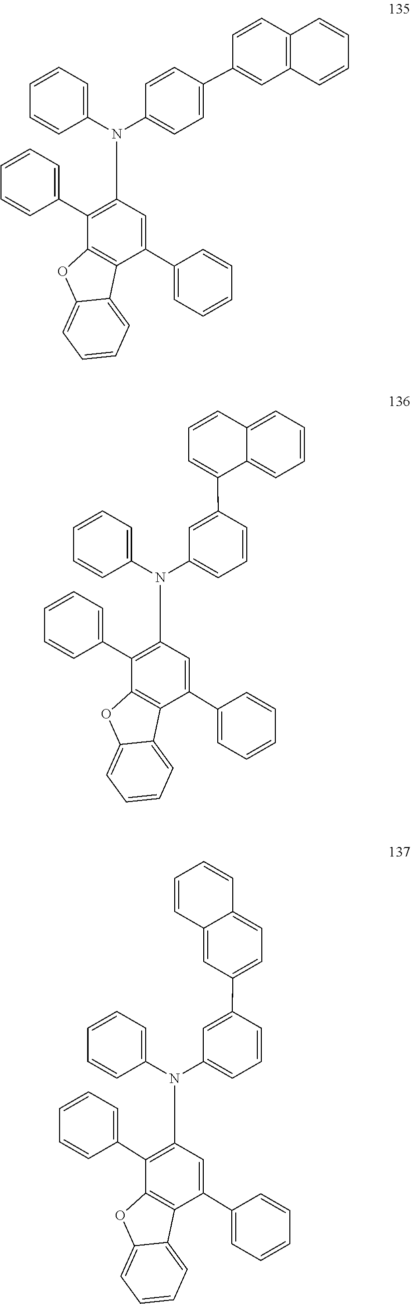 [SMILES: c1ccc(-c2c(N(c3ccccc3)c3ccc(-c4ccc5ccccc5c4)cc3)cc(-c3ccccc3)c3c2oc2ccccc23)cc1 ; c1ccc(-c2c(N(c3ccccc3)c3cccc(-c4cccc5ccccc45)c3)cc(-c3ccccc3)c3c2oc2ccccc23)cc1 ; c1ccc(-c2c(N(c3ccccc3)c3cccc(-c4ccc5ccccc5c4)c3)cc(-c3ccccc3)c3c2oc2ccccc23)cc1]

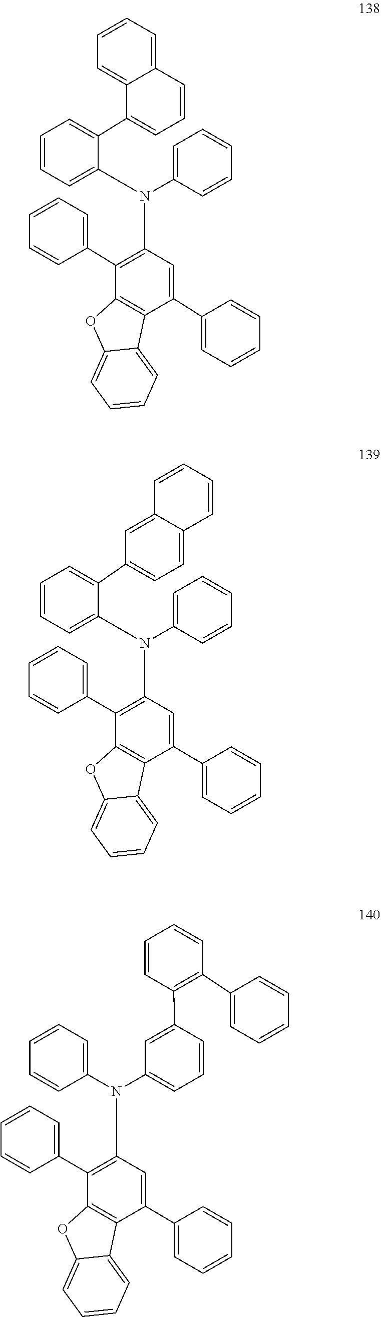 [SMILES: c1ccc(-c2c(N(c3ccccc3)c3ccccc3-c3cccc4ccccc34)cc(-c3ccccc3)c3c2oc2ccccc23)cc1 ; c1ccc(-c2c(N(c3ccccc3)c3ccccc3-c3ccc4ccccc4c3)cc(-c3ccccc3)c3c2oc2ccccc23)cc1 ; c1ccc(-c2ccccc2-c2cccc(N(c3ccccc3)c3cc(-c4ccccc4)c4c(oc5ccccc54)c3-c3ccccc3)c2)cc1]

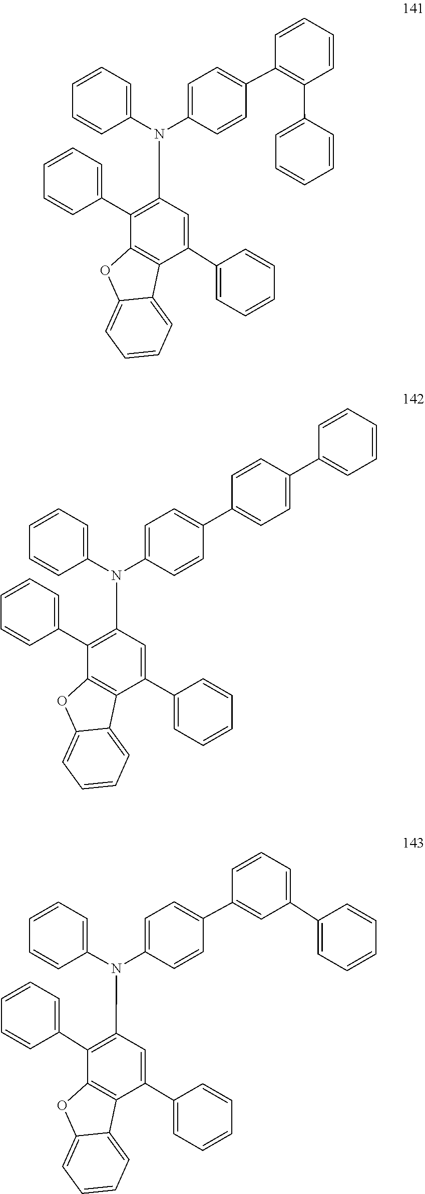 [SMILES: c1ccc(-c2ccccc2-c2ccc(N(c3ccccc3)c3cc(-c4ccccc4)c4c(oc5ccccc54)c3-c3ccccc3)cc2)cc1 ; c1ccc(-c2ccc(-c3ccc(N(c4ccccc4)c4cc(-c5ccccc5)c5c(oc6ccccc65)c4-c4ccccc4)cc3)cc2)cc1 ; c1ccc(-c2cccc(-c3ccc(N(c4ccccc4)c4cc(-c5ccccc5)c5c(oc6ccccc65)c4-c4ccccc4)cc3)c2)cc1]

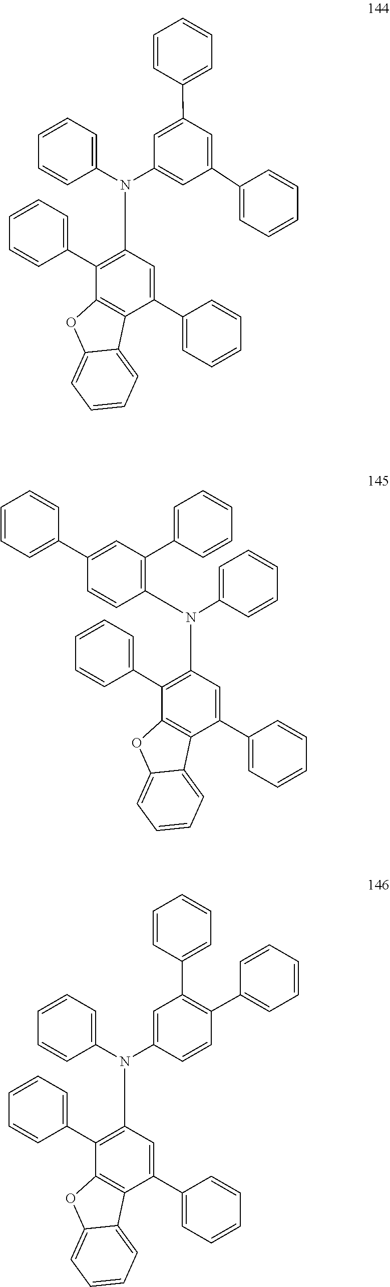 [SMILES: c1ccc(-c2cc(-c3ccccc3)cc(N(c3ccccc3)c3cc(-c4ccccc4)c4c(oc5ccccc54)c3-c3ccccc3)c2)cc1 ; c1ccc(-c2ccc(N(c3ccccc3)c3cc(-c4ccccc4)c4c(oc5ccccc54)c3-c3ccccc3)c(-c3ccccc3)c2)cc1 ; c1ccc(-c2ccc(N(c3ccccc3)c3cc(-c4ccccc4)c4c(oc5ccccc54)c3-c3ccccc3)cc2-c2ccccc2)cc1]

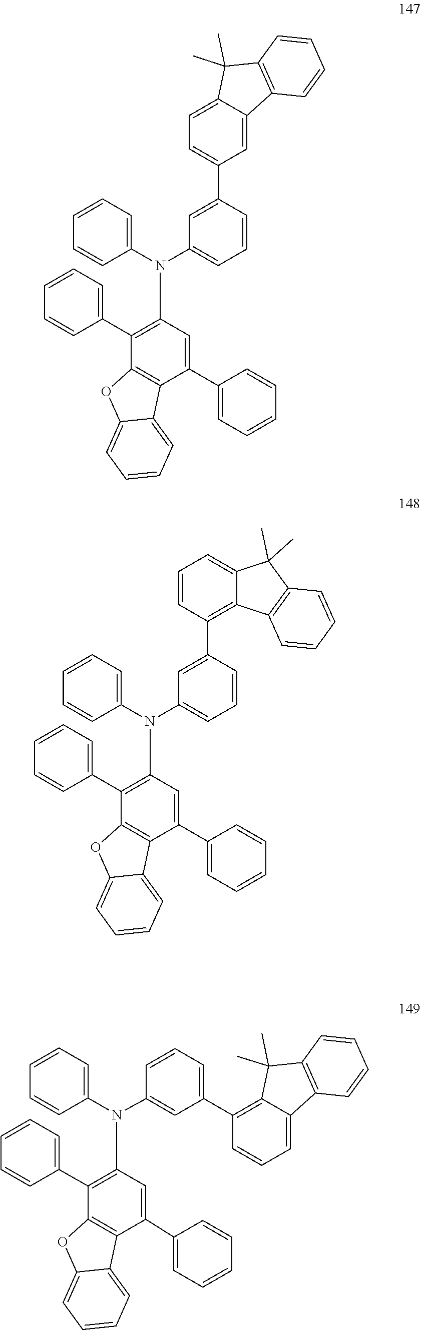 [SMILES: CC1(C)c2ccccc2-c2cc(-c3cccc(N(c4ccccc4)c4cc(-c5ccccc5)c5c(oc6ccccc65)c4-c4ccccc4)c3)ccc21 ; CC1(C)c2ccccc2-c2c(-c3cccc(N(c4ccccc4)c4cc(-c5ccccc5)c5c(oc6ccccc65)c4-c4ccccc4)c3)cccc21 ; CC1(C)c2ccccc2-c2cccc(-c3cccc(N(c4ccccc4)c4cc(-c5ccccc5)c5c(oc6ccccc65)c4-c4ccccc4)c3)c21]

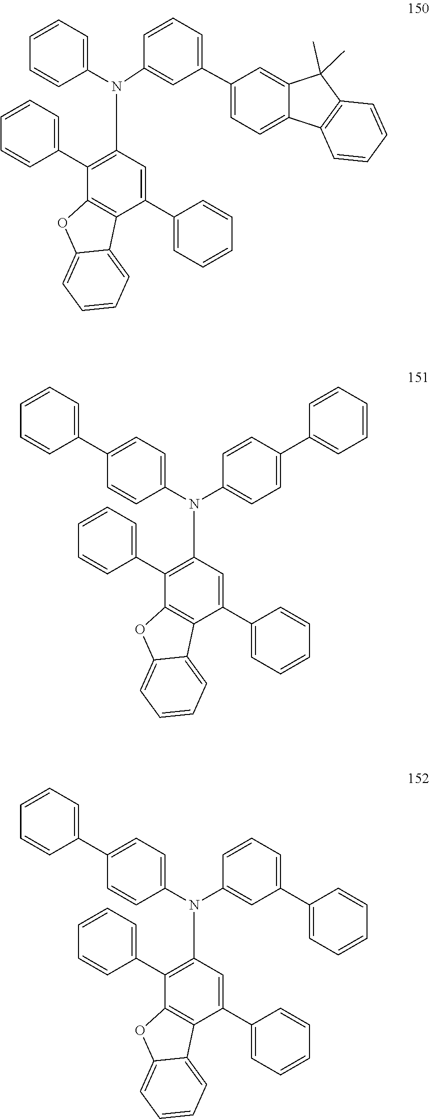 [SMILES: CC1(C)c2ccccc2-c2ccc(-c3cccc(N(c4ccccc4)c4cc(-c5ccccc5)c5c(oc6ccccc65)c4-c4ccccc4)c3)cc21 ; c1ccc(-c2ccc(N(c3ccc(-c4ccccc4)cc3)c3cc(-c4ccccc4)c4c(oc5ccccc54)c3-c3ccccc3)cc2)cc1 ; c1ccc(-c2ccc(N(c3cccc(-c4ccccc4)c3)c3cc(-c4ccccc4)c4c(oc5ccccc54)c3-c3ccccc3)cc2)cc1]

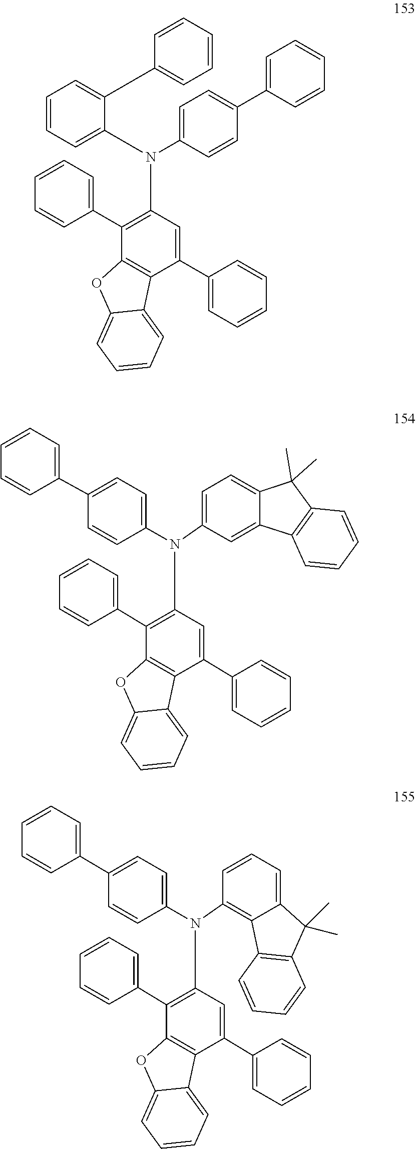 [SMILES: c1ccc(-c2ccc(N(c3ccccc3-c3ccccc3)c3cc(-c4ccccc4)c4c(oc5ccccc54)c3-c3ccccc3)cc2)cc1 ; CC1(C)c2ccccc2-c2cc(N(c3ccc(-c4ccccc4)cc3)c3cc(-c4ccccc4)c4c(oc5ccccc54)c3-c3ccccc3)ccc21 ; CC1(C)c2ccccc2-c2c(N(c3ccc(-c4ccccc4)cc3)c3cc(-c4ccccc4)c4c(oc5ccccc54)c3-c3ccccc3)cccc21]

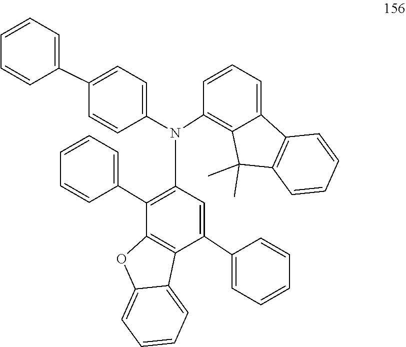 [SMILES: CC1(C)c2ccccc2-c2cccc(N(c3ccc(-c4ccccc4)cc3)c3cc(-c4ccccc4)c4c(oc5ccccc54)c3-c3ccccc3)c21]

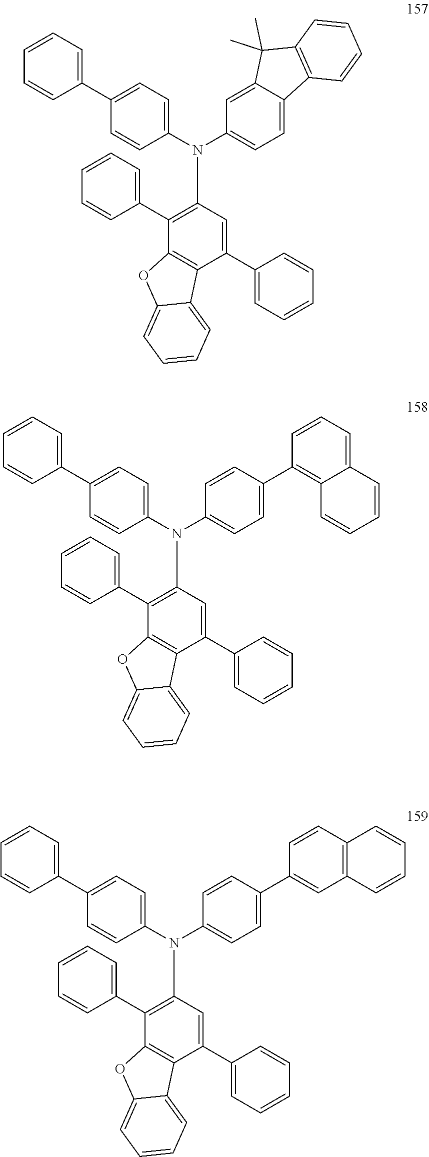

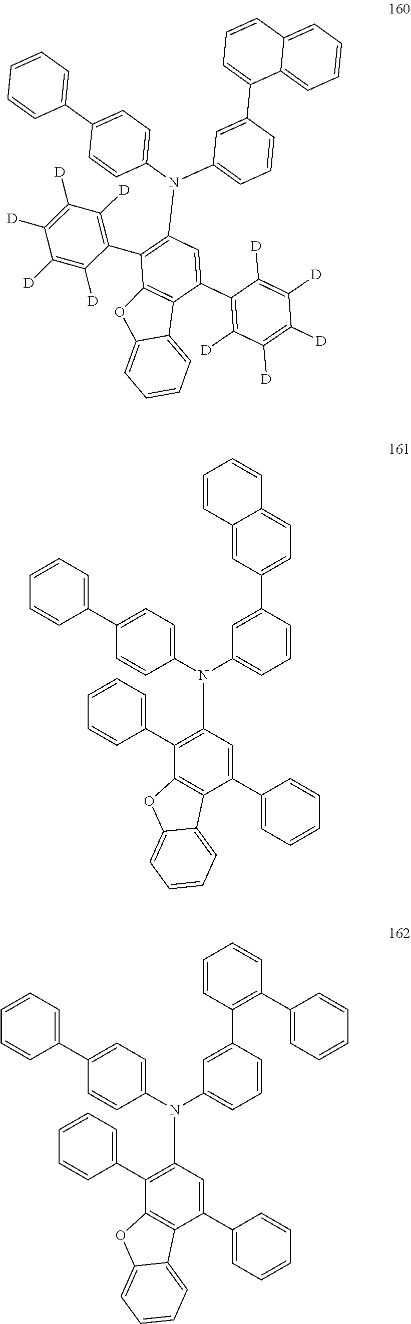 [SMILES: [2H]c1c([2H])c([2H])c(-c2c(N(c3ccc(-c4ccccc4)cc3)c3cccc(-c4cccc5ccccc45)c3)cc(-c3c([2H])c([2H])c([2H])c([2H])c3[2H])c3c2oc2ccccc23)c([2H])c1[2H] ; c1ccc(-c2ccc(N(c3cccc(-c4ccc5ccccc5c4)c3)c3cc(-c4ccccc4)c4c(oc5ccccc54)c3-c3ccccc3)cc2)cc1 ; c1ccc(-c2ccc(N(c3cccc(-c4ccccc4-c4ccccc4)c3)c3cc(-c4ccccc4)c4c(oc5ccccc54)c3-c3ccccc3)cc2)cc1]

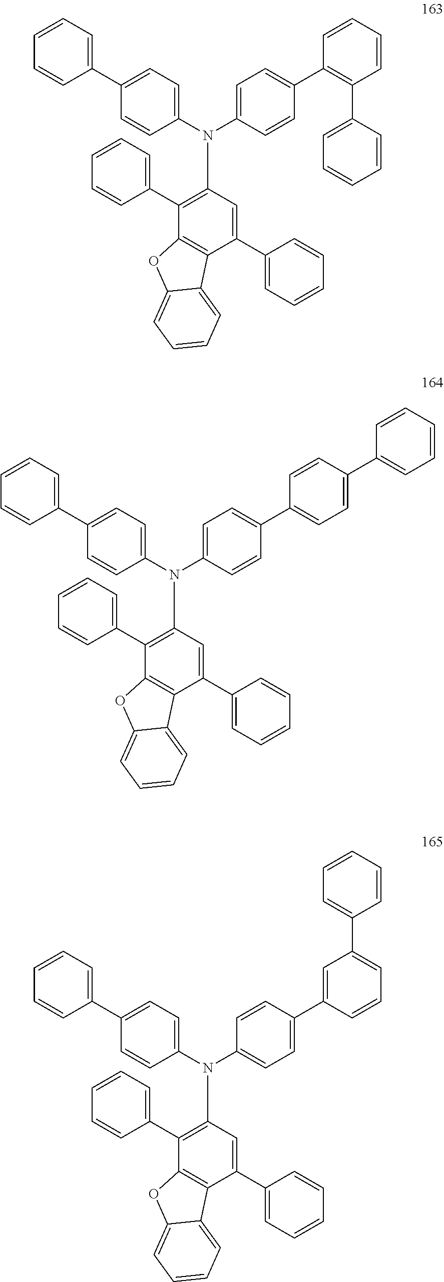 [SMILES: c1ccc(-c2ccc(N(c3ccc(-c4ccccc4-c4ccccc4)cc3)c3cc(-c4ccccc4)c4c(oc5ccccc54)c3-c3ccccc3)cc2)cc1 ; c1ccc(-c2ccc(-c3ccc(N(c4ccc(-c5ccccc5)cc4)c4cc(-c5ccccc5)c5c(oc6ccccc65)c4-c4ccccc4)cc3)cc2)cc1 ; c1ccc(-c2ccc(N(c3ccc(-c4cccc(-c5ccccc5)c4)cc3)c3cc(-c4ccccc4)c4c(oc5ccccc54)c3-c3ccccc3)cc2)cc1]

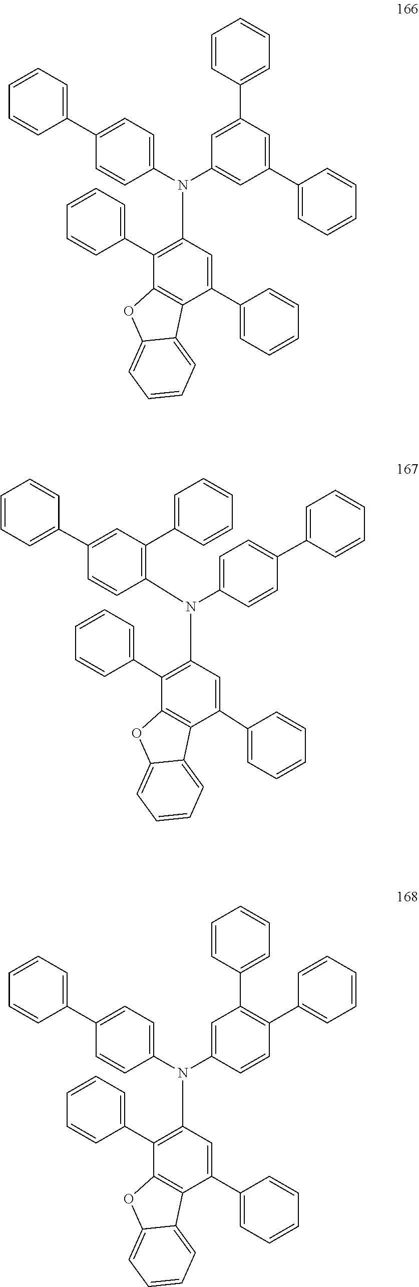 [SMILES: c1ccc(-c2ccc(N(c3cc(-c4ccccc4)cc(-c4ccccc4)c3)c3cc(-c4ccccc4)c4c(oc5ccccc54)c3-c3ccccc3)cc2)cc1 ; c1ccc(-c2ccc(N(c3ccc(-c4ccccc4)cc3-c3ccccc3)c3cc(-c4ccccc4)c4c(oc5ccccc54)c3-c3ccccc3)cc2)cc1 ; c1ccc(-c2ccc(N(c3ccc(-c4ccccc4)c(-c4ccccc4)c3)c3cc(-c4ccccc4)c4c(oc5ccccc54)c3-c3ccccc3)cc2)cc1]

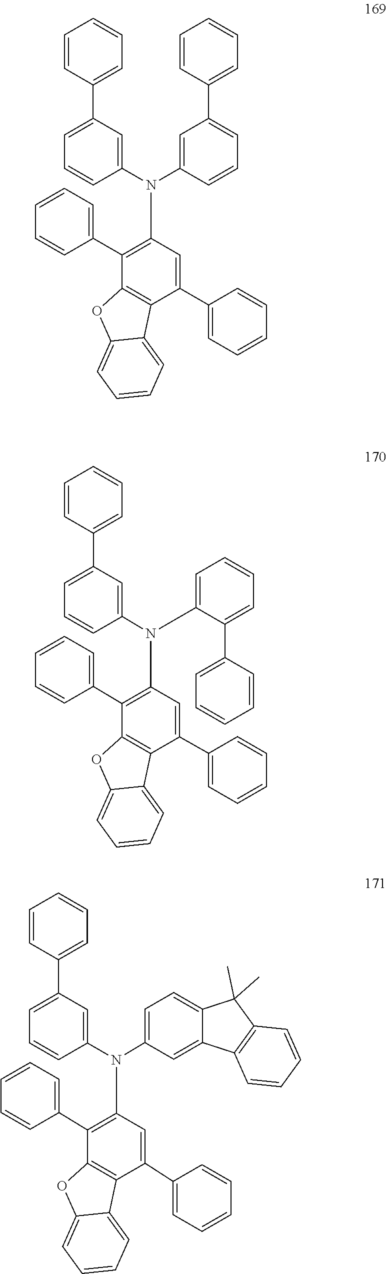 [SMILES: c1ccc(-c2cccc(N(c3cccc(-c4ccccc4)c3)c3cc(-c4ccccc4)c4c(oc5ccccc54)c3-c3ccccc3)c2)cc1 ; c1ccc(-c2cccc(N(c3ccccc3-c3ccccc3)c3cc(-c4ccccc4)c4c(oc5ccccc54)c3-c3ccccc3)c2)cc1 ; CC1(C)c2ccccc2-c2cc(N(c3cccc(-c4ccccc4)c3)c3cc(-c4ccccc4)c4c(oc5ccccc54)c3-c3ccccc3)ccc21]

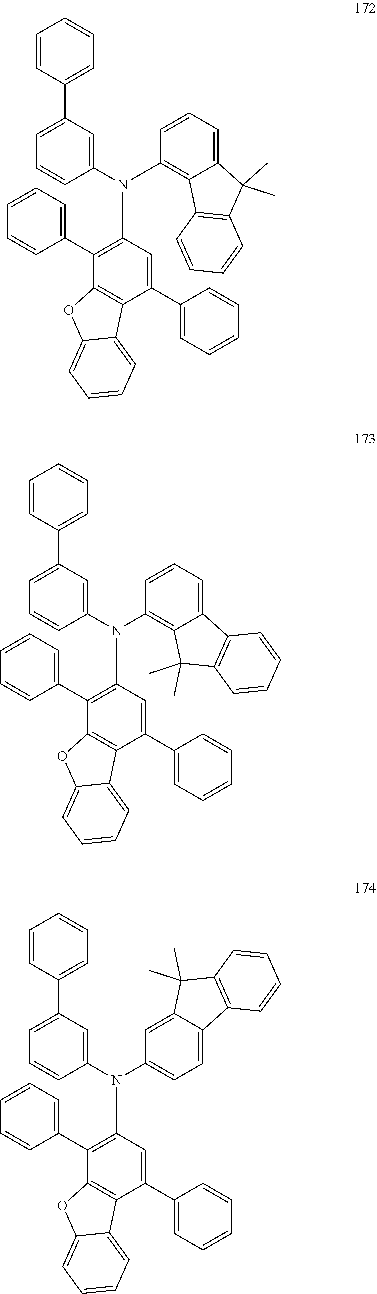 [SMILES: CC1(C)c2ccccc2-c2c(N(c3cccc(-c4ccccc4)c3)c3cc(-c4ccccc4)c4c(oc5ccccc54)c3-c3ccccc3)cccc21 ; CC1(C)c2ccccc2-c2cccc(N(c3cccc(-c4ccccc4)c3)c3cc(-c4ccccc4)c4c(oc5ccccc54)c3-c3ccccc3)c21 ; CC1(C)c2ccccc2-c2ccc(N(c3cccc(-c4ccccc4)c3)c3cc(-c4ccccc4)c4c(oc5ccccc54)c3-c3ccccc3)cc21]

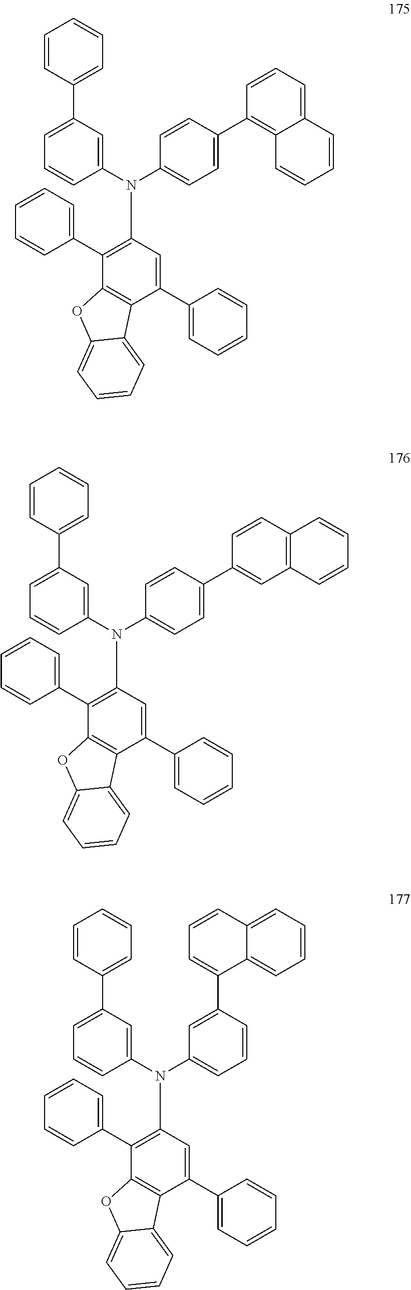 [SMILES: c1ccc(-c2cccc(N(c3ccc(-c4cccc5ccccc45)cc3)c3cc(-c4ccccc4)c4c(oc5ccccc54)c3-c3ccccc3)c2)cc1 ; c1ccc(-c2cccc(N(c3ccc(-c4ccc5ccccc5c4)cc3)c3cc(-c4ccccc4)c4c(oc5ccccc54)c3-c3ccccc3)c2)cc1 ; c1ccc(-c2cccc(N(c3cccc(-c4cccc5ccccc45)c3)c3cc(-c4ccccc4)c4c(oc5ccccc54)c3-c3ccccc3)c2)cc1]

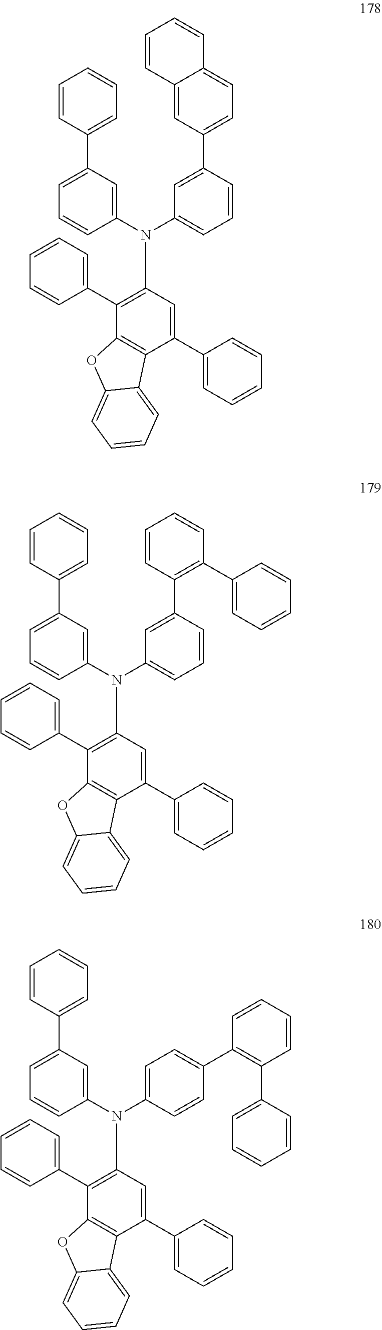 [SMILES: c1ccc(-c2cccc(N(c3cccc(-c4ccc5ccccc5c4)c3)c3cc(-c4ccccc4)c4c(oc5ccccc54)c3-c3ccccc3)c2)cc1 ; c1ccc(-c2cccc(N(c3cccc(-c4ccccc4-c4ccccc4)c3)c3cc(-c4ccccc4)c4c(oc5ccccc54)c3-c3ccccc3)c2)cc1 ; c1ccc(-c2cccc(N(c3ccc(-c4ccccc4-c4ccccc4)cc3)c3cc(-c4ccccc4)c4c(oc5ccccc54)c3-c3ccccc3)c2)cc1]

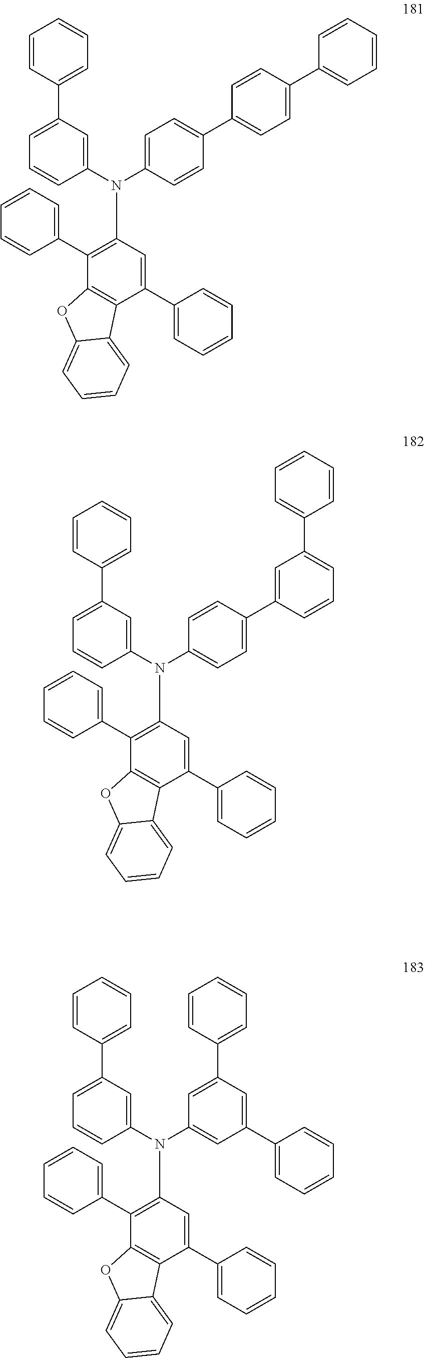 [SMILES: c1ccc(-c2ccc(-c3ccc(N(c4cccc(-c5ccccc5)c4)c4cc(-c5ccccc5)c5c(oc6ccccc65)c4-c4ccccc4)cc3)cc2)cc1 ; c1ccc(-c2cccc(-c3ccc(N(c4cccc(-c5ccccc5)c4)c4cc(-c5ccccc5)c5c(oc6ccccc65)c4-c4ccccc4)cc3)c2)cc1 ; c1ccc(-c2cccc(N(c3cc(-c4ccccc4)cc(-c4ccccc4)c3)c3cc(-c4ccccc4)c4c(oc5ccccc54)c3-c3ccccc3)c2)cc1]

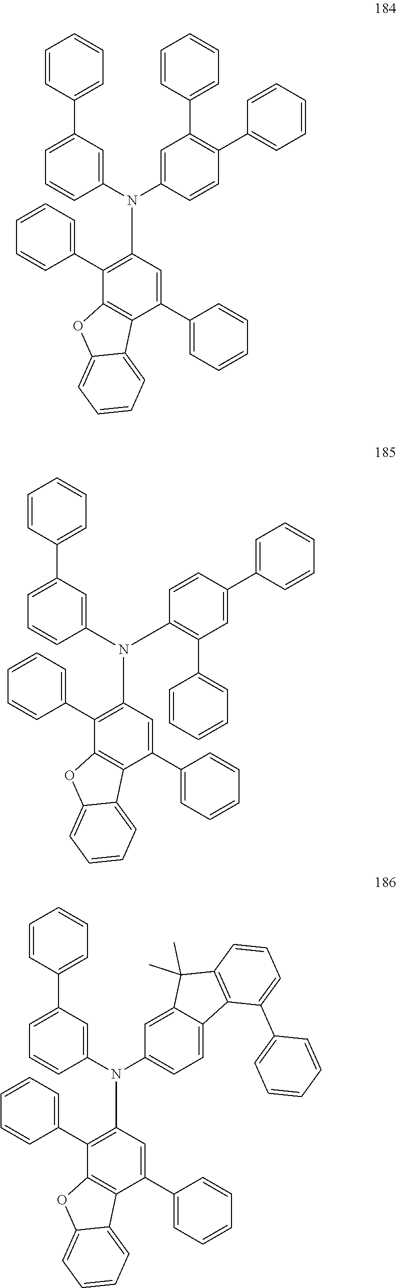 [SMILES: c1ccc(-c2cccc(N(c3ccc(-c4ccccc4)c(-c4ccccc4)c3)c3cc(-c4ccccc4)c4c(oc5ccccc54)c3-c3ccccc3)c2)cc1 ; c1ccc(-c2cccc(N(c3ccc(-c4ccccc4)cc3-c3ccccc3)c3cc(-c4ccccc4)c4c(oc5ccccc54)c3-c3ccccc3)c2)cc1 ; CC1(C)c2cc(N(c3cccc(-c4ccccc4)c3)c3cc(-c4ccccc4)c4c(oc5ccccc54)c3-c3ccccc3)ccc2-c2c(-c3ccccc3)cccc21]

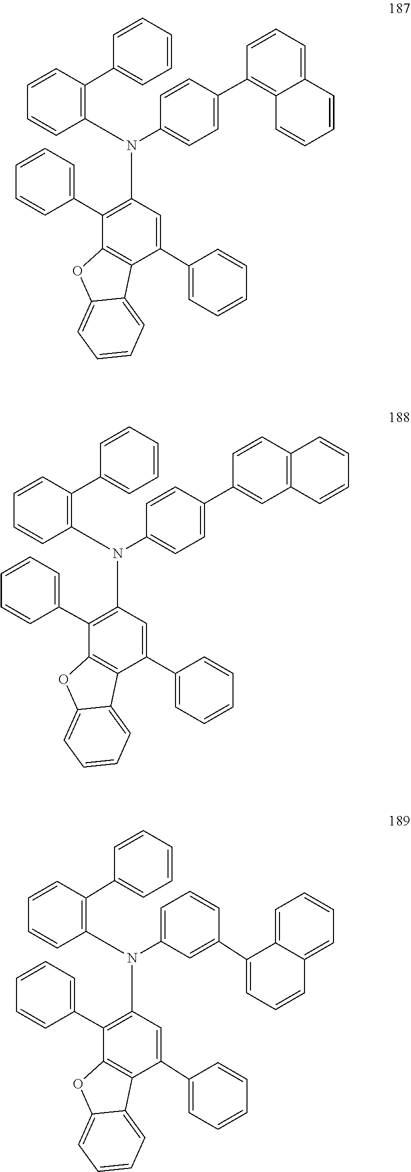 [SMILES: c1ccc(-c2ccccc2N(c2ccc(-c3cccc4ccccc34)cc2)c2cc(-c3ccccc3)c3c(oc4ccccc43)c2-c2ccccc2)cc1 ; c1ccc(-c2ccccc2N(c2ccc(-c3ccc4ccccc4c3)cc2)c2cc(-c3ccccc3)c3c(oc4ccccc43)c2-c2ccccc2)cc1 ; c1ccc(-c2ccccc2N(c2cccc(-c3cccc4ccccc34)c2)c2cc(-c3ccccc3)c3c(oc4ccccc43)c2-c2ccccc2)cc1]

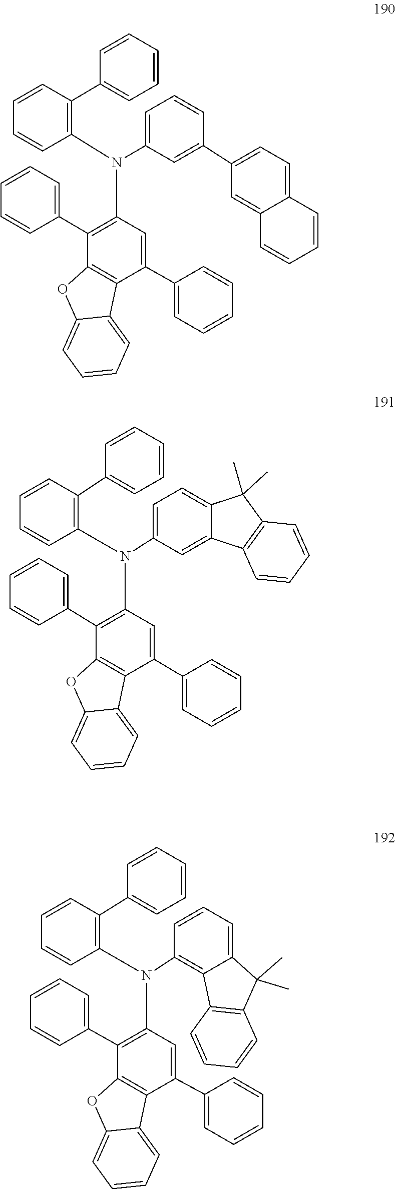 [SMILES: c1ccc(-c2ccccc2N(c2cccc(-c3ccc4ccccc4c3)c2)c2cc(-c3ccccc3)c3c(oc4ccccc43)c2-c2ccccc2)cc1 ; CC1(C)c2ccccc2-c2cc(N(c3ccccc3-c3ccccc3)c3cc(-c4ccccc4)c4c(oc5ccccc54)c3-c3ccccc3)ccc21 ; CC1(C)c2ccccc2-c2c(N(c3ccccc3-c3ccccc3)c3cc(-c4ccccc4)c4c(oc5ccccc54)c3-c3ccccc3)cccc21]

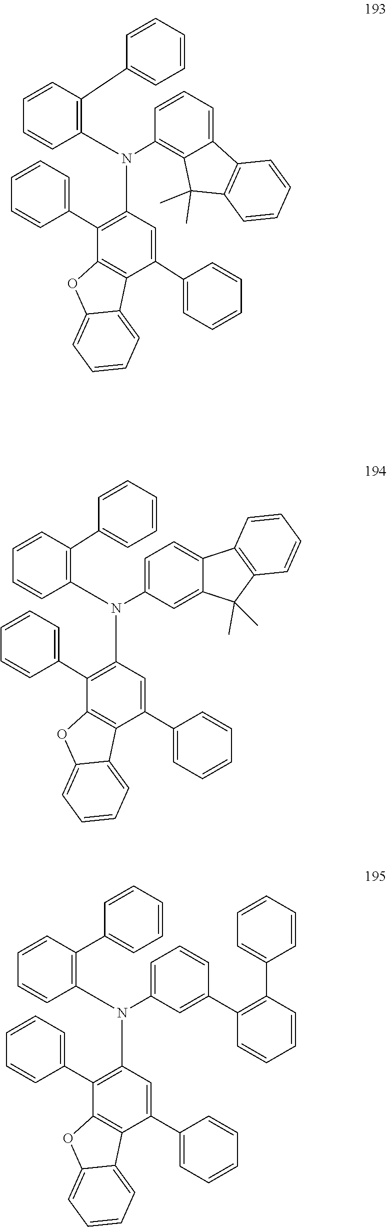 [SMILES: CC1(C)c2ccccc2-c2cccc(N(c3ccccc3-c3ccccc3)c3cc(-c4ccccc4)c4c(oc5ccccc54)c3-c3ccccc3)c21 ; CC1(C)c2ccccc2-c2ccc(N(c3ccccc3-c3ccccc3)c3cc(-c4ccccc4)c4c(oc5ccccc54)c3-c3ccccc3)cc21 ; c1ccc(-c2ccccc2-c2cccc(N(c3ccccc3-c3ccccc3)c3cc(-c4ccccc4)c4c(oc5ccccc54)c3-c3ccccc3)c2)cc1]

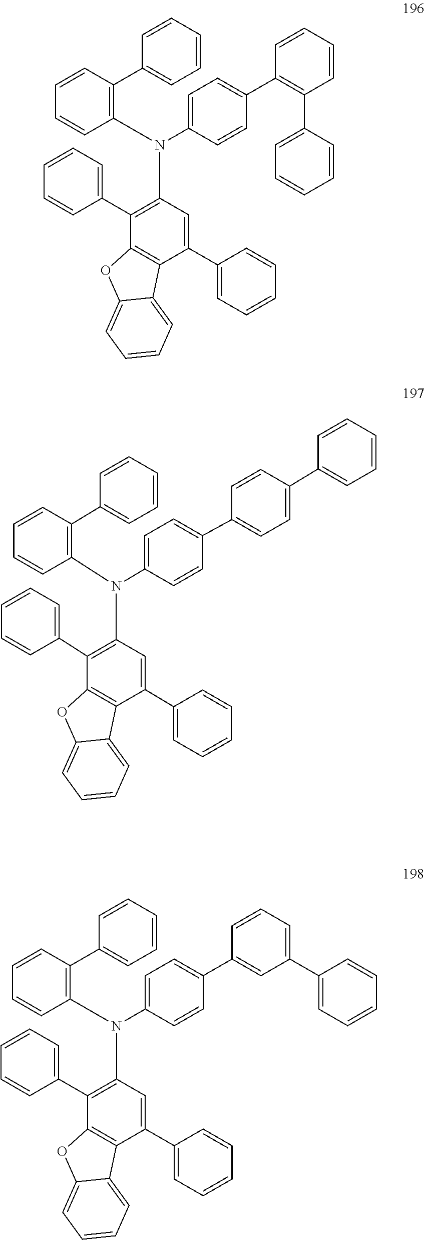 [SMILES: c1ccc(-c2ccccc2-c2ccc(N(c3ccccc3-c3ccccc3)c3cc(-c4ccccc4)c4c(oc5ccccc54)c3-c3ccccc3)cc2)cc1 ; c1ccc(-c2ccc(-c3ccc(N(c4ccccc4-c4ccccc4)c4cc(-c5ccccc5)c5c(oc6ccccc65)c4-c4ccccc4)cc3)cc2)cc1 ; c1ccc(-c2cccc(-c3ccc(N(c4ccccc4-c4ccccc4)c4cc(-c5ccccc5)c5c(oc6ccccc65)c4-c4ccccc4)cc3)c2)cc1]

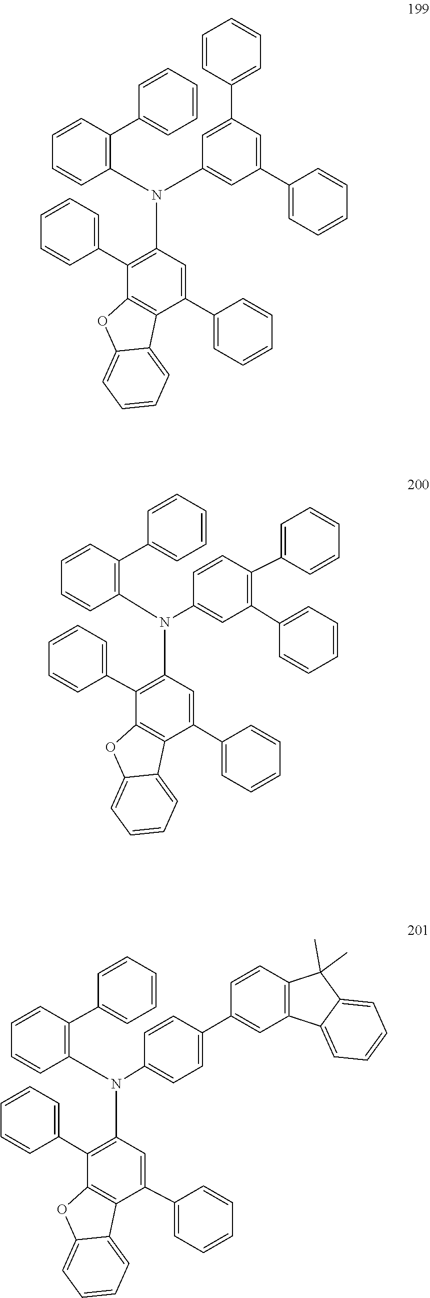 [SMILES: c1ccc(-c2cc(-c3ccccc3)cc(N(c3ccccc3-c3ccccc3)c3cc(-c4ccccc4)c4c(oc5ccccc54)c3-c3ccccc3)c2)cc1 ; c1ccc(-c2ccc(N(c3ccccc3-c3ccccc3)c3cc(-c4ccccc4)c4c(oc5ccccc54)c3-c3ccccc3)cc2-c2ccccc2)cc1 ; CC1(C)c2ccccc2-c2cc(-c3ccc(N(c4ccccc4-c4ccccc4)c4cc(-c5ccccc5)c5c(oc6ccccc65)c4-c4ccccc4)cc3)ccc21]

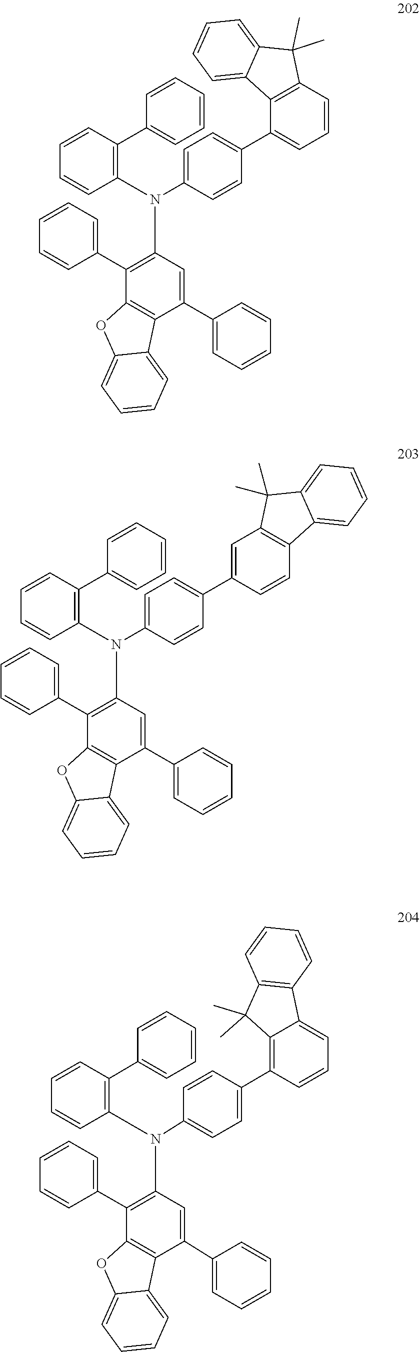 [SMILES: CC1(C)c2ccccc2-c2c(-c3ccc(N(c4ccccc4-c4ccccc4)c4cc(-c5ccccc5)c5c(oc6ccccc65)c4-c4ccccc4)cc3)cccc21 ; CC1(C)c2ccccc2-c2ccc(-c3ccc(N(c4ccccc4-c4ccccc4)c4cc(-c5ccccc5)c5c(oc6ccccc65)c4-c4ccccc4)cc3)cc21 ; CC1(C)c2ccccc2-c2cccc(-c3ccc(N(c4ccccc4-c4ccccc4)c4cc(-c5ccccc5)c5c(oc6ccccc65)c4-c4ccccc4)cc3)c21]

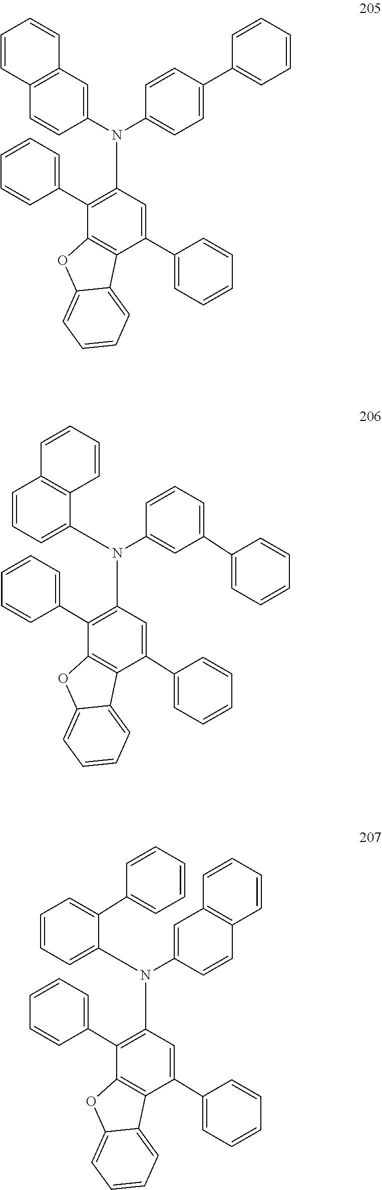 [SMILES: c1ccc(-c2ccc(N(c3ccc4ccccc4c3)c3cc(-c4ccccc4)c4c(oc5ccccc54)c3-c3ccccc3)cc2)cc1 ; c1ccc(-c2cccc(N(c3cc(-c4ccccc4)c4c(oc5ccccc54)c3-c3ccccc3)c3cccc4ccccc34)c2)cc1 ; c1ccc(-c2ccccc2N(c2ccc3ccccc3c2)c2cc(-c3ccccc3)c3c(oc4ccccc43)c2-c2ccccc2)cc1]

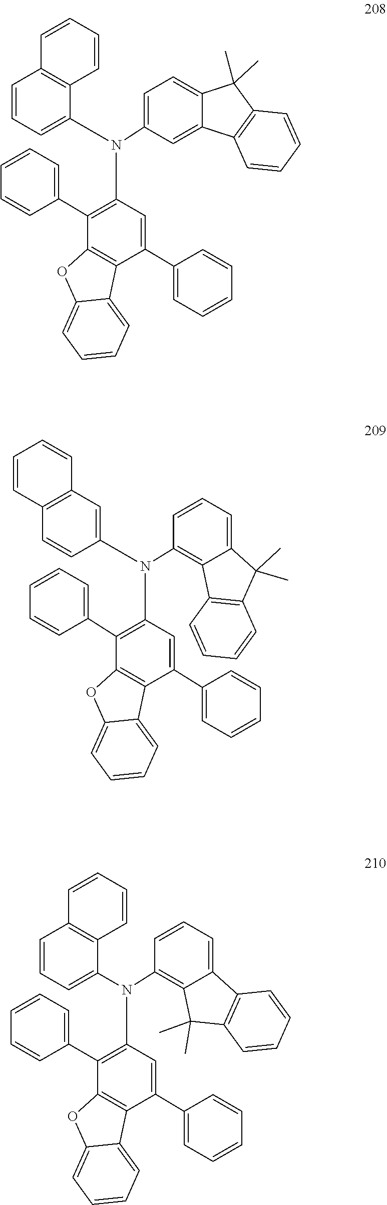 [SMILES: CC1(C)c2ccccc2-c2cc(N(c3cc(-c4ccccc4)c4c(oc5ccccc54)c3-c3ccccc3)c3cccc4ccccc34)ccc21 ; CC1(C)c2ccccc2-c2c(N(c3ccc4ccccc4c3)c3cc(-c4ccccc4)c4c(oc5ccccc54)c3-c3ccccc3)cccc21 ; CC1(C)c2ccccc2-c2cccc(N(c3cc(-c4ccccc4)c4c(oc5ccccc54)c3-c3ccccc3)c3cccc4ccccc34)c21]

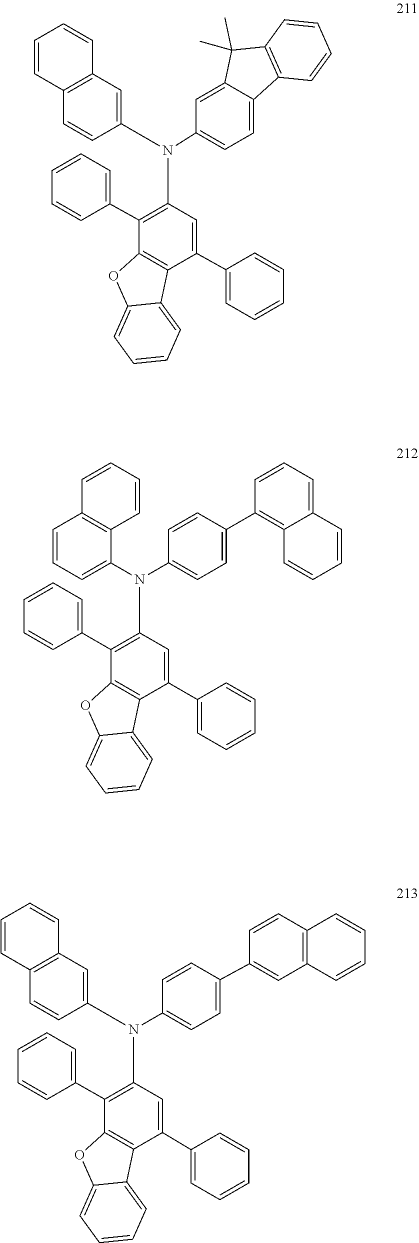 [SMILES: CC1(C)c2ccccc2-c2ccc(N(c3ccc4ccccc4c3)c3cc(-c4ccccc4)c4c(oc5ccccc54)c3-c3ccccc3)cc21 ; c1ccc(-c2c(N(c3ccc(-c4cccc5ccccc45)cc3)c3cccc4ccccc34)cc(-c3ccccc3)c3c2oc2ccccc23)cc1 ; c1ccc(-c2c(N(c3ccc(-c4ccc5ccccc5c4)cc3)c3ccc4ccccc4c3)cc(-c3ccccc3)c3c2oc2ccccc23)cc1]

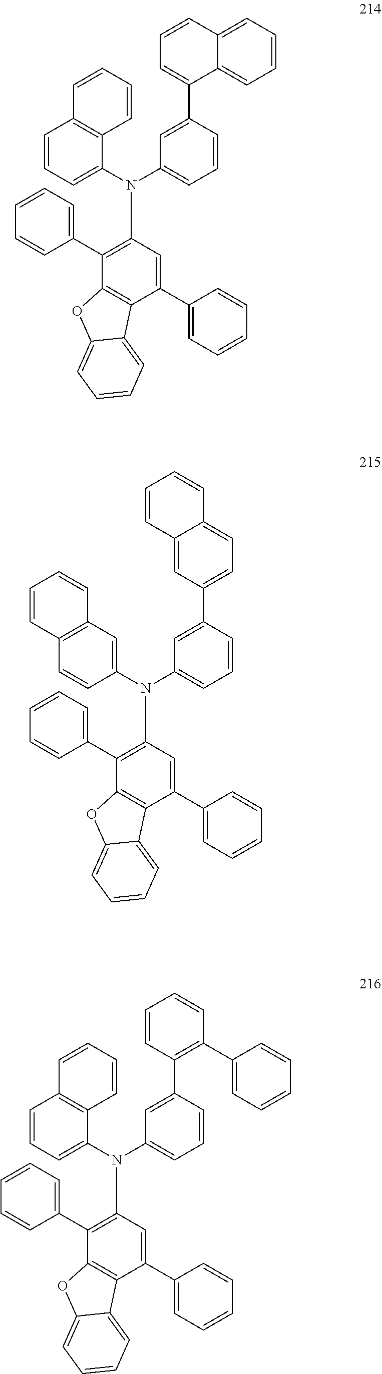 [SMILES: c1ccc(-c2c(N(c3cccc(-c4cccc5ccccc45)c3)c3cccc4ccccc34)cc(-c3ccccc3)c3c2oc2ccccc23)cc1 ; c1ccc(-c2c(N(c3cccc(-c4ccc5ccccc5c4)c3)c3ccc4ccccc4c3)cc(-c3ccccc3)c3c2oc2ccccc23)cc1 ; c1ccc(-c2ccccc2-c2cccc(N(c3cc(-c4ccccc4)c4c(oc5ccccc54)c3-c3ccccc3)c3cccc4ccccc34)c2)cc1]

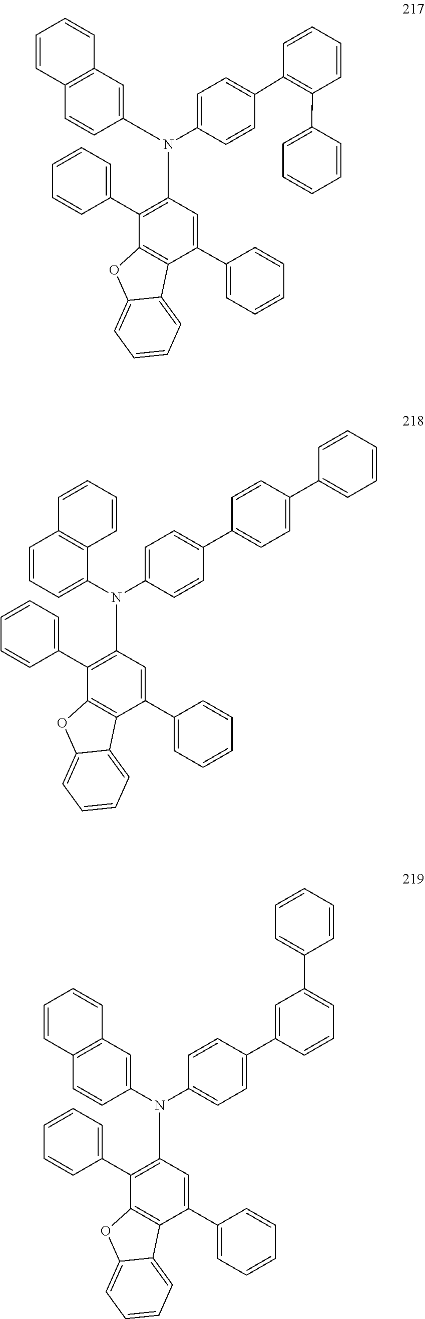 [SMILES: c1ccc(-c2ccccc2-c2ccc(N(c3ccc4ccccc4c3)c3cc(-c4ccccc4)c4c(oc5ccccc54)c3-c3ccccc3)cc2)cc1 ; c1ccc(-c2ccc(-c3ccc(N(c4cc(-c5ccccc5)c5c(oc6ccccc65)c4-c4ccccc4)c4cccc5ccccc45)cc3)cc2)cc1 ; c1ccc(-c2cccc(-c3ccc(N(c4ccc5ccccc5c4)c4cc(-c5ccccc5)c5c(oc6ccccc65)c4-c4ccccc4)cc3)c2)cc1]

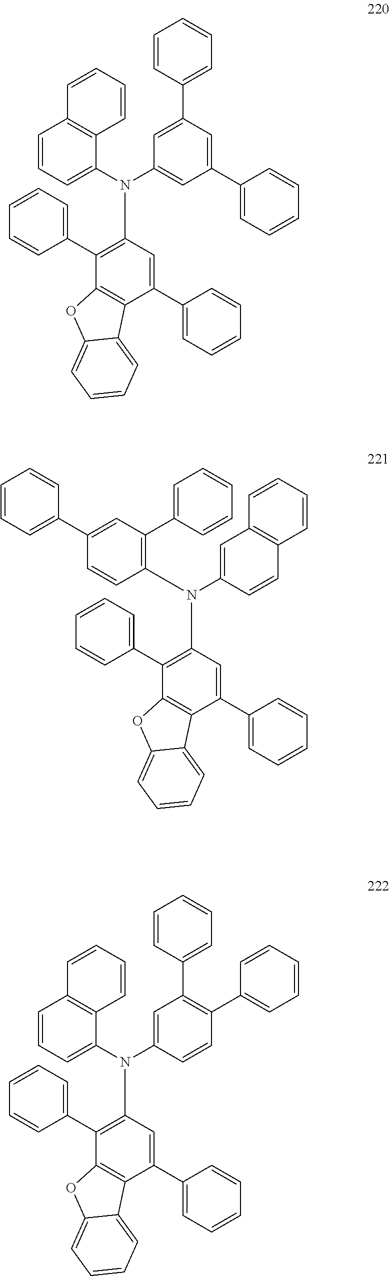 [SMILES: c1ccc(-c2cc(-c3ccccc3)cc(N(c3cc(-c4ccccc4)c4c(oc5ccccc54)c3-c3ccccc3)c3cccc4ccccc34)c2)cc1 ; c1ccc(-c2ccc(N(c3ccc4ccccc4c3)c3cc(-c4ccccc4)c4c(oc5ccccc54)c3-c3ccccc3)c(-c3ccccc3)c2)cc1 ; c1ccc(-c2ccc(N(c3cc(-c4ccccc4)c4c(oc5ccccc54)c3-c3ccccc3)c3cccc4ccccc34)cc2-c2ccccc2)cc1]

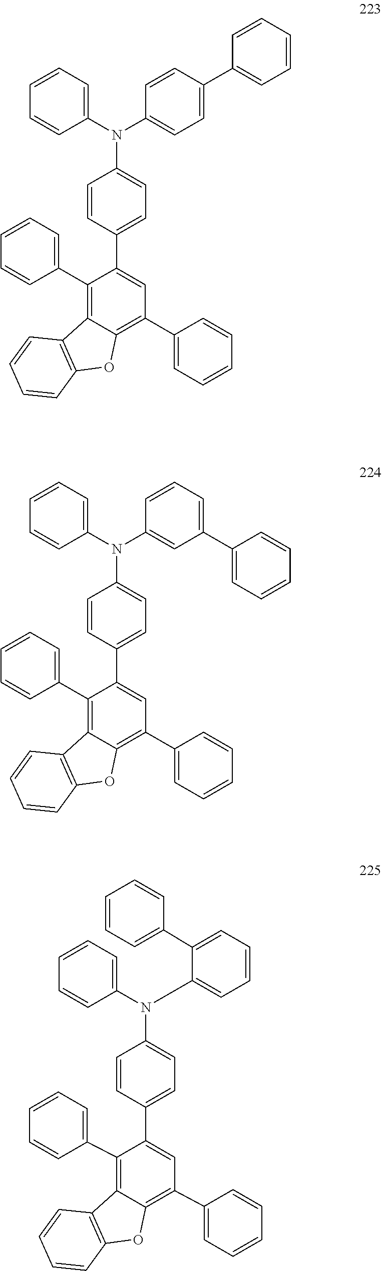 [SMILES: c1ccc(-c2ccc(N(c3ccccc3)c3ccc(-c4cc(-c5ccccc5)c5oc6ccccc6c5c4-c4ccccc4)cc3)cc2)cc1 ; c1ccc(-c2cccc(N(c3ccccc3)c3ccc(-c4cc(-c5ccccc5)c5oc6ccccc6c5c4-c4ccccc4)cc3)c2)cc1 ; c1ccc(-c2ccccc2N(c2ccccc2)c2ccc(-c3cc(-c4ccccc4)c4oc5ccccc5c4c3-c3ccccc3)cc2)cc1]

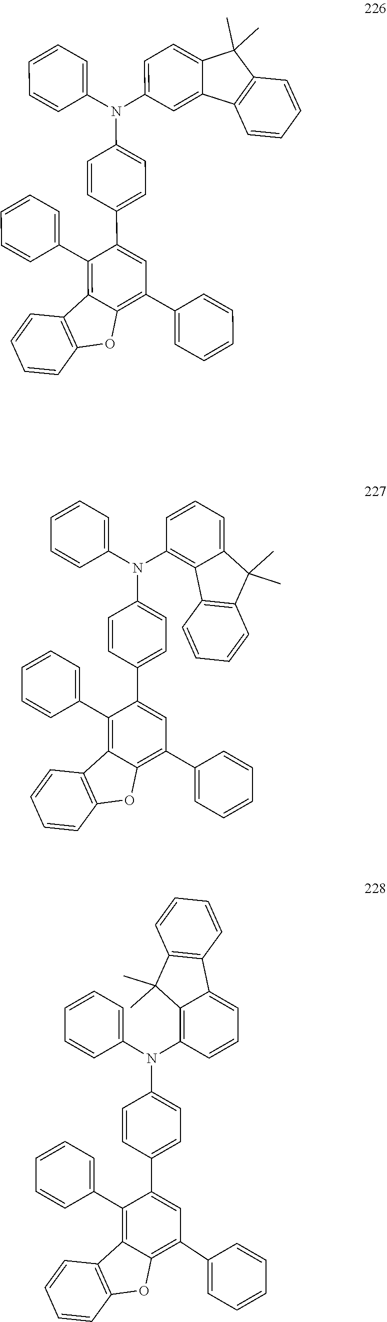 [SMILES: CC1(C)c2ccccc2-c2cc(N(c3ccccc3)c3ccc(-c4cc(-c5ccccc5)c5oc6ccccc6c5c4-c4ccccc4)cc3)ccc21 ; CC1(C)c2ccccc2-c2c(N(c3ccccc3)c3ccc(-c4cc(-c5ccccc5)c5oc6ccccc6c5c4-c4ccccc4)cc3)cccc21 ; CC1(C)c2ccccc2-c2cccc(N(c3ccccc3)c3ccc(-c4cc(-c5ccccc5)c5oc6ccccc6c5c4-c4ccccc4)cc3)c21]

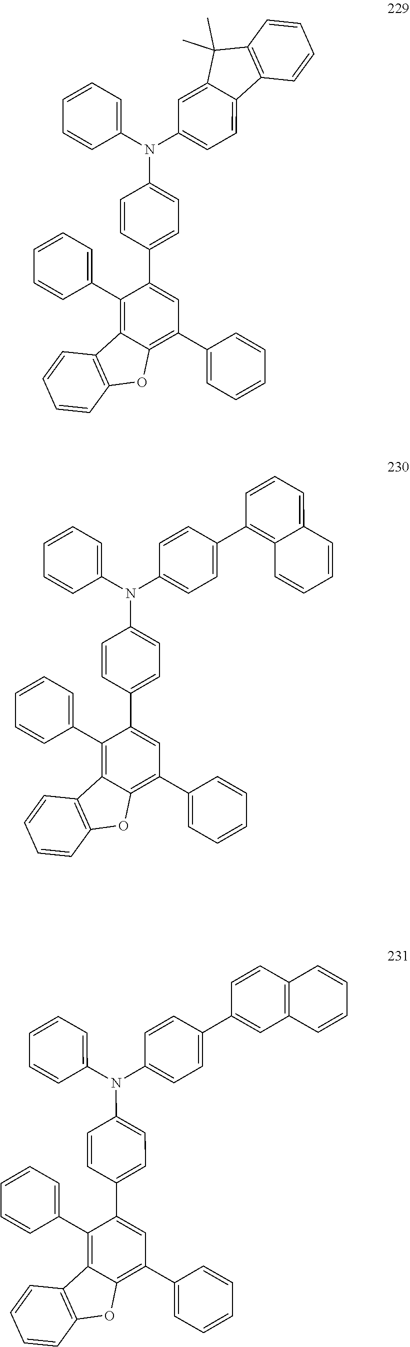 [SMILES: CC1(C)c2ccccc2-c2ccc(N(c3ccccc3)c3ccc(-c4cc(-c5ccccc5)c5oc6ccccc6c5c4-c4ccccc4)cc3)cc21 ; c1ccc(-c2cc(-c3ccc(N(c4ccccc4)c4ccc(-c5cccc6ccccc56)cc4)cc3)c(-c3ccccc3)c3c2oc2ccccc23)cc1 ; c1ccc(-c2cc(-c3ccc(N(c4ccccc4)c4ccc(-c5ccc6ccccc6c5)cc4)cc3)c(-c3ccccc3)c3c2oc2ccccc23)cc1]

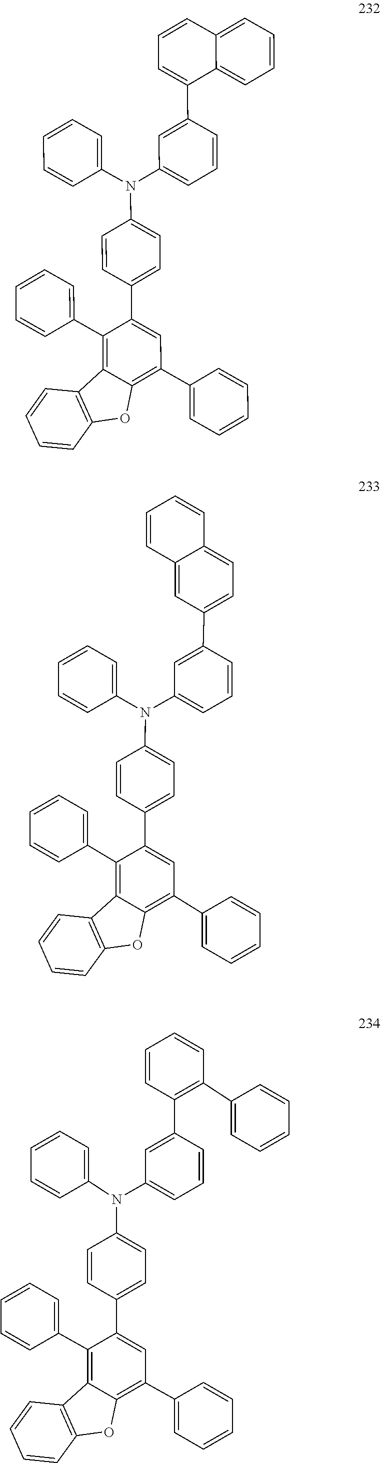 [SMILES: c1ccc(-c2cc(-c3ccc(N(c4ccccc4)c4cccc(-c5cccc6ccccc56)c4)cc3)c(-c3ccccc3)c3c2oc2ccccc23)cc1 ; c1ccc(-c2cc(-c3ccc(N(c4ccccc4)c4cccc(-c5ccc6ccccc6c5)c4)cc3)c(-c3ccccc3)c3c2oc2ccccc23)cc1 ; c1ccc(-c2ccccc2-c2cccc(N(c3ccccc3)c3ccc(-c4cc(-c5ccccc5)c5oc6ccccc6c5c4-c4ccccc4)cc3)c2)cc1]

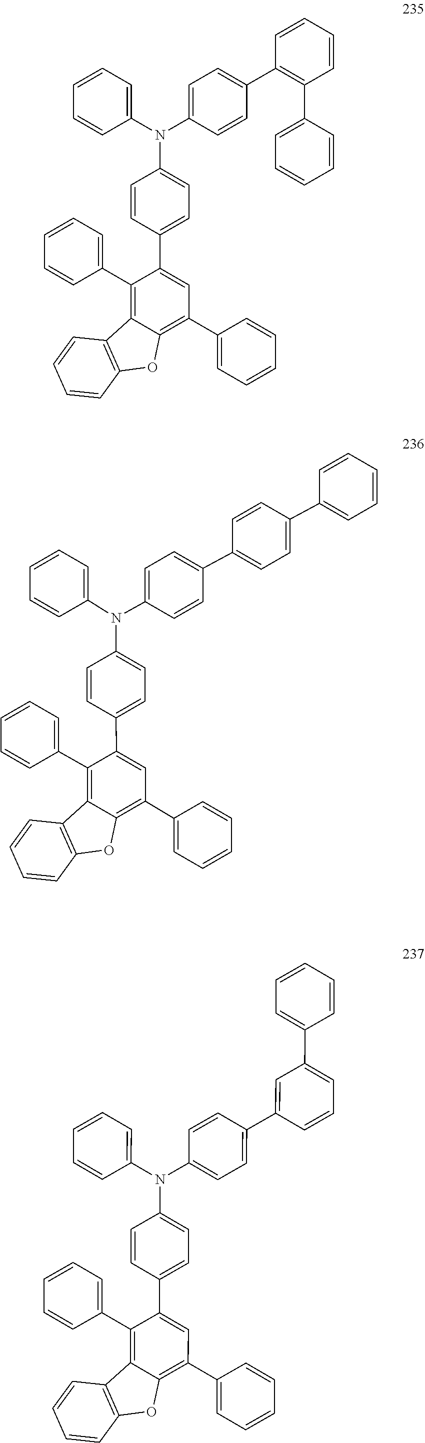 [SMILES: c1ccc(-c2ccccc2-c2ccc(N(c3ccccc3)c3ccc(-c4cc(-c5ccccc5)c5oc6ccccc6c5c4-c4ccccc4)cc3)cc2)cc1 ; c1ccc(-c2ccc(-c3ccc(N(c4ccccc4)c4ccc(-c5cc(-c6ccccc6)c6oc7ccccc7c6c5-c5ccccc5)cc4)cc3)cc2)cc1 ; c1ccc(-c2cccc(-c3ccc(N(c4ccccc4)c4ccc(-c5cc(-c6ccccc6)c6oc7ccccc7c6c5-c5ccccc5)cc4)cc3)c2)cc1]

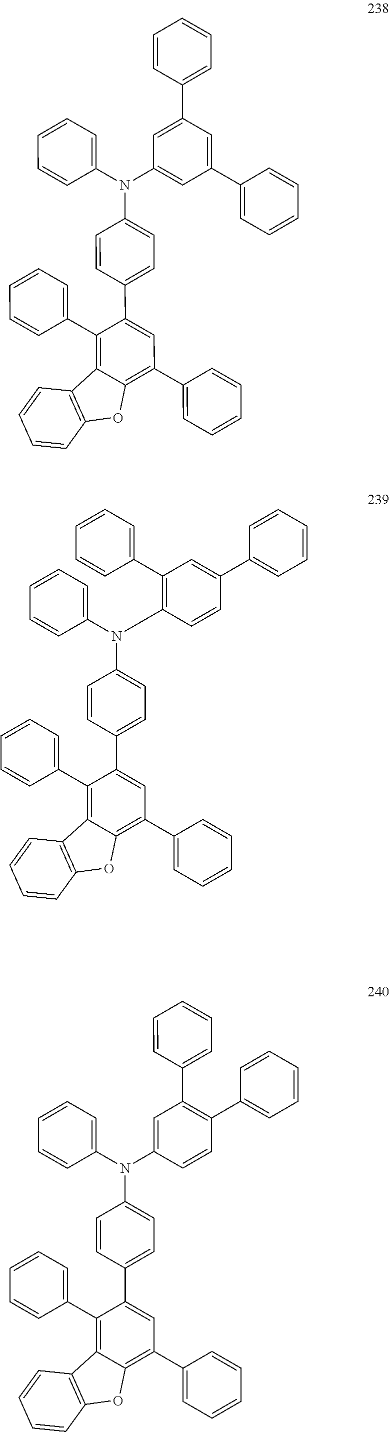 [SMILES: c1ccc(-c2cc(-c3ccccc3)cc(N(c3ccccc3)c3ccc(-c4cc(-c5ccccc5)c5oc6ccccc6c5c4-c4ccccc4)cc3)c2)cc1 ; c1ccc(-c2ccc(N(c3ccccc3)c3ccc(-c4cc(-c5ccccc5)c5oc6ccccc6c5c4-c4ccccc4)cc3)c(-c3ccccc3)c2)cc1 ; c1ccc(-c2ccc(N(c3ccccc3)c3ccc(-c4cc(-c5ccccc5)c5oc6ccccc6c5c4-c4ccccc4)cc3)cc2-c2ccccc2)cc1]

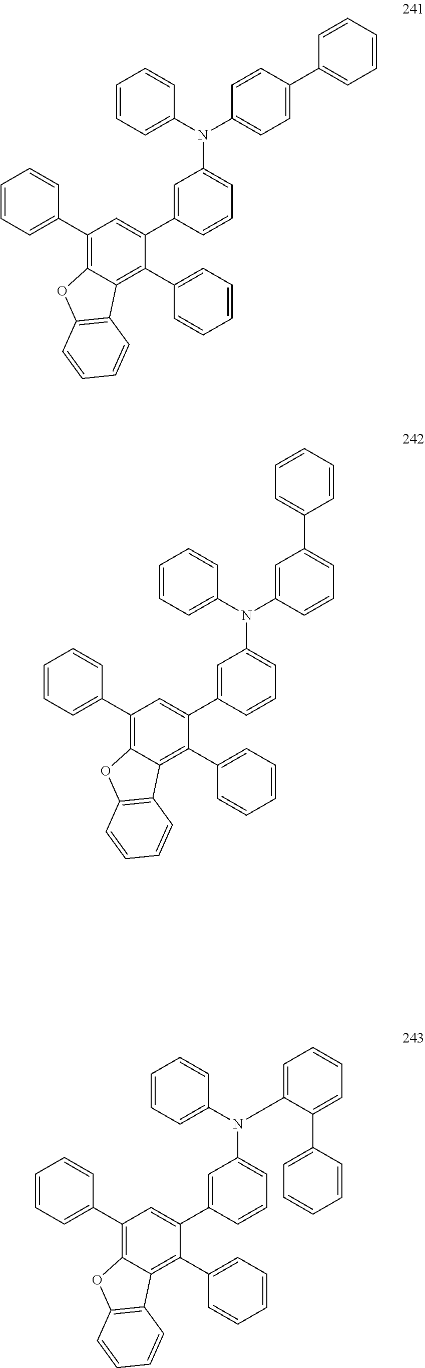 [SMILES: c1ccc(-c2ccc(N(c3ccccc3)c3cccc(-c4cc(-c5ccccc5)c5oc6ccccc6c5c4-c4ccccc4)c3)cc2)cc1 ; c1ccc(-c2cccc(N(c3ccccc3)c3cccc(-c4cc(-c5ccccc5)c5oc6ccccc6c5c4-c4ccccc4)c3)c2)cc1 ; c1ccc(-c2ccccc2N(c2ccccc2)c2cccc(-c3cc(-c4ccccc4)c4oc5ccccc5c4c3-c3ccccc3)c2)cc1]

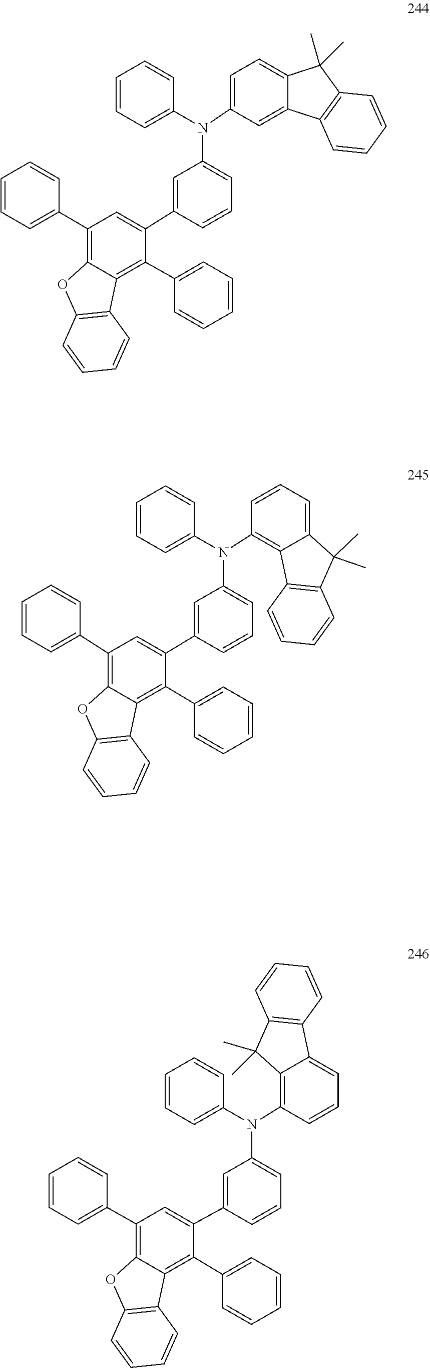 [SMILES: CC1(C)c2ccccc2-c2cc(N(c3ccccc3)c3cccc(-c4cc(-c5ccccc5)c5oc6ccccc6c5c4-c4ccccc4)c3)ccc21 ; CC1(C)c2ccccc2-c2c(N(c3ccccc3)c3cccc(-c4cc(-c5ccccc5)c5oc6ccccc6c5c4-c4ccccc4)c3)cccc21 ; CC1(C)c2ccccc2-c2cccc(N(c3ccccc3)c3cccc(-c4cc(-c5ccccc5)c5oc6ccccc6c5c4-c4ccccc4)c3)c21]

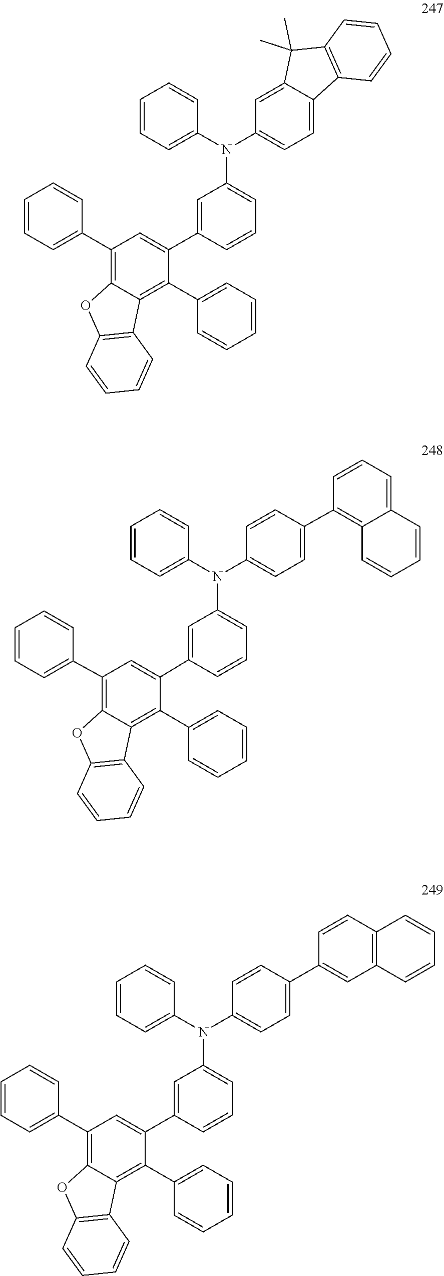 [SMILES: CC1(C)c2ccccc2-c2ccc(N(c3ccccc3)c3cccc(-c4cc(-c5ccccc5)c5oc6ccccc6c5c4-c4ccccc4)c3)cc21 ; c1ccc(-c2cc(-c3cccc(N(c4ccccc4)c4ccc(-c5cccc6ccccc56)cc4)c3)c(-c3ccccc3)c3c2oc2ccccc23)cc1 ; c1ccc(-c2cc(-c3cccc(N(c4ccccc4)c4ccc(-c5ccc6ccccc6c5)cc4)c3)c(-c3ccccc3)c3c2oc2ccccc23)cc1]

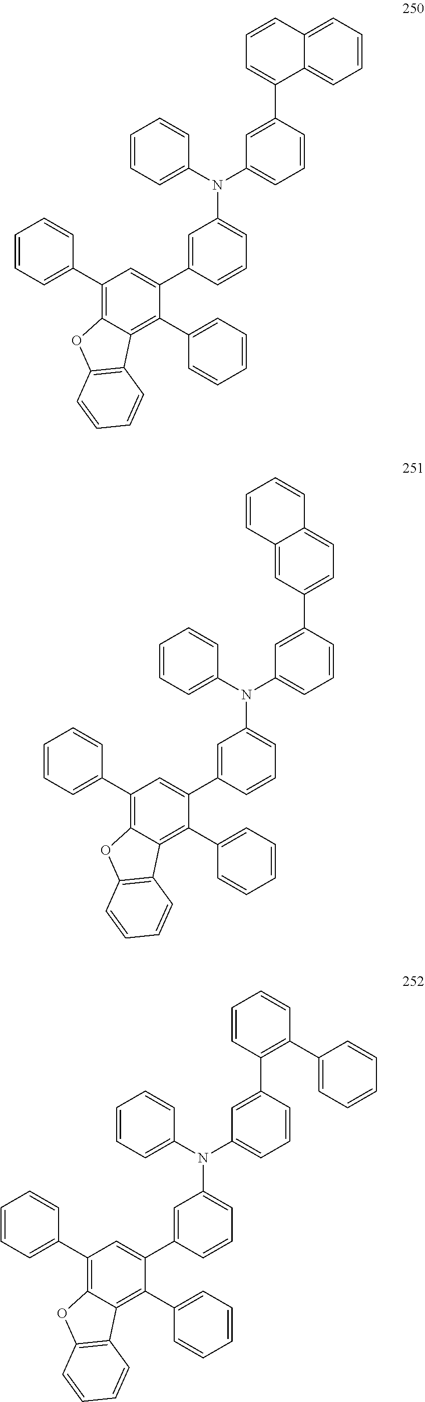 [SMILES: c1ccc(-c2cc(-c3cccc(N(c4ccccc4)c4cccc(-c5cccc6ccccc56)c4)c3)c(-c3ccccc3)c3c2oc2ccccc23)cc1 ; c1ccc(-c2cc(-c3cccc(N(c4ccccc4)c4cccc(-c5ccc6ccccc6c5)c4)c3)c(-c3ccccc3)c3c2oc2ccccc23)cc1 ; c1ccc(-c2ccccc2-c2cccc(N(c3ccccc3)c3cccc(-c4cc(-c5ccccc5)c5oc6ccccc6c5c4-c4ccccc4)c3)c2)cc1]

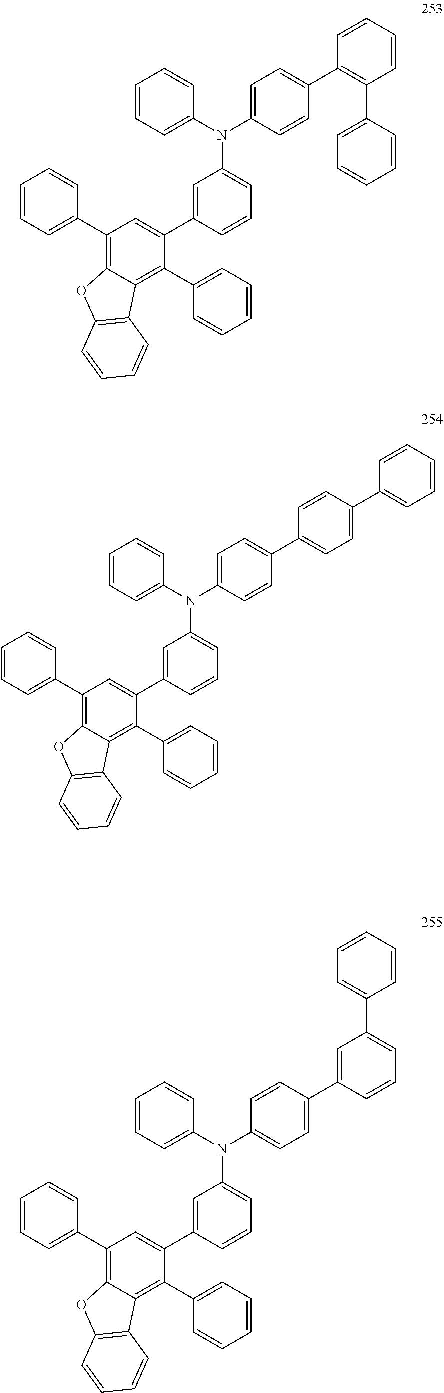 [SMILES: c1ccc(-c2ccccc2-c2ccc(N(c3ccccc3)c3cccc(-c4cc(-c5ccccc5)c5oc6ccccc6c5c4-c4ccccc4)c3)cc2)cc1 ; c1ccc(-c2ccc(-c3ccc(N(c4ccccc4)c4cccc(-c5cc(-c6ccccc6)c6oc7ccccc7c6c5-c5ccccc5)c4)cc3)cc2)cc1 ; c1ccc(-c2cccc(-c3ccc(N(c4ccccc4)c4cccc(-c5cc(-c6ccccc6)c6oc7ccccc7c6c5-c5ccccc5)c4)cc3)c2)cc1]

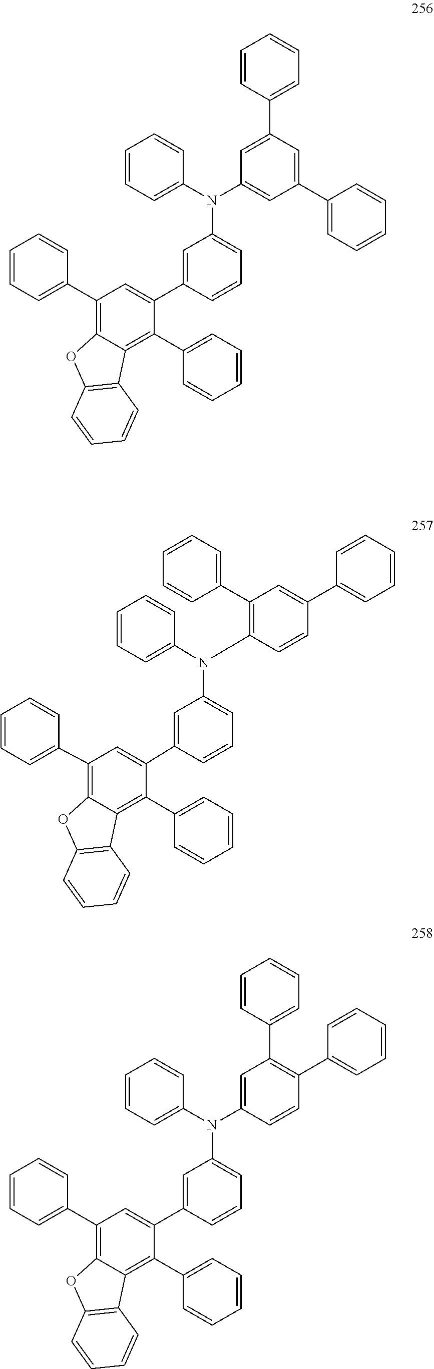 [SMILES: c1ccc(-c2cc(-c3ccccc3)cc(N(c3ccccc3)c3cccc(-c4cc(-c5ccccc5)c5oc6ccccc6c5c4-c4ccccc4)c3)c2)cc1 ; c1ccc(-c2ccc(N(c3ccccc3)c3cccc(-c4cc(-c5ccccc5)c5oc6ccccc6c5c4-c4ccccc4)c3)c(-c3ccccc3)c2)cc1 ; c1ccc(-c2ccc(N(c3ccccc3)c3cccc(-c4cc(-c5ccccc5)c5oc6ccccc6c5c4-c4ccccc4)c3)cc2-c2ccccc2)cc1]

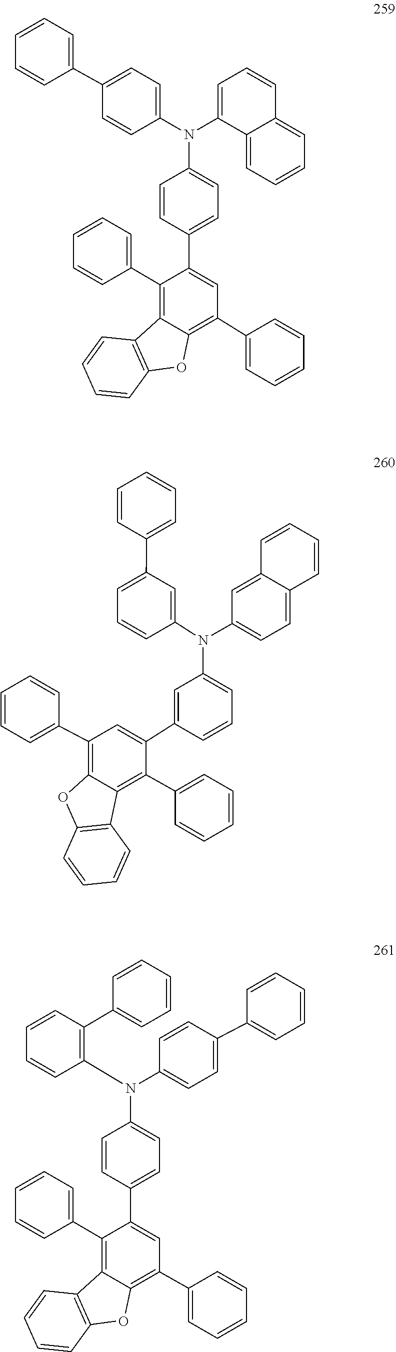 [SMILES: c1ccc(-c2ccc(N(c3ccc(-c4cc(-c5ccccc5)c5oc6ccccc6c5c4-c4ccccc4)cc3)c3cccc4ccccc34)cc2)cc1 ; c1ccc(-c2cccc(N(c3cccc(-c4cc(-c5ccccc5)c5oc6ccccc6c5c4-c4ccccc4)c3)c3ccc4ccccc4c3)c2)cc1 ; c1ccc(-c2ccc(N(c3ccc(-c4cc(-c5ccccc5)c5oc6ccccc6c5c4-c4ccccc4)cc3)c3ccccc3-c3ccccc3)cc2)cc1]

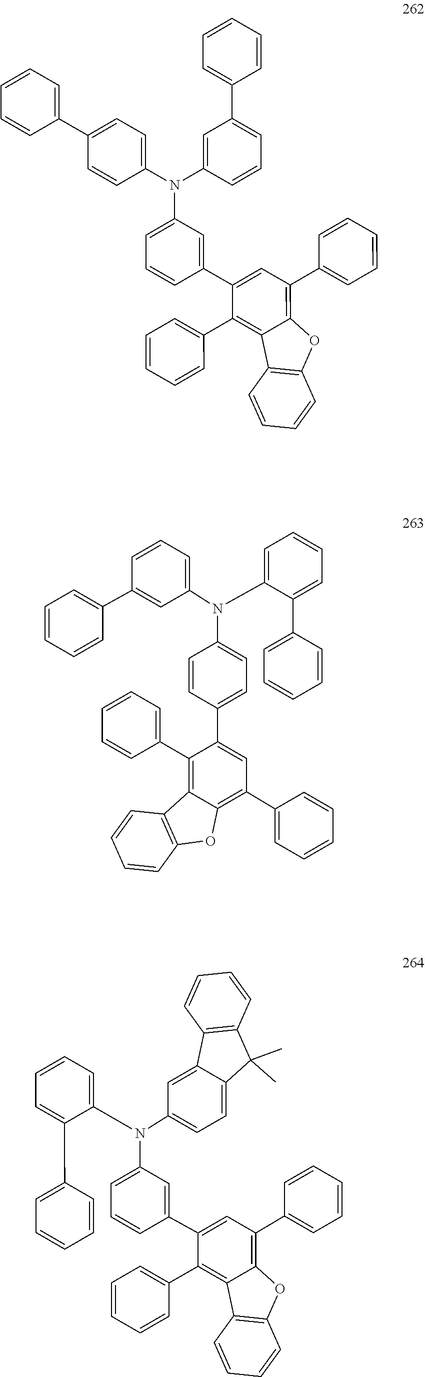 [SMILES: c1ccc(-c2ccc(N(c3cccc(-c4ccccc4)c3)c3cccc(-c4cc(-c5ccccc5)c5oc6ccccc6c5c4-c4ccccc4)c3)cc2)cc1 ; c1ccc(-c2cccc(N(c3ccc(-c4cc(-c5ccccc5)c5oc6ccccc6c5c4-c4ccccc4)cc3)c3ccccc3-c3ccccc3)c2)cc1 ; CC1(C)c2ccccc2-c2cc(N(c3cccc(-c4cc(-c5ccccc5)c5oc6ccccc6c5c4-c4ccccc4)c3)c3ccccc3-c3ccccc3)ccc21]

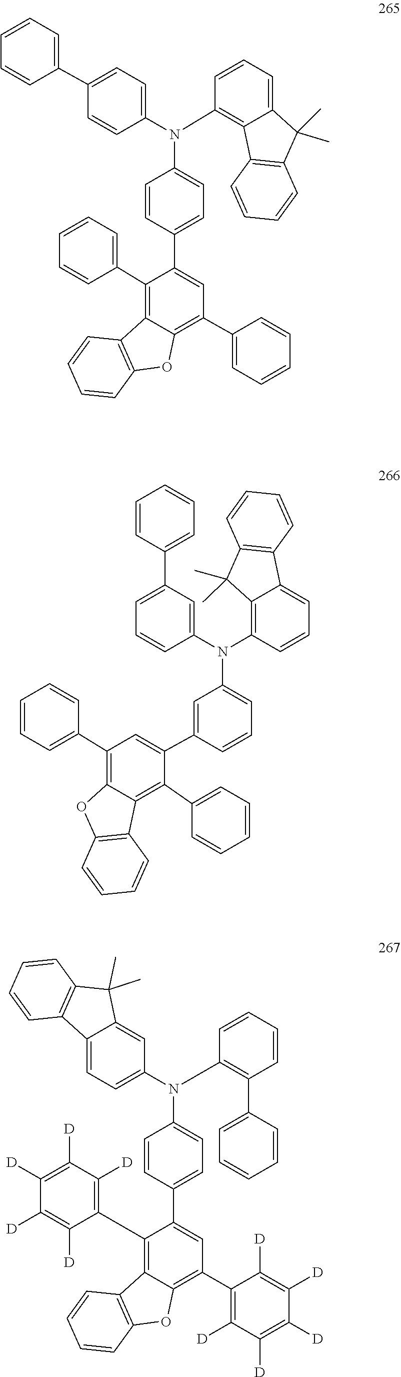 [SMILES: CC1(C)c2ccccc2-c2c(N(c3ccc(-c4ccccc4)cc3)c3ccc(-c4cc(-c5ccccc5)c5oc6ccccc6c5c4-c4ccccc4)cc3)cccc21 ; CC1(C)c2ccccc2-c2cccc(N(c3cccc(-c4ccccc4)c3)c3cccc(-c4cc(-c5ccccc5)c5oc6ccccc6c5c4-c4ccccc4)c3)c21 ; [2H]c1c([2H])c([2H])c(-c2cc(-c3ccc(N(c4ccc5c(c4)C(C)(C)c4ccccc4-5)c4ccccc4-c4ccccc4)cc3)c(-c3c([2H])c([2H])c([2H])c([2H])c3[2H])c3c2oc2ccccc23)c([2H])c1[2H]]

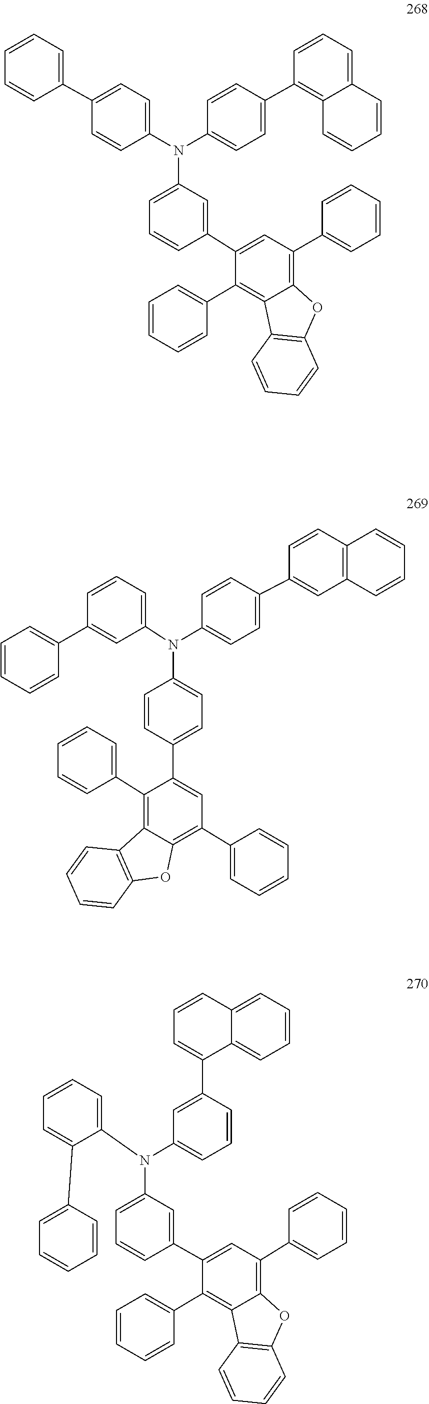 [SMILES: c1ccc(-c2ccc(N(c3ccc(-c4cccc5ccccc45)cc3)c3cccc(-c4cc(-c5ccccc5)c5oc6ccccc6c5c4-c4ccccc4)c3)cc2)cc1 ; c1ccc(-c2cccc(N(c3ccc(-c4ccc5ccccc5c4)cc3)c3ccc(-c4cc(-c5ccccc5)c5oc6ccccc6c5c4-c4ccccc4)cc3)c2)cc1 ; c1ccc(-c2ccccc2N(c2cccc(-c3cc(-c4ccccc4)c4oc5ccccc5c4c3-c3ccccc3)c2)c2cccc(-c3cccc4ccccc34)c2)cc1]

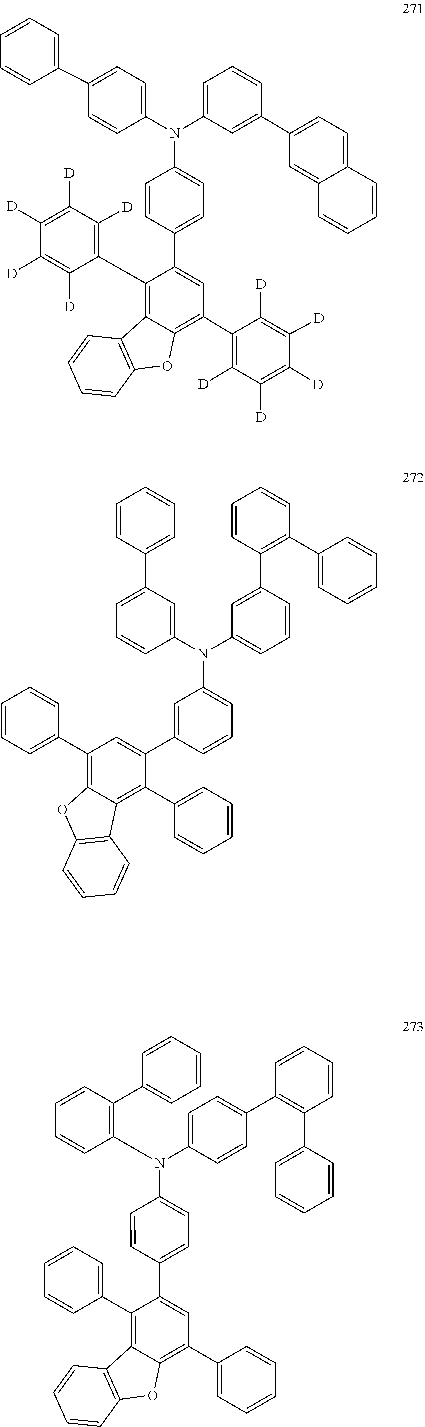 [SMILES: [2H]c1c([2H])c([2H])c(-c2cc(-c3ccc(N(c4ccc(-c5ccccc5)cc4)c4cccc(-c5ccc6ccccc6c5)c4)cc3)c(-c3c([2H])c([2H])c([2H])c([2H])c3[2H])c3c2oc2ccccc23)c([2H])c1[2H] ; c1ccc(-c2cccc(N(c3cccc(-c4ccccc4-c4ccccc4)c3)c3cccc(-c4cc(-c5ccccc5)c5oc6ccccc6c5c4-c4ccccc4)c3)c2)cc1 ; c1ccc(-c2ccccc2-c2ccc(N(c3ccc(-c4cc(-c5ccccc5)c5oc6ccccc6c5c4-c4ccccc4)cc3)c3ccccc3-c3ccccc3)cc2)cc1]

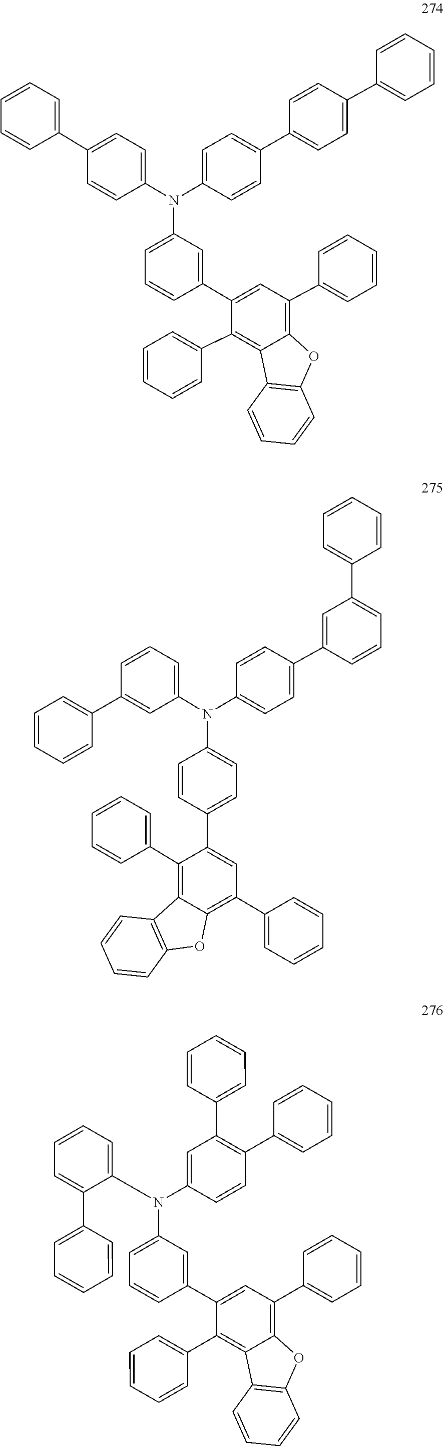 [SMILES: c1ccc(-c2ccc(-c3ccc(N(c4ccc(-c5ccccc5)cc4)c4cccc(-c5cc(-c6ccccc6)c6oc7ccccc7c6c5-c5ccccc5)c4)cc3)cc2)cc1 ; c1ccc(-c2cccc(-c3ccc(N(c4ccc(-c5cc(-c6ccccc6)c6oc7ccccc7c6c5-c5ccccc5)cc4)c4cccc(-c5ccccc5)c4)cc3)c2)cc1 ; c1ccc(-c2ccc(N(c3cccc(-c4cc(-c5ccccc5)c5oc6ccccc6c5c4-c4ccccc4)c3)c3ccccc3-c3ccccc3)cc2-c2ccccc2)cc1]

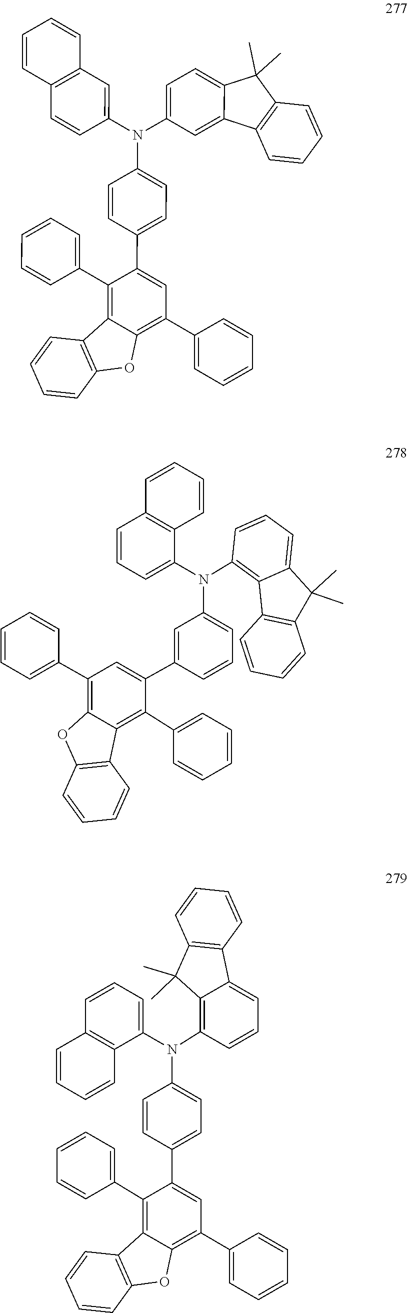 [SMILES: CC1(C)c2ccccc2-c2cc(N(c3ccc(-c4cc(-c5ccccc5)c5oc6ccccc6c5c4-c4ccccc4)cc3)c3ccc4ccccc4c3)ccc21 ; CC1(C)c2ccccc2-c2c(N(c3cccc(-c4cc(-c5ccccc5)c5oc6ccccc6c5c4-c4ccccc4)c3)c3cccc4ccccc34)cccc21 ; CC1(C)c2ccccc2-c2cccc(N(c3ccc(-c4cc(-c5ccccc5)c5oc6ccccc6c5c4-c4ccccc4)cc3)c3cccc4ccccc34)c21]

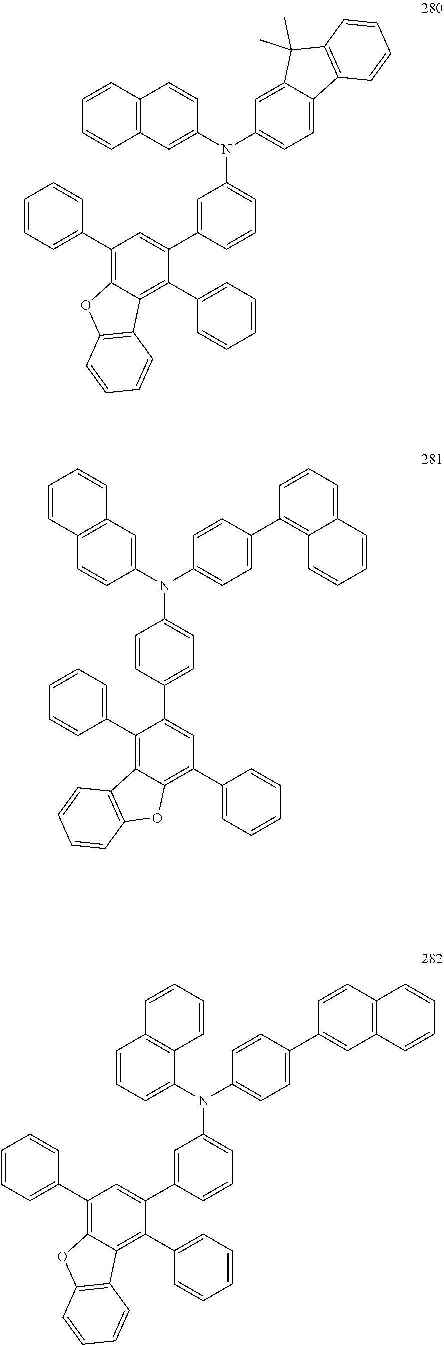 [SMILES: CC1(C)c2ccccc2-c2ccc(N(c3cccc(-c4cc(-c5ccccc5)c5oc6ccccc6c5c4-c4ccccc4)c3)c3ccc4ccccc4c3)cc21 ; c1ccc(-c2cc(-c3ccc(N(c4ccc(-c5cccc6ccccc56)cc4)c4ccc5ccccc5c4)cc3)c(-c3ccccc3)c3c2oc2ccccc23)cc1 ; c1ccc(-c2cc(-c3cccc(N(c4ccc(-c5ccc6ccccc6c5)cc4)c4cccc5ccccc45)c3)c(-c3ccccc3)c3c2oc2ccccc23)cc1]

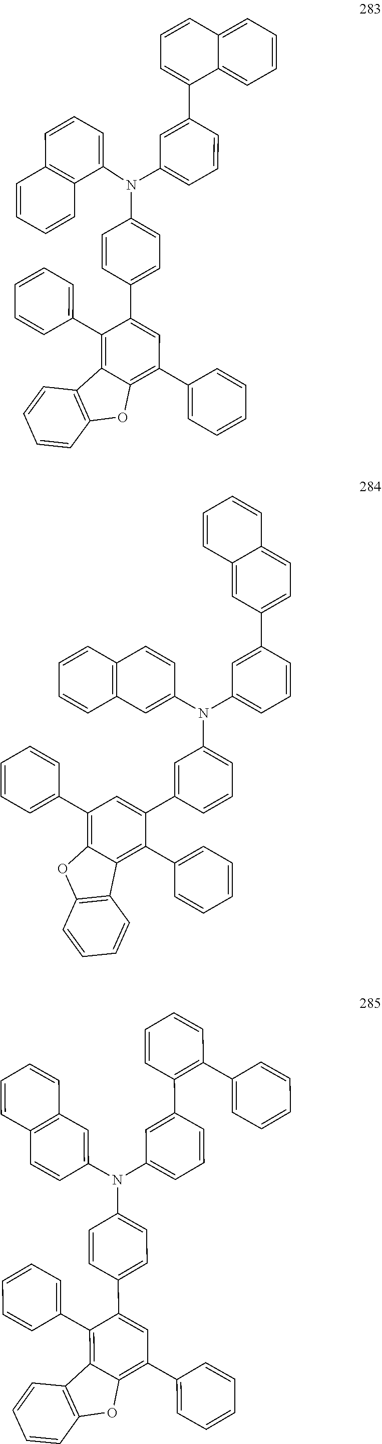 [SMILES: c1ccc(-c2cc(-c3ccc(N(c4cccc(-c5cccc6ccccc56)c4)c4cccc5ccccc45)cc3)c(-c3ccccc3)c3c2oc2ccccc23)cc1 ; c1ccc(-c2cc(-c3cccc(N(c4cccc(-c5ccc6ccccc6c5)c4)c4ccc5ccccc5c4)c3)c(-c3ccccc3)c3c2oc2ccccc23)cc1 ; c1ccc(-c2ccccc2-c2cccc(N(c3ccc(-c4cc(-c5ccccc5)c5oc6ccccc6c5c4-c4ccccc4)cc3)c3ccc4ccccc4c3)c2)cc1]

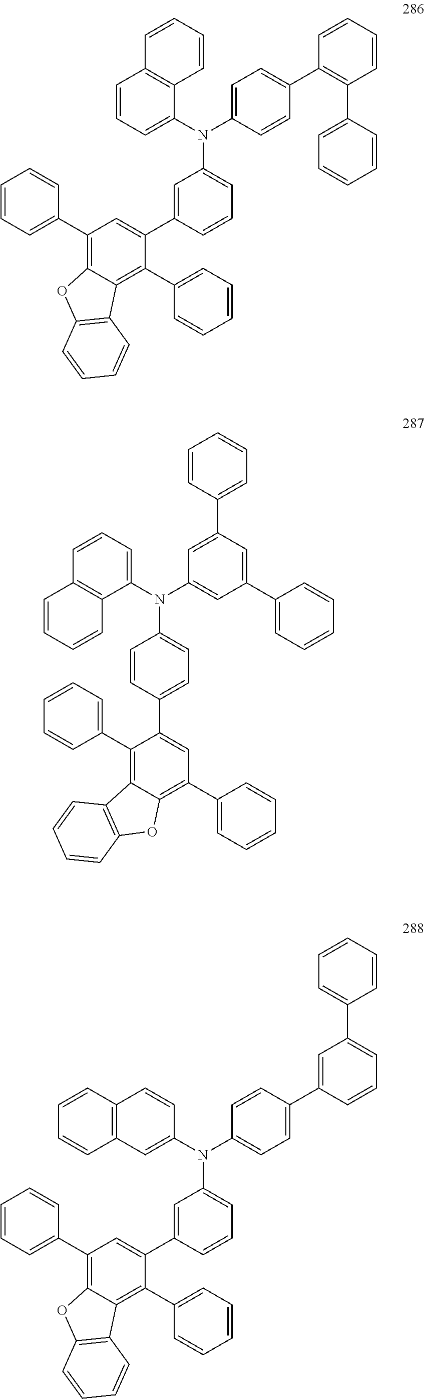 [SMILES: c1ccc(-c2ccccc2-c2ccc(N(c3cccc(-c4cc(-c5ccccc5)c5oc6ccccc6c5c4-c4ccccc4)c3)c3cccc4ccccc34)cc2)cc1 ; c1ccc(-c2cc(-c3ccccc3)cc(N(c3ccc(-c4cc(-c5ccccc5)c5oc6ccccc6c5c4-c4ccccc4)cc3)c3cccc4ccccc34)c2)cc1 ; c1ccc(-c2cccc(-c3ccc(N(c4cccc(-c5cc(-c6ccccc6)c6oc7ccccc7c6c5-c5ccccc5)c4)c4ccc5ccccc5c4)cc3)c2)cc1]

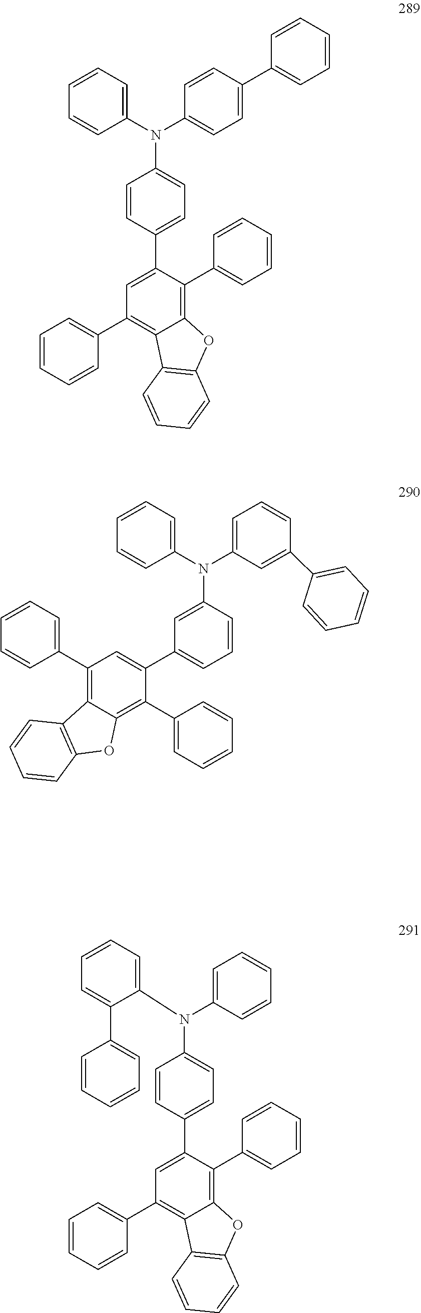 [SMILES: c1ccc(-c2ccc(N(c3ccccc3)c3ccc(-c4cc(-c5ccccc5)c5c(oc6ccccc65)c4-c4ccccc4)cc3)cc2)cc1 ; c1ccc(-c2cccc(N(c3ccccc3)c3cccc(-c4cc(-c5ccccc5)c5c(oc6ccccc65)c4-c4ccccc4)c3)c2)cc1 ; c1ccc(-c2ccccc2N(c2ccccc2)c2ccc(-c3cc(-c4ccccc4)c4c(oc5ccccc54)c3-c3ccccc3)cc2)cc1]

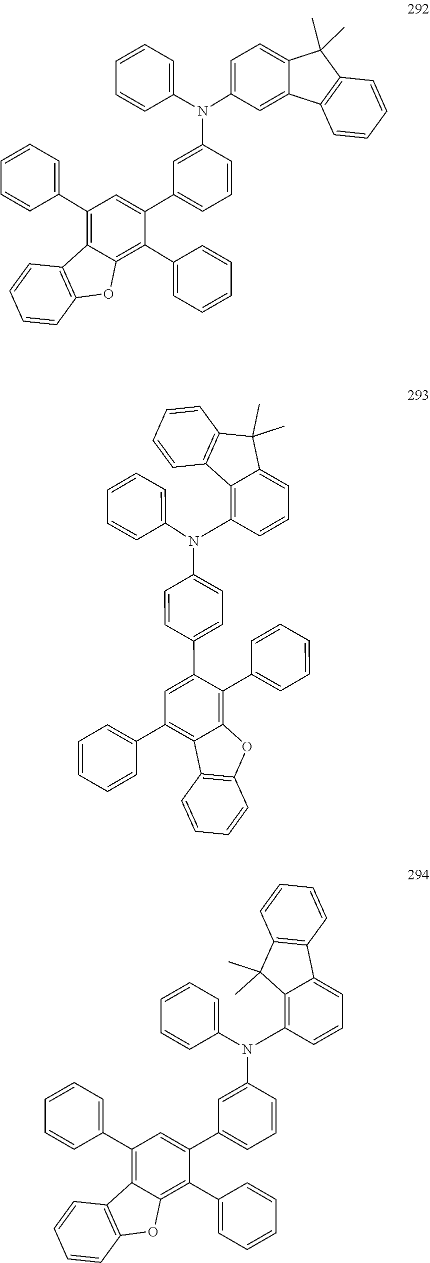 [SMILES: CC1(C)c2ccccc2-c2cc(N(c3ccccc3)c3cccc(-c4cc(-c5ccccc5)c5c(oc6ccccc65)c4-c4ccccc4)c3)ccc21 ; CC1(C)c2ccccc2-c2c(N(c3ccccc3)c3ccc(-c4cc(-c5ccccc5)c5c(oc6ccccc65)c4-c4ccccc4)cc3)cccc21 ; CC1(C)c2ccccc2-c2cccc(N(c3ccccc3)c3cccc(-c4cc(-c5ccccc5)c5c(oc6ccccc65)c4-c4ccccc4)c3)c21]

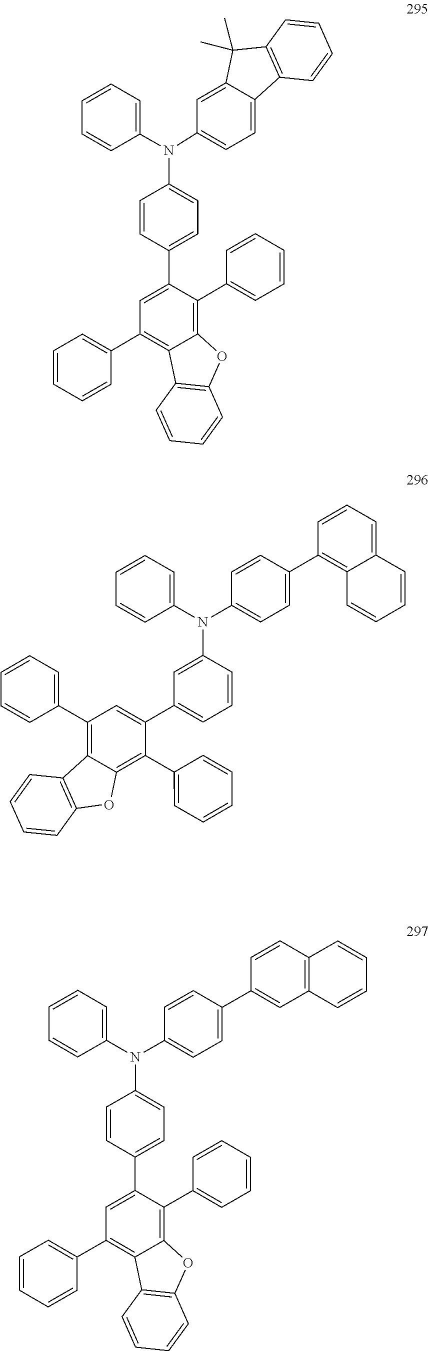 [SMILES: CC1(C)c2ccccc2-c2ccc(N(c3ccccc3)c3ccc(-c4cc(-c5ccccc5)c5c(oc6ccccc65)c4-c4ccccc4)cc3)cc21 ; c1ccc(-c2c(-c3cccc(N(c4ccccc4)c4ccc(-c5cccc6ccccc56)cc4)c3)cc(-c3ccccc3)c3c2oc2ccccc23)cc1 ; c1ccc(-c2c(-c3ccc(N(c4ccccc4)c4ccc(-c5ccc6ccccc6c5)cc4)cc3)cc(-c3ccccc3)c3c2oc2ccccc23)cc1]

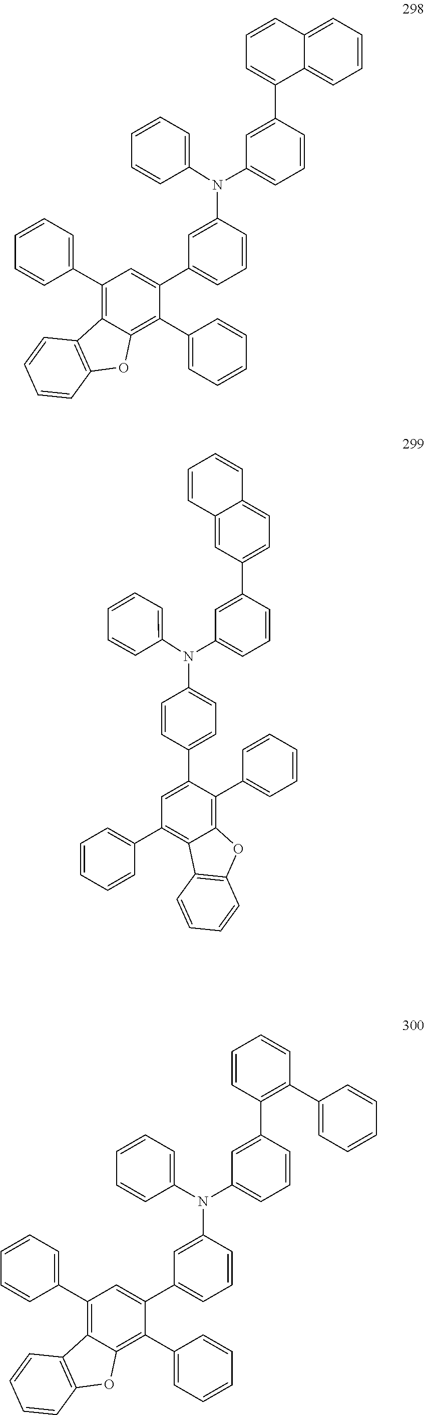 [SMILES: c1ccc(-c2c(-c3cccc(N(c4ccccc4)c4cccc(-c5cccc6ccccc56)c4)c3)cc(-c3ccccc3)c3c2oc2ccccc23)cc1 ; c1ccc(-c2c(-c3ccc(N(c4ccccc4)c4cccc(-c5ccc6ccccc6c5)c4)cc3)cc(-c3ccccc3)c3c2oc2ccccc23)cc1 ; c1ccc(-c2ccccc2-c2cccc(N(c3ccccc3)c3cccc(-c4cc(-c5ccccc5)c5c(oc6ccccc65)c4-c4ccccc4)c3)c2)cc1]

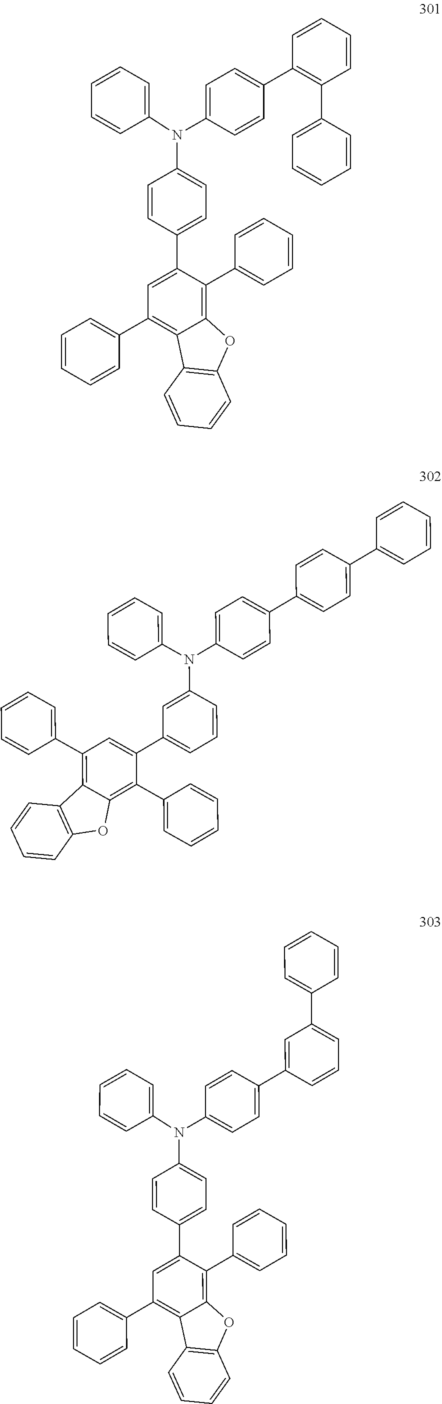 [SMILES: c1ccc(-c2ccccc2-c2ccc(N(c3ccccc3)c3ccc(-c4cc(-c5ccccc5)c5c(oc6ccccc65)c4-c4ccccc4)cc3)cc2)cc1 ; c1ccc(-c2ccc(-c3ccc(N(c4ccccc4)c4cccc(-c5cc(-c6ccccc6)c6c(oc7ccccc76)c5-c5ccccc5)c4)cc3)cc2)cc1 ; c1ccc(-c2cccc(-c3ccc(N(c4ccccc4)c4ccc(-c5cc(-c6ccccc6)c6c(oc7ccccc76)c5-c5ccccc5)cc4)cc3)c2)cc1]

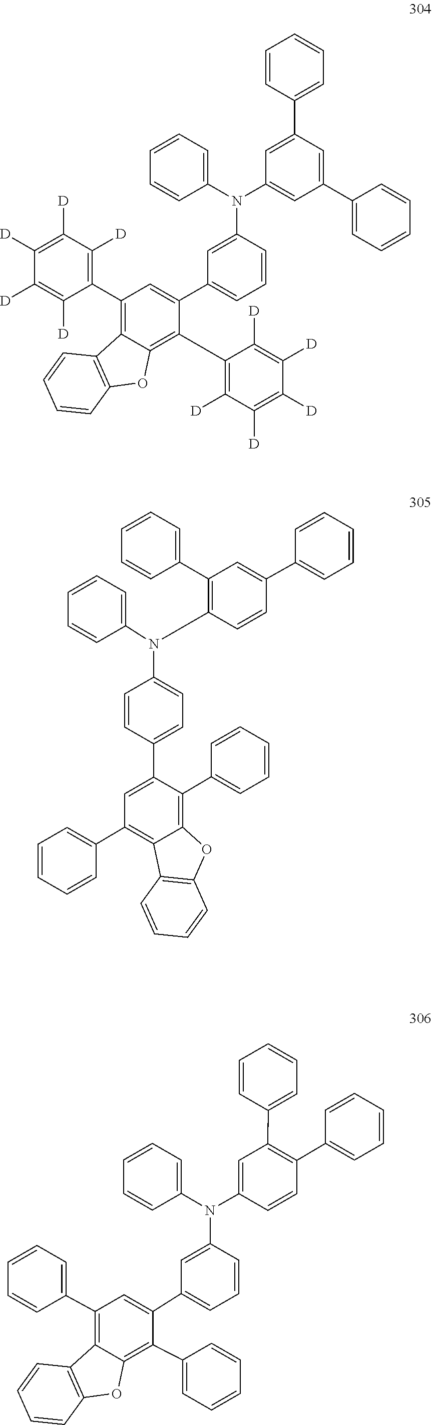 [SMILES: [2H]c1c([2H])c([2H])c(-c2c(-c3cccc(N(c4ccccc4)c4cc(-c5ccccc5)cc(-c5ccccc5)c4)c3)cc(-c3c([2H])c([2H])c([2H])c([2H])c3[2H])c3c2oc2ccccc23)c([2H])c1[2H] ; c1ccc(-c2ccc(N(c3ccccc3)c3ccc(-c4cc(-c5ccccc5)c5c(oc6ccccc65)c4-c4ccccc4)cc3)c(-c3ccccc3)c2)cc1 ; c1ccc(-c2ccc(N(c3ccccc3)c3cccc(-c4cc(-c5ccccc5)c5c(oc6ccccc65)c4-c4ccccc4)c3)cc2-c2ccccc2)cc1]

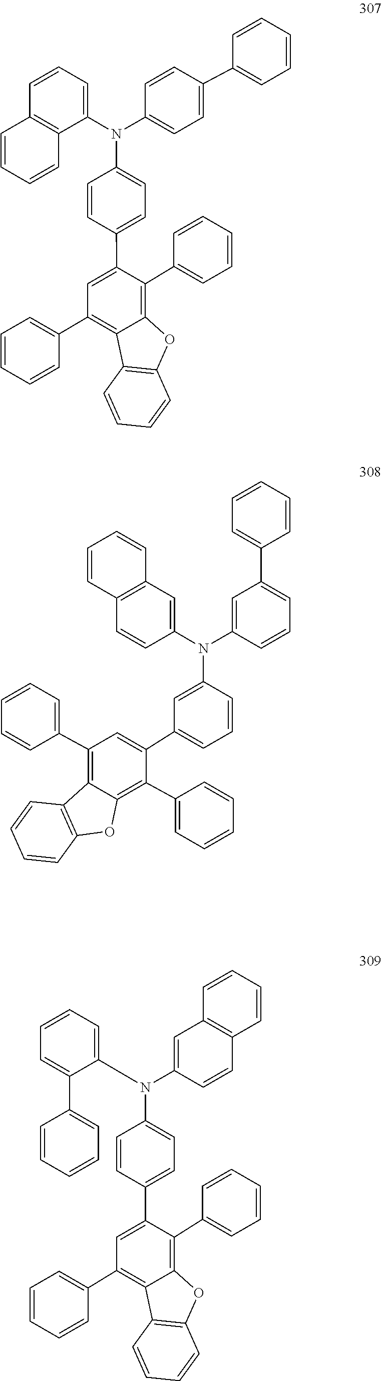 [SMILES: c1ccc(-c2ccc(N(c3ccc(-c4cc(-c5ccccc5)c5c(oc6ccccc65)c4-c4ccccc4)cc3)c3cccc4ccccc34)cc2)cc1 ; c1ccc(-c2cccc(N(c3cccc(-c4cc(-c5ccccc5)c5c(oc6ccccc65)c4-c4ccccc4)c3)c3ccc4ccccc4c3)c2)cc1 ; c1ccc(-c2ccccc2N(c2ccc(-c3cc(-c4ccccc4)c4c(oc5ccccc54)c3-c3ccccc3)cc2)c2ccc3ccccc3c2)cc1]

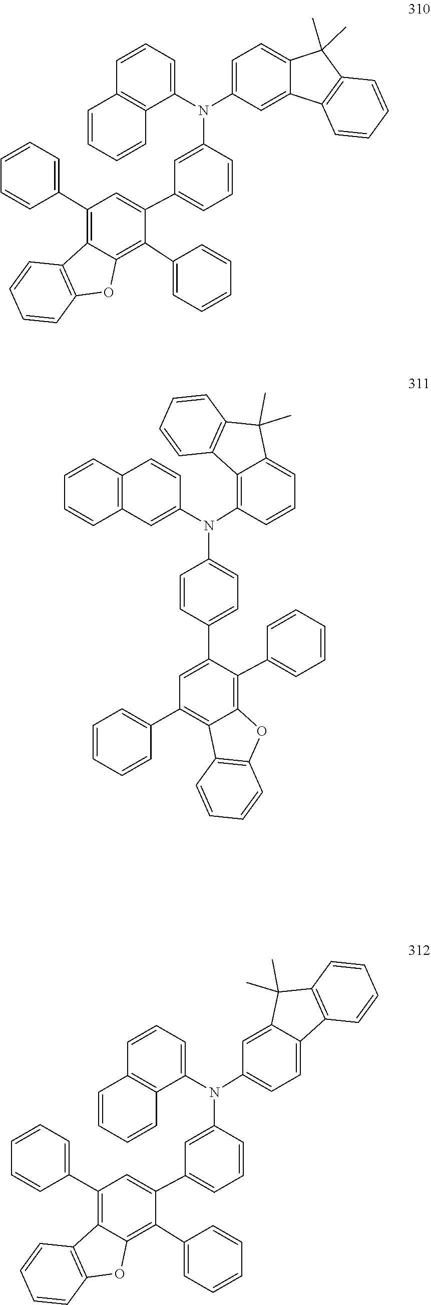 [SMILES: CC1(C)c2ccccc2-c2cc(N(c3cccc(-c4cc(-c5ccccc5)c5c(oc6ccccc65)c4-c4ccccc4)c3)c3cccc4ccccc34)ccc21 ; CC1(C)c2ccccc2-c2c(N(c3ccc(-c4cc(-c5ccccc5)c5c(oc6ccccc65)c4-c4ccccc4)cc3)c3ccc4ccccc4c3)cccc21 ; CC1(C)c2ccccc2-c2ccc(N(c3cccc(-c4cc(-c5ccccc5)c5c(oc6ccccc65)c4-c4ccccc4)c3)c3cccc4ccccc34)cc21]

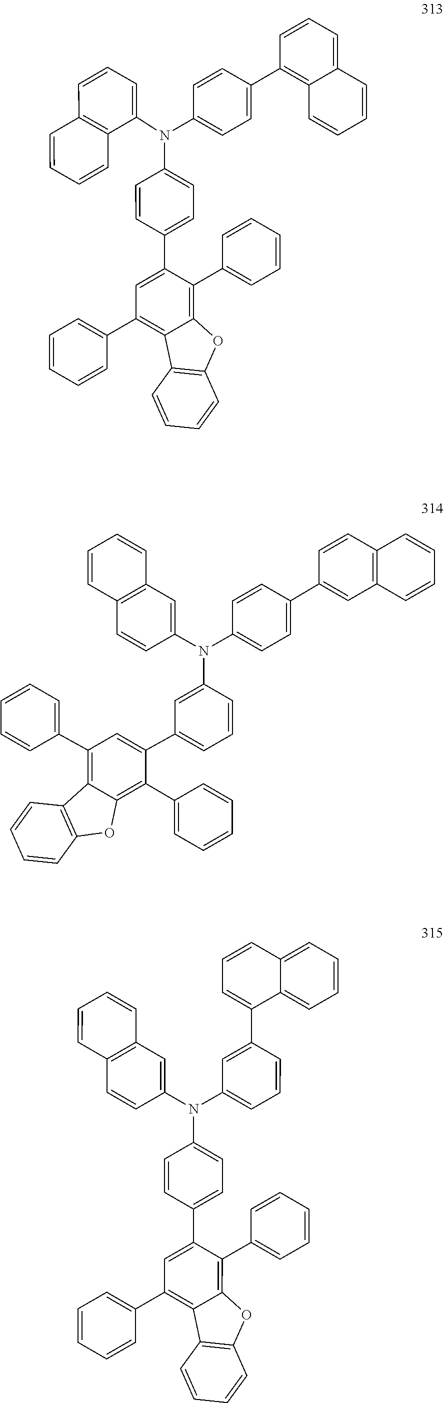 [SMILES: c1ccc(-c2c(-c3ccc(N(c4ccc(-c5cccc6ccccc56)cc4)c4cccc5ccccc45)cc3)cc(-c3ccccc3)c3c2oc2ccccc23)cc1 ; c1ccc(-c2c(-c3cccc(N(c4ccc(-c5ccc6ccccc6c5)cc4)c4ccc5ccccc5c4)c3)cc(-c3ccccc3)c3c2oc2ccccc23)cc1 ; c1ccc(-c2c(-c3ccc(N(c4cccc(-c5cccc6ccccc56)c4)c4ccc5ccccc5c4)cc3)cc(-c3ccccc3)c3c2oc2ccccc23)cc1]

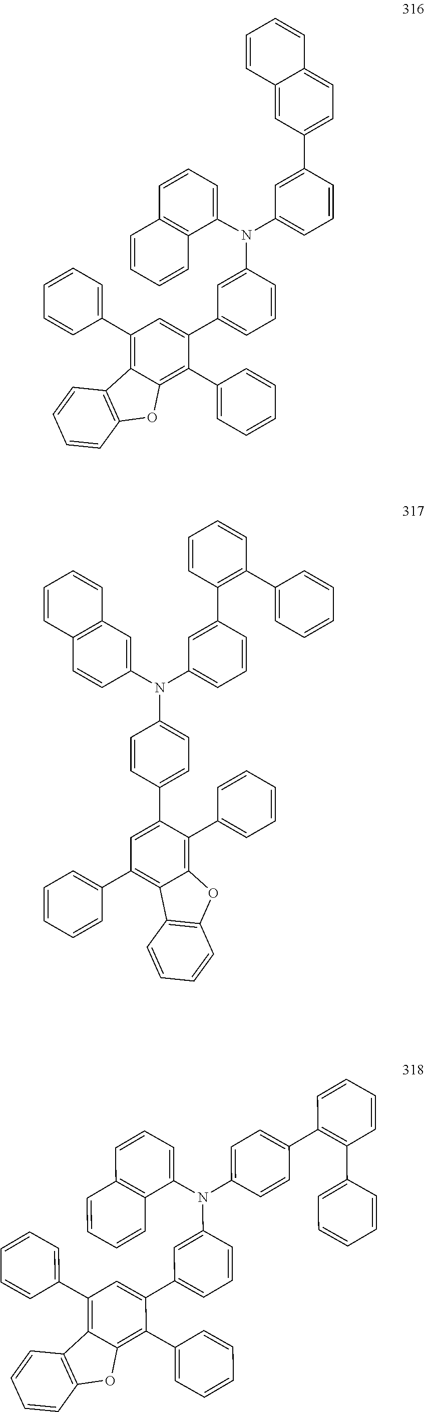 [SMILES: c1ccc(-c2c(-c3cccc(N(c4cccc(-c5ccc6ccccc6c5)c4)c4cccc5ccccc45)c3)cc(-c3ccccc3)c3c2oc2ccccc23)cc1 ; c1ccc(-c2ccccc2-c2cccc(N(c3ccc(-c4cc(-c5ccccc5)c5c(oc6ccccc65)c4-c4ccccc4)cc3)c3ccc4ccccc4c3)c2)cc1 ; c1ccc(-c2ccccc2-c2ccc(N(c3cccc(-c4cc(-c5ccccc5)c5c(oc6ccccc65)c4-c4ccccc4)c3)c3cccc4ccccc34)cc2)cc1]

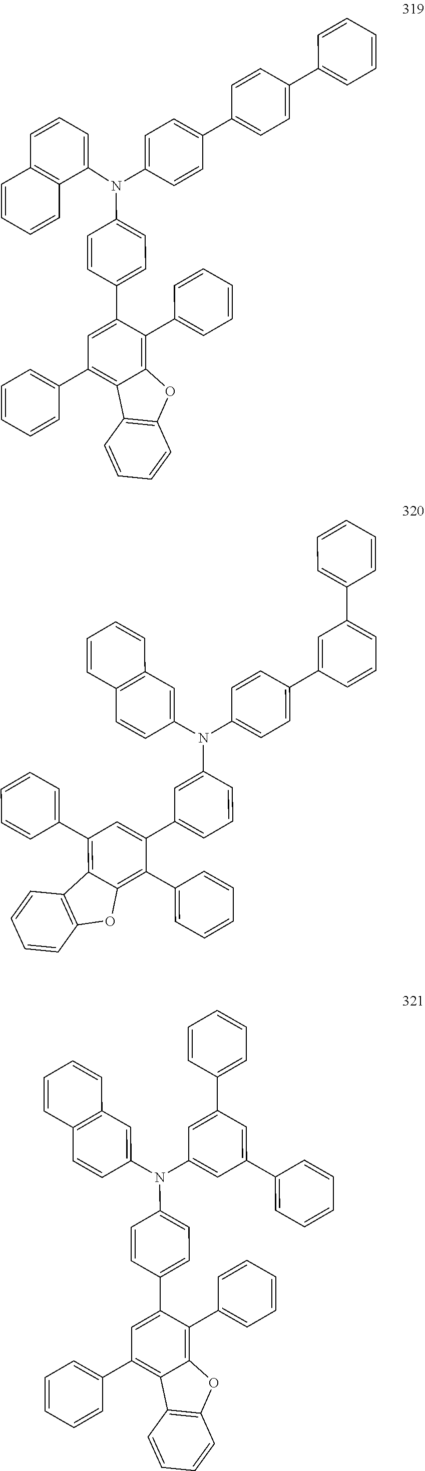 [SMILES: c1ccc(-c2ccc(-c3ccc(N(c4ccc(-c5cc(-c6ccccc6)c6c(oc7ccccc76)c5-c5ccccc5)cc4)c4cccc5ccccc45)cc3)cc2)cc1 ; c1ccc(-c2cccc(-c3ccc(N(c4cccc(-c5cc(-c6ccccc6)c6c(oc7ccccc76)c5-c5ccccc5)c4)c4ccc5ccccc5c4)cc3)c2)cc1 ; c1ccc(-c2cc(-c3ccccc3)cc(N(c3ccc(-c4cc(-c5ccccc5)c5c(oc6ccccc65)c4-c4ccccc4)cc3)c3ccc4ccccc4c3)c2)cc1]

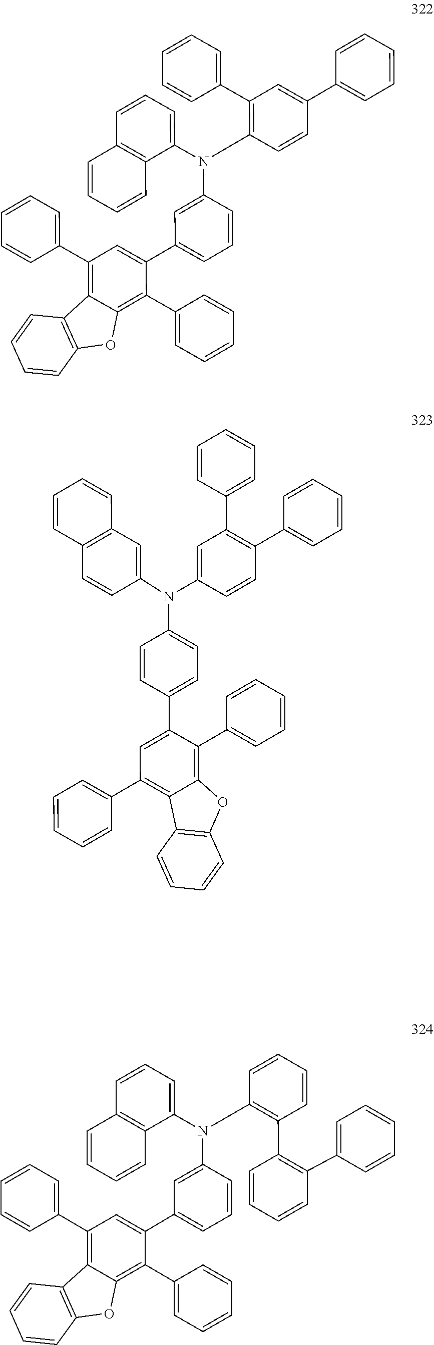 [SMILES: c1ccc(-c2ccc(N(c3cccc(-c4cc(-c5ccccc5)c5c(oc6ccccc65)c4-c4ccccc4)c3)c3cccc4ccccc34)c(-c3ccccc3)c2)cc1 ; c1ccc(-c2ccc(N(c3ccc(-c4cc(-c5ccccc5)c5c(oc6ccccc65)c4-c4ccccc4)cc3)c3ccc4ccccc4c3)cc2-c2ccccc2)cc1 ; c1ccc(-c2ccccc2-c2ccccc2N(c2cccc(-c3cc(-c4ccccc4)c4c(oc5ccccc54)c3-c3ccccc3)c2)c2cccc3ccccc23)cc1]

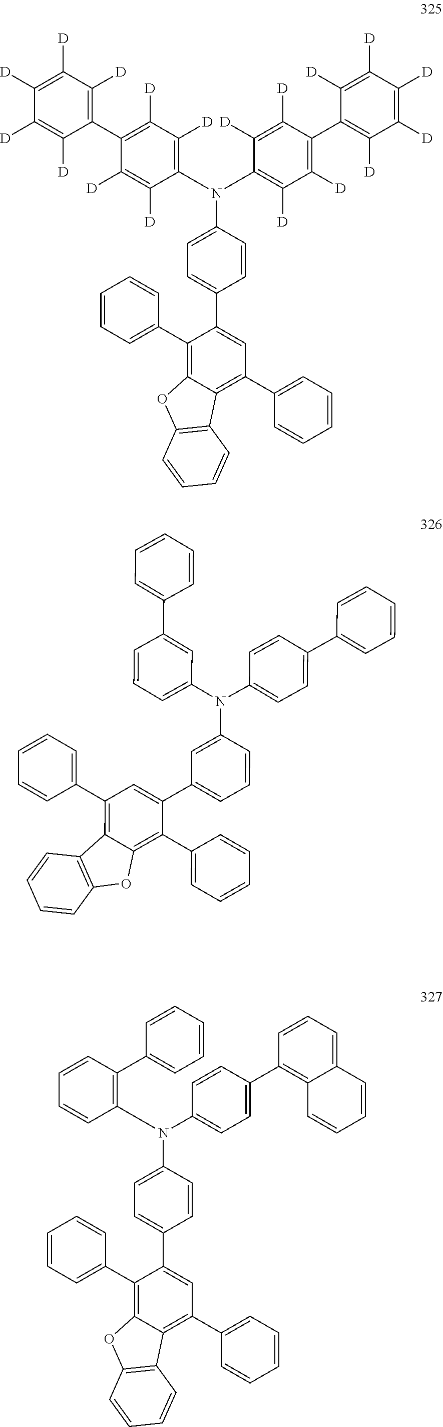 [SMILES: [2H]c1c([2H])c([2H])c(-c2c([2H])c([2H])c(N(c3ccc(-c4cc(-c5ccccc5)c5c(oc6ccccc65)c4-c4ccccc4)cc3)c3c([2H])c([2H])c(-c4c([2H])c([2H])c([2H])c([2H])c4[2H])c([2H])c3[2H])c([2H])c2[2H])c([2H])c1[2H] ; c1ccc(-c2ccc(N(c3cccc(-c4ccccc4)c3)c3cccc(-c4cc(-c5ccccc5)c5c(oc6ccccc65)c4-c4ccccc4)c3)cc2)cc1 ; c1ccc(-c2ccccc2N(c2ccc(-c3cc(-c4ccccc4)c4c(oc5ccccc54)c3-c3ccccc3)cc2)c2ccc(-c3cccc4ccccc34)cc2)cc1]

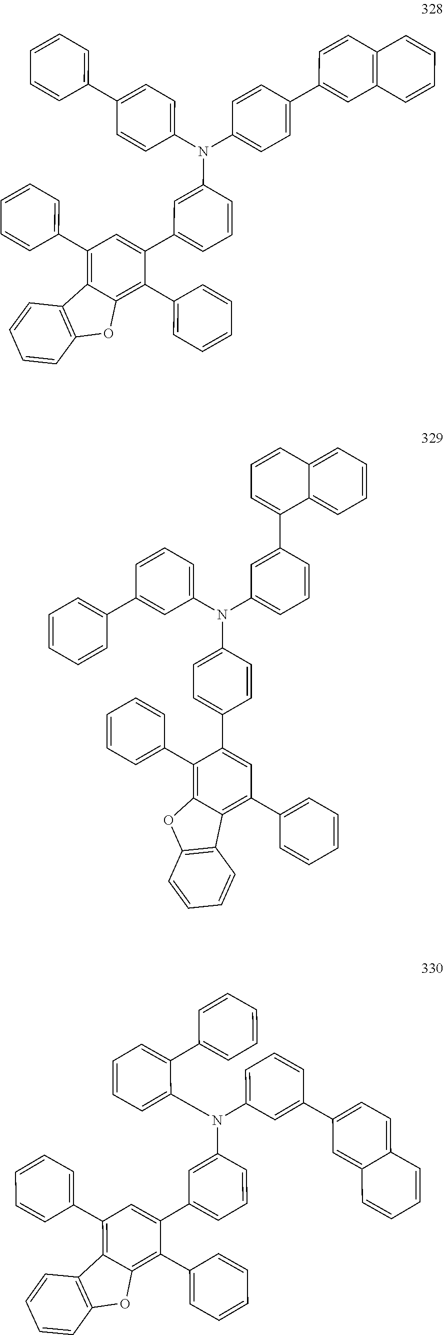 [SMILES: c1ccc(-c2ccc(N(c3ccc(-c4ccc5ccccc5c4)cc3)c3cccc(-c4cc(-c5ccccc5)c5c(oc6ccccc65)c4-c4ccccc4)c3)cc2)cc1 ; c1ccc(-c2cccc(N(c3ccc(-c4cc(-c5ccccc5)c5c(oc6ccccc65)c4-c4ccccc4)cc3)c3cccc(-c4cccc5ccccc45)c3)c2)cc1 ; c1ccc(-c2ccccc2N(c2cccc(-c3ccc4ccccc4c3)c2)c2cccc(-c3cc(-c4ccccc4)c4c(oc5ccccc54)c3-c3ccccc3)c2)cc1]

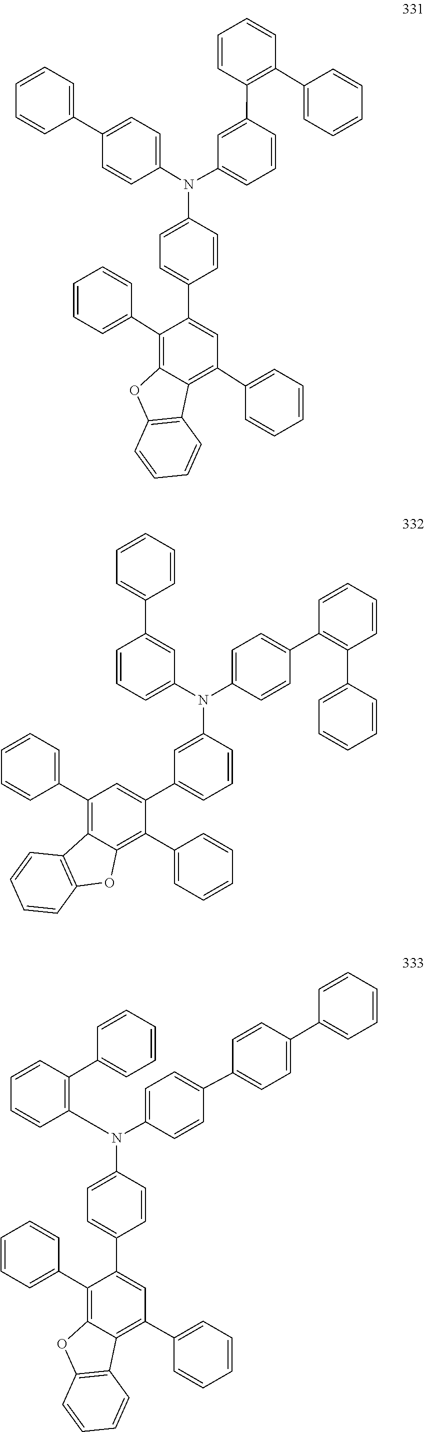 [SMILES: c1ccc(-c2ccc(N(c3ccc(-c4cc(-c5ccccc5)c5c(oc6ccccc65)c4-c4ccccc4)cc3)c3cccc(-c4ccccc4-c4ccccc4)c3)cc2)cc1 ; c1ccc(-c2cccc(N(c3ccc(-c4ccccc4-c4ccccc4)cc3)c3cccc(-c4cc(-c5ccccc5)c5c(oc6ccccc65)c4-c4ccccc4)c3)c2)cc1 ; c1ccc(-c2ccc(-c3ccc(N(c4ccc(-c5cc(-c6ccccc6)c6c(oc7ccccc76)c5-c5ccccc5)cc4)c4ccccc4-c4ccccc4)cc3)cc2)cc1]

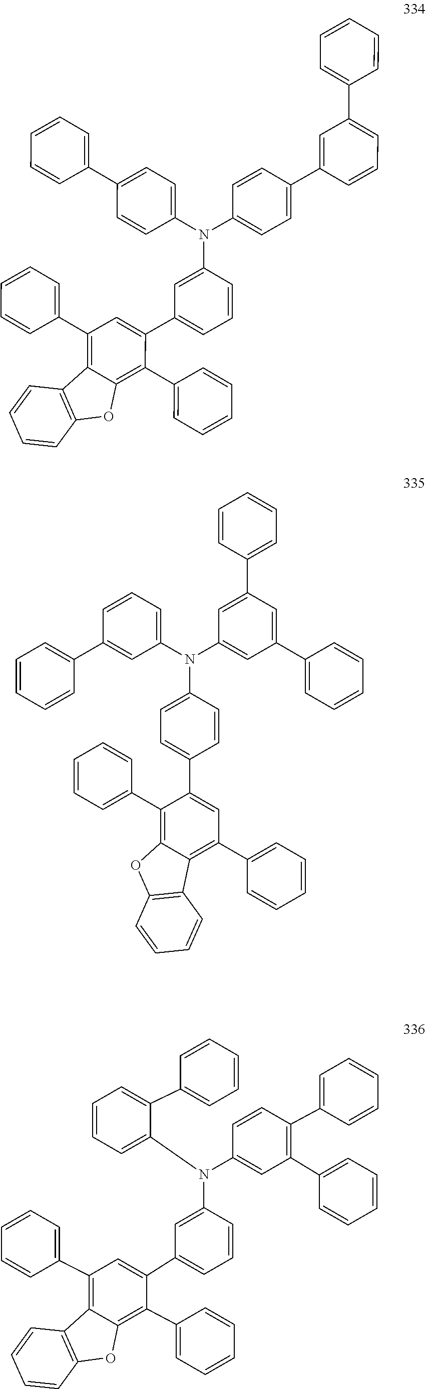 [SMILES: c1ccc(-c2ccc(N(c3ccc(-c4cccc(-c5ccccc5)c4)cc3)c3cccc(-c4cc(-c5ccccc5)c5c(oc6ccccc65)c4-c4ccccc4)c3)cc2)cc1 ; c1ccc(-c2cccc(N(c3ccc(-c4cc(-c5ccccc5)c5c(oc6ccccc65)c4-c4ccccc4)cc3)c3cc(-c4ccccc4)cc(-c4ccccc4)c3)c2)cc1 ; c1ccc(-c2ccc(N(c3cccc(-c4cc(-c5ccccc5)c5c(oc6ccccc65)c4-c4ccccc4)c3)c3ccccc3-c3ccccc3)cc2-c2ccccc2)cc1]

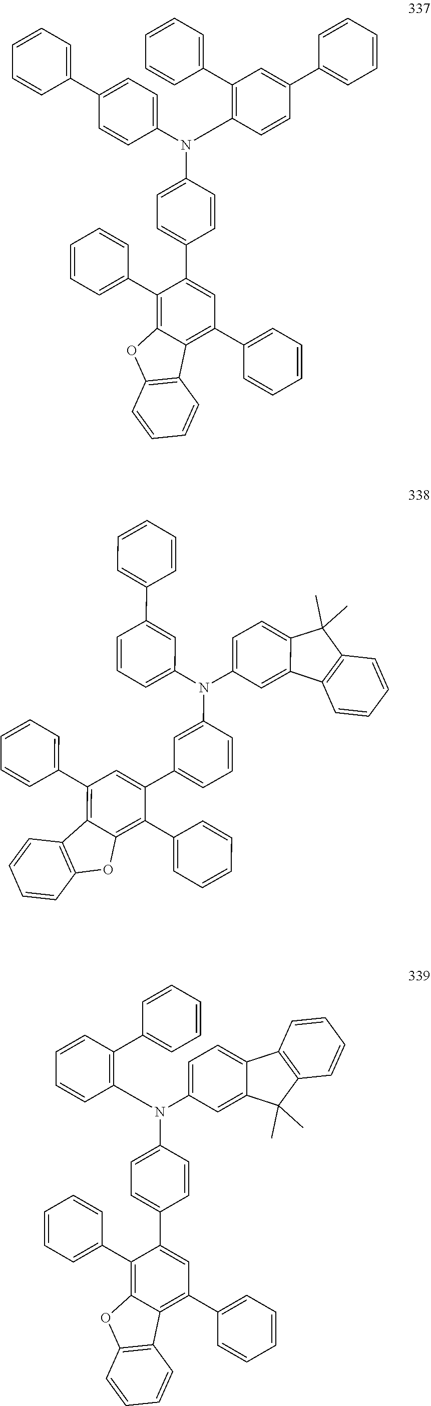 [SMILES: c1ccc(-c2ccc(N(c3ccc(-c4cc(-c5ccccc5)c5c(oc6ccccc65)c4-c4ccccc4)cc3)c3ccc(-c4ccccc4)cc3-c3ccccc3)cc2)cc1 ; CC1(C)c2ccccc2-c2cc(N(c3cccc(-c4ccccc4)c3)c3cccc(-c4cc(-c5ccccc5)c5c(oc6ccccc65)c4-c4ccccc4)c3)ccc21 ; CC1(C)c2ccccc2-c2ccc(N(c3ccc(-c4cc(-c5ccccc5)c5c(oc6ccccc65)c4-c4ccccc4)cc3)c3ccccc3-c3ccccc3)cc21]

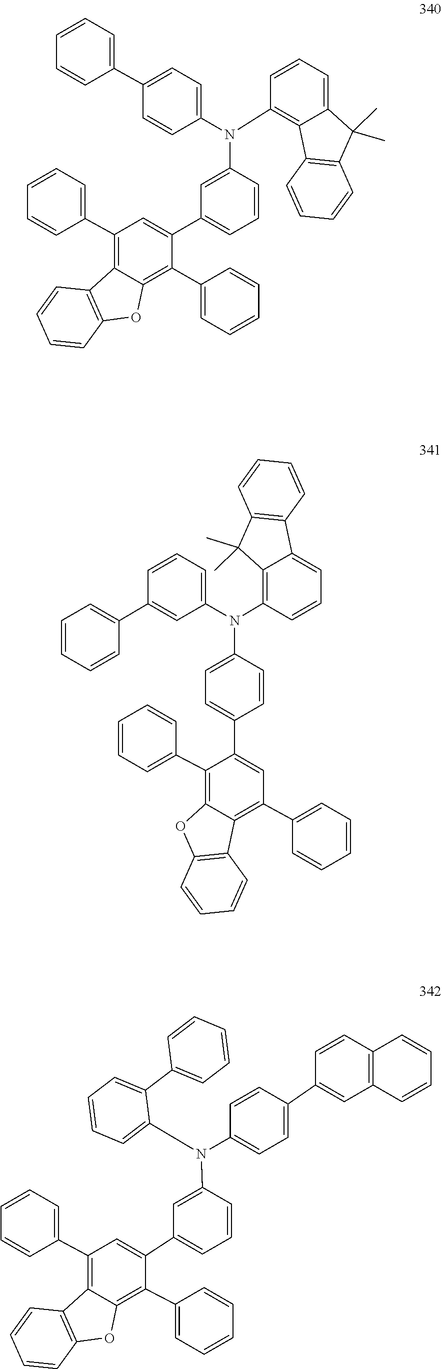 [SMILES: CC1(C)c2ccccc2-c2c(N(c3ccc(-c4ccccc4)cc3)c3cccc(-c4cc(-c5ccccc5)c5c(oc6ccccc65)c4-c4ccccc4)c3)cccc21 ; CC1(C)c2ccccc2-c2cccc(N(c3ccc(-c4cc(-c5ccccc5)c5c(oc6ccccc65)c4-c4ccccc4)cc3)c3cccc(-c4ccccc4)c3)c21 ; c1ccc(-c2ccccc2N(c2ccc(-c3ccc4ccccc4c3)cc2)c2cccc(-c3cc(-c4ccccc4)c4c(oc5ccccc54)c3-c3ccccc3)c2)cc1]

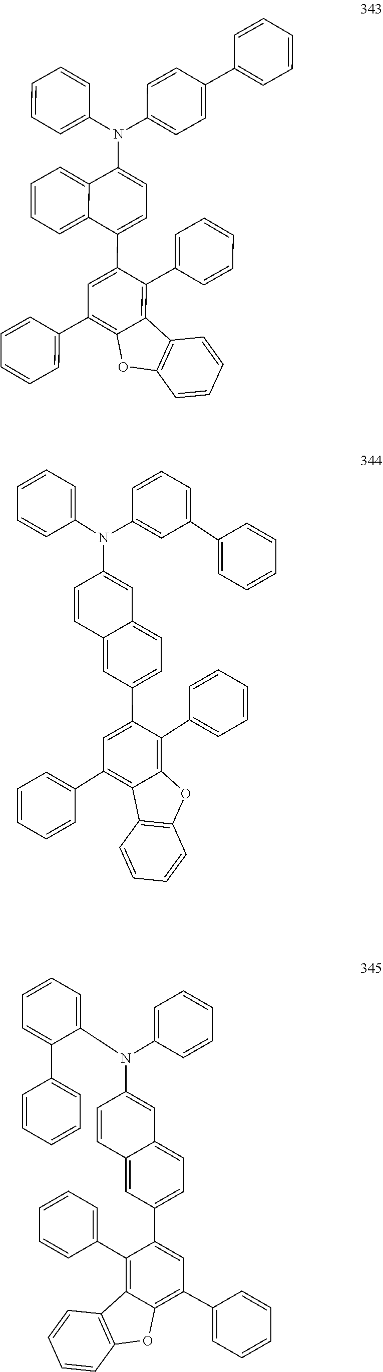 [SMILES: c1ccc(-c2ccc(N(c3ccccc3)c3ccc(-c4cc(-c5ccccc5)c5oc6ccccc6c5c4-c4ccccc4)c4ccccc34)cc2)cc1 ; c1ccc(-c2cccc(N(c3ccccc3)c3ccc4cc(-c5cc(-c6ccccc6)c6c(oc7ccccc76)c5-c5ccccc5)ccc4c3)c2)cc1 ; c1ccc(-c2ccccc2N(c2ccccc2)c2ccc3cc(-c4cc(-c5ccccc5)c5oc6ccccc6c5c4-c4ccccc4)ccc3c2)cc1]

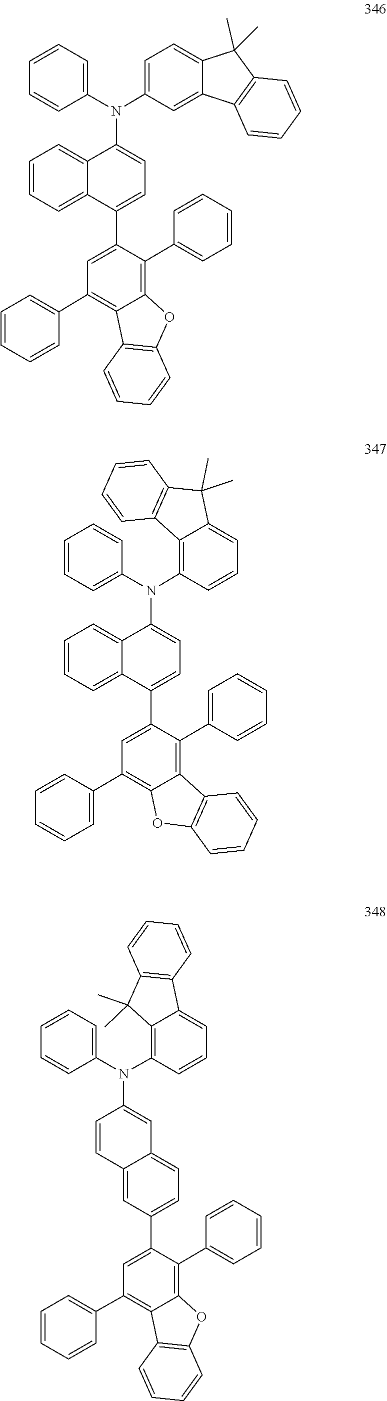 [SMILES: CC1(C)c2ccccc2-c2cc(N(c3ccccc3)c3ccc(-c4cc(-c5ccccc5)c5c(oc6ccccc65)c4-c4ccccc4)c4ccccc34)ccc21 ; CC1(C)c2ccccc2-c2c(N(c3ccccc3)c3ccc(-c4cc(-c5ccccc5)c5oc6ccccc6c5c4-c4ccccc4)c4ccccc34)cccc21 ; CC1(C)c2ccccc2-c2cccc(N(c3ccccc3)c3ccc4cc(-c5cc(-c6ccccc6)c6c(oc7ccccc76)c5-c5ccccc5)ccc4c3)c21]

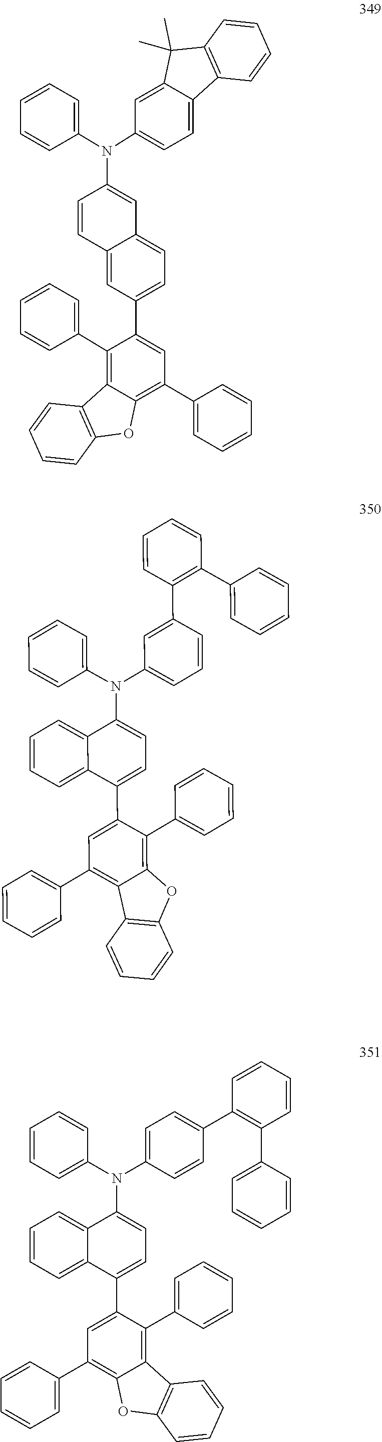 [SMILES: CC1(C)c2ccccc2-c2ccc(N(c3ccccc3)c3ccc4cc(-c5cc(-c6ccccc6)c6oc7ccccc7c6c5-c5ccccc5)ccc4c3)cc21 ; c1ccc(-c2ccccc2-c2cccc(N(c3ccccc3)c3ccc(-c4cc(-c5ccccc5)c5c(oc6ccccc65)c4-c4ccccc4)c4ccccc34)c2)cc1 ; c1ccc(-c2ccccc2-c2ccc(N(c3ccccc3)c3ccc(-c4cc(-c5ccccc5)c5oc6ccccc6c5c4-c4ccccc4)c4ccccc34)cc2)cc1]

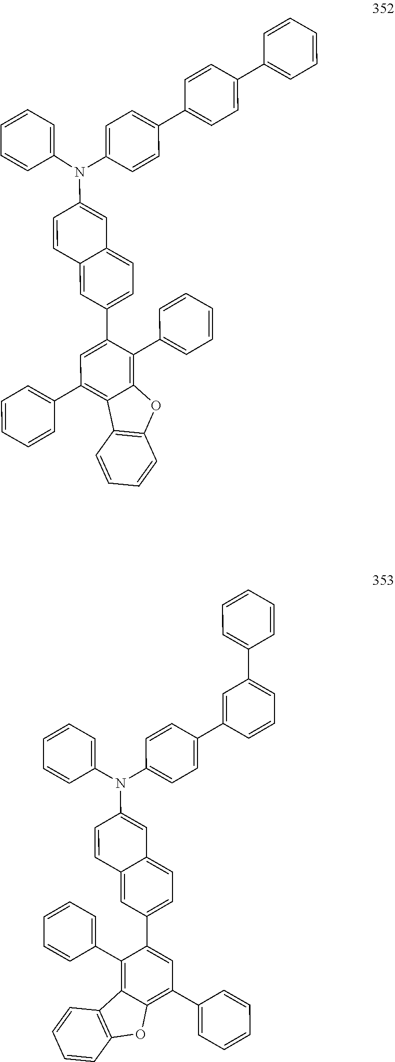 [SMILES: c1ccc(-c2ccc(-c3ccc(N(c4ccccc4)c4ccc5cc(-c6cc(-c7ccccc7)c7c(oc8ccccc87)c6-c6ccccc6)ccc5c4)cc3)cc2)cc1 ; c1ccc(-c2cccc(-c3ccc(N(c4ccccc4)c4ccc5cc(-c6cc(-c7ccccc7)c7oc8ccccc8c7c6-c6ccccc6)ccc5c4)cc3)c2)cc1]

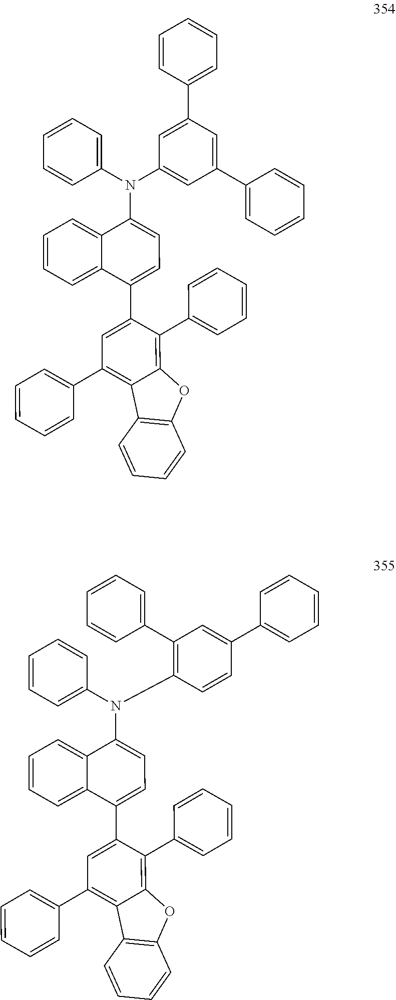 [SMILES: c1ccc(-c2cc(-c3ccccc3)cc(N(c3ccccc3)c3ccc(-c4cc(-c5ccccc5)c5c(oc6ccccc65)c4-c4ccccc4)c4ccccc34)c2)cc1 ; c1ccc(-c2ccc(N(c3ccccc3)c3ccc(-c4cc(-c5ccccc5)c5c(oc6ccccc65)c4-c4ccccc4)c4ccccc34)c(-c3ccccc3)c2)cc1]

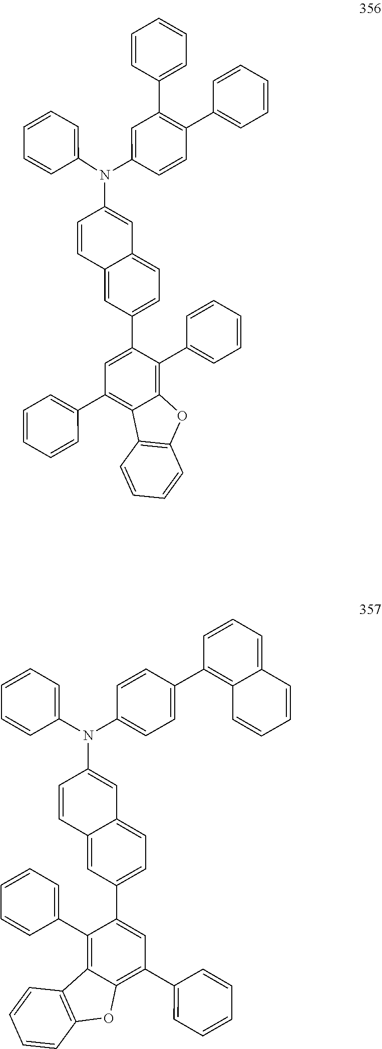 [SMILES: c1ccc(-c2ccc(N(c3ccccc3)c3ccc4cc(-c5cc(-c6ccccc6)c6c(oc7ccccc76)c5-c5ccccc5)ccc4c3)cc2-c2ccccc2)cc1 ; c1ccc(-c2cc(-c3ccc4cc(N(c5ccccc5)c5ccc(-c6cccc7ccccc67)cc5)ccc4c3)c(-c3ccccc3)c3c2oc2ccccc23)cc1]

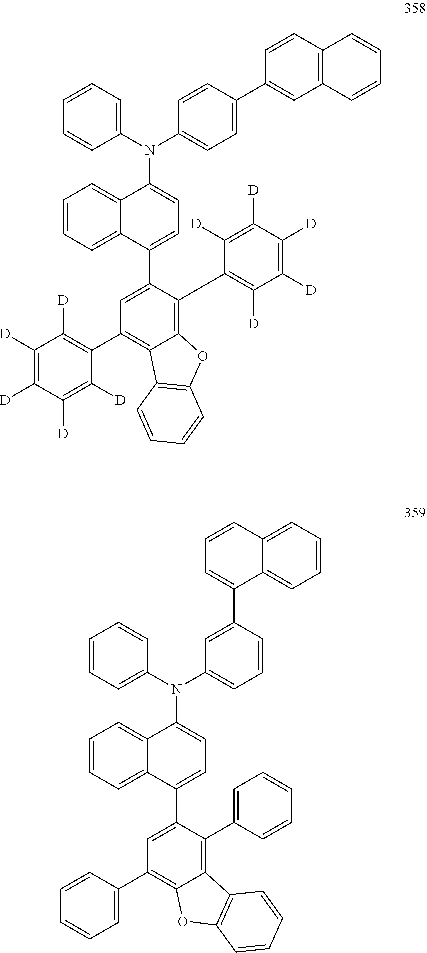 [SMILES: [2H]c1c([2H])c([2H])c(-c2c(-c3ccc(N(c4ccccc4)c4ccc(-c5ccc6ccccc6c5)cc4)c4ccccc34)cc(-c3c([2H])c([2H])c([2H])c([2H])c3[2H])c3c2oc2ccccc23)c([2H])c1[2H] ; c1ccc(-c2cc(-c3ccc(N(c4ccccc4)c4cccc(-c5cccc6ccccc56)c4)c4ccccc34)c(-c3ccccc3)c3c2oc2ccccc23)cc1]

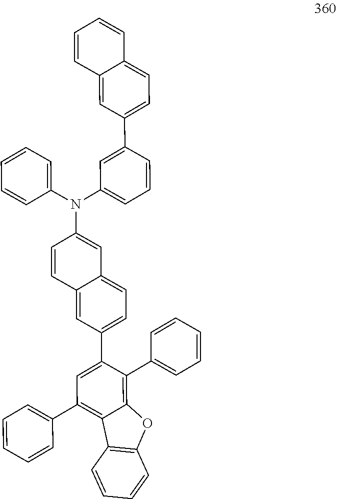 [SMILES: c1ccc(-c2c(-c3ccc4cc(N(c5ccccc5)c5cccc(-c6ccc7ccccc7c6)c5)ccc4c3)cc(-c3ccccc3)c3c2oc2ccccc23)cc1]

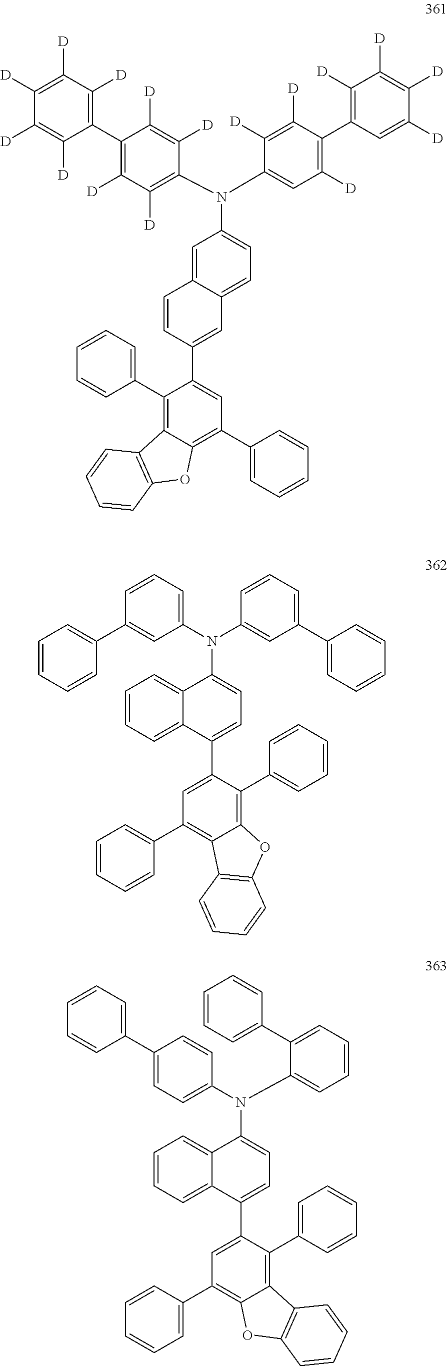

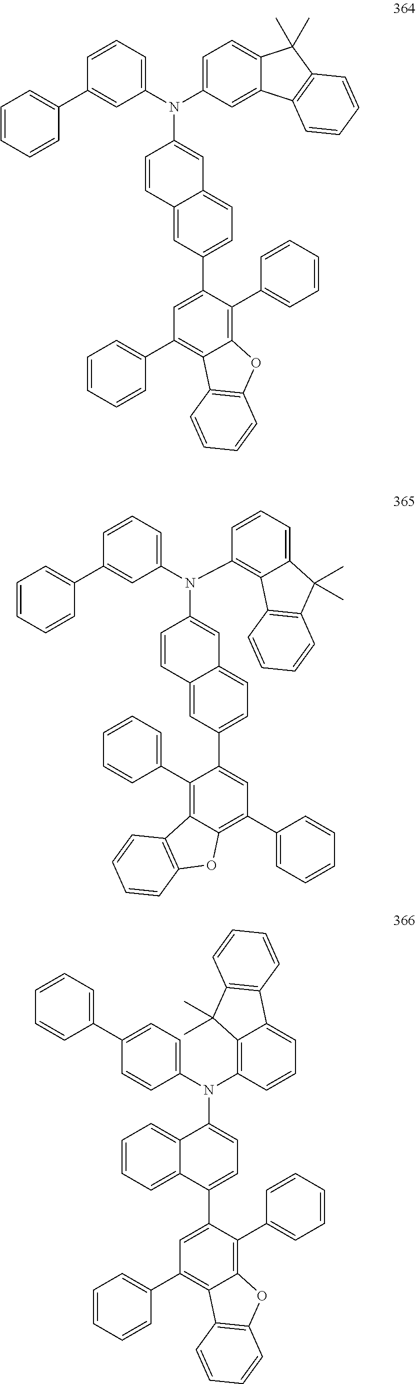 [SMILES: CC1(C)c2ccccc2-c2cc(N(c3cccc(-c4ccccc4)c3)c3ccc4cc(-c5cc(-c6ccccc6)c6c(oc7ccccc76)c5-c5ccccc5)ccc4c3)ccc21 ; CC1(C)c2ccccc2-c2c(N(c3cccc(-c4ccccc4)c3)c3ccc4cc(-c5cc(-c6ccccc6)c6oc7ccccc7c6c5-c5ccccc5)ccc4c3)cccc21 ; CC1(C)c2ccccc2-c2cccc(N(c3ccc(-c4ccccc4)cc3)c3ccc(-c4cc(-c5ccccc5)c5c(oc6ccccc65)c4-c4ccccc4)c4ccccc34)c21]

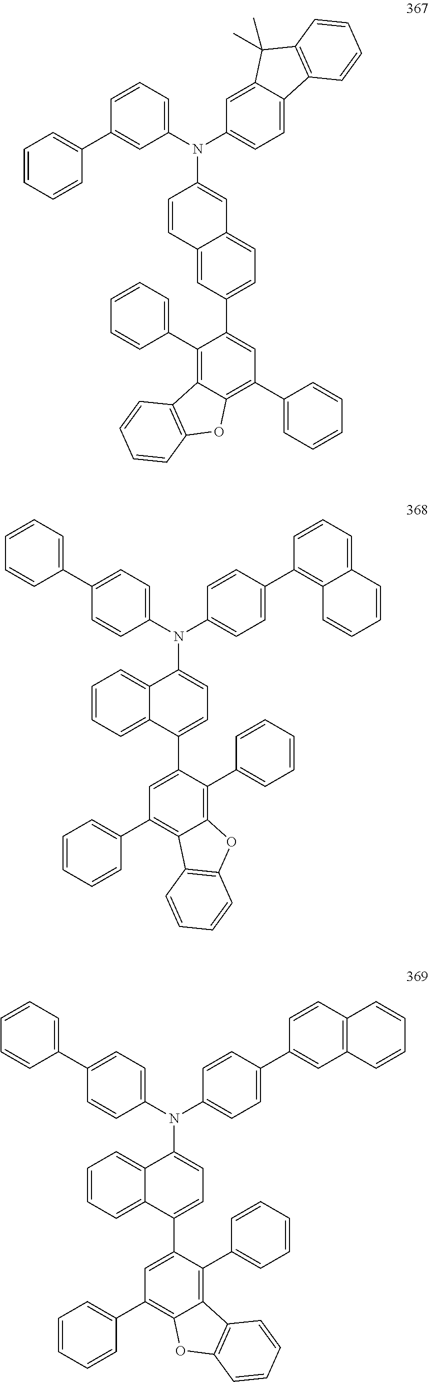 [SMILES: CC1(C)c2ccccc2-c2ccc(N(c3cccc(-c4ccccc4)c3)c3ccc4cc(-c5cc(-c6ccccc6)c6oc7ccccc7c6c5-c5ccccc5)ccc4c3)cc21 ; c1ccc(-c2ccc(N(c3ccc(-c4cccc5ccccc45)cc3)c3ccc(-c4cc(-c5ccccc5)c5c(oc6ccccc65)c4-c4ccccc4)c4ccccc34)cc2)cc1 ; c1ccc(-c2ccc(N(c3ccc(-c4ccc5ccccc5c4)cc3)c3ccc(-c4cc(-c5ccccc5)c5oc6ccccc6c5c4-c4ccccc4)c4ccccc34)cc2)cc1]

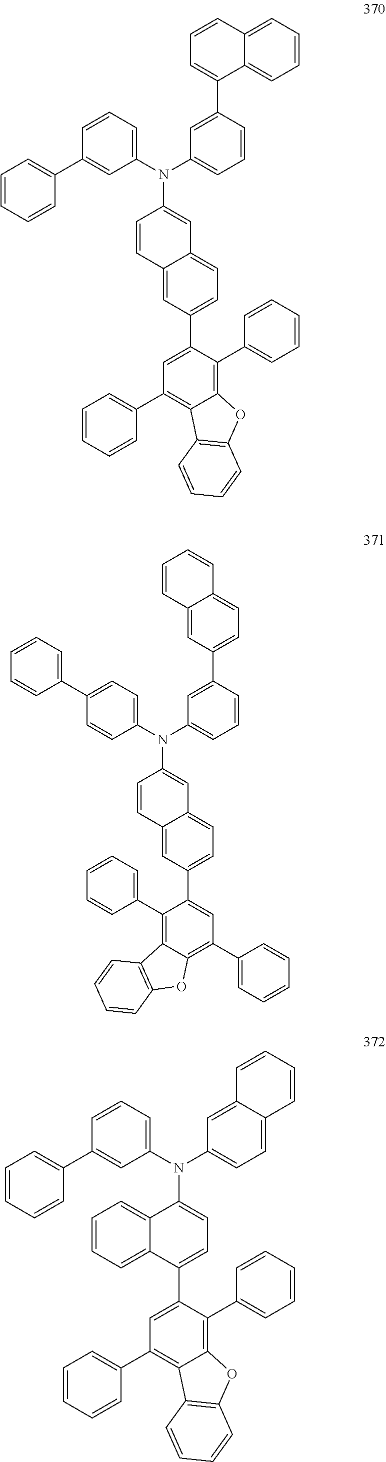 [SMILES: c1ccc(-c2cccc(N(c3cccc(-c4cccc5ccccc45)c3)c3ccc4cc(-c5cc(-c6ccccc6)c6c(oc7ccccc76)c5-c5ccccc5)ccc4c3)c2)cc1 ; c1ccc(-c2ccc(N(c3cccc(-c4ccc5ccccc5c4)c3)c3ccc4cc(-c5cc(-c6ccccc6)c6oc7ccccc7c6c5-c5ccccc5)ccc4c3)cc2)cc1 ; c1ccc(-c2cccc(N(c3ccc4ccccc4c3)c3ccc(-c4cc(-c5ccccc5)c5c(oc6ccccc65)c4-c4ccccc4)c4ccccc34)c2)cc1]

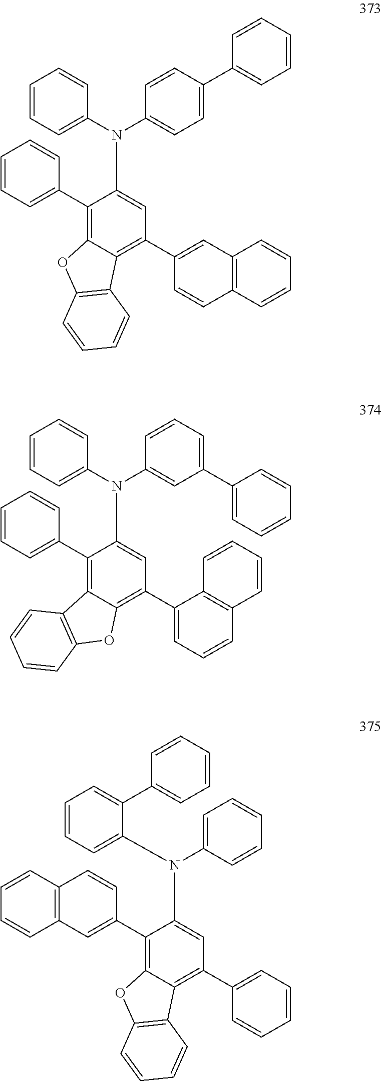 [SMILES: c1ccc(-c2ccc(N(c3ccccc3)c3cc(-c4ccc5ccccc5c4)c4c(oc5ccccc54)c3-c3ccccc3)cc2)cc1 ; c1ccc(-c2cccc(N(c3ccccc3)c3cc(-c4cccc5ccccc45)c4oc5ccccc5c4c3-c3ccccc3)c2)cc1 ; c1ccc(-c2ccccc2N(c2ccccc2)c2cc(-c3ccccc3)c3c(oc4ccccc43)c2-c2ccc3ccccc3c2)cc1]

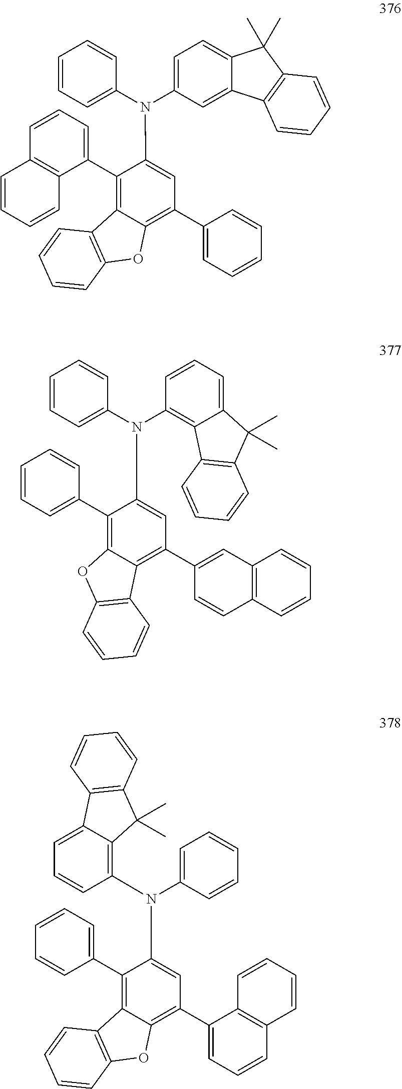 [SMILES: CC1(C)c2ccccc2-c2cc(N(c3ccccc3)c3cc(-c4ccccc4)c4oc5ccccc5c4c3-c3cccc4ccccc34)ccc21 ; CC1(C)c2ccccc2-c2c(N(c3ccccc3)c3cc(-c4ccc5ccccc5c4)c4c(oc5ccccc54)c3-c3ccccc3)cccc21 ; CC1(C)c2ccccc2-c2cccc(N(c3ccccc3)c3cc(-c4cccc5ccccc45)c4oc5ccccc5c4c3-c3ccccc3)c21]

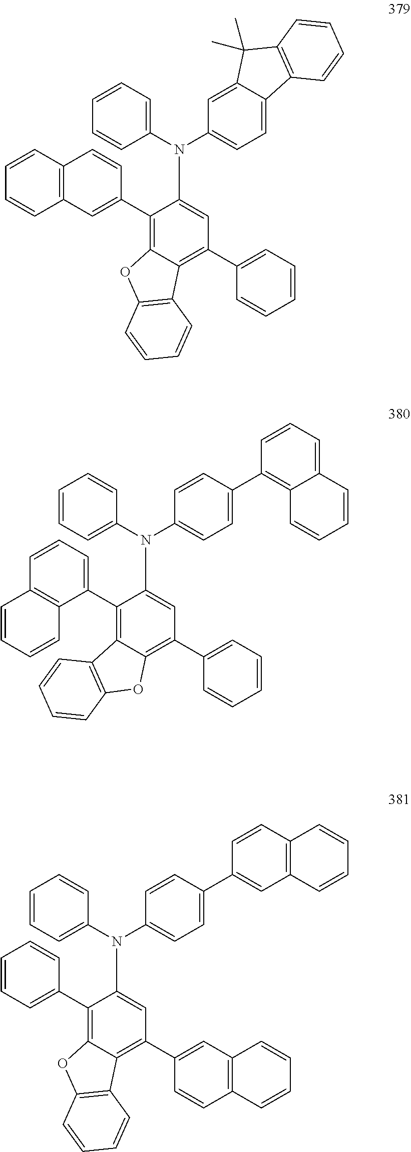 [SMILES: CC1(C)c2ccccc2-c2ccc(N(c3ccccc3)c3cc(-c4ccccc4)c4c(oc5ccccc54)c3-c3ccc4ccccc4c3)cc21 ; c1ccc(-c2cc(N(c3ccccc3)c3ccc(-c4cccc5ccccc45)cc3)c(-c3cccc4ccccc34)c3c2oc2ccccc23)cc1 ; c1ccc(-c2c(N(c3ccccc3)c3ccc(-c4ccc5ccccc5c4)cc3)cc(-c3ccc4ccccc4c3)c3c2oc2ccccc23)cc1]

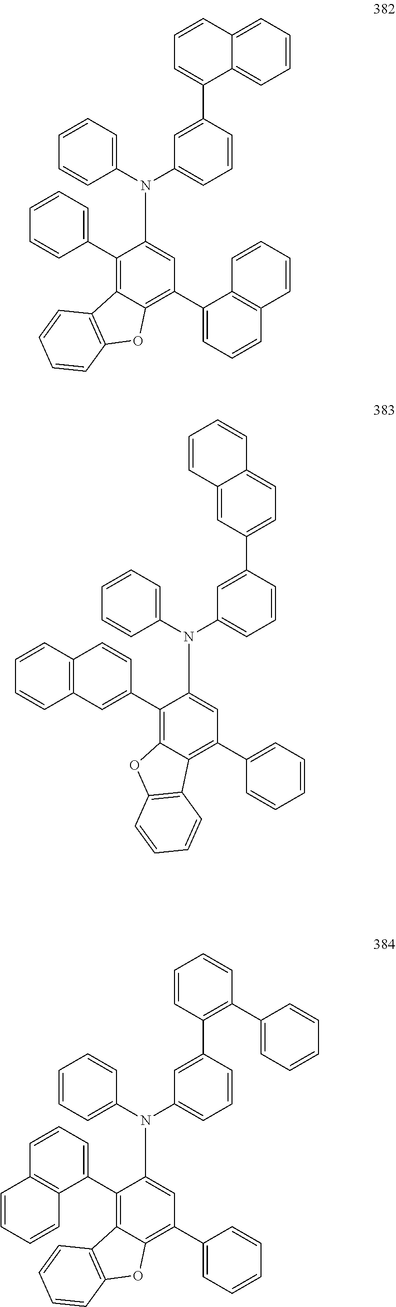 [SMILES: c1ccc(-c2c(N(c3ccccc3)c3cccc(-c4cccc5ccccc45)c3)cc(-c3cccc4ccccc34)c3oc4ccccc4c23)cc1 ; c1ccc(-c2cc(N(c3ccccc3)c3cccc(-c4ccc5ccccc5c4)c3)c(-c3ccc4ccccc4c3)c3oc4ccccc4c23)cc1 ; c1ccc(-c2ccccc2-c2cccc(N(c3ccccc3)c3cc(-c4ccccc4)c4oc5ccccc5c4c3-c3cccc4ccccc34)c2)cc1]

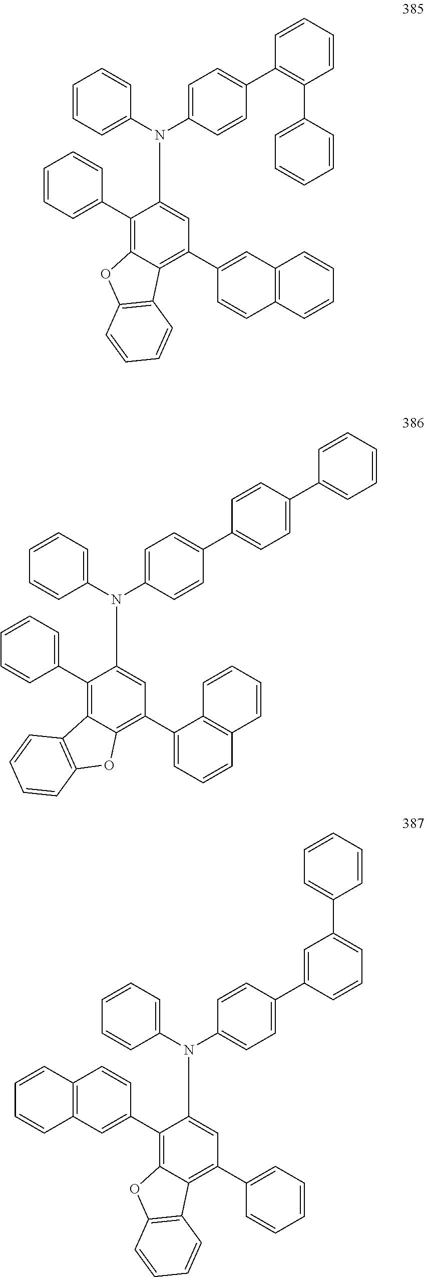 [SMILES: c1ccc(-c2ccccc2-c2ccc(N(c3ccccc3)c3cc(-c4ccc5ccccc5c4)c4c(oc5ccccc54)c3-c3ccccc3)cc2)cc1 ; c1ccc(-c2ccc(-c3ccc(N(c4ccccc4)c4cc(-c5cccc6ccccc56)c5oc6ccccc6c5c4-c4ccccc4)cc3)cc2)cc1 ; c1ccc(-c2cccc(-c3ccc(N(c4ccccc4)c4cc(-c5ccccc5)c5c(oc6ccccc65)c4-c4ccc5ccccc5c4)cc3)c2)cc1]

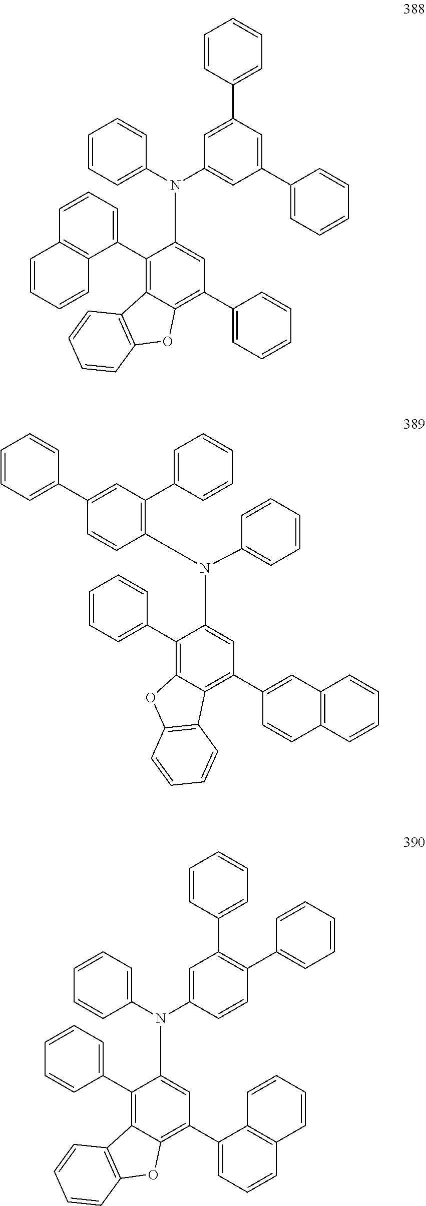 [SMILES: c1ccc(-c2cc(-c3ccccc3)cc(N(c3ccccc3)c3cc(-c4ccccc4)c4oc5ccccc5c4c3-c3cccc4ccccc34)c2)cc1 ; c1ccc(-c2ccc(N(c3ccccc3)c3cc(-c4ccc5ccccc5c4)c4c(oc5ccccc54)c3-c3ccccc3)c(-c3ccccc3)c2)cc1 ; c1ccc(-c2ccc(N(c3ccccc3)c3cc(-c4cccc5ccccc45)c4oc5ccccc5c4c3-c3ccccc3)cc2-c2ccccc2)cc1]

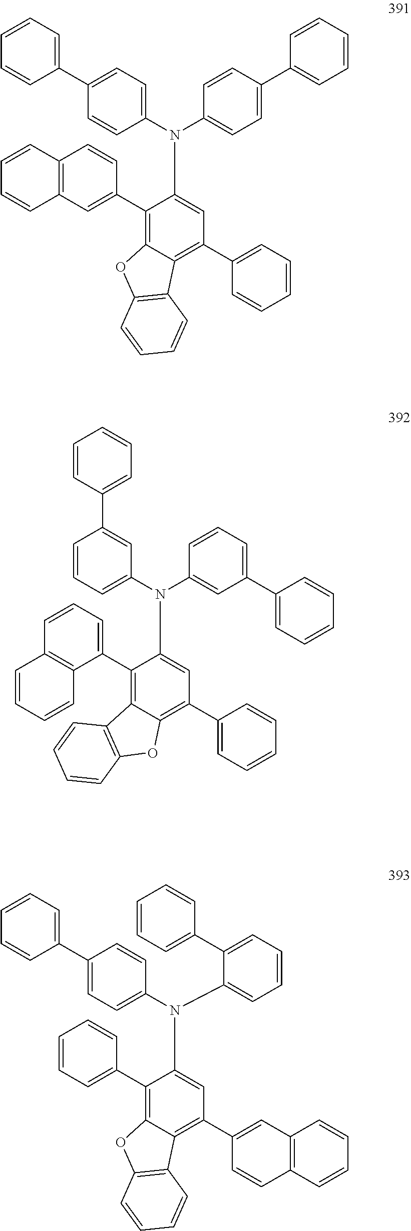 [SMILES: c1ccc(-c2ccc(N(c3ccc(-c4ccccc4)cc3)c3cc(-c4ccccc4)c4c(oc5ccccc54)c3-c3ccc4ccccc4c3)cc2)cc1 ; c1ccc(-c2cccc(N(c3cccc(-c4ccccc4)c3)c3cc(-c4ccccc4)c4oc5ccccc5c4c3-c3cccc4ccccc34)c2)cc1 ; c1ccc(-c2ccc(N(c3ccccc3-c3ccccc3)c3cc(-c4ccc5ccccc5c4)c4c(oc5ccccc54)c3-c3ccccc3)cc2)cc1]

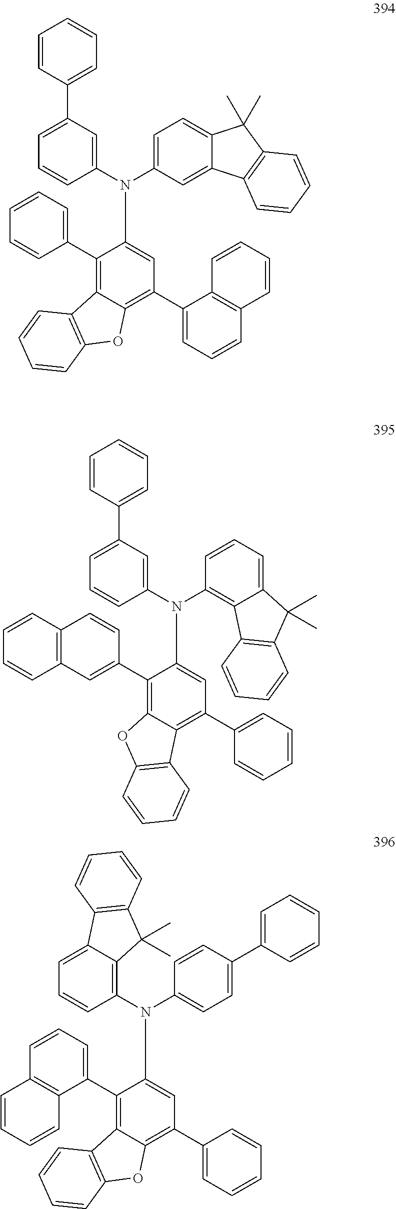

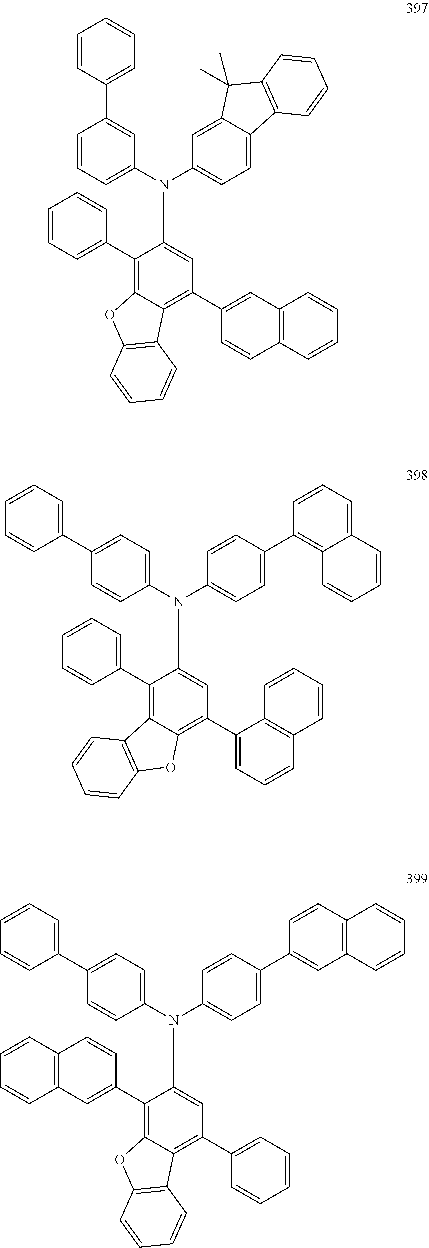 [SMILES: CC1(C)c2ccccc2-c2ccc(N(c3cccc(-c4ccccc4)c3)c3cc(-c4ccc5ccccc5c4)c4c(oc5ccccc54)c3-c3ccccc3)cc21 ; c1ccc(-c2ccc(N(c3ccc(-c4cccc5ccccc45)cc3)c3cc(-c4cccc5ccccc45)c4oc5ccccc5c4c3-c3ccccc3)cc2)cc1 ; c1ccc(-c2ccc(N(c3ccc(-c4ccc5ccccc5c4)cc3)c3cc(-c4ccccc4)c4c(oc5ccccc54)c3-c3ccc4ccccc4c3)cc2)cc1]

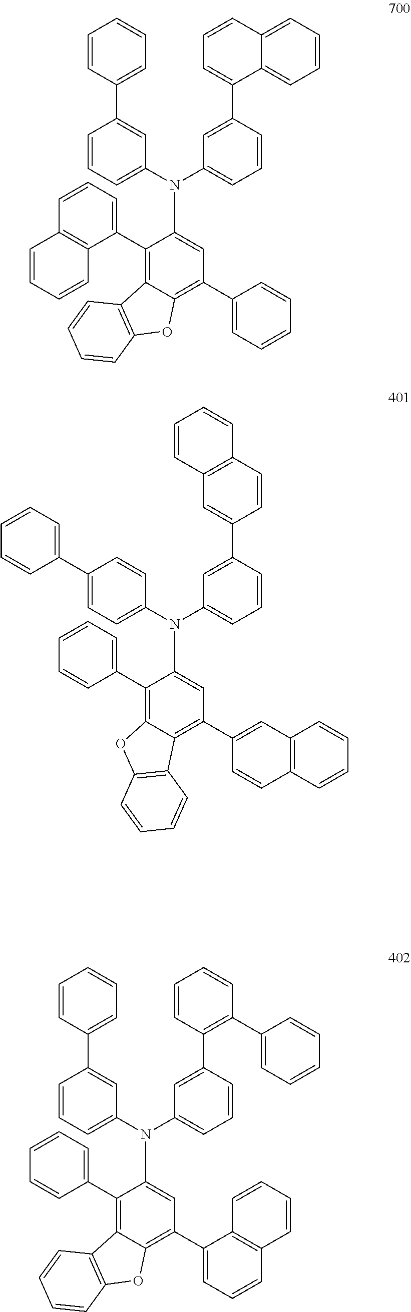 [SMILES: c1ccc(-c2cccc(N(c3cccc(-c4cccc5ccccc45)c3)c3cc(-c4ccccc4)c4oc5ccccc5c4c3-c3cccc4ccccc34)c2)cc1 ; c1ccc(-c2ccc(N(c3cccc(-c4ccc5ccccc5c4)c3)c3cc(-c4ccc5ccccc5c4)c4c(oc5ccccc54)c3-c3ccccc3)cc2)cc1 ; c1ccc(-c2cccc(N(c3cccc(-c4ccccc4-c4ccccc4)c3)c3cc(-c4cccc5ccccc45)c4oc5ccccc5c4c3-c3ccccc3)c2)cc1]

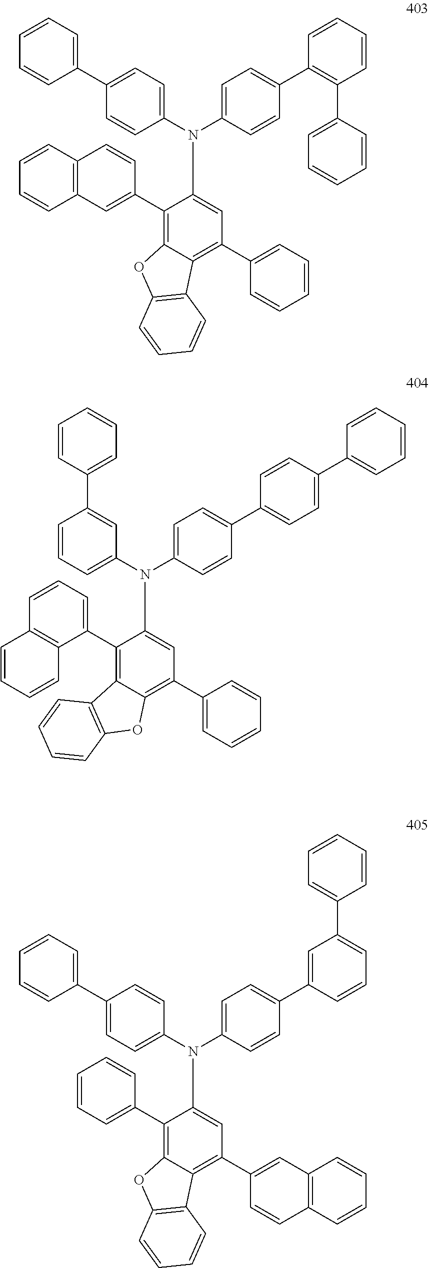 [SMILES: c1ccc(-c2ccc(N(c3ccc(-c4ccccc4-c4ccccc4)cc3)c3cc(-c4ccccc4)c4c(oc5ccccc54)c3-c3ccc4ccccc4c3)cc2)cc1 ; c1ccc(-c2ccc(-c3ccc(N(c4cccc(-c5ccccc5)c4)c4cc(-c5ccccc5)c5oc6ccccc6c5c4-c4cccc5ccccc45)cc3)cc2)cc1 ; c1ccc(-c2ccc(N(c3ccc(-c4cccc(-c5ccccc5)c4)cc3)c3cc(-c4ccc5ccccc5c4)c4c(oc5ccccc54)c3-c3ccccc3)cc2)cc1]

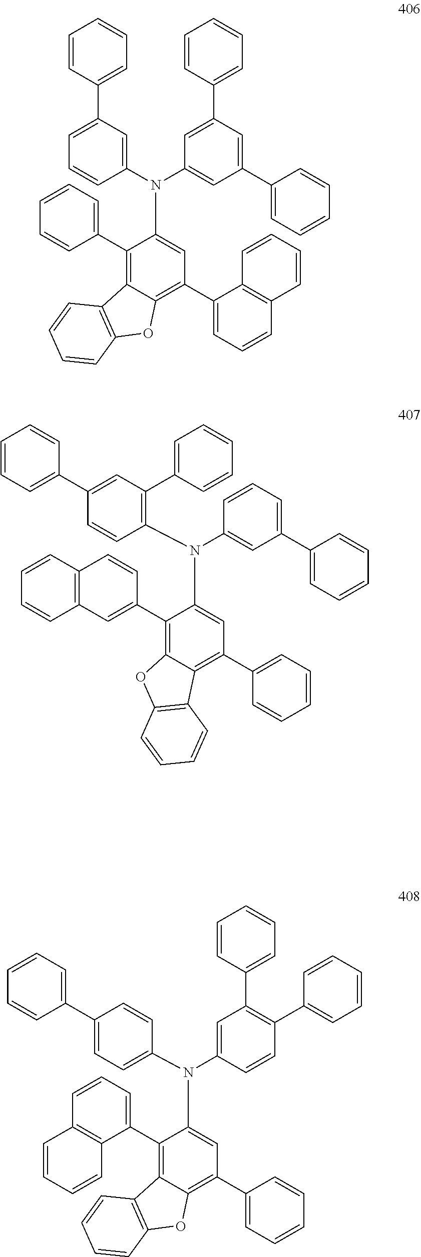 [SMILES: c1ccc(-c2cccc(N(c3cc(-c4ccccc4)cc(-c4ccccc4)c3)c3cc(-c4cccc5ccccc45)c4oc5ccccc5c4c3-c3ccccc3)c2)cc1 ; c1ccc(-c2cccc(N(c3ccc(-c4ccccc4)cc3-c3ccccc3)c3cc(-c4ccccc4)c4c(oc5ccccc54)c3-c3ccc4ccccc4c3)c2)cc1 ; c1ccc(-c2ccc(N(c3ccc(-c4ccccc4)c(-c4ccccc4)c3)c3cc(-c4ccccc4)c4oc5ccccc5c4c3-c3cccc4ccccc34)cc2)cc1]

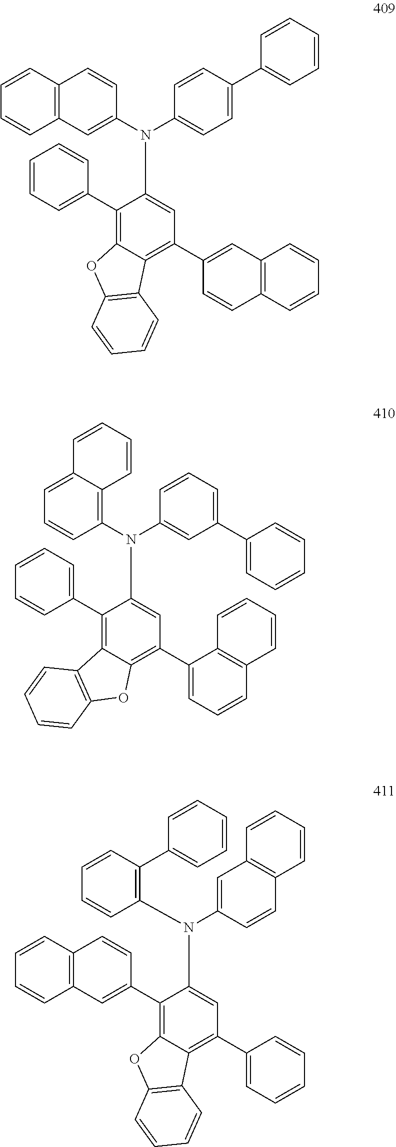 [SMILES: c1ccc(-c2ccc(N(c3ccc4ccccc4c3)c3cc(-c4ccc5ccccc5c4)c4c(oc5ccccc54)c3-c3ccccc3)cc2)cc1 ; c1ccc(-c2cccc(N(c3cc(-c4cccc5ccccc45)c4oc5ccccc5c4c3-c3ccccc3)c3cccc4ccccc34)c2)cc1 ; c1ccc(-c2ccccc2N(c2ccc3ccccc3c2)c2cc(-c3ccccc3)c3c(oc4ccccc43)c2-c2ccc3ccccc3c2)cc1]

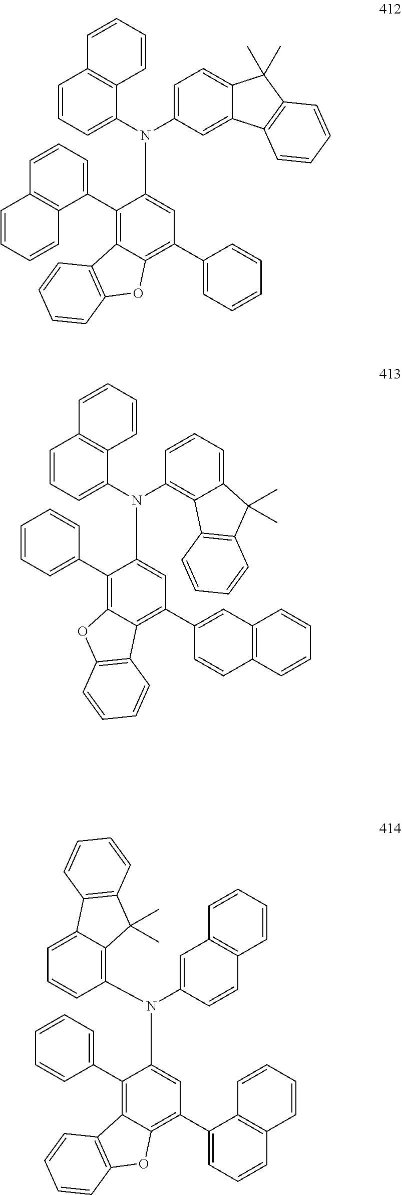 [SMILES: CC1(C)c2ccccc2-c2cc(N(c3cc(-c4ccccc4)c4oc5ccccc5c4c3-c3cccc4ccccc34)c3cccc4ccccc34)ccc21 ; CC1(C)c2ccccc2-c2c(N(c3cc(-c4ccc5ccccc5c4)c4c(oc5ccccc54)c3-c3ccccc3)c3cccc4ccccc34)cccc21 ; CC1(C)c2ccccc2-c2cccc(N(c3ccc4ccccc4c3)c3cc(-c4cccc5ccccc45)c4oc5ccccc5c4c3-c3ccccc3)c21]

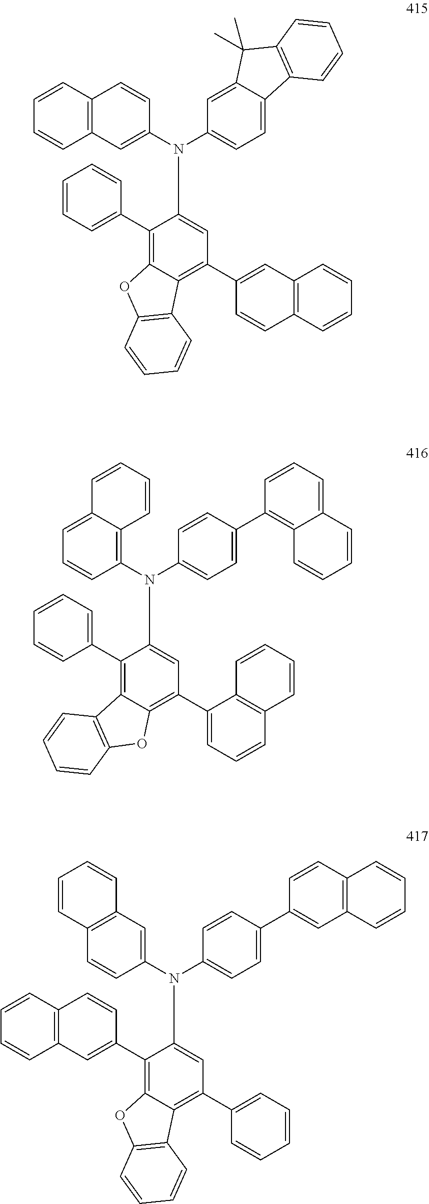 [SMILES: CC1(C)c2ccccc2-c2ccc(N(c3ccc4ccccc4c3)c3cc(-c4ccc5ccccc5c4)c4c(oc5ccccc54)c3-c3ccccc3)cc21 ; c1ccc(-c2c(N(c3ccc(-c4cccc5ccccc45)cc3)c3cccc4ccccc34)cc(-c3cccc4ccccc34)c3oc4ccccc4c23)cc1 ; c1ccc(-c2cc(N(c3ccc(-c4ccc5ccccc5c4)cc3)c3ccc4ccccc4c3)c(-c3ccc4ccccc4c3)c3oc4ccccc4c23)cc1]

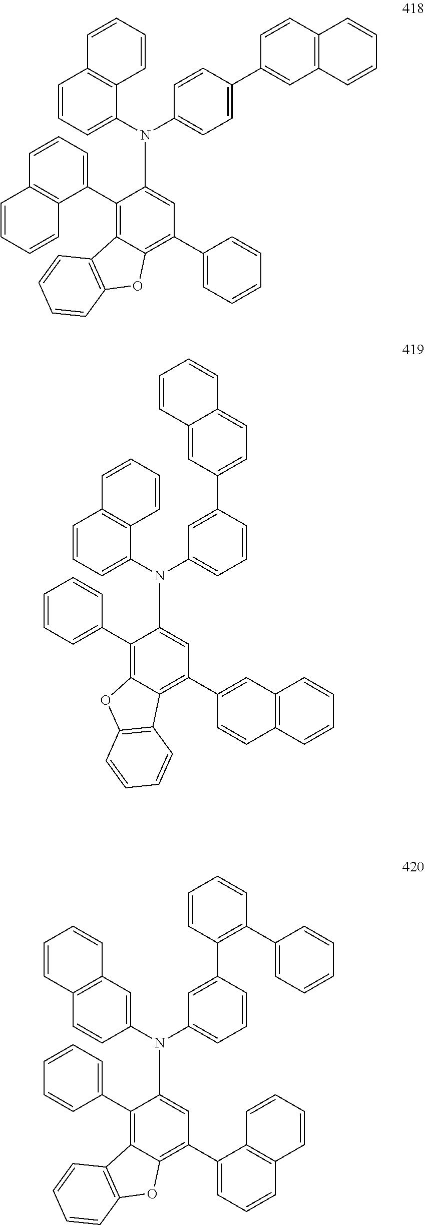 [SMILES: c1ccc(-c2cc(N(c3ccc(-c4ccc5ccccc5c4)cc3)c3cccc4ccccc34)c(-c3cccc4ccccc34)c3c2oc2ccccc23)cc1 ; c1ccc(-c2c(N(c3cccc(-c4ccc5ccccc5c4)c3)c3cccc4ccccc34)cc(-c3ccc4ccccc4c3)c3c2oc2ccccc23)cc1 ; c1ccc(-c2ccccc2-c2cccc(N(c3ccc4ccccc4c3)c3cc(-c4cccc5ccccc45)c4oc5ccccc5c4c3-c3ccccc3)c2)cc1]

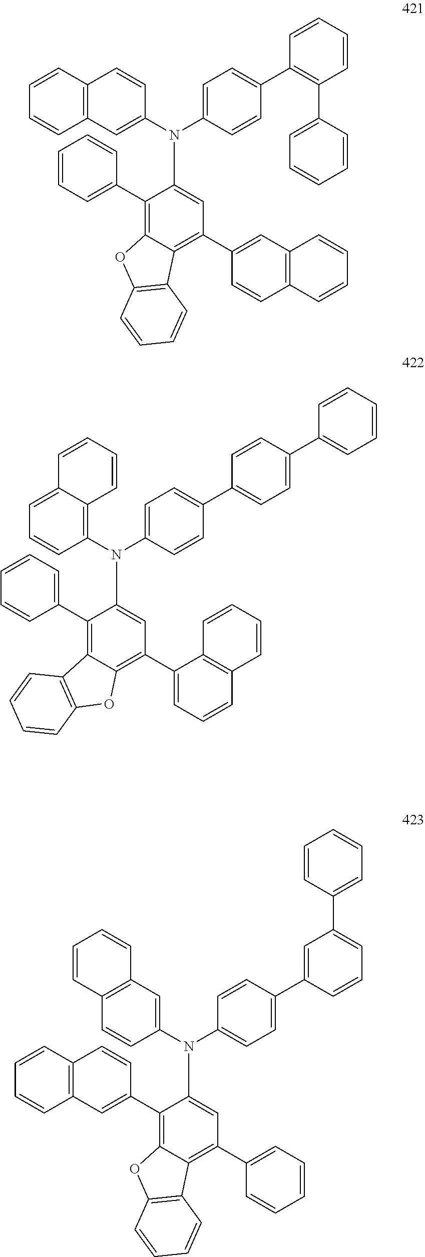 [SMILES: c1ccc(-c2ccccc2-c2ccc(N(c3ccc4ccccc4c3)c3cc(-c4ccc5ccccc5c4)c4c(oc5ccccc54)c3-c3ccccc3)cc2)cc1 ; c1ccc(-c2ccc(-c3ccc(N(c4cc(-c5cccc6ccccc56)c5oc6ccccc6c5c4-c4ccccc4)c4cccc5ccccc45)cc3)cc2)cc1 ; c1ccc(-c2cccc(-c3ccc(N(c4ccc5ccccc5c4)c4cc(-c5ccccc5)c5c(oc6ccccc65)c4-c4ccc5ccccc5c4)cc3)c2)cc1]

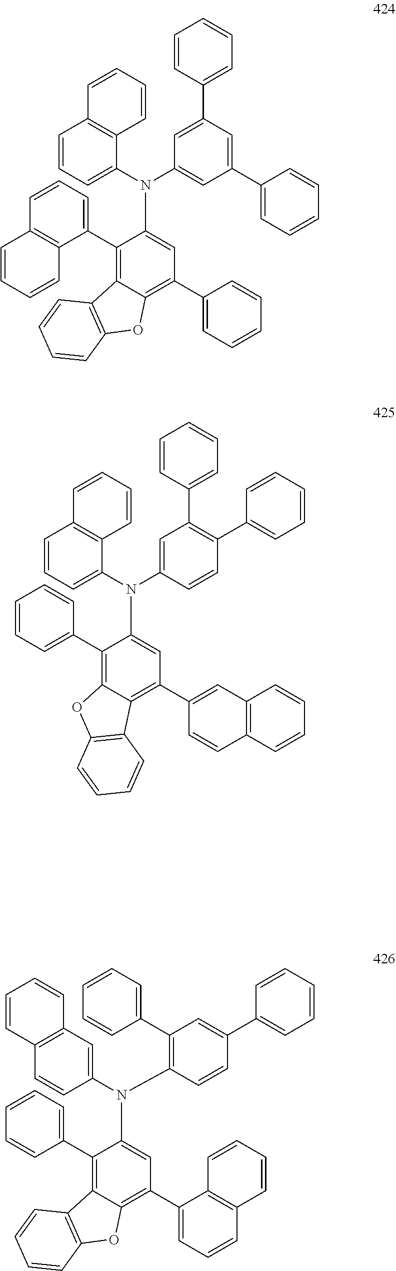 [SMILES: c1ccc(-c2cc(-c3ccccc3)cc(N(c3cc(-c4ccccc4)c4oc5ccccc5c4c3-c3cccc4ccccc34)c3cccc4ccccc34)c2)cc1 ; c1ccc(-c2ccc(N(c3cc(-c4ccc5ccccc5c4)c4c(oc5ccccc54)c3-c3ccccc3)c3cccc4ccccc34)cc2-c2ccccc2)cc1 ; c1ccc(-c2ccc(N(c3ccc4ccccc4c3)c3cc(-c4cccc5ccccc45)c4oc5ccccc5c4c3-c3ccccc3)c(-c3ccccc3)c2)cc1]

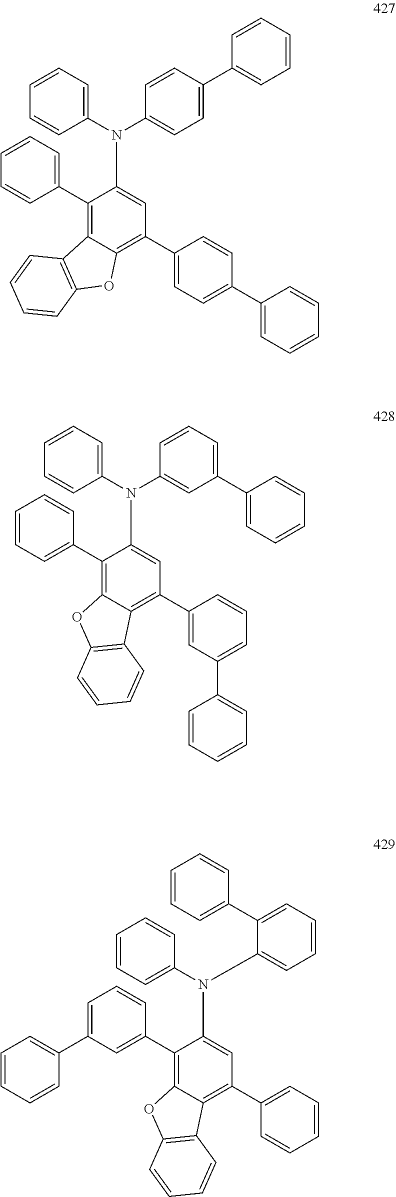 [SMILES: c1ccc(-c2ccc(-c3cc(N(c4ccccc4)c4ccc(-c5ccccc5)cc4)c(-c4ccccc4)c4c3oc3ccccc34)cc2)cc1 ; c1ccc(-c2cccc(-c3cc(N(c4ccccc4)c4cccc(-c5ccccc5)c4)c(-c4ccccc4)c4oc5ccccc5c34)c2)cc1 ; c1ccc(-c2cccc(-c3c(N(c4ccccc4)c4ccccc4-c4ccccc4)cc(-c4ccccc4)c4c3oc3ccccc34)c2)cc1]

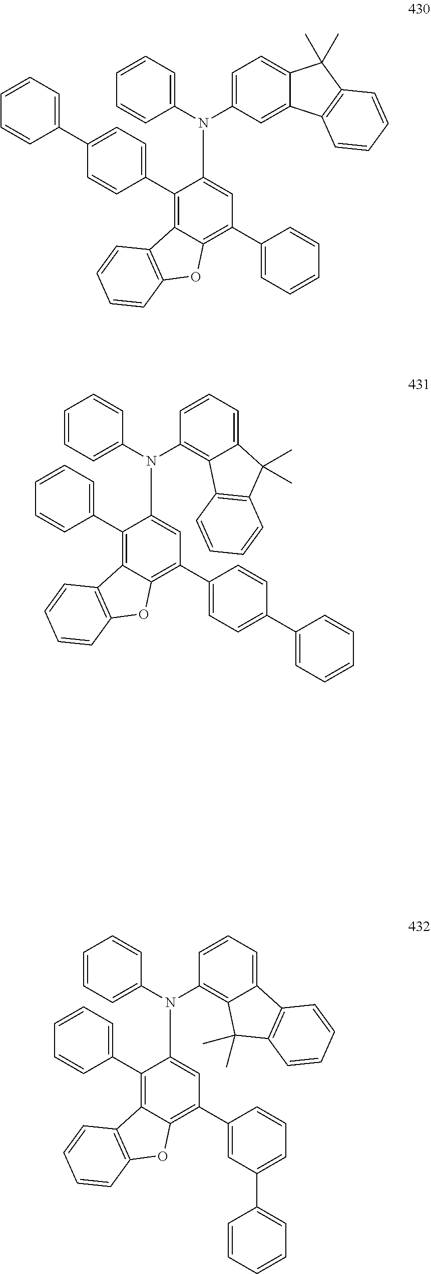 [SMILES: CC1(C)c2ccccc2-c2cc(N(c3ccccc3)c3cc(-c4ccccc4)c4oc5ccccc5c4c3-c3ccc(-c4ccccc4)cc3)ccc21 ; CC1(C)c2ccccc2-c2c(N(c3ccccc3)c3cc(-c4ccc(-c5ccccc5)cc4)c4oc5ccccc5c4c3-c3ccccc3)cccc21 ; CC1(C)c2ccccc2-c2cccc(N(c3ccccc3)c3cc(-c4cccc(-c5ccccc5)c4)c4oc5ccccc5c4c3-c3ccccc3)c21]

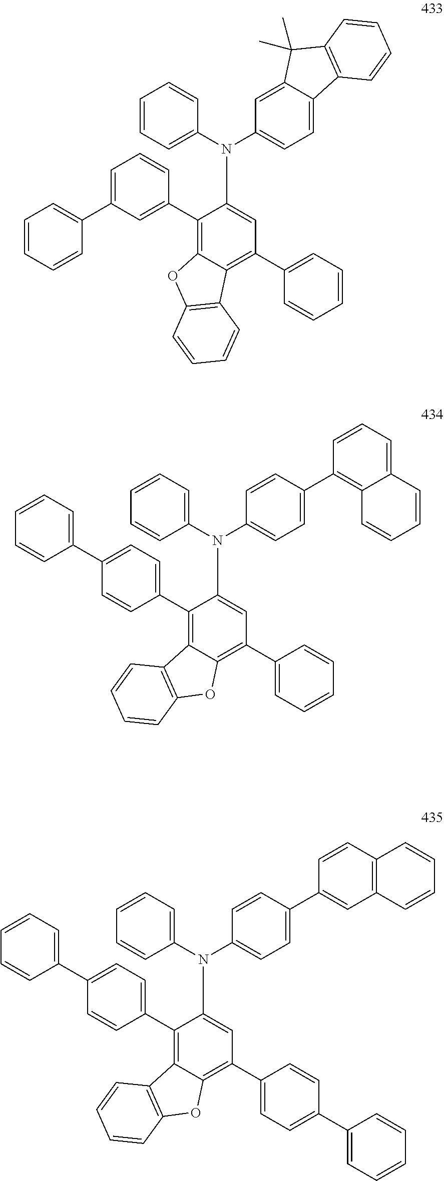 [SMILES: CC1(C)c2ccccc2-c2ccc(N(c3ccccc3)c3cc(-c4ccccc4)c4c(oc5ccccc54)c3-c3cccc(-c4ccccc4)c3)cc21 ; c1ccc(-c2ccc(-c3c(N(c4ccccc4)c4ccc(-c5cccc6ccccc56)cc4)cc(-c4ccccc4)c4oc5ccccc5c34)cc2)cc1 ; c1ccc(-c2ccc(-c3cc(N(c4ccccc4)c4ccc(-c5ccc6ccccc6c5)cc4)c(-c4ccc(-c5ccccc5)cc4)c4c3oc3ccccc34)cc2)cc1]

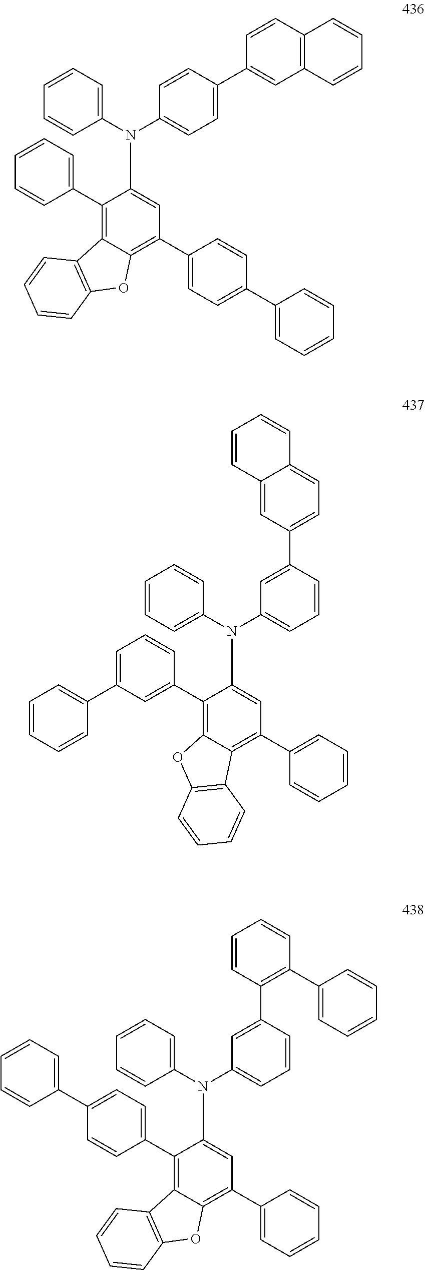 [SMILES: c1ccc(-c2ccc(-c3cc(N(c4ccccc4)c4ccc(-c5ccc6ccccc6c5)cc4)c(-c4ccccc4)c4c3oc3ccccc34)cc2)cc1 ; c1ccc(-c2cccc(-c3c(N(c4ccccc4)c4cccc(-c5ccc6ccccc6c5)c4)cc(-c4ccccc4)c4c3oc3ccccc34)c2)cc1 ; c1ccc(-c2ccc(-c3c(N(c4ccccc4)c4cccc(-c5ccccc5-c5ccccc5)c4)cc(-c4ccccc4)c4oc5ccccc5c34)cc2)cc1]

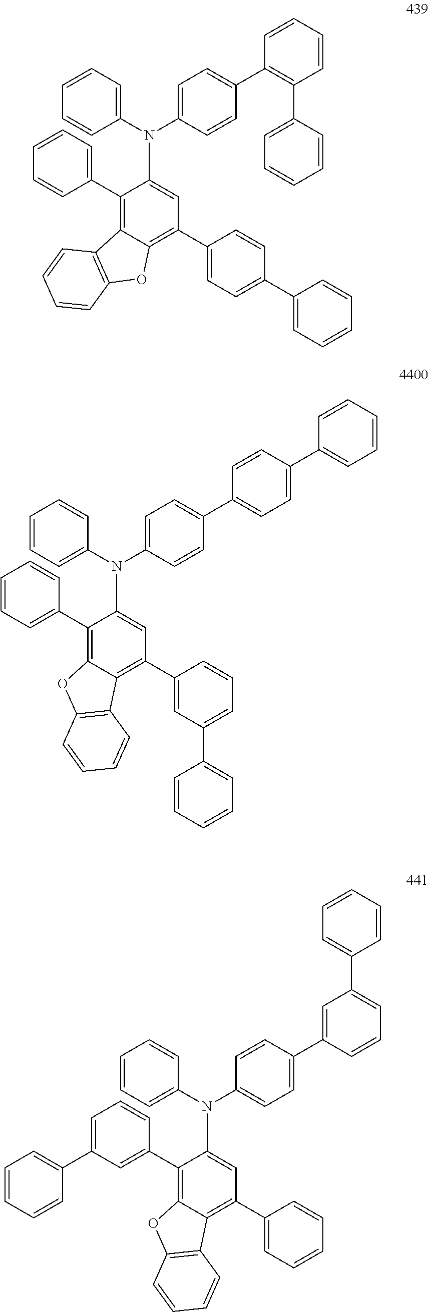 [SMILES: c1ccc(-c2ccc(-c3cc(N(c4ccccc4)c4ccc(-c5ccccc5-c5ccccc5)cc4)c(-c4ccccc4)c4c3oc3ccccc34)cc2)cc1 ; c1ccc(-c2ccc(-c3ccc(N(c4ccccc4)c4cc(-c5cccc(-c6ccccc6)c5)c5c(oc6ccccc65)c4-c4ccccc4)cc3)cc2)cc1 ; c1ccc(-c2cccc(-c3ccc(N(c4ccccc4)c4cc(-c5ccccc5)c5c(oc6ccccc65)c4-c4cccc(-c5ccccc5)c4)cc3)c2)cc1]

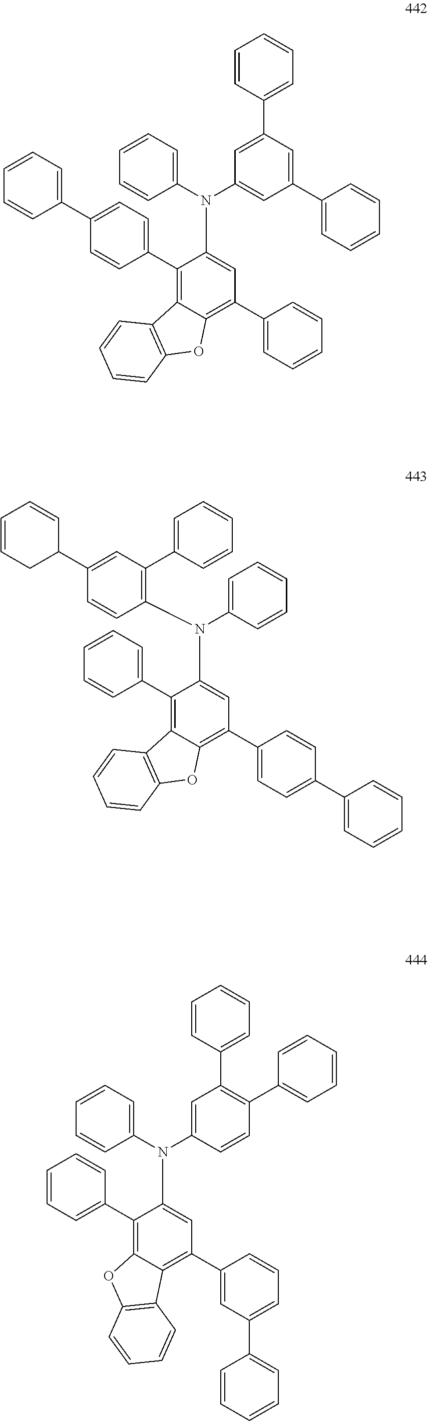 [SMILES: c1ccc(-c2ccc(-c3c(N(c4ccccc4)c4cc(-c5ccccc5)cc(-c5ccccc5)c4)cc(-c4ccccc4)c4oc5ccccc5c34)cc2)cc1 ; C1=CCC(c2ccc(N(c3ccccc3)c3cc(-c4ccc(-c5ccccc5)cc4)c4oc5ccccc5c4c3-c3ccccc3)c(-c3ccccc3)c2)C=C1 ; c1ccc(-c2cccc(-c3cc(N(c4ccccc4)c4ccc(-c5ccccc5)c(-c5ccccc5)c4)c(-c4ccccc4)c4oc5ccccc5c34)c2)cc1]

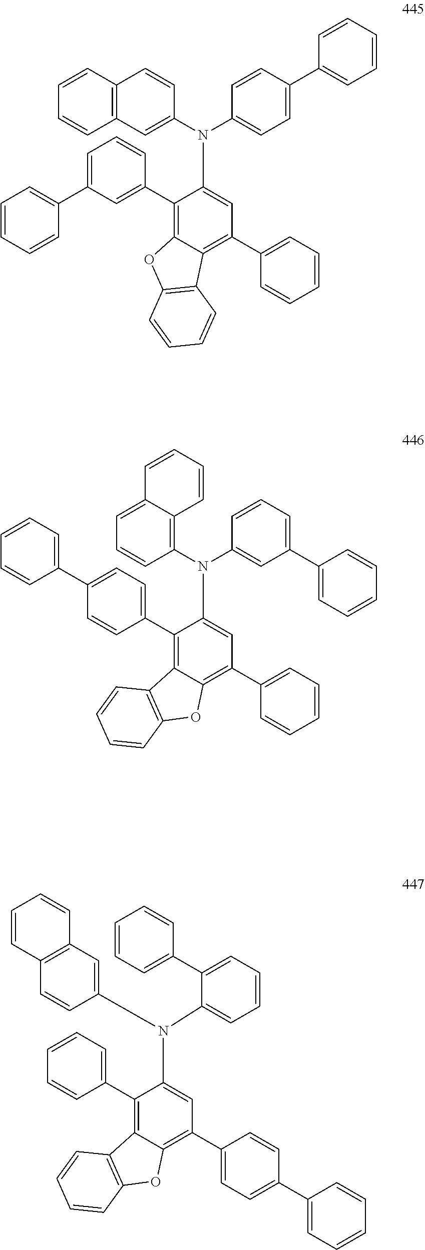 [SMILES: c1ccc(-c2ccc(N(c3ccc4ccccc4c3)c3cc(-c4ccccc4)c4c(oc5ccccc54)c3-c3cccc(-c4ccccc4)c3)cc2)cc1 ; c1ccc(-c2ccc(-c3c(N(c4cccc(-c5ccccc5)c4)c4cccc5ccccc45)cc(-c4ccccc4)c4oc5ccccc5c34)cc2)cc1 ; c1ccc(-c2ccc(-c3cc(N(c4ccc5ccccc5c4)c4ccccc4-c4ccccc4)c(-c4ccccc4)c4c3oc3ccccc34)cc2)cc1]

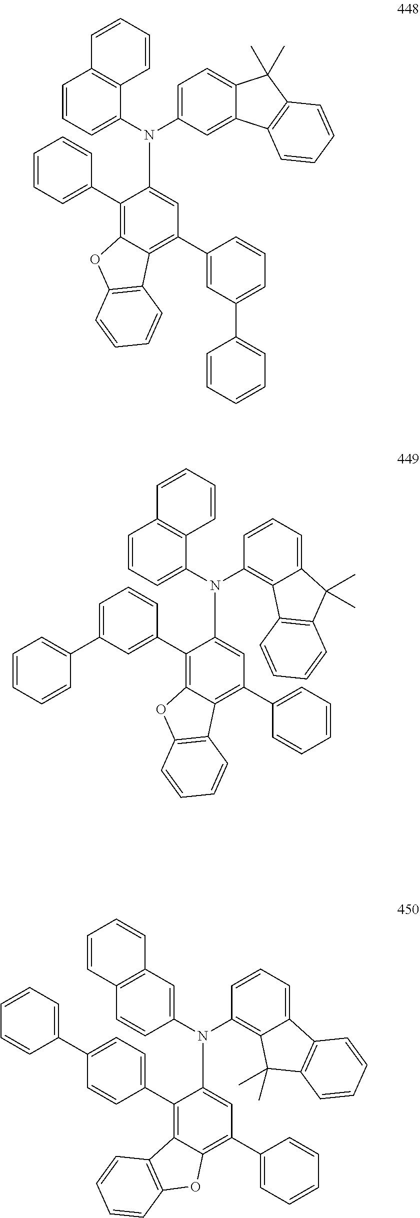 [SMILES: CC1(C)c2ccccc2-c2cc(N(c3cc(-c4cccc(-c5ccccc5)c4)c4c(oc5ccccc54)c3-c3ccccc3)c3cccc4ccccc34)ccc21 ; CC1(C)c2ccccc2-c2c(N(c3cc(-c4ccccc4)c4c(oc5ccccc54)c3-c3cccc(-c4ccccc4)c3)c3cccc4ccccc34)cccc21 ; CC1(C)c2ccccc2-c2cccc(N(c3ccc4ccccc4c3)c3cc(-c4ccccc4)c4oc5ccccc5c4c3-c3ccc(-c4ccccc4)cc3)c21]

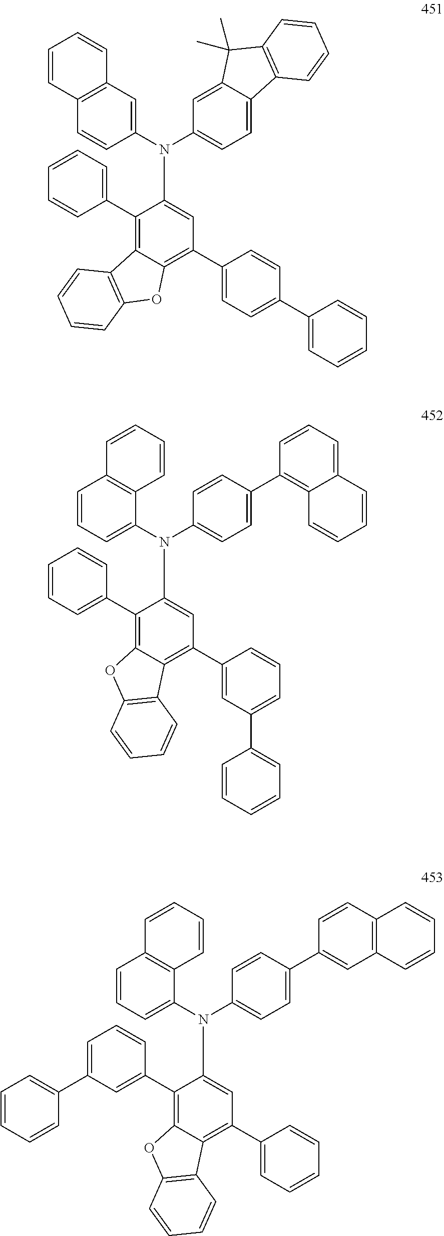 [SMILES: CC1(C)c2ccccc2-c2ccc(N(c3ccc4ccccc4c3)c3cc(-c4ccc(-c5ccccc5)cc4)c4oc5ccccc5c4c3-c3ccccc3)cc21 ; c1ccc(-c2cccc(-c3cc(N(c4ccc(-c5cccc6ccccc56)cc4)c4cccc5ccccc45)c(-c4ccccc4)c4oc5ccccc5c34)c2)cc1 ; c1ccc(-c2cccc(-c3c(N(c4ccc(-c5ccc6ccccc6c5)cc4)c4cccc5ccccc45)cc(-c4ccccc4)c4c3oc3ccccc34)c2)cc1]

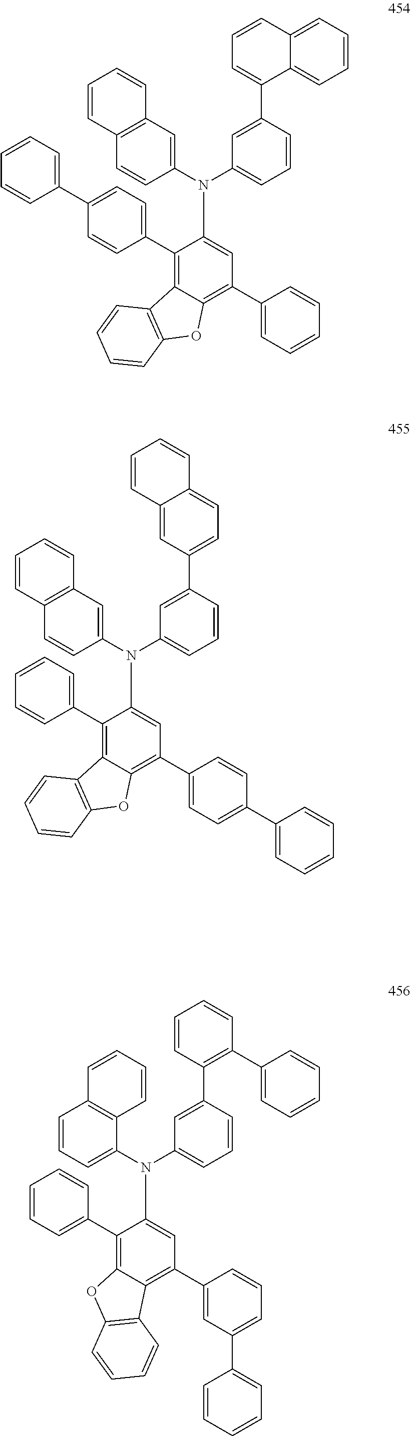 [SMILES: c1ccc(-c2ccc(-c3c(N(c4cccc(-c5cccc6ccccc56)c4)c4ccc5ccccc5c4)cc(-c4ccccc4)c4oc5ccccc5c34)cc2)cc1 ; c1ccc(-c2ccc(-c3cc(N(c4cccc(-c5ccc6ccccc6c5)c4)c4ccc5ccccc5c4)c(-c4ccccc4)c4c3oc3ccccc34)cc2)cc1 ; c1ccc(-c2cccc(-c3cc(N(c4cccc(-c5ccccc5-c5ccccc5)c4)c4cccc5ccccc45)c(-c4ccccc4)c4oc5ccccc5c34)c2)cc1]

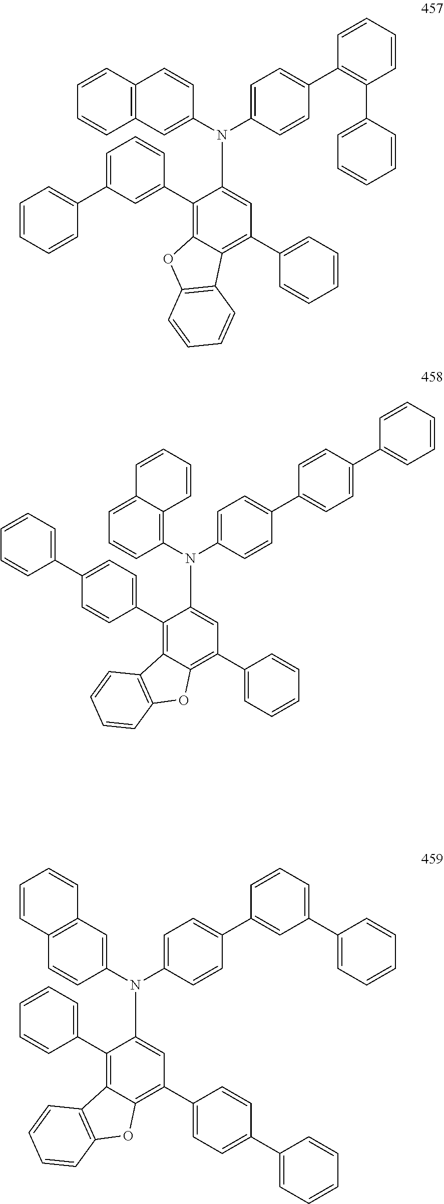 [SMILES: c1ccc(-c2cccc(-c3c(N(c4ccc(-c5ccccc5-c5ccccc5)cc4)c4ccc5ccccc5c4)cc(-c4ccccc4)c4c3oc3ccccc34)c2)cc1 ; c1ccc(-c2ccc(-c3ccc(N(c4cc(-c5ccccc5)c5oc6ccccc6c5c4-c4ccc(-c5ccccc5)cc4)c4cccc5ccccc45)cc3)cc2)cc1 ; c1ccc(-c2ccc(-c3cc(N(c4ccc(-c5cccc(-c6ccccc6)c5)cc4)c4ccc5ccccc5c4)c(-c4ccccc4)c4c3oc3ccccc34)cc2)cc1]

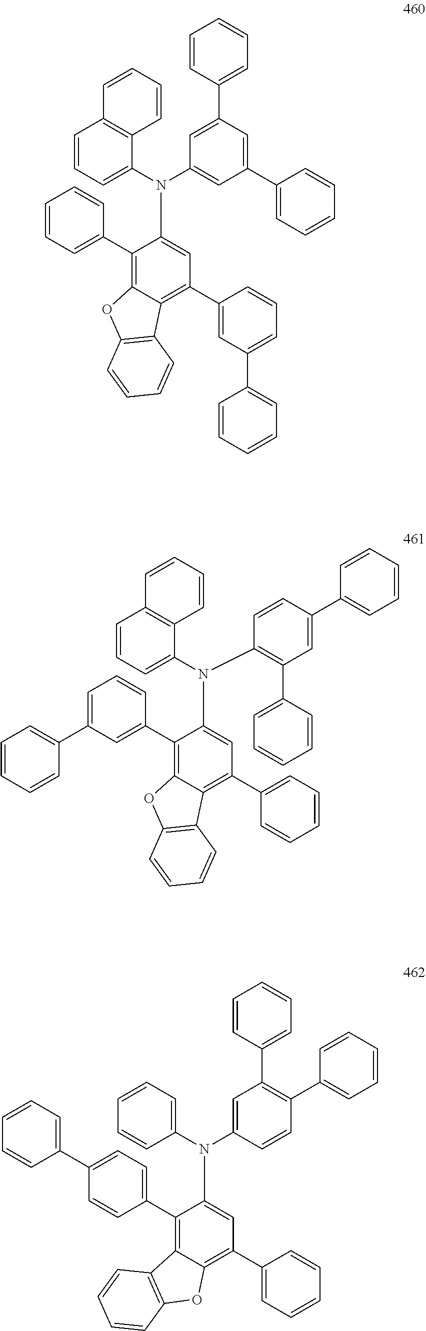 [SMILES: c1ccc(-c2cccc(-c3cc(N(c4cc(-c5ccccc5)cc(-c5ccccc5)c4)c4cccc5ccccc45)c(-c4ccccc4)c4oc5ccccc5c34)c2)cc1 ; c1ccc(-c2cccc(-c3c(N(c4ccc(-c5ccccc5)cc4-c4ccccc4)c4cccc5ccccc45)cc(-c4ccccc4)c4c3oc3ccccc34)c2)cc1 ; c1ccc(-c2ccc(-c3c(N(c4ccccc4)c4ccc(-c5ccccc5)c(-c5ccccc5)c4)cc(-c4ccccc4)c4oc5ccccc5c34)cc2)cc1]

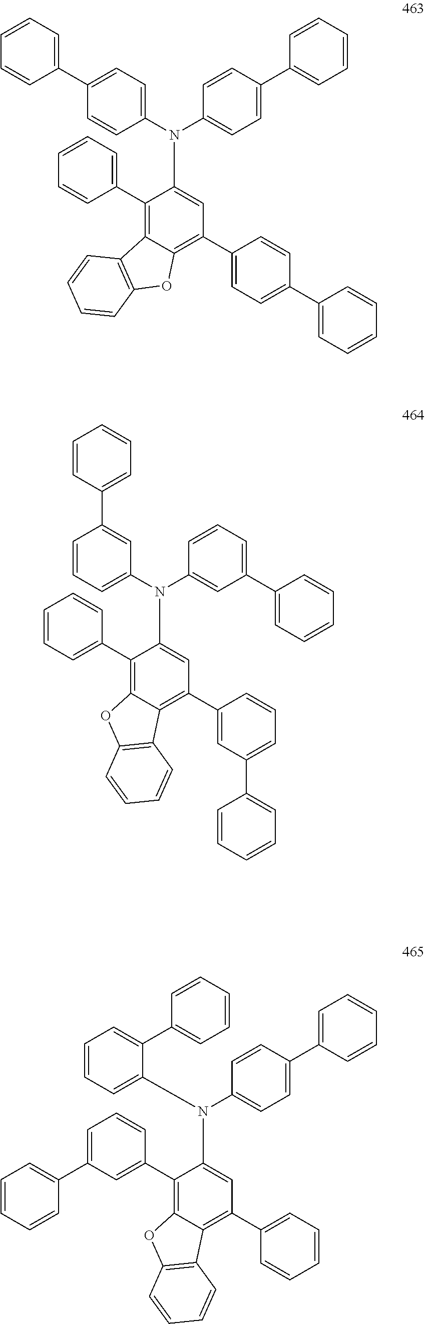 [SMILES: c1ccc(-c2ccc(-c3cc(N(c4ccc(-c5ccccc5)cc4)c4ccc(-c5ccccc5)cc4)c(-c4ccccc4)c4c3oc3ccccc34)cc2)cc1 ; c1ccc(-c2cccc(-c3cc(N(c4cccc(-c5ccccc5)c4)c4cccc(-c5ccccc5)c4)c(-c4ccccc4)c4oc5ccccc5c34)c2)cc1 ; c1ccc(-c2ccc(N(c3ccccc3-c3ccccc3)c3cc(-c4ccccc4)c4c(oc5ccccc54)c3-c3cccc(-c4ccccc4)c3)cc2)cc1]

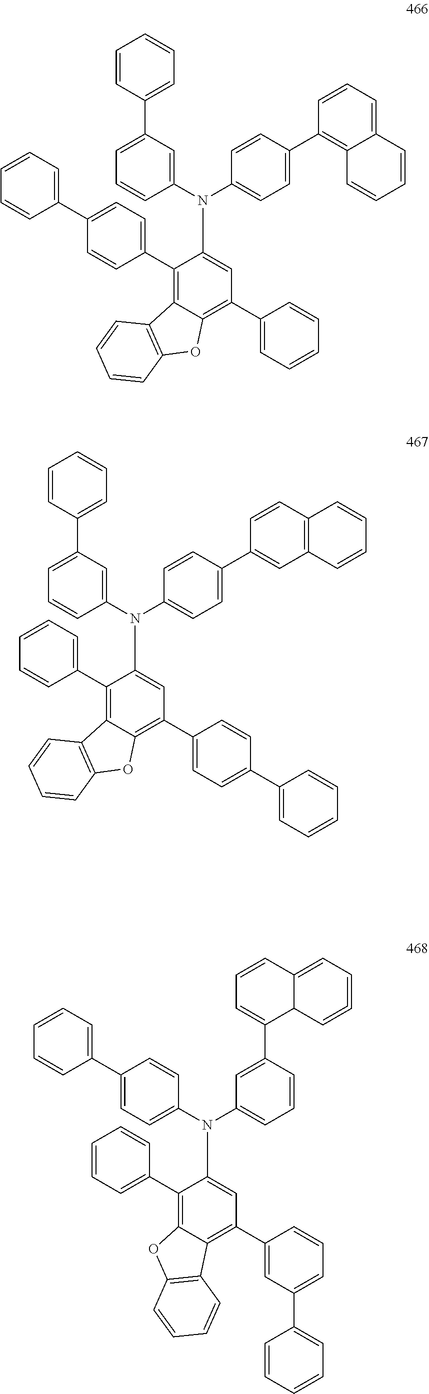 [SMILES: c1ccc(-c2ccc(-c3c(N(c4ccc(-c5cccc6ccccc56)cc4)c4cccc(-c5ccccc5)c4)cc(-c4ccccc4)c4oc5ccccc5c34)cc2)cc1 ; c1ccc(-c2ccc(-c3cc(N(c4ccc(-c5ccc6ccccc6c5)cc4)c4cccc(-c5ccccc5)c4)c(-c4ccccc4)c4c3oc3ccccc34)cc2)cc1 ; C1=CC=C2C=CC=C(c3cccc(N(c4ccc(-c5ccccc5)cc4)c4cc(-c5cccc(-c6ccccc6)c5)c5c(oc6ccccc65)c4-c4ccccc4)c3)C2C=1]

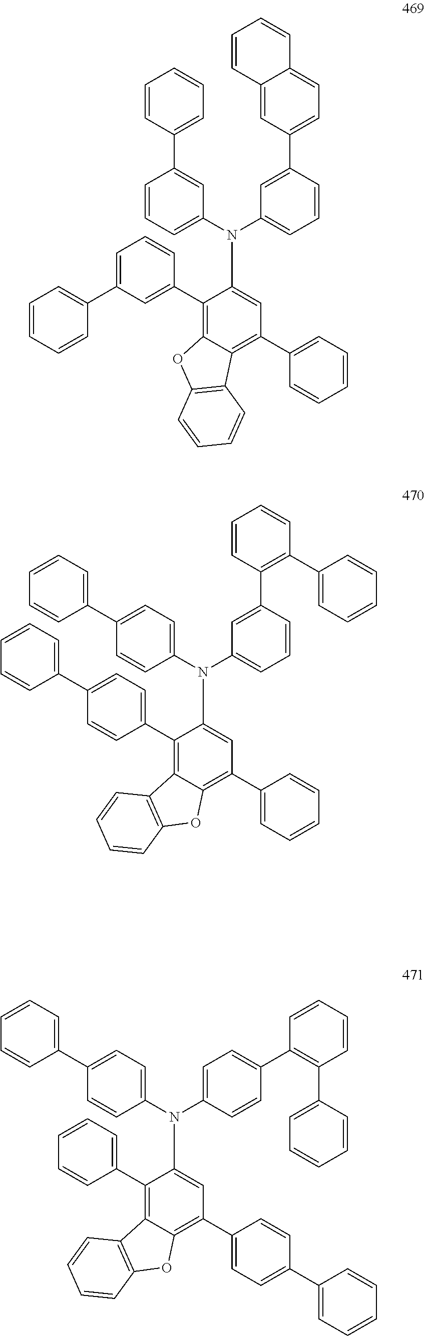 [SMILES: c1ccc(-c2cccc(-c3c(N(c4cccc(-c5ccccc5)c4)c4cccc(-c5ccc6ccccc6c5)c4)cc(-c4ccccc4)c4c3oc3ccccc34)c2)cc1 ; c1ccc(-c2ccc(-c3c(N(c4ccc(-c5ccccc5)cc4)c4cccc(-c5ccccc5-c5ccccc5)c4)cc(-c4ccccc4)c4oc5ccccc5c34)cc2)cc1 ; c1ccc(-c2ccc(-c3cc(N(c4ccc(-c5ccccc5)cc4)c4ccc(-c5ccccc5-c5ccccc5)cc4)c(-c4ccccc4)c4c3oc3ccccc34)cc2)cc1]

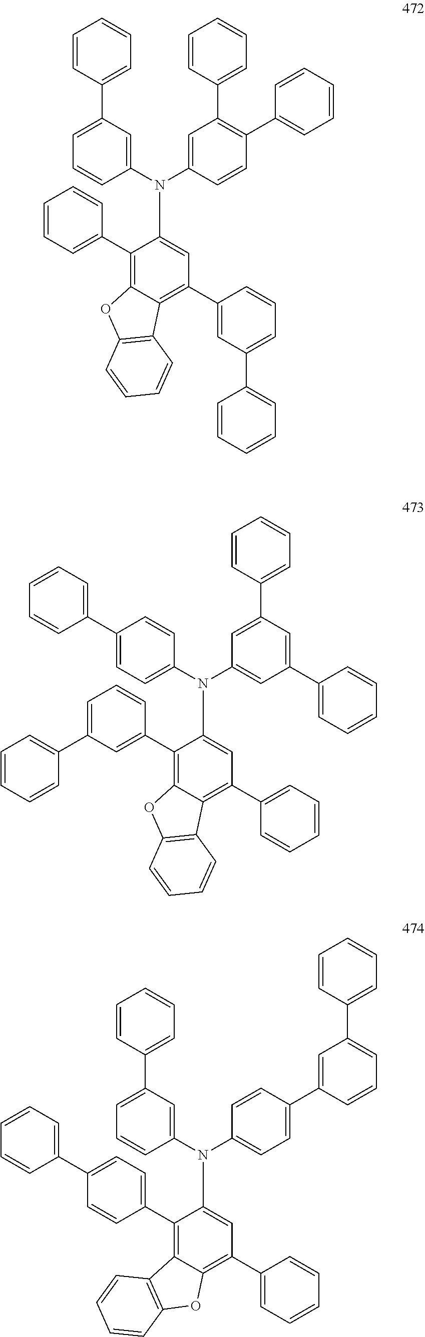 [SMILES: c1ccc(-c2cccc(-c3cc(N(c4cccc(-c5ccccc5)c4)c4ccc(-c5ccccc5)c(-c5ccccc5)c4)c(-c4ccccc4)c4oc5ccccc5c34)c2)cc1 ; c1ccc(-c2ccc(N(c3cc(-c4ccccc4)cc(-c4ccccc4)c3)c3cc(-c4ccccc4)c4c(oc5ccccc54)c3-c3cccc(-c4ccccc4)c3)cc2)cc1 ; c1ccc(-c2ccc(-c3c(N(c4ccc(-c5cccc(-c6ccccc6)c5)cc4)c4cccc(-c5ccccc5)c4)cc(-c4ccccc4)c4oc5ccccc5c34)cc2)cc1]

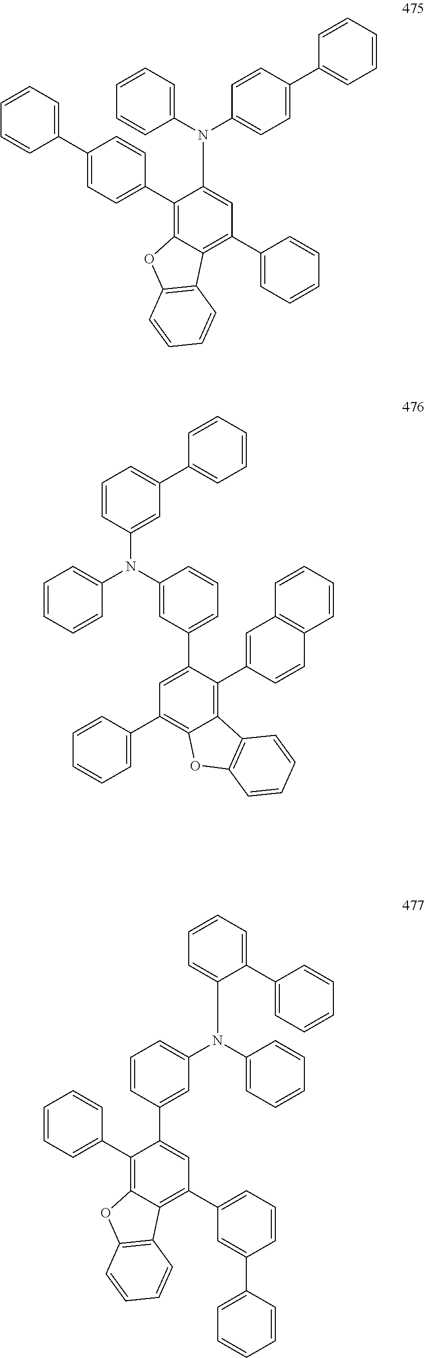 [SMILES: c1ccc(-c2ccc(-c3c(N(c4ccccc4)c4ccc(-c5ccccc5)cc4)cc(-c4ccccc4)c4c3oc3ccccc34)cc2)cc1 ; c1ccc(-c2cccc(N(c3ccccc3)c3cccc(-c4cc(-c5ccccc5)c5oc6ccccc6c5c4-c4ccc5ccccc5c4)c3)c2)cc1 ; c1ccc(-c2cccc(-c3cc(-c4cccc(N(c5ccccc5)c5ccccc5-c5ccccc5)c4)c(-c4ccccc4)c4oc5ccccc5c34)c2)cc1]

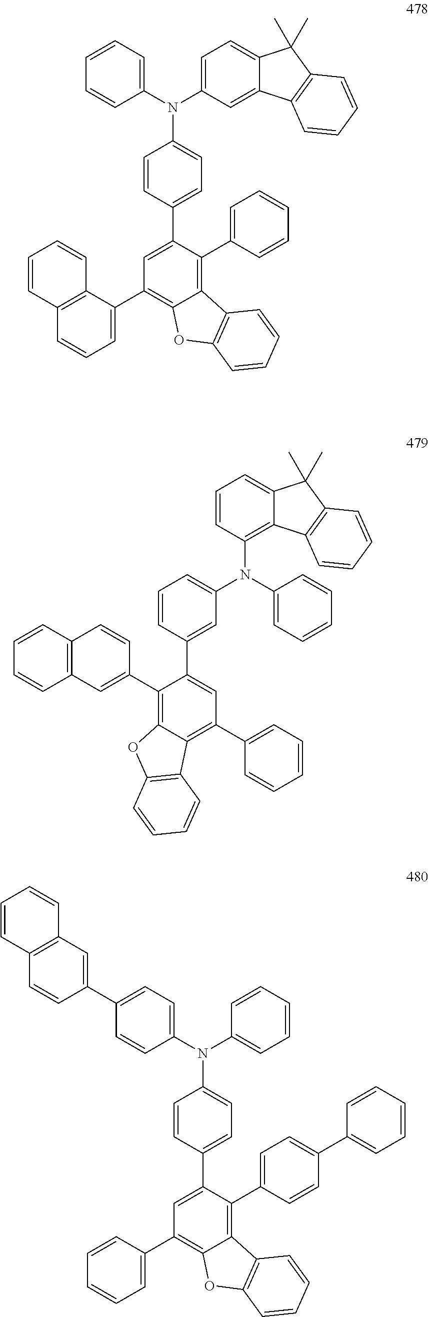 [SMILES: CC1(C)c2ccccc2-c2cc(N(c3ccccc3)c3ccc(-c4cc(-c5cccc6ccccc56)c5oc6ccccc6c5c4-c4ccccc4)cc3)ccc21 ; CC1(C)c2ccccc2-c2c(N(c3ccccc3)c3cccc(-c4cc(-c5ccccc5)c5c(oc6ccccc65)c4-c4ccc5ccccc5c4)c3)cccc21 ; c1ccc(-c2ccc(-c3c(-c4ccc(N(c5ccccc5)c5ccc(-c6ccc7ccccc7c6)cc5)cc4)cc(-c4ccccc4)c4oc5ccccc5c34)cc2)cc1]

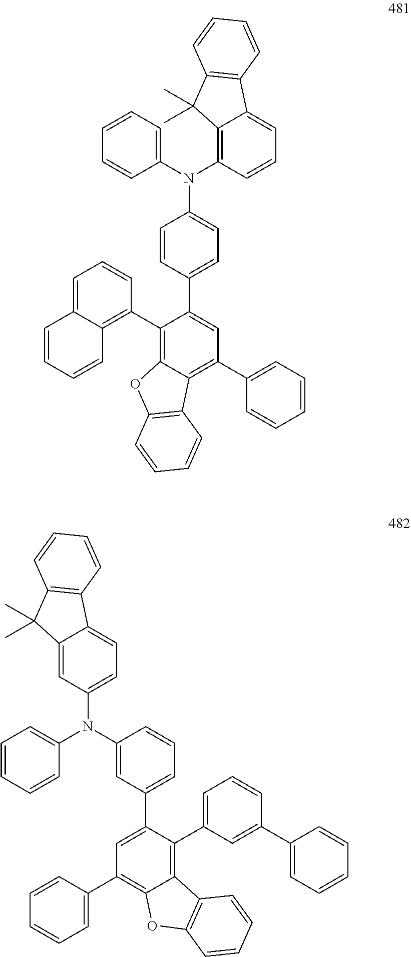 [SMILES: CC1(C)c2ccccc2-c2cccc(N(c3ccccc3)c3ccc(-c4cc(-c5ccccc5)c5c(oc6ccccc65)c4-c4cccc5ccccc45)cc3)c21 ; CC1(C)c2ccccc2-c2ccc(N(c3ccccc3)c3cccc(-c4cc(-c5ccccc5)c5oc6ccccc6c5c4-c4cccc(-c5ccccc5)c4)c3)cc21]

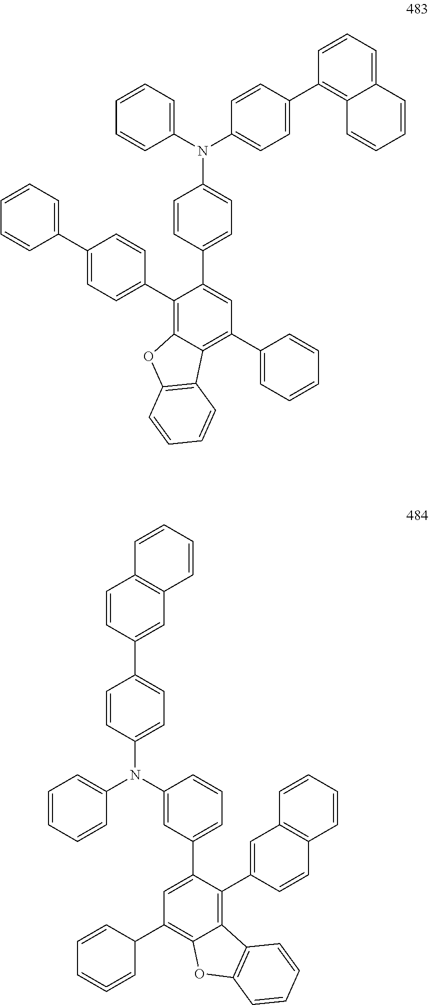 [SMILES: c1ccc(-c2ccc(-c3c(-c4ccc(N(c5ccccc5)c5ccc(-c6cccc7ccccc67)cc5)cc4)cc(-c4ccccc4)c4c3oc3ccccc34)cc2)cc1 ; C1=CC=CC(c2cc(-c3cccc(N(c4ccccc4)c4ccc(-c5ccc6ccccc6c5)cc4)c3)c(-c3ccc4ccccc4c3)c3c2oc2ccccc23)C=1]

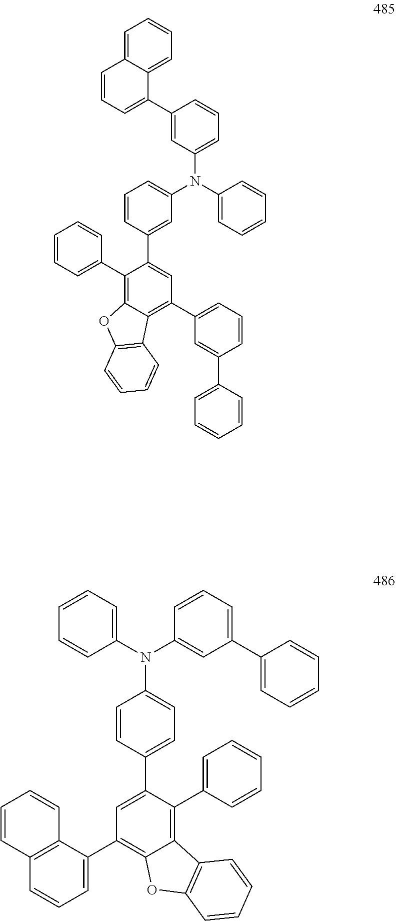 [SMILES: c1ccc(-c2cccc(-c3cc(-c4cccc(N(c5ccccc5)c5cccc(-c6cccc7ccccc67)c5)c4)c(-c4ccccc4)c4oc5ccccc5c34)c2)cc1 ; c1ccc(-c2cccc(N(c3ccccc3)c3ccc(-c4cc(-c5cccc6ccccc56)c5oc6ccccc6c5c4-c4ccccc4)cc3)c2)cc1]

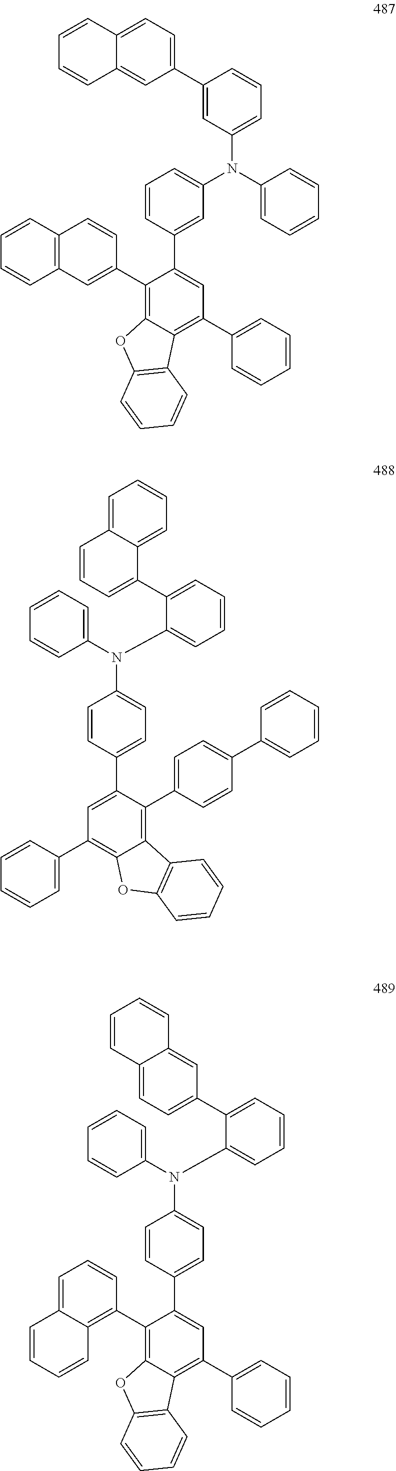 [SMILES: c1ccc(-c2cc(-c3cccc(N(c4ccccc4)c4cccc(-c5ccc6ccccc6c5)c4)c3)c(-c3ccc4ccccc4c3)c3oc4ccccc4c23)cc1 ; c1ccc(-c2ccc(-c3c(-c4ccc(N(c5ccccc5)c5ccccc5-c5cccc6ccccc56)cc4)cc(-c4ccccc4)c4oc5ccccc5c34)cc2)cc1 ; c1ccc(-c2cc(-c3ccc(N(c4ccccc4)c4ccccc4-c4ccc5ccccc5c4)cc3)c(-c3cccc4ccccc34)c3oc4ccccc4c23)cc1]

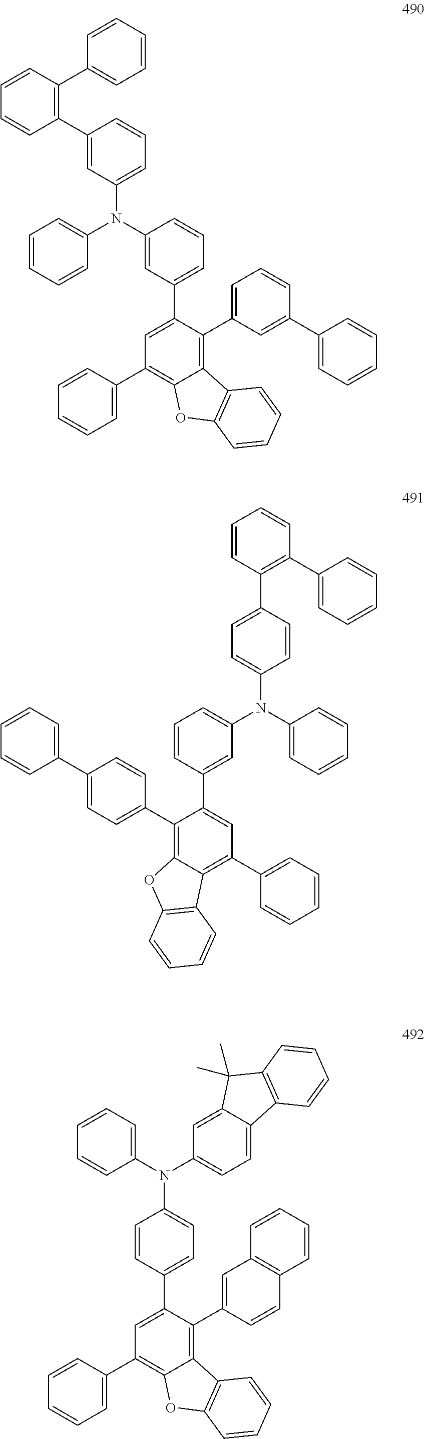 [SMILES: c1ccc(-c2cccc(-c3c(-c4cccc(N(c5ccccc5)c5cccc(-c6ccccc6-c6ccccc6)c5)c4)cc(-c4ccccc4)c4oc5ccccc5c34)c2)cc1 ; c1ccc(-c2ccc(-c3c(-c4cccc(N(c5ccccc5)c5ccc(-c6ccccc6-c6ccccc6)cc5)c4)cc(-c4ccccc4)c4c3oc3ccccc34)cc2)cc1 ; CC1(C)c2ccccc2-c2ccc(N(c3ccccc3)c3ccc(-c4cc(-c5ccccc5)c5oc6ccccc6c5c4-c4ccc5ccccc5c4)cc3)cc21]

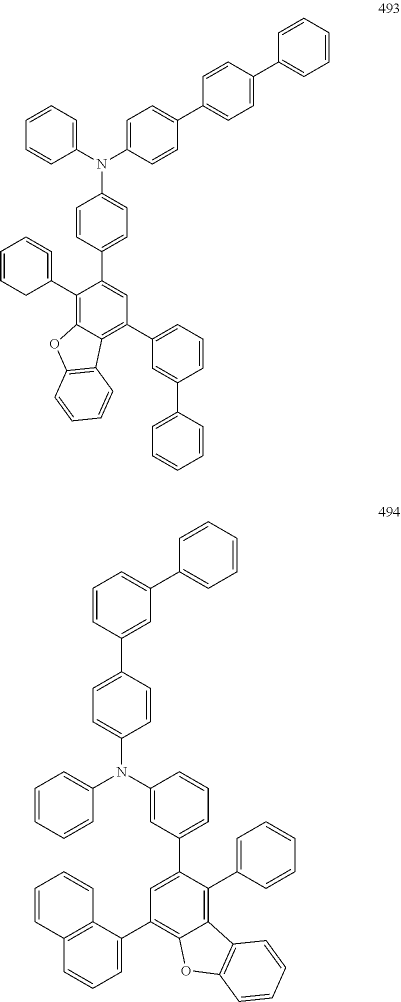 [SMILES: C1=CC=CCC=1c1c(-c2ccc(N(c3ccccc3)c3ccc(-c4ccc(-c5ccccc5)cc4)cc3)cc2)cc(-c2cccc(-c3ccccc3)c2)c2c1oc1ccccc12 ; c1ccc(-c2cccc(-c3ccc(N(c4ccccc4)c4cccc(-c5cc(-c6cccc7ccccc67)c6oc7ccccc7c6c5-c5ccccc5)c4)cc3)c2)cc1]

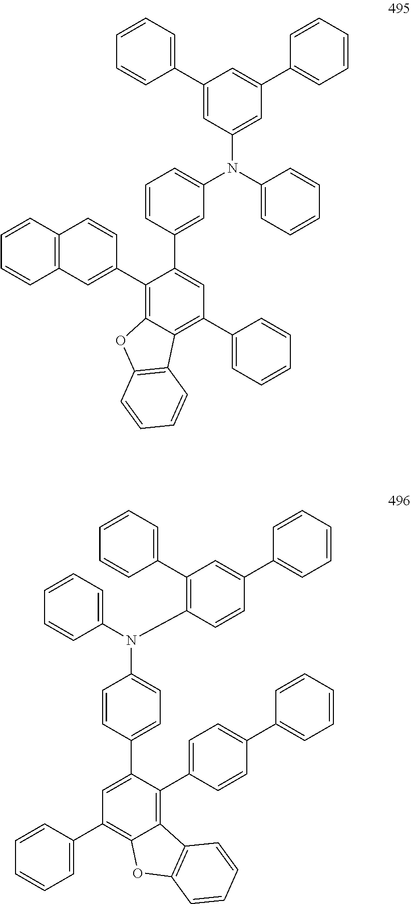 [SMILES: c1ccc(-c2cc(-c3ccccc3)cc(N(c3ccccc3)c3cccc(-c4cc(-c5ccccc5)c5c(oc6ccccc65)c4-c4ccc5ccccc5c4)c3)c2)cc1 ; c1ccc(-c2ccc(-c3c(-c4ccc(N(c5ccccc5)c5ccc(-c6ccccc6)cc5-c5ccccc5)cc4)cc(-c4ccccc4)c4oc5ccccc5c34)cc2)cc1]

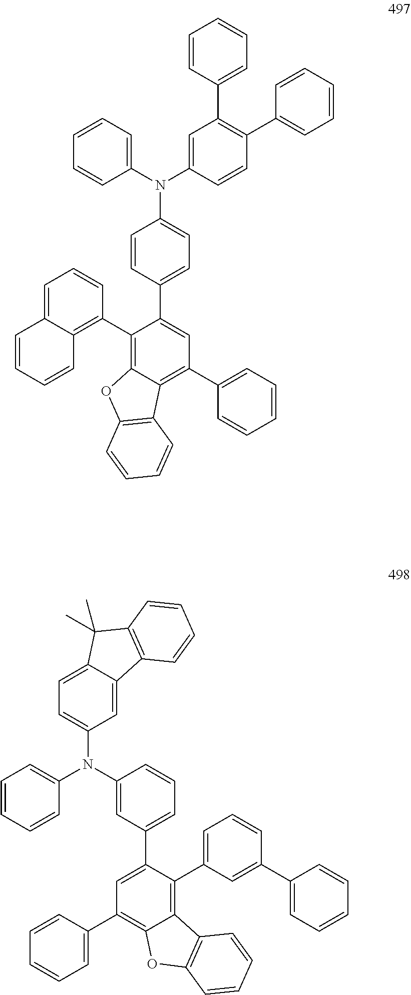 [SMILES: c1ccc(-c2ccc(N(c3ccccc3)c3ccc(-c4cc(-c5ccccc5)c5c(oc6ccccc65)c4-c4cccc5ccccc45)cc3)cc2-c2ccccc2)cc1 ; CC1(C)c2ccccc2-c2cc(N(c3ccccc3)c3cccc(-c4cc(-c5ccccc5)c5oc6ccccc6c5c4-c4cccc(-c5ccccc5)c4)c3)ccc21]

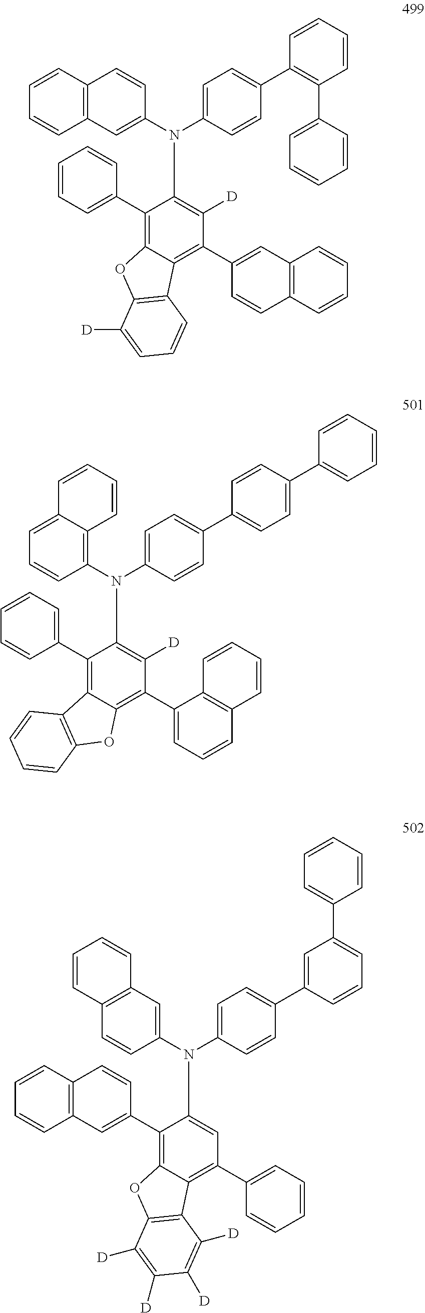 [SMILES: [2H]c1c(N(c2ccc(-c3ccccc3-c3ccccc3)cc2)c2ccc3ccccc3c2)c(-c2ccccc2)c2oc3c([2H])cccc3c2c1-c1ccc2ccccc2c1 ; [2H]c1c(N(c2ccc(-c3ccc(-c4ccccc4)cc3)cc2)c2cccc3ccccc23)c(-c2ccccc2)c2c(oc3ccccc32)c1-c1cccc2ccccc12 ; [2H]c1c([2H])c([2H])c2c(oc3c(-c4ccc5ccccc5c4)c(N(c4ccc(-c5cccc(-c6ccccc6)c5)cc4)c4ccc5ccccc5c4)cc(-c4ccccc4)c32)c1[2H]]

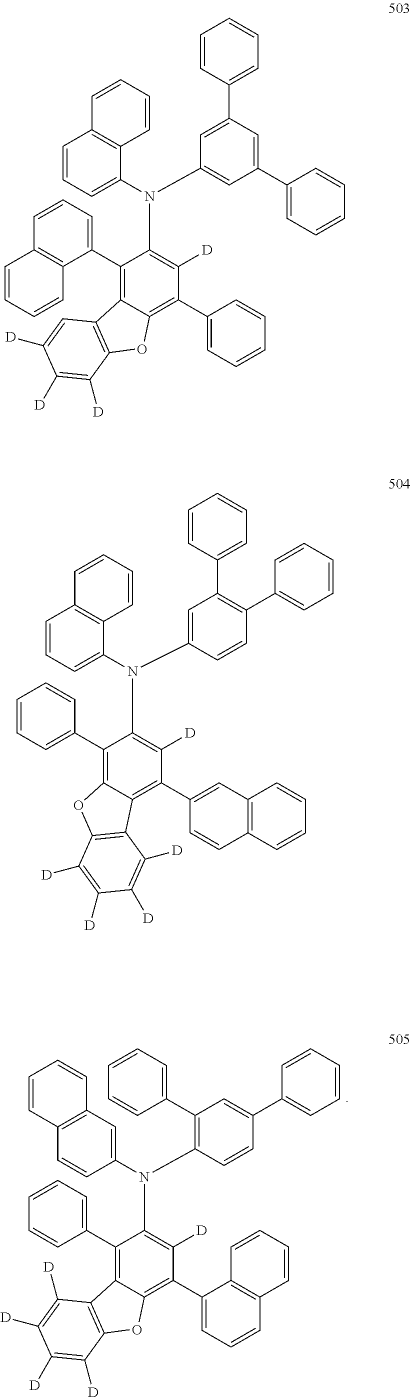 [SMILES: [2H]c1cc2c(oc3c(-c4ccccc4)c([2H])c(N(c4cc(-c5ccccc5)cc(-c5ccccc5)c4)c4cccc5ccccc45)c(-c4cccc5ccccc45)c32)c([2H])c1[2H] ; [2H]c1c([2H])c([2H])c2c(oc3c(-c4ccccc4)c(N(c4ccc(-c5ccccc5)c(-c5ccccc5)c4)c4cccc5ccccc45)c([2H])c(-c4ccc5ccccc5c4)c32)c1[2H] ; [2H]c1c([2H])c([2H])c2c(oc3c(-c4cccc5ccccc45)c([2H])c(N(c4ccc5ccccc5c4)c4ccc(-c5ccccc5)cc4-c4ccccc4)c(-c4ccccc4)c32)c1[2H]]

11. An electronic element, comprising an anode and a cathode disposed opposite to each other, and a functional layer disposed between the anode and the cathode; wherein the functional layer comprises the organic compound according to claim 1.

12. The electronic element according to claim 11, wherein the functional layer comprises an organic light-emitting layer and a hole transport layer, wherein the organic light-emitting layer comprises the organic compound and/or the hole transport layer comprises the organic compound;
optionally, the electronic element is an organic electroluminescent device or a photoelectric conversion device;
optionally, the electronic element is a red organic electroluminescent device.

13. An electronic apparatus, comprising the electronic element of claim 11.
